(19) United States
(12) Patent Application Publication
(10) Pub. No.: US 2023/0242484 A1
(43) Pub. Date: Aug. 3, 2023

(54) NITROGEN-CONTAINING COMPOUND, AND ELECTRONIC ELEMENT AND ELECTRONIC APPARATUS USING SAME

(71) Applicant: Shaanxi Lighte Optoelectronics Material Co., Ltd., Xi'an (CN)

(72) Inventors: Tiantian MA, Xi'an (CN); Min YANG, Xi'an (CN); Peng NAN, Xi'an (CN)

(21) Appl. No.: 18/011,804

(22) PCT Filed: Oct. 19, 2021
(86) PCT No.: PCT/CN2021/124790

§ 371 Date: Dec. 20, 2022

(30) Foreign Application Priority Data
Oct. 21, 2020 (CN) 202011135326.7

(51) Int. Cl. C07D 211/54; H10K 85/60; C07D 405/12; C07D 409/12; C07D 409/14; C07D 401/04; H10K 50/18

(52) U.S. Cl. CPC C07D 211/54; H10K 85/6572; H10K 85/636; C07D 405/12; H10K 85/6574; C07D 409/12; H10K 85/6576; H10K 85/615; H10K 85/622; C07D 409/14; H10K 85/654; C07D 401/04; H10K 50/181

(57) ABSTRACT

The present disclosure belongs to the technical field of organic materials, and specifically disclosed are a nitrogen-containing compound, and an electronic element and electronic apparatus using the same, the nitrogen-containing compound having a structure represented by a formula F-1. When the compound of the present disclosure is used as an electron blocking layer for manufacturing an organic electroluminescent device, the service life of the organic electroluminescent device may be effectively prolonged, and the luminous efficiency or driving voltage may be improved to a certain extent.

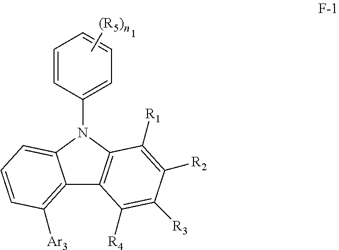

### CROSS REFERENCE TO RELATED APPLICATIONS

The present application claims the priority of Chinese Patent Application No. 202011135326.7, filed on Oct. 21, 2020, the contents of which are incorporated herein by reference in their entirety as part of the present application.

### FIELD

The present disclosure belongs to the technical field of organic materials, and specifically provides a nitrogen-containing compound, and an electronic element and electronic apparatus using the same.

### BACKGROUND

With the development of an electronic technology and the progress of material science, electronic components for realizing electroluminescence or photoelectric conversion are more and more widely used. Such electronic component typically includes a cathode and an anode which are oppositely disposed, and a functional layer disposed between the cathode and the anode. The functional layer consists of a plurality of organic or inorganic film layers, and generally includes an energy conversion layer, a hole transport layer between the energy conversion layer and the anode, and an electron transport layer between the energy conversion layer and the cathode.

Taking an organic electroluminescent device as an example, the organic electroluminescent device generally includes an anode, a hole transport layer, an electroluminescent layer as an energy conversion layer, an electron transport layer, and a cathode which are sequentially stacked. When a voltage is applied to the cathode and the anode, an electric field is generated between the two electrodes, electrons on a cathode side move towards the electroluminescent layer and holes on an anode side also move towards the light-emitting layer under the action of the electric field, the electrons and the holes are combined in the electroluminescent layer to form excitons, the excitons are in an excited state and release energy outwards, and then the electroluminescent layer emits light outwards.

At present, there are problems such as reduced luminous efficiency and shortened service life during use of the organic electroluminescent device, resulting in a decrease in performance of the organic electroluminescent device.

### SUMMARY

Aiming at the above problems existing in the prior art, the present disclosure aims to provide a nitrogen-containing compound, and an electronic element and electronic apparatus using the same. The nitrogen-containing compound can be used in an organic electroluminescent device to improve the performance of the organic electroluminescent device.

To achieve the above purpose, in a first aspect, the present disclosure provides a nitrogen-containing compound, having a structure represented by a formula F-1:

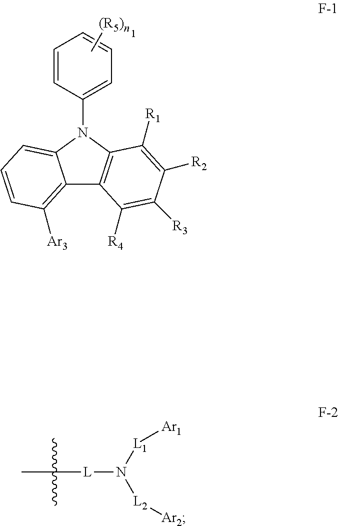

wherein [symbol] represents a chemical bond;

L, L₁ and L₂ are the same as or different from each other, and are respectively and independently selected from a single bond, substituted or unsubstituted arylene with 6 to 30 carbon atoms, and substituted or unsubstituted heteroarylene with 3 to 30 carbon atoms;

Ar₁ and Ar₂ are the same as or different from each other, and are respectively and independently selected from substituted or unsubstituted aryl with 6 to 40 carbon atoms, and substituted or unsubstituted heteroaryl with 3 to 30 carbon atoms;

Ar₃ is selected from substituted or unsubstituted aryl with 6 to 20 carbon atoms;

R₁, R₂, R₃, and R₄ are the same as or different from each other, and are respectively and independently selected from hydrogen or a group represented by a formula F-2, and one of R₁, R₂, R₃, and R₄ is the group represented by the formula F-2;

R₅ is selected from deuterium, cyano, a halogen group, substituted or unsubstituted alkyl with 1 to 10 carbon atoms, substituted or unsubstituted cycloalkyl with 3 to 20 carbon atoms, substituted or unsubstituted aryl with 6 to 30 carbon atoms, and substituted or unsubstituted heteroaryl with 3 to 30 carbon atoms;

n₁ represents the number of R₅, and n₁ is 0, 1, 2, 3, 4, or 5;

substituents in L, L₁, L₂, Ar₁, Ar₂, and R₅ are each independently selected from deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, alkenyl with 2 to 6 carbon atoms, alkynyl with 2 to 6 carbon atoms, cycloalkyl with 3 to 10 carbon atoms, heterocycloalkyl with 2 to 10 carbon atoms, cycloalkenyl with 5 to 10 carbon atoms, heterocycloalkenyl with 4 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms;

substituent in Ar₃ is selected from deuterium, a halogen group, cyano, and phenyl.

In a second aspect, the present disclosure provides an electronic element, comprising an anode and a cathode which is arranged oppositely to the anode, and a functional layer disposed between the anode and the cathode; and the functional layer comprises the nitrogen-containing compound of the first aspect of the present disclosure; and

preferably, the functional layer comprises an electron blocking layer, and the electron blocking layer comprises the nitrogen-containing compound.

In a third aspect, the present disclosure provides an electronic apparatus, comprising the electronic element of the second aspect of the present disclosure.

The nitrogen-containing compound of the present disclosure has a molecular structure with a carbazole derivative as a parent core bonded to an aromatic amine group. According to the compounds, the stability and hole transport properties of the whole molecule are improved by the synergistic action of the parent core with the surrounding hole transport groups. The arylamine structure in the nitrogen-containing compound can increase the transport efficiency of holes in a device and block electrons within a light-emitting layer, greatly increasing a carrier recombination rate; whereas the carbazole structure as the parent core has a large rigid plane, the group is relatively stable, and at the same time, the steric hindrance of the compounds is increased as a whole by introducing an aromatic substituent at the 4-position of carbazole, and the intermolecular stacking of the material can be further adjusted, effectively increasing the glass transition temperature of the compounds, and making it difficult to crystallize, thus improving the thermal stability of the material. When the nitrogen-containing compound of the present disclosure is used as an electron blocking layer for manufacturing an organic electroluminescent device, the service life of the organic electroluminescent device can be effectively prolonged, and the luminous efficiency or driving voltage can be improved to a certain extent.

### BRIEF DESCRIPTION OF THE DRAWINGS

FIG. 1 is a structural schematic diagram of an organic electroluminescent device according to one embodiment of the present disclosure.

FIG. 2 is a structural schematic diagram of a first electronic apparatus according to one embodiment of the present disclosure.

FIG. 3 is a structural schematic diagram of a photoelectric conversion device according to one embodiment of the present disclosure.

FIG. 4 is a structural schematic diagram of a second electronic apparatus according to one embodiment of the present disclosure.

### DESCRIPTION OF REFERENCE SIGNS

100, anode; 200, cathode; 300, functional layer; 310, hole injection layer; 321, hole transport layer; 322, electron blocking layer; 330, organic light-emitting layer; 340, electron transport layer; 350, electron injection layer; 360, photoelectric conversion layer; 400, first electronic apparatus; and 500, second electronic apparatus.

### DETAILED DESCRIPTION

The specific embodiments of the present disclosure will be described in detail below with reference to the accompanying drawings. It should be understood that the specific embodiments described here are only used to illustrate and explain the present disclosure, but are not intended to limit the present disclosure.

In a first aspect, the present disclosure provides a nitrogen-containing compound, having a structure represented by a formula F-1:

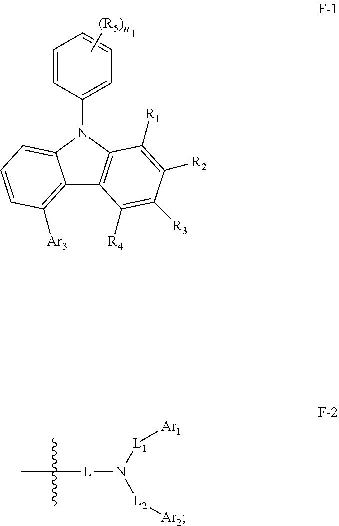

Wherein [symbol] represents a chemical bond;

L, L₁ and L₂ are the same as or different from each other, and are respectively and independently selected from a single bond, substituted or unsubstituted arylene with 6 to 30 carbon atoms, and substituted or unsubstituted heteroarylene with 3 to 30 carbon atoms;

Ar₁ and Ar₂ are the same as or different from each other, and are respectively and independently selected from substituted or unsubstituted aryl with 6 to 40 carbon atoms, and substituted or unsubstituted heteroaryl with 3 to 30 carbon atoms;

Ar₃ is selected from substituted or unsubstituted aryl with 6 to 20 carbon atoms;

R₁, R₂, R₃, and R₄ are the same as or different from each other, and are respectively and independently selected from hydrogen or a group represented by a formula F-2, and one of R₁, R₂, R₃, and R₄ is the group represented by the formula F-2;

R₅ is selected from deuterium, cyano, a halogen group, substituted or unsubstituted alkyl with 1 to 10 carbon atoms, substituted or unsubstituted cycloalkyl with 3 to 20 carbon atoms, substituted or unsubstituted aryl with 6 to 30 carbon atoms, and substituted or unsubstituted heteroaryl with 3 to 30 carbon atoms;

n₁ represents the number of R₅, and n₁ is 0, 1, 2, 3, 4, or 5;

substituents in L, L₁, L₂, Ar₁, Ar₂ and R₅ are each independently selected from deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, alkenyl with 2 to 6 carbon atoms, alkynyl with 2 to 6 carbon atoms, cycloalkyl with 3 to 10 carbon atoms, heterocycloalkyl with 2 to 10 carbon atoms, cycloalkenyl with 5 to 10 carbon atoms, heterocycloalkenyl with 4 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms;

substituent in Ar₃ is selected from deuterium, a halogen group, cyano, and phenyl.

In the present disclosure, “aryl with 6 to 20 carbon atoms which can be optionally substituted by 0, 1, 2, 3, 4, or 5 substituents selected from deuterium, fluorine, cyano, methyl, and tert-butyl” means that the aryl may be substituted by one or more of deuterium, fluorine, cyano, methyl, and tert-butyl, and may also not be substituted by deuterium, fluorine, cyano, methyl, or tert-butyl, and when the number of substituents in the aryl is greater than or equal to 2, the substituents may be the same or different.

Preferably, n₁ is 1.

In the present disclosure, the expressions “each . . . is independently”, “ . . . is respectively and independently” and “ . . . is independently selected from” can be interchanged, which should be understood in a broad sense, and may mean that specific options expressed by a same symbol in different groups do not influence each other, or may also mean that specific options expressed by a same symbol in a same group do not influence each other.

For example, the meaning of

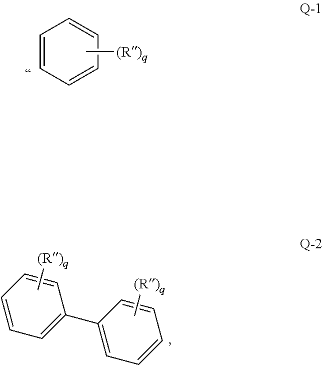

wherein each q is independently 0, 1, 2 or 3 and each R″ is independently selected from hydrogen, deuterium, fluorine, and chlorine“, means that: formula Q-1 represents that a benzene ring has q substituents R”, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the term “substituted or unsubstituted” means that a functional group described behind the term may or may not have substituents (the substituents below are collectively referred to as Rₓ for convenience of description). For example, “substituted or unsubstituted aryl” refers to aryl with a substituent Rₓ or unsubstituted aryl. The above substituent, i.e. Rₓ, may be, for example, deuterium, a halogen group, cyano, heteroaryl with 3 to 20 carbon atoms, aryl with 6 to 20 carbon atoms which can be optionally substituted by 0, 1, 2, 3, 4, or 5 substituents selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, alkenyl with 2 to 6 carbon atoms, alkynyl with 2 to 6 carbon atoms, cycloalkyl with 3 to 10 carbon atoms, heterocycloalkyl with 2 to 10 carbon atoms, cycloalkenyl with 5 to 10 carbon atoms, heterocycloalkenyl with 4 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, or phosphinyloxy with 6 to 18 carbon atoms; when two substituents Rₓ are connected to a same atom, the two substituents Rₓ may independently be present or connected to each other to form a ring with the atom; when two adjacent substituents Rₓ are present on a functional group, the two adjacent substituents Rₓ may independently be present or fused to form a ring with the functional group to which they are connected.

In the present disclosure, the number of carbon atoms of a substituted or unsubstituted functional group refers to the number of all carbon atoms. For example, if L is selected from substituted arylene with 12 carbon atoms, the number of all carbon atoms of the arylene and substituents on the arylene is 12. For example: if Ar₁ is

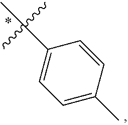 [SMILES: *c1ccc(C)cc1]

then the number of carbon atoms is 7; and if L is

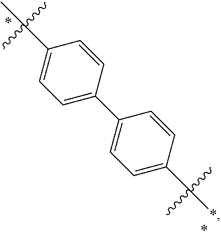

the number of carbon atoms is 12.

In the present disclosure, aryl refers to an optional functional group or substituent derived from an aromatic carbon ring. The aryl can be monocyclic aryl (e.g., phenyl) or polycyclic aryl, in other words, the aryl can be monocyclic aryl, fused aryl, two or more monocyclic aryl conjugatedly connected by carbon-carbon bond, monocyclic aryl and fused aryl conjugatedly connected by a carbon-carbon bond, or two or more fused aryl conjugatedly connected by carbon-carbon bonds. That is, unless specified otherwise, two or more aromatic groups conjugatedly connected by carbon-carbon bonds can also be regarded as aryl of the present disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se, and Si. For example, in the present disclosure, biphenyl, terphenyl, and the like are aryl. Examples of the aryl can include, but are not limited to, phenyl, naphthyl, fluorenyl, anthryl, phenanthryl, biphenyl, terphenyl, quaterphenyl, quinquephenyl, benzo[9,10]phenanthryl, pyrenyl, benzofluoranthenyl, chrysenyl, etc. “Aryl” in the present disclosure may contain 6 to 40 carbon atoms, in some embodiments, the number of carbon atoms in the aryl may be 6 to 25, in some other embodiments, the number of carbon atoms in the aryl may be 6 to 18, and in some other embodiments, the number of carbon atoms in the aryl may be 6 to 13. For example, the number of carbon atoms in the aryl can be 6, 12, 13, 14, 15, 18, 20, 24, 25, 30, 31, 32, 33, 34, 35, 36, or 40, and of course, the number of carbon atoms can also be other numbers, which will not be listed here. In the present disclosure, biphenyl can be understood as phenyl-substituted aryl and can also be understood as unsubstituted aryl.

In the present disclosure, the related arylene refers to a divalent group formed by further loss of one hydrogen atom of the aryl.

In the present disclosure, substituted aryl can be that one or two or more hydrogen atoms in the aryl are substituted by other groups such as a deuterium atom, a halogen group, cyano, aryl, heteroaryl, trialkylsilyl, alkyl, cycloalkyl, alkoxy, alkylthio, etc. Specific examples of heteroaryl-substituted aryl include, but are not limited to, dibenzofuranyl-substituted phenyl, dibenzothiophene-substituted phenyl, pyridine-substituted phenyl, and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and its substituents is 18.

In the present disclosure, specific examples of aryl as a substituent include, but are not limited to, phenyl, naphthyl, anthryl, phenanthryl, dimethylfluorenyl, biphenyl, diphenylfluorenyl, spirobifluorenyl, and the like.

In the present disclosure, fluorenyl may be substituted and two substituents may be bonded to each other to form a spiro structure, and specific embodiments include, but are not limited to, the following structures:

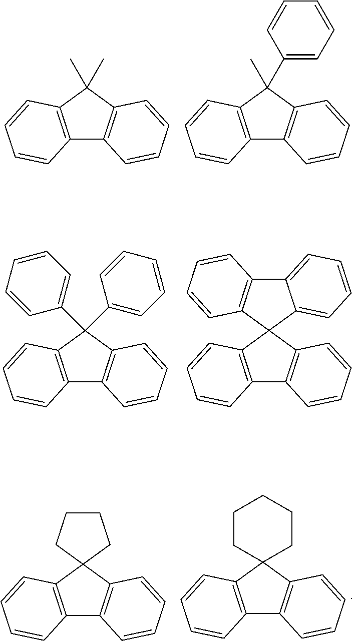

In the present disclosure, heteroaryl refers to a monovalent aromatic ring containing at least one heteroatom in the ring or its derivative, and the heteroatom can be at least one of B, O, N, P, Si, Se, and S. The heteroaryl may be monocyclic heteroaryl or polycyclic heteroaryl, in other words, the heteroaryl may be a single aromatic ring system or a plurality of aromatic ring systems conjugatedly connected by carbon-carbon bond, and where any one aromatic ring system is an aromatic monocyclic ring or an aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, and N-phenylcarbazolyl, N-pyridylcarbazolyl, N-methylcarbazolyl and the like, but is not limited to this. Among them, thienyl, furyl, phenanthrolinyl, etc. is heteroaryl of single aromatic ring system, and N-arylcarbazolyl, and N-heteroarylcarbazolyl is heteroaryl of the plurality of aromatic ring systems conjugatedly connected by carbon-carbon bond. “Heteroaryl” in the present disclosure may contain 3 to 30 carbon atoms, in some embodiments, the number of carbon atoms in the heteroaryl may be 3 to 30, in some other embodiments, the number of carbon atoms in the heteroaryl may be 3 to 20, and in some other examples, the number of carbon atoms in the heteroaryl may be 12 to 20. For example, the number of carbon atoms may be 3, 4, 5, 7, 12, 13, 18, 20, 24, 25 or 30, and of course, the number of carbon atoms may also be other numbers, which will not be listed here.

In the present disclosure, the related heteroarylene refers to a divalent group formed by further loss of one hydrogen atom of the heteroaryl.

In the present disclosure, substituted heteroaryl may be that one or two or more hydrogen atoms in the heteroaryl are substituted by other groups, such as a deuterium atom, a halogen group, cyano, aryl, heteroaryl, trialkylsilyl, alkyl, cycloalkyl, alkoxy, alkylthio, and the like. Specific examples of aryl-substituted heteroaryl include, but are not limited to, phenyl-substituted dibenzofuranyl, phenyl-substituted dibenzothienyl, N-phenylcarbazolyl, and the like. It should be understood that the number of carbon atoms of the substituted heteroaryl refers to the total number of carbon atoms of the heteroaryl and substituents on the heteroaryl.

In the present disclosure, specific examples of heteroaryl as a substituent include, but are not limited to, dibenzofuranyl, dibenzothienyl, carbazolyl, N-phenylcarbazolyl, phenanthrolinyl, and the like.

In the present disclosure, in the condition that “any two adjacent Rⱼ form a ring”, “any adjacent” can include the condition that there are two Rⱼ on a same atom and can also include the condition that two adjacent atoms each have one Rⱼ; when there are two Rj on the same atom, the two Rⱼ may form a saturated or unsaturated ring with the atom to which they are commonly connected; and when two adjacent atoms each have one Rⱼ, the two Rⱼ may be fused to form a ring. Similarly, any two adjacent substituents form a ring, which also has the same interpretation, which will not be repeated in the present disclosure.

In the present disclosure, an unpositioned connecting bond refers to a single bond “ [symbol] ” extending from a ring system, which indicates that one end of the connecting bond can be connected to any position in the ring system through which the bond penetrates, and the other end of the connecting bond is connected to the remaining part of a compound molecule.

For example, as shown in the formula (X) below, naphthyl represented by the formula (X) is connected to other positions of a molecule via two unpositioned connecting bonds penetrating a bicyclic ring, and its meaning includes any one possible connection mode represented by formulae (X-1) to (X-10).

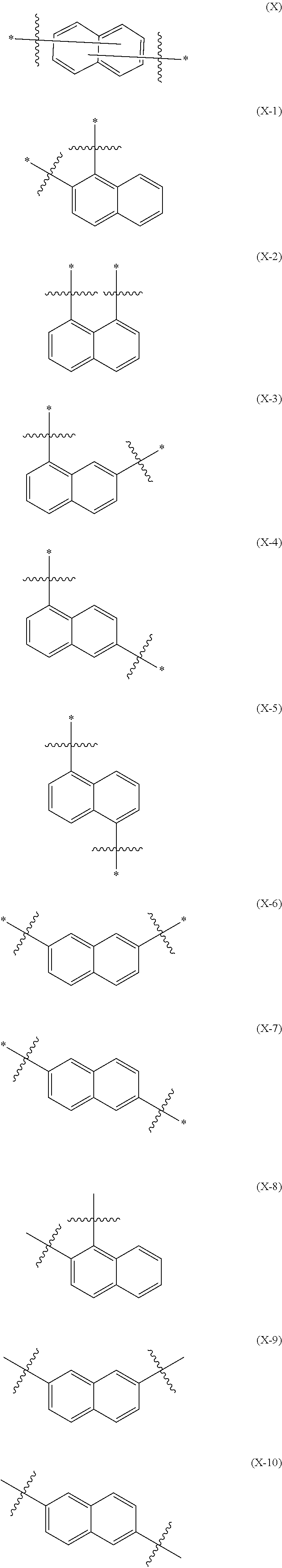

For another example, as shown in the formula (X′) below, phenanthryl represented by the formula (X′) is connected to other positions of a molecule via one unpositioned connecting bond extending from the middle of a benzene ring on one side, and its meaning includes any one possible connection mode represented by formulae (X′-1) to (X′-4).

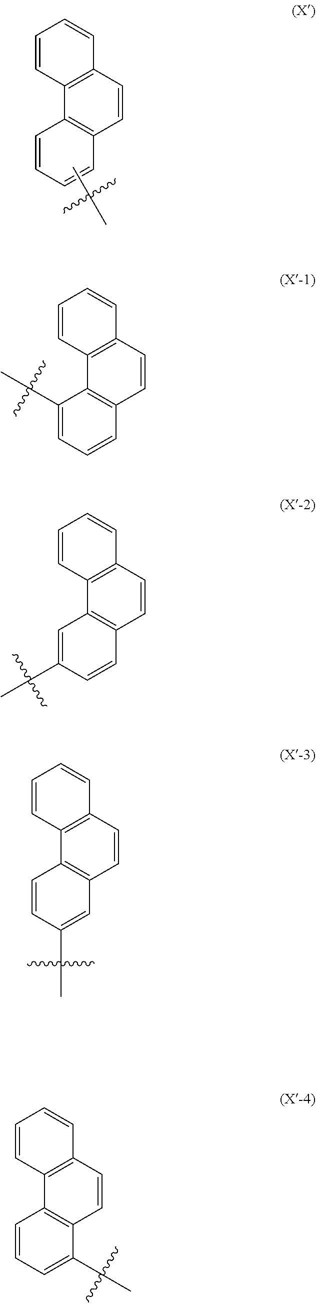

An unpositioned substituent in the present disclosure refers to a substituent connected through a single bond extending from the center of a ring system, which means that the substituent can be connected to any possible position in the ring system. For example, as shown in the formula (Y) below, the substituent R represented by the formula (Y) is connected to a quinoline ring via one unpositioned connecting bond, and its meaning includes any one possible connection mode represented by formulae (Y-1) to (Y-7).

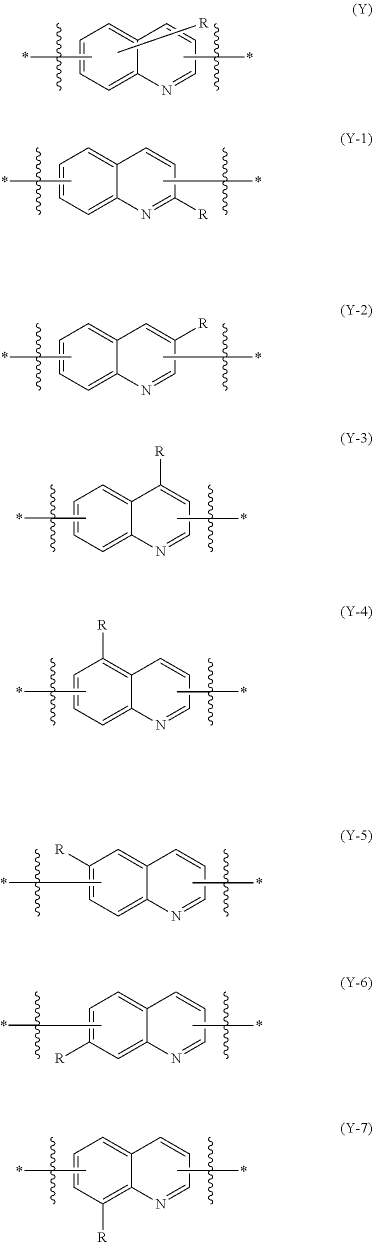

In the present disclosure, alkyl with 1 to 10 carbon atoms may include linear alkyl with 1 to 10 carbon atoms and branched alkyl with 3 to 10 carbon atoms, and the number of carbon atoms may be, for example, 1, 2, 3, 4, 5, 6, 7, 8, 9, or 10. Specific examples of the alkyl with 1 to 10 carbon atoms include, but are not limited to, methyl, ethyl, n-propyl, isopropyl, n-butyl, isobutyl, tert-butyl, n-pentyl, isopentyl, neopentyl, cyclopentyl, n-hexyl, heptyl, n-octyl, 2-ethylhexyl, nonyl, decyl, 3,7-dimethyloctyl, and the like.

In the present disclosure, the halogen group may include fluorine, iodine, bromine, chlorine, and the like.

In the present disclosure, specific examples of trialkylsilyl with 3 to 12 carbon atoms include, but are not limited to, trimethylsilyl, triethylsilyl, and the like.

In the present disclosure, specific examples of triarylsilyl with 18 to 24 carbon atoms include, but are not limited to, triphenylsilyl and the like.

In the present disclosure, specific examples of cycloalkyl with 3 to 20 carbon atoms include, but are not limited to, cyclopentyl, cyclohexyl, adamantyl, and the like.

Optionally, the nitrogen-containing compound is selected from compounds represented by any one of the following chemical formulae:

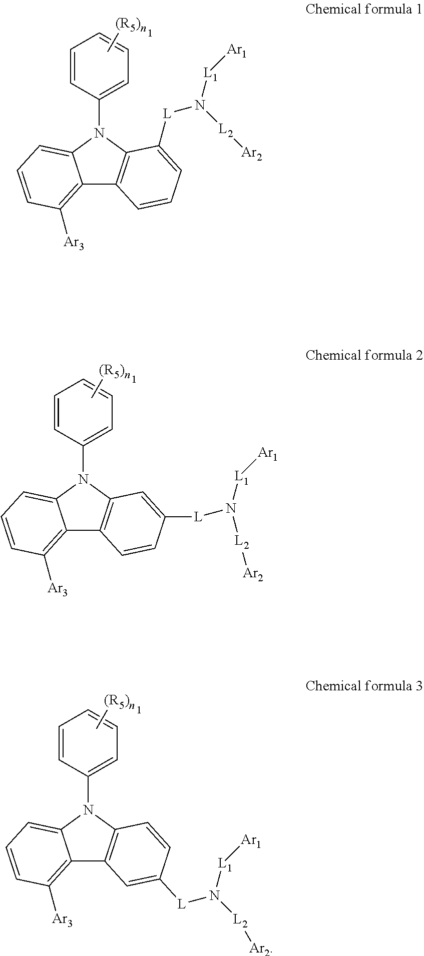

In one embodiment of the present disclosure, the L, L₁ and L₂ are the same as or different from each other, and are respectively and independently selected from a single bond, substituted or unsubstituted arylene with 6 to 25 carbon atoms, and substituted or unsubstituted heteroarylene with 3 to 25 carbon atoms.

Optionally, the L, L₁ and L₂ are the same as or different from each other, and are respectively and independently selected from a single bond, substituted or unsubstituted arylene with 6 to 20 carbon atoms, and substituted or unsubstituted heteroarylene with 12 to 20 carbon atoms.

Optionally, substituents in the L, L₁ and L₂ are the same or different, and are respectively and independently selected from deuterium, a halogen group, cyano, alkyl with 1 to 5 carbon atoms, aryl with 6 to 18 carbon atoms, and heteroaryl with 12 to 18 carbon atoms.

Specifically, substituents in the L, L₁ and L₂ are the same or different, and are respectively and independently selected from deuterium, fluorine, cyano, methyl, ethyl, n-propyl, isopropyl, tert-butyl, phenyl, naphthyl, biphenyl, terphenyl, dibenzofuranyl, dibenzothienyl, carbazolyl, N-phenylcarbazolyl, and the like.

In another embodiment of the present disclosure, the L, L₁ and L₂ are the same as or different from each other, and are respectively and independently selected from a single bond, substituted or unsubstituted phenylene, substituted or unsubstituted naphthylene, substituted or unsubstituted biphenylene, substituted or unsubstituted dimethylfluorenylene, substituted or unsubstituted dibenzofurylene, substituted or unsubstituted carbazolylene, substituted or unsubstituted dibenzothienylene, and substituted or unsubstituted N-phenylcarbazolylene.

Preferably, substituents in the L, L₁ and L₂ are the same or different, and are respectively and independently selected from deuterium, fluorine, cyano, methyl, ethyl, n-propyl, isopropyl, tert-butyl, phenyl, naphthyl, biphenyl, terphenyl, phenanthryl, dimethylfluorenyl, dibenzofuranyl, dibenzothienyl, carbazolyl, and N-phenylcarbazolyl.

In one embodiment of the present disclosure, the Ar₃ is selected from substituted or unsubstituted aryl with 6 to 12 carbon atoms, and preferably, substituent in Ar₃ is phenyl.

Further preferably, the Ar₃ is selected from unsubstituted phenyl, unsubstituted naphthyl, and unsubstituted biphenyl.

In one embodiment of the present disclosure, Ar₃ is selected from a group consisting of the following groups:

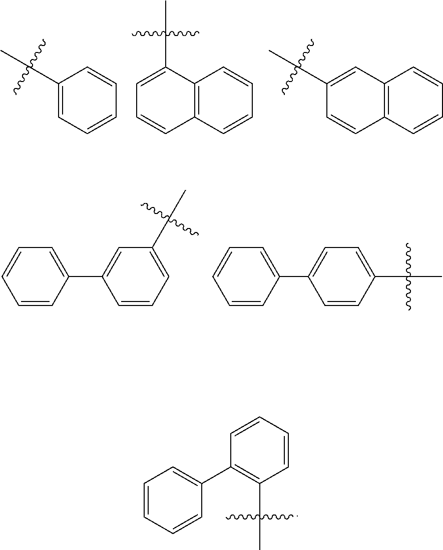

In one embodiment of the present disclosure, the L, L₁ and L₂ are each independently selected from a single bond or a substituted or unsubstituted group W, and the unsubstituted group W is selected from a group consisting of the following groups:

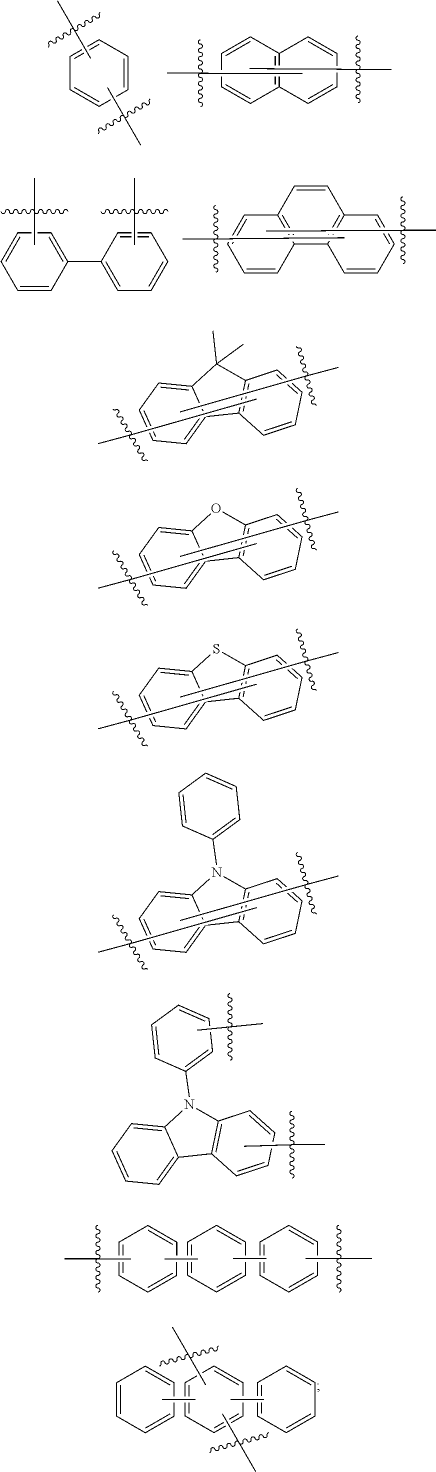

wherein [symbol] represents a chemical bond; the substituted group W has one or more substituents, and the substituents are each independently selected from deuterium, cyano, a halogen group, methyl, ethyl, n-propyl, isopropyl, tert-butyl, phenyl, naphthyl, biphenyl, phenanthryl, and anthryl; when the number of the substituents in the group W is greater than 1, the substituents are the same or different.

Optionally, the L, L₁ and L₂ are each independently selected from a single bond or a group consisting of the following groups:

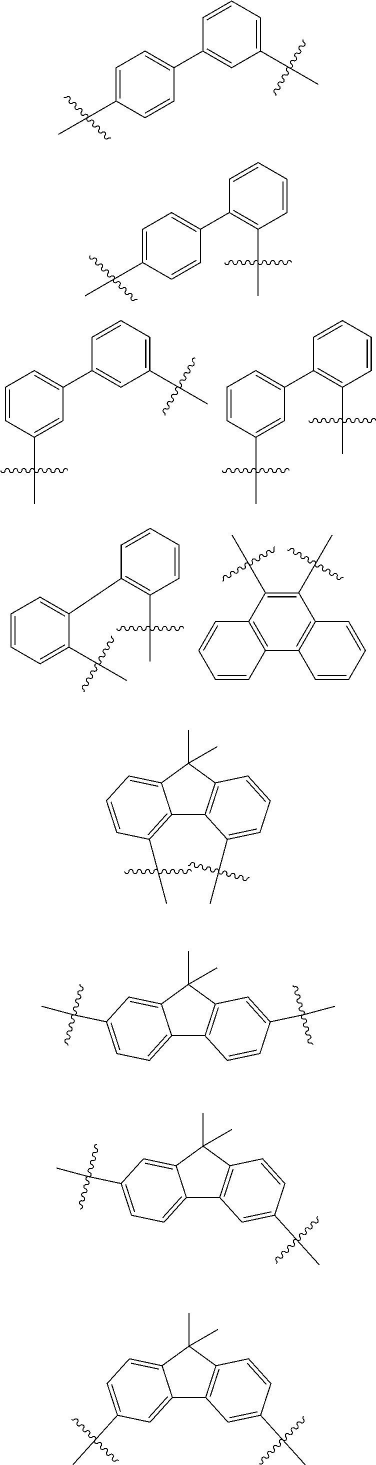

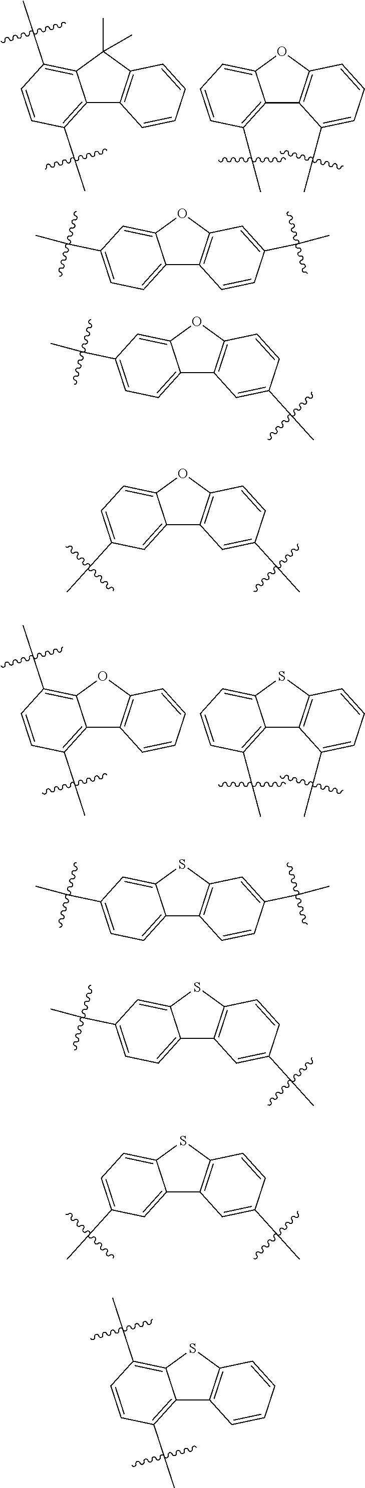

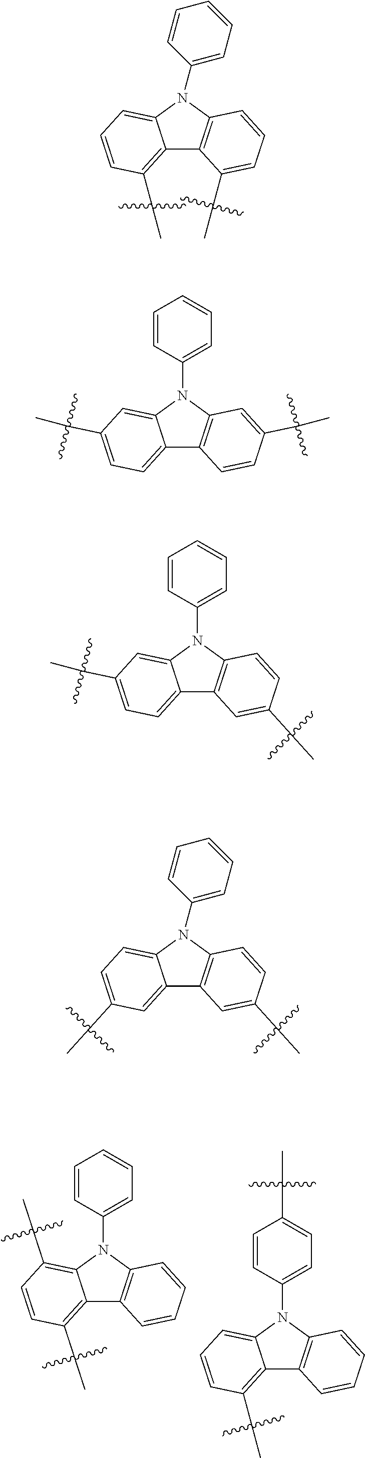

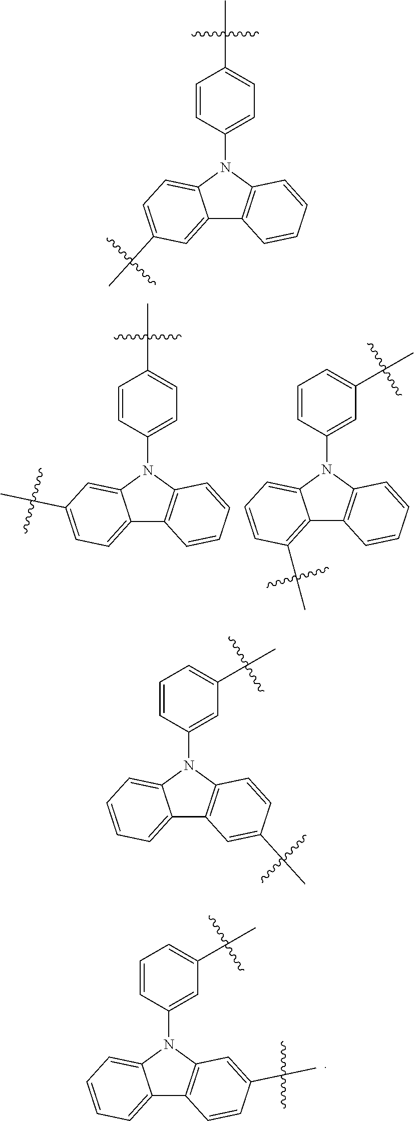

In one embodiment of the present disclosure, Ar₁ and Ar₂ are the same as or different from each other, and are respectively and independently selected from substituted or unsubstituted aryl with 6 to 36 carbon atoms and substituted or unsubstituted heteroaryl with 3 to 25 carbon atoms.

Optionally, Ar₁ and Ar₂ are the same as or different from each other, and are respectively and independently selected from substituted or unsubstituted aryl with 6 to 33 carbon atoms and substituted or unsubstituted heteroaryl with 3 to 25 carbon atoms.

Preferably, Ar₁ and Ar₂ are the same as or different from each other, and are respectively and independently selected from substituted or unsubstituted aryl with 6 to 33 carbon atoms, and substituted or unsubstituted heteroaryl with 3 to 20 carbon atoms.

Optionally, substituents in the Ar₁ and the Ar₂ are the same as or different from each other, and are respectively and independently selected from deuterium, a halogen group, cyano, alkyl with 1 to 5 carbon atoms, aryl with 6 to 20 carbon atoms, and heteroaryl with 12 to 20 carbon atoms.

Further optionally, substituents in the Ar₁ and the Ar₂ are the same as or different from each other, and are respectively and independently selected from deuterium, a halogen group, cyano, alkyl with 1 to 5 carbon atoms, aryl with 6 to 18 carbon atoms, and heteroaryl with 12 to 18 carbon atoms.

Specifically, substituents in the Ar₁ and Ar₂ include, but are not limited to, deuterium, fluorine, cyano, methyl, ethyl, n-propyl, isopropyl, tert-butyl, phenyl, naphthyl, biphenyl, terphenyl, dibenzofuranyl, dibenzothienyl, carbazolyl, N-phenylcarbazolyl, and the like.

In one embodiment of the present disclosure, the Ar₁ and Ar₂ are each independently selected from a substituted or unsubstituted group V, and the unsubstituted group V is selected from a group consisting of the following groups:

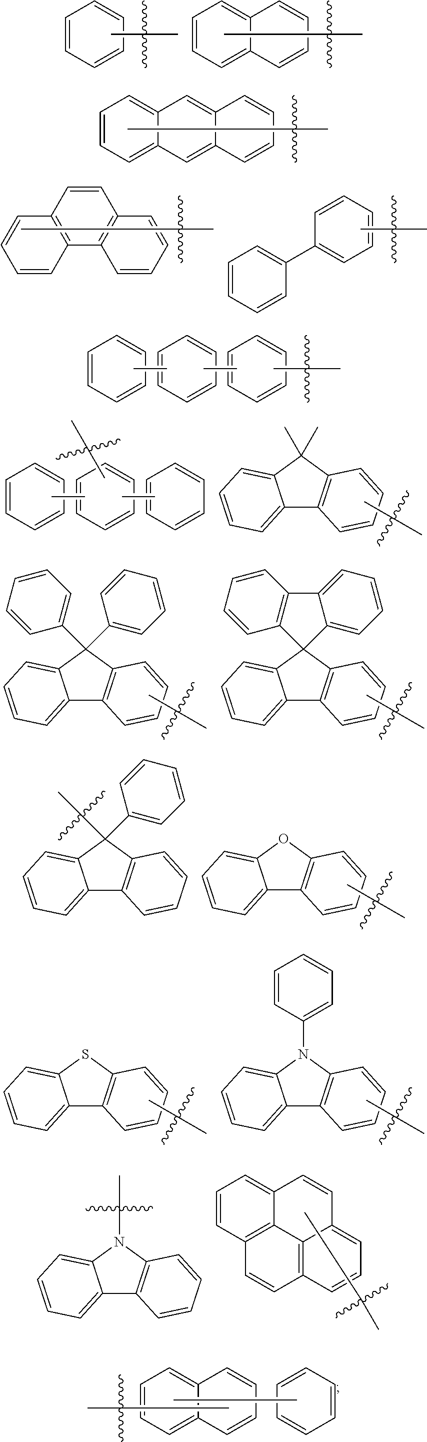

where [symbol] represents a chemical bond; the substituted group V has one or more substituents, and the substituents are each independently selected from deuterium, cyano, a halogen group, methyl, ethyl, n-propyl, isopropyl, tert-butyl, phenyl, naphthyl, biphenyl, phenanthryl, anthryl, dibenzofuranyl, dibenzothienyl, carbazolyl, and N-phenylcarbazolyl; when the number of the substituents in the group V is greater than 1, the substituents are the same or different.

Optionally, the Ar₁ and Ar₂ are respectively and independently selected from a group consisting of the following groups:

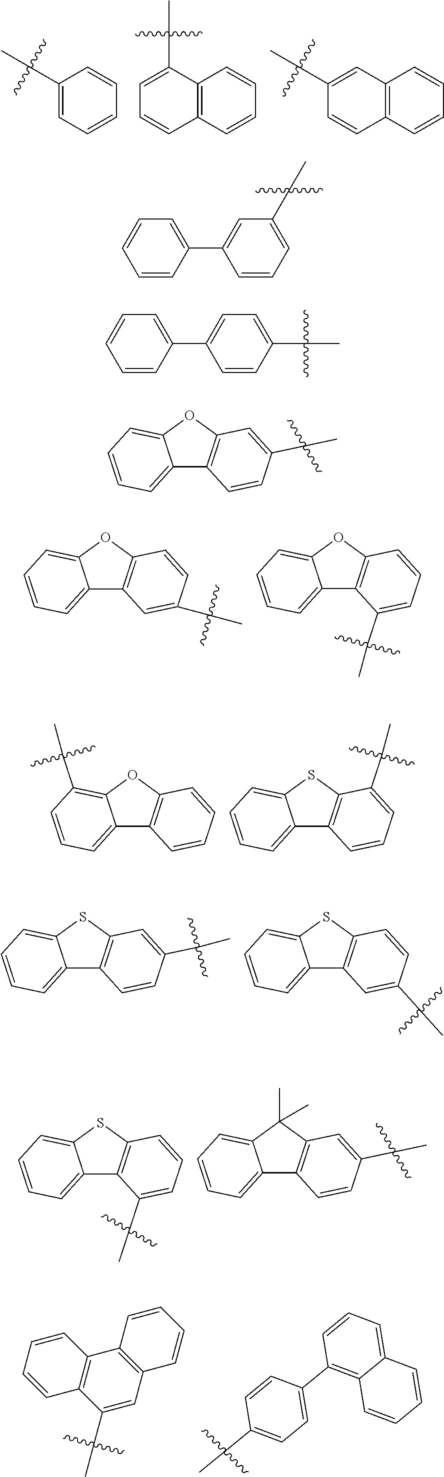

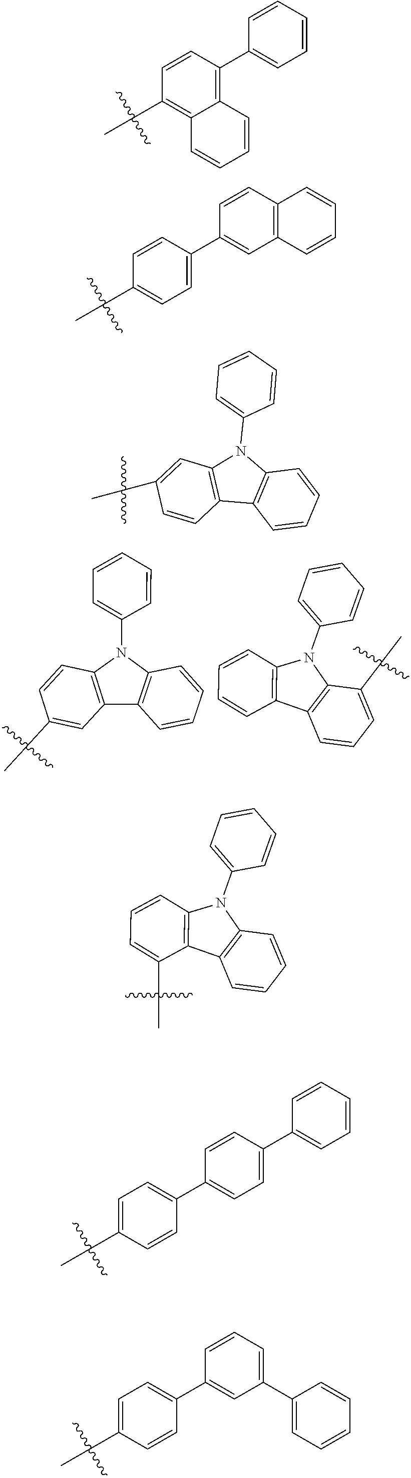

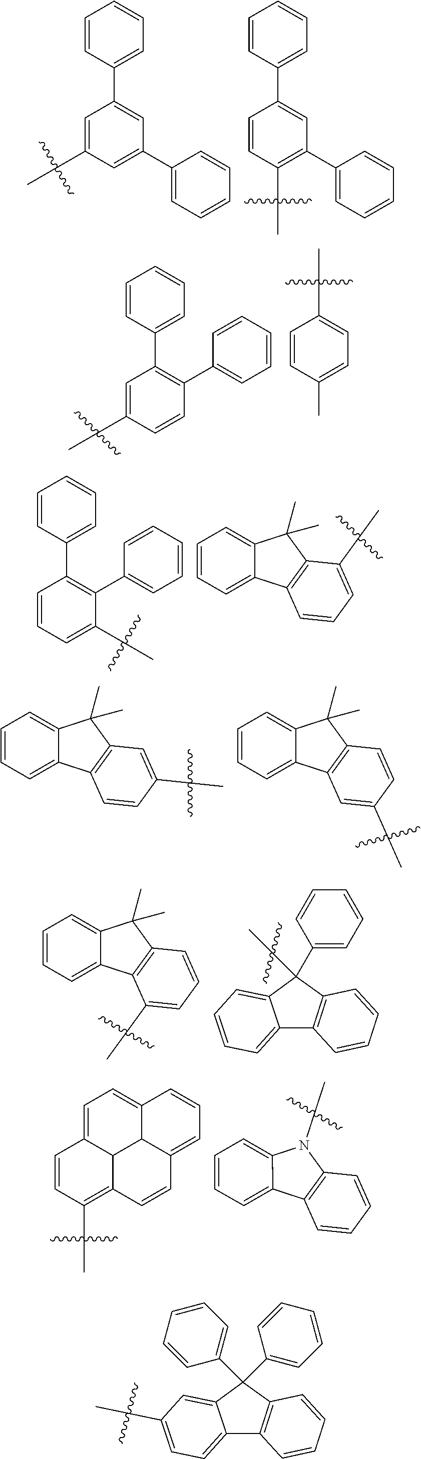

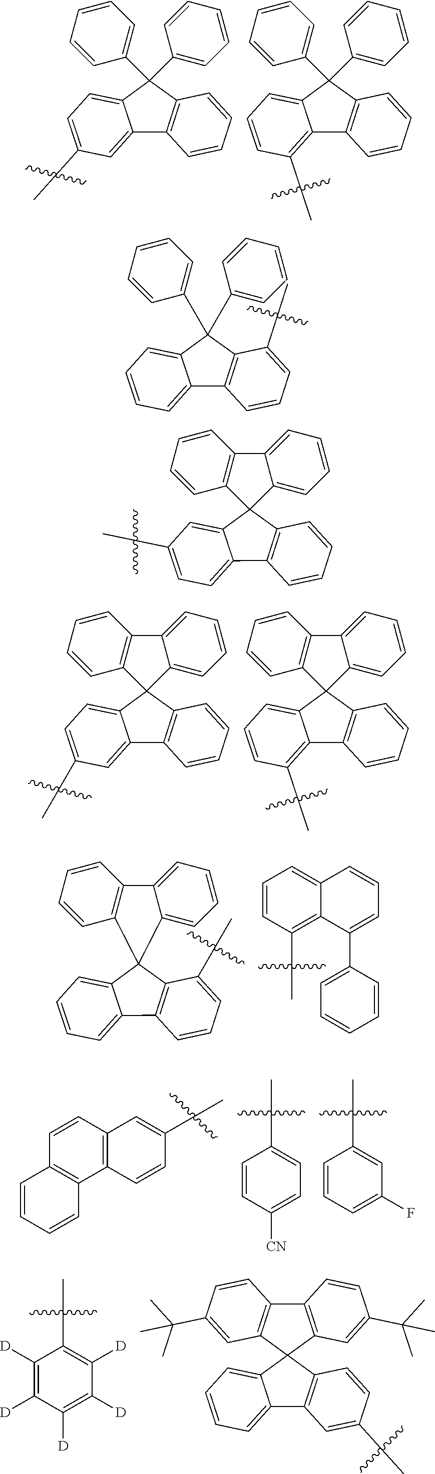

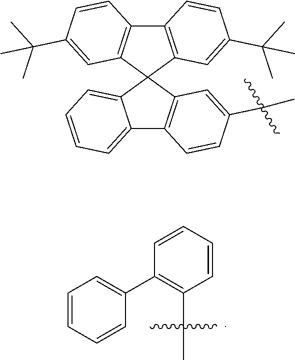 [SMILES: *c1ccc2c(c1)C1(c3ccccc3-2)c2cc(C(C)(C)C)ccc2-c2ccc(C(C)(C)C)cc21 ; *c1ccccc1-c1ccccc1]

In the present disclosure, optionally, the nitrogen-containing compound is selected from a group consisting of the following compounds:

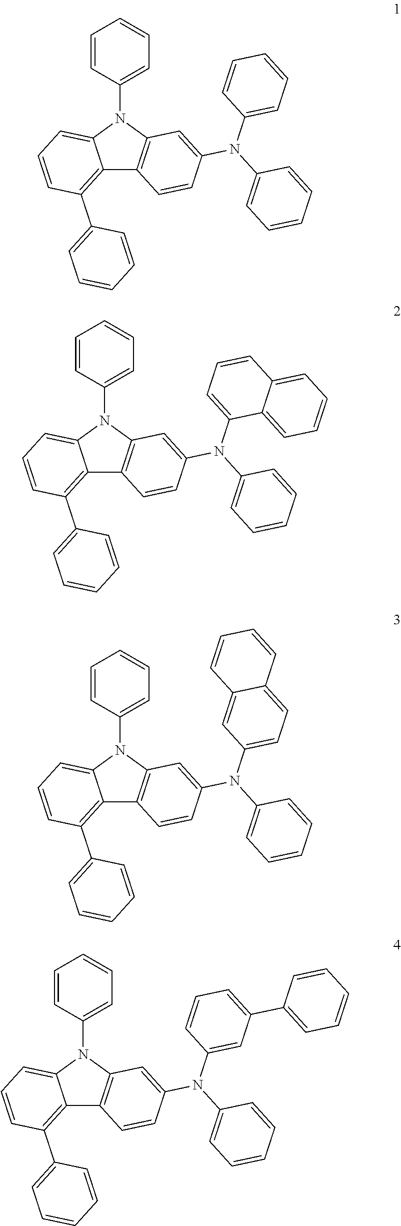

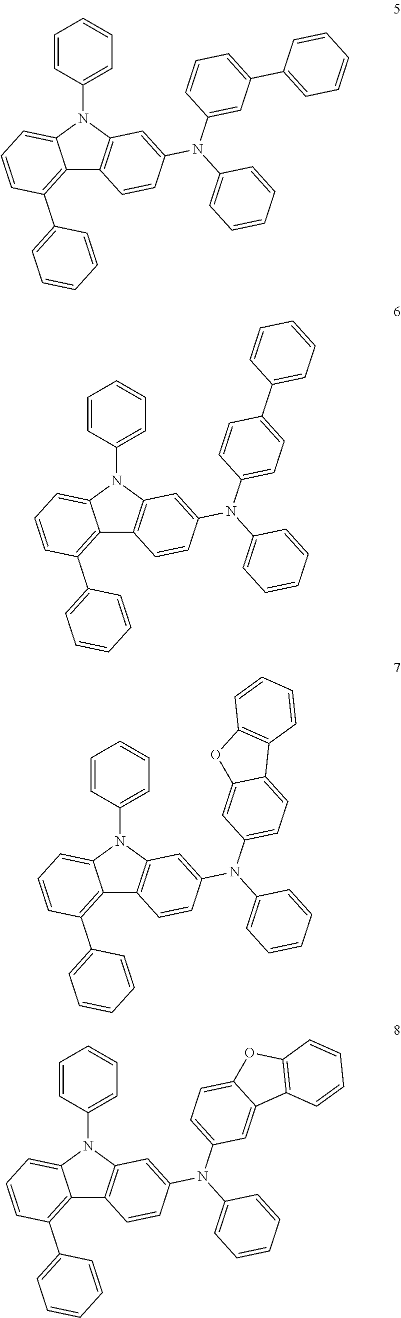

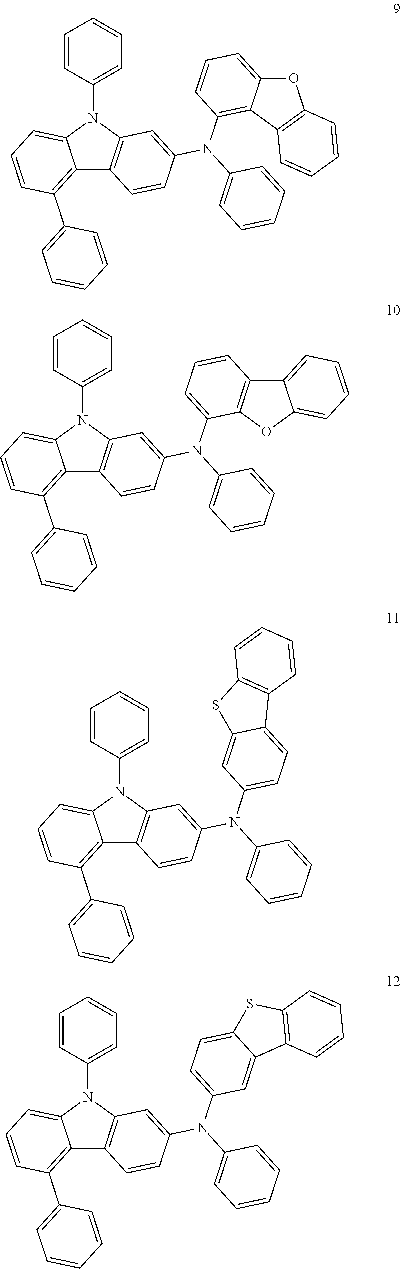

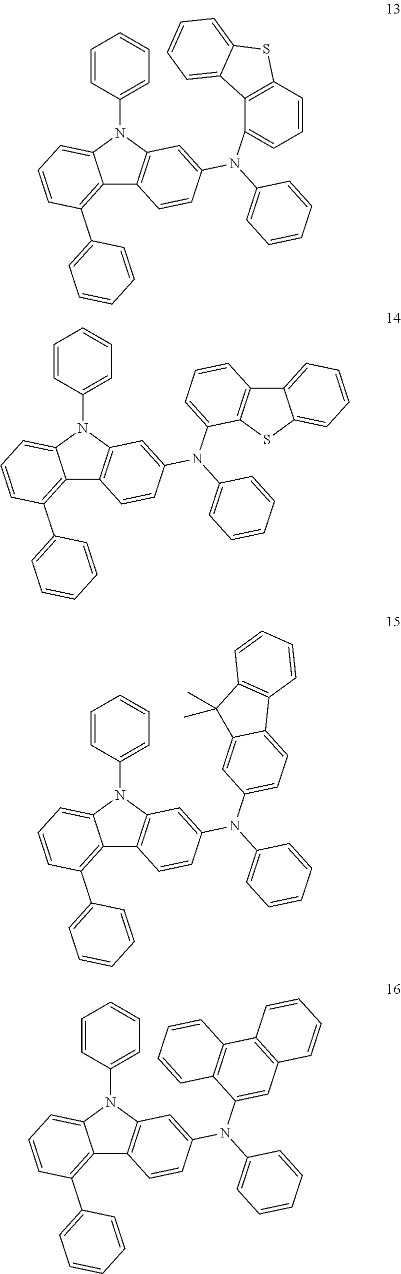

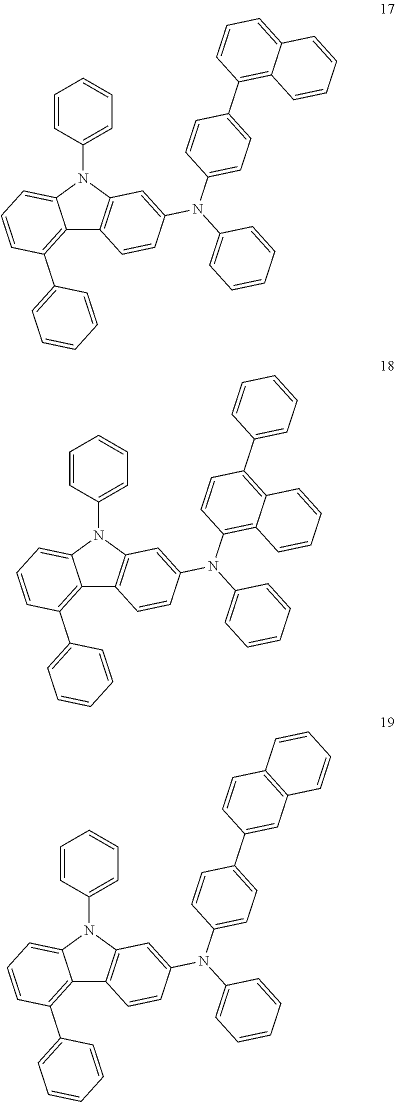 [SMILES: c1ccc(-c2cccc3c2c2ccc(N(c4ccccc4)c4ccc(-c5cccc6ccccc56)cc4)cc2n3-c2ccccc2)cc1 ; c1ccc(-c2ccc(N(c3ccccc3)c3ccc4c5c(-c6ccccc6)cccc5n(-c5ccccc5)c4c3)c3ccccc23)cc1 ; c1ccc(-c2cccc3c2c2ccc(N(c4ccccc4)c4ccc(-c5ccc6ccccc6c5)cc4)cc2n3-c2ccccc2)cc1]

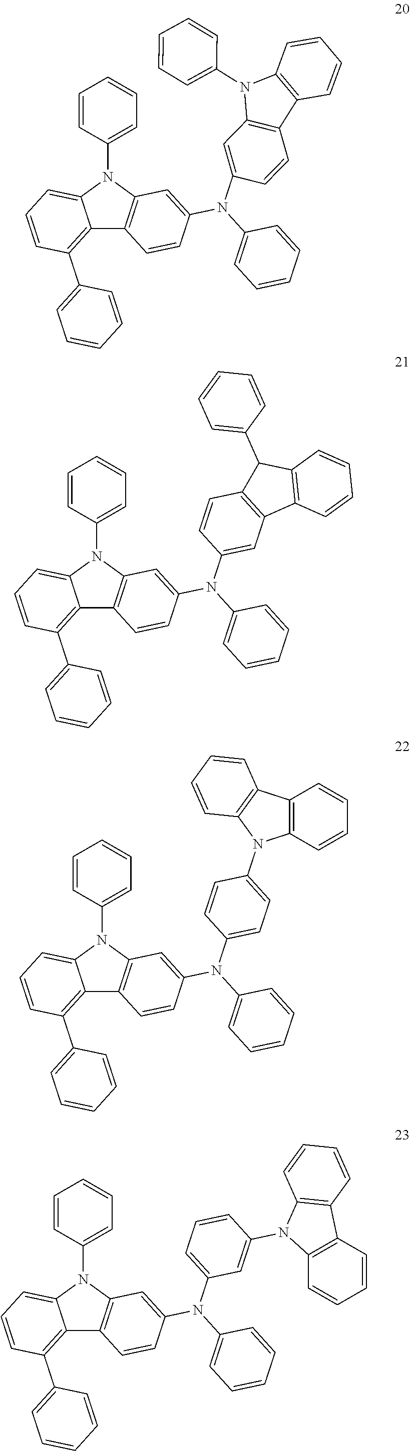 [SMILES: c1ccc(-c2cccc3c2c2ccc(N(c4ccccc4)c4ccc5c6ccccc6n(-c6ccccc6)c5c4)cc2n3-c2ccccc2)cc1 ; c1ccc(-c2cccc3c2c2ccc(N(c4ccccc4)c4ccc5c(c4)-c4ccccc4C5c4ccccc4)cc2n3-c2ccccc2)cc1 ; c1ccc(-c2cccc3c2c2ccc(N(c4ccccc4)c4ccc(-n5c6ccccc6c6ccccc65)cc4)cc2n3-c2ccccc2)cc1 ; c1ccc(-c2cccc3c2c2ccc(N(c4ccccc4)c4cccc(-n5c6ccccc6c6ccccc65)c4)cc2n3-c2ccccc2)cc1]

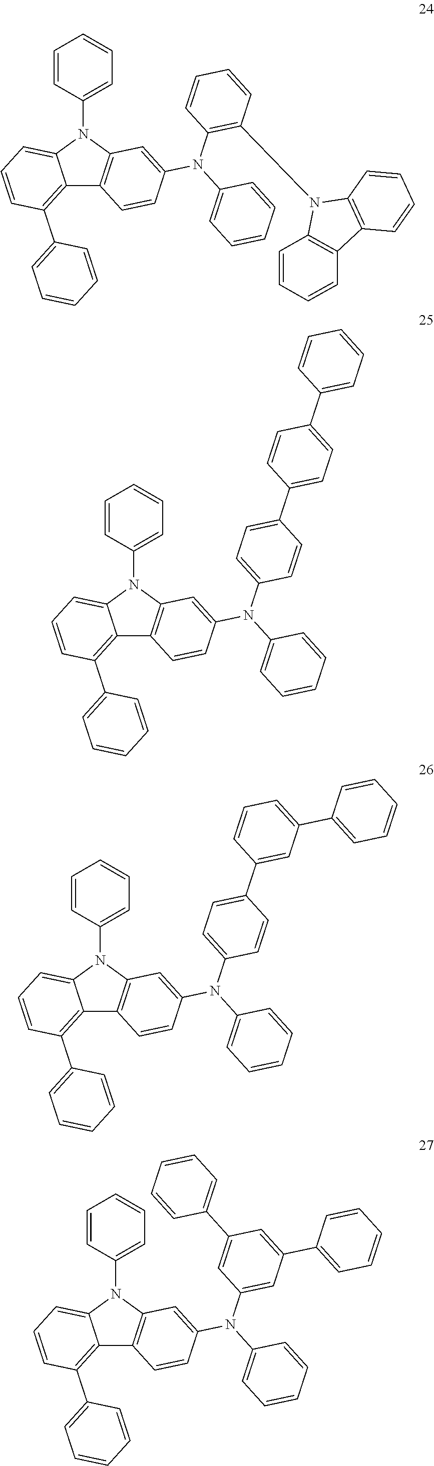

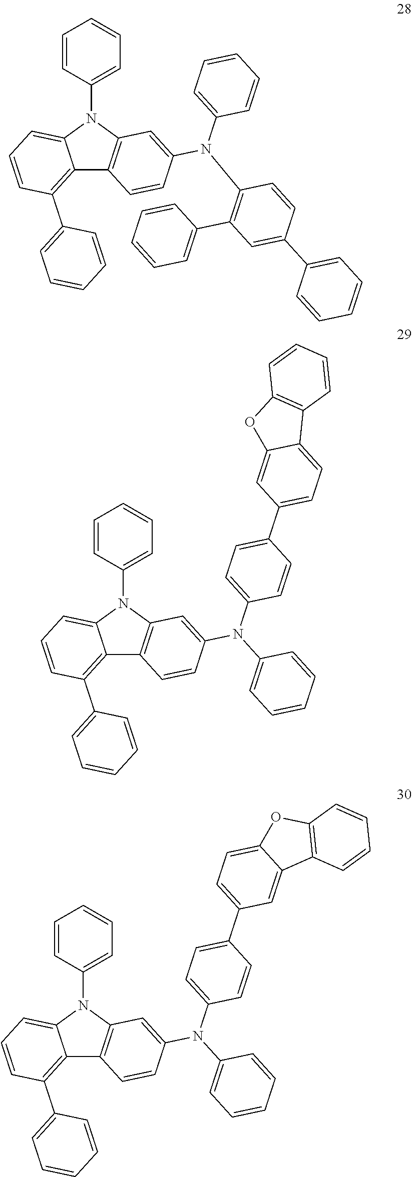

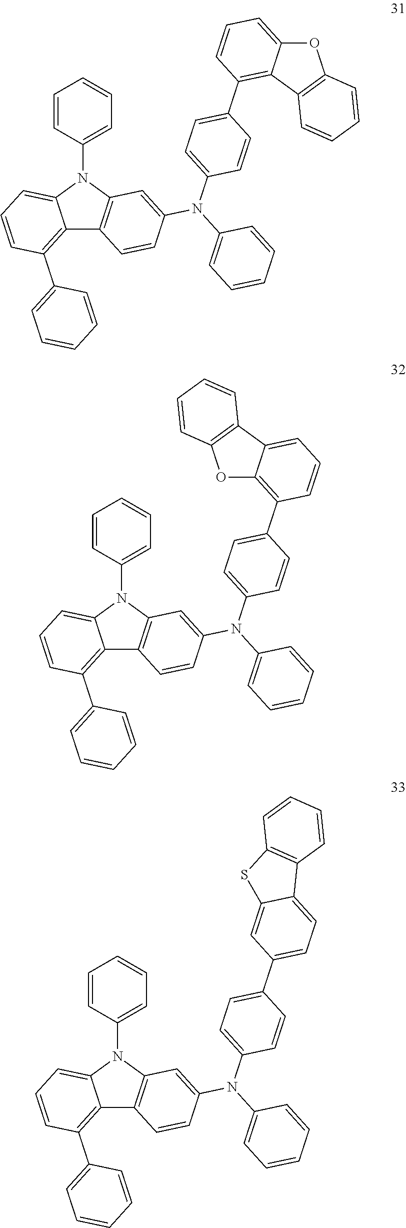 [SMILES: c1ccc(-c2cccc3c2c2ccc(N(c4ccccc4)c4ccc(-c5cccc6oc7ccccc7c56)cc4)cc2n3-c2ccccc2)cc1 ; c1ccc(-c2cccc3c2c2ccc(N(c4ccccc4)c4ccc(-c5cccc6c5oc5ccccc56)cc4)cc2n3-c2ccccc2)cc1 ; c1ccc(-c2cccc3c2c2ccc(N(c4ccccc4)c4ccc(-c5ccc6c(c5)sc5ccccc56)cc4)cc2n3-c2ccccc2)cc1]

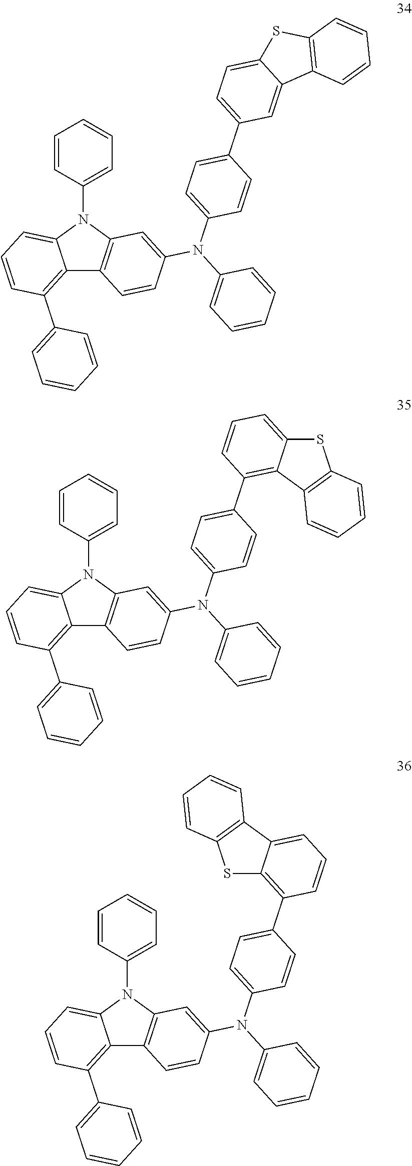 [SMILES: c1ccc(-c2cccc3c2c2ccc(N(c4ccccc4)c4ccc(-c5ccc6sc7ccccc7c6c5)cc4)cc2n3-c2ccccc2)cc1 ; c1ccc(-c2cccc3c2c2ccc(N(c4ccccc4)c4ccc(-c5cccc6sc7ccccc7c56)cc4)cc2n3-c2ccccc2)cc1 ; c1ccc(-c2cccc3c2c2ccc(N(c4ccccc4)c4ccc(-c5cccc6c5sc5ccccc56)cc4)cc2n3-c2ccccc2)cc1]

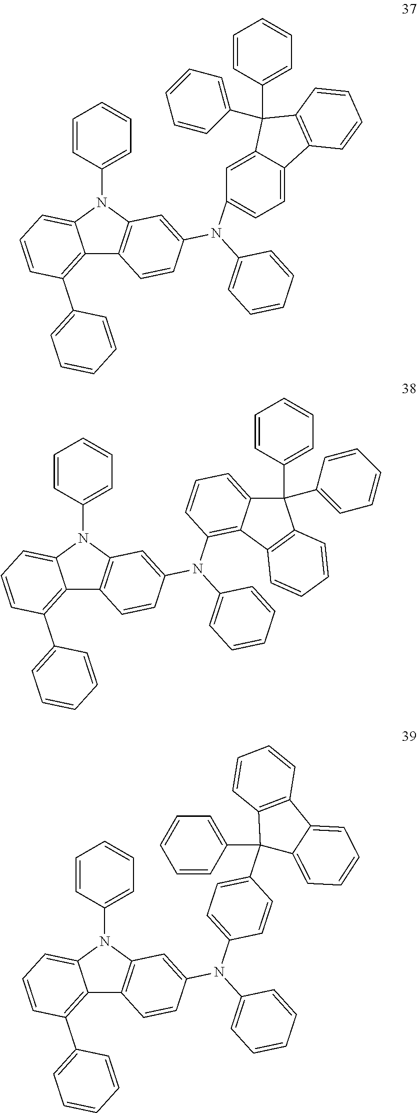 [SMILES: c1ccc(-c2cccc3c2c2ccc(N(c4ccccc4)c4ccc5c(c4)C(c4ccccc4)(c4ccccc4)c4ccccc4-5)cc2n3-c2ccccc2)cc1 ; c1ccc(-c2cccc3c2c2ccc(N(c4ccccc4)c4cccc5c4-c4ccccc4C5(c4ccccc4)c4ccccc4)cc2n3-c2ccccc2)cc1 ; c1ccc(-c2cccc3c2c2ccc(N(c4ccccc4)c4ccc(C5(c6ccccc6)c6ccccc6-c6ccccc65)cc4)cc2n3-c2ccccc2)cc1]

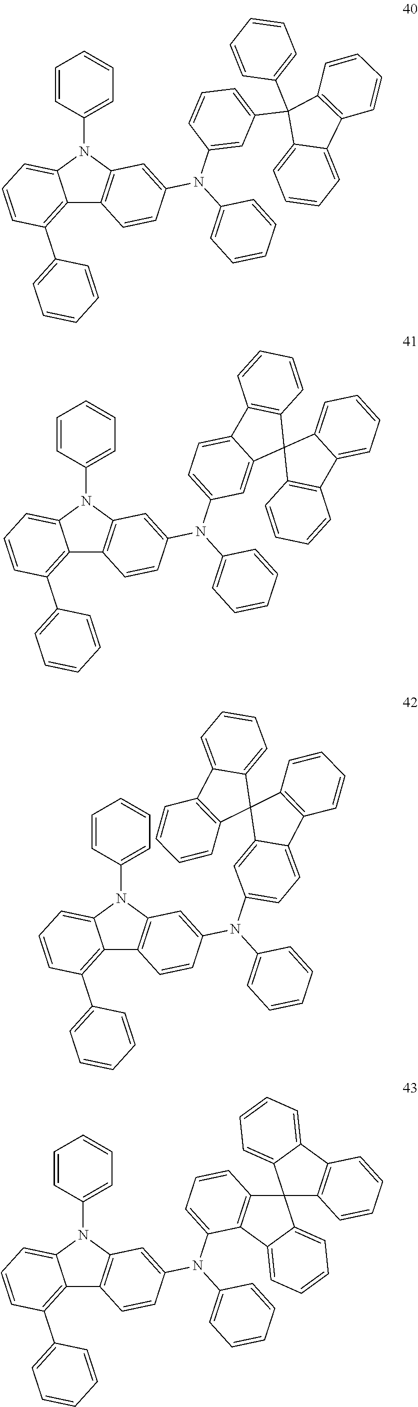 [SMILES: c1ccc(-c2cccc3c2c2ccc(N(c4ccccc4)c4cccc(C5(c6ccccc6)c6ccccc6-c6ccccc65)c4)cc2n3-c2ccccc2)cc1 ; c1ccc(-c2cccc3c2c2ccc(N(c4ccccc4)c4ccc5c(c4)C4(c6ccccc6-c6ccccc64)c4ccccc4-5)cc2n3-c2ccccc2)cc1 ; c1ccc(-c2cccc3c2c2ccc(N(c4ccccc4)c4ccc5c(c4)C4(c6ccccc6-c6ccccc64)c4ccccc4-5)cc2n3-c2ccccc2)cc1 ; c1ccc(-c2cccc3c2c2ccc(N(c4ccccc4)c4cccc5c4-c4ccccc4C54c5ccccc5-c5ccccc54)cc2n3-c2ccccc2)cc1]

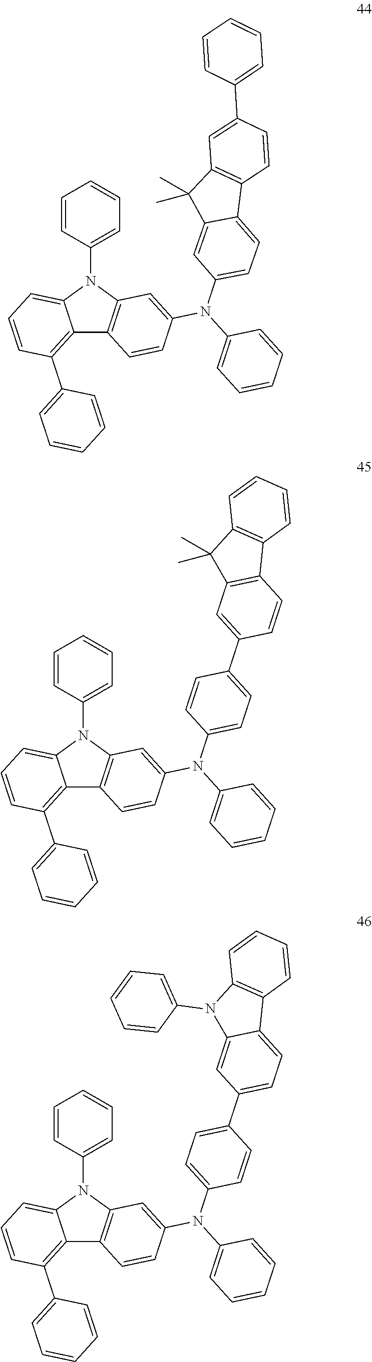 [SMILES: CC1(C)c2cc(-c3ccccc3)ccc2-c2ccc(N(c3ccccc3)c3ccc4c5c(-c6ccccc6)cccc5n(-c5ccccc5)c4c3)cc21 ; CC1(C)c2ccccc2-c2ccc(-c3ccc(N(c4ccccc4)c4ccc5c6c(-c7ccccc7)cccc6n(-c6ccccc6)c5c4)cc3)cc21 ; c1ccc(-c2cccc3c2c2ccc(N(c4ccccc4)c4ccc(-c5ccc6c7ccccc7n(-c7ccccc7)c6c5)cc4)cc2n3-c2ccccc2)cc1]

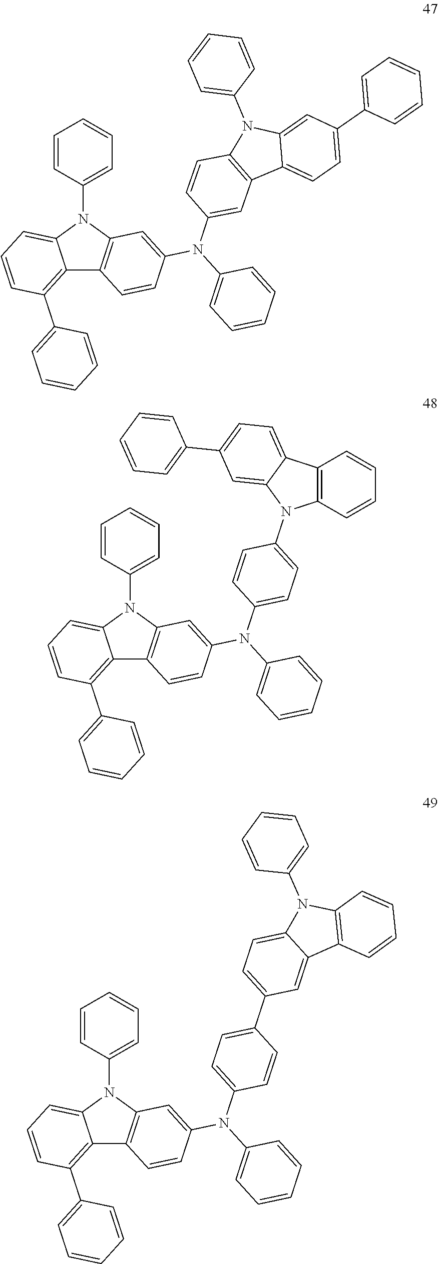 [SMILES: c1ccc(-c2ccc3c4cc(N(c5ccccc5)c5ccc6c7c(-c8ccccc8)cccc7n(-c7ccccc7)c6c5)ccc4n(-c4ccccc4)c3c2)cc1 ; c1ccc(-c2ccc3c4ccccc4n(-c4ccc(N(c5ccccc5)c5ccc6c7c(-c8ccccc8)cccc7n(-c7ccccc7)c6c5)cc4)c3c2)cc1 ; c1ccc(-c2cccc3c2c2ccc(N(c4ccccc4)c4ccc(-c5ccc6c(c5)c5ccccc5n6-c5ccccc5)cc4)cc2n3-c2ccccc2)cc1]

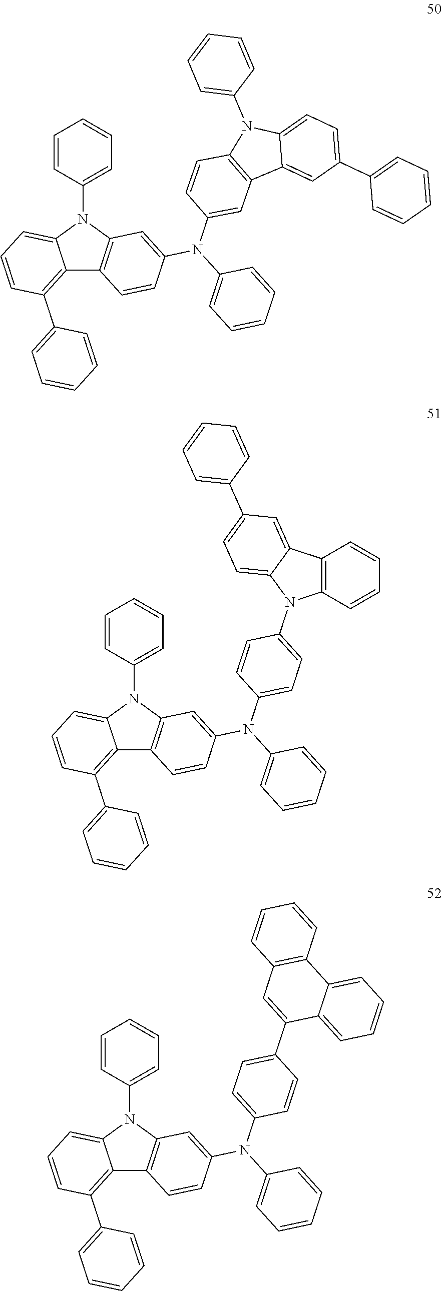 [SMILES: c1ccc(-c2ccc3c(c2)c2cc(N(c4ccccc4)c4ccc5c6c(-c7ccccc7)cccc6n(-c6ccccc6)c5c4)ccc2n3-c2ccccc2)cc1 ; c1ccc(-c2ccc3c(c2)c2ccccc2n3-c2ccc(N(c3ccccc3)c3ccc4c5c(-c6ccccc6)cccc5n(-c5ccccc5)c4c3)cc2)cc1 ; c1ccc(-c2cccc3c2c2ccc(N(c4ccccc4)c4ccc(-c5cc6ccccc6c6ccccc56)cc4)cc2n3-c2ccccc2)cc1]

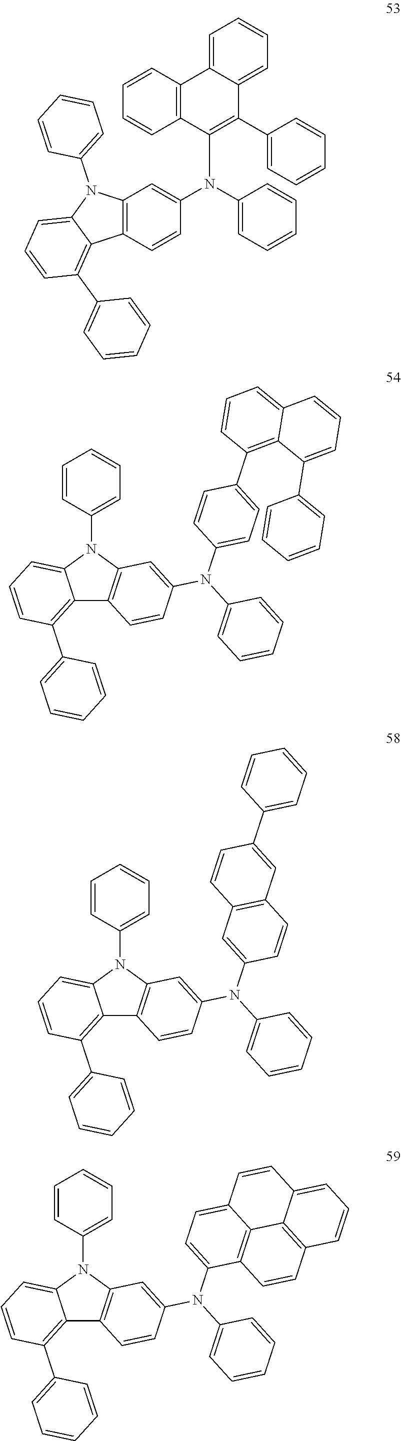

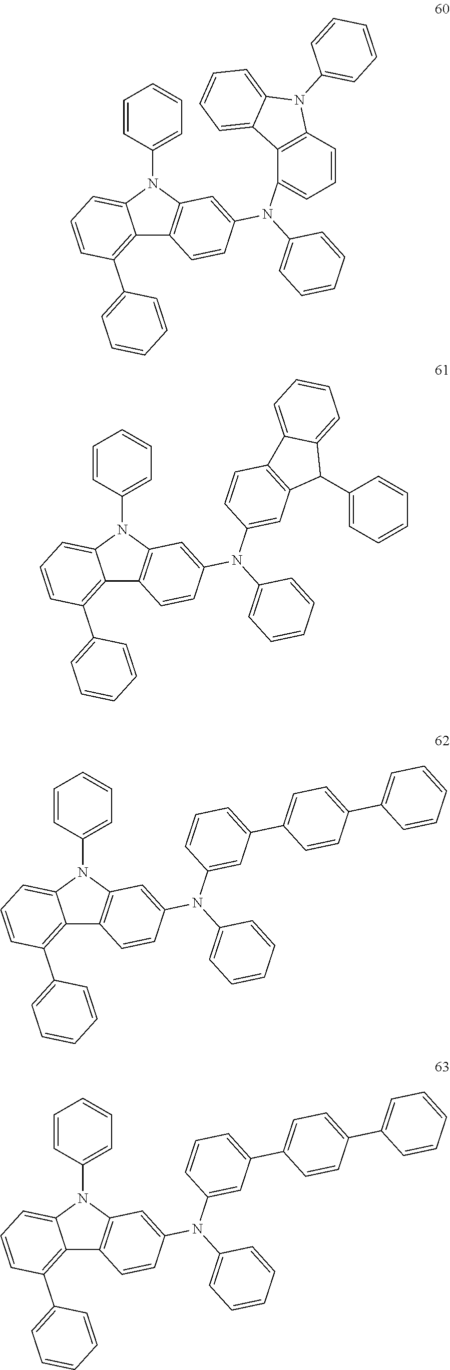 [SMILES: c1ccc(-c2cccc3c2c2ccc(N(c4ccccc4)c4cccc5c4c4ccccc4n5-c4ccccc4)cc2n3-c2ccccc2)cc1 ; c1ccc(-c2cccc3c2c2ccc(N(c4ccccc4)c4ccc5c(c4)C(c4ccccc4)c4ccccc4-5)cc2n3-c2ccccc2)cc1 ; c1ccc(-c2ccc(-c3cccc(N(c4ccccc4)c4ccc5c6c(-c7ccccc7)cccc6n(-c6ccccc6)c5c4)c3)cc2)cc1 ; c1ccc(-c2ccc(-c3cccc(N(c4ccccc4)c4ccc5c6c(-c7ccccc7)cccc6n(-c6ccccc6)c5c4)c3)cc2)cc1]

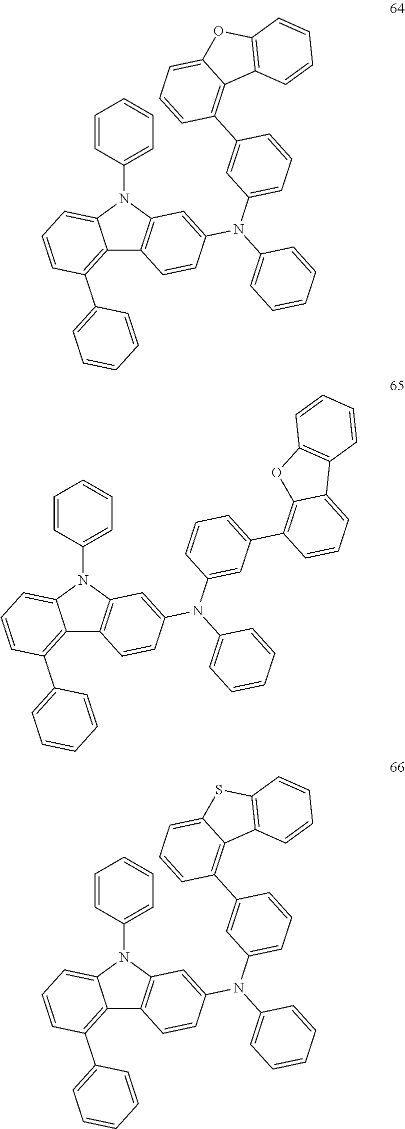 [SMILES: c1ccc(-c2cccc3c2c2ccc(N(c4ccccc4)c4cccc(-c5cccc6oc7ccccc7c56)c4)cc2n3-c2ccccc2)cc1 ; c1ccc(-c2cccc3c2c2ccc(N(c4ccccc4)c4cccc(-c5cccc6c5oc5ccccc56)c4)cc2n3-c2ccccc2)cc1 ; c1ccc(-c2cccc3c2c2ccc(N(c4ccccc4)c4cccc(-c5cccc6sc7ccccc7c56)c4)cc2n3-c2ccccc2)cc1]

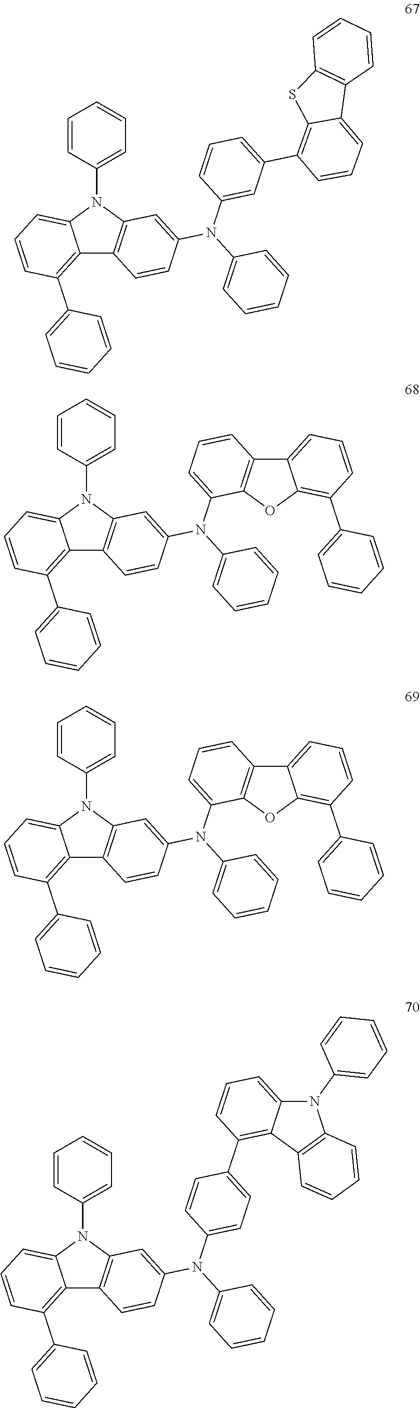

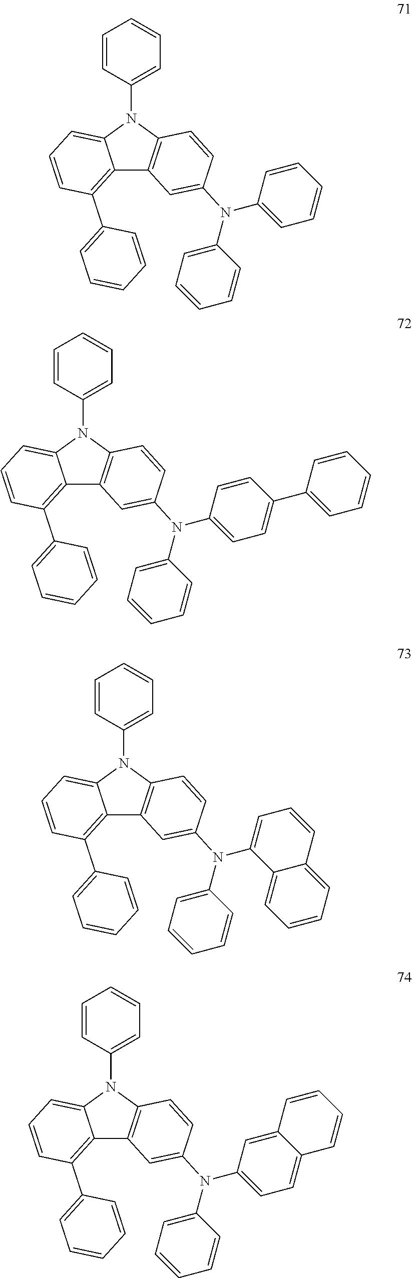 [SMILES: c1ccc(-c2cccc3c2c2cc(N(c4ccccc4)c4ccccc4)ccc2n3-c2ccccc2)cc1 ; c1ccc(-c2ccc(N(c3ccccc3)c3ccc4c(c3)c3c(-c5ccccc5)cccc3n4-c3ccccc3)cc2)cc1 ; c1ccc(-c2cccc3c2c2cc(N(c4ccccc4)c4cccc5ccccc45)ccc2n3-c2ccccc2)cc1 ; c1ccc(-c2cccc3c2c2cc(N(c4ccccc4)c4ccc5ccccc5c4)ccc2n3-c2ccccc2)cc1]

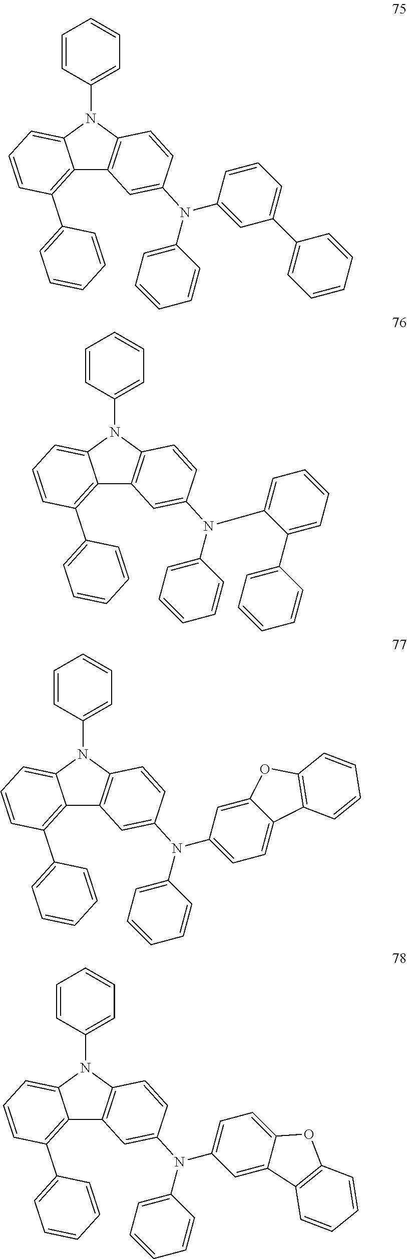

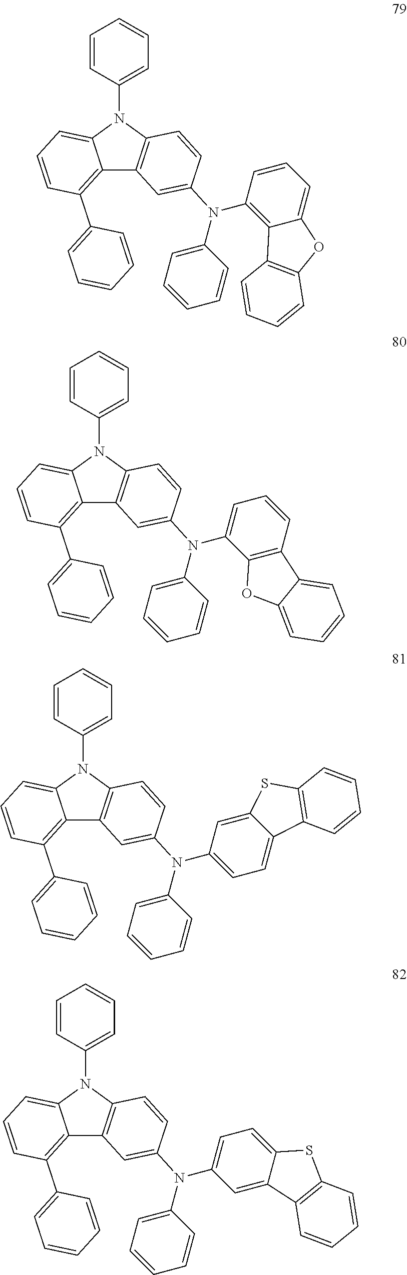

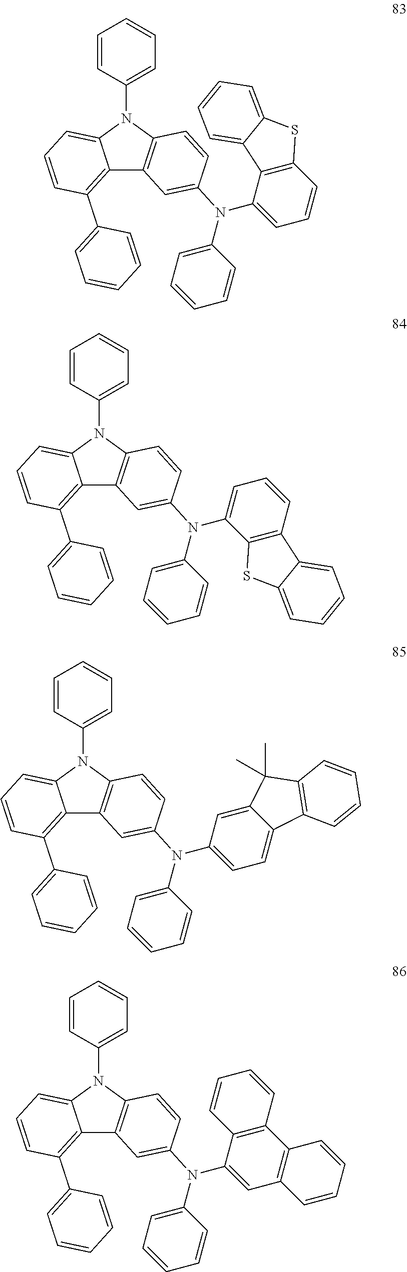 [SMILES: c1ccc(-c2cccc3c2c2cc(N(c4ccccc4)c4cccc5sc6ccccc6c45)ccc2n3-c2ccccc2)cc1 ; c1ccc(-c2cccc3c2c2cc(N(c4ccccc4)c4cccc5c4sc4ccccc45)ccc2n3-c2ccccc2)cc1 ; CC1(C)c2ccccc2-c2ccc(N(c3ccccc3)c3ccc4c(c3)c3c(-c5ccccc5)cccc3n4-c3ccccc3)cc21 ; c1ccc(-c2cccc3c2c2cc(N(c4ccccc4)c4cc5ccccc5c5ccccc45)ccc2n3-c2ccccc2)cc1]

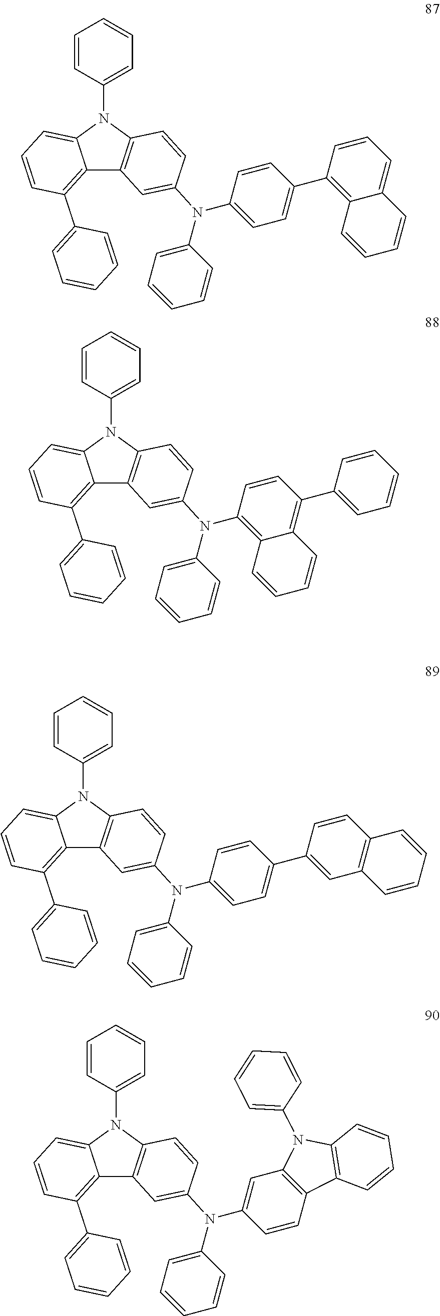

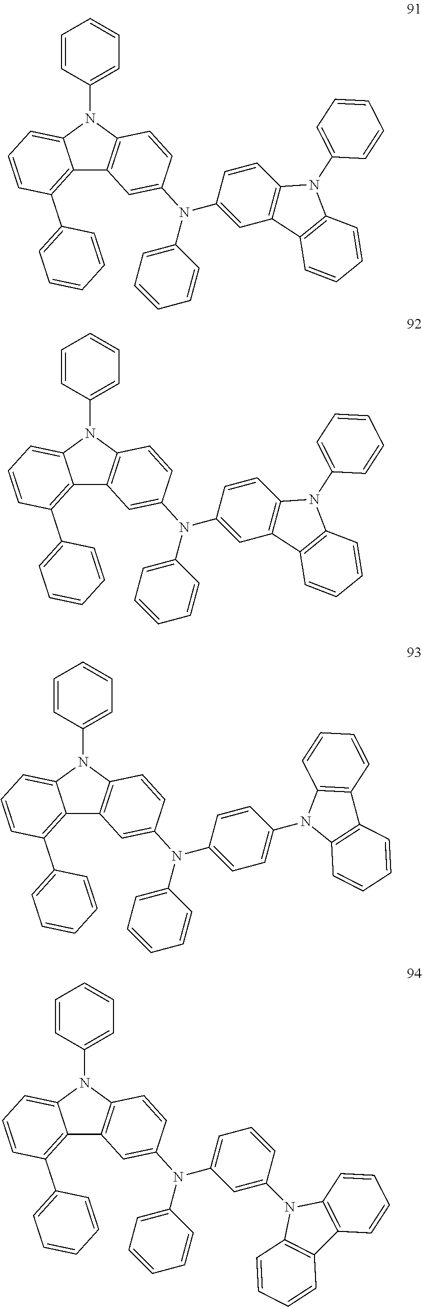

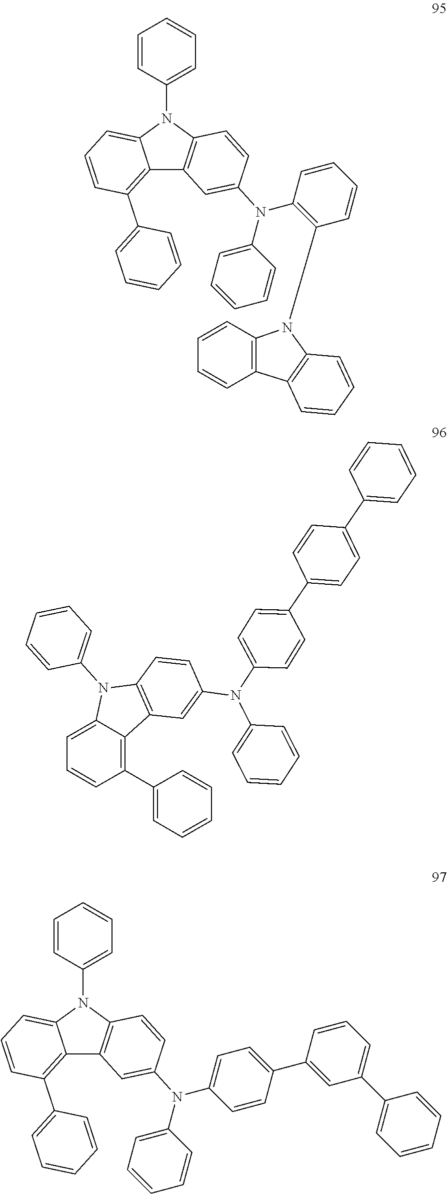

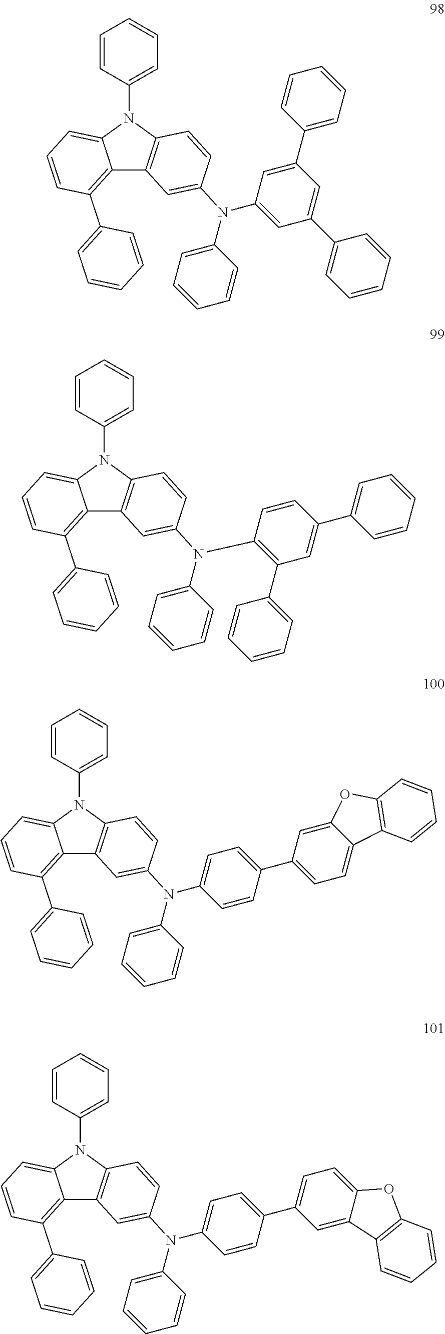 [SMILES: c1ccc(-c2cc(-c3ccccc3)cc(N(c3ccccc3)c3ccc4c(c3)c3c(-c5ccccc5)cccc3n4-c3ccccc3)c2)cc1 ; c1ccc(-c2ccc(N(c3ccccc3)c3ccc4c(c3)c3c(-c5ccccc5)cccc3n4-c3ccccc3)c(-c3ccccc3)c2)cc1 ; c1ccc(-c2cccc3c2c2cc(N(c4ccccc4)c4ccc(-c5ccc6c(c5)oc5ccccc56)cc4)ccc2n3-c2ccccc2)cc1 ; c1ccc(-c2cccc3c2c2cc(N(c4ccccc4)c4ccc(-c5ccc6oc7ccccc7c6c5)cc4)ccc2n3-c2ccccc2)cc1]

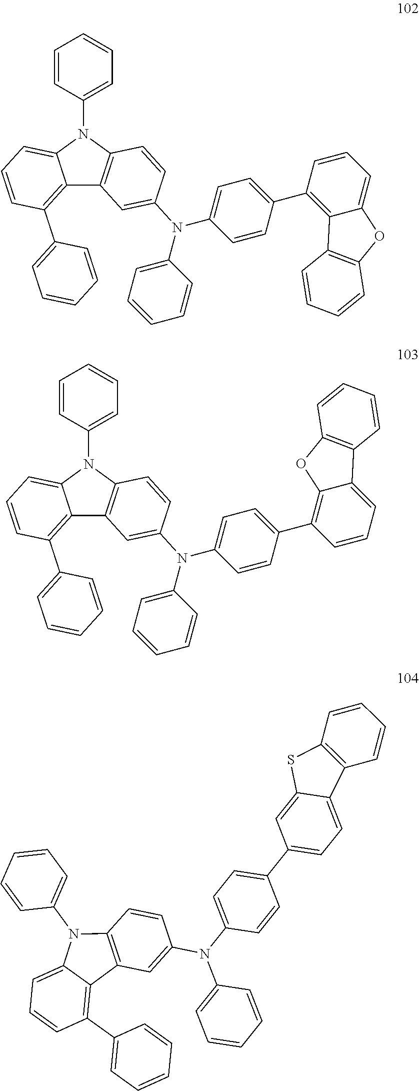 [SMILES: c1ccc(-c2cccc3c2c2cc(N(c4ccccc4)c4ccc(-c5cccc6oc7ccccc7c56)cc4)ccc2n3-c2ccccc2)cc1 ; c1ccc(-c2cccc3c2c2cc(N(c4ccccc4)c4ccc(-c5cccc6c5oc5ccccc56)cc4)ccc2n3-c2ccccc2)cc1 ; c1ccc(-c2cccc3c2c2cc(N(c4ccccc4)c4ccc(-c5ccc6c(c5)sc5ccccc56)cc4)ccc2n3-c2ccccc2)cc1]

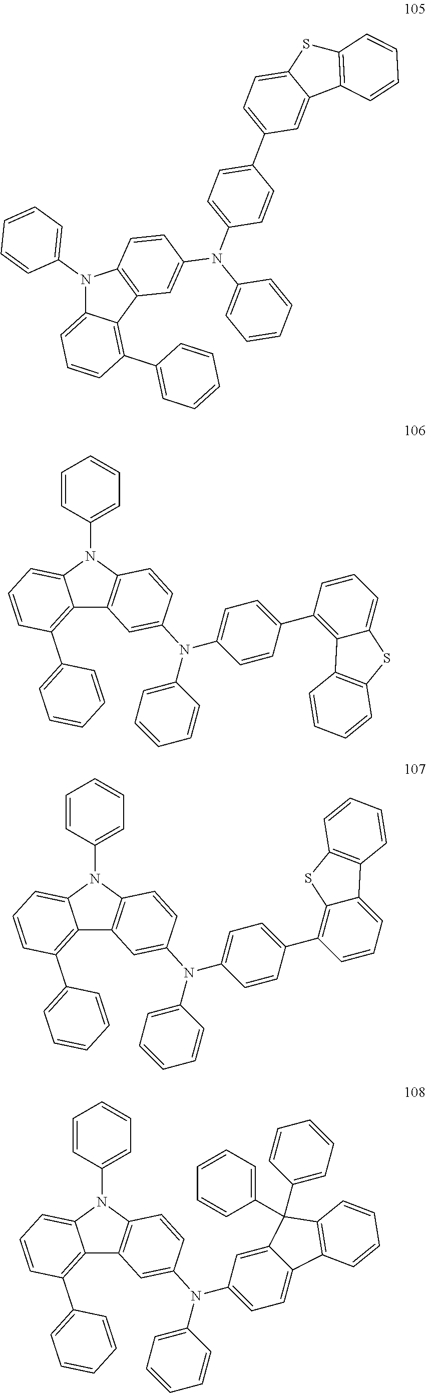 [SMILES: c1ccc(-c2cccc3c2c2cc(N(c4ccccc4)c4ccc(-c5ccc6sc7ccccc7c6c5)cc4)ccc2n3-c2ccccc2)cc1 ; c1ccc(-c2cccc3c2c2cc(N(c4ccccc4)c4ccc(-c5cccc6sc7ccccc7c56)cc4)ccc2n3-c2ccccc2)cc1 ; c1ccc(-c2cccc3c2c2cc(N(c4ccccc4)c4ccc(-c5cccc6c5sc5ccccc56)cc4)ccc2n3-c2ccccc2)cc1 ; c1ccc(-c2cccc3c2c2cc(N(c4ccccc4)c4ccc5c(c4)C(c4ccccc4)(c4ccccc4)c4ccccc4-5)ccc2n3-c2ccccc2)cc1]

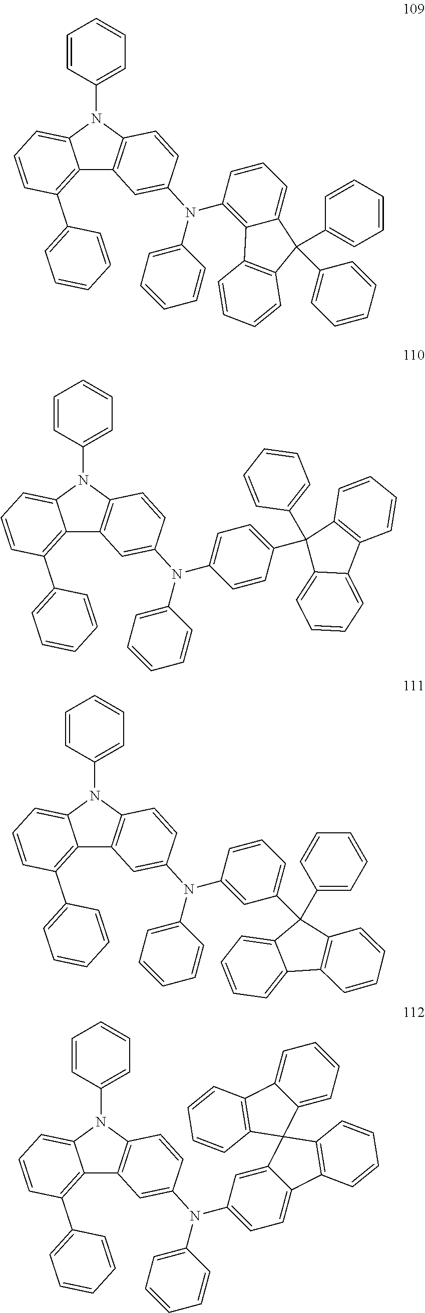 [SMILES: c1ccc(-c2cccc3c2c2cc(N(c4ccccc4)c4cccc5c4-c4ccccc4C5(c4ccccc4)c4ccccc4)ccc2n3-c2ccccc2)cc1 ; c1ccc(-c2cccc3c2c2cc(N(c4ccccc4)c4ccc(C5(c6ccccc6)c6ccccc6-c6ccccc65)cc4)ccc2n3-c2ccccc2)cc1 ; c1ccc(-c2cccc3c2c2cc(N(c4ccccc4)c4cccc(C5(c6ccccc6)c6ccccc6-c6ccccc65)c4)ccc2n3-c2ccccc2)cc1 ; c1ccc(-c2cccc3c2c2cc(N(c4ccccc4)c4ccc5c(c4)C4(c6ccccc6-c6ccccc64)c4ccccc4-5)ccc2n3-c2ccccc2)cc1]

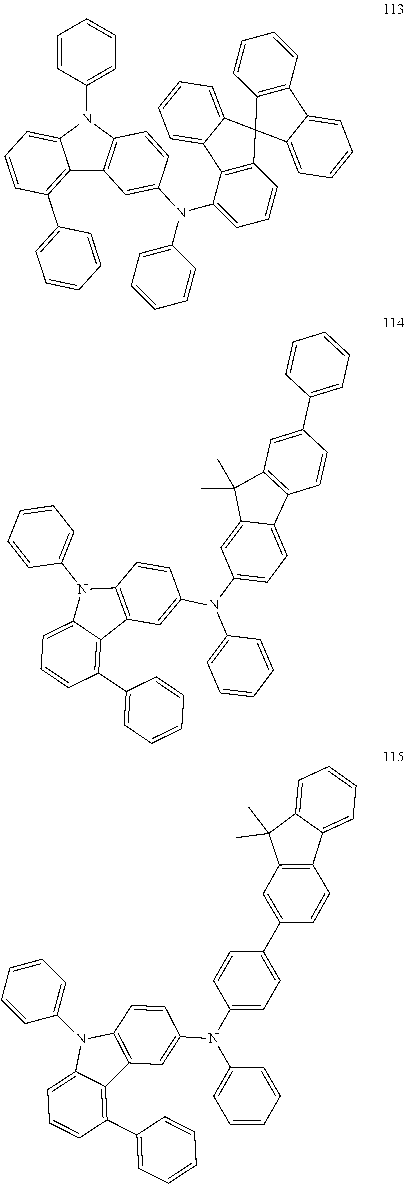 [SMILES: c1ccc(-c2cccc3c2c2cc(N(c4ccccc4)c4cccc5c4-c4ccccc4C54c5ccccc5-c5ccccc54)ccc2n3-c2ccccc2)cc1 ; CC1(C)c2cc(-c3ccccc3)ccc2-c2ccc(N(c3ccccc3)c3ccc4c(c3)c3c(-c5ccccc5)cccc3n4-c3ccccc3)cc21 ; CC1(C)c2ccccc2-c2ccc(-c3ccc(N(c4ccccc4)c4ccc5c(c4)c4c(-c6ccccc6)cccc4n5-c4ccccc4)cc3)cc21]

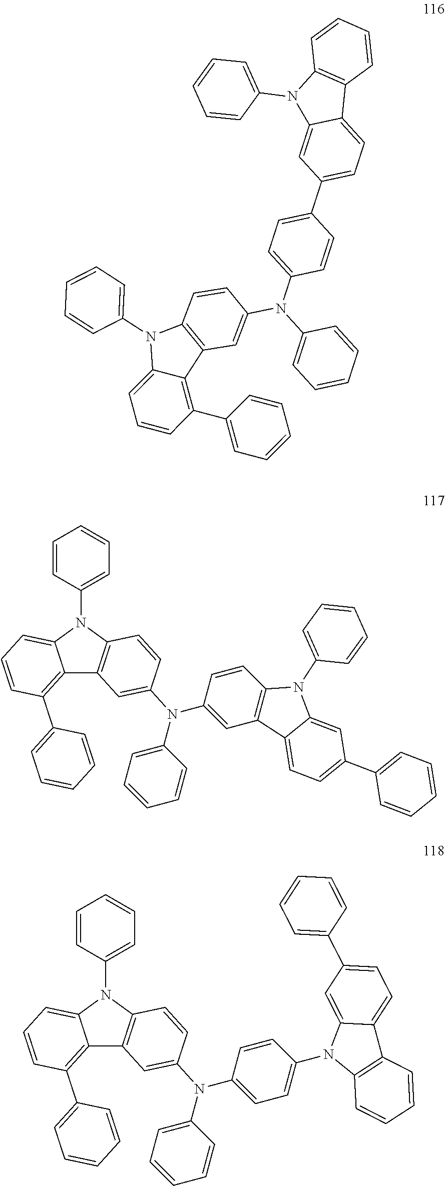 [SMILES: c1ccc(-c2cccc3c2c2cc(N(c4ccccc4)c4ccc(-c5ccc6c7ccccc7n(-c7ccccc7)c6c5)cc4)ccc2n3-c2ccccc2)cc1 ; c1ccc(-c2ccc3c4cc(N(c5ccccc5)c5ccc6c(c5)c5c(-c7ccccc7)cccc5n6-c5ccccc5)ccc4n(-c4ccccc4)c3c2)cc1 ; c1ccc(-c2ccc3c4ccccc4n(-c4ccc(N(c5ccccc5)c5ccc6c(c5)c5c(-c7ccccc7)cccc5n6-c5ccccc5)cc4)c3c2)cc1]

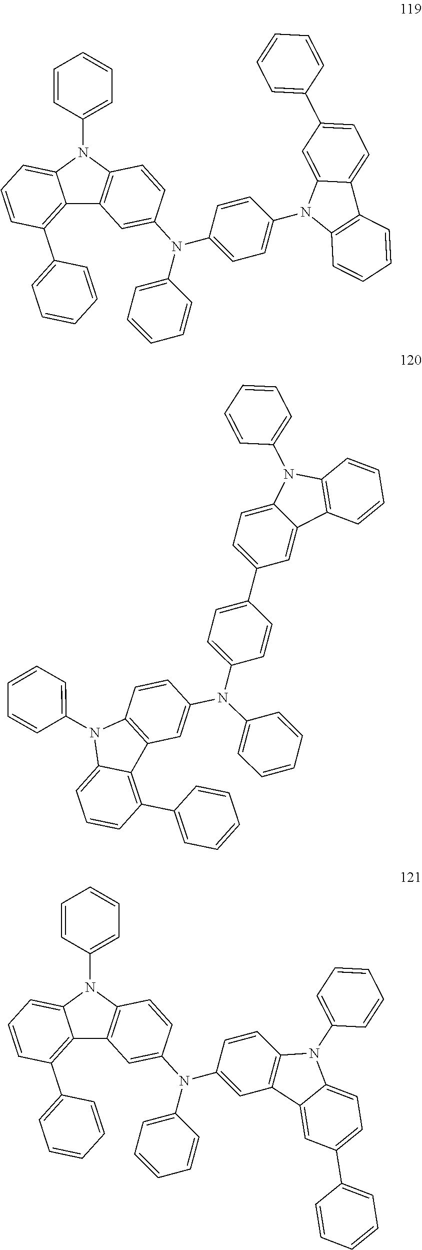 [SMILES: c1ccc(-c2ccc3c4ccccc4n(-c4ccc(N(c5ccccc5)c5ccc6c(c5)c5c(-c7ccccc7)cccc5n6-c5ccccc5)cc4)c3c2)cc1 ; c1ccc(-c2cccc3c2c2cc(N(c4ccccc4)c4ccc(-c5ccc6c(c5)c5ccccc5n6-c5ccccc5)cc4)ccc2n3-c2ccccc2)cc1 ; c1ccc(-c2ccc3c(c2)c2cc(N(c4ccccc4)c4ccc5c(c4)c4c(-c6ccccc6)cccc4n5-c4ccccc4)ccc2n3-c2ccccc2)cc1]

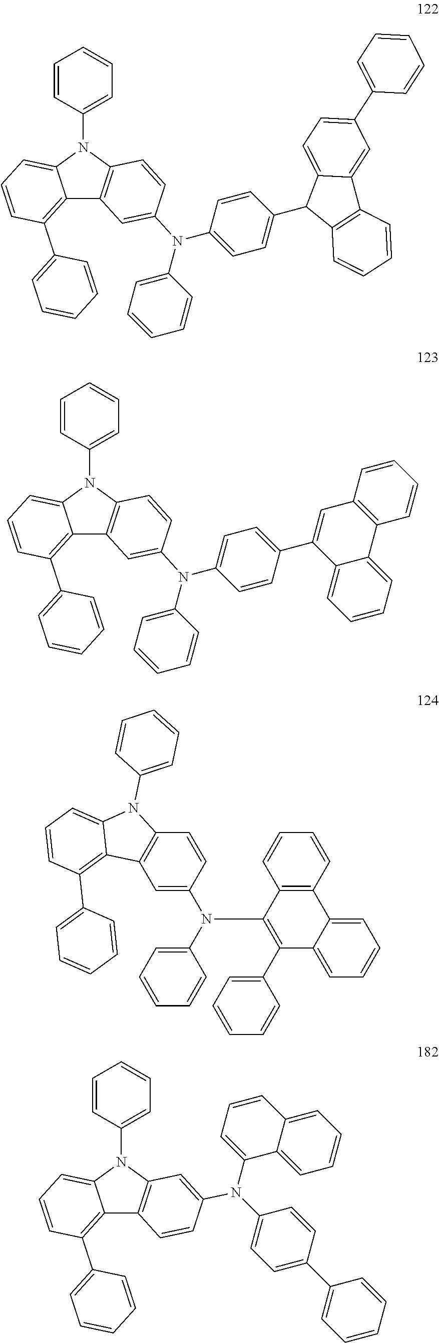

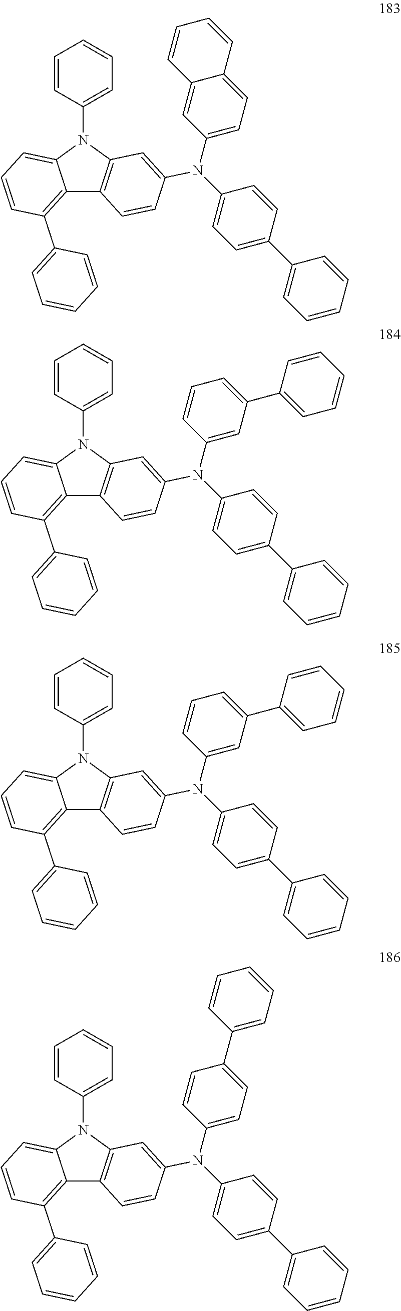

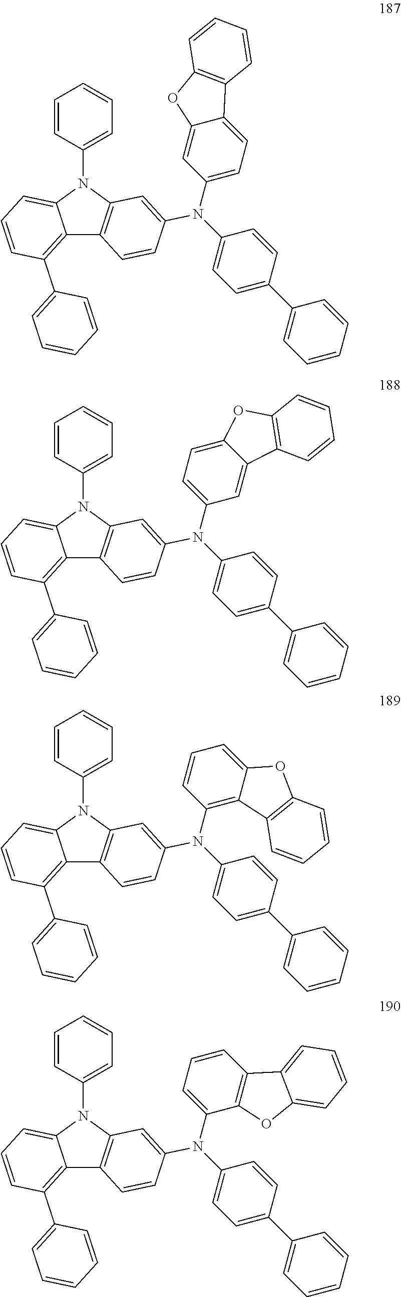

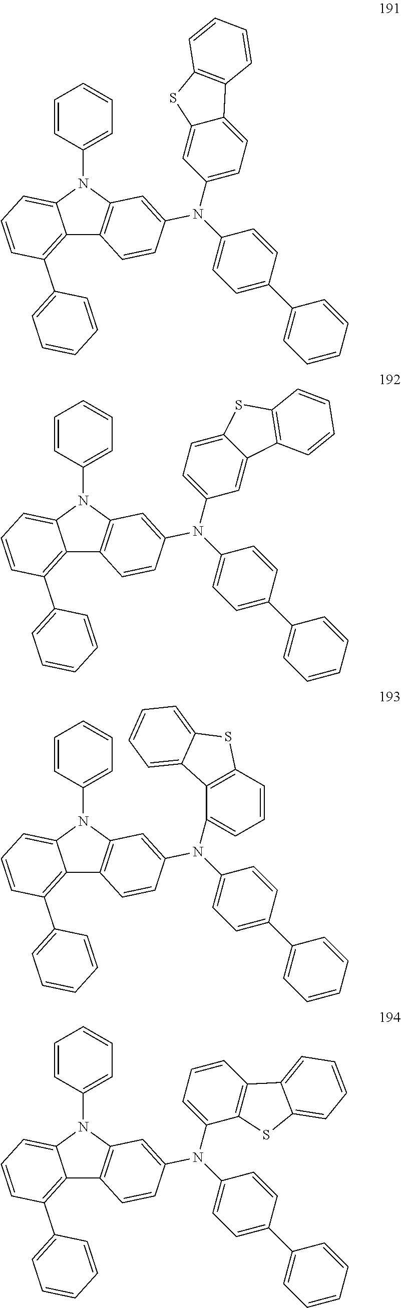

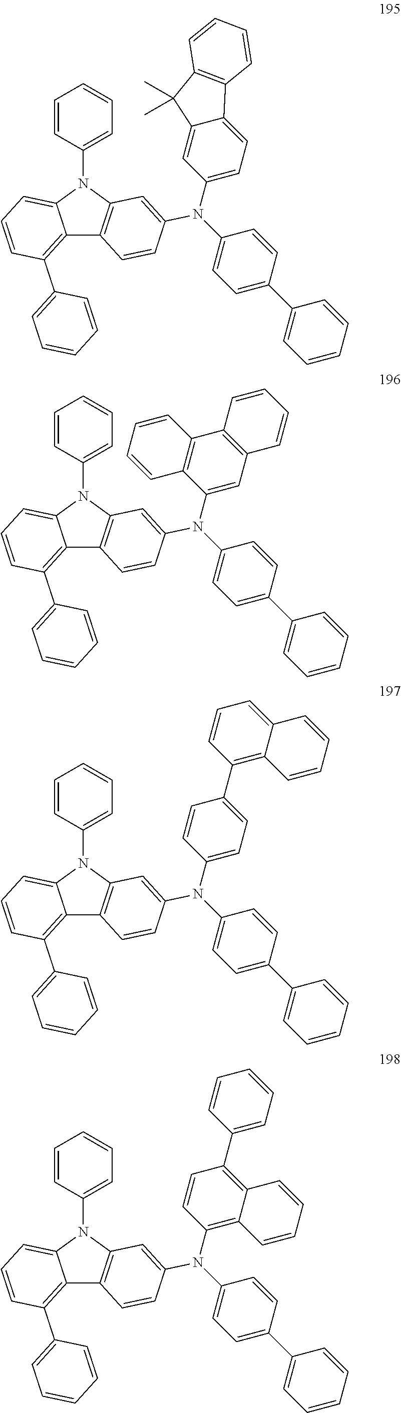

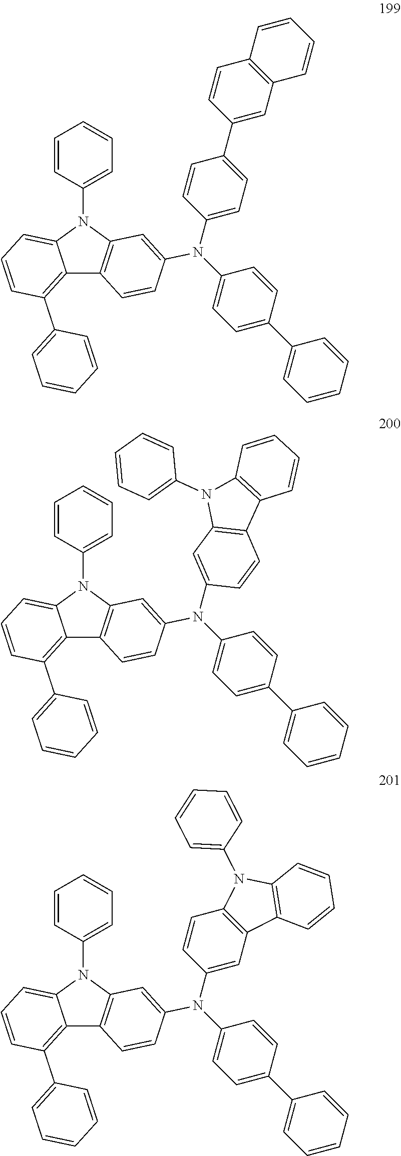

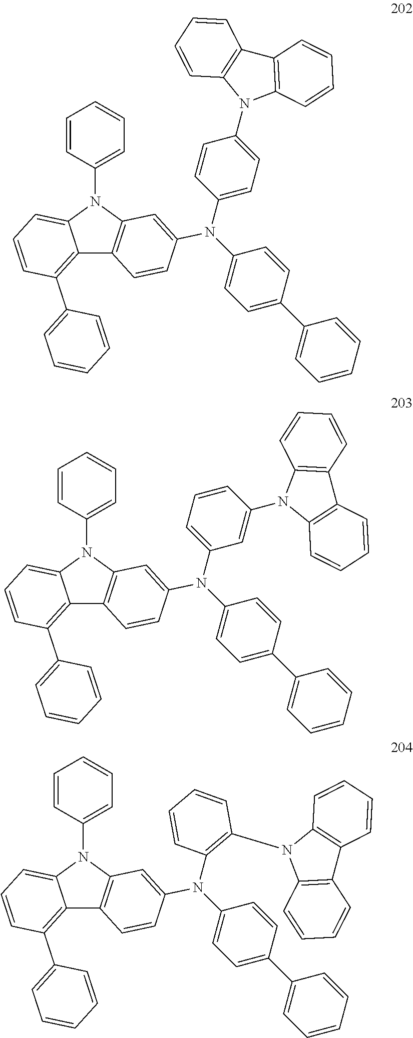

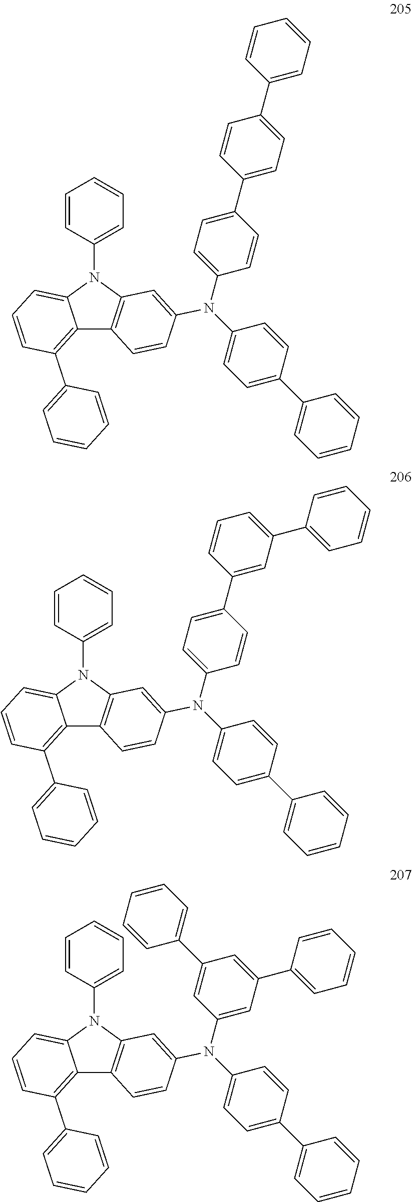

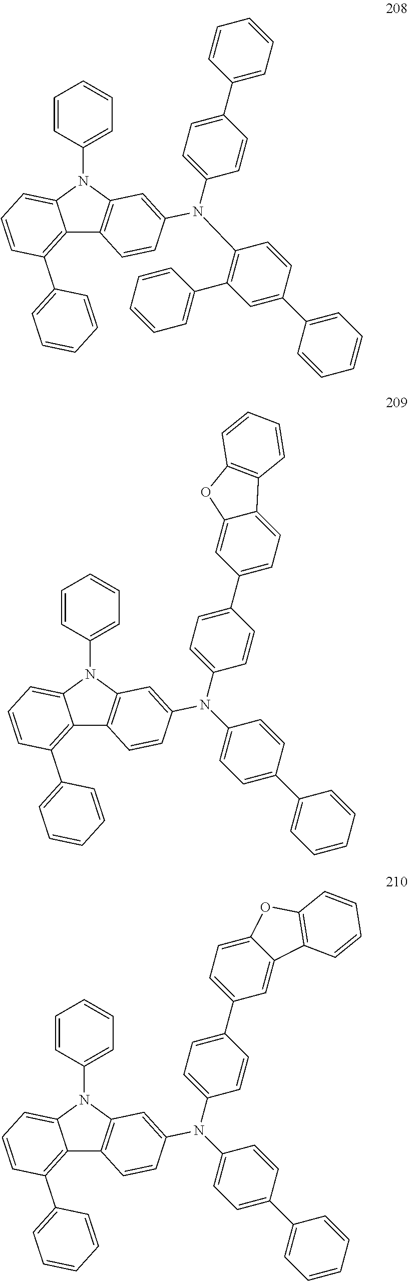

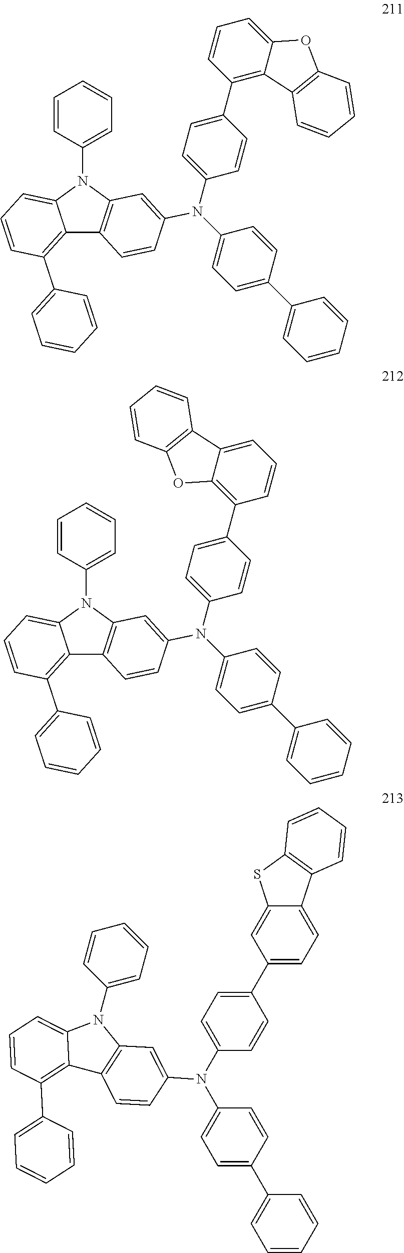

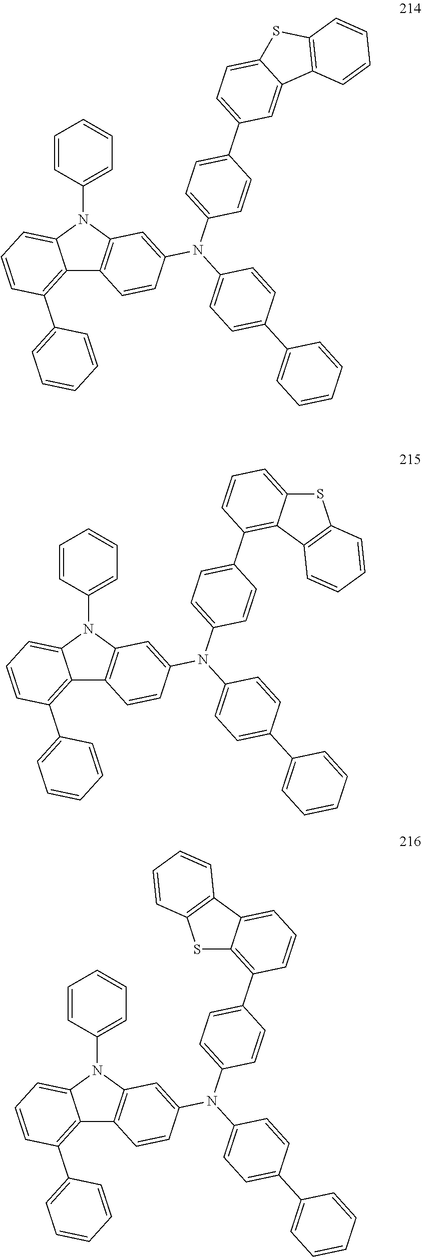 [SMILES: c1ccc(-c2ccc(N(c3ccc(-c4ccc5sc6ccccc6c5c4)cc3)c3ccc4c5c(-c6ccccc6)cccc5n(-c5ccccc5)c4c3)cc2)cc1 ; c1ccc(-c2ccc(N(c3ccc(-c4cccc5sc6ccccc6c45)cc3)c3ccc4c5c(-c6ccccc6)cccc5n(-c5ccccc5)c4c3)cc2)cc1 ; c1ccc(-c2ccc(N(c3ccc(-c4cccc5c4sc4ccccc45)cc3)c3ccc4c5c(-c6ccccc6)cccc5n(-c5ccccc5)c4c3)cc2)cc1]

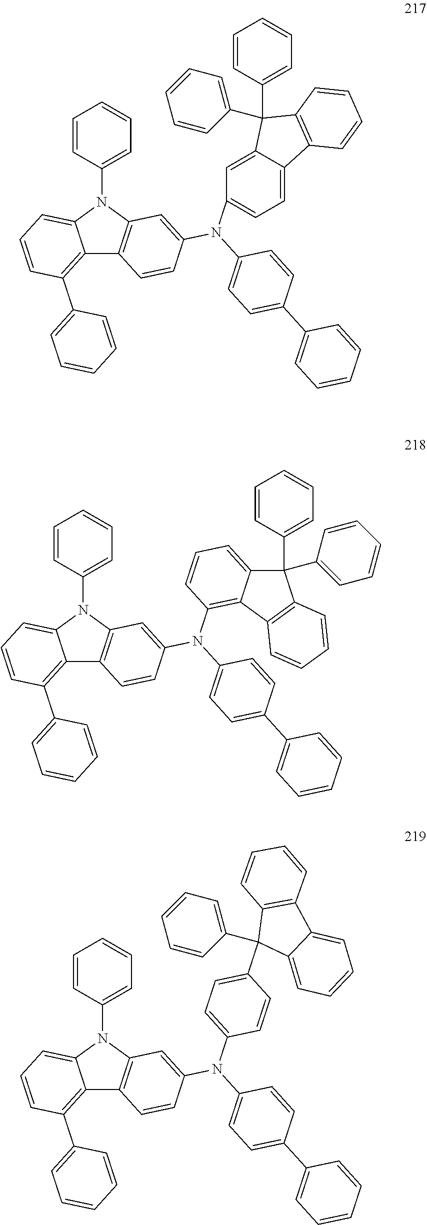 [SMILES: c1ccc(-c2ccc(N(c3ccc4c(c3)C(c3ccccc3)(c3ccccc3)c3ccccc3-4)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3cccc4c3-c3ccccc3C4(c3ccccc3)c3ccccc3)cc2)cc1 ; c1ccc(-c2ccc(N(c3ccc(C4(c5ccccc5)c5ccccc5-c5ccccc54)cc3)c3ccc4c5c(-c6ccccc6)cccc5n(-c5ccccc5)c4c3)cc2)cc1]

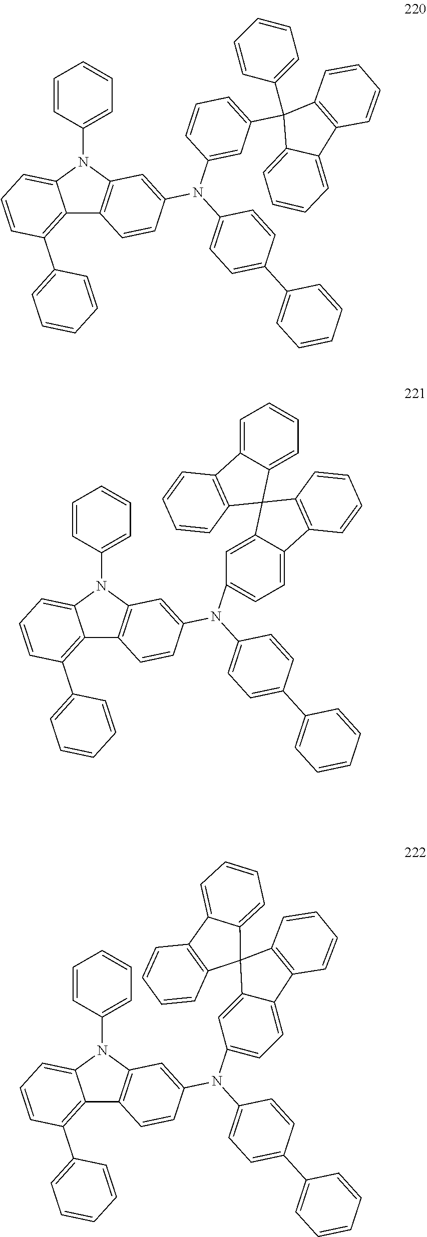 [SMILES: c1ccc(-c2ccc(N(c3cccc(C4(c5ccccc5)c5ccccc5-c5ccccc54)c3)c3ccc4c5c(-c6ccccc6)cccc5n(-c5ccccc5)c4c3)cc2)cc1 ; c1ccc(-c2ccc(N(c3ccc4c(c3)C3(c5ccccc5-c5ccccc53)c3ccccc3-4)c3ccc4c5c(-c6ccccc6)cccc5n(-c5ccccc5)c4c3)cc2)cc1 ; c1ccc(-c2ccc(N(c3ccc4c(c3)C3(c5ccccc5-c5ccccc53)c3ccccc3-4)c3ccc4c5c(-c6ccccc6)cccc5n(-c5ccccc5)c4c3)cc2)cc1]

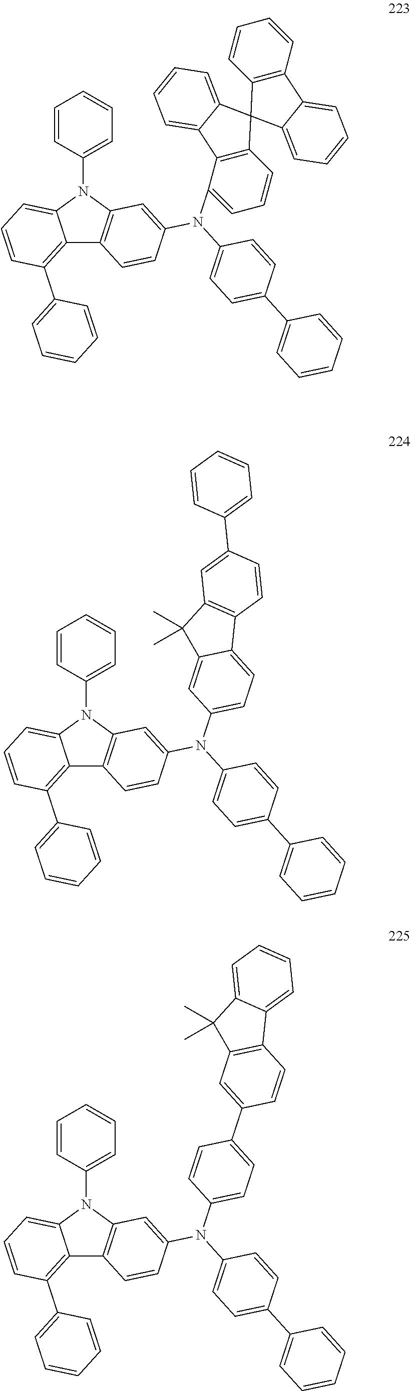 [SMILES: c1ccc(-c2ccc(N(c3ccc4c5c(-c6ccccc6)cccc5n(-c5ccccc5)c4c3)c3cccc4c3-c3ccccc3C43c4ccccc4-c4ccccc43)cc2)cc1 ; CC1(C)c2cc(-c3ccccc3)ccc2-c2ccc(N(c3ccc(-c4ccccc4)cc3)c3ccc4c5c(-c6ccccc6)cccc5n(-c5ccccc5)c4c3)cc21 ; CC1(C)c2ccccc2-c2ccc(-c3ccc(N(c4ccc(-c5ccccc5)cc4)c4ccc5c6c(-c7ccccc7)cccc6n(-c6ccccc6)c5c4)cc3)cc21]

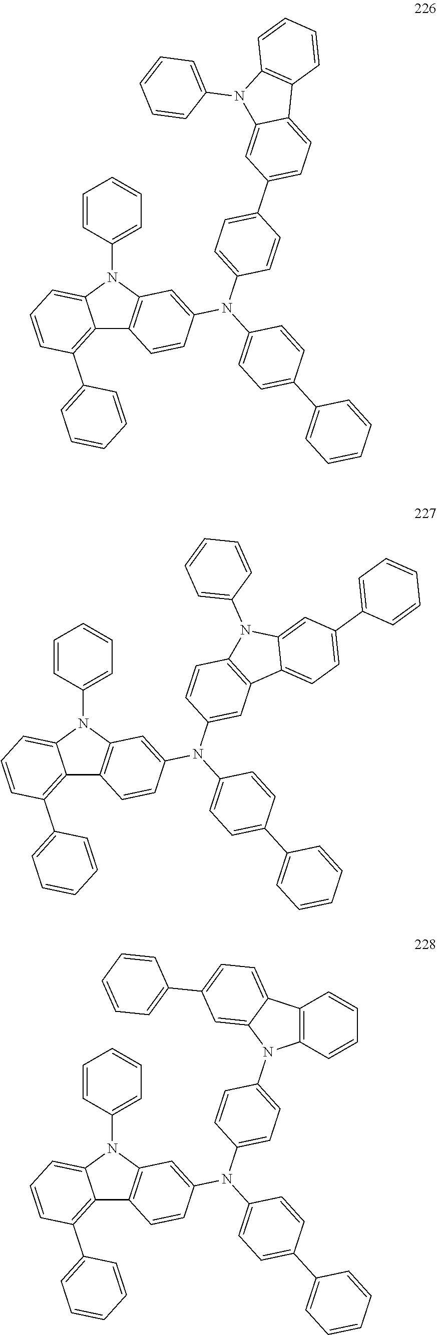 [SMILES: c1ccc(-c2ccc(N(c3ccc(-c4ccc5c6ccccc6n(-c6ccccc6)c5c4)cc3)c3ccc4c5c(-c6ccccc6)cccc5n(-c5ccccc5)c4c3)cc2)cc1 ; c1ccc(-c2ccc(N(c3ccc4c(c3)c3ccc(-c5ccccc5)cc3n4-c3ccccc3)c3ccc4c5c(-c6ccccc6)cccc5n(-c5ccccc5)c4c3)cc2)cc1 ; c1ccc(-c2ccc(N(c3ccc(-n4c5ccccc5c5ccc(-c6ccccc6)cc54)cc3)c3ccc4c5c(-c6ccccc6)cccc5n(-c5ccccc5)c4c3)cc2)cc1]

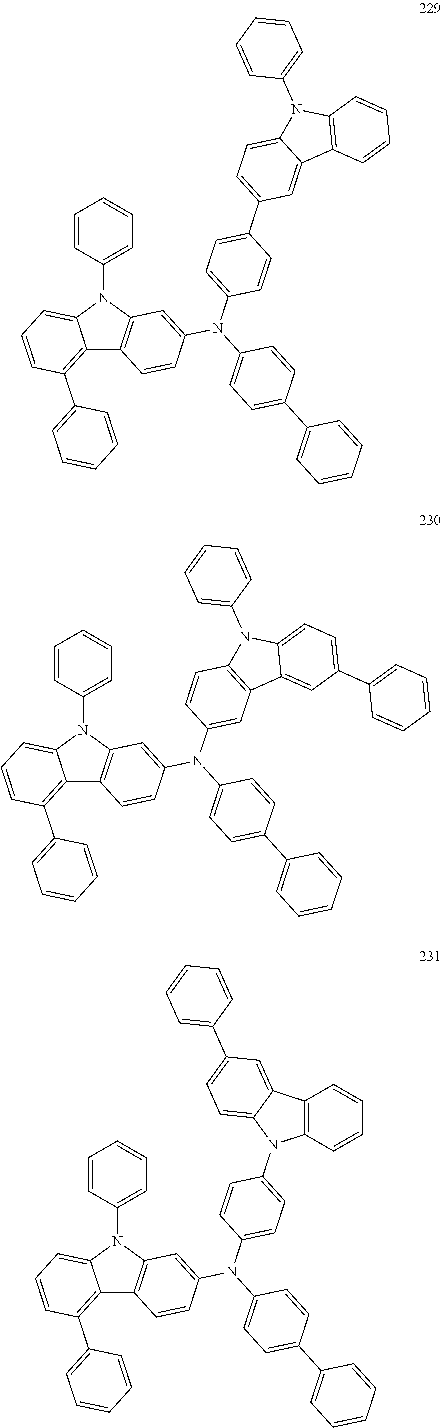 [SMILES: c1ccc(-c2ccc(N(c3ccc(-c4ccc5c(c4)c4ccccc4n5-c4ccccc4)cc3)c3ccc4c5c(-c6ccccc6)cccc5n(-c5ccccc5)c4c3)cc2)cc1 ; c1ccc(-c2ccc(N(c3ccc4c(c3)c3cc(-c5ccccc5)ccc3n4-c3ccccc3)c3ccc4c5c(-c6ccccc6)cccc5n(-c5ccccc5)c4c3)cc2)cc1 ; c1ccc(-c2ccc(N(c3ccc(-n4c5ccccc5c5cc(-c6ccccc6)ccc54)cc3)c3ccc4c5c(-c6ccccc6)cccc5n(-c5ccccc5)c4c3)cc2)cc1]

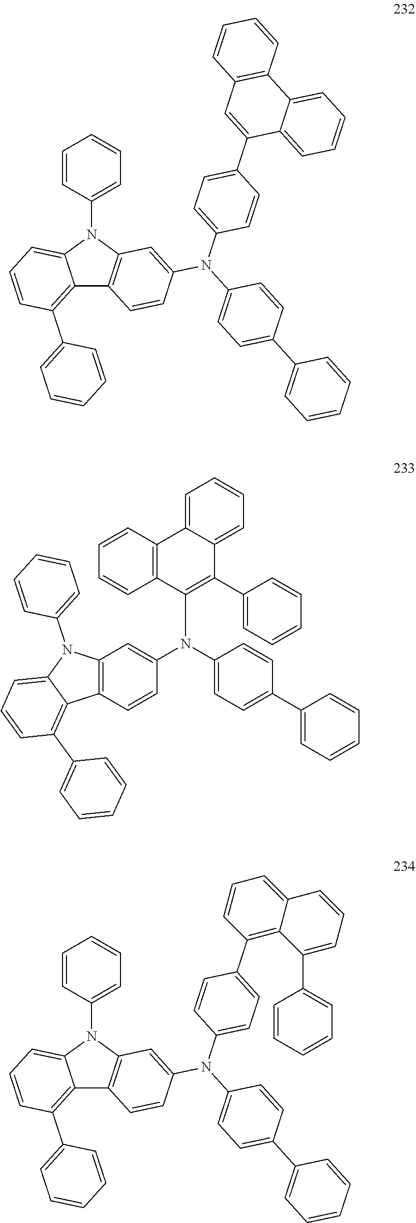 [SMILES: c1ccc(-c2ccc(N(c3ccc(-c4cc5ccccc5c5ccccc45)cc3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3c(-c4ccccc4)c4ccccc4c4ccccc34)cc2)cc1 ; c1ccc(-c2ccc(N(c3ccc(-c4cccc5cccc(-c6ccccc6)c45)cc3)c3ccc4c5c(-c6ccccc6)cccc5n(-c5ccccc5)c4c3)cc2)cc1]

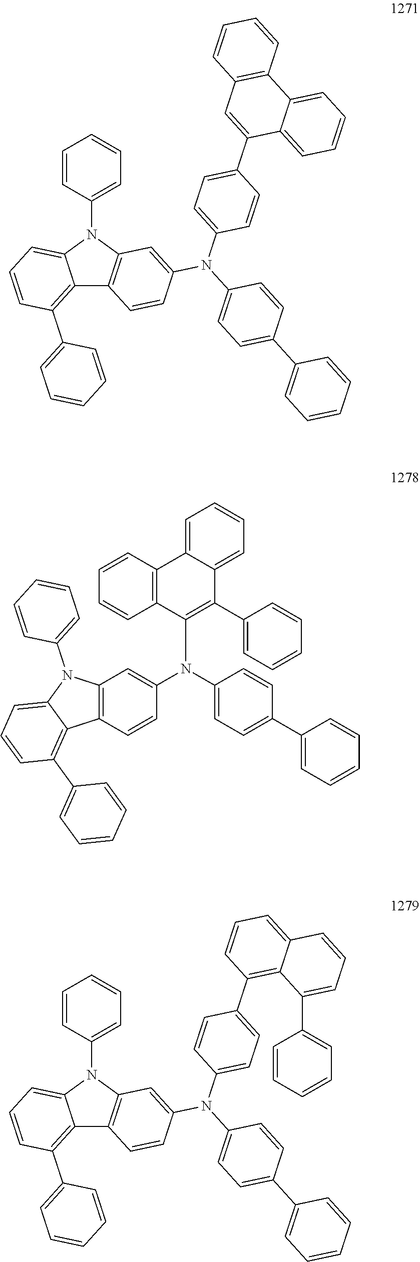 [SMILES: c1ccc(-c2ccc(N(c3ccc(-c4cc5ccccc5c5ccccc45)cc3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3c(-c4ccccc4)c4ccccc4c4ccccc34)cc2)cc1 ; c1ccc(-c2ccc(N(c3ccc(-c4cccc5cccc(-c6ccccc6)c45)cc3)c3ccc4c5c(-c6ccccc6)cccc5n(-c5ccccc5)c4c3)cc2)cc1]

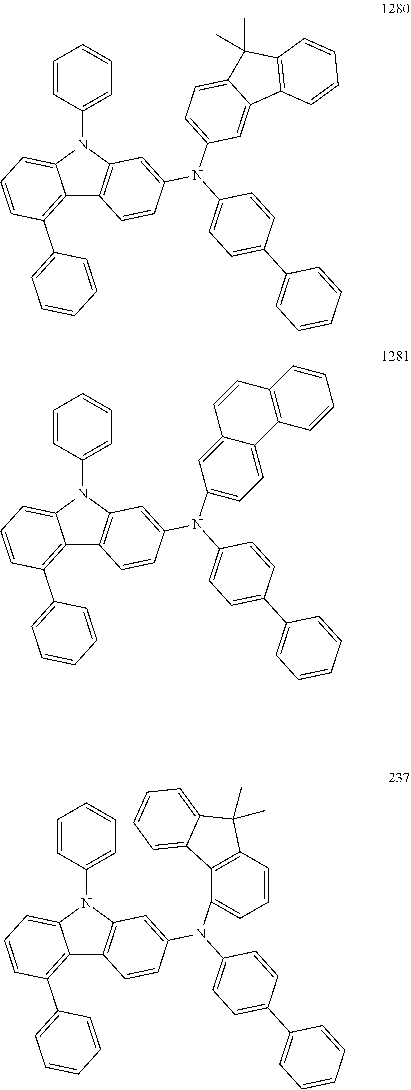

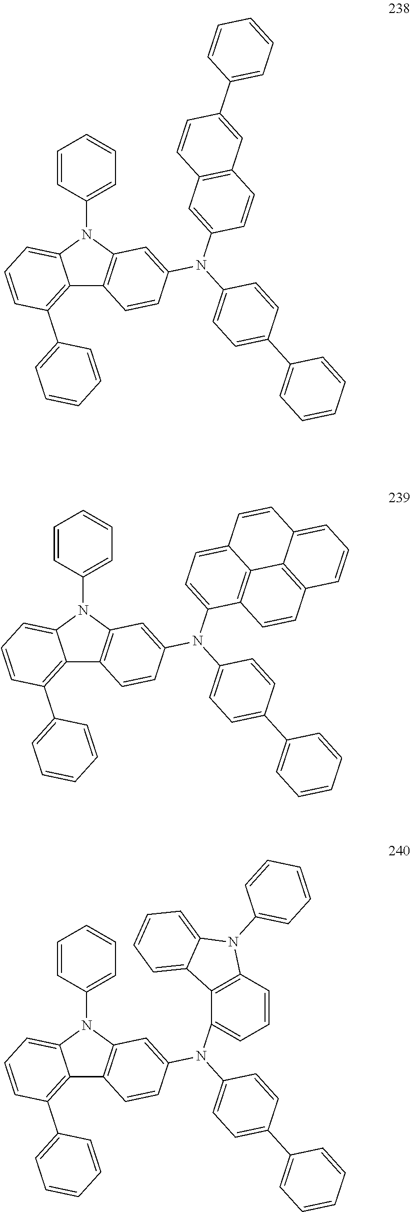 [SMILES: c1ccc(-c2ccc(N(c3ccc4cc(-c5ccccc5)ccc4c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3ccc4ccc5cccc6ccc3c4c56)cc2)cc1 ; c1ccc(-c2ccc(N(c3ccc4c5c(-c6ccccc6)cccc5n(-c5ccccc5)c4c3)c3cccc4c3c3ccccc3n4-c3ccccc3)cc2)cc1]

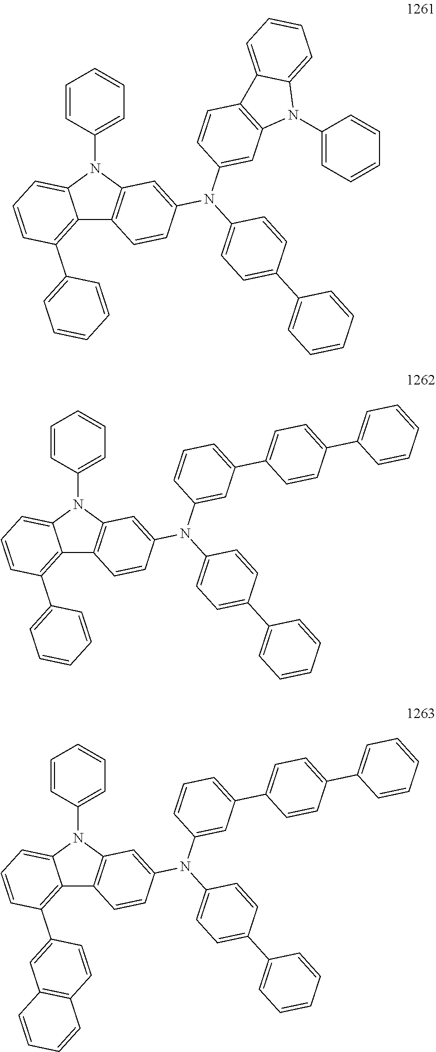 [SMILES: c1ccc(-c2ccc(N(c3ccc4c5ccccc5n(-c5ccccc5)c4c3)c3ccc4c5c(-c6ccccc6)cccc5n(-c5ccccc5)c4c3)cc2)cc1 ; c1ccc(-c2ccc(-c3cccc(N(c4ccc(-c5ccccc5)cc4)c4ccc5c6c(-c7ccccc7)cccc6n(-c6ccccc6)c5c4)c3)cc2)cc1 ; c1ccc(-c2ccc(-c3cccc(N(c4ccc(-c5ccccc5)cc4)c4ccc5c6c(-c7ccc8ccccc8c7)cccc6n(-c6ccccc6)c5c4)c3)cc2)cc1]

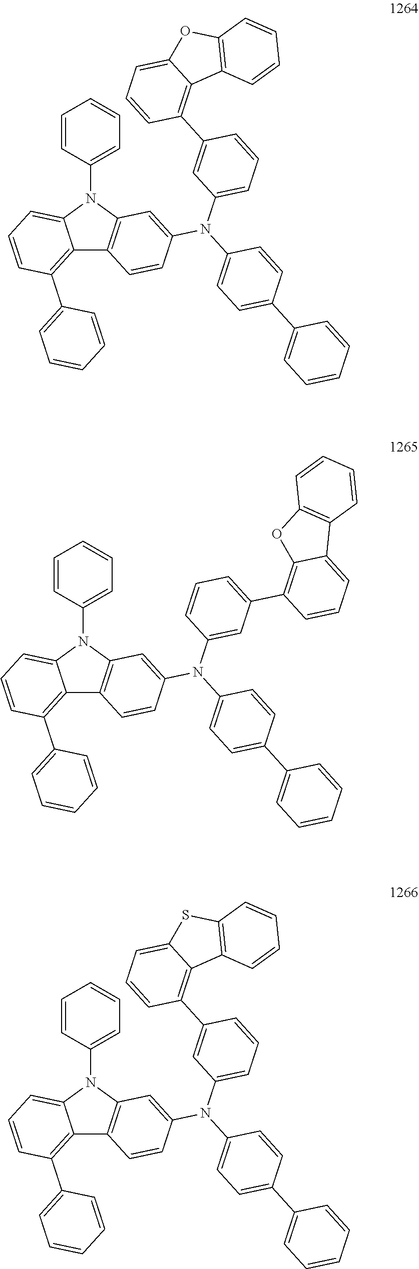 [SMILES: c1ccc(-c2ccc(N(c3cccc(-c4cccc5oc6ccccc6c45)c3)c3ccc4c5c(-c6ccccc6)cccc5n(-c5ccccc5)c4c3)cc2)cc1 ; c1ccc(-c2ccc(N(c3cccc(-c4cccc5c4oc4ccccc45)c3)c3ccc4c5c(-c6ccccc6)cccc5n(-c5ccccc5)c4c3)cc2)cc1 ; c1ccc(-c2ccc(N(c3cccc(-c4cccc5sc6ccccc6c45)c3)c3ccc4c5c(-c6ccccc6)cccc5n(-c5ccccc5)c4c3)cc2)cc1]

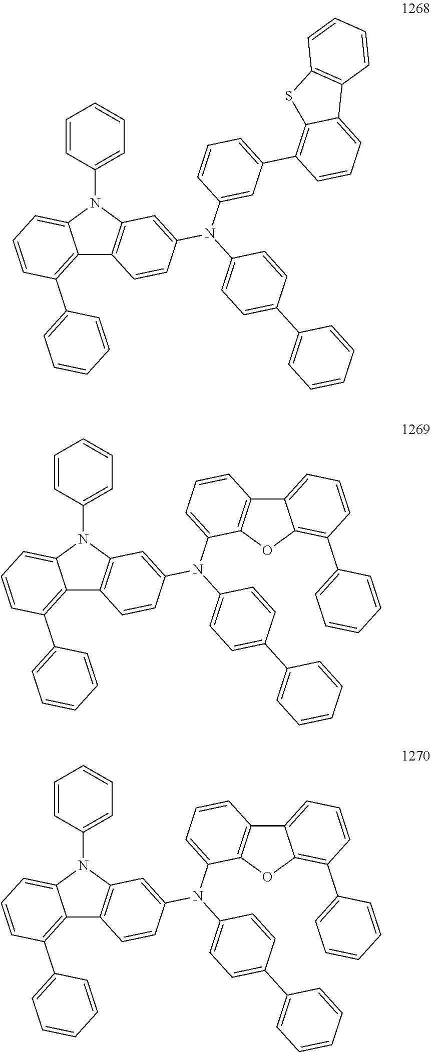 [SMILES: c1ccc(-c2ccc(N(c3cccc(-c4cccc5c4sc4ccccc45)c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3cccc4c3oc3c(-c5ccccc5)cccc34)cc2)cc1 ; c1ccc(-c2ccc(N(c3ccc4c5c(-c6ccccc6)cccc5n(-c5ccccc5)c4c3)c3cccc4c3oc3c(-c5ccccc5)cccc34)cc2)cc1]

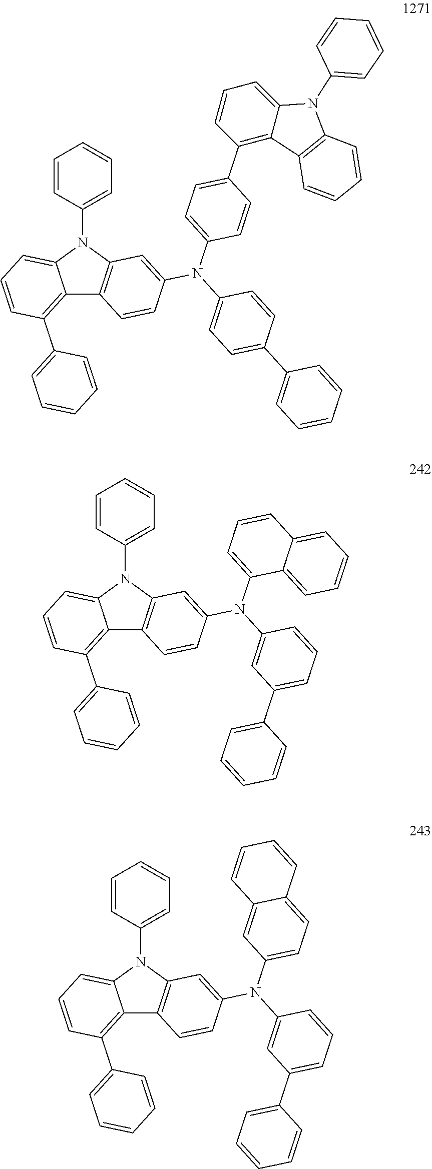 [SMILES: c1ccc(-c2ccc(N(c3ccc(-c4cccc5c4c4ccccc4n5-c4ccccc4)cc3)c3ccc4c5c(-c6ccccc6)cccc5n(-c5ccccc5)c4c3)cc2)cc1 ; c1ccc(-c2cccc(N(c3ccc4c5c(-c6ccccc6)cccc5n(-c5ccccc5)c4c3)c3cccc4ccccc34)c2)cc1 ; c1ccc(-c2cccc(N(c3ccc4ccccc4c3)c3ccc4c5c(-c6ccccc6)cccc5n(-c5ccccc5)c4c3)c2)cc1]

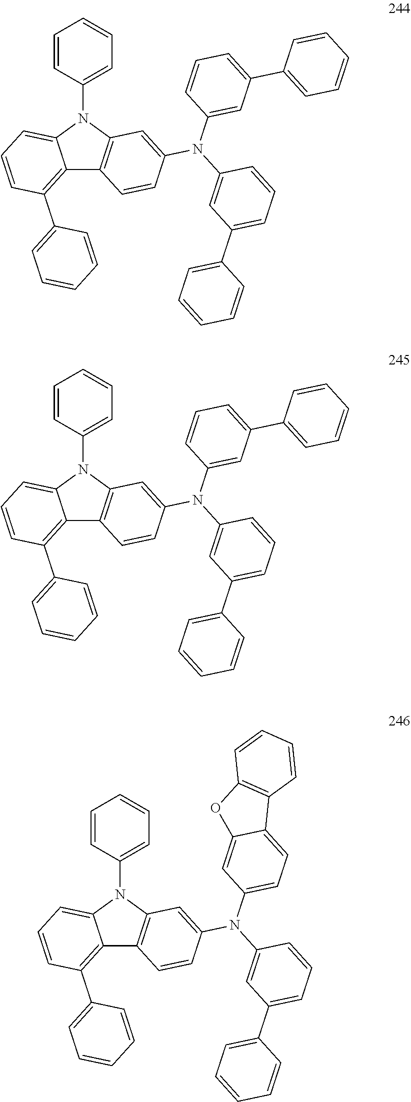 [SMILES: c1ccc(-c2cccc(N(c3cccc(-c4ccccc4)c3)c3ccc4c5c(-c6ccccc6)cccc5n(-c5ccccc5)c4c3)c2)cc1 ; c1ccc(-c2cccc(N(c3cccc(-c4ccccc4)c3)c3ccc4c5c(-c6ccccc6)cccc5n(-c5ccccc5)c4c3)c2)cc1 ; c1ccc(-c2cccc(N(c3ccc4c(c3)oc3ccccc34)c3ccc4c5c(-c6ccccc6)cccc5n(-c5ccccc5)c4c3)c2)cc1]

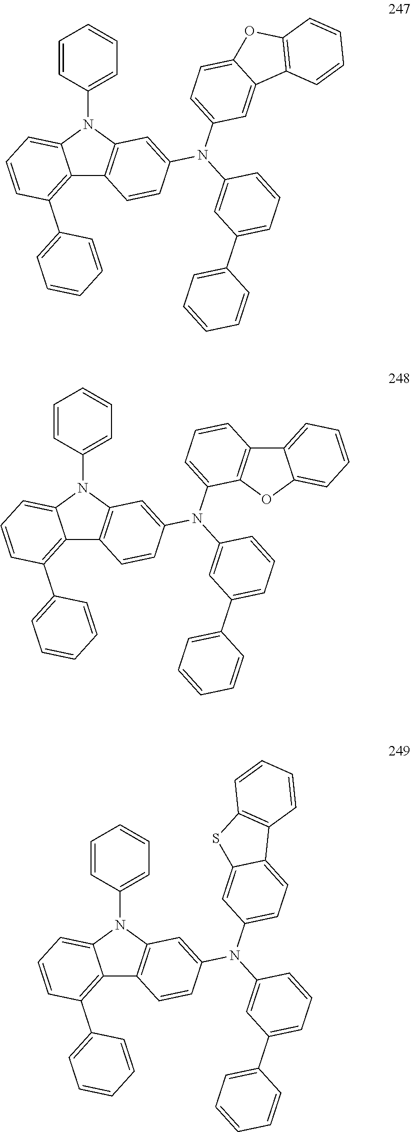 [SMILES: c1ccc(-c2cccc(N(c3ccc4oc5ccccc5c4c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3oc3ccccc34)c2)cc1 ; c1ccc(-c2cccc(N(c3ccc4c(c3)sc3ccccc34)c3ccc4c5c(-c6ccccc6)cccc5n(-c5ccccc5)c4c3)c2)cc1]

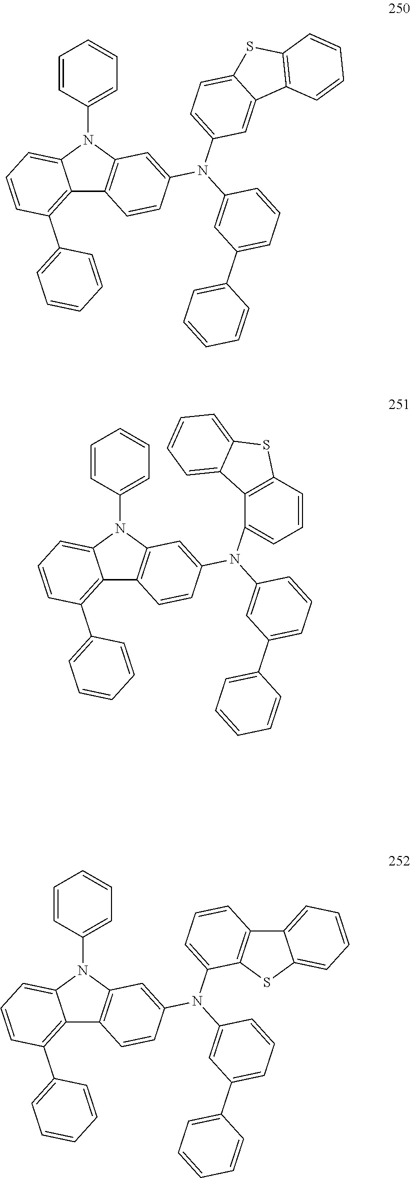 [SMILES: c1ccc(-c2cccc(N(c3ccc4sc5ccccc5c4c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4sc5ccccc5c34)c2)cc1 ; c1ccc(-c2cccc(N(c3ccc4c5c(-c6ccccc6)cccc5n(-c5ccccc5)c4c3)c3cccc4c3sc3ccccc34)c2)cc1]

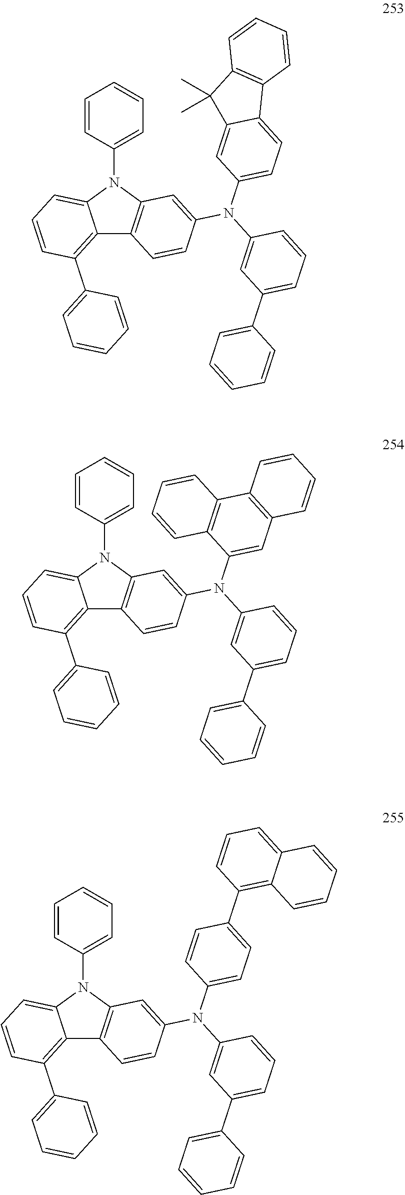 [SMILES: CC1(C)c2ccccc2-c2ccc(N(c3cccc(-c4ccccc4)c3)c3ccc4c5c(-c6ccccc6)cccc5n(-c5ccccc5)c4c3)cc21 ; c1ccc(-c2cccc(N(c3ccc4c5c(-c6ccccc6)cccc5n(-c5ccccc5)c4c3)c3cc4ccccc4c4ccccc34)c2)cc1 ; c1ccc(-c2cccc(N(c3ccc(-c4cccc5ccccc45)cc3)c3ccc4c5c(-c6ccccc6)cccc5n(-c5ccccc5)c4c3)c2)cc1]

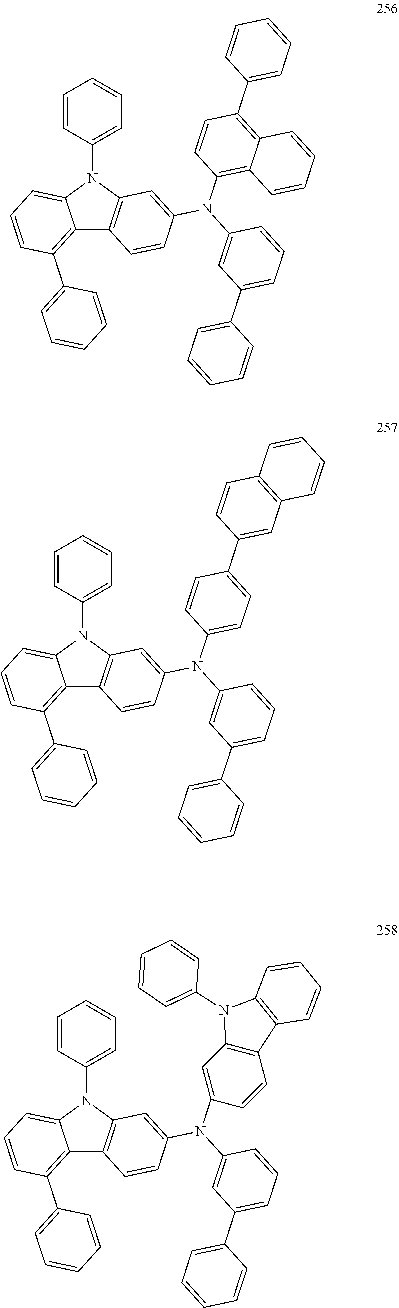 [SMILES: c1ccc(-c2cccc(N(c3ccc4c5c(-c6ccccc6)cccc5n(-c5ccccc5)c4c3)c3ccc(-c4ccccc4)c4ccccc34)c2)cc1 ; c1ccc(-c2cccc(N(c3ccc(-c4ccc5ccccc5c4)cc3)c3ccc4c5c(-c6ccccc6)cccc5n(-c5ccccc5)c4c3)c2)cc1 ; c1ccc(-c2cccc(N(c3ccc4c5ccccc5n(-c5ccccc5)c4c3)c3ccc4c5c(-c6ccccc6)cccc5n(-c5ccccc5)c4c3)c2)cc1]

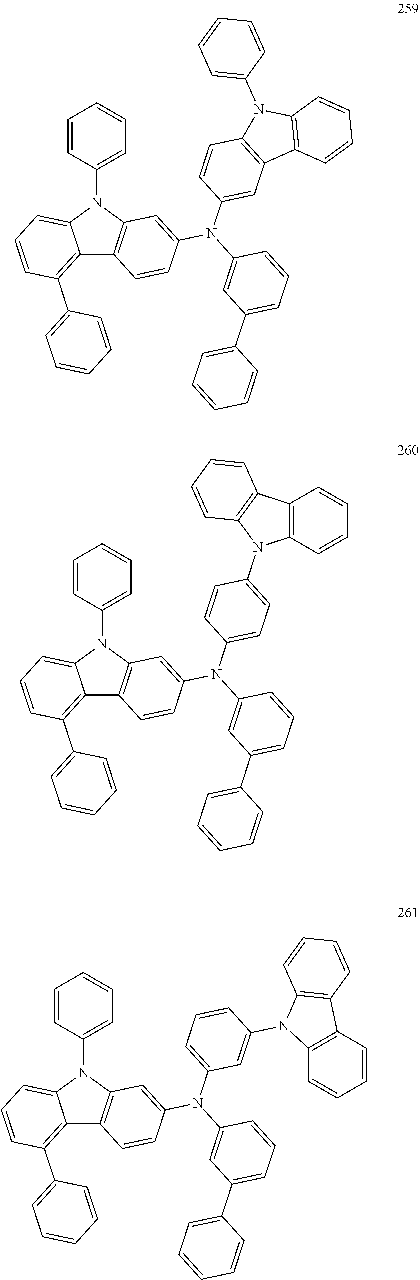 [SMILES: c1ccc(-c2cccc(N(c3ccc4c(c3)c3ccccc3n4-c3ccccc3)c3ccc4c5c(-c6ccccc6)cccc5n(-c5ccccc5)c4c3)c2)cc1 ; c1ccc(-c2cccc(N(c3ccc(-n4c5ccccc5c5ccccc54)cc3)c3ccc4c5c(-c6ccccc6)cccc5n(-c5ccccc5)c4c3)c2)cc1 ; c1ccc(-c2cccc(N(c3cccc(-n4c5ccccc5c5ccccc54)c3)c3ccc4c5c(-c6ccccc6)cccc5n(-c5ccccc5)c4c3)c2)cc1]

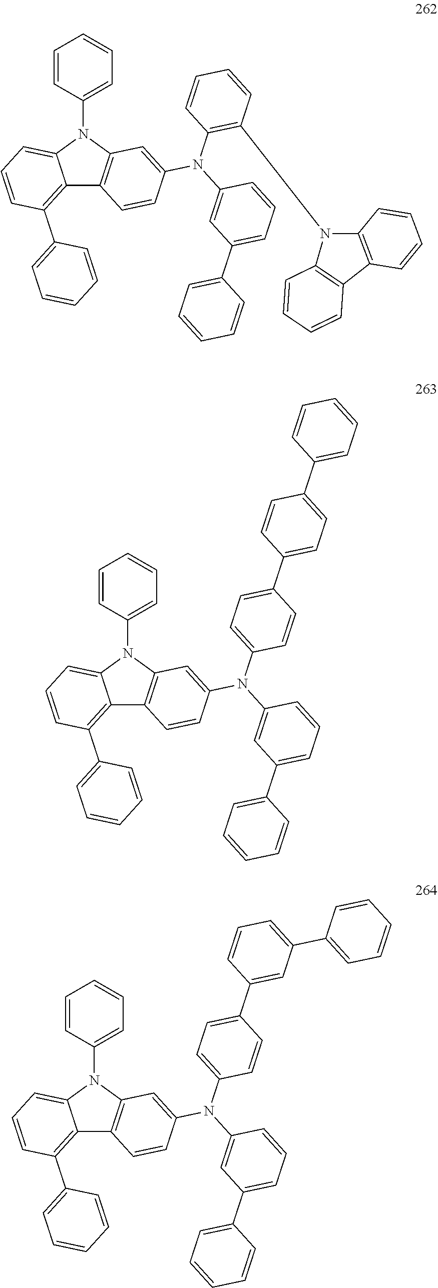 [SMILES: c1ccc(-c2cccc(N(c3ccc4c5c(-c6ccccc6)cccc5n(-c5ccccc5)c4c3)c3ccccc3-n3c4ccccc4c4ccccc43)c2)cc1 ; c1ccc(-c2ccc(-c3ccc(N(c4cccc(-c5ccccc5)c4)c4ccc5c6c(-c7ccccc7)cccc6n(-c6ccccc6)c5c4)cc3)cc2)cc1 ; c1ccc(-c2cccc(-c3ccc(N(c4cccc(-c5ccccc5)c4)c4ccc5c6c(-c7ccccc7)cccc6n(-c6ccccc6)c5c4)cc3)c2)cc1]

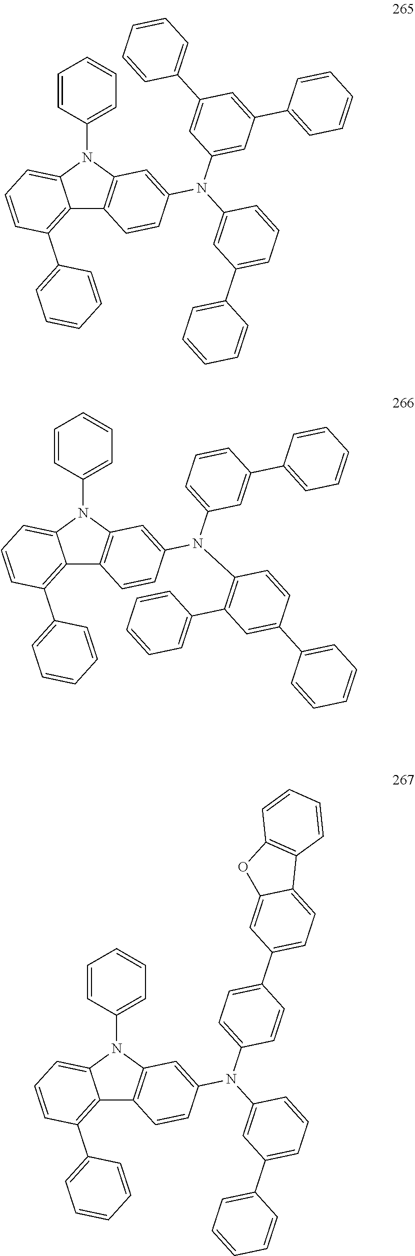 [SMILES: c1ccc(-c2cccc(N(c3cc(-c4ccccc4)cc(-c4ccccc4)c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3ccc(-c4ccccc4)cc3-c3ccccc3)c2)cc1 ; c1ccc(-c2cccc(N(c3ccc(-c4ccc5c(c4)oc4ccccc45)cc3)c3ccc4c5c(-c6ccccc6)cccc5n(-c5ccccc5)c4c3)c2)cc1]

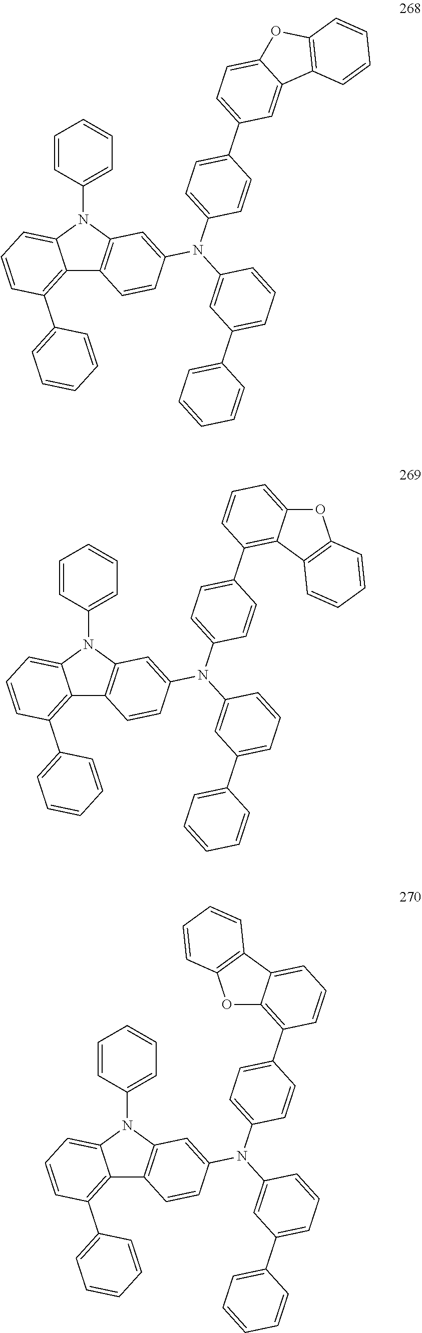 [SMILES: c1ccc(-c2cccc(N(c3ccc(-c4ccc5oc6ccccc6c5c4)cc3)c3ccc4c5c(-c6ccccc6)cccc5n(-c5ccccc5)c4c3)c2)cc1 ; c1ccc(-c2cccc(N(c3ccc(-c4cccc5oc6ccccc6c45)cc3)c3ccc4c5c(-c6ccccc6)cccc5n(-c5ccccc5)c4c3)c2)cc1 ; c1ccc(-c2cccc(N(c3ccc(-c4cccc5c4oc4ccccc45)cc3)c3ccc4c5c(-c6ccccc6)cccc5n(-c5ccccc5)c4c3)c2)cc1]

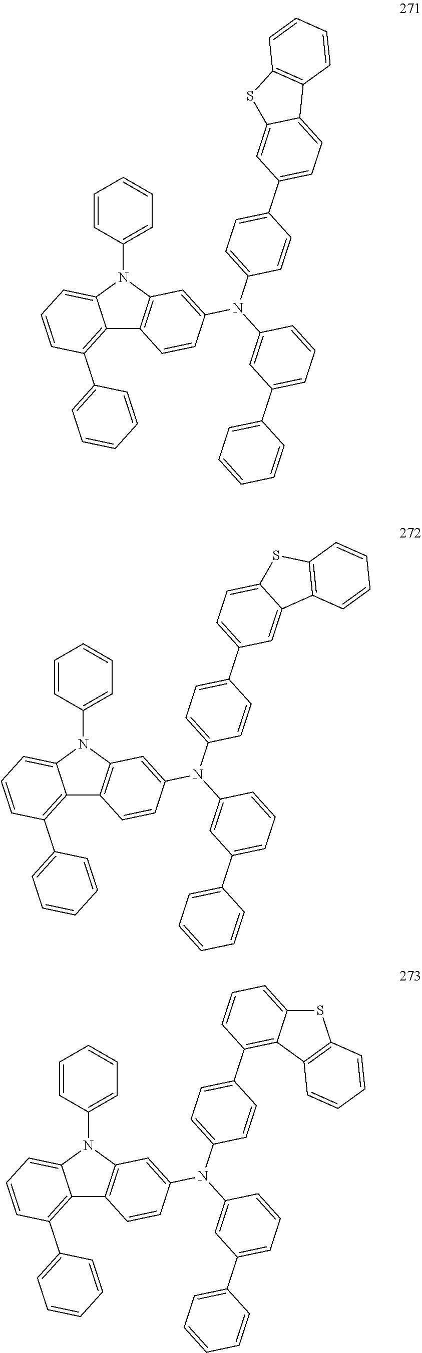 [SMILES: c1ccc(-c2cccc(N(c3ccc(-c4ccc5c(c4)sc4ccccc45)cc3)c3ccc4c5c(-c6ccccc6)cccc5n(-c5ccccc5)c4c3)c2)cc1 ; c1ccc(-c2cccc(N(c3ccc(-c4ccc5sc6ccccc6c5c4)cc3)c3ccc4c5c(-c6ccccc6)cccc5n(-c5ccccc5)c4c3)c2)cc1 ; c1ccc(-c2cccc(N(c3ccc(-c4cccc5sc6ccccc6c45)cc3)c3ccc4c5c(-c6ccccc6)cccc5n(-c5ccccc5)c4c3)c2)cc1]

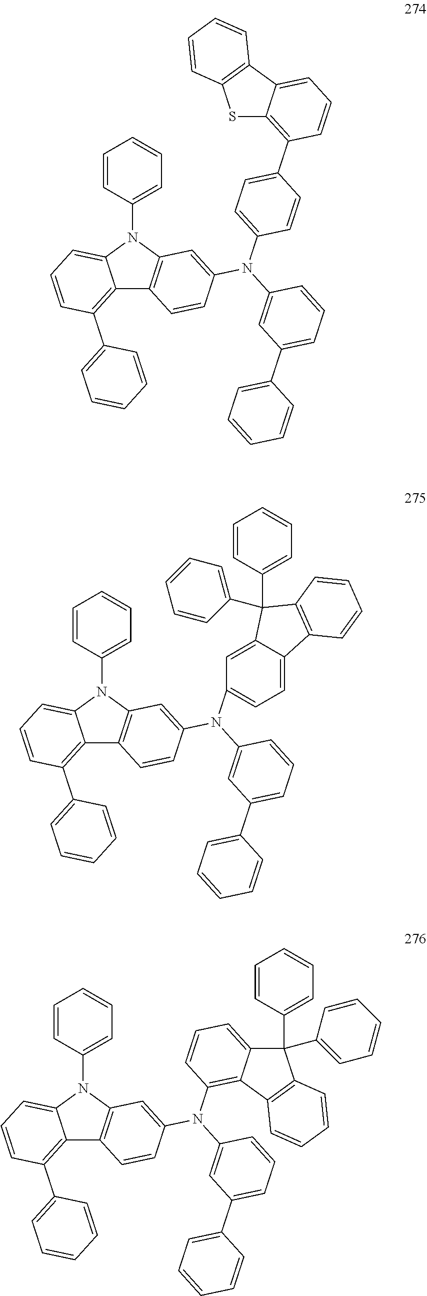 [SMILES: c1ccc(-c2cccc(N(c3ccc(-c4cccc5c4sc4ccccc45)cc3)c3ccc4c5c(-c6ccccc6)cccc5n(-c5ccccc5)c4c3)c2)cc1 ; c1ccc(-c2cccc(N(c3ccc4c(c3)C(c3ccccc3)(c3ccccc3)c3ccccc3-4)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3-c3ccccc3C4(c3ccccc3)c3ccccc3)c2)cc1]

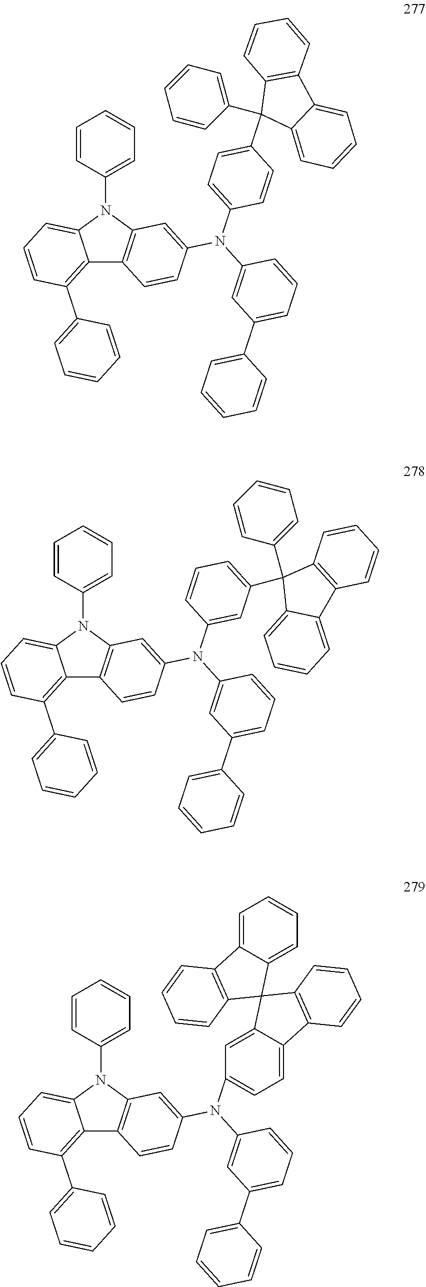 [SMILES: c1ccc(-c2cccc(N(c3ccc(C4(c5ccccc5)c5ccccc5-c5ccccc54)cc3)c3ccc4c5c(-c6ccccc6)cccc5n(-c5ccccc5)c4c3)c2)cc1 ; c1ccc(-c2cccc(N(c3cccc(C4(c5ccccc5)c5ccccc5-c5ccccc54)c3)c3ccc4c5c(-c6ccccc6)cccc5n(-c5ccccc5)c4c3)c2)cc1 ; c1ccc(-c2cccc(N(c3ccc4c(c3)C3(c5ccccc5-c5ccccc53)c3ccccc3-4)c3ccc4c5c(-c6ccccc6)cccc5n(-c5ccccc5)c4c3)c2)cc1]

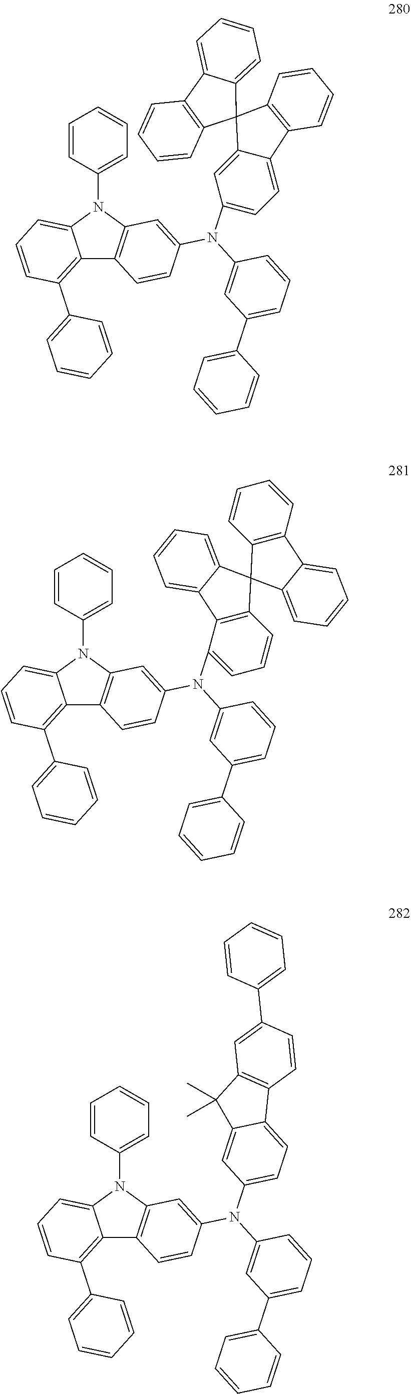 [SMILES: c1ccc(-c2cccc(N(c3ccc4c(c3)C3(c5ccccc5-c5ccccc53)c3ccccc3-4)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3-c3ccccc3C43c4ccccc4-c4ccccc43)c2)cc1 ; CC1(C)c2cc(-c3ccccc3)ccc2-c2ccc(N(c3cccc(-c4ccccc4)c3)c3ccc4c5c(-c6ccccc6)cccc5n(-c5ccccc5)c4c3)cc21]

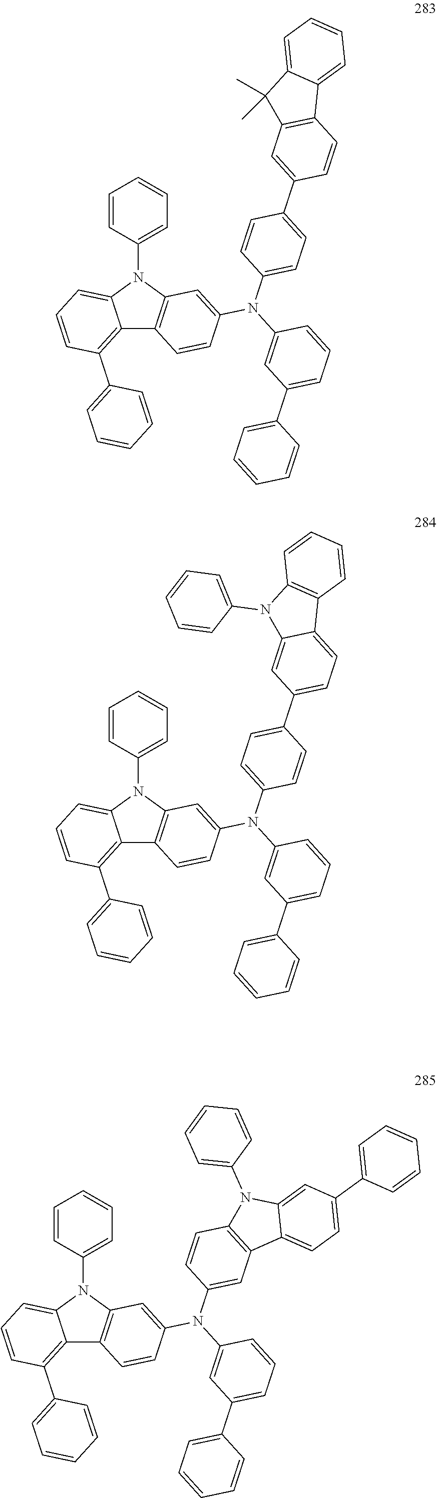 [SMILES: CC1(C)c2ccccc2-c2ccc(-c3ccc(N(c4cccc(-c5ccccc5)c4)c4ccc5c6c(-c7ccccc7)cccc6n(-c6ccccc6)c5c4)cc3)cc21 ; c1ccc(-c2cccc(N(c3ccc(-c4ccc5c6ccccc6n(-c6ccccc6)c5c4)cc3)c3ccc4c5c(-c6ccccc6)cccc5n(-c5ccccc5)c4c3)c2)cc1 ; c1ccc(-c2cccc(N(c3ccc4c(c3)c3ccc(-c5ccccc5)cc3n4-c3ccccc3)c3ccc4c5c(-c6ccccc6)cccc5n(-c5ccccc5)c4c3)c2)cc1]

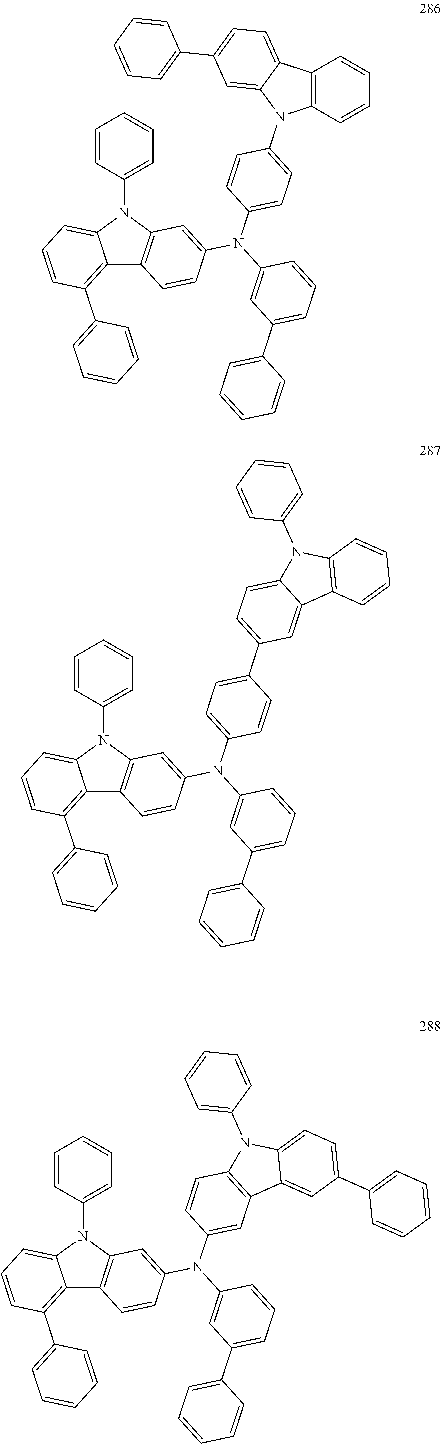

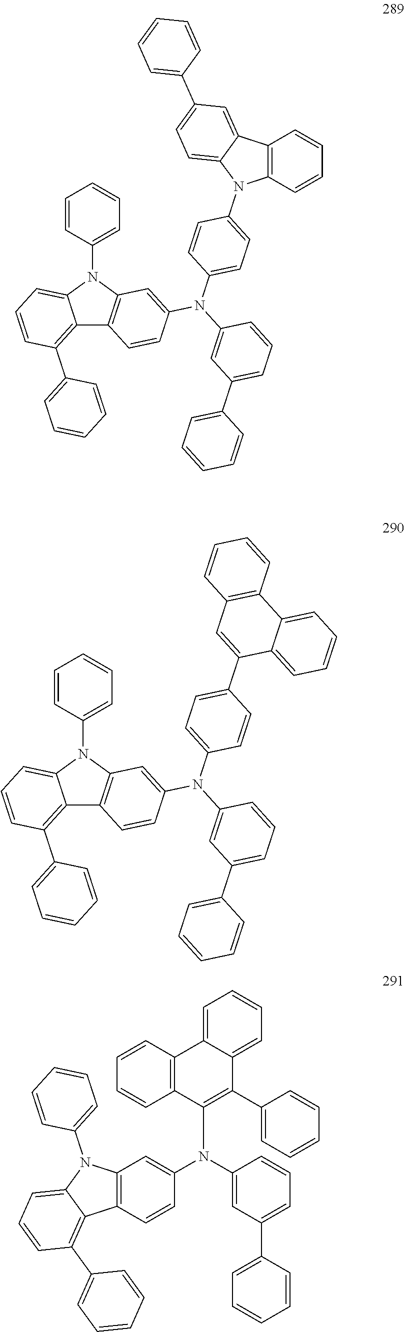

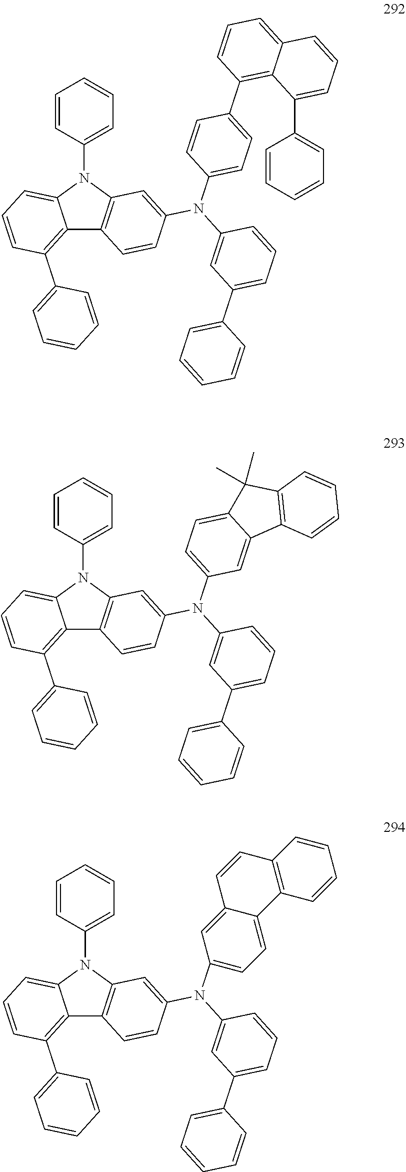 [SMILES: c1ccc(-c2cccc(N(c3ccc(-c4cccc5cccc(-c6ccccc6)c45)cc3)c3ccc4c5c(-c6ccccc6)cccc5n(-c5ccccc5)c4c3)c2)cc1 ; CC1(C)c2ccccc2-c2cc(N(c3cccc(-c4ccccc4)c3)c3ccc4c5c(-c6ccccc6)cccc5n(-c5ccccc5)c4c3)ccc21 ; c1ccc(-c2cccc(N(c3ccc4c(ccc5ccccc54)c3)c3ccc4c5c(-c6ccccc6)cccc5n(-c5ccccc5)c4c3)c2)cc1]

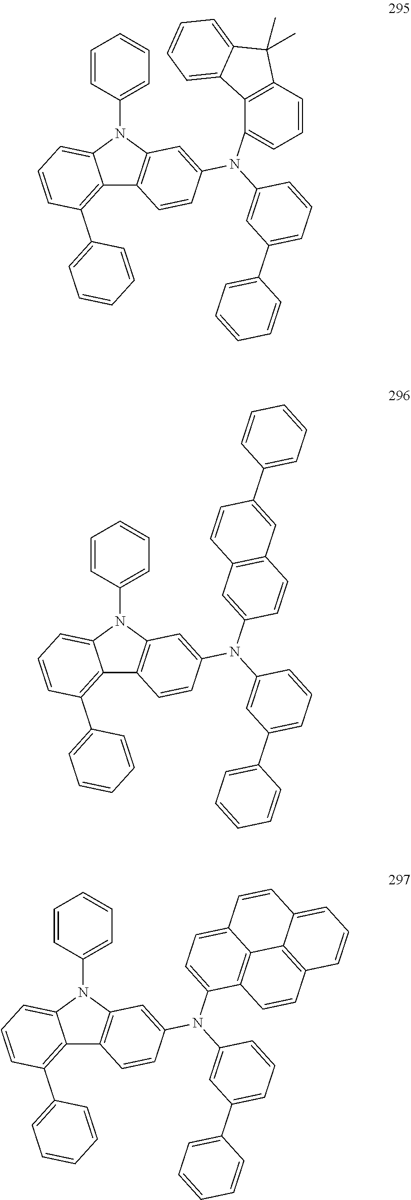 [SMILES: CC1(C)c2ccccc2-c2c(N(c3cccc(-c4ccccc4)c3)c3ccc4c5c(-c6ccccc6)cccc5n(-c5ccccc5)c4c3)cccc21 ; c1ccc(-c2cccc(N(c3ccc4cc(-c5ccccc5)ccc4c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3ccc4ccc5cccc6ccc3c4c56)c2)cc1]

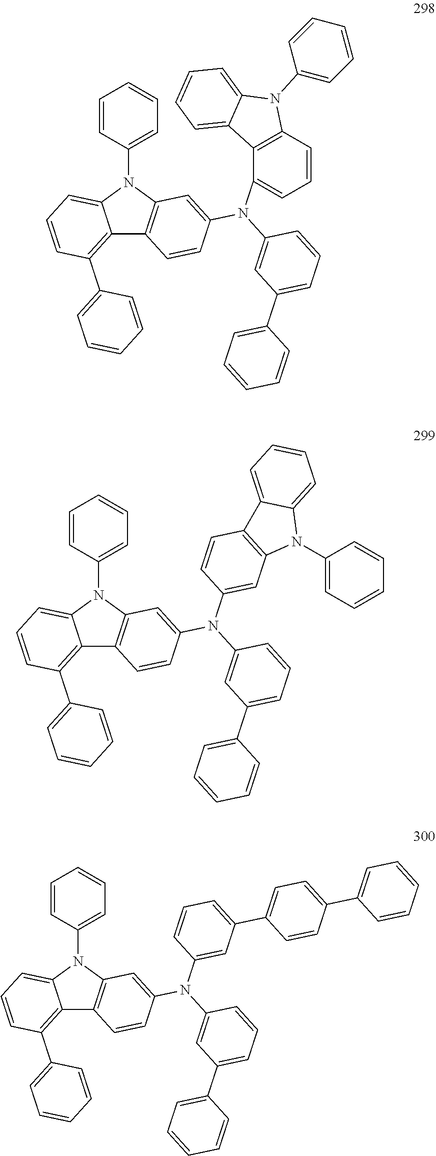 [SMILES: c1ccc(-c2cccc(N(c3ccc4c5c(-c6ccccc6)cccc5n(-c5ccccc5)c4c3)c3cccc4c3c3ccccc3n4-c3ccccc3)c2)cc1 ; c1ccc(-c2cccc(N(c3ccc4c5ccccc5n(-c5ccccc5)c4c3)c3ccc4c5c(-c6ccccc6)cccc5n(-c5ccccc5)c4c3)c2)cc1 ; c1ccc(-c2ccc(-c3cccc(N(c4cccc(-c5ccccc5)c4)c4ccc5c6c(-c7ccccc7)cccc6n(-c6ccccc6)c5c4)c3)cc2)cc1]

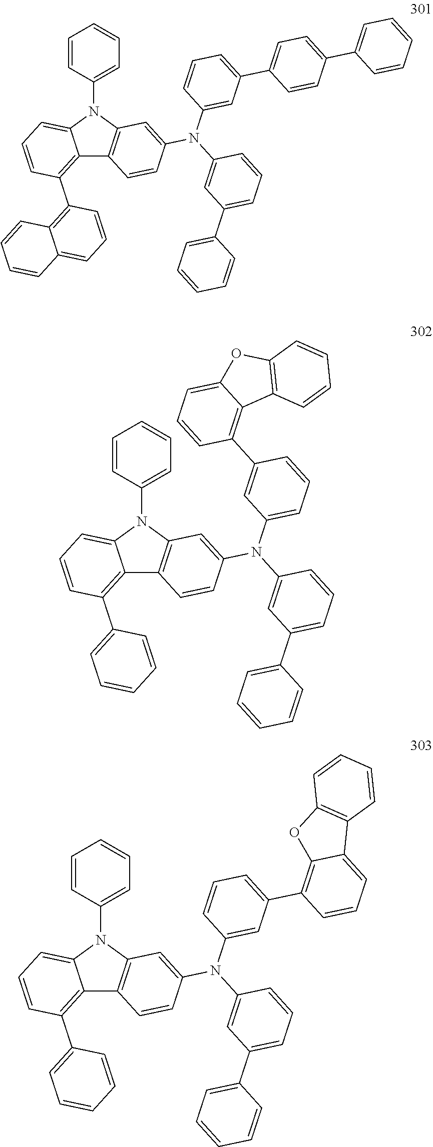 [SMILES: c1ccc(-c2ccc(-c3cccc(N(c4cccc(-c5ccccc5)c4)c4ccc5c6c(-c7cccc8ccccc78)cccc6n(-c6ccccc6)c5c4)c3)cc2)cc1 ; c1ccc(-c2cccc(N(c3cccc(-c4cccc5oc6ccccc6c45)c3)c3ccc4c5c(-c6ccccc6)cccc5n(-c5ccccc5)c4c3)c2)cc1 ; c1ccc(-c2cccc(N(c3cccc(-c4cccc5c4oc4ccccc45)c3)c3ccc4c5c(-c6ccccc6)cccc5n(-c5ccccc5)c4c3)c2)cc1]

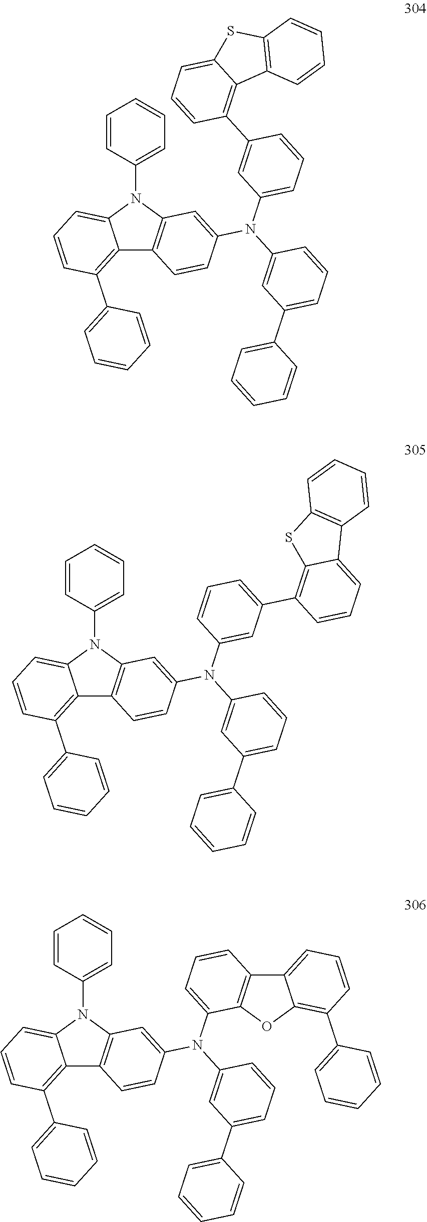 [SMILES: c1ccc(-c2cccc(N(c3cccc(-c4cccc5sc6ccccc6c45)c3)c3ccc4c5c(-c6ccccc6)cccc5n(-c5ccccc5)c4c3)c2)cc1 ; c1ccc(-c2cccc(N(c3cccc(-c4cccc5c4sc4ccccc45)c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3oc3c(-c5ccccc5)cccc34)c2)cc1]

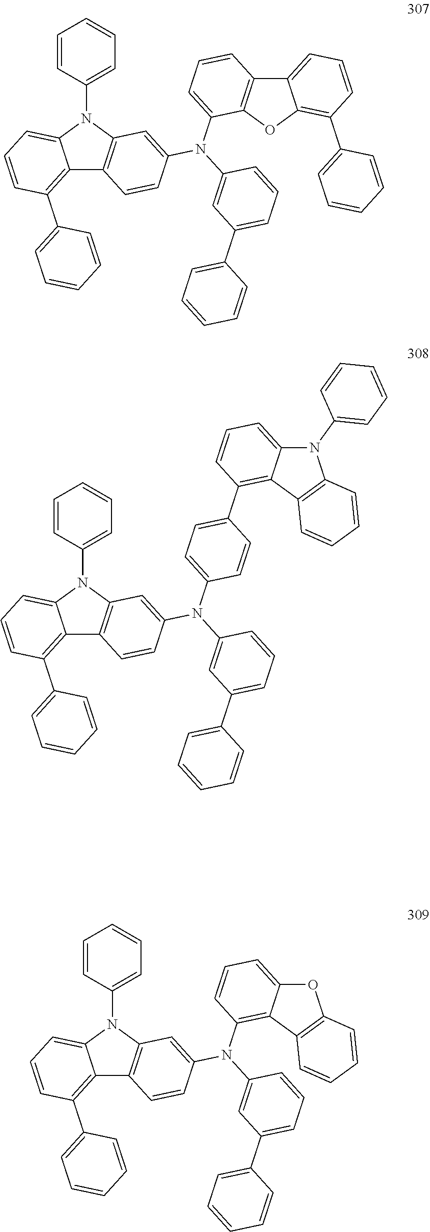 [SMILES: c1ccc(-c2cccc(N(c3ccc4c5c(-c6ccccc6)cccc5n(-c5ccccc5)c4c3)c3cccc4c3oc3c(-c5ccccc5)cccc34)c2)cc1 ; c1ccc(-c2cccc(N(c3ccc(-c4cccc5c4c4ccccc4n5-c4ccccc4)cc3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4oc5ccccc5c34)c2)cc1]

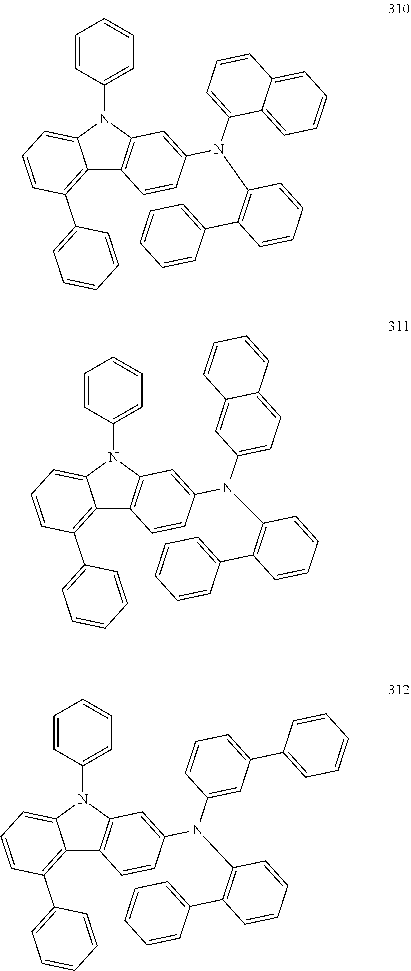 [SMILES: c1ccc(-c2ccccc2N(c2ccc3c4c(-c5ccccc5)cccc4n(-c4ccccc4)c3c2)c2cccc3ccccc23)cc1 ; c1ccc(-c2ccccc2N(c2ccc3ccccc3c2)c2ccc3c4c(-c5ccccc5)cccc4n(-c4ccccc4)c3c2)cc1 ; c1ccc(-c2cccc(N(c3ccc4c5c(-c6ccccc6)cccc5n(-c5ccccc5)c4c3)c3ccccc3-c3ccccc3)c2)cc1]

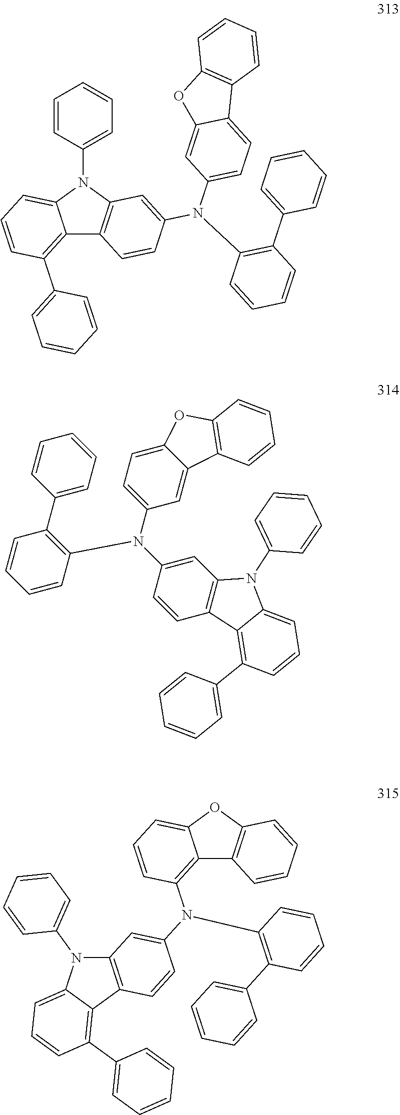 [SMILES: c1ccc(-c2ccccc2N(c2ccc3c(c2)oc2ccccc23)c2ccc3c4c(-c5ccccc5)cccc4n(-c4ccccc4)c3c2)cc1 ; c1ccc(-c2ccccc2N(c2ccc3oc4ccccc4c3c2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2cccc3oc4ccccc4c23)cc1]

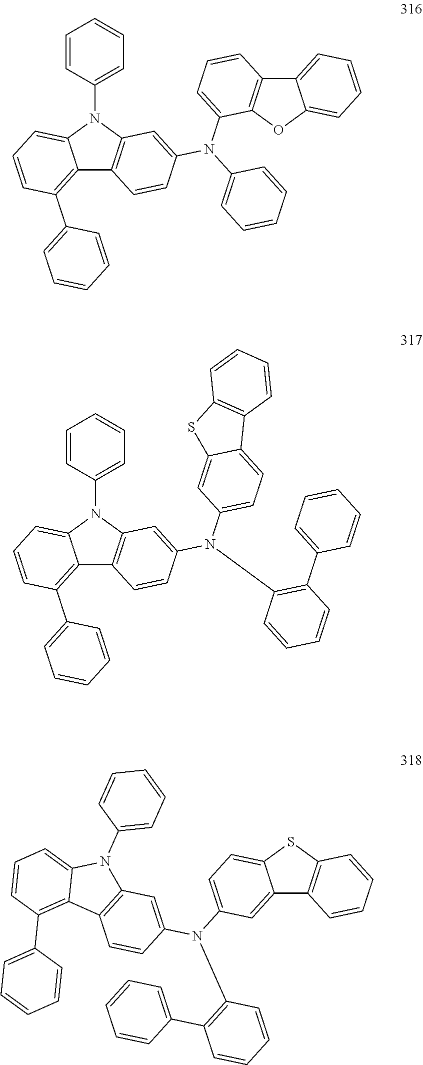 [SMILES: c1ccc(-c2cccc3c2c2ccc(N(c4ccccc4)c4cccc5c4oc4ccccc45)cc2n3-c2ccccc2)cc1 ; c1ccc(-c2ccccc2N(c2ccc3c(c2)sc2ccccc23)c2ccc3c4c(-c5ccccc5)cccc4n(-c4ccccc4)c3c2)cc1 ; c1ccc(-c2ccccc2N(c2ccc3sc4ccccc4c3c2)c2ccc3c4c(-c5ccccc5)cccc4n(-c4ccccc4)c3c2)cc1]

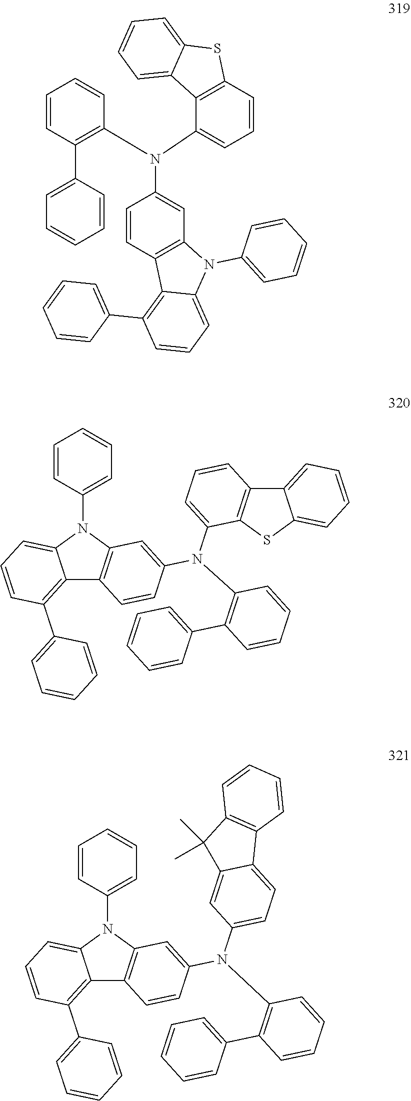 [SMILES: c1ccc(-c2ccccc2N(c2ccc3c4c(-c5ccccc5)cccc4n(-c4ccccc4)c3c2)c2cccc3sc4ccccc4c23)cc1 ; c1ccc(-c2ccccc2N(c2ccc3c4c(-c5ccccc5)cccc4n(-c4ccccc4)c3c2)c2cccc3c2sc2ccccc23)cc1 ; CC1(C)c2ccccc2-c2ccc(N(c3ccc4c5c(-c6ccccc6)cccc5n(-c5ccccc5)c4c3)c3ccccc3-c3ccccc3)cc21]

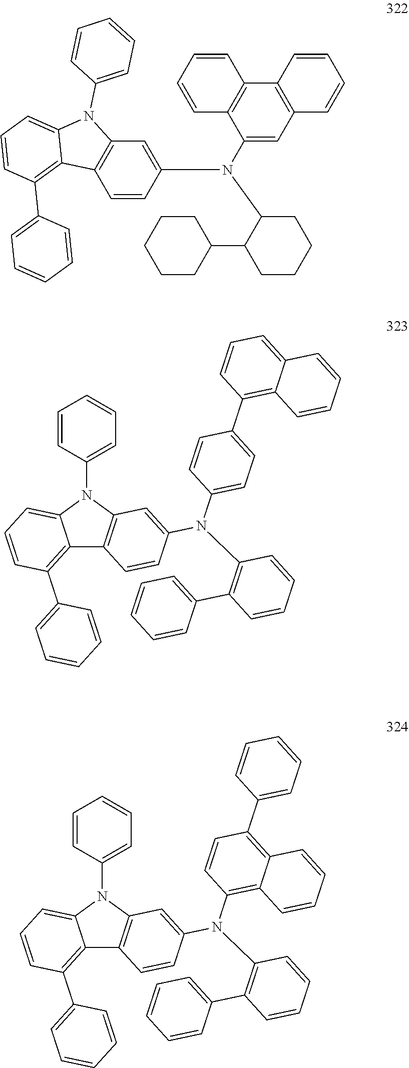 [SMILES: c1ccc(-c2cccc3c2c2ccc(N(c4cc5ccccc5c5ccccc45)C4CCCCC4C4CCCCC4)cc2n3-c2ccccc2)cc1 ; c1ccc(-c2ccccc2N(c2ccc(-c3cccc4ccccc34)cc2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2ccc(-c3ccccc3)c3ccccc23)cc1]

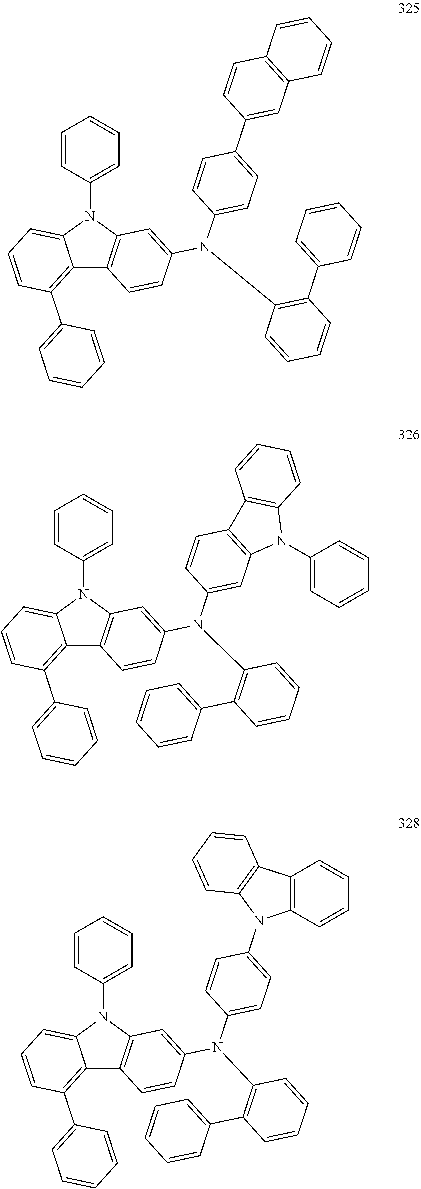 [SMILES: c1ccc(-c2ccccc2N(c2ccc(-c3ccc4ccccc4c3)cc2)c2ccc3c4c(-c5ccccc5)cccc4n(-c4ccccc4)c3c2)cc1 ; c1ccc(-c2ccccc2N(c2ccc3c4ccccc4n(-c4ccccc4)c3c2)c2ccc3c4c(-c5ccccc5)cccc4n(-c4ccccc4)c3c2)cc1 ; c1ccc(-c2ccccc2N(c2ccc(-n3c4ccccc4c4ccccc43)cc2)c2ccc3c4c(-c5ccccc5)cccc4n(-c4ccccc4)c3c2)cc1]

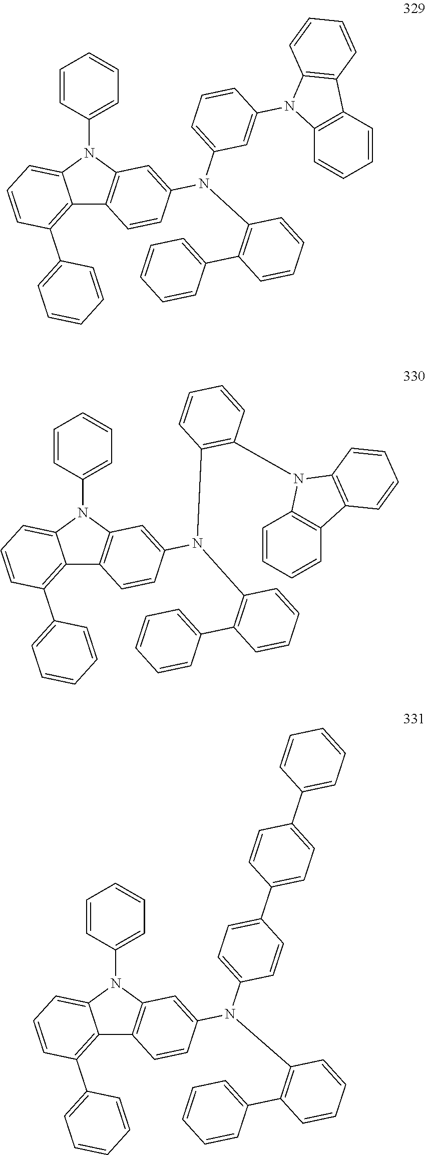 [SMILES: c1ccc(-c2ccccc2N(c2cccc(-n3c4ccccc4c4ccccc43)c2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2ccccc2-n2c3ccccc3c3ccccc32)cc1 ; c1ccc(-c2ccc(-c3ccc(N(c4ccc5c6c(-c7ccccc7)cccc6n(-c6ccccc6)c5c4)c4ccccc4-c4ccccc4)cc3)cc2)cc1]

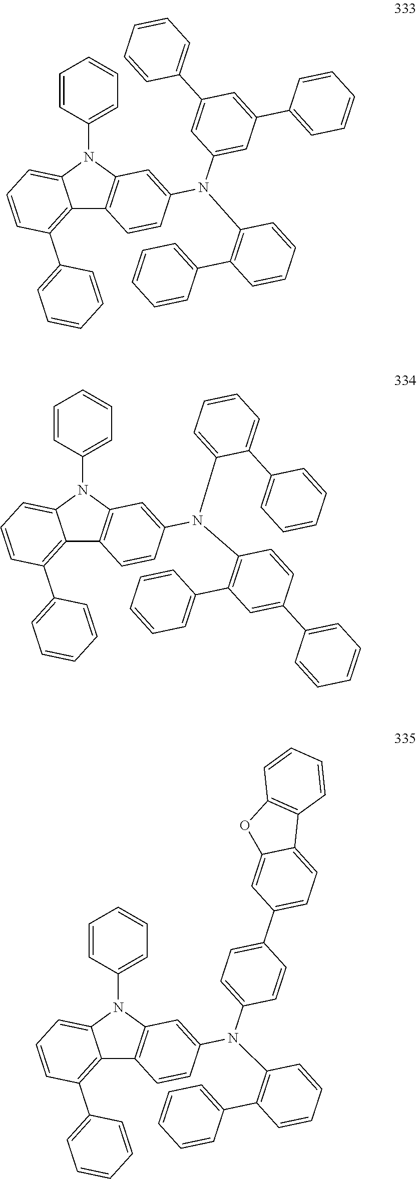 [SMILES: c1ccc(-c2cc(-c3ccccc3)cc(N(c3ccc4c5c(-c6ccccc6)cccc5n(-c5ccccc5)c4c3)c3ccccc3-c3ccccc3)c2)cc1 ; c1ccc(-c2ccc(N(c3ccc4c5c(-c6ccccc6)cccc5n(-c5ccccc5)c4c3)c3ccccc3-c3ccccc3)c(-c3ccccc3)c2)cc1 ; c1ccc(-c2ccccc2N(c2ccc(-c3ccc4c(c3)oc3ccccc34)cc2)c2ccc3c4c(-c5ccccc5)cccc4n(-c4ccccc4)c3c2)cc1]

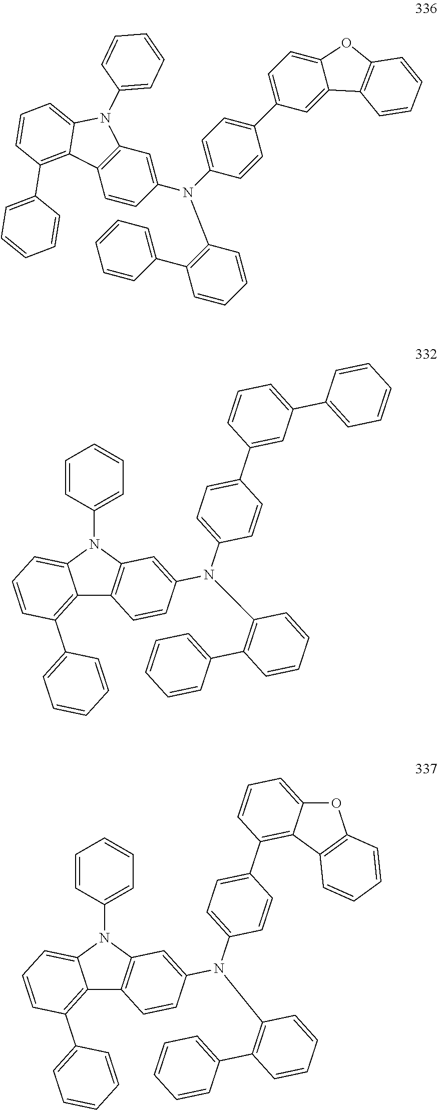 [SMILES: c1ccc(-c2ccccc2N(c2ccc(-c3ccc4oc5ccccc5c4c3)cc2)c2ccc3c4c(-c5ccccc5)cccc4n(-c4ccccc4)c3c2)cc1 ; c1ccc(-c2cccc(-c3ccc(N(c4ccc5c6c(-c7ccccc7)cccc6n(-c6ccccc6)c5c4)c4ccccc4-c4ccccc4)cc3)c2)cc1 ; c1ccc(-c2ccccc2N(c2ccc(-c3cccc4oc5ccccc5c34)cc2)c2ccc3c4c(-c5ccccc5)cccc4n(-c4ccccc4)c3c2)cc1]

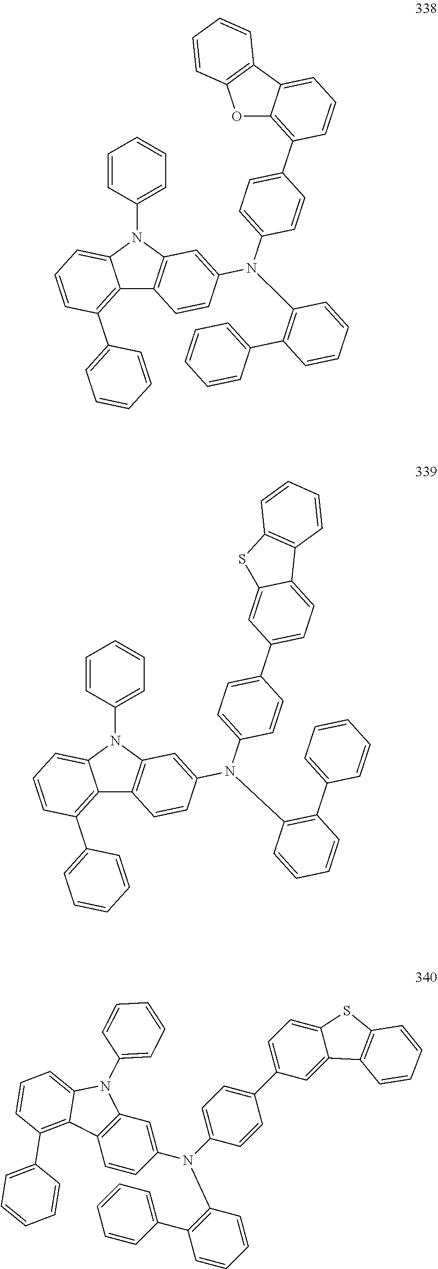 [SMILES: c1ccc(-c2ccccc2N(c2ccc(-c3cccc4c3oc3ccccc34)cc2)c2ccc3c4c(-c5ccccc5)cccc4n(-c4ccccc4)c3c2)cc1 ; c1ccc(-c2ccccc2N(c2ccc(-c3ccc4c(c3)sc3ccccc34)cc2)c2ccc3c4c(-c5ccccc5)cccc4n(-c4ccccc4)c3c2)cc1 ; c1ccc(-c2ccccc2N(c2ccc(-c3ccc4sc5ccccc5c4c3)cc2)c2ccc3c4c(-c5ccccc5)cccc4n(-c4ccccc4)c3c2)cc1]

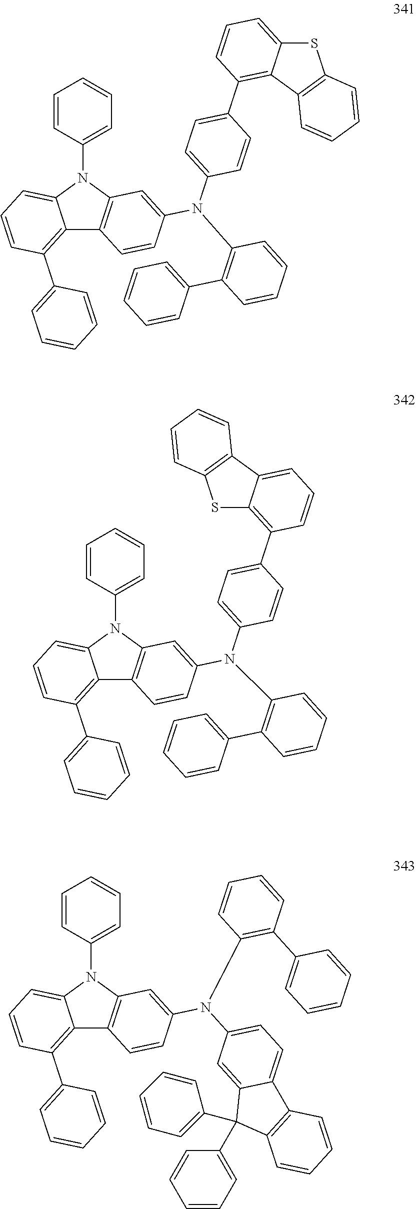 [SMILES: c1ccc(-c2ccccc2N(c2ccc(-c3cccc4sc5ccccc5c34)cc2)c2ccc3c4c(-c5ccccc5)cccc4n(-c4ccccc4)c3c2)cc1 ; c1ccc(-c2ccccc2N(c2ccc(-c3cccc4c3sc3ccccc34)cc2)c2ccc3c4c(-c5ccccc5)cccc4n(-c4ccccc4)c3c2)cc1 ; c1ccc(-c2ccccc2N(c2ccc3c(c2)C(c2ccccc2)(c2ccccc2)c2ccccc2-3)c2ccc3c4c(-c5ccccc5)cccc4n(-c4ccccc4)c3c2)cc1]

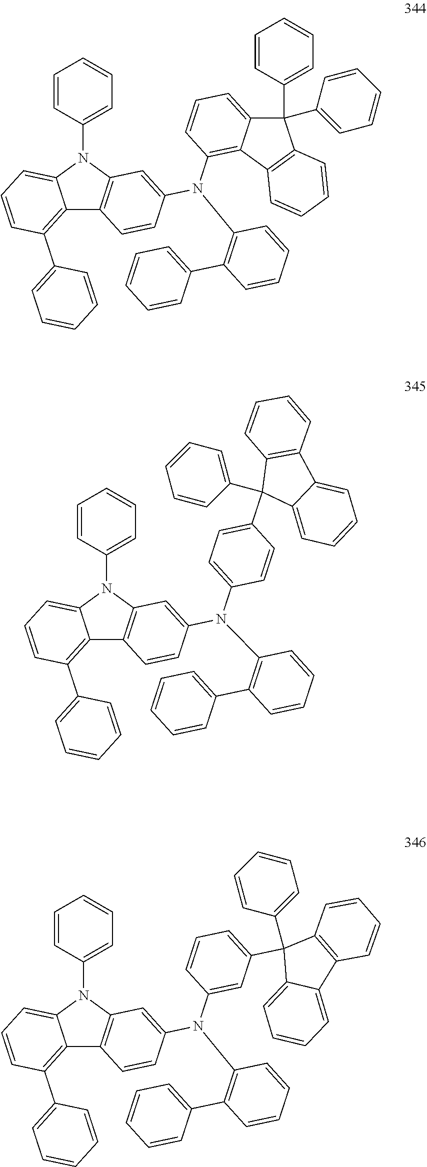 [SMILES: c1ccc(-c2ccccc2N(c2ccc3c4c(-c5ccccc5)cccc4n(-c4ccccc4)c3c2)c2cccc3c2-c2ccccc2C3(c2ccccc2)c2ccccc2)cc1 ; c1ccc(-c2ccccc2N(c2ccc(C3(c4ccccc4)c4ccccc4-c4ccccc43)cc2)c2ccc3c4c(-c5ccccc5)cccc4n(-c4ccccc4)c3c2)cc1 ; c1ccc(-c2ccccc2N(c2cccc(C3(c4ccccc4)c4ccccc4-c4ccccc43)c2)c2ccc3c4c(-c5ccccc5)cccc4n(-c4ccccc4)c3c2)cc1]

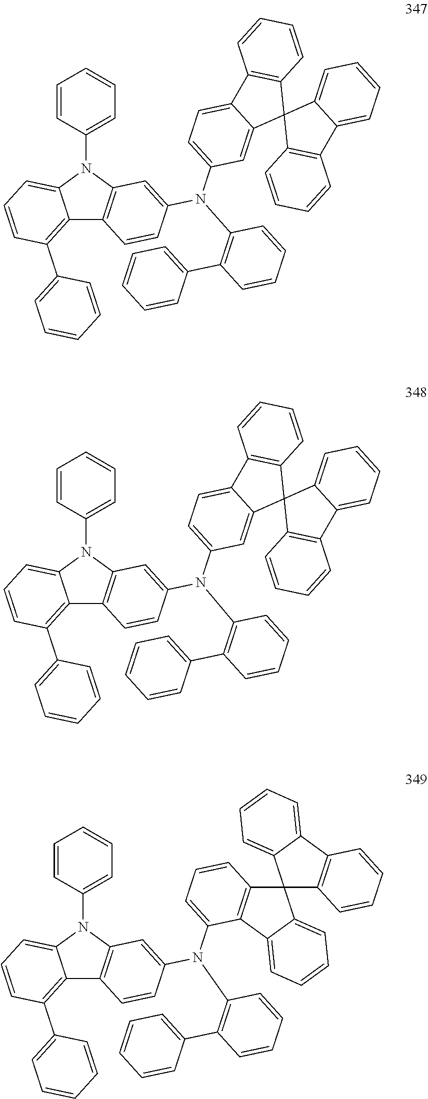 [SMILES: c1ccc(-c2ccccc2N(c2ccc3c(c2)C2(c4ccccc4-c4ccccc42)c2ccccc2-3)c2ccc3c4c(-c5ccccc5)cccc4n(-c4ccccc4)c3c2)cc1 ; c1ccc(-c2ccccc2N(c2ccc3c(c2)C2(c4ccccc4-c4ccccc42)c2ccccc2-3)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2cccc3c2-c2ccccc2C32c3ccccc3-c3ccccc32)cc1]

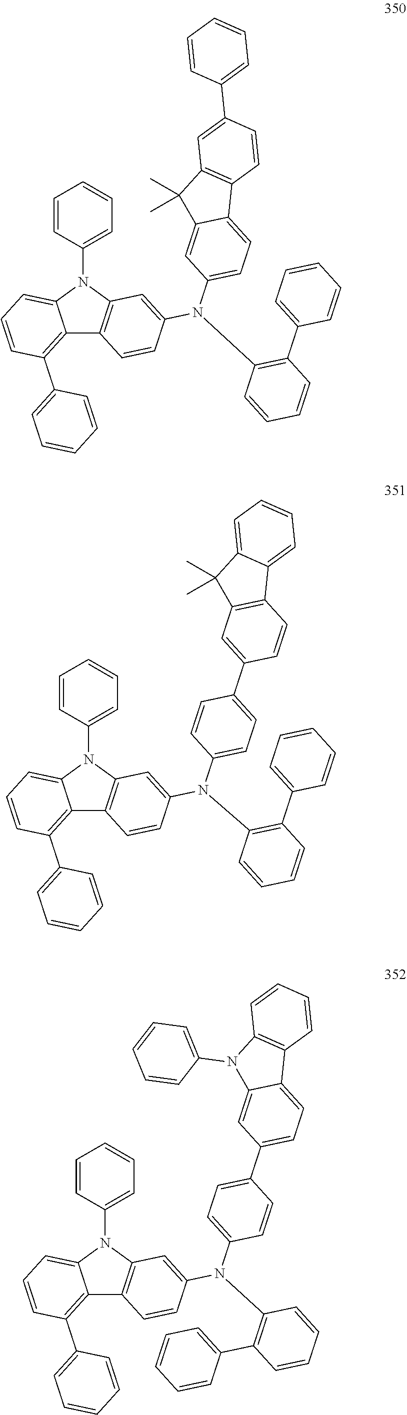 [SMILES: CC1(C)c2cc(-c3ccccc3)ccc2-c2ccc(N(c3ccc4c5c(-c6ccccc6)cccc5n(-c5ccccc5)c4c3)c3ccccc3-c3ccccc3)cc21 ; CC1(C)c2ccccc2-c2ccc(-c3ccc(N(c4ccc5c6c(-c7ccccc7)cccc6n(-c6ccccc6)c5c4)c4ccccc4-c4ccccc4)cc3)cc21 ; c1ccc(-c2ccccc2N(c2ccc(-c3ccc4c5ccccc5n(-c5ccccc5)c4c3)cc2)c2ccc3c4c(-c5ccccc5)cccc4n(-c4ccccc4)c3c2)cc1]

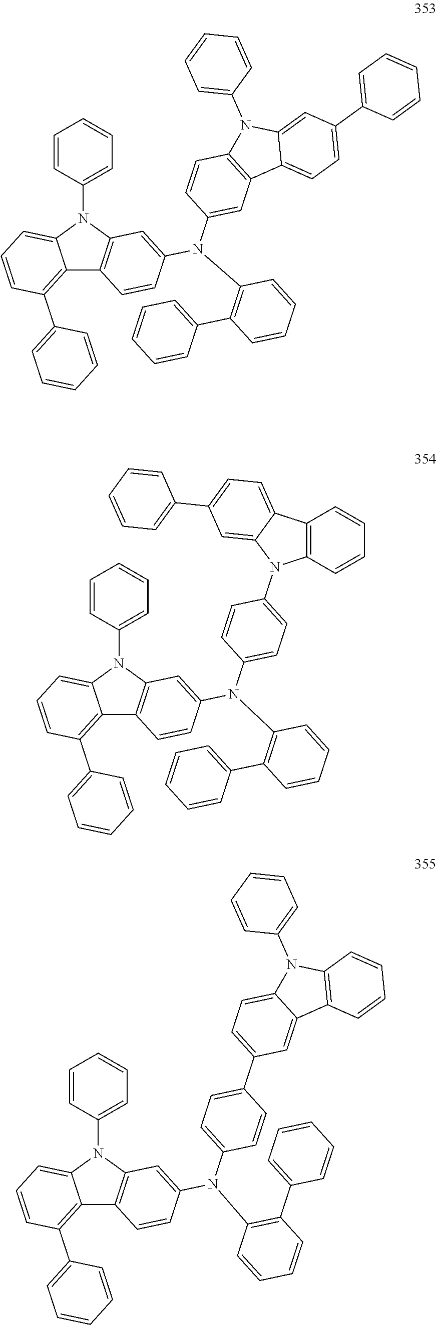 [SMILES: c1ccc(-c2ccc3c4cc(N(c5ccc6c7c(-c8ccccc8)cccc7n(-c7ccccc7)c6c5)c5ccccc5-c5ccccc5)ccc4n(-c4ccccc4)c3c2)cc1 ; c1ccc(-c2ccc3c4ccccc4n(-c4ccc(N(c5ccc6c7c(-c8ccccc8)cccc7n(-c7ccccc7)c6c5)c5ccccc5-c5ccccc5)cc4)c3c2)cc1 ; c1ccc(-c2ccccc2N(c2ccc(-c3ccc4c(c3)c3ccccc3n4-c3ccccc3)cc2)c2ccc3c4c(-c5ccccc5)cccc4n(-c4ccccc4)c3c2)cc1]

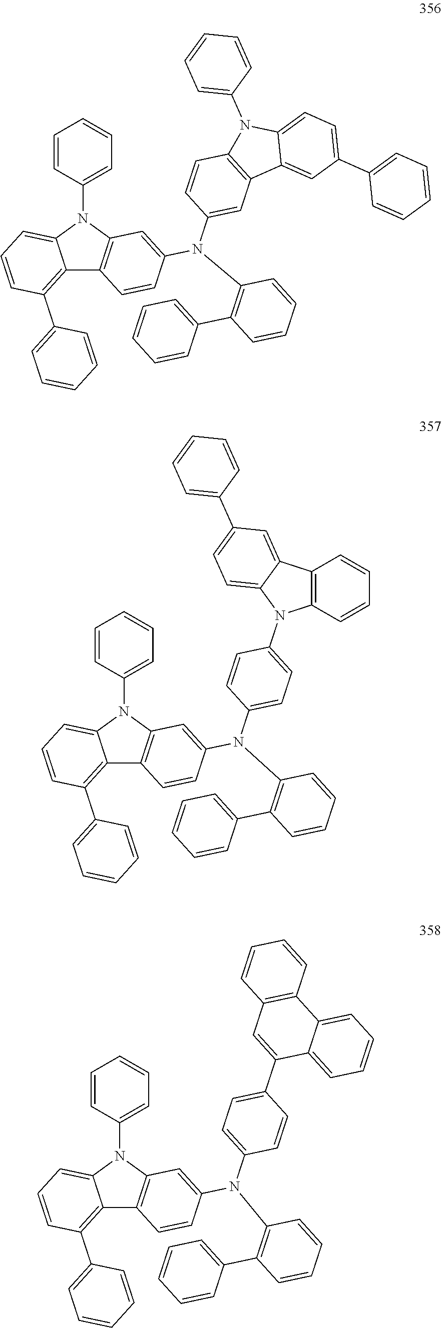 [SMILES: c1ccc(-c2ccc3c(c2)c2cc(N(c4ccc5c6c(-c7ccccc7)cccc6n(-c6ccccc6)c5c4)c4ccccc4-c4ccccc4)ccc2n3-c2ccccc2)cc1 ; c1ccc(-c2ccc3c(c2)c2ccccc2n3-c2ccc(N(c3ccc4c5c(-c6ccccc6)cccc5n(-c5ccccc5)c4c3)c3ccccc3-c3ccccc3)cc2)cc1 ; c1ccc(-c2ccccc2N(c2ccc(-c3cc4ccccc4c4ccccc34)cc2)c2ccc3c4c(-c5ccccc5)cccc4n(-c4ccccc4)c3c2)cc1]

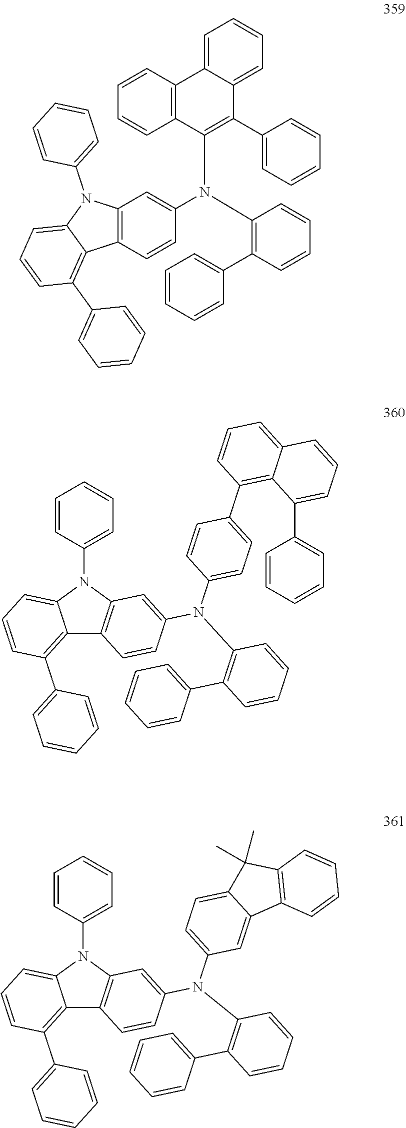 [SMILES: c1ccc(-c2ccccc2N(c2ccc3c4c(-c5ccccc5)cccc4n(-c4ccccc4)c3c2)c2c(-c3ccccc3)c3ccccc3c3ccccc23)cc1 ; c1ccc(-c2ccccc2N(c2ccc(-c3cccc4cccc(-c5ccccc5)c34)cc2)c2ccc3c4c(-c5ccccc5)cccc4n(-c4ccccc4)c3c2)cc1 ; CC1(C)c2ccccc2-c2cc(N(c3ccc4c5c(-c6ccccc6)cccc5n(-c5ccccc5)c4c3)c3ccccc3-c3ccccc3)ccc21]

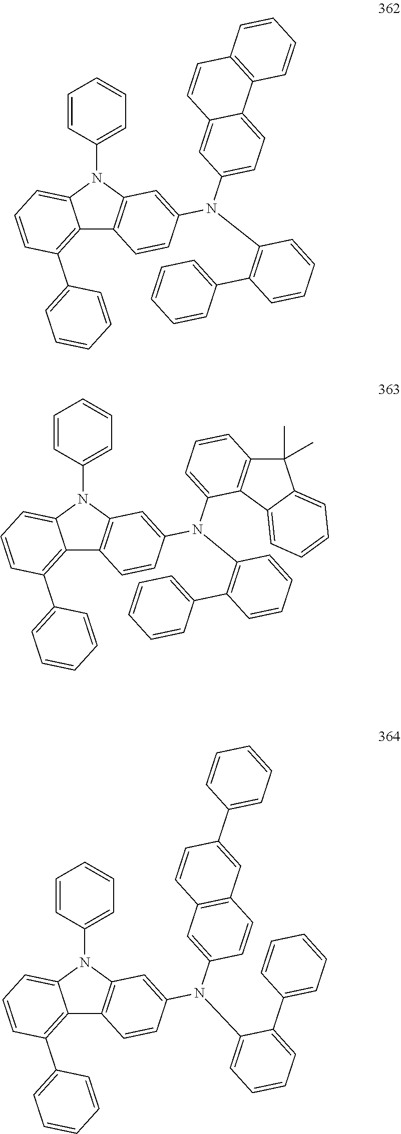 [SMILES: c1ccc(-c2ccccc2N(c2ccc3c(ccc4ccccc43)c2)c2ccc3c4c(-c5ccccc5)cccc4n(-c4ccccc4)c3c2)cc1 ; CC1(C)c2ccccc2-c2c(N(c3ccc4c5c(-c6ccccc6)cccc5n(-c5ccccc5)c4c3)c3ccccc3-c3ccccc3)cccc21 ; c1ccc(-c2ccc3cc(N(c4ccc5c6c(-c7ccccc7)cccc6n(-c6ccccc6)c5c4)c4ccccc4-c4ccccc4)ccc3c2)cc1]

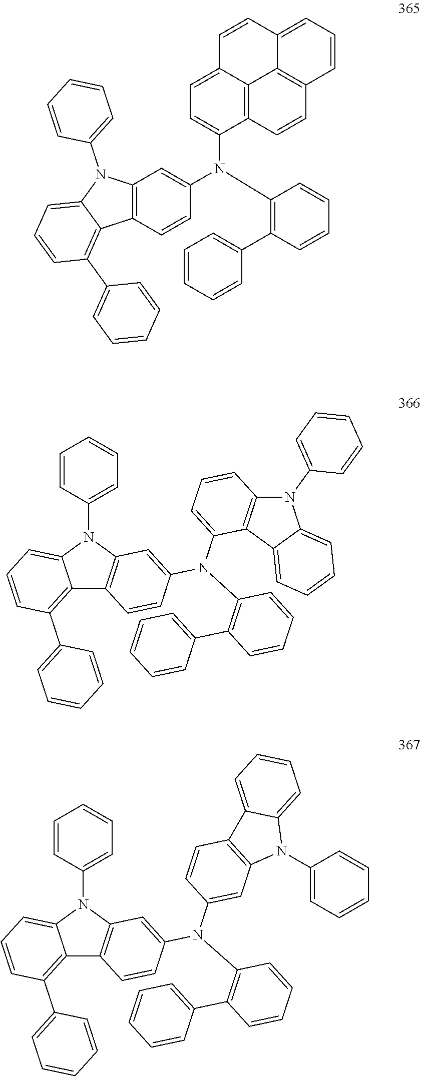 [SMILES: c1ccc(-c2ccccc2N(c2ccc3c4c(-c5ccccc5)cccc4n(-c4ccccc4)c3c2)c2ccc3ccc4cccc5ccc2c3c45)cc1 ; c1ccc(-c2ccccc2N(c2ccc3c4c(-c5ccccc5)cccc4n(-c4ccccc4)c3c2)c2cccc3c2c2ccccc2n3-c2ccccc2)cc1 ; c1ccc(-c2ccccc2N(c2ccc3c4ccccc4n(-c4ccccc4)c3c2)c2ccc3c4c(-c5ccccc5)cccc4n(-c4ccccc4)c3c2)cc1]

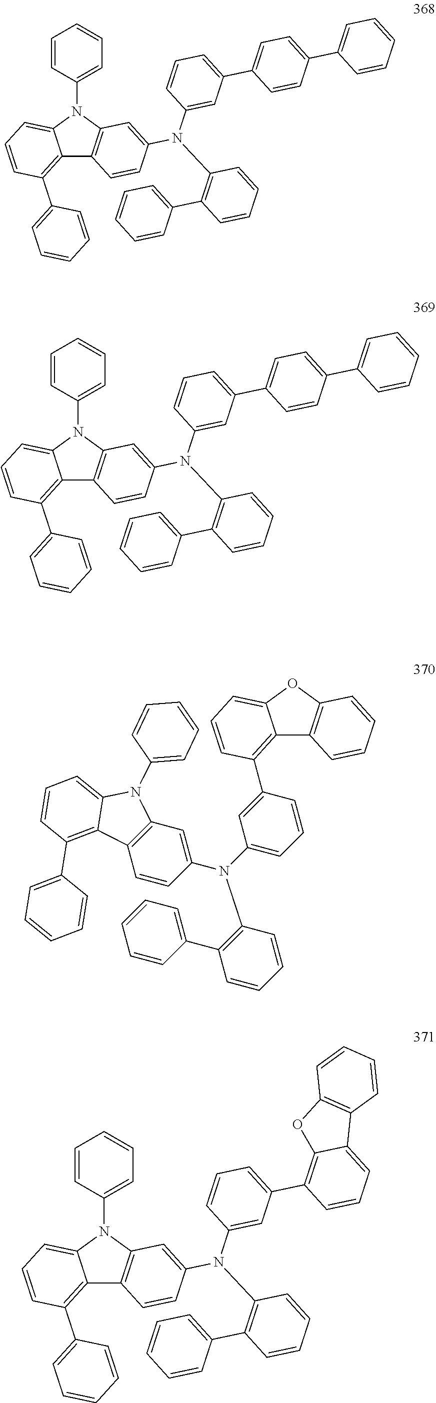 [SMILES: c1ccc(-c2ccc(-c3cccc(N(c4ccc5c6c(-c7ccccc7)cccc6n(-c6ccccc6)c5c4)c4ccccc4-c4ccccc4)c3)cc2)cc1 ; c1ccc(-c2ccc(-c3cccc(N(c4ccc5c6c(-c7ccccc7)cccc6n(-c6ccccc6)c5c4)c4ccccc4-c4ccccc4)c3)cc2)cc1 ; c1ccc(-c2ccccc2N(c2cccc(-c3cccc4oc5ccccc5c34)c2)c2ccc3c4c(-c5ccccc5)cccc4n(-c4ccccc4)c3c2)cc1 ; c1ccc(-c2ccccc2N(c2cccc(-c3cccc4c3oc3ccccc34)c2)c2ccc3c4c(-c5ccccc5)cccc4n(-c4ccccc4)c3c2)cc1]

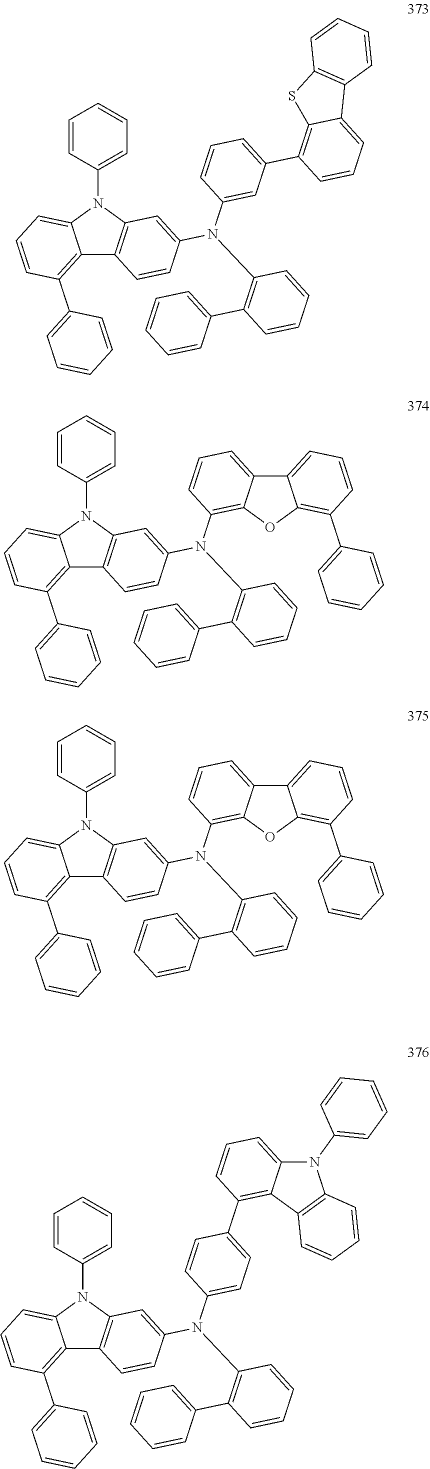

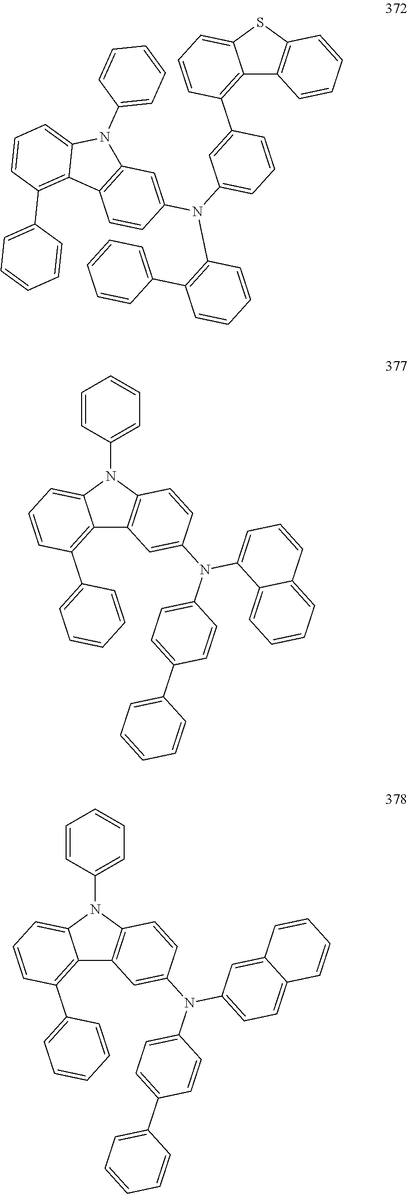 [SMILES: c1ccc(-c2ccccc2N(c2cccc(-c3cccc4sc5ccccc5c34)c2)c2ccc3c4c(-c5ccccc5)cccc4n(-c4ccccc4)c3c2)cc1 ; c1ccc(-c2ccc(N(c3ccc4c(c3)c3c(-c5ccccc5)cccc3n4-c3ccccc3)c3cccc4ccccc34)cc2)cc1 ; c1ccc(-c2ccc(N(c3ccc4ccccc4c3)c3ccc4c(c3)c3c(-c5ccccc5)cccc3n4-c3ccccc3)cc2)cc1]

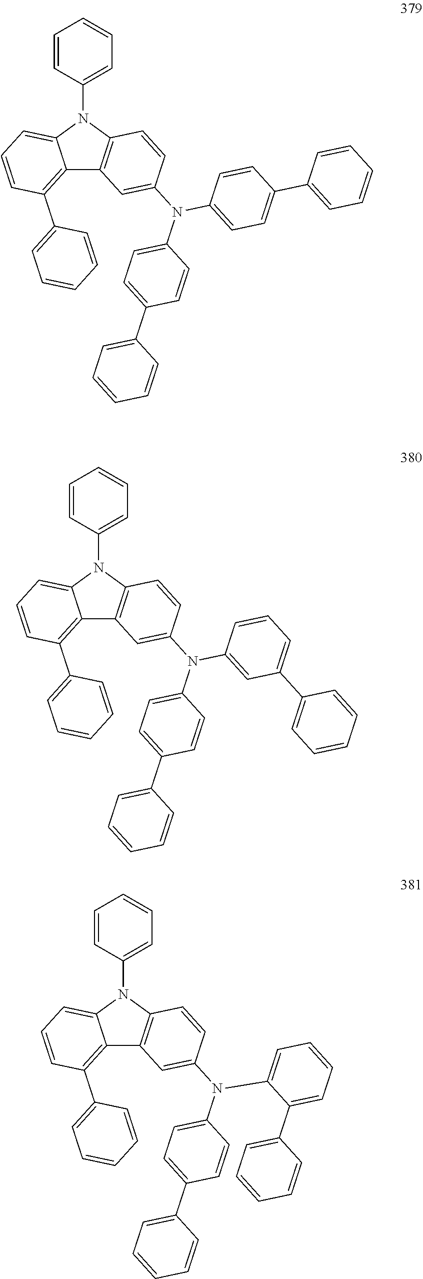

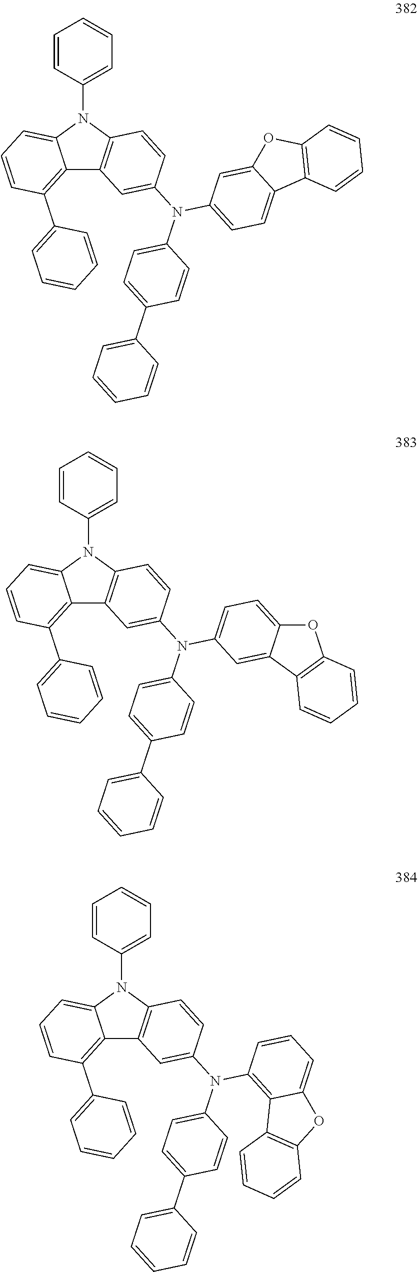 [SMILES: c1ccc(-c2ccc(N(c3ccc4c(c3)oc3ccccc34)c3ccc4c(c3)c3c(-c5ccccc5)cccc3n4-c3ccccc3)cc2)cc1 ; c1ccc(-c2ccc(N(c3ccc4oc5ccccc5c4c3)c3ccc4c(c3)c3c(-c5ccccc5)cccc3n4-c3ccccc3)cc2)cc1 ; c1ccc(-c2ccc(N(c3ccc4c(c3)c3c(-c5ccccc5)cccc3n4-c3ccccc3)c3cccc4oc5ccccc5c34)cc2)cc1]

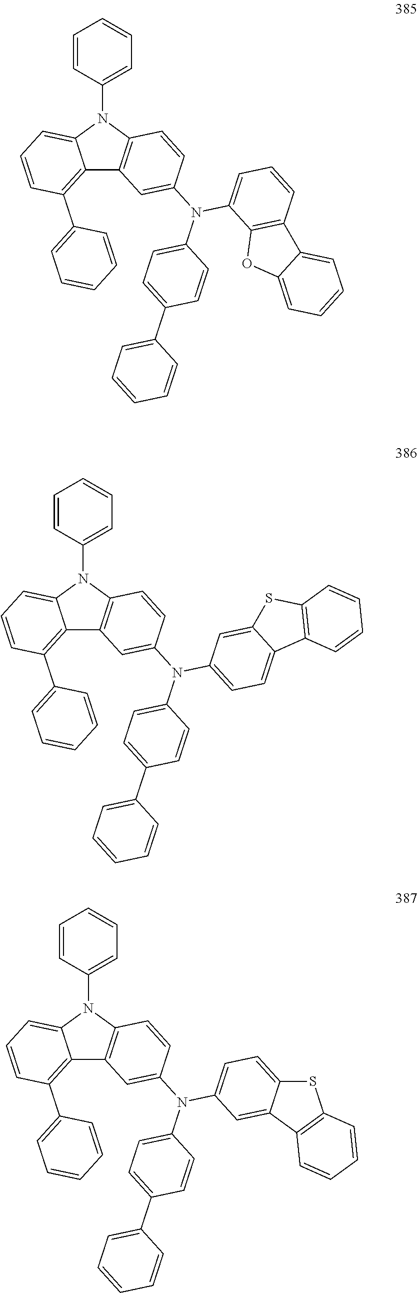 [SMILES: c1ccc(-c2ccc(N(c3ccc4c(c3)c3c(-c5ccccc5)cccc3n4-c3ccccc3)c3cccc4c3oc3ccccc34)cc2)cc1 ; c1ccc(-c2ccc(N(c3ccc4c(c3)sc3ccccc34)c3ccc4c(c3)c3c(-c5ccccc5)cccc3n4-c3ccccc3)cc2)cc1 ; c1ccc(-c2ccc(N(c3ccc4sc5ccccc5c4c3)c3ccc4c(c3)c3c(-c5ccccc5)cccc3n4-c3ccccc3)cc2)cc1]

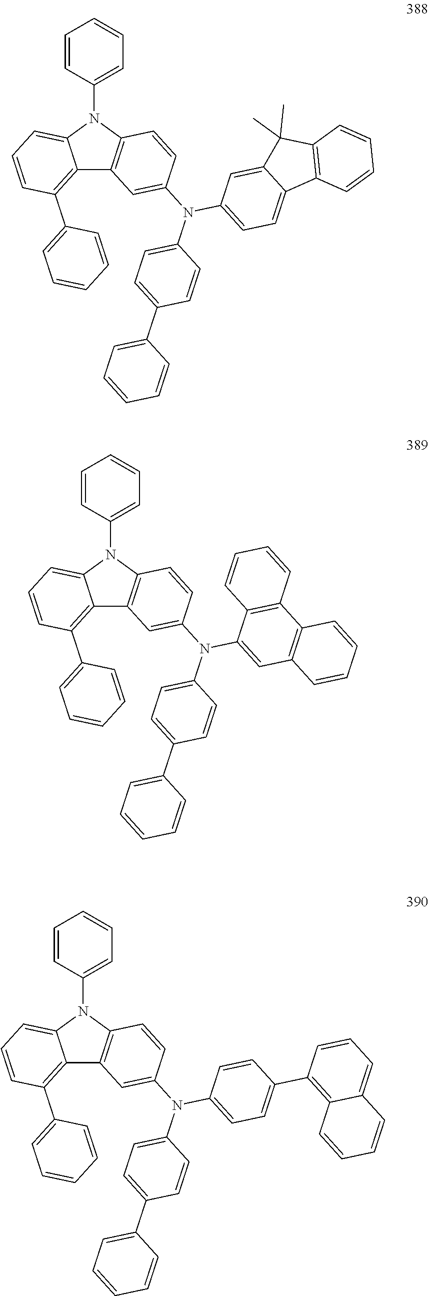 [SMILES: CC1(C)c2ccccc2-c2ccc(N(c3ccc(-c4ccccc4)cc3)c3ccc4c(c3)c3c(-c5ccccc5)cccc3n4-c3ccccc3)cc21 ; c1ccc(-c2ccc(N(c3ccc4c(c3)c3c(-c5ccccc5)cccc3n4-c3ccccc3)c3cc4ccccc4c4ccccc34)cc2)cc1 ; c1ccc(-c2ccc(N(c3ccc(-c4cccc5ccccc45)cc3)c3ccc4c(c3)c3c(-c5ccccc5)cccc3n4-c3ccccc3)cc2)cc1]

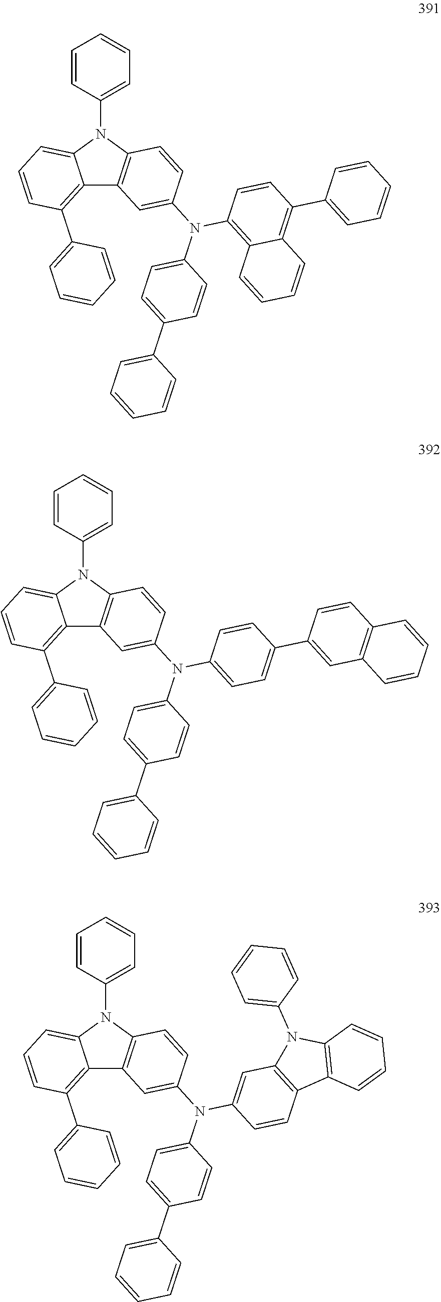 [SMILES: c1ccc(-c2ccc(N(c3ccc4c(c3)c3c(-c5ccccc5)cccc3n4-c3ccccc3)c3ccc(-c4ccccc4)c4ccccc34)cc2)cc1 ; c1ccc(-c2ccc(N(c3ccc(-c4ccc5ccccc5c4)cc3)c3ccc4c(c3)c3c(-c5ccccc5)cccc3n4-c3ccccc3)cc2)cc1 ; c1ccc(-c2ccc(N(c3ccc4c(c3)c3c(-c5ccccc5)cccc3n4-c3ccccc3)c3ccc4c5ccccc5n(-c5ccccc5)c4c3)cc2)cc1]

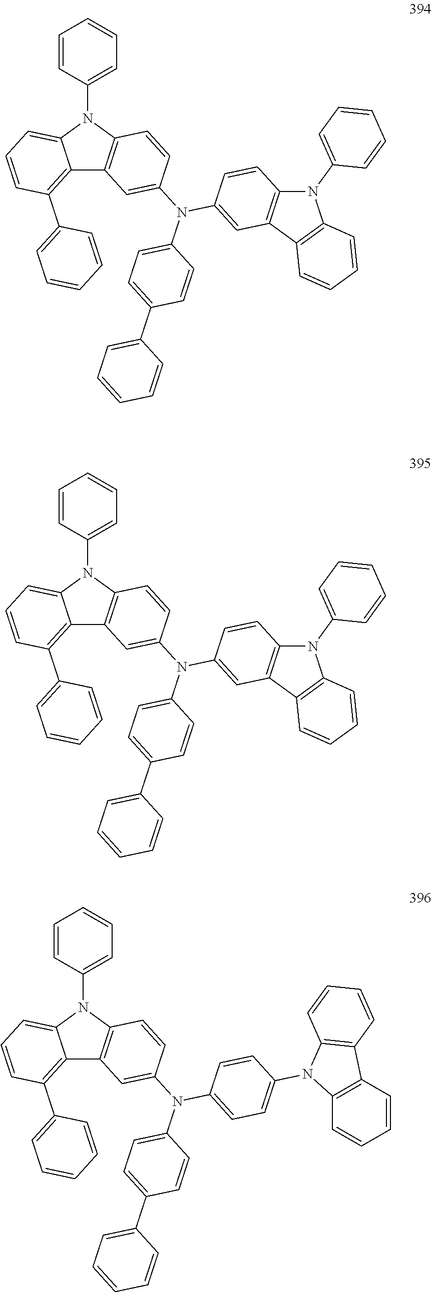 [SMILES: c1ccc(-c2ccc(N(c3ccc4c(c3)c3ccccc3n4-c3ccccc3)c3ccc4c(c3)c3c(-c5ccccc5)cccc3n4-c3ccccc3)cc2)cc1 ; c1ccc(-c2ccc(N(c3ccc4c(c3)c3ccccc3n4-c3ccccc3)c3ccc4c(c3)c3c(-c5ccccc5)cccc3n4-c3ccccc3)cc2)cc1 ; c1ccc(-c2ccc(N(c3ccc(-n4c5ccccc5c5ccccc54)cc3)c3ccc4c(c3)c3c(-c5ccccc5)cccc3n4-c3ccccc3)cc2)cc1]

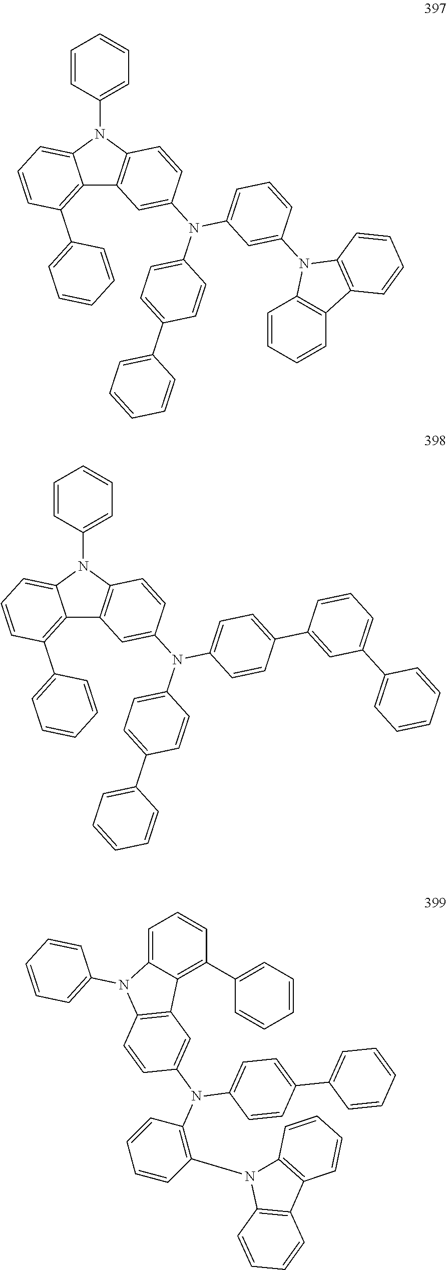 [SMILES: c1ccc(-c2ccc(N(c3cccc(-n4c5ccccc5c5ccccc54)c3)c3ccc4c(c3)c3c(-c5ccccc5)cccc3n4-c3ccccc3)cc2)cc1 ; c1ccc(-c2ccc(N(c3ccc(-c4cccc(-c5ccccc5)c4)cc3)c3ccc4c(c3)c3c(-c5ccccc5)cccc3n4-c3ccccc3)cc2)cc1 ; c1ccc(-c2ccc(N(c3ccc4c(c3)c3c(-c5ccccc5)cccc3n4-c3ccccc3)c3ccccc3-n3c4ccccc4c4ccccc43)cc2)cc1]

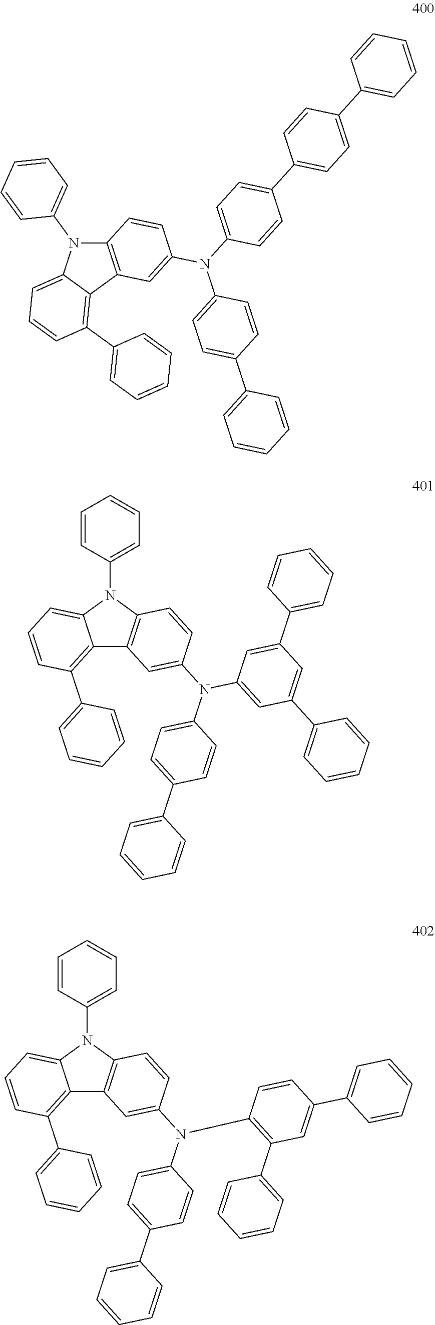 [SMILES: c1ccc(-c2ccc(-c3ccc(N(c4ccc(-c5ccccc5)cc4)c4ccc5c(c4)c4c(-c6ccccc6)cccc4n5-c4ccccc4)cc3)cc2)cc1 ; c1ccc(-c2ccc(N(c3cc(-c4ccccc4)cc(-c4ccccc4)c3)c3ccc4c(c3)c3c(-c5ccccc5)cccc3n4-c3ccccc3)cc2)cc1 ; c1ccc(-c2ccc(N(c3ccc4c(c3)c3c(-c5ccccc5)cccc3n4-c3ccccc3)c3ccc(-c4ccccc4)cc3-c3ccccc3)cc2)cc1]

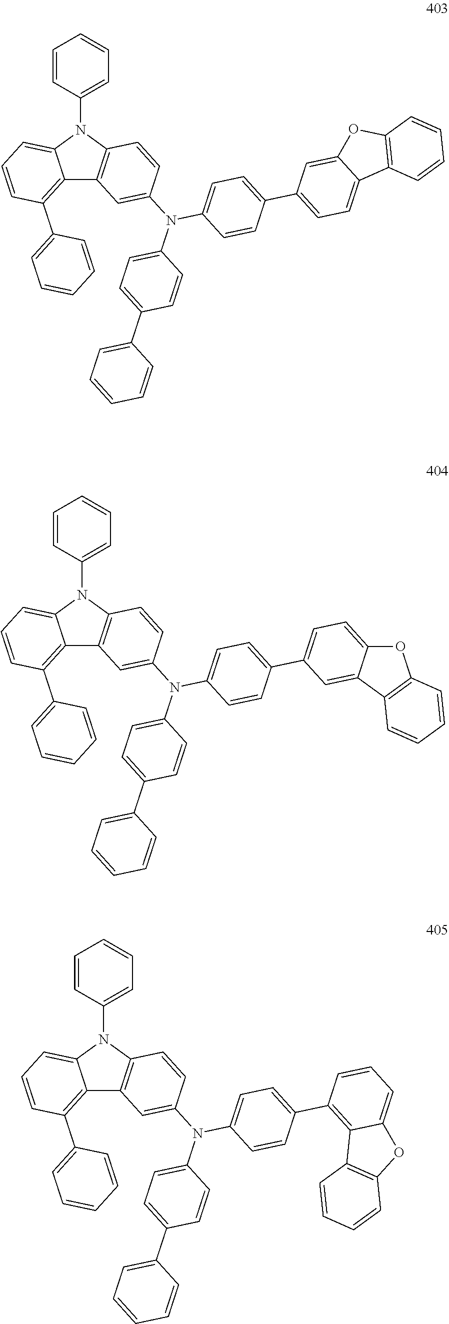 [SMILES: c1ccc(-c2ccc(N(c3ccc(-c4ccc5c(c4)oc4ccccc45)cc3)c3ccc4c(c3)c3c(-c5ccccc5)cccc3n4-c3ccccc3)cc2)cc1 ; c1ccc(-c2ccc(N(c3ccc(-c4ccc5oc6ccccc6c5c4)cc3)c3ccc4c(c3)c3c(-c5ccccc5)cccc3n4-c3ccccc3)cc2)cc1 ; c1ccc(-c2ccc(N(c3ccc(-c4cccc5oc6ccccc6c45)cc3)c3ccc4c(c3)c3c(-c5ccccc5)cccc3n4-c3ccccc3)cc2)cc1]

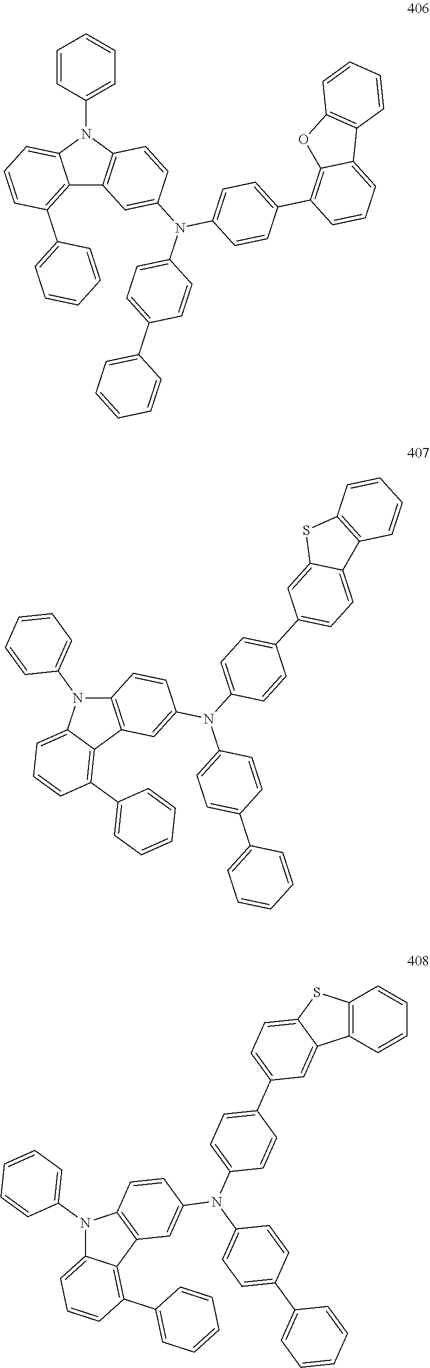 [SMILES: c1ccc(-c2ccc(N(c3ccc(-c4cccc5c4oc4ccccc45)cc3)c3ccc4c(c3)c3c(-c5ccccc5)cccc3n4-c3ccccc3)cc2)cc1 ; c1ccc(-c2ccc(N(c3ccc(-c4ccc5c(c4)sc4ccccc45)cc3)c3ccc4c(c3)c3c(-c5ccccc5)cccc3n4-c3ccccc3)cc2)cc1 ; c1ccc(-c2ccc(N(c3ccc(-c4ccc5sc6ccccc6c5c4)cc3)c3ccc4c(c3)c3c(-c5ccccc5)cccc3n4-c3ccccc3)cc2)cc1]

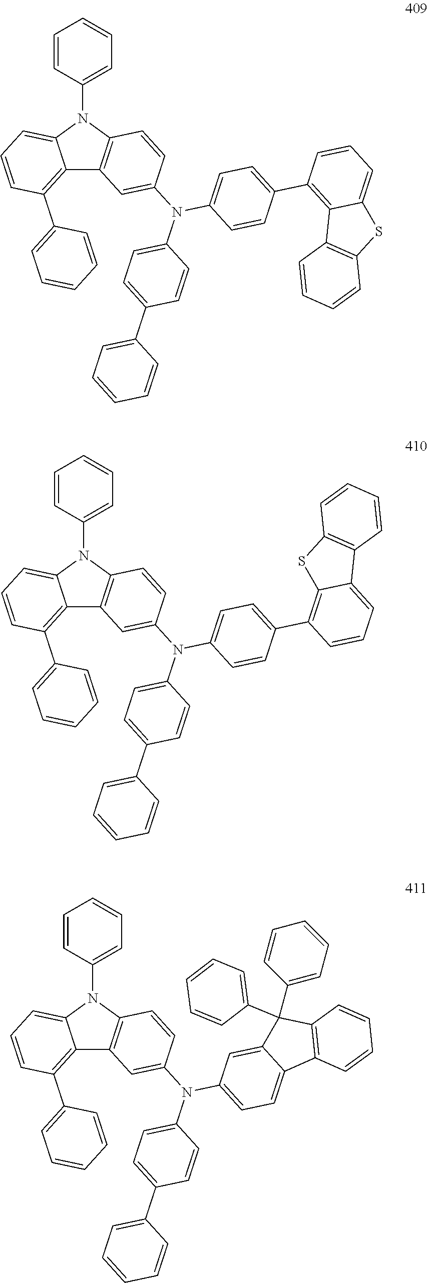 [SMILES: c1ccc(-c2ccc(N(c3ccc(-c4cccc5sc6ccccc6c45)cc3)c3ccc4c(c3)c3c(-c5ccccc5)cccc3n4-c3ccccc3)cc2)cc1 ; c1ccc(-c2ccc(N(c3ccc(-c4cccc5c4sc4ccccc45)cc3)c3ccc4c(c3)c3c(-c5ccccc5)cccc3n4-c3ccccc3)cc2)cc1 ; c1ccc(-c2ccc(N(c3ccc4c(c3)C(c3ccccc3)(c3ccccc3)c3ccccc3-4)c3ccc4c(c3)c3c(-c5ccccc5)cccc3n4-c3ccccc3)cc2)cc1]

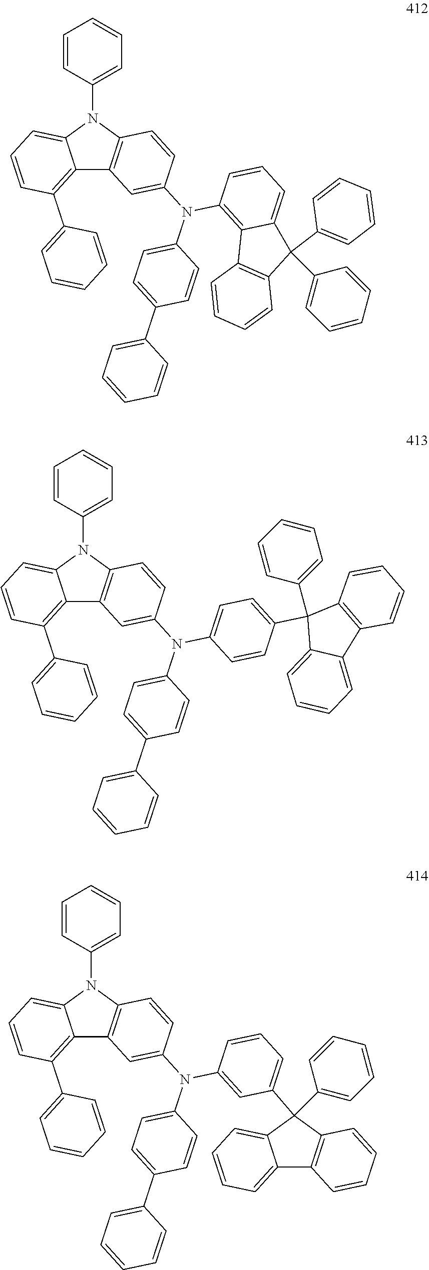 [SMILES: c1ccc(-c2ccc(N(c3ccc4c(c3)c3c(-c5ccccc5)cccc3n4-c3ccccc3)c3cccc4c3-c3ccccc3C4(c3ccccc3)c3ccccc3)cc2)cc1 ; c1ccc(-c2ccc(N(c3ccc(C4(c5ccccc5)c5ccccc5-c5ccccc54)cc3)c3ccc4c(c3)c3c(-c5ccccc5)cccc3n4-c3ccccc3)cc2)cc1 ; c1ccc(-c2ccc(N(c3cccc(C4(c5ccccc5)c5ccccc5-c5ccccc54)c3)c3ccc4c(c3)c3c(-c5ccccc5)cccc3n4-c3ccccc3)cc2)cc1]

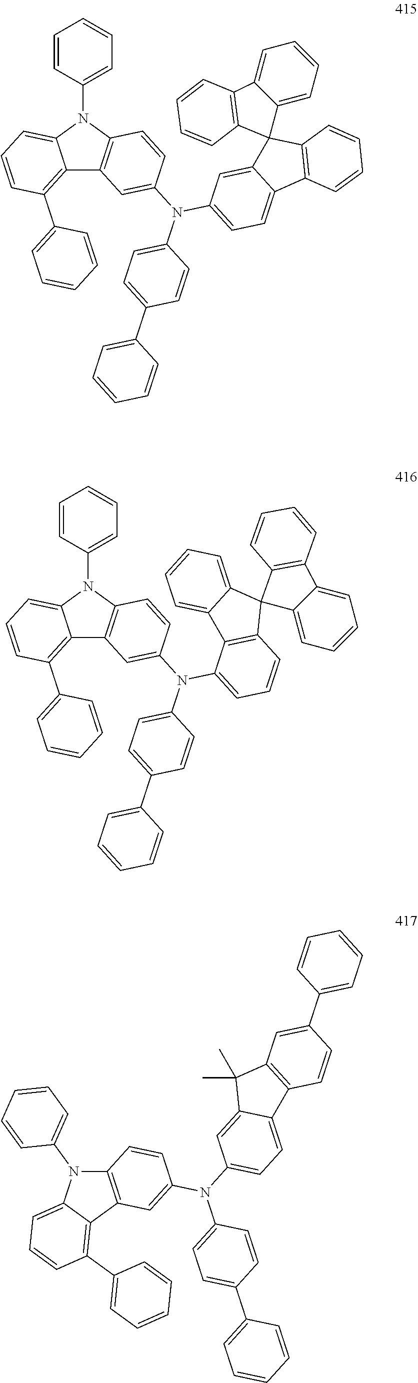 [SMILES: c1ccc(-c2ccc(N(c3ccc4c(c3)C3(c5ccccc5-c5ccccc53)c3ccccc3-4)c3ccc4c(c3)c3c(-c5ccccc5)cccc3n4-c3ccccc3)cc2)cc1 ; c1ccc(-c2ccc(N(c3ccc4c(c3)c3c(-c5ccccc5)cccc3n4-c3ccccc3)c3cccc4c3-c3ccccc3C43c4ccccc4-c4ccccc43)cc2)cc1 ; CC1(C)c2cc(-c3ccccc3)ccc2-c2ccc(N(c3ccc(-c4ccccc4)cc3)c3ccc4c(c3)c3c(-c5ccccc5)cccc3n4-c3ccccc3)cc21]

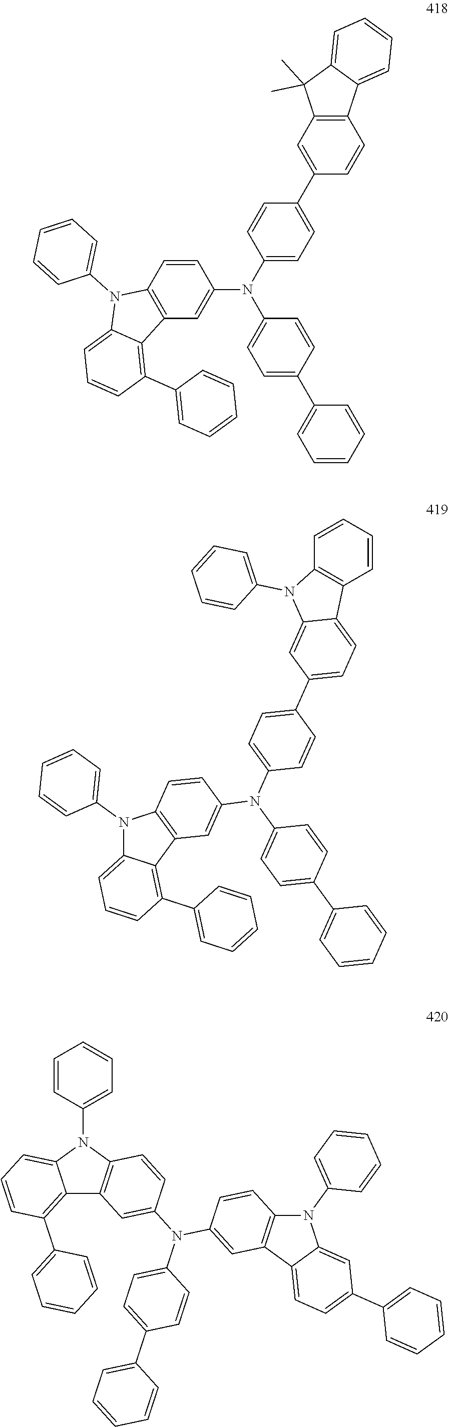 [SMILES: CC1(C)c2ccccc2-c2ccc(-c3ccc(N(c4ccc(-c5ccccc5)cc4)c4ccc5c(c4)c4c(-c6ccccc6)cccc4n5-c4ccccc4)cc3)cc21 ; c1ccc(-c2ccc(N(c3ccc(-c4ccc5c6ccccc6n(-c6ccccc6)c5c4)cc3)c3ccc4c(c3)c3c(-c5ccccc5)cccc3n4-c3ccccc3)cc2)cc1 ; c1ccc(-c2ccc(N(c3ccc4c(c3)c3ccc(-c5ccccc5)cc3n4-c3ccccc3)c3ccc4c(c3)c3c(-c5ccccc5)cccc3n4-c3ccccc3)cc2)cc1]

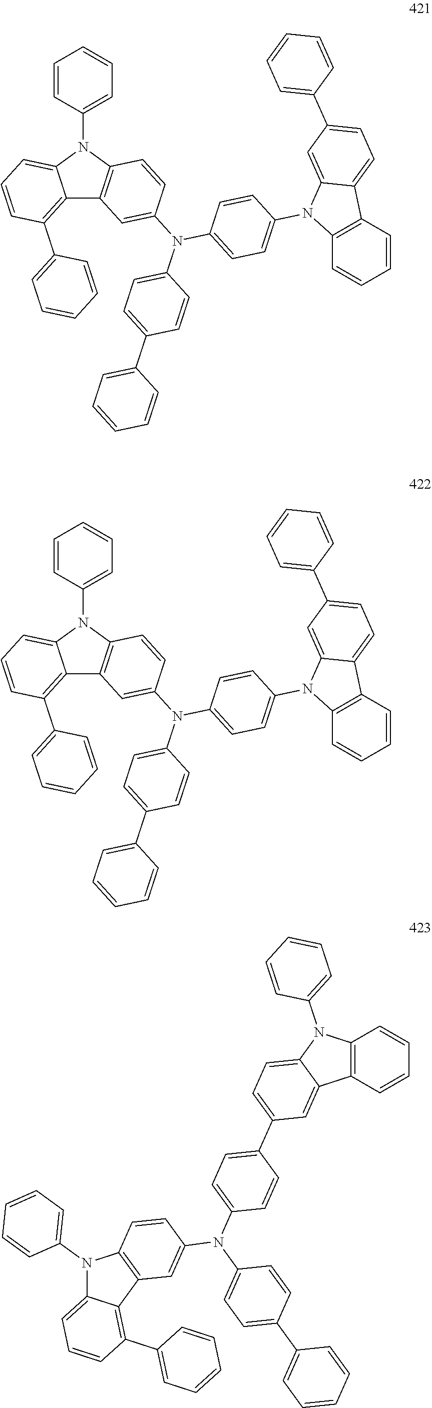 [SMILES: c1ccc(-c2ccc(N(c3ccc(-n4c5ccccc5c5ccc(-c6ccccc6)cc54)cc3)c3ccc4c(c3)c3c(-c5ccccc5)cccc3n4-c3ccccc3)cc2)cc1 ; c1ccc(-c2ccc(N(c3ccc(-n4c5ccccc5c5ccc(-c6ccccc6)cc54)cc3)c3ccc4c(c3)c3c(-c5ccccc5)cccc3n4-c3ccccc3)cc2)cc1 ; c1ccc(-c2ccc(N(c3ccc(-c4ccc5c(c4)c4ccccc4n5-c4ccccc4)cc3)c3ccc4c(c3)c3c(-c5ccccc5)cccc3n4-c3ccccc3)cc2)cc1]

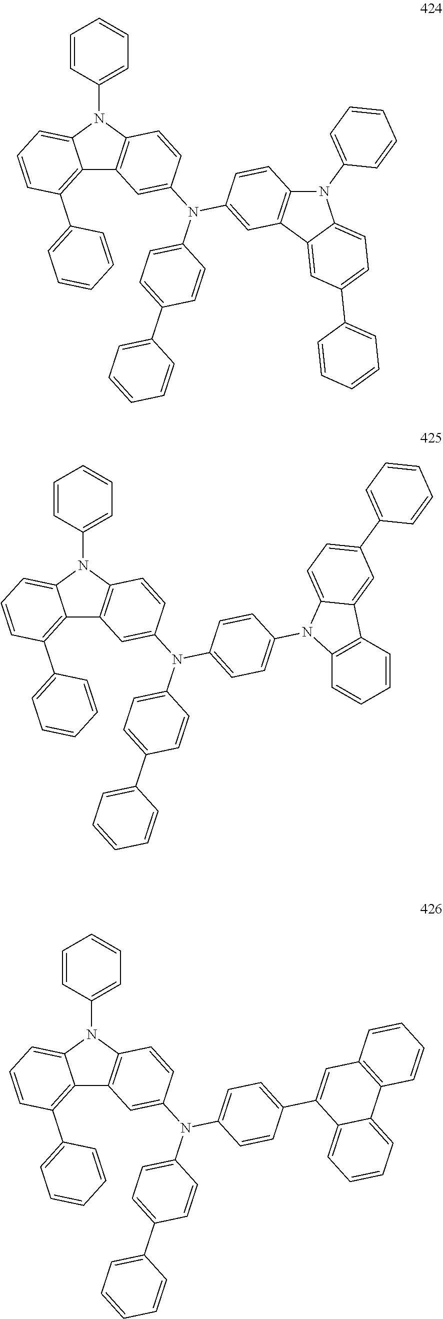 [SMILES: c1ccc(-c2ccc(N(c3ccc4c(c3)c3cc(-c5ccccc5)ccc3n4-c3ccccc3)c3ccc4c(c3)c3c(-c5ccccc5)cccc3n4-c3ccccc3)cc2)cc1 ; c1ccc(-c2ccc(N(c3ccc(-n4c5ccccc5c5cc(-c6ccccc6)ccc54)cc3)c3ccc4c(c3)c3c(-c5ccccc5)cccc3n4-c3ccccc3)cc2)cc1 ; c1ccc(-c2ccc(N(c3ccc(-c4cc5ccccc5c5ccccc45)cc3)c3ccc4c(c3)c3c(-c5ccccc5)cccc3n4-c3ccccc3)cc2)cc1]

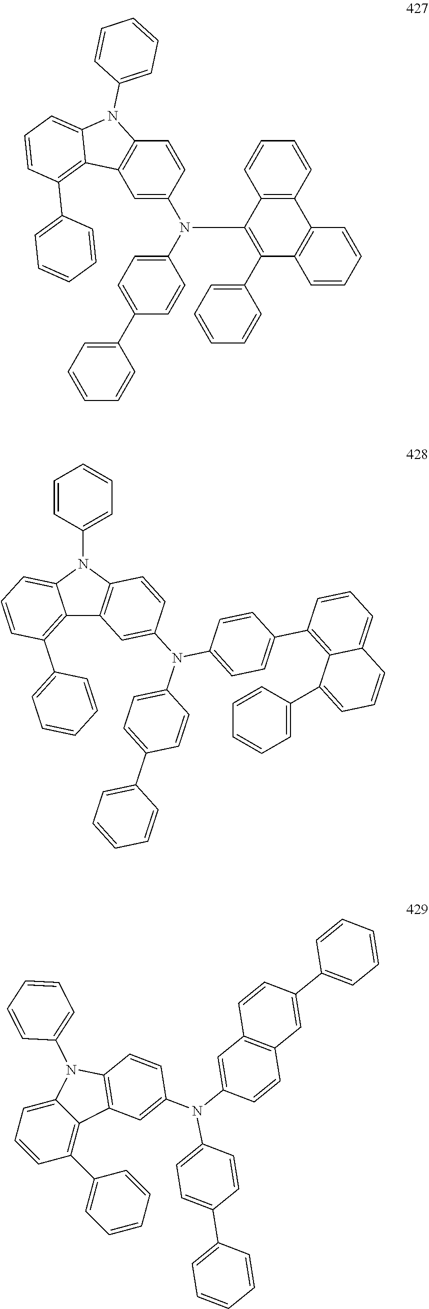 [SMILES: c1ccc(-c2ccc(N(c3ccc4c(c3)c3c(-c5ccccc5)cccc3n4-c3ccccc3)c3c(-c4ccccc4)c4ccccc4c4ccccc34)cc2)cc1 ; c1ccc(-c2ccc(N(c3ccc(-c4cccc5cccc(-c6ccccc6)c45)cc3)c3ccc4c(c3)c3c(-c5ccccc5)cccc3n4-c3ccccc3)cc2)cc1 ; c1ccc(-c2ccc(N(c3ccc4cc(-c5ccccc5)ccc4c3)c3ccc4c(c3)c3c(-c5ccccc5)cccc3n4-c3ccccc3)cc2)cc1]

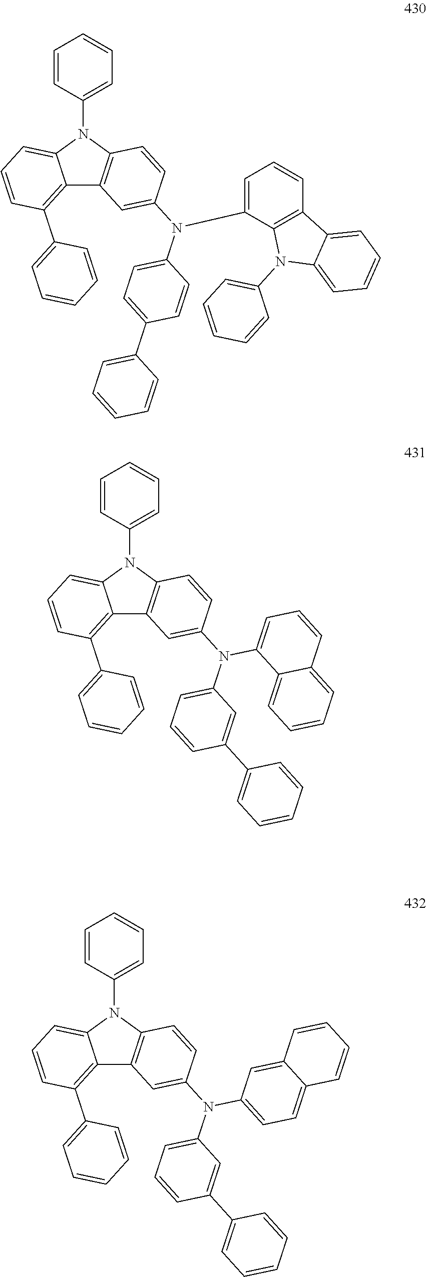

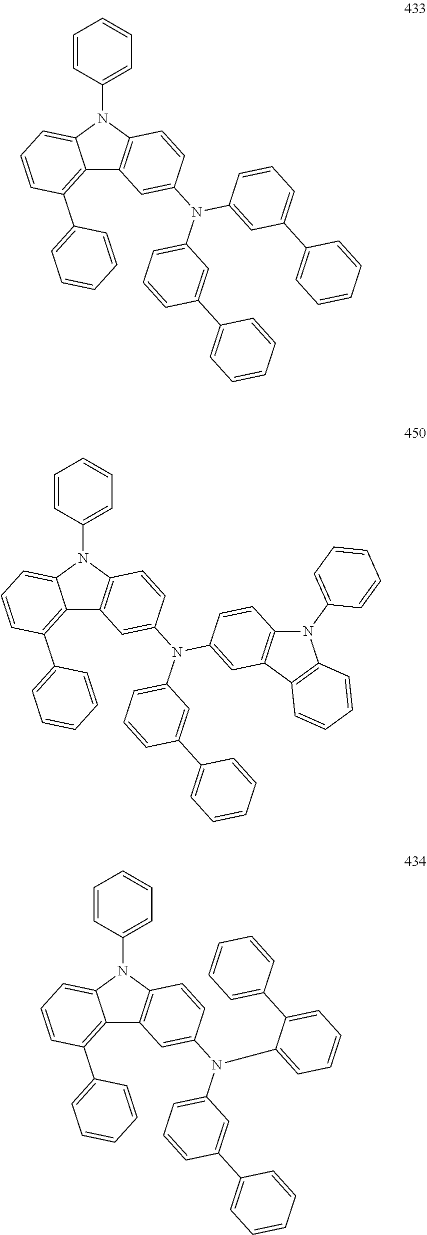 [SMILES: c1ccc(-c2cccc(N(c3cccc(-c4ccccc4)c3)c3ccc4c(c3)c3c(-c5ccccc5)cccc3n4-c3ccccc3)c2)cc1 ; c1ccc(-c2cccc(N(c3ccc4c(c3)c3ccccc3n4-c3ccccc3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3ccccc3-c3ccccc3)c2)cc1]

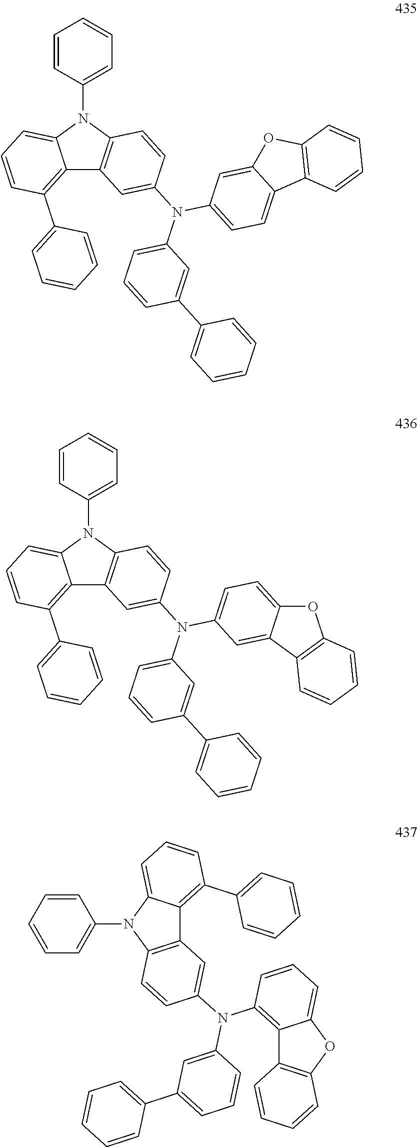 [SMILES: c1ccc(-c2cccc(N(c3ccc4c(c3)oc3ccccc34)c3ccc4c(c3)c3c(-c5ccccc5)cccc3n4-c3ccccc3)c2)cc1 ; c1ccc(-c2cccc(N(c3ccc4oc5ccccc5c4c3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3cccc4oc5ccccc5c34)c2)cc1]

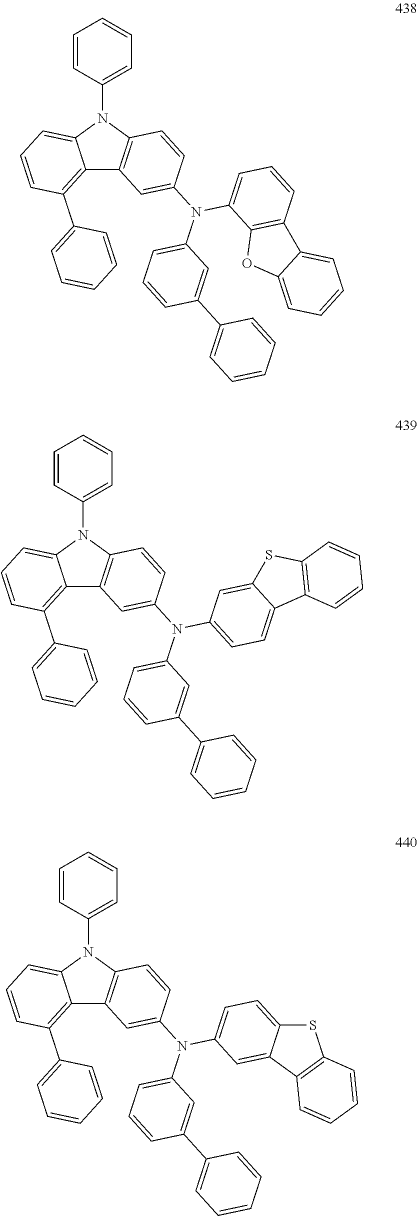 [SMILES: c1ccc(-c2cccc(N(c3ccc4c(c3)c3c(-c5ccccc5)cccc3n4-c3ccccc3)c3cccc4c3oc3ccccc34)c2)cc1 ; c1ccc(-c2cccc(N(c3ccc4c(c3)sc3ccccc34)c3ccc4c(c3)c3c(-c5ccccc5)cccc3n4-c3ccccc3)c2)cc1 ; c1ccc(-c2cccc(N(c3ccc4sc5ccccc5c4c3)c3ccc4c(c3)c3c(-c5ccccc5)cccc3n4-c3ccccc3)c2)cc1]

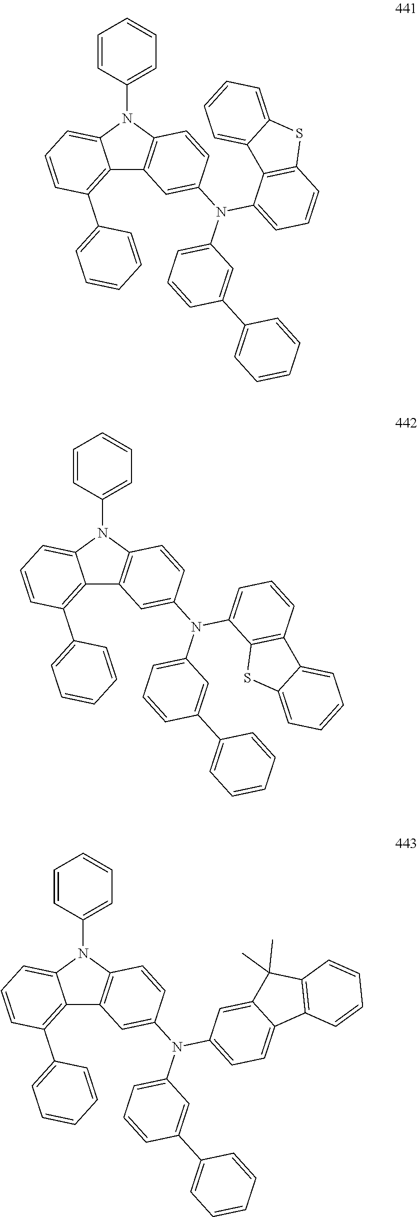 [SMILES: c1ccc(-c2cccc(N(c3ccc4c(c3)c3c(-c5ccccc5)cccc3n4-c3ccccc3)c3cccc4sc5ccccc5c34)c2)cc1 ; c1ccc(-c2cccc(N(c3ccc4c(c3)c3c(-c5ccccc5)cccc3n4-c3ccccc3)c3cccc4c3sc3ccccc34)c2)cc1 ; CC1(C)c2ccccc2-c2ccc(N(c3cccc(-c4ccccc4)c3)c3ccc4c(c3)c3c(-c5ccccc5)cccc3n4-c3ccccc3)cc21]

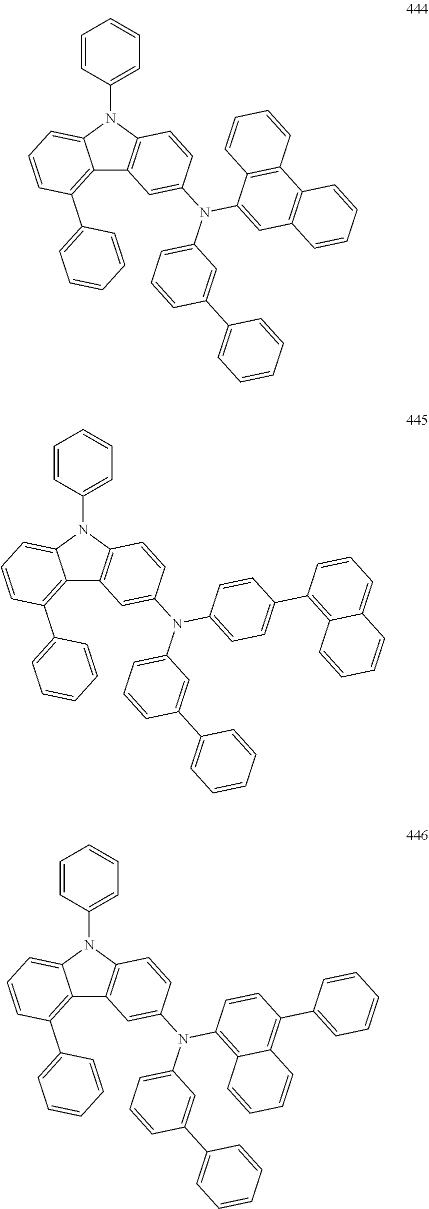 [SMILES: c1ccc(-c2cccc(N(c3ccc4c(c3)c3c(-c5ccccc5)cccc3n4-c3ccccc3)c3cc4ccccc4c4ccccc34)c2)cc1 ; c1ccc(-c2cccc(N(c3ccc(-c4cccc5ccccc45)cc3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3ccc(-c4ccccc4)c4ccccc34)c2)cc1]

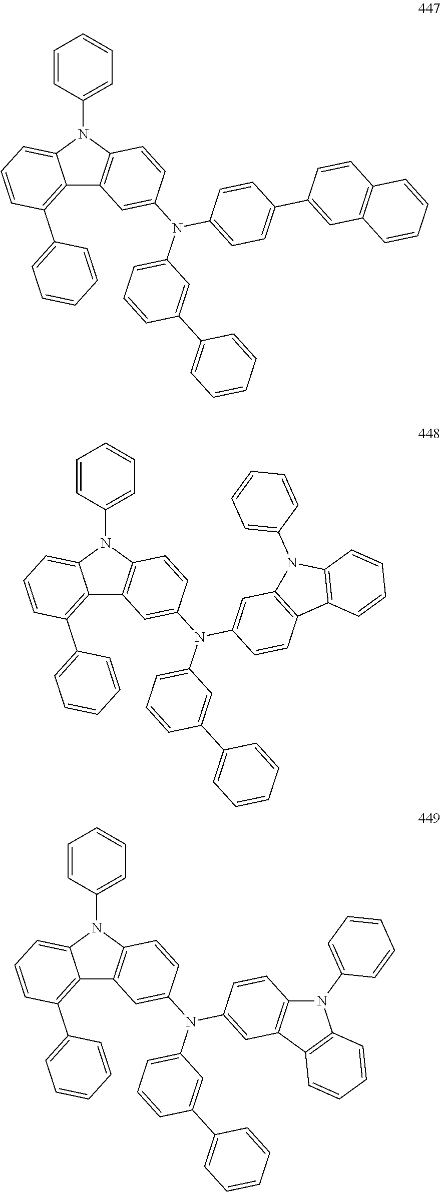 [SMILES: c1ccc(-c2cccc(N(c3ccc(-c4ccc5ccccc5c4)cc3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3ccc4c5ccccc5n(-c5ccccc5)c4c3)c2)cc1 ; c1ccc(-c2cccc(N(c3ccc4c(c3)c3ccccc3n4-c3ccccc3)c3ccc4c(c3)c3c(-c5ccccc5)cccc3n4-c3ccccc3)c2)cc1]

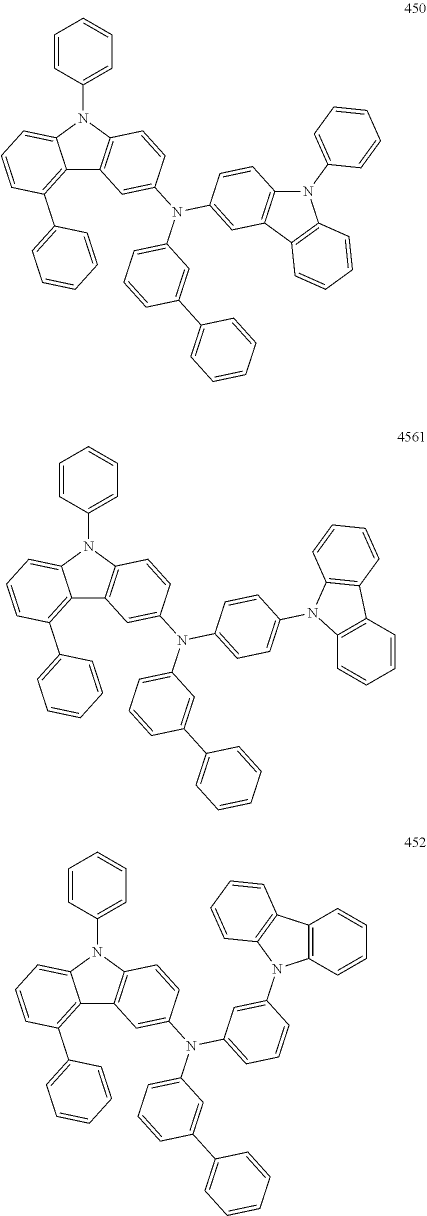 [SMILES: c1ccc(-c2cccc(N(c3ccc4c(c3)c3ccccc3n4-c3ccccc3)c3ccc4c(c3)c3c(-c5ccccc5)cccc3n4-c3ccccc3)c2)cc1 ; c1ccc(-c2cccc(N(c3ccc(-n4c5ccccc5c5ccccc54)cc3)c3ccc4c(c3)c3c(-c5ccccc5)cccc3n4-c3ccccc3)c2)cc1 ; c1ccc(-c2cccc(N(c3cccc(-n4c5ccccc5c5ccccc54)c3)c3ccc4c(c3)c3c(-c5ccccc5)cccc3n4-c3ccccc3)c2)cc1]

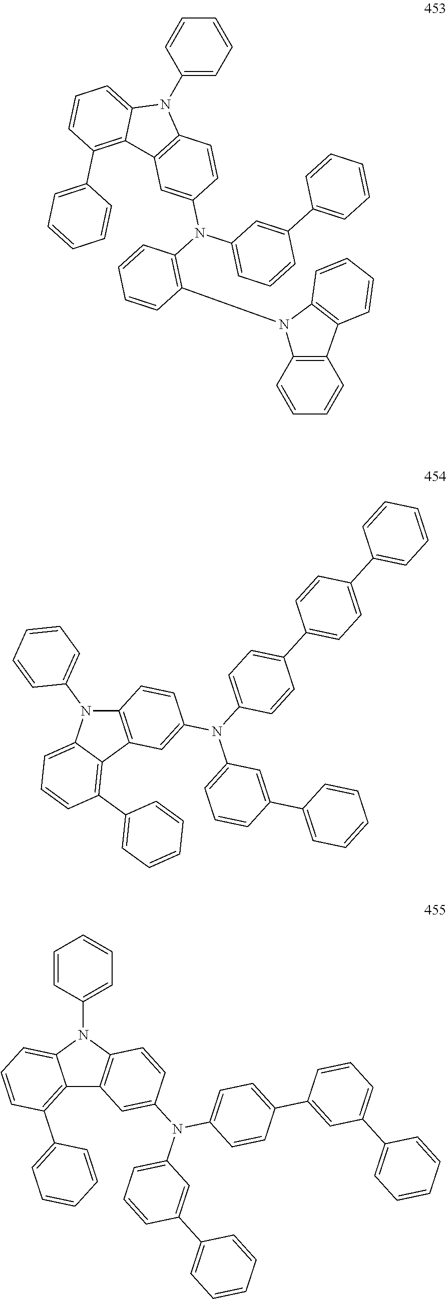 [SMILES: c1ccc(-c2cccc(N(c3ccc4c(c3)c3c(-c5ccccc5)cccc3n4-c3ccccc3)c3ccccc3-n3c4ccccc4c4ccccc43)c2)cc1 ; c1ccc(-c2ccc(-c3ccc(N(c4cccc(-c5ccccc5)c4)c4ccc5c(c4)c4c(-c6ccccc6)cccc4n5-c4ccccc4)cc3)cc2)cc1 ; c1ccc(-c2cccc(-c3ccc(N(c4cccc(-c5ccccc5)c4)c4ccc5c(c4)c4c(-c6ccccc6)cccc4n5-c4ccccc4)cc3)c2)cc1]

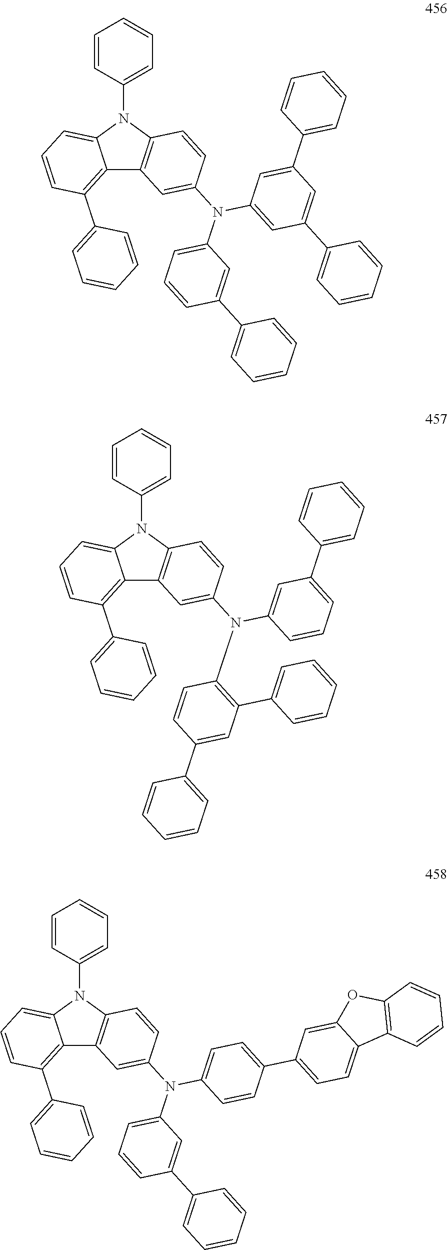 [SMILES: c1ccc(-c2cccc(N(c3cc(-c4ccccc4)cc(-c4ccccc4)c3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3ccc(-c4ccccc4)cc3-c3ccccc3)c2)cc1 ; c1ccc(-c2cccc(N(c3ccc(-c4ccc5c(c4)oc4ccccc45)cc3)c3ccc4c(c3)c3c(-c5ccccc5)cccc3n4-c3ccccc3)c2)cc1]

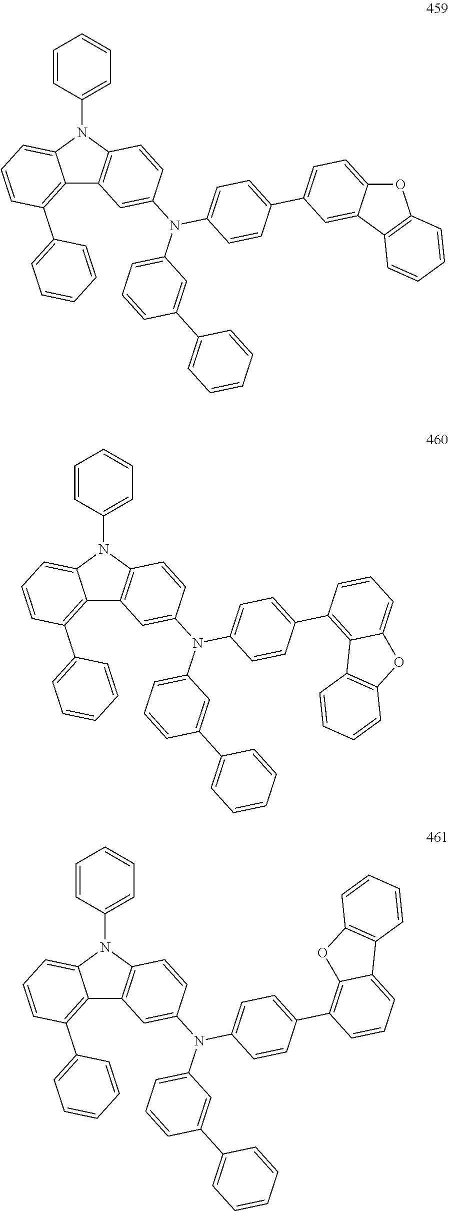 [SMILES: c1ccc(-c2cccc(N(c3ccc(-c4ccc5oc6ccccc6c5c4)cc3)c3ccc4c(c3)c3c(-c5ccccc5)cccc3n4-c3ccccc3)c2)cc1 ; c1ccc(-c2cccc(N(c3ccc(-c4cccc5oc6ccccc6c45)cc3)c3ccc4c(c3)c3c(-c5ccccc5)cccc3n4-c3ccccc3)c2)cc1 ; c1ccc(-c2cccc(N(c3ccc(-c4cccc5c4oc4ccccc45)cc3)c3ccc4c(c3)c3c(-c5ccccc5)cccc3n4-c3ccccc3)c2)cc1]

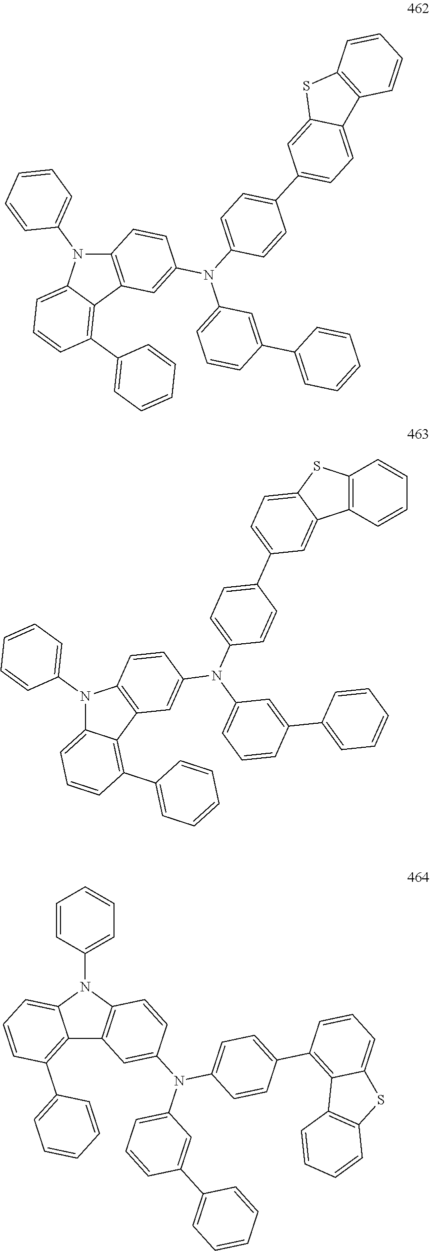 [SMILES: c1ccc(-c2cccc(N(c3ccc(-c4ccc5c(c4)sc4ccccc45)cc3)c3ccc4c(c3)c3c(-c5ccccc5)cccc3n4-c3ccccc3)c2)cc1 ; c1ccc(-c2cccc(N(c3ccc(-c4ccc5sc6ccccc6c5c4)cc3)c3ccc4c(c3)c3c(-c5ccccc5)cccc3n4-c3ccccc3)c2)cc1 ; c1ccc(-c2cccc(N(c3ccc(-c4cccc5sc6ccccc6c45)cc3)c3ccc4c(c3)c3c(-c5ccccc5)cccc3n4-c3ccccc3)c2)cc1]

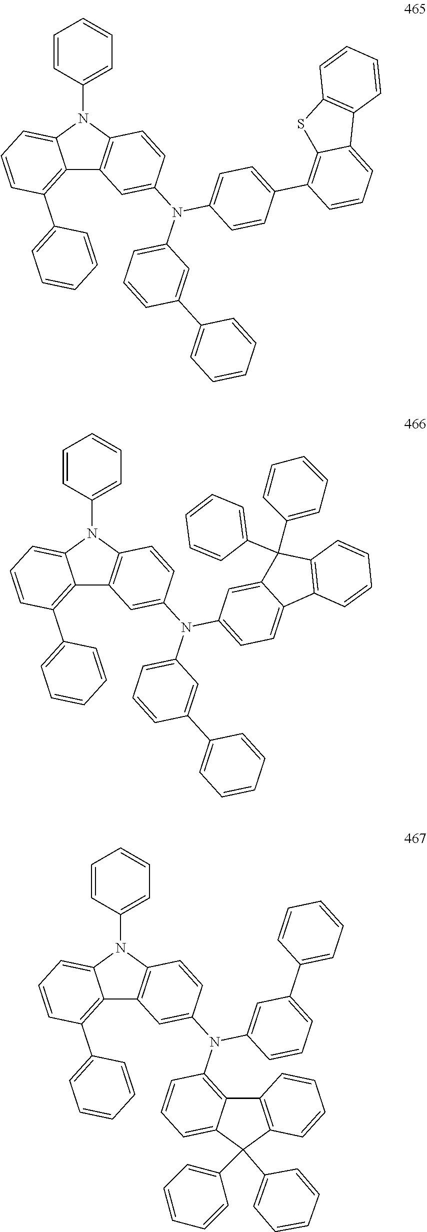 [SMILES: c1ccc(-c2cccc(N(c3ccc(-c4cccc5c4sc4ccccc45)cc3)c3ccc4c(c3)c3c(-c5ccccc5)cccc3n4-c3ccccc3)c2)cc1 ; c1ccc(-c2cccc(N(c3ccc4c(c3)C(c3ccccc3)(c3ccccc3)c3ccccc3-4)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3cccc4c3-c3ccccc3C4(c3ccccc3)c3ccccc3)c2)cc1]

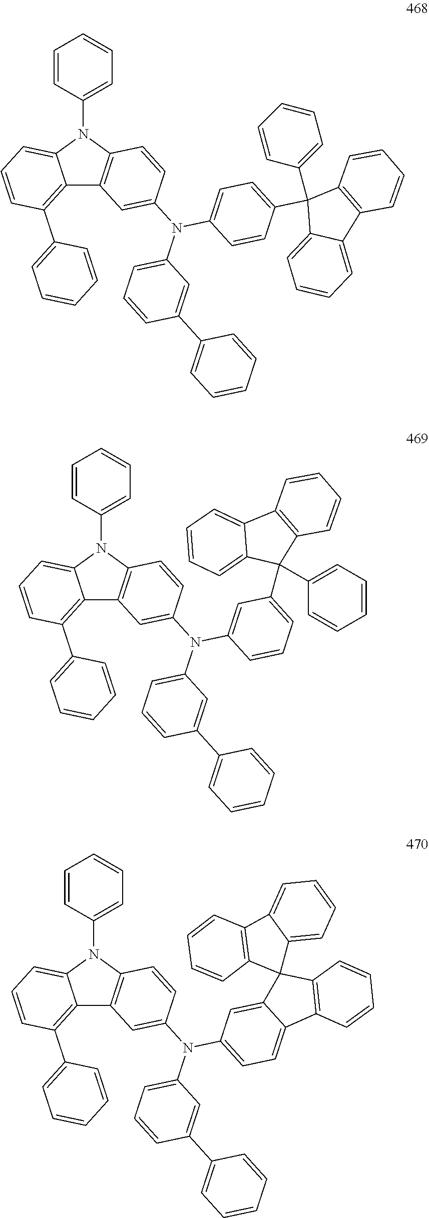 [SMILES: c1ccc(-c2cccc(N(c3ccc(C4(c5ccccc5)c5ccccc5-c5ccccc54)cc3)c3ccc4c(c3)c3c(-c5ccccc5)cccc3n4-c3ccccc3)c2)cc1 ; c1ccc(-c2cccc(N(c3cccc(C4(c5ccccc5)c5ccccc5-c5ccccc54)c3)c3ccc4c(c3)c3c(-c5ccccc5)cccc3n4-c3ccccc3)c2)cc1 ; c1ccc(-c2cccc(N(c3ccc4c(c3)C3(c5ccccc5-c5ccccc53)c3ccccc3-4)c3ccc4c(c3)c3c(-c5ccccc5)cccc3n4-c3ccccc3)c2)cc1]

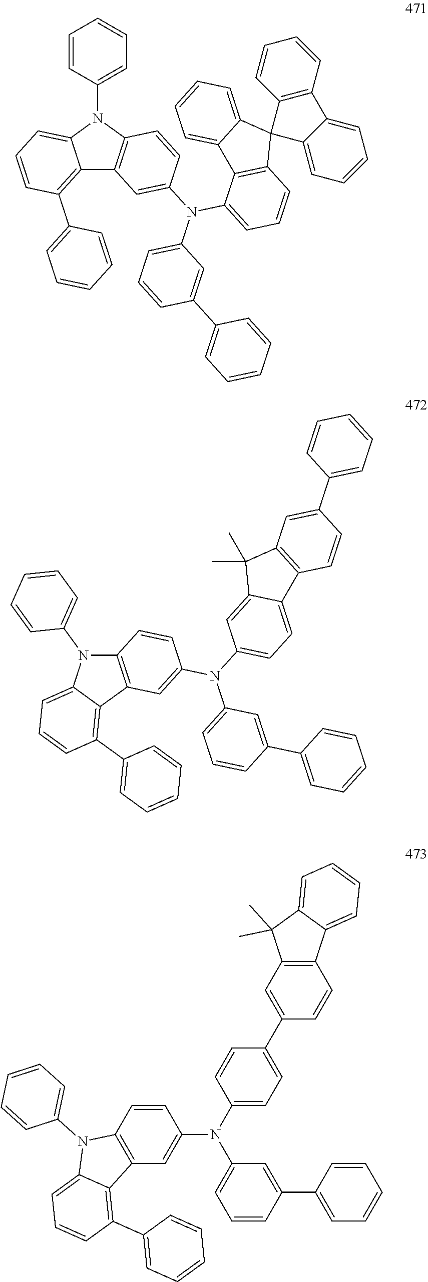 [SMILES: c1ccc(-c2cccc(N(c3ccc4c(c3)c3c(-c5ccccc5)cccc3n4-c3ccccc3)c3cccc4c3-c3ccccc3C43c4ccccc4-c4ccccc43)c2)cc1 ; CC1(C)c2cc(-c3ccccc3)ccc2-c2ccc(N(c3cccc(-c4ccccc4)c3)c3ccc4c(c3)c3c(-c5ccccc5)cccc3n4-c3ccccc3)cc21 ; CC1(C)c2ccccc2-c2ccc(-c3ccc(N(c4cccc(-c5ccccc5)c4)c4ccc5c(c4)c4c(-c6ccccc6)cccc4n5-c4ccccc4)cc3)cc21]

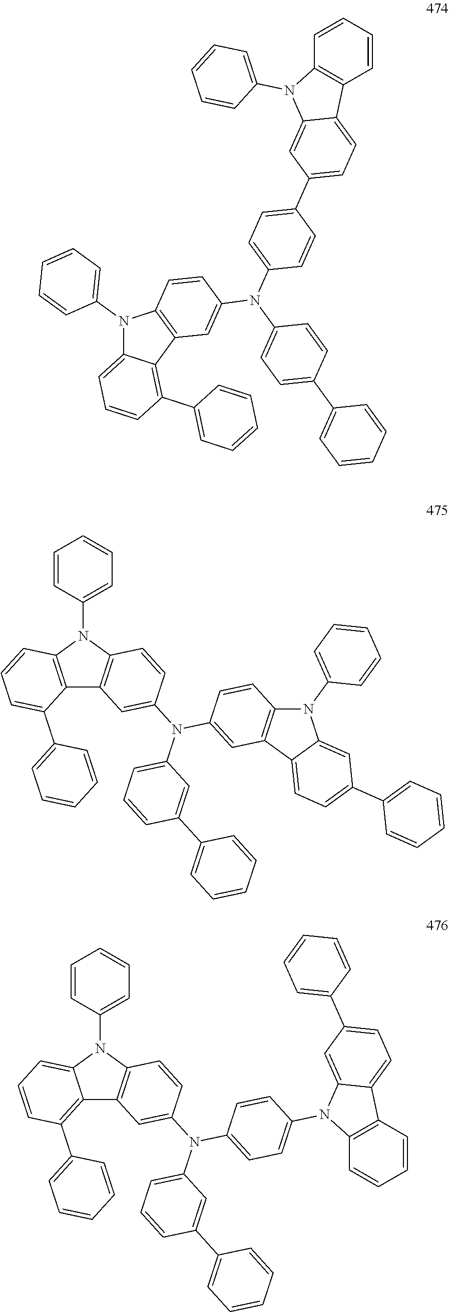 [SMILES: c1ccc(-c2ccc(N(c3ccc(-c4ccc5c6ccccc6n(-c6ccccc6)c5c4)cc3)c3ccc4c(c3)c3c(-c5ccccc5)cccc3n4-c3ccccc3)cc2)cc1 ; c1ccc(-c2cccc(N(c3ccc4c(c3)c3ccc(-c5ccccc5)cc3n4-c3ccccc3)c3ccc4c(c3)c3c(-c5ccccc5)cccc3n4-c3ccccc3)c2)cc1 ; c1ccc(-c2cccc(N(c3ccc(-n4c5ccccc5c5ccc(-c6ccccc6)cc54)cc3)c3ccc4c(c3)c3c(-c5ccccc5)cccc3n4-c3ccccc3)c2)cc1]

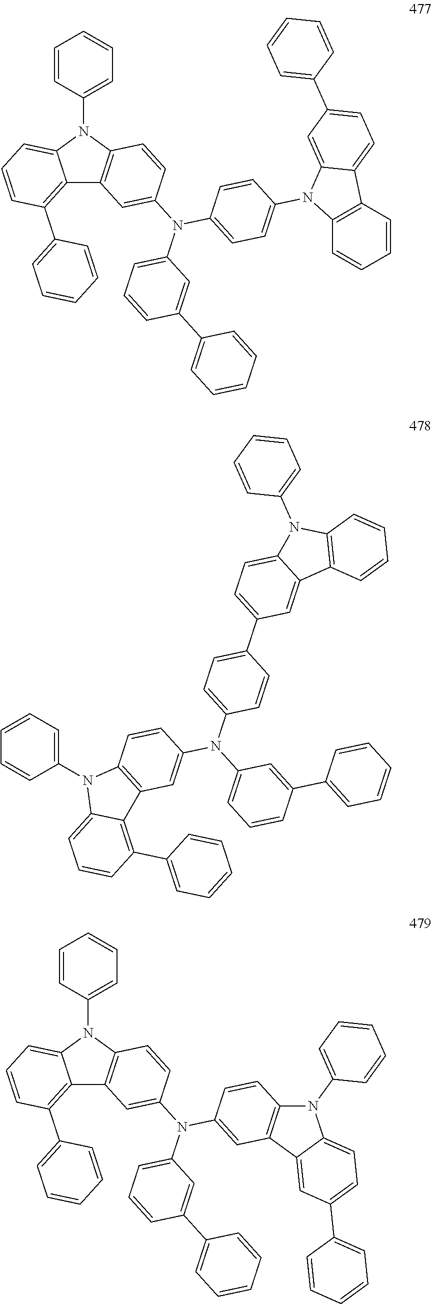 [SMILES: c1ccc(-c2cccc(N(c3ccc(-n4c5ccccc5c5ccc(-c6ccccc6)cc54)cc3)c3ccc4c(c3)c3c(-c5ccccc5)cccc3n4-c3ccccc3)c2)cc1 ; c1ccc(-c2cccc(N(c3ccc(-c4ccc5c(c4)c4ccccc4n5-c4ccccc4)cc3)c3ccc4c(c3)c3c(-c5ccccc5)cccc3n4-c3ccccc3)c2)cc1 ; c1ccc(-c2cccc(N(c3ccc4c(c3)c3cc(-c5ccccc5)ccc3n4-c3ccccc3)c3ccc4c(c3)c3c(-c5ccccc5)cccc3n4-c3ccccc3)c2)cc1]

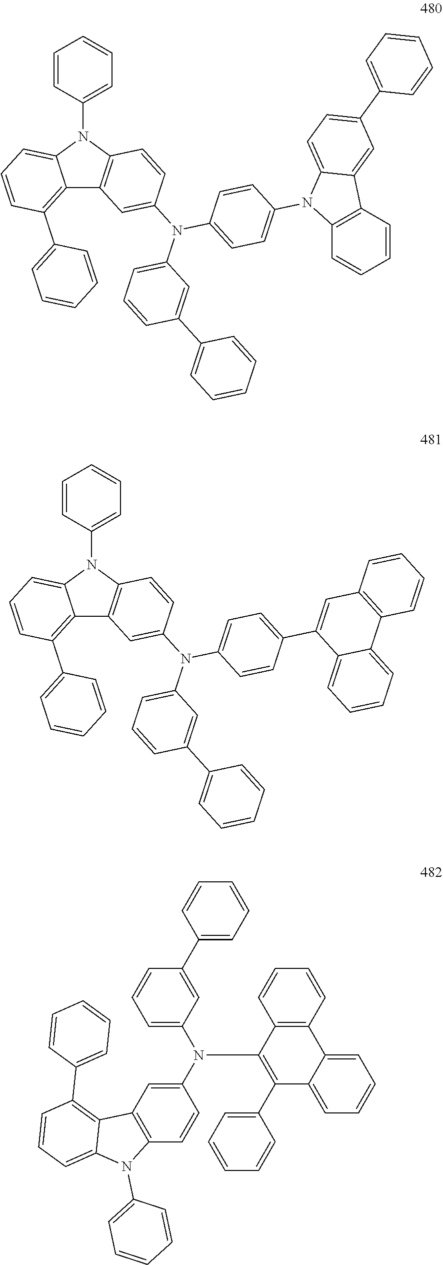 [SMILES: c1ccc(-c2cccc(N(c3ccc(-n4c5ccccc5c5cc(-c6ccccc6)ccc54)cc3)c3ccc4c(c3)c3c(-c5ccccc5)cccc3n4-c3ccccc3)c2)cc1 ; c1ccc(-c2cccc(N(c3ccc(-c4cc5ccccc5c5ccccc45)cc3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3c(-c4ccccc4)c4ccccc4c4ccccc34)c2)cc1]

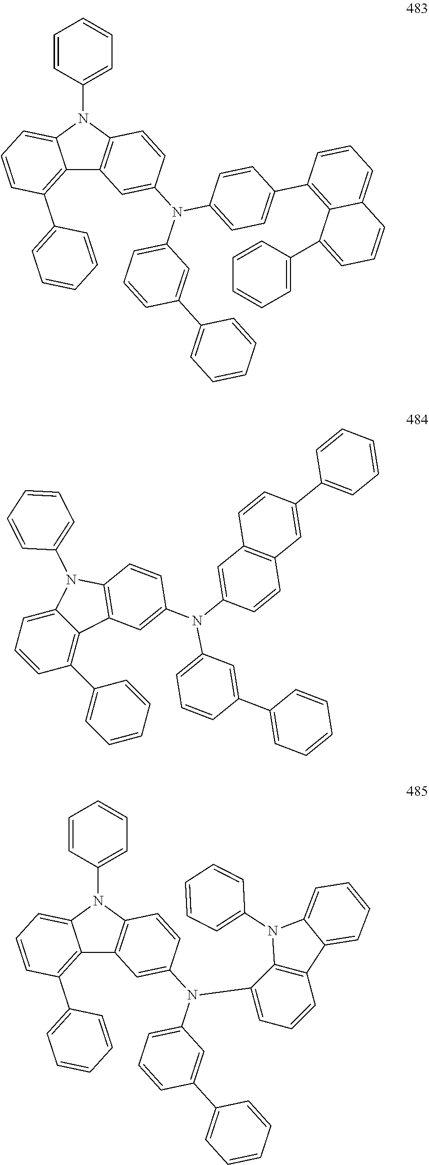 [SMILES: c1ccc(-c2cccc(N(c3ccc(-c4cccc5cccc(-c6ccccc6)c45)cc3)c3ccc4c(c3)c3c(-c5ccccc5)cccc3n4-c3ccccc3)c2)cc1 ; c1ccc(-c2cccc(N(c3ccc4cc(-c5ccccc5)ccc4c3)c3ccc4c(c3)c3c(-c5ccccc5)cccc3n4-c3ccccc3)c2)cc1 ; c1ccc(-c2cccc(N(c3ccc4c(c3)c3c(-c5ccccc5)cccc3n4-c3ccccc3)c3cccc4c5ccccc5n(-c5ccccc5)c34)c2)cc1]

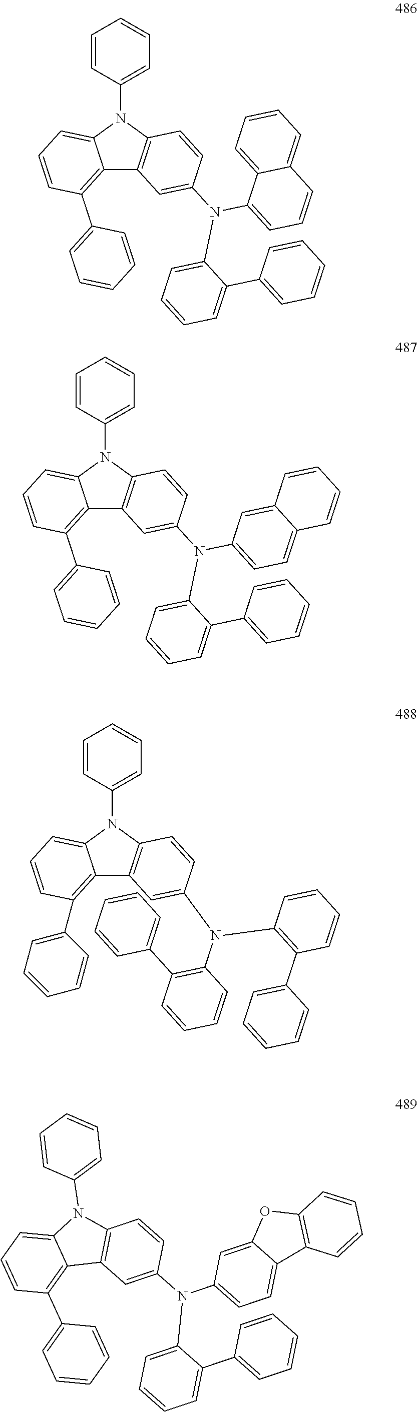 [SMILES: c1ccc(-c2ccccc2N(c2ccc3c(c2)c2c(-c4ccccc4)cccc2n3-c2ccccc2)c2cccc3ccccc23)cc1 ; c1ccc(-c2ccccc2N(c2ccc3ccccc3c2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2ccccc2-c2ccccc2)cc1 ; c1ccc(-c2ccccc2N(c2ccc3c(c2)oc2ccccc23)c2ccc3c(c2)c2c(-c4ccccc4)cccc2n3-c2ccccc2)cc1]

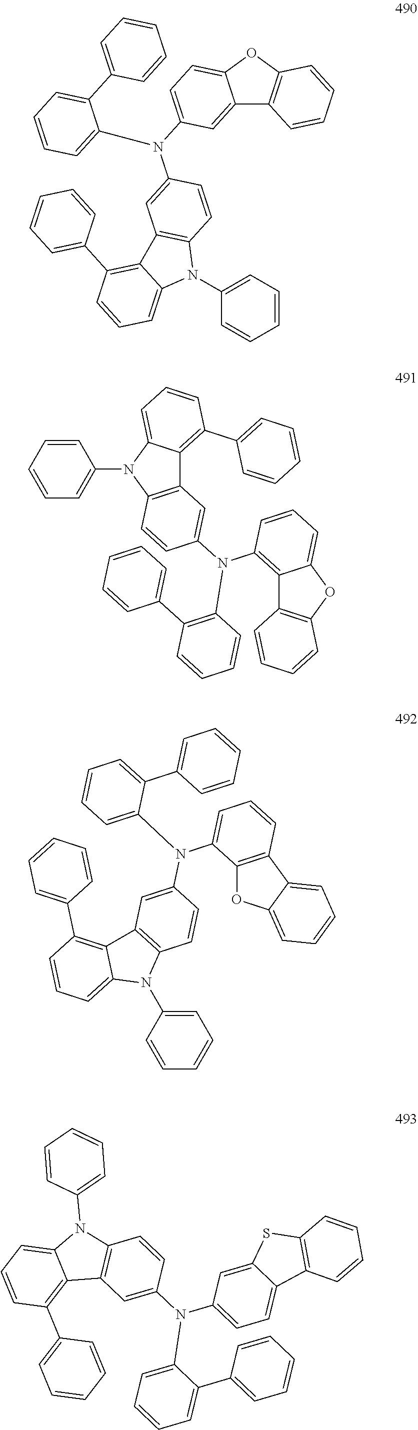 [SMILES: c1ccc(-c2ccccc2N(c2ccc3oc4ccccc4c3c2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2cccc3oc4ccccc4c23)cc1 ; c1ccc(-c2ccccc2N(c2ccc3c(c2)c2c(-c4ccccc4)cccc2n3-c2ccccc2)c2cccc3c2oc2ccccc23)cc1 ; c1ccc(-c2ccccc2N(c2ccc3c(c2)sc2ccccc23)c2ccc3c(c2)c2c(-c4ccccc4)cccc2n3-c2ccccc2)cc1]

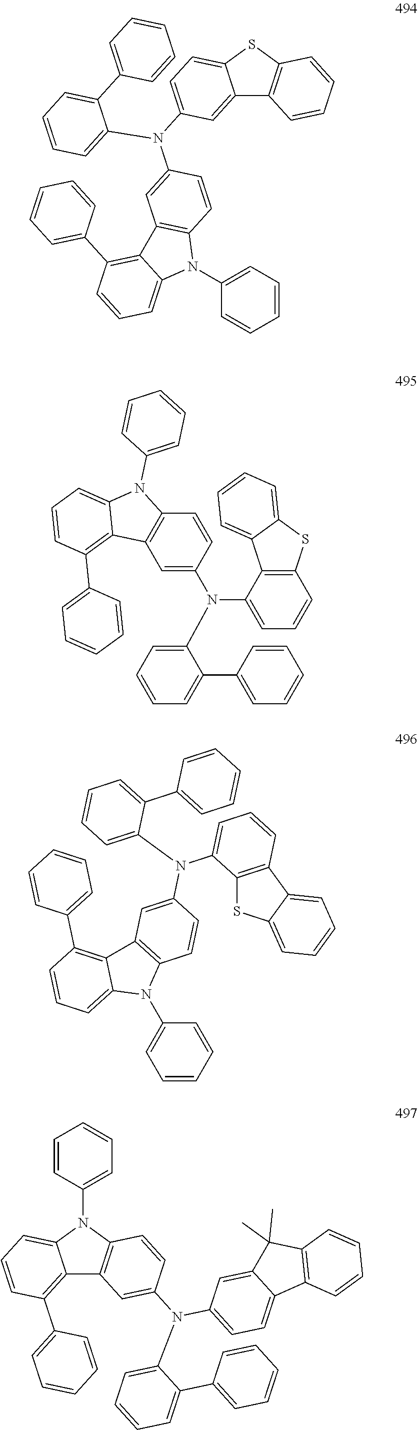 [SMILES: c1ccc(-c2ccccc2N(c2ccc3sc4ccccc4c3c2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2cccc3sc4ccccc4c23)cc1 ; c1ccc(-c2ccccc2N(c2ccc3c(c2)c2c(-c4ccccc4)cccc2n3-c2ccccc2)c2cccc3c2sc2ccccc23)cc1 ; CC1(C)c2ccccc2-c2ccc(N(c3ccc4c(c3)c3c(-c5ccccc5)cccc3n4-c3ccccc3)c3ccccc3-c3ccccc3)cc21]

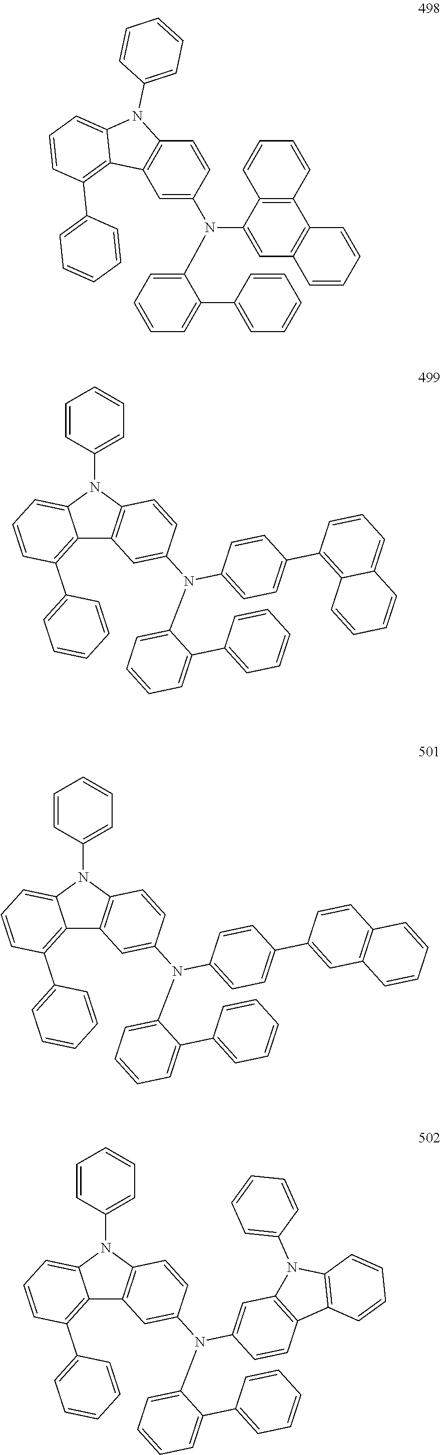 [SMILES: c1ccc(-c2ccccc2N(c2ccc3c(c2)c2c(-c4ccccc4)cccc2n3-c2ccccc2)c2cc3ccccc3c3ccccc23)cc1 ; c1ccc(-c2ccccc2N(c2ccc(-c3cccc4ccccc34)cc2)c2ccc3c(c2)c2c(-c4ccccc4)cccc2n3-c2ccccc2)cc1 ; c1ccc(-c2ccccc2N(c2ccc(-c3ccc4ccccc4c3)cc2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2ccc3c4ccccc4n(-c4ccccc4)c3c2)cc1]

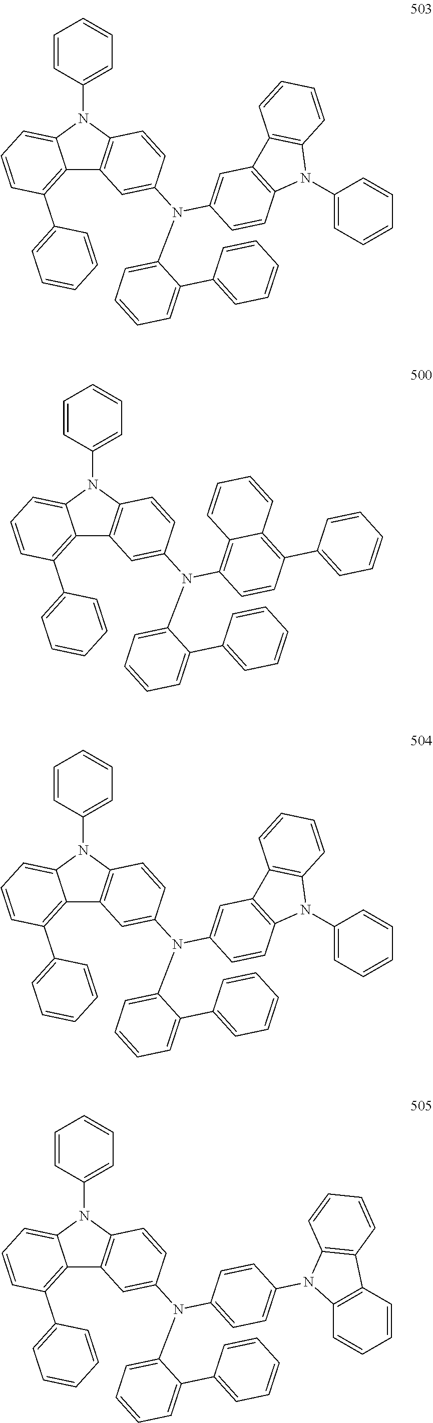 [SMILES: c1ccc(-c2ccccc2N(c2ccc3c(c2)c2ccccc2n3-c2ccccc2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2ccc(-c3ccccc3)c3ccccc23)cc1 ; c1ccc(-c2ccccc2N(c2ccc3c(c2)c2ccccc2n3-c2ccccc2)c2ccc3c(c2)c2c(-c4ccccc4)cccc2n3-c2ccccc2)cc1 ; c1ccc(-c2ccccc2N(c2ccc(-n3c4ccccc4c4ccccc43)cc2)c2ccc3c(c2)c2c(-c4ccccc4)cccc2n3-c2ccccc2)cc1]

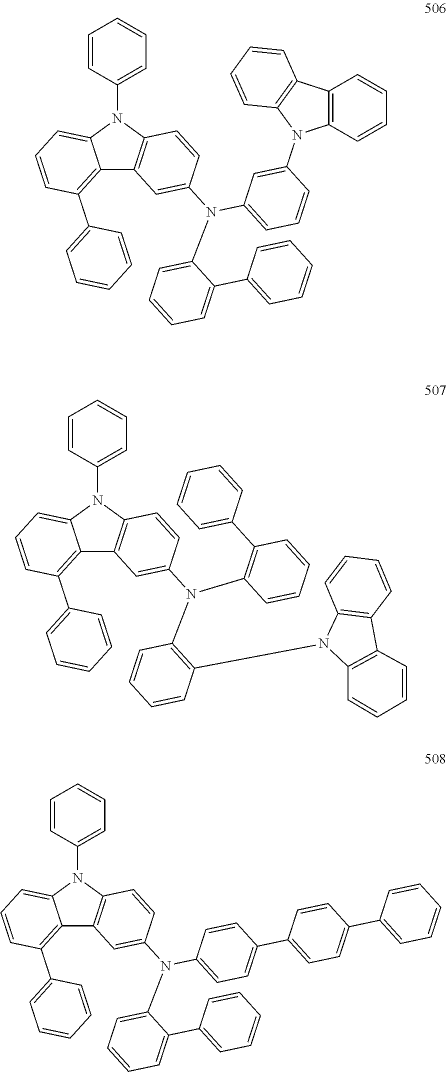 [SMILES: c1ccc(-c2ccccc2N(c2cccc(-n3c4ccccc4c4ccccc43)c2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2ccccc2-n2c3ccccc3c3ccccc32)cc1 ; c1ccc(-c2ccc(-c3ccc(N(c4ccc5c(c4)c4c(-c6ccccc6)cccc4n5-c4ccccc4)c4ccccc4-c4ccccc4)cc3)cc2)cc1]

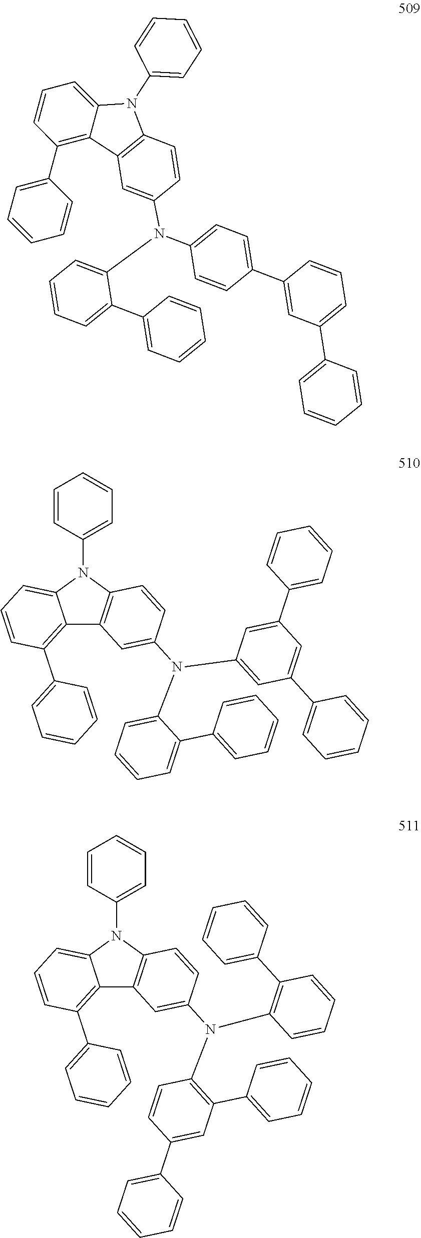 [SMILES: c1ccc(-c2cccc(-c3ccc(N(c4ccc5c(c4)c4c(-c6ccccc6)cccc4n5-c4ccccc4)c4ccccc4-c4ccccc4)cc3)c2)cc1 ; c1ccc(-c2cc(-c3ccccc3)cc(N(c3ccc4c(c3)c3c(-c5ccccc5)cccc3n4-c3ccccc3)c3ccccc3-c3ccccc3)c2)cc1 ; c1ccc(-c2ccc(N(c3ccc4c(c3)c3c(-c5ccccc5)cccc3n4-c3ccccc3)c3ccccc3-c3ccccc3)c(-c3ccccc3)c2)cc1]

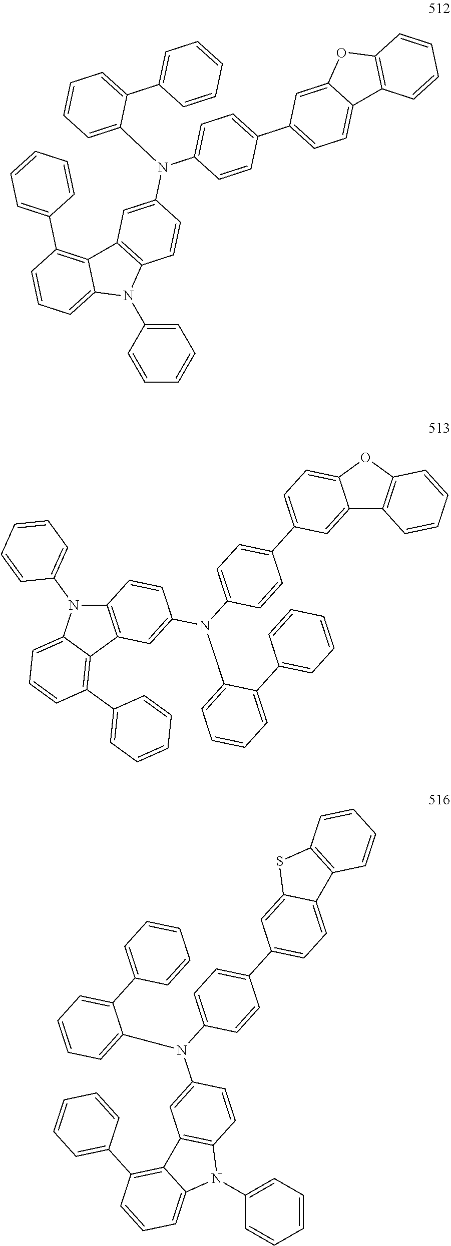 [SMILES: c1ccc(-c2ccccc2N(c2ccc(-c3ccc4c(c3)oc3ccccc34)cc2)c2ccc3c(c2)c2c(-c4ccccc4)cccc2n3-c2ccccc2)cc1 ; c1ccc(-c2ccccc2N(c2ccc(-c3ccc4oc5ccccc5c4c3)cc2)c2ccc3c(c2)c2c(-c4ccccc4)cccc2n3-c2ccccc2)cc1 ; c1ccc(-c2ccccc2N(c2ccc(-c3ccc4c(c3)sc3ccccc34)cc2)c2ccc3c(c2)c2c(-c4ccccc4)cccc2n3-c2ccccc2)cc1]

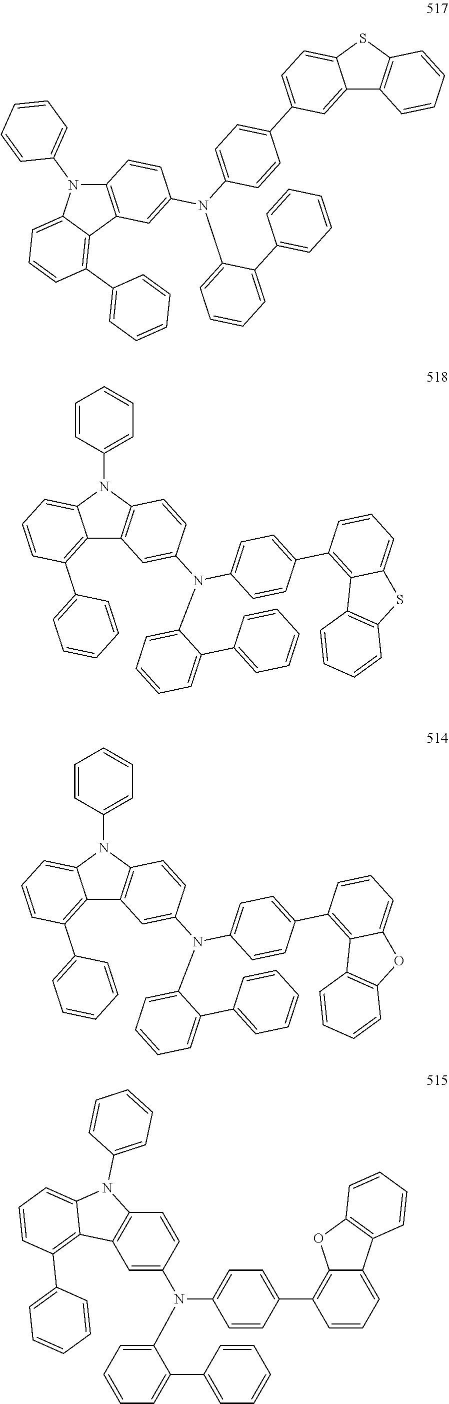

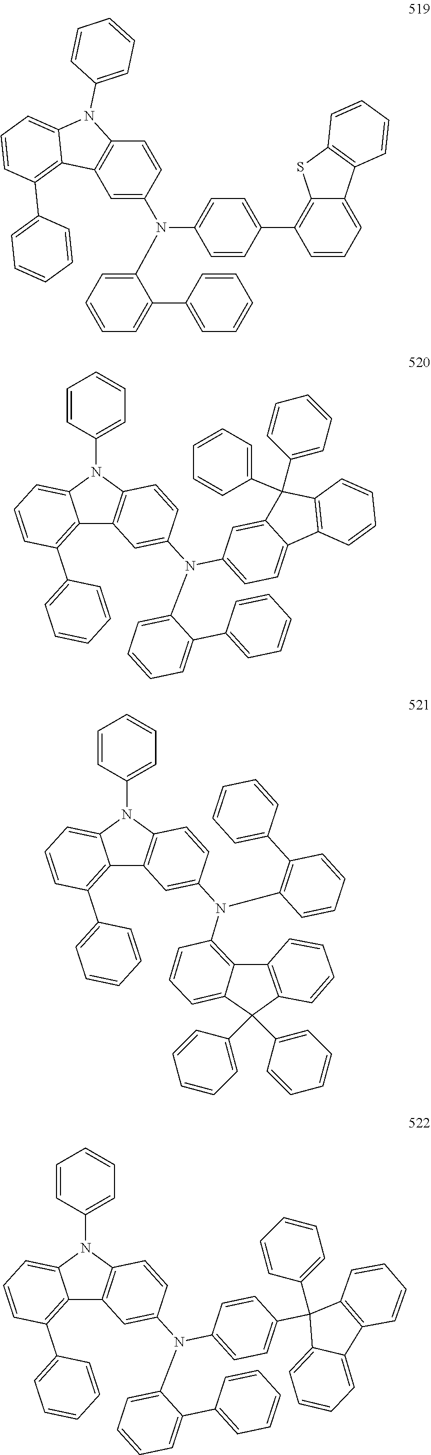

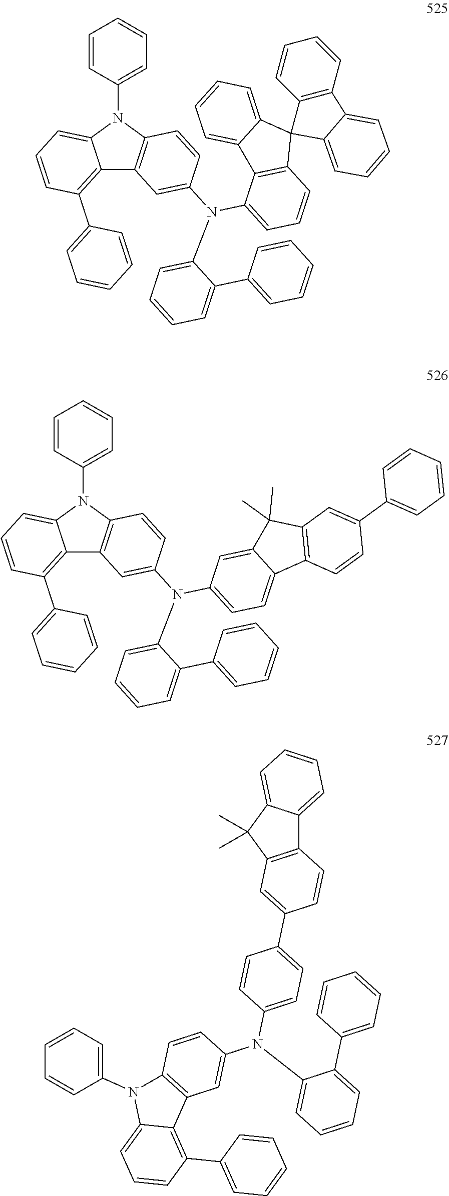 [SMILES: c1ccc(-c2ccccc2N(c2ccc3c(c2)c2c(-c4ccccc4)cccc2n3-c2ccccc2)c2cccc3c2-c2ccccc2C32c3ccccc3-c3ccccc32)cc1 ; CC1(C)c2cc(-c3ccccc3)ccc2-c2ccc(N(c3ccc4c(c3)c3c(-c5ccccc5)cccc3n4-c3ccccc3)c3ccccc3-c3ccccc3)cc21 ; CC1(C)c2ccccc2-c2ccc(-c3ccc(N(c4ccc5c(c4)c4c(-c6ccccc6)cccc4n5-c4ccccc4)c4ccccc4-c4ccccc4)cc3)cc21]

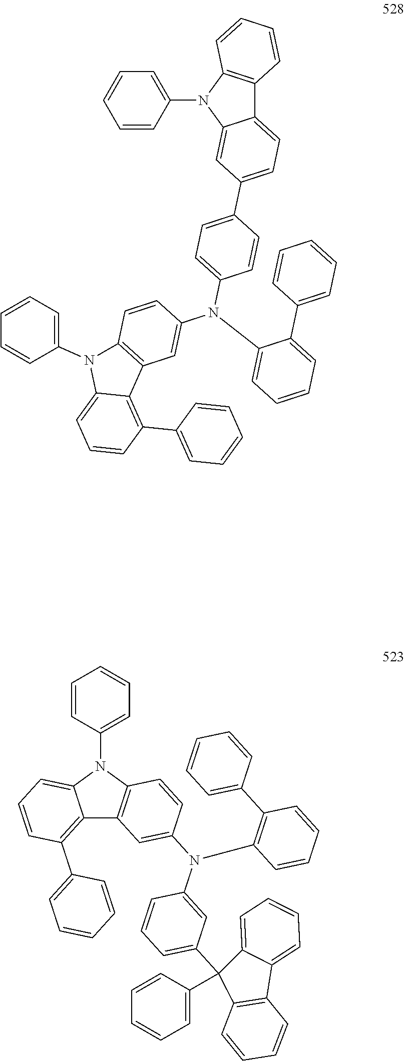 [SMILES: c1ccc(-c2ccccc2N(c2ccc(-c3ccc4c5ccccc5n(-c5ccccc5)c4c3)cc2)c2ccc3c(c2)c2c(-c4ccccc4)cccc2n3-c2ccccc2)cc1 ; c1ccc(-c2ccccc2N(c2cccc(C3(c4ccccc4)c4ccccc4-c4ccccc43)c2)c2ccc3c(c2)c2c(-c4ccccc4)cccc2n3-c2ccccc2)cc1]

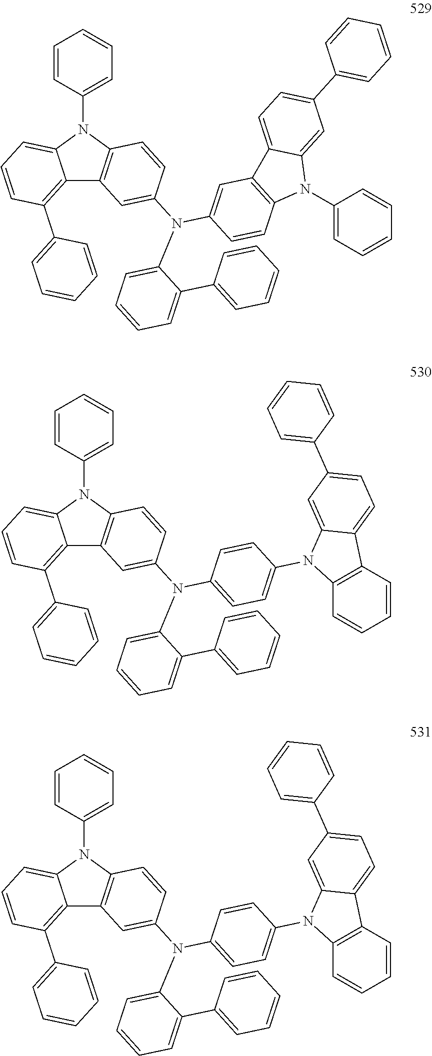 [SMILES: c1ccc(-c2ccc3c4cc(N(c5ccc6c(c5)c5c(-c7ccccc7)cccc5n6-c5ccccc5)c5ccccc5-c5ccccc5)ccc4n(-c4ccccc4)c3c2)cc1 ; c1ccc(-c2ccc3c4ccccc4n(-c4ccc(N(c5ccc6c(c5)c5c(-c7ccccc7)cccc5n6-c5ccccc5)c5ccccc5-c5ccccc5)cc4)c3c2)cc1 ; c1ccc(-c2ccc3c4ccccc4n(-c4ccc(N(c5ccc6c(c5)c5c(-c7ccccc7)cccc5n6-c5ccccc5)c5ccccc5-c5ccccc5)cc4)c3c2)cc1]

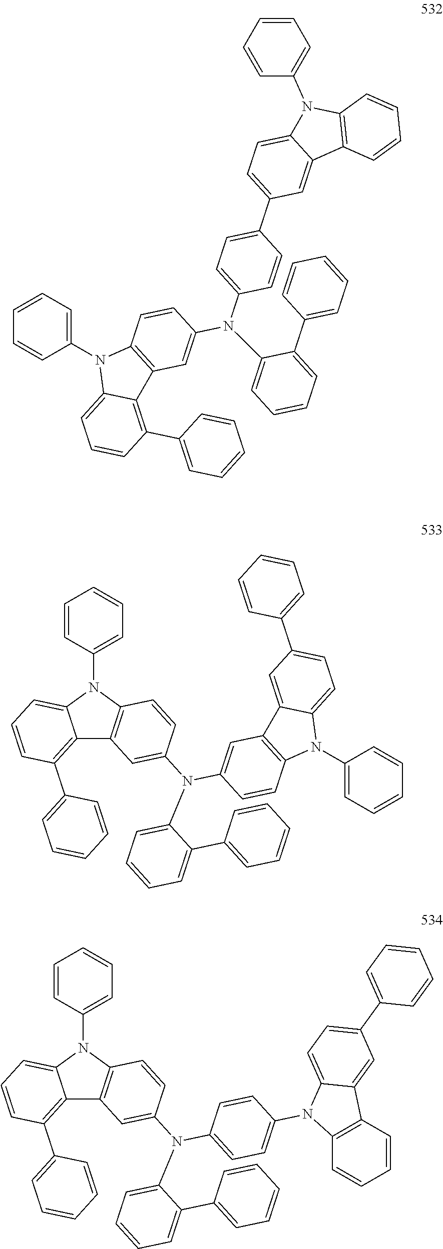 [SMILES: c1ccc(-c2ccccc2N(c2ccc(-c3ccc4c(c3)c3ccccc3n4-c3ccccc3)cc2)c2ccc3c(c2)c2c(-c4ccccc4)cccc2n3-c2ccccc2)cc1 ; c1ccc(-c2ccc3c(c2)c2cc(N(c4ccc5c(c4)c4c(-c6ccccc6)cccc4n5-c4ccccc4)c4ccccc4-c4ccccc4)ccc2n3-c2ccccc2)cc1 ; c1ccc(-c2ccc3c(c2)c2ccccc2n3-c2ccc(N(c3ccc4c(c3)c3c(-c5ccccc5)cccc3n4-c3ccccc3)c3ccccc3-c3ccccc3)cc2)cc1]

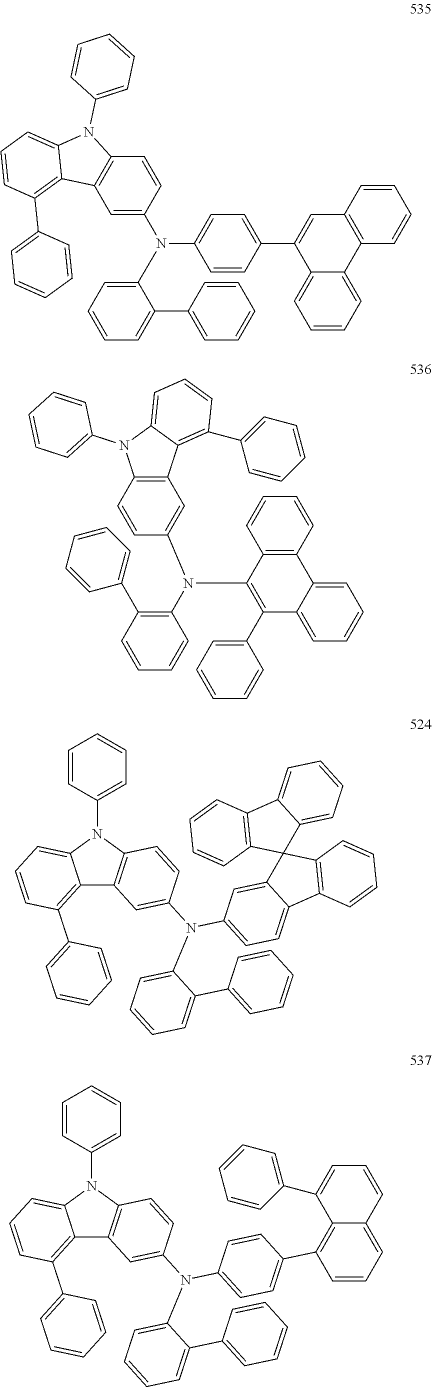

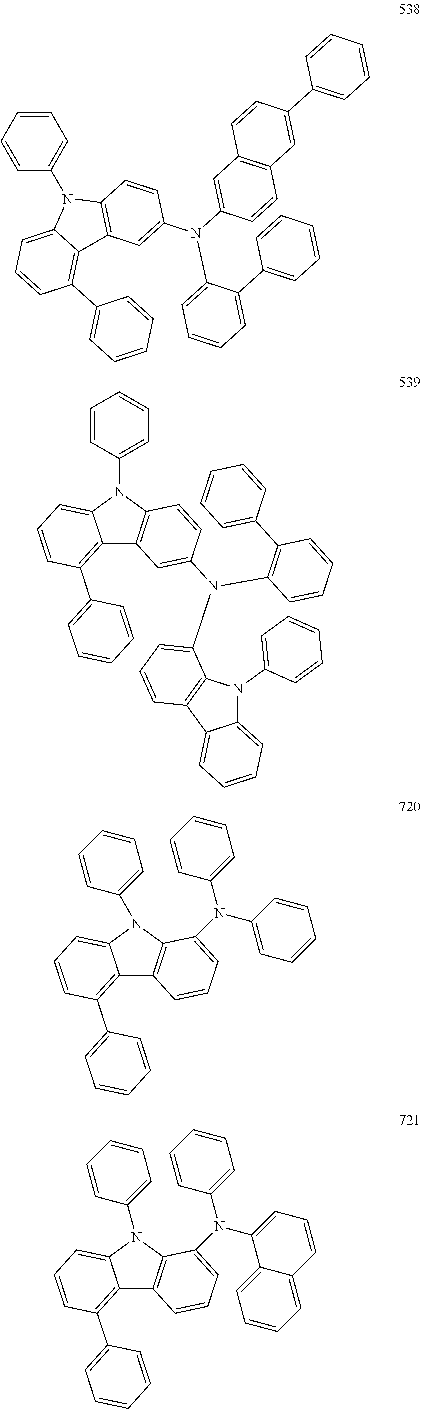

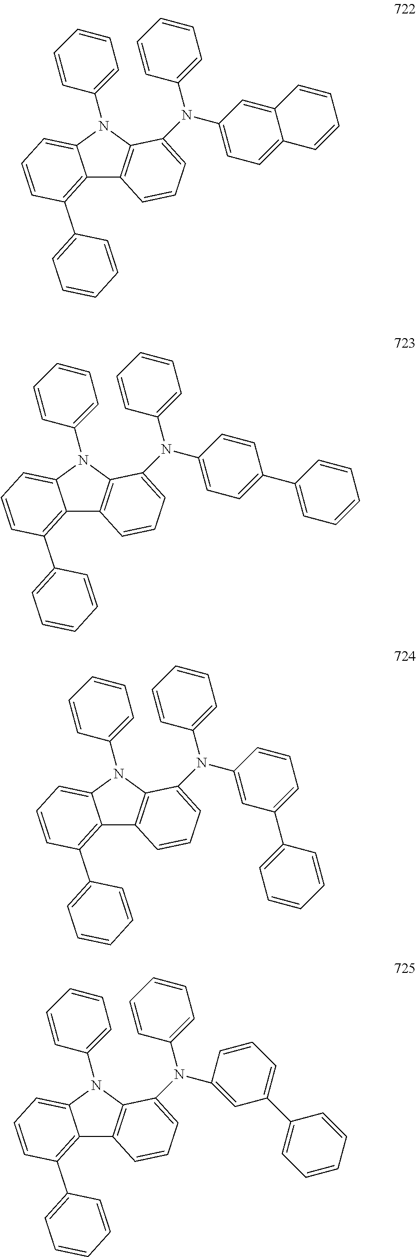 [SMILES: c1ccc(-c2cccc3c2c2cccc(N(c4ccccc4)c4ccc5ccccc5c4)c2n3-c2ccccc2)cc1 ; c1ccc(-c2ccc(N(c3ccccc3)c3cccc4c5c(-c6ccccc6)cccc5n(-c5ccccc5)c34)cc2)cc1 ; c1ccc(-c2cccc(N(c3ccccc3)c3cccc4c5c(-c6ccccc6)cccc5n(-c5ccccc5)c34)c2)cc1 ; c1ccc(-c2cccc(N(c3ccccc3)c3cccc4c5c(-c6ccccc6)cccc5n(-c5ccccc5)c34)c2)cc1]

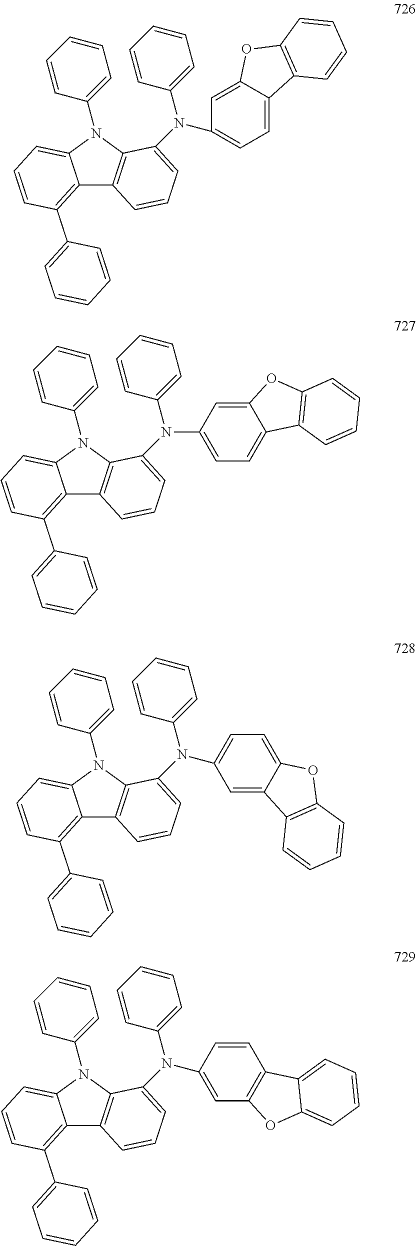 [SMILES: c1ccc(-c2cccc3c2c2cccc(N(c4ccccc4)c4ccc5c(c4)oc4ccccc45)c2n3-c2ccccc2)cc1 ; c1ccc(-c2cccc3c2c2cccc(N(c4ccccc4)c4ccc5c(c4)oc4ccccc45)c2n3-c2ccccc2)cc1 ; c1ccc(-c2cccc3c2c2cccc(N(c4ccccc4)c4ccc5oc6ccccc6c5c4)c2n3-c2ccccc2)cc1 ; c1ccc(-c2cccc3c2c2cccc(N(c4ccccc4)c4ccc5c(c4)oc4ccccc45)c2n3-c2ccccc2)cc1]

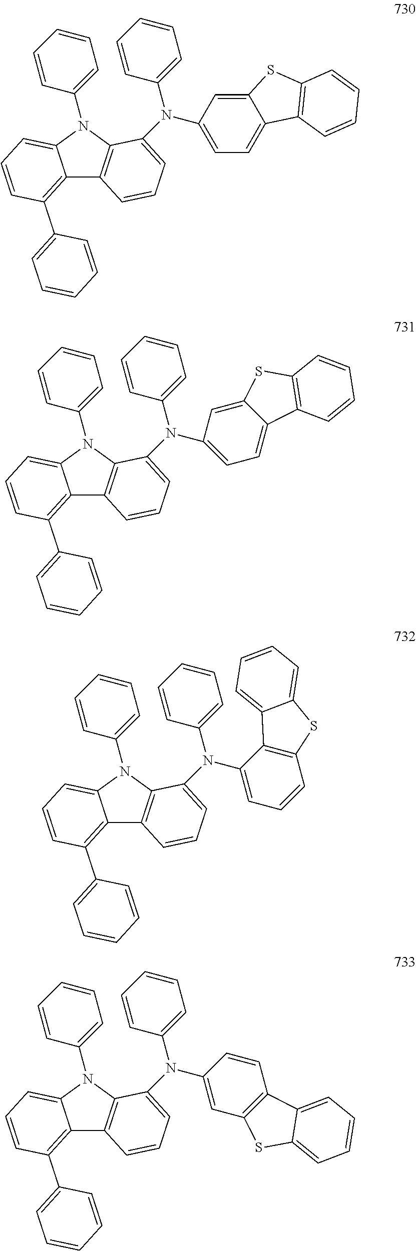

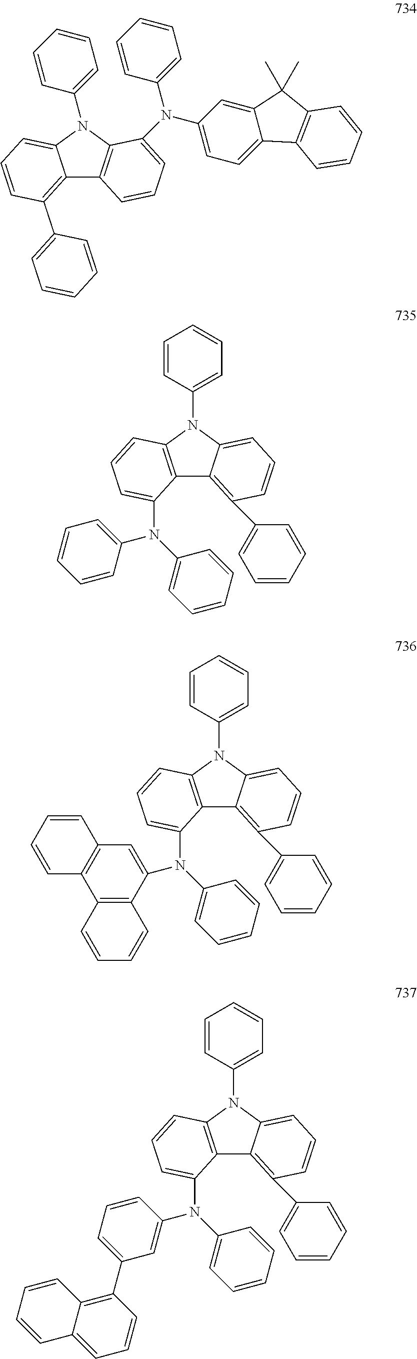 [SMILES: CC1(C)c2ccccc2-c2ccc(N(c3ccccc3)c3cccc4c5c(-c6ccccc6)cccc5n(-c5ccccc5)c34)cc21 ; c1ccc(-c2cccc3c2c2c(N(c4ccccc4)c4ccccc4)cccc2n3-c2ccccc2)cc1 ; c1ccc(-c2cccc3c2c2c(N(c4ccccc4)c4cc5ccccc5c5ccccc45)cccc2n3-c2ccccc2)cc1 ; c1ccc(-c2cccc3c2c2c(N(c4ccccc4)c4cccc(-c5cccc6ccccc56)c4)cccc2n3-c2ccccc2)cc1]

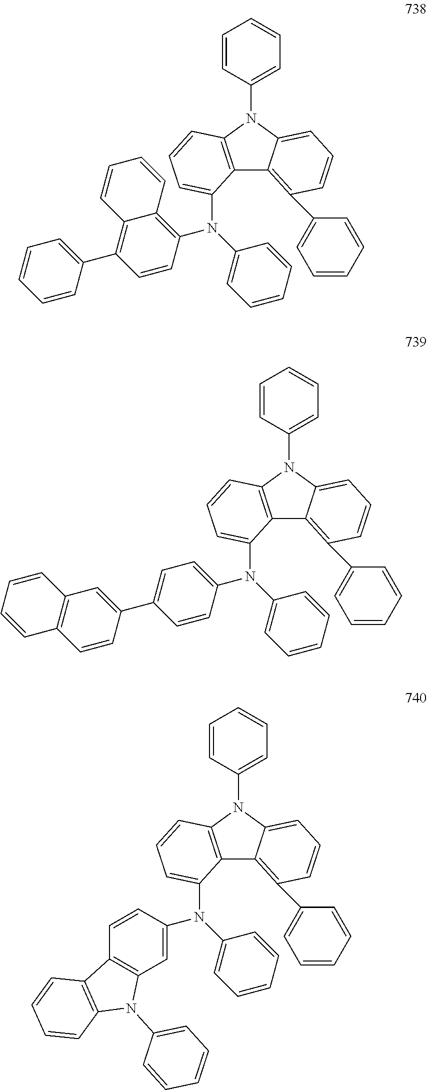 [SMILES: c1ccc(-c2ccc(N(c3ccccc3)c3cccc4c3c3c(-c5ccccc5)cccc3n4-c3ccccc3)c3ccccc23)cc1 ; c1ccc(-c2cccc3c2c2c(N(c4ccccc4)c4ccc(-c5ccc6ccccc6c5)cc4)cccc2n3-c2ccccc2)cc1 ; c1ccc(-c2cccc3c2c2c(N(c4ccccc4)c4ccc5c6ccccc6n(-c6ccccc6)c5c4)cccc2n3-c2ccccc2)cc1]

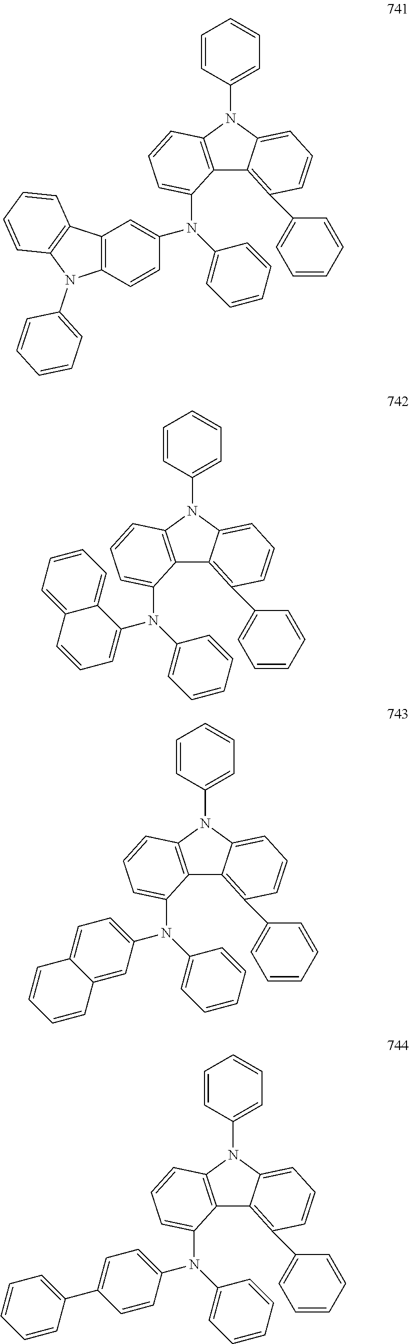

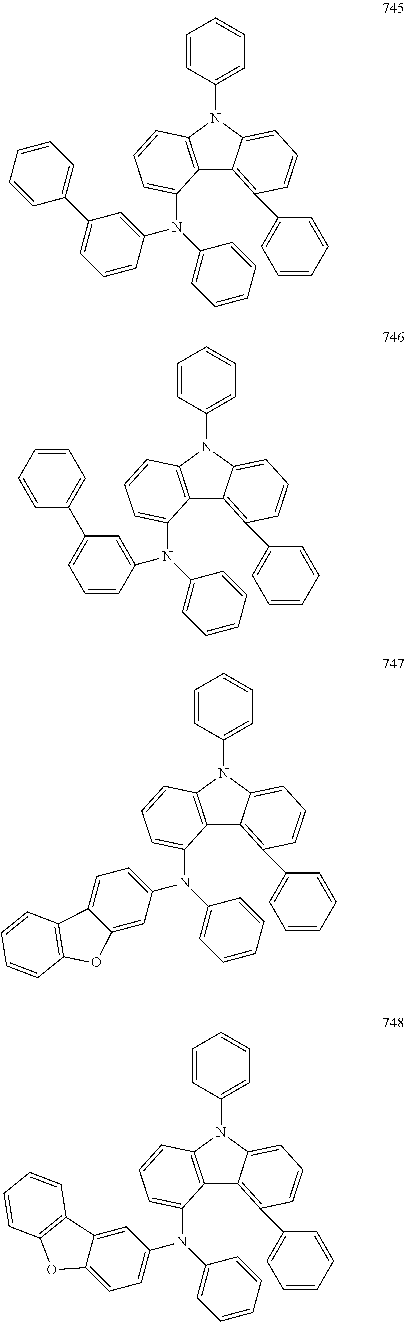

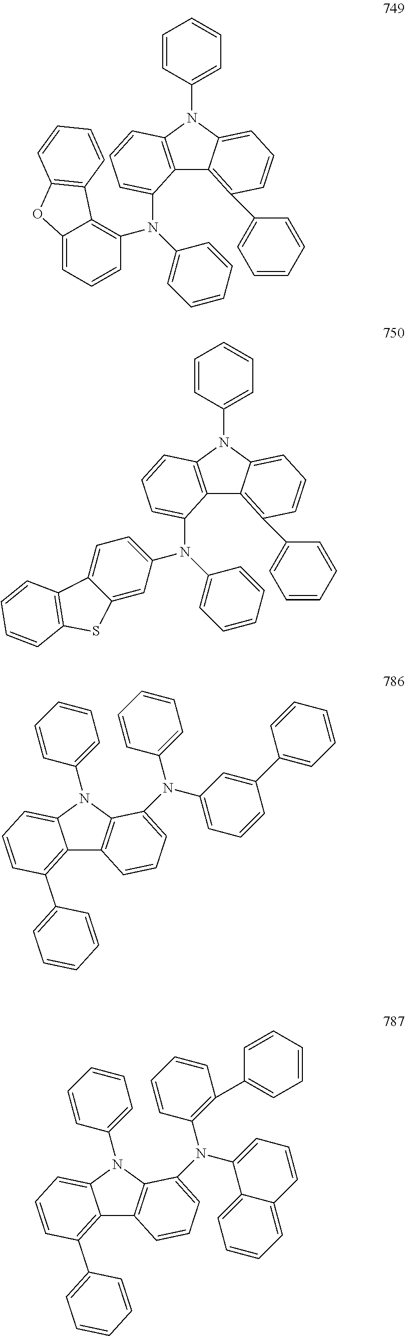

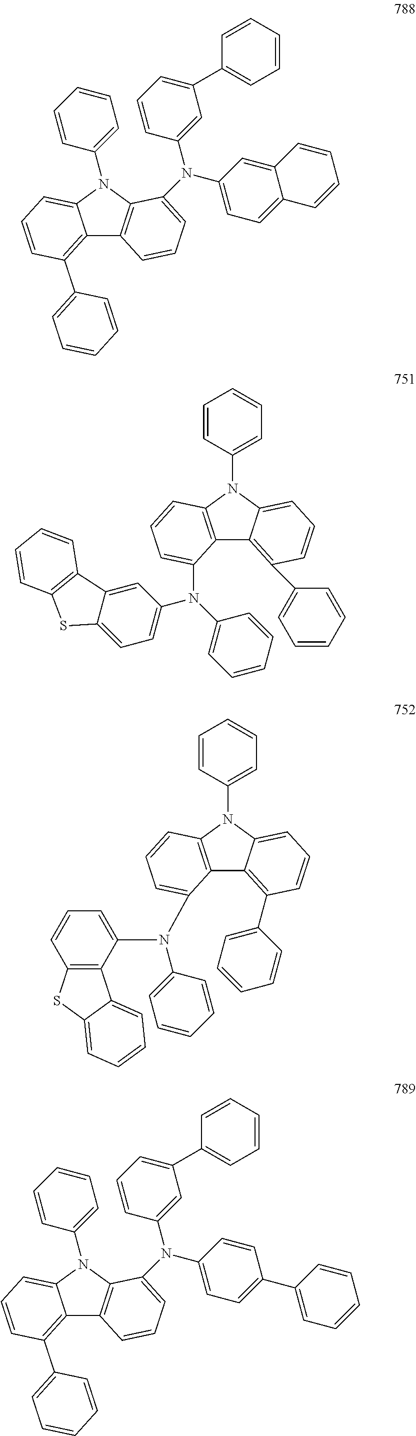

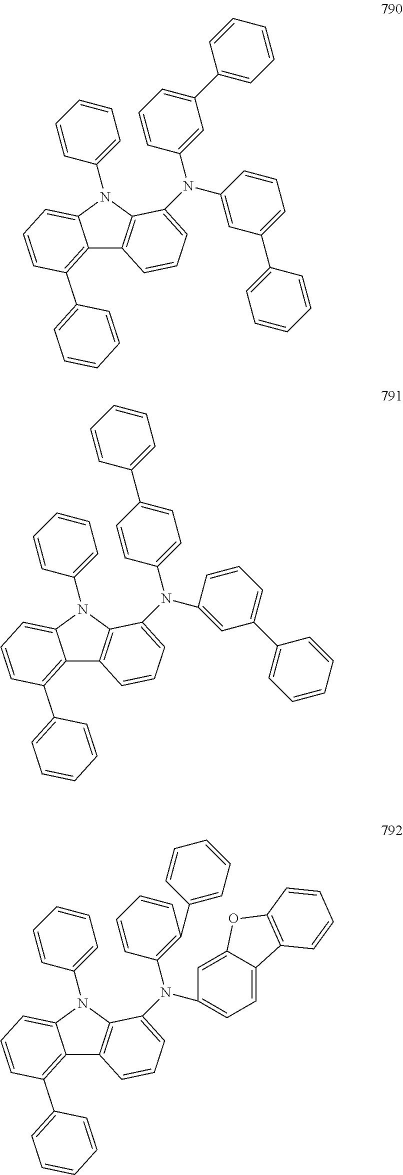

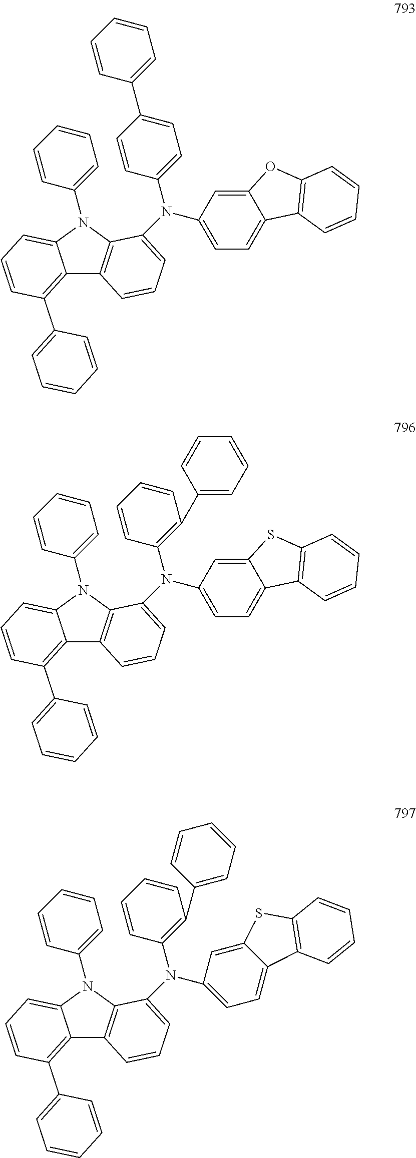 [SMILES: c1ccc(-c2ccc(N(c3ccc4c(c3)oc3ccccc34)c3cccc4c5c(-c6ccccc6)cccc5n(-c5ccccc5)c34)cc2)cc1 ; c1ccc(-c2ccccc2N(c2ccc3c(c2)sc2ccccc23)c2cccc3c4c(-c5ccccc5)cccc4n(-c4ccccc4)c23)cc1 ; c1ccc(-c2ccccc2N(c2ccc3c(c2)sc2ccccc23)c2cccc3c4c(-c5ccccc5)cccc4n(-c4ccccc4)c23)cc1]

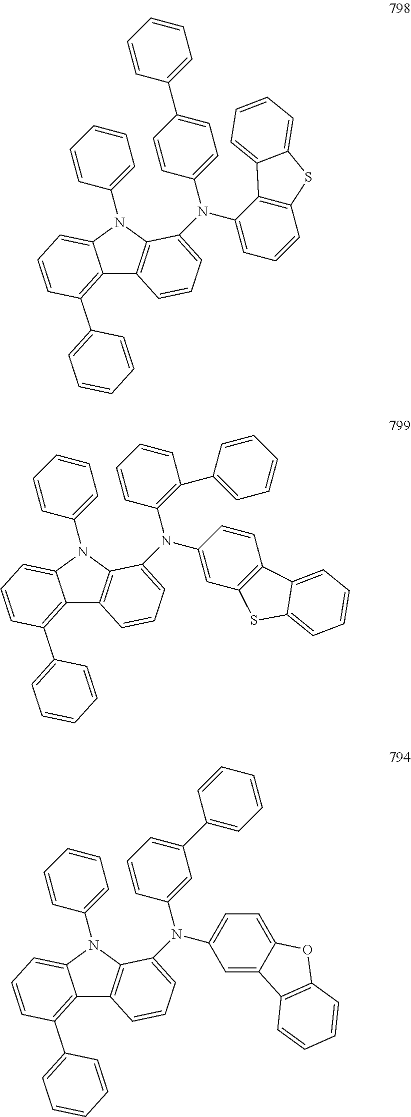 [SMILES: c1ccc(-c2ccc(N(c3cccc4sc5ccccc5c34)c3cccc4c5c(-c6ccccc6)cccc5n(-c5ccccc5)c34)cc2)cc1 ; c1ccc(-c2ccccc2N(c2ccc3c(c2)sc2ccccc23)c2cccc3c4c(-c5ccccc5)cccc4n(-c4ccccc4)c23)cc1 ; c1ccc(-c2cccc(N(c3ccc4oc5ccccc5c4c3)c3cccc4c5c(-c6ccccc6)cccc5n(-c5ccccc5)c34)c2)cc1]

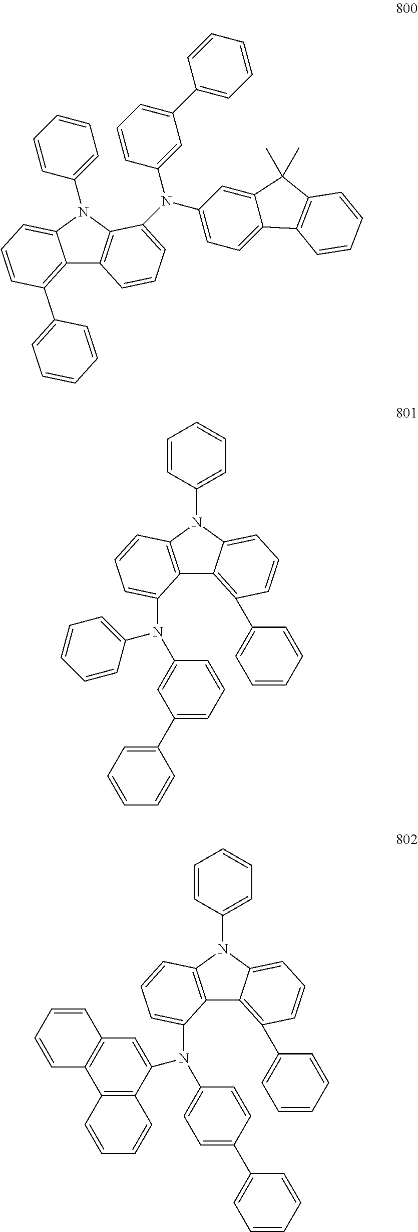 [SMILES: CC1(C)c2ccccc2-c2ccc(N(c3cccc(-c4ccccc4)c3)c3cccc4c5c(-c6ccccc6)cccc5n(-c5ccccc5)c34)cc21 ; c1ccc(-c2cccc(N(c3ccccc3)c3cccc4c3c3c(-c5ccccc5)cccc3n4-c3ccccc3)c2)cc1 ; c1ccc(-c2ccc(N(c3cc4ccccc4c4ccccc34)c3cccc4c3c3c(-c5ccccc5)cccc3n4-c3ccccc3)cc2)cc1]

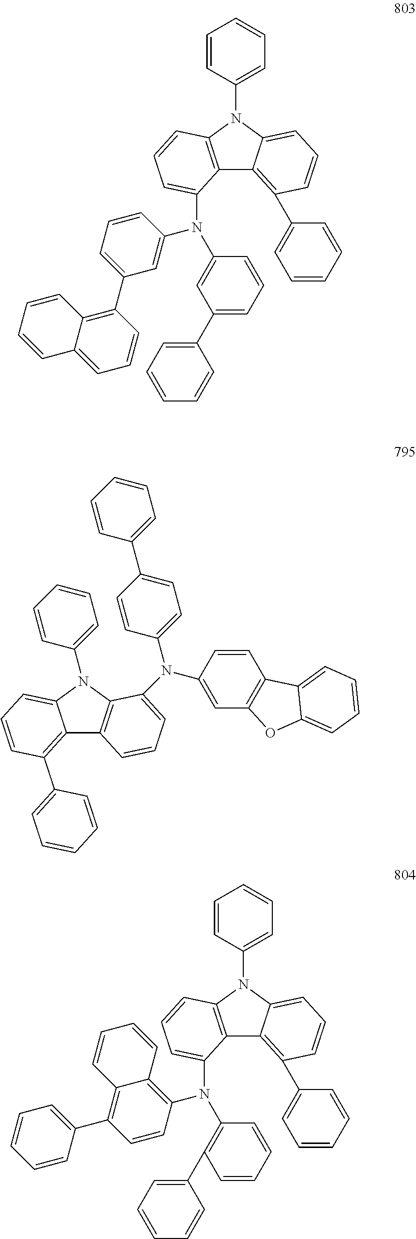 [SMILES: c1ccc(-c2cccc(N(c3cccc(-c4cccc5ccccc45)c3)c3cccc4c3c3c(-c5ccccc5)cccc3n4-c3ccccc3)c2)cc1 ; c1ccc(-c2ccc(N(c3ccc4c(c3)oc3ccccc34)c3cccc4c5c(-c6ccccc6)cccc5n(-c5ccccc5)c34)cc2)cc1 ; c1ccc(-c2ccccc2N(c2ccc(-c3ccccc3)c3ccccc23)c2cccc3c2c2c(-c4ccccc4)cccc2n3-c2ccccc2)cc1]

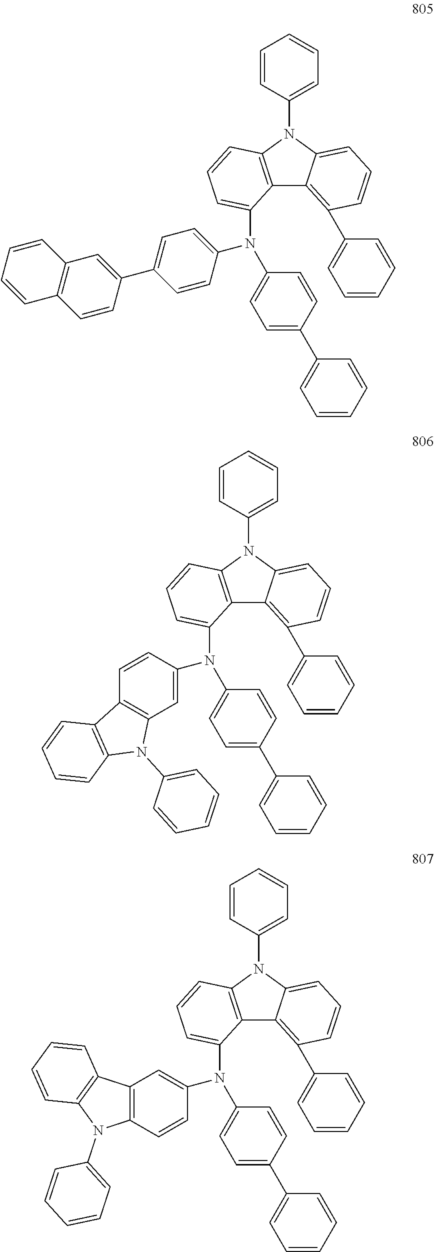

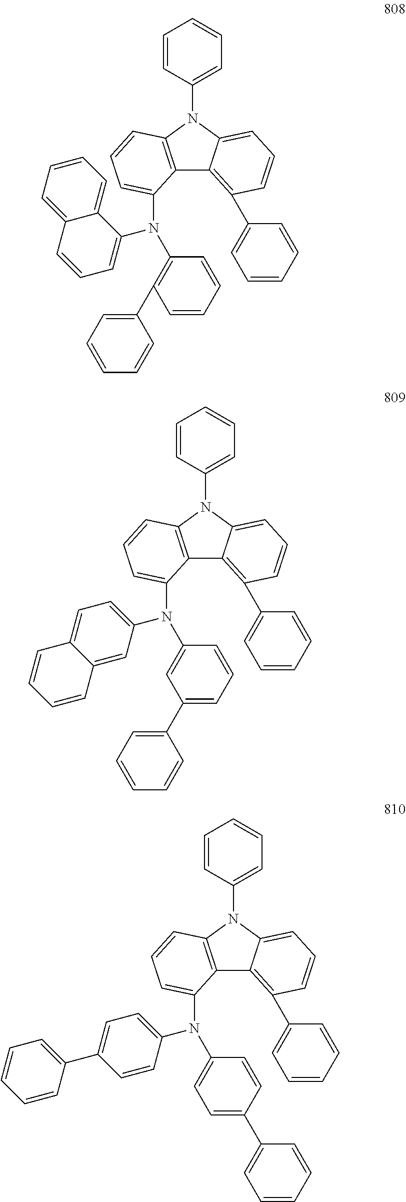 [SMILES: c1ccc(-c2ccccc2N(c2cccc3ccccc23)c2cccc3c2c2c(-c4ccccc4)cccc2n3-c2ccccc2)cc1 ; c1ccc(-c2cccc(N(c3ccc4ccccc4c3)c3cccc4c3c3c(-c5ccccc5)cccc3n4-c3ccccc3)c2)cc1 ; c1ccc(-c2ccc(N(c3ccc(-c4ccccc4)cc3)c3cccc4c3c3c(-c5ccccc5)cccc3n4-c3ccccc3)cc2)cc1]

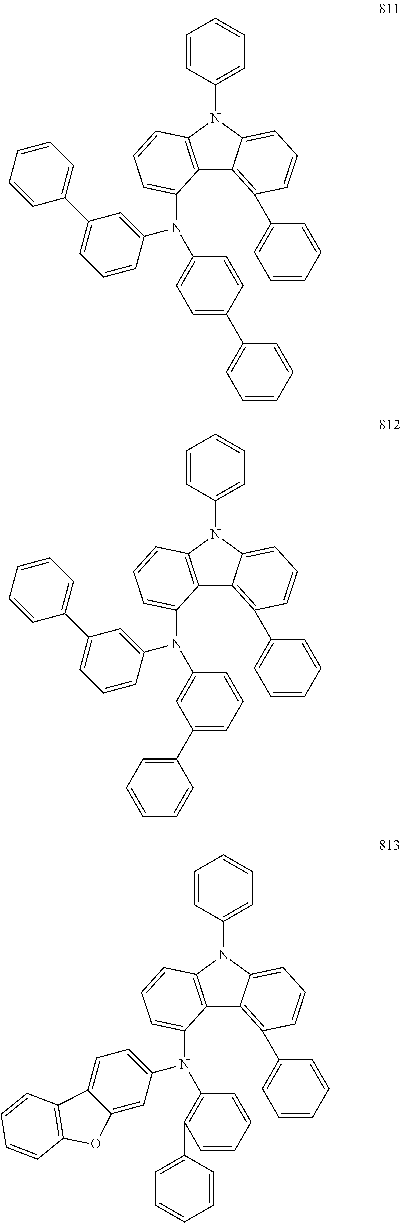

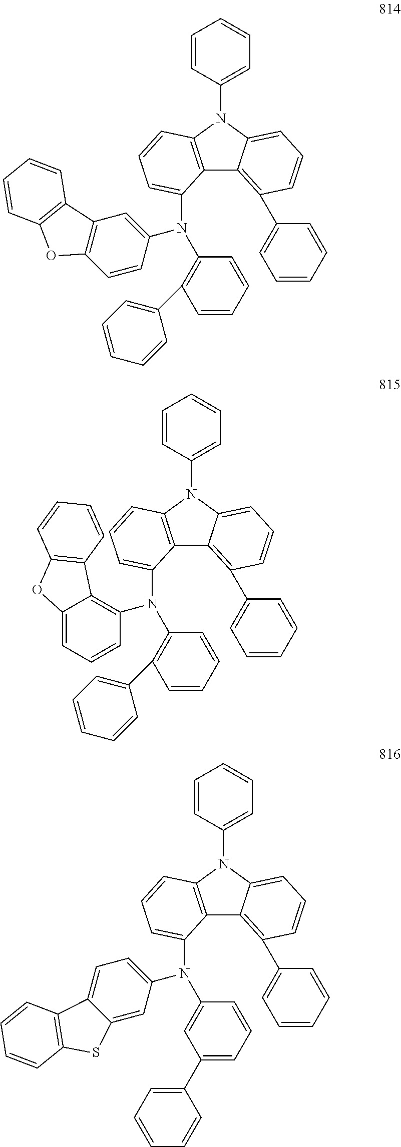 [SMILES: c1ccc(-c2ccccc2N(c2ccc3oc4ccccc4c3c2)c2cccc3c2c2c(-c4ccccc4)cccc2n3-c2ccccc2)cc1 ; c1ccc(-c2ccccc2N(c2cccc3oc4ccccc4c23)c2cccc3c2c2c(-c4ccccc4)cccc2n3-c2ccccc2)cc1 ; c1ccc(-c2cccc(N(c3ccc4c(c3)sc3ccccc34)c3cccc4c3c3c(-c5ccccc5)cccc3n4-c3ccccc3)c2)cc1]

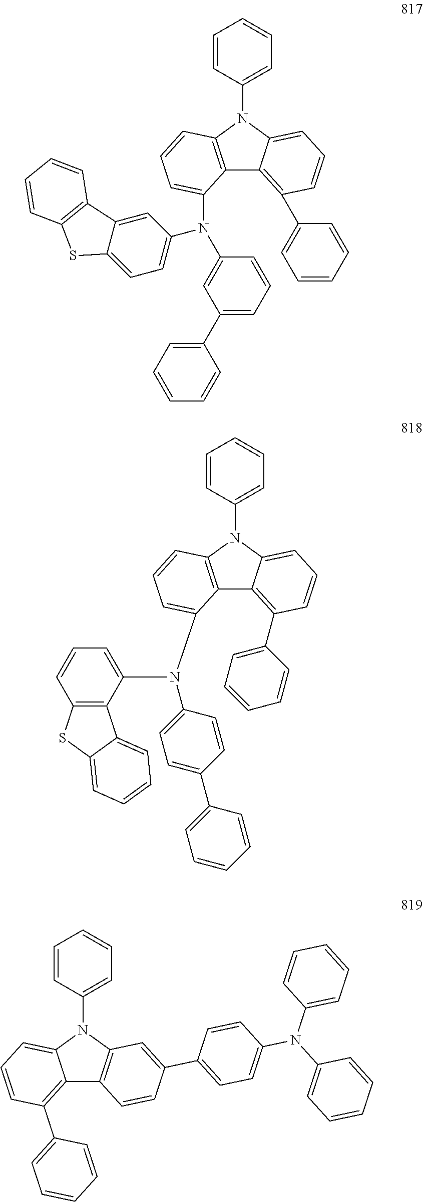 [SMILES: c1ccc(-c2cccc(N(c3ccc4sc5ccccc5c4c3)c3cccc4c3c3c(-c5ccccc5)cccc3n4-c3ccccc3)c2)cc1 ; c1ccc(-c2ccc(N(c3cccc4sc5ccccc5c34)c3cccc4c3c3c(-c5ccccc5)cccc3n4-c3ccccc3)cc2)cc1 ; c1ccc(-c2cccc3c2c2ccc(-c4ccc(N(c5ccccc5)c5ccccc5)cc4)cc2n3-c2ccccc2)cc1]

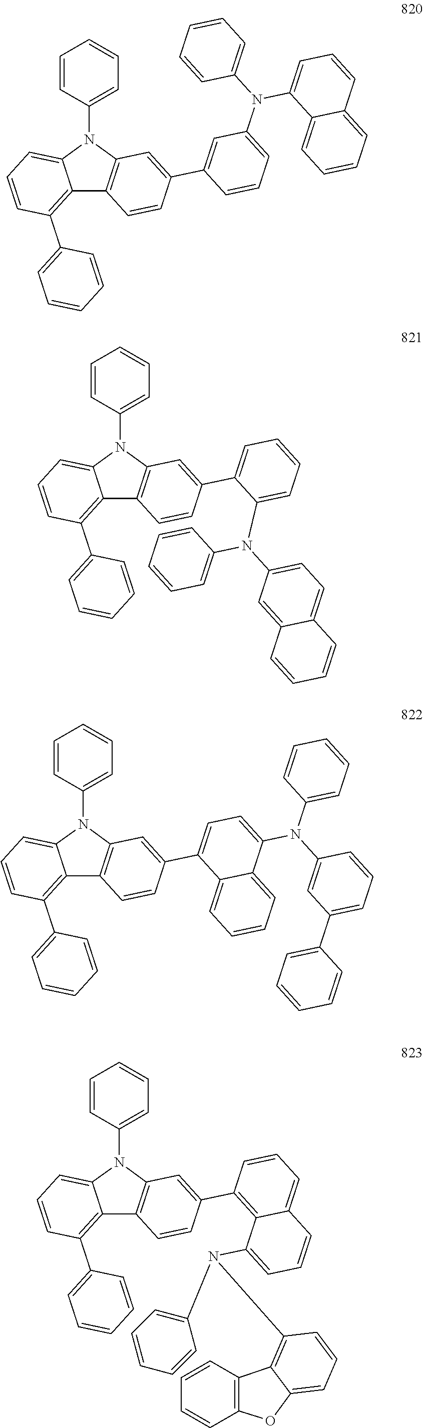 [SMILES: c1ccc(-c2cccc3c2c2ccc(-c4cccc(N(c5ccccc5)c5cccc6ccccc56)c4)cc2n3-c2ccccc2)cc1 ; c1ccc(-c2cccc3c2c2ccc(-c4ccccc4N(c4ccccc4)c4ccc5ccccc5c4)cc2n3-c2ccccc2)cc1 ; c1ccc(-c2cccc(N(c3ccccc3)c3ccc(-c4ccc5c6c(-c7ccccc7)cccc6n(-c6ccccc6)c5c4)c4ccccc34)c2)cc1 ; c1ccc(-c2cccc3c2c2ccc(-c4cccc5cccc(N(c6ccccc6)c6cccc7oc8ccccc8c67)c45)cc2n3-c2ccccc2)cc1]

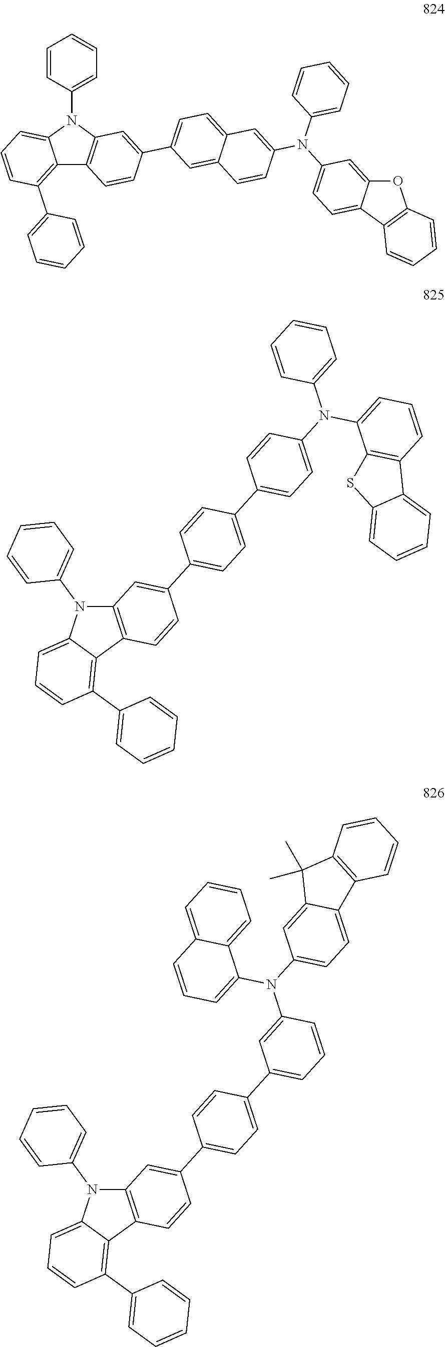 [SMILES: c1ccc(-c2cccc3c2c2ccc(-c4ccc5cc(N(c6ccccc6)c6ccc7c(c6)oc6ccccc67)ccc5c4)cc2n3-c2ccccc2)cc1 ; c1ccc(-c2cccc3c2c2ccc(-c4ccc(-c5ccc(N(c6ccccc6)c6cccc7c6sc6ccccc67)cc5)cc4)cc2n3-c2ccccc2)cc1 ; CC1(C)c2ccccc2-c2ccc(N(c3cccc(-c4ccc(-c5ccc6c7c(-c8ccccc8)cccc7n(-c7ccccc7)c6c5)cc4)c3)c3cccc4ccccc34)cc21]

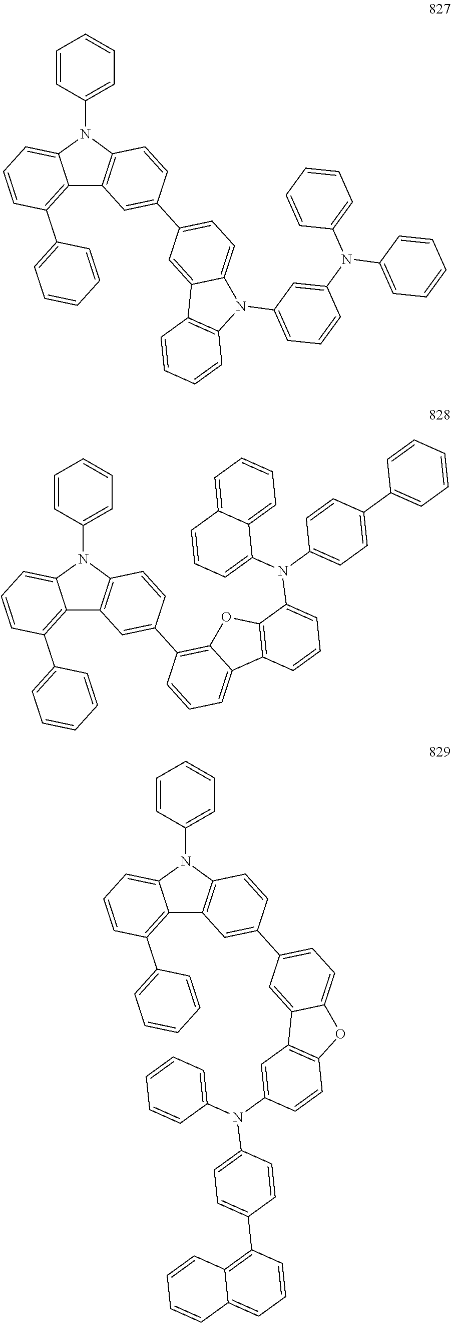 [SMILES: c1ccc(-c2cccc3c2c2cc(-c4ccc5c(c4)c4ccccc4n5-c4cccc(N(c5ccccc5)c5ccccc5)c4)ccc2n3-c2ccccc2)cc1 ; c1ccc(-c2ccc(N(c3cccc4ccccc34)c3cccc4c3oc3c(-c5ccc6c(c5)c5c(-c7ccccc7)cccc5n6-c5ccccc5)cccc34)cc2)cc1 ; c1ccc(-c2cccc3c2c2cc(-c4ccc5oc6ccc(N(c7ccccc7)c7ccc(-c8cccc9ccccc89)cc7)cc6c5c4)ccc2n3-c2ccccc2)cc1]

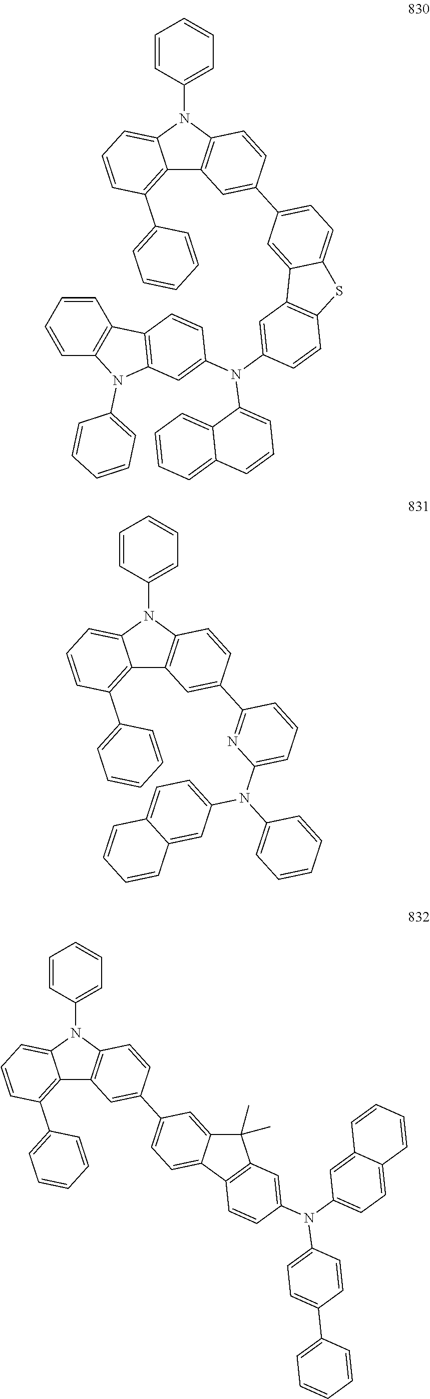 [SMILES: c1ccc(-c2cccc3c2c2cc(-c4ccc5sc6ccc(N(c7ccc8c9ccccc9n(-c9ccccc9)c8c7)c7cccc8ccccc78)cc6c5c4)ccc2n3-c2ccccc2)cc1 ; c1ccc(-c2cccc3c2c2cc(-c4cccc(N(c5ccccc5)c5ccc6ccccc6c5)n4)ccc2n3-c2ccccc2)cc1 ; CC1(C)c2cc(-c3ccc4c(c3)c3c(-c5ccccc5)cccc3n4-c3ccccc3)ccc2-c2ccc(N(c3ccc(-c4ccccc4)cc3)c3ccc4ccccc4c3)cc21]

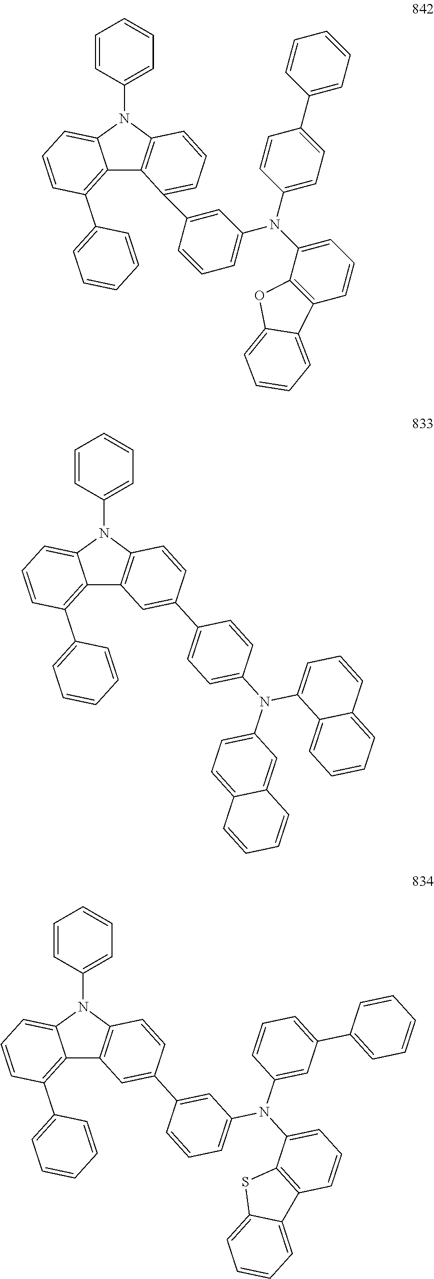 [SMILES: c1ccc(-c2ccc(N(c3cccc(-c4cccc5c4c4c(-c6ccccc6)cccc4n5-c4ccccc4)c3)c3cccc4c3oc3ccccc34)cc2)cc1 ; c1ccc(-c2cccc3c2c2cc(-c4ccc(N(c5ccc6ccccc6c5)c5cccc6ccccc56)cc4)ccc2n3-c2ccccc2)cc1 ; c1ccc(-c2cccc(N(c3cccc(-c4ccc5c(c4)c4c(-c6ccccc6)cccc4n5-c4ccccc4)c3)c3cccc4c3sc3ccccc34)c2)cc1]

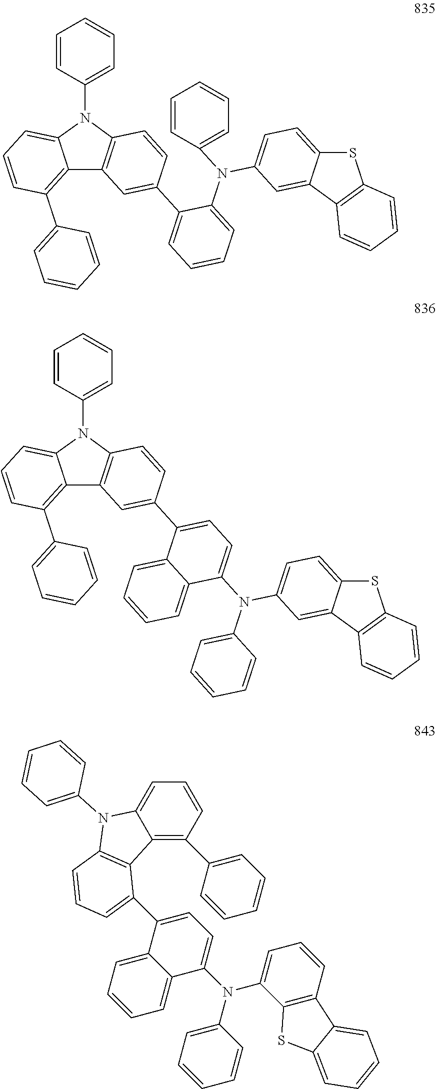

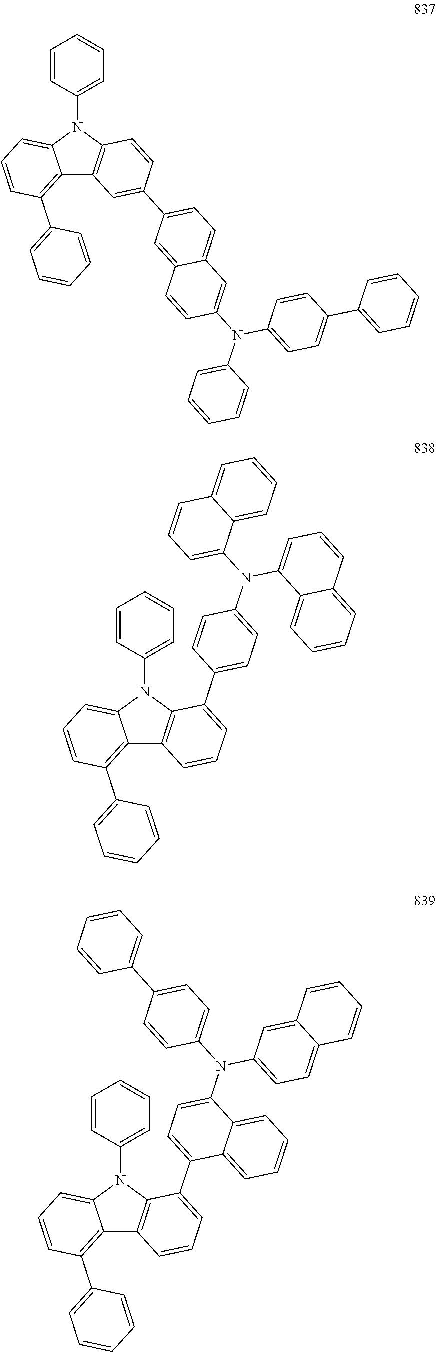

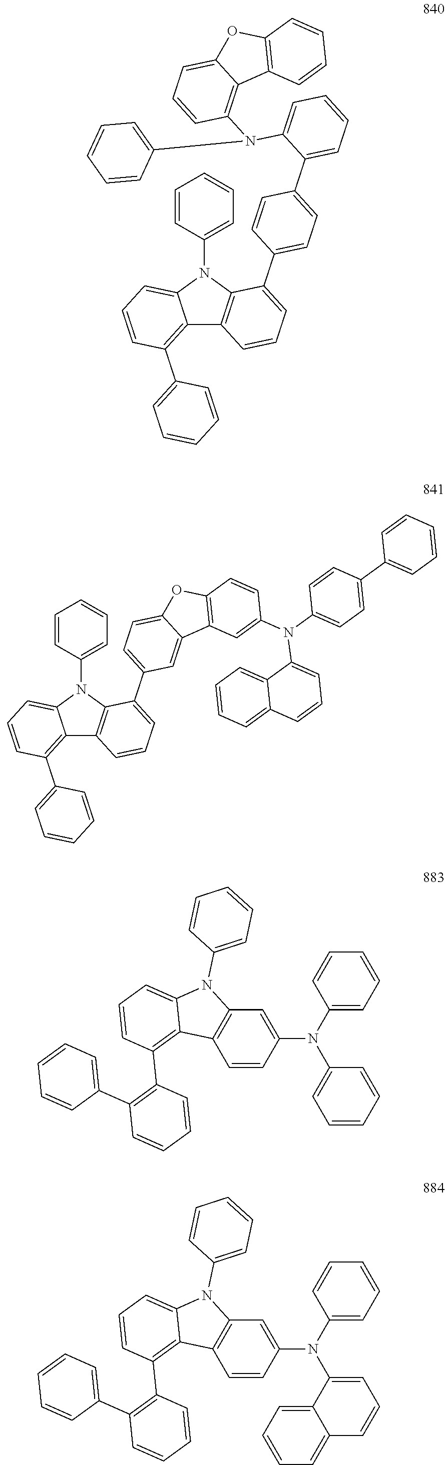

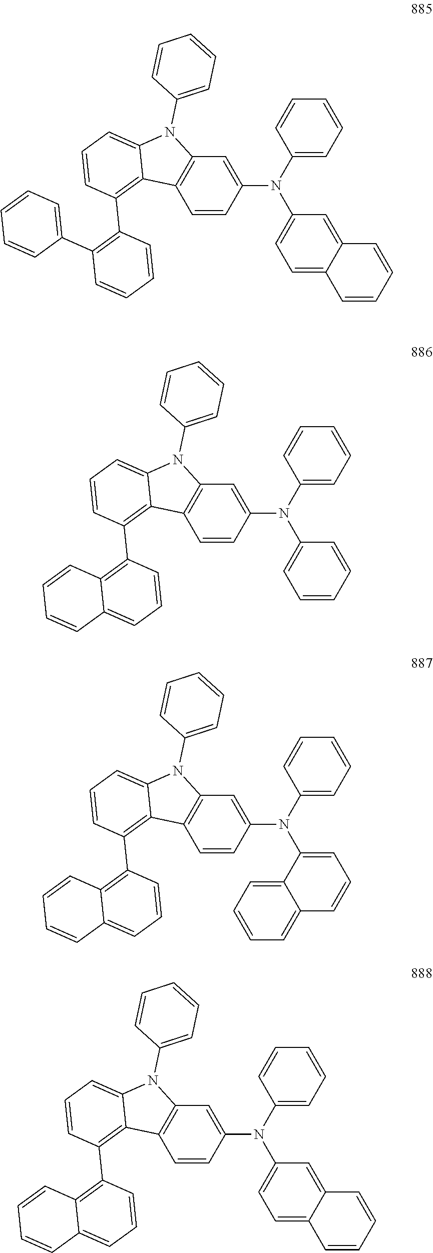 [SMILES: c1ccc(-c2ccccc2-c2cccc3c2c2ccc(N(c4ccccc4)c4ccc5ccccc5c4)cc2n3-c2ccccc2)cc1 ; c1ccc(N(c2ccccc2)c2ccc3c4c(-c5cccc6ccccc56)cccc4n(-c4ccccc4)c3c2)cc1 ; c1ccc(N(c2ccc3c4c(-c5cccc6ccccc56)cccc4n(-c4ccccc4)c3c2)c2cccc3ccccc23)cc1 ; c1ccc(N(c2ccc3ccccc3c2)c2ccc3c4c(-c5cccc6ccccc56)cccc4n(-c4ccccc4)c3c2)cc1]

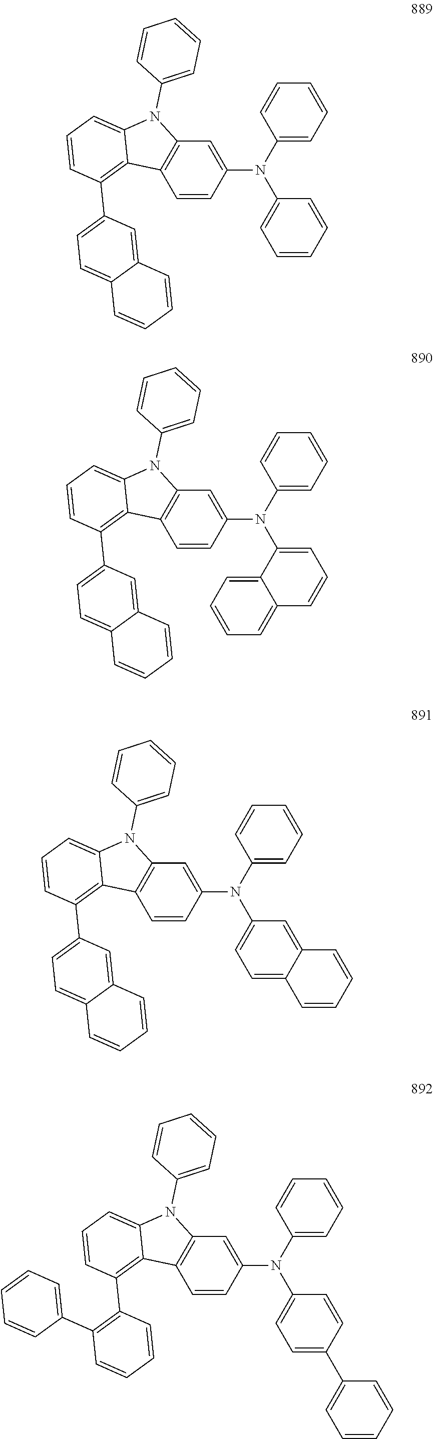 [SMILES: c1ccc(N(c2ccccc2)c2ccc3c4c(-c5ccc6ccccc6c5)cccc4n(-c4ccccc4)c3c2)cc1 ; c1ccc(N(c2ccc3c4c(-c5ccc6ccccc6c5)cccc4n(-c4ccccc4)c3c2)c2cccc3ccccc23)cc1 ; c1ccc(N(c2ccc3ccccc3c2)c2ccc3c4c(-c5ccc6ccccc6c5)cccc4n(-c4ccccc4)c3c2)cc1 ; c1ccc(-c2ccc(N(c3ccccc3)c3ccc4c5c(-c6ccccc6-c6ccccc6)cccc5n(-c5ccccc5)c4c3)cc2)cc1]

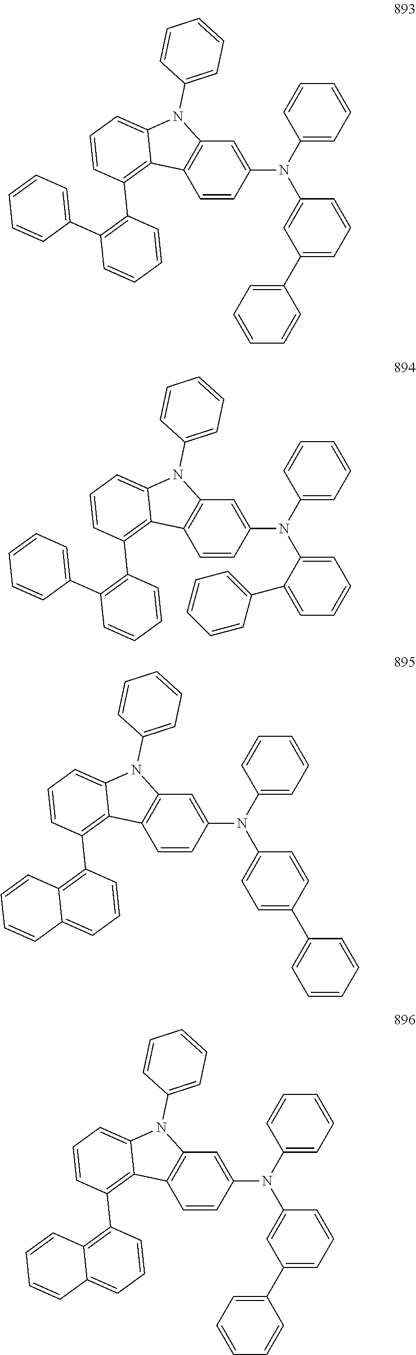

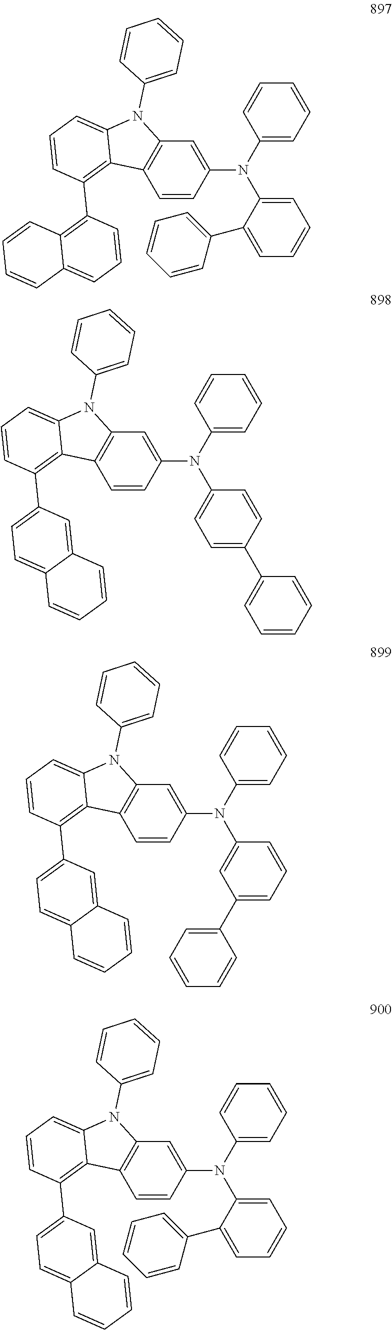

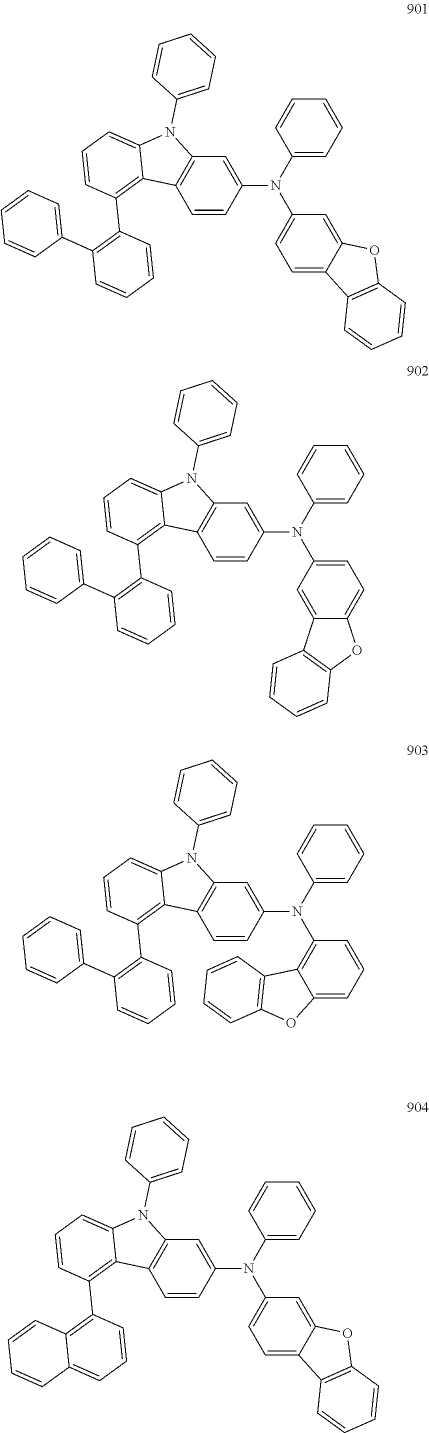 [SMILES: c1ccc(-c2ccccc2-c2cccc3c2c2ccc(N(c4ccccc4)c4ccc5c(c4)oc4ccccc45)cc2n3-c2ccccc2)cc1 ; c1ccc(-c2ccccc2-c2cccc3c2c2ccc(N(c4ccccc4)c4ccc5oc6ccccc6c5c4)cc2n3-c2ccccc2)cc1 ; c1ccc(-c2ccccc2-c2cccc3c2c2ccc(N(c4ccccc4)c4cccc5oc6ccccc6c45)cc2n3-c2ccccc2)cc1 ; c1ccc(N(c2ccc3c(c2)oc2ccccc23)c2ccc3c4c(-c5cccc6ccccc56)cccc4n(-c4ccccc4)c3c2)cc1]

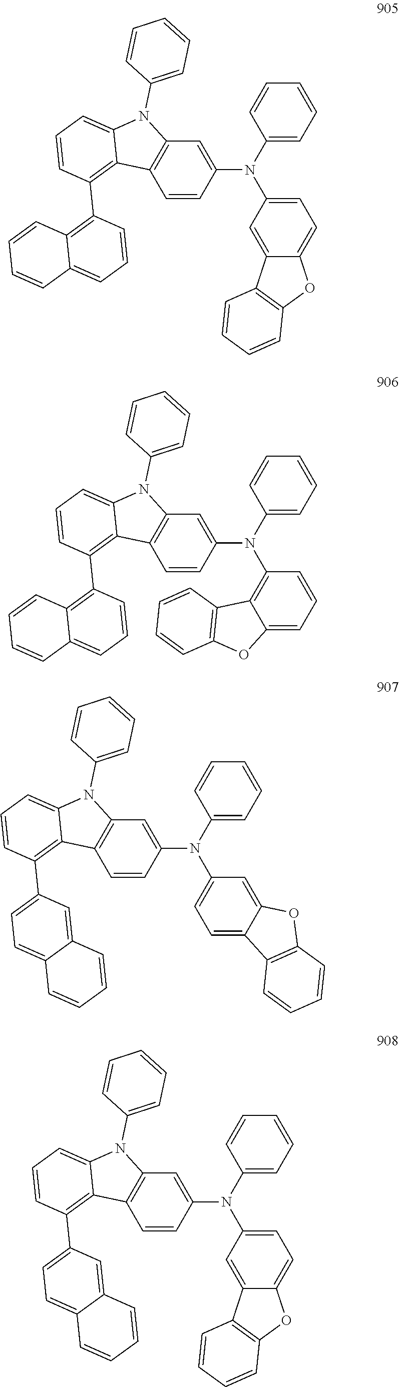

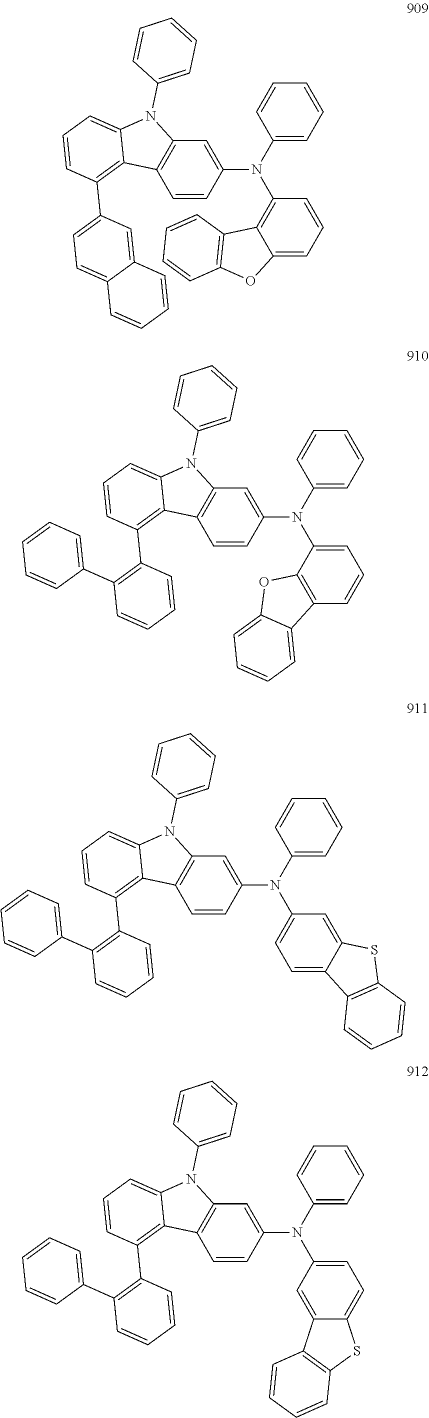 [SMILES: c1ccc(N(c2ccc3c4c(-c5ccc6ccccc6c5)cccc4n(-c4ccccc4)c3c2)c2cccc3oc4ccccc4c23)cc1 ; c1ccc(-c2ccccc2-c2cccc3c2c2ccc(N(c4ccccc4)c4cccc5c4oc4ccccc45)cc2n3-c2ccccc2)cc1 ; c1ccc(-c2ccccc2-c2cccc3c2c2ccc(N(c4ccccc4)c4ccc5c(c4)sc4ccccc45)cc2n3-c2ccccc2)cc1 ; c1ccc(-c2ccccc2-c2cccc3c2c2ccc(N(c4ccccc4)c4ccc5sc6ccccc6c5c4)cc2n3-c2ccccc2)cc1]

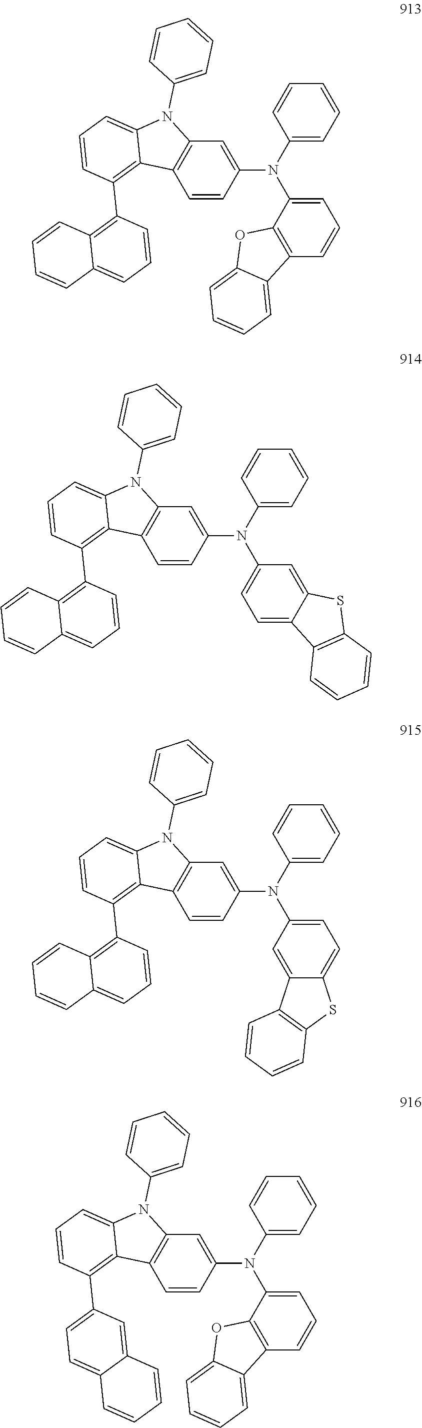 [SMILES: c1ccc(N(c2ccc3c4c(-c5cccc6ccccc56)cccc4n(-c4ccccc4)c3c2)c2cccc3c2oc2ccccc23)cc1 ; c1ccc(N(c2ccc3c(c2)sc2ccccc23)c2ccc3c4c(-c5cccc6ccccc56)cccc4n(-c4ccccc4)c3c2)cc1 ; c1ccc(N(c2ccc3sc4ccccc4c3c2)c2ccc3c4c(-c5cccc6ccccc56)cccc4n(-c4ccccc4)c3c2)cc1 ; c1ccc(N(c2ccc3c4c(-c5ccc6ccccc6c5)cccc4n(-c4ccccc4)c3c2)c2cccc3c2oc2ccccc23)cc1]

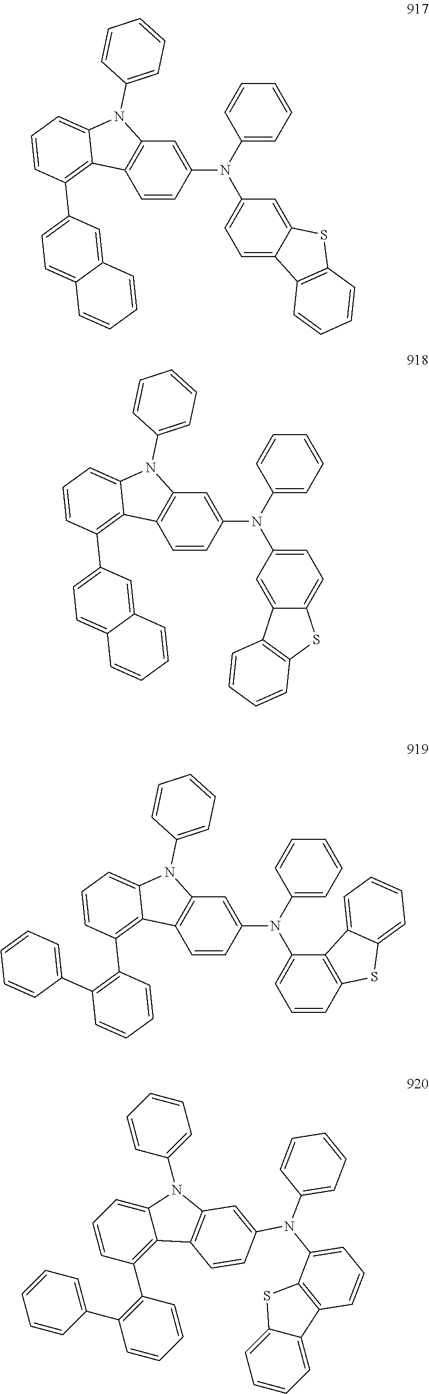 [SMILES: c1ccc(N(c2ccc3c(c2)sc2ccccc23)c2ccc3c4c(-c5ccc6ccccc6c5)cccc4n(-c4ccccc4)c3c2)cc1 ; c1ccc(N(c2ccc3sc4ccccc4c3c2)c2ccc3c4c(-c5ccc6ccccc6c5)cccc4n(-c4ccccc4)c3c2)cc1 ; c1ccc(-c2ccccc2-c2cccc3c2c2ccc(N(c4ccccc4)c4cccc5sc6ccccc6c45)cc2n3-c2ccccc2)cc1 ; c1ccc(-c2ccccc2-c2cccc3c2c2ccc(N(c4ccccc4)c4cccc5c4sc4ccccc45)cc2n3-c2ccccc2)cc1]

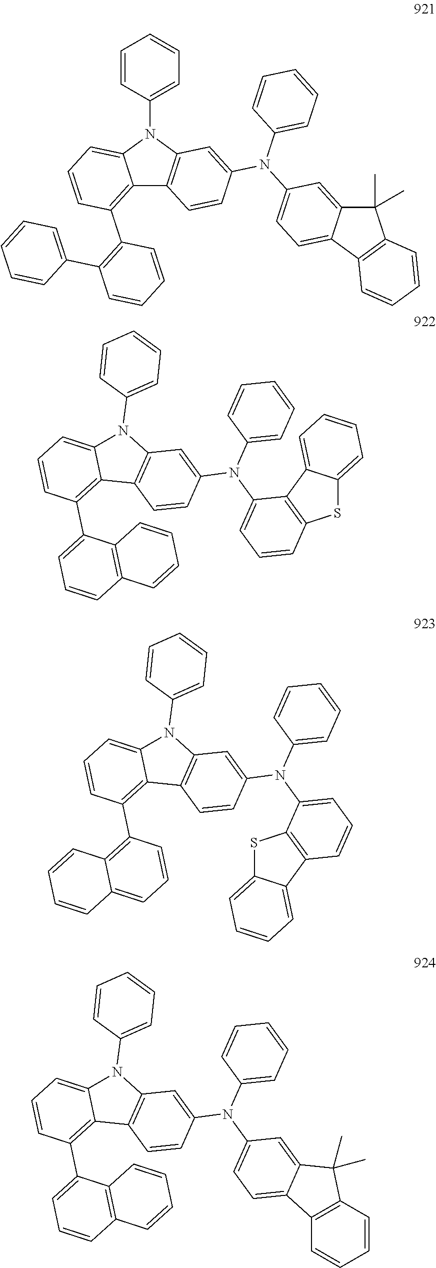

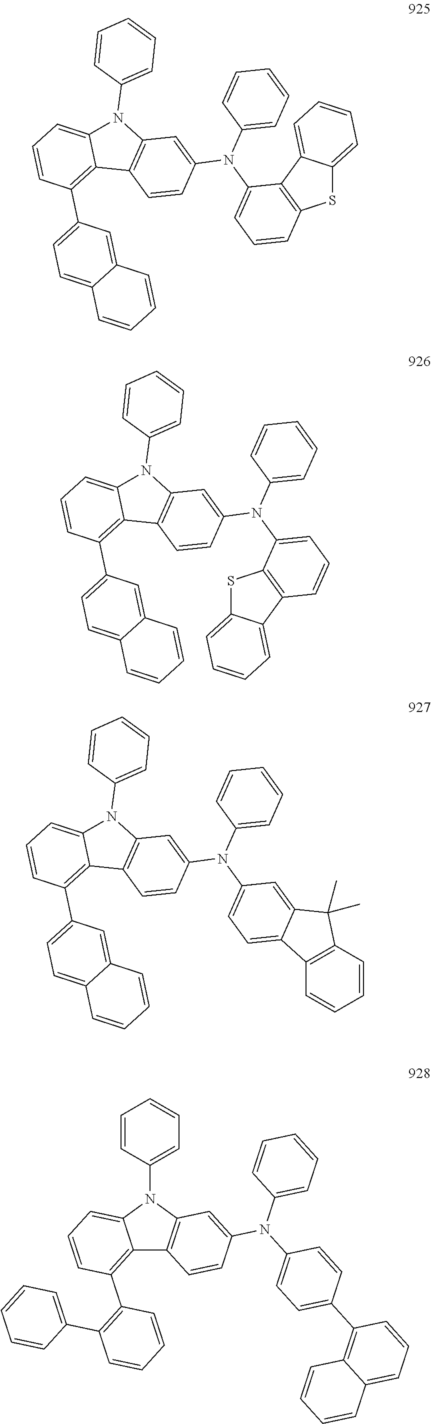

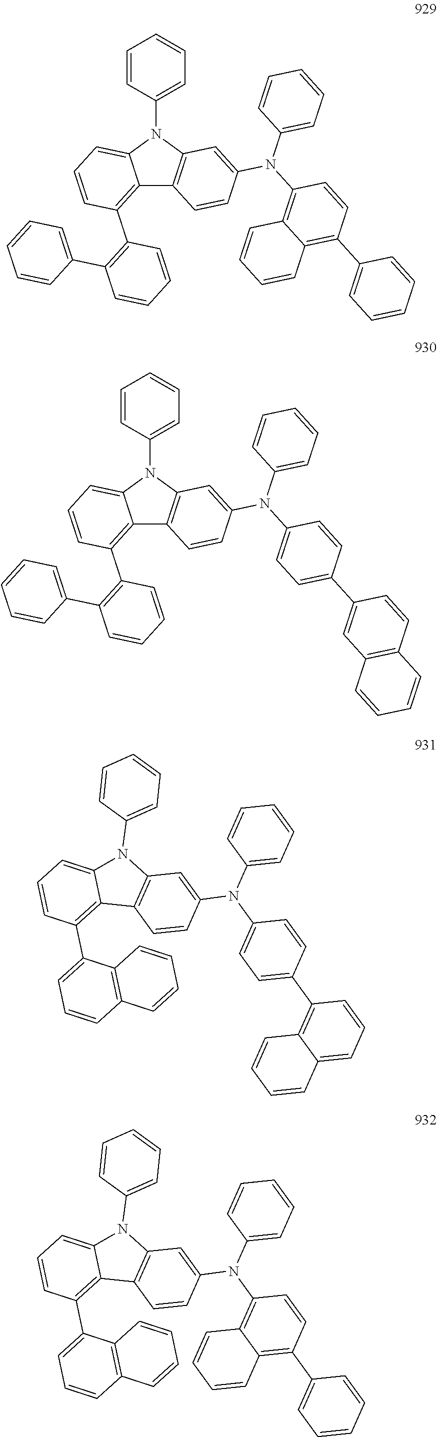 [SMILES: c1ccc(-c2ccccc2-c2cccc3c2c2ccc(N(c4ccccc4)c4ccc(-c5ccccc5)c5ccccc45)cc2n3-c2ccccc2)cc1 ; c1ccc(-c2ccccc2-c2cccc3c2c2ccc(N(c4ccccc4)c4ccc(-c5ccc6ccccc6c5)cc4)cc2n3-c2ccccc2)cc1 ; c1ccc(N(c2ccc(-c3cccc4ccccc34)cc2)c2ccc3c4c(-c5cccc6ccccc56)cccc4n(-c4ccccc4)c3c2)cc1 ; c1ccc(-c2ccc(N(c3ccccc3)c3ccc4c5c(-c6cccc7ccccc67)cccc5n(-c5ccccc5)c4c3)c3ccccc23)cc1]

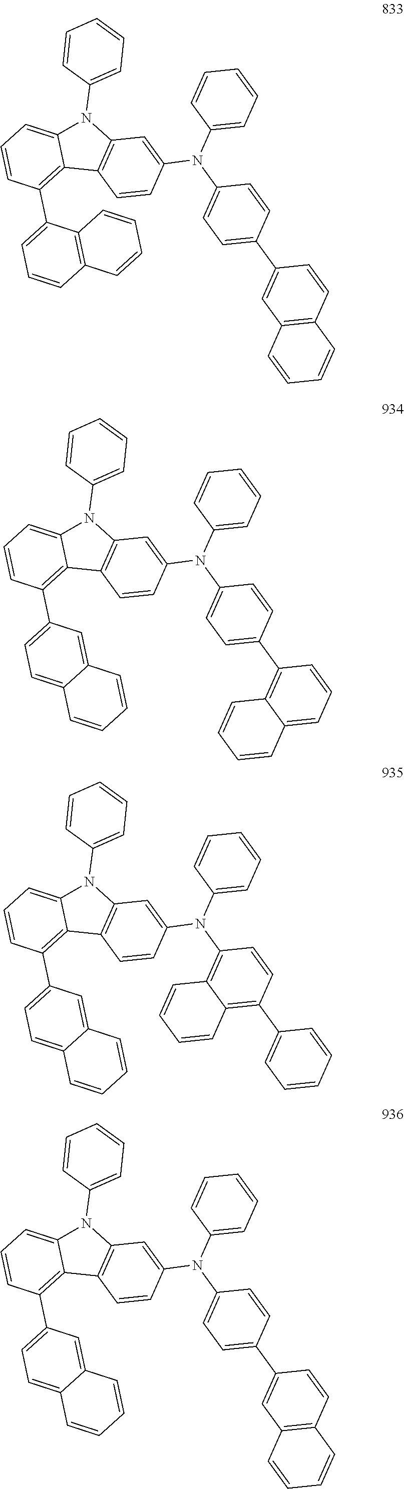

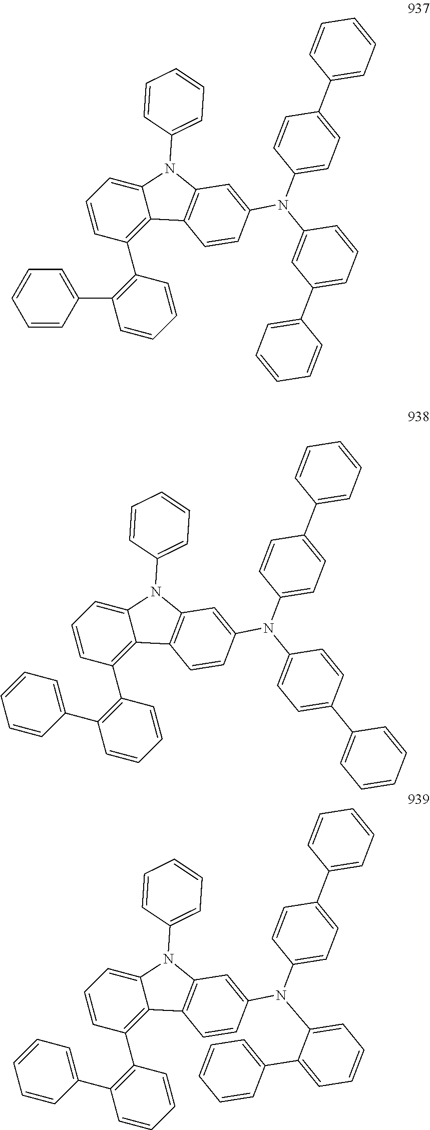

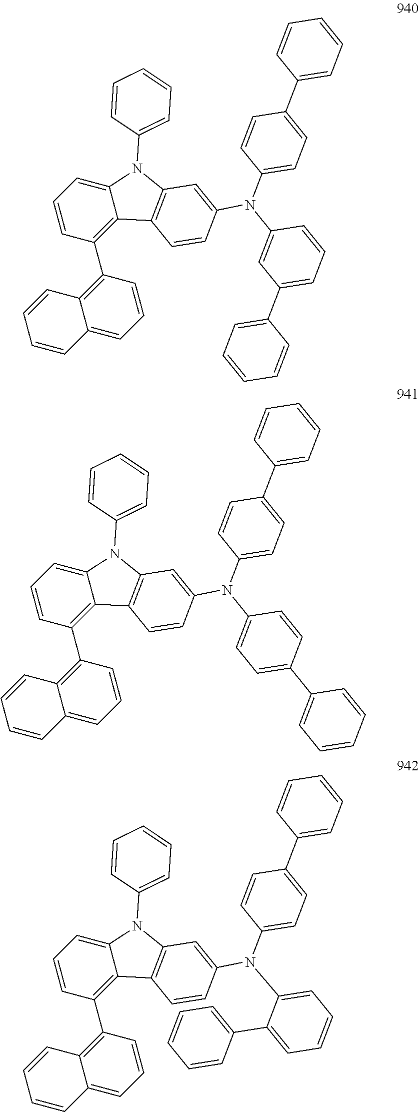

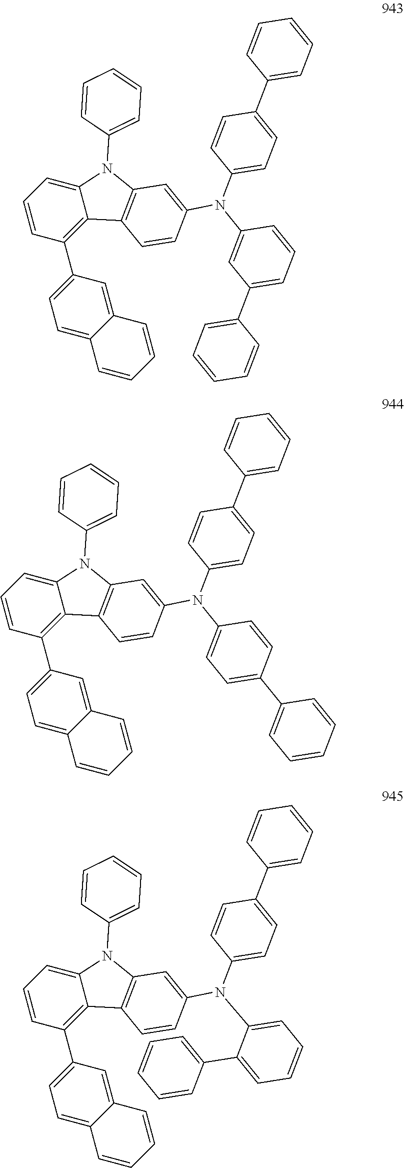

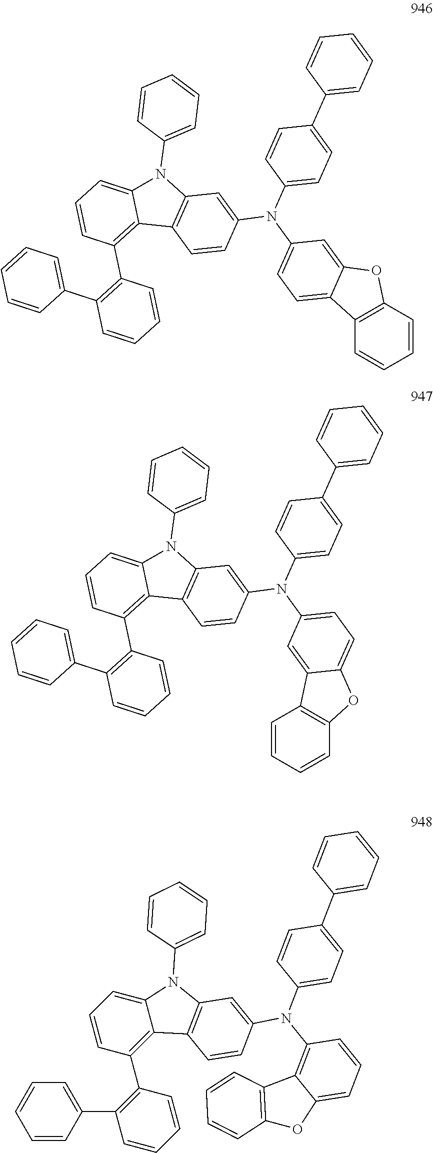

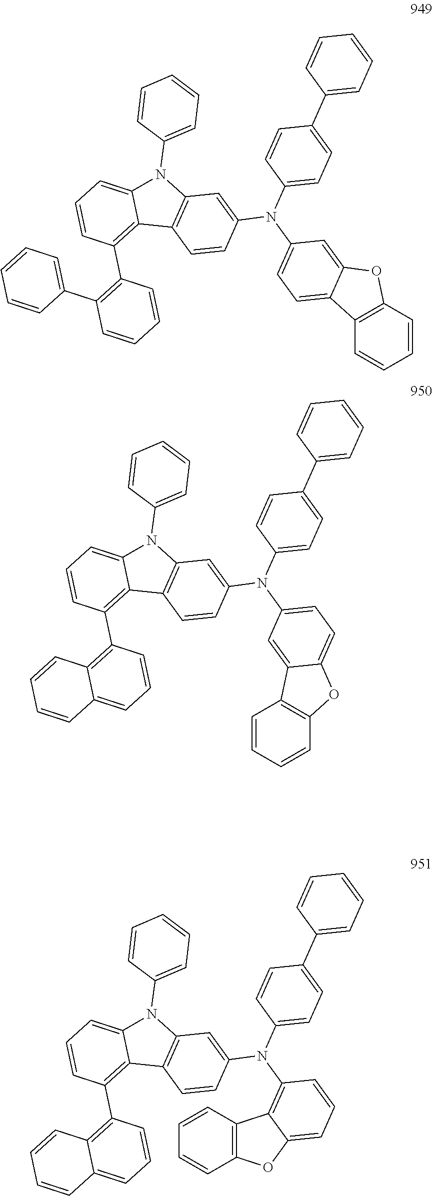 [SMILES: c1ccc(-c2ccc(N(c3ccc4c(c3)oc3ccccc34)c3ccc4c5c(-c6ccccc6-c6ccccc6)cccc5n(-c5ccccc5)c4c3)cc2)cc1 ; c1ccc(-c2ccc(N(c3ccc4oc5ccccc5c4c3)c3ccc4c5c(-c6cccc7ccccc67)cccc5n(-c5ccccc5)c4c3)cc2)cc1 ; c1ccc(-c2ccc(N(c3ccc4c5c(-c6cccc7ccccc67)cccc5n(-c5ccccc5)c4c3)c3cccc4oc5ccccc5c34)cc2)cc1]

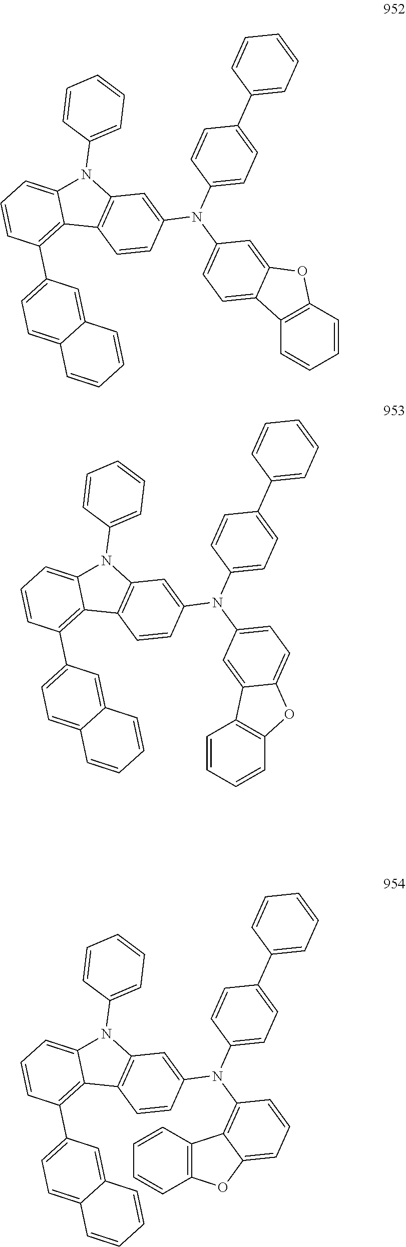 [SMILES: c1ccc(-c2ccc(N(c3ccc4c(c3)oc3ccccc34)c3ccc4c5c(-c6ccc7ccccc7c6)cccc5n(-c5ccccc5)c4c3)cc2)cc1 ; c1ccc(-c2ccc(N(c3ccc4oc5ccccc5c4c3)c3ccc4c5c(-c6ccc7ccccc7c6)cccc5n(-c5ccccc5)c4c3)cc2)cc1 ; c1ccc(-c2ccc(N(c3ccc4c5c(-c6ccc7ccccc7c6)cccc5n(-c5ccccc5)c4c3)c3cccc4oc5ccccc5c34)cc2)cc1]

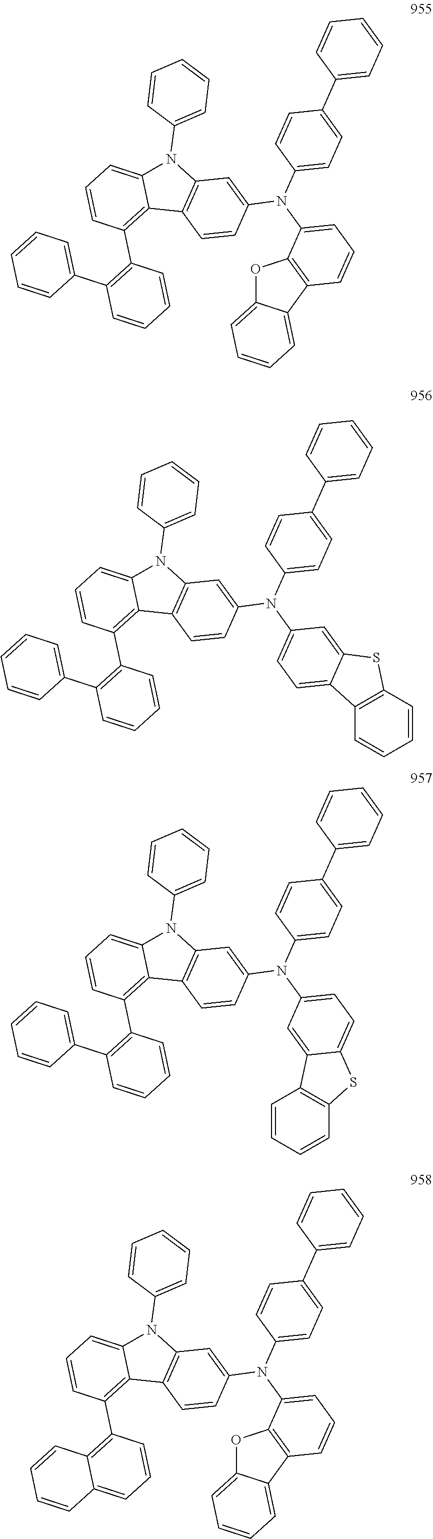

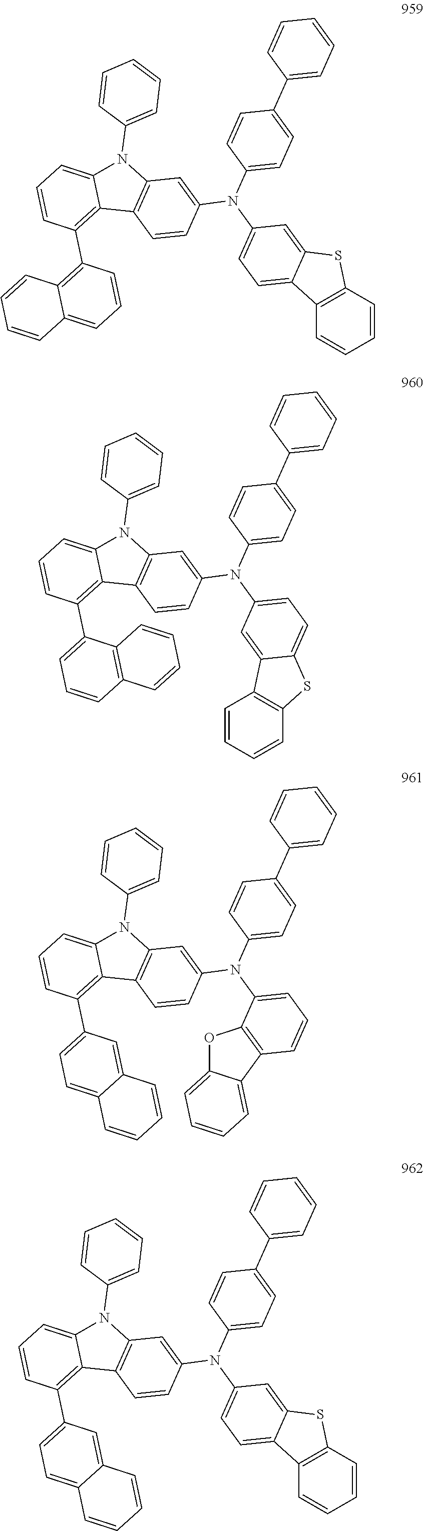

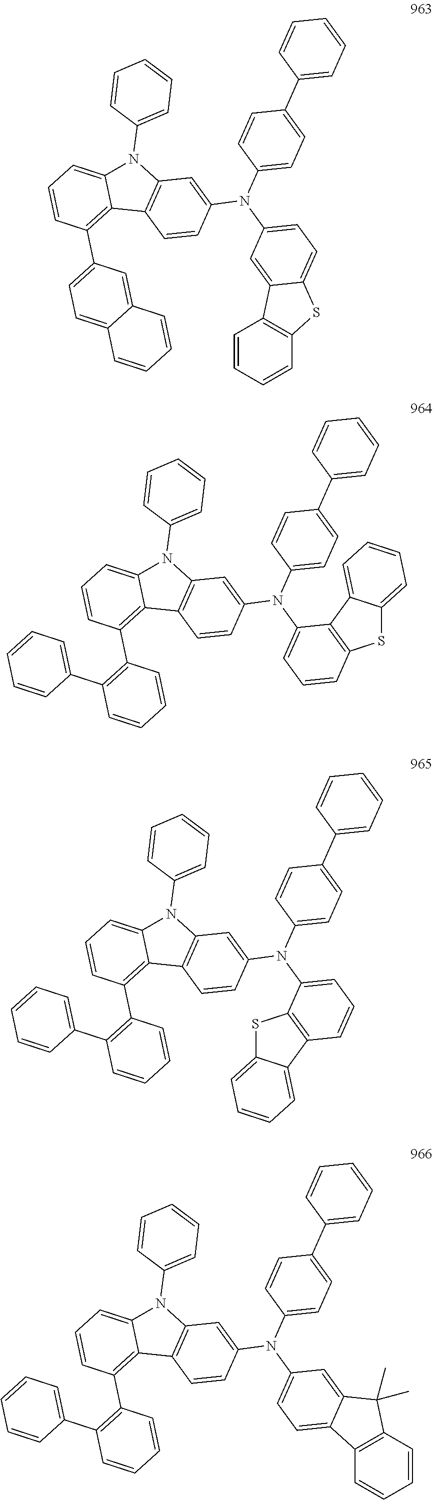 [SMILES: c1ccc(-c2ccc(N(c3ccc4sc5ccccc5c4c3)c3ccc4c5c(-c6ccc7ccccc7c6)cccc5n(-c5ccccc5)c4c3)cc2)cc1 ; c1ccc(-c2ccc(N(c3ccc4c5c(-c6ccccc6-c6ccccc6)cccc5n(-c5ccccc5)c4c3)c3cccc4sc5ccccc5c34)cc2)cc1 ; c1ccc(-c2ccc(N(c3ccc4c5c(-c6ccccc6-c6ccccc6)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6ccccc6-c6ccccc6)cccc5n(-c5ccccc5)c4c3)cc21]

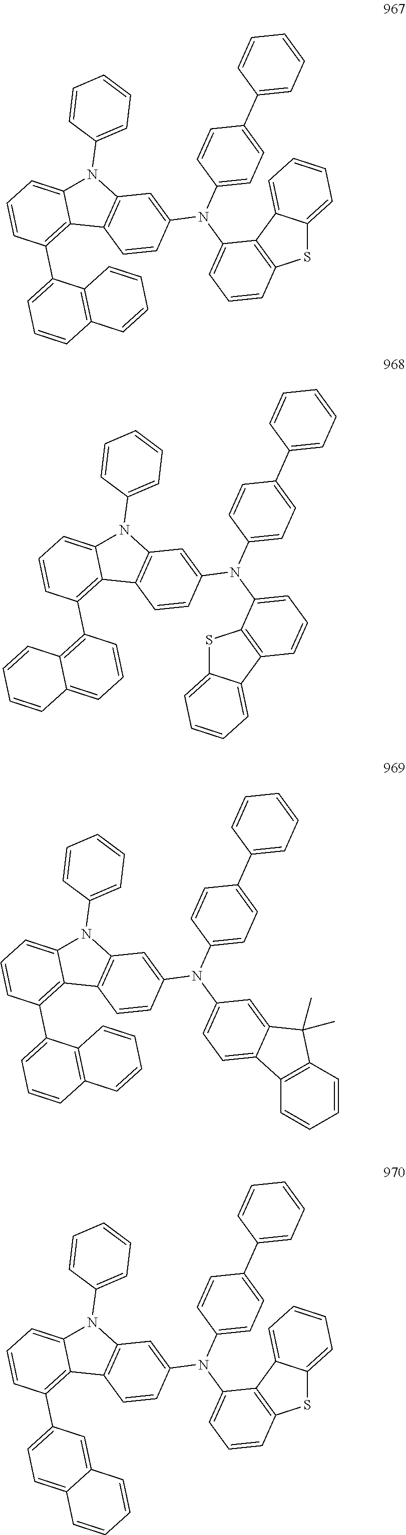 [SMILES: c1ccc(-c2ccc(N(c3ccc4c5c(-c6cccc7ccccc67)cccc5n(-c5ccccc5)c4c3)c3cccc4sc5ccccc5c34)cc2)cc1 ; c1ccc(-c2ccc(N(c3ccc4c5c(-c6cccc7ccccc67)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6cccc7ccccc67)cccc5n(-c5ccccc5)c4c3)cc21 ; c1ccc(-c2ccc(N(c3ccc4c5c(-c6ccc7ccccc7c6)cccc5n(-c5ccccc5)c4c3)c3cccc4sc5ccccc5c34)cc2)cc1]

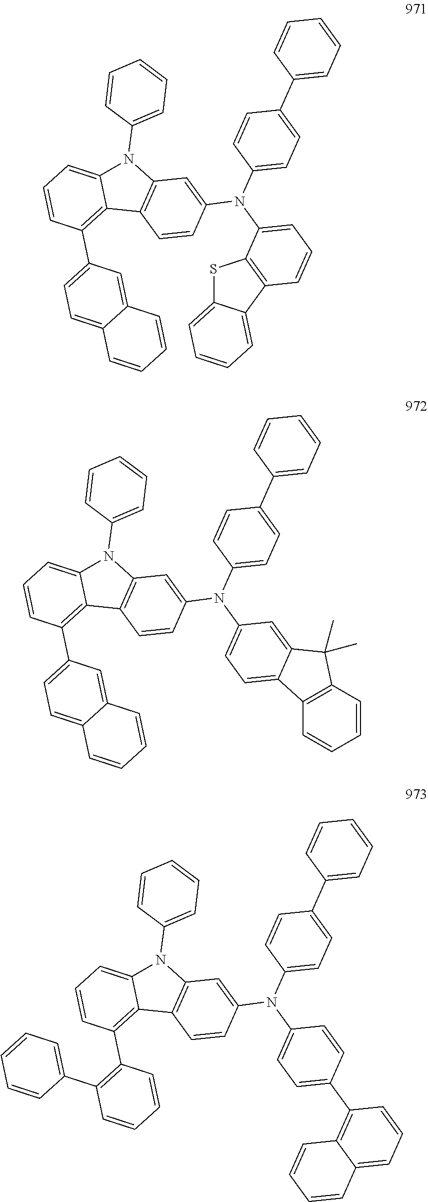 [SMILES: c1ccc(-c2ccc(N(c3ccc4c5c(-c6ccc7ccccc7c6)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6ccc7ccccc7c6)cccc5n(-c5ccccc5)c4c3)cc21 ; c1ccc(-c2ccc(N(c3ccc(-c4cccc5ccccc45)cc3)c3ccc4c5c(-c6ccccc6-c6ccccc6)cccc5n(-c5ccccc5)c4c3)cc2)cc1]

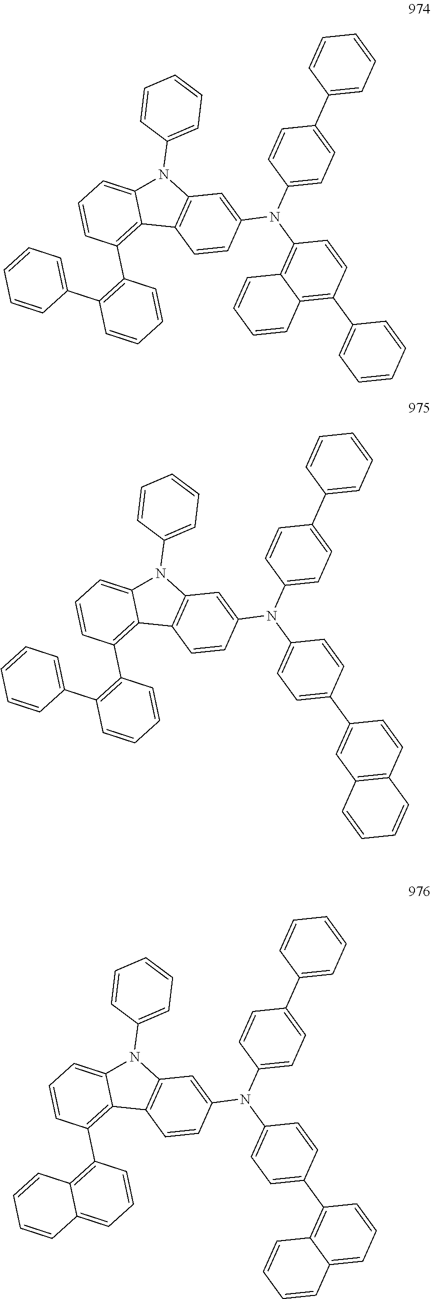 [SMILES: c1ccc(-c2ccc(N(c3ccc4c5c(-c6ccccc6-c6ccccc6)cccc5n(-c5ccccc5)c4c3)c3ccc(-c4ccccc4)c4ccccc34)cc2)cc1 ; c1ccc(-c2ccc(N(c3ccc(-c4ccc5ccccc5c4)cc3)c3ccc4c5c(-c6ccccc6-c6ccccc6)cccc5n(-c5ccccc5)c4c3)cc2)cc1 ; c1ccc(-c2ccc(N(c3ccc(-c4cccc5ccccc45)cc3)c3ccc4c5c(-c6cccc7ccccc67)cccc5n(-c5ccccc5)c4c3)cc2)cc1]

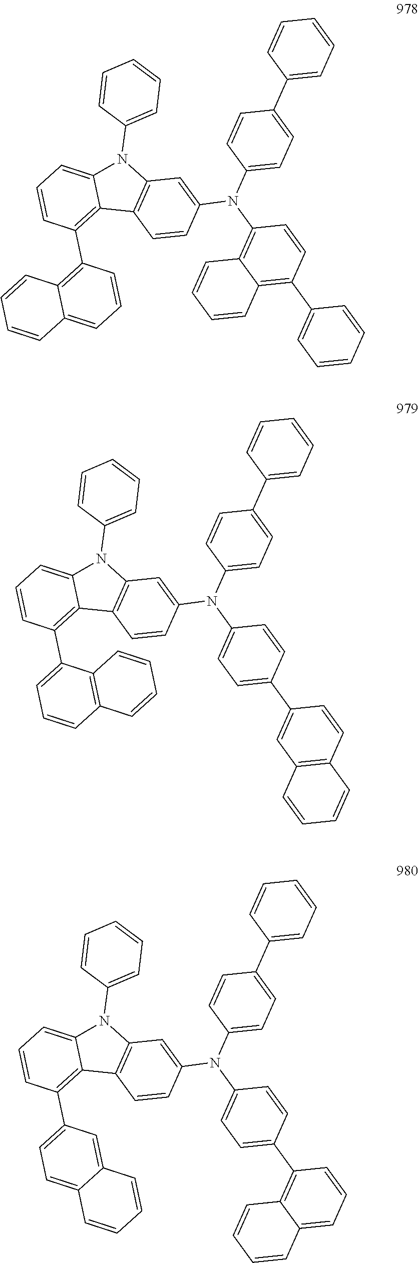 [SMILES: c1ccc(-c2ccc(N(c3ccc4c5c(-c6cccc7ccccc67)cccc5n(-c5ccccc5)c4c3)c3ccc(-c4ccccc4)c4ccccc34)cc2)cc1 ; c1ccc(-c2ccc(N(c3ccc(-c4ccc5ccccc5c4)cc3)c3ccc4c5c(-c6cccc7ccccc67)cccc5n(-c5ccccc5)c4c3)cc2)cc1 ; c1ccc(-c2ccc(N(c3ccc(-c4cccc5ccccc45)cc3)c3ccc4c5c(-c6ccc7ccccc7c6)cccc5n(-c5ccccc5)c4c3)cc2)cc1]

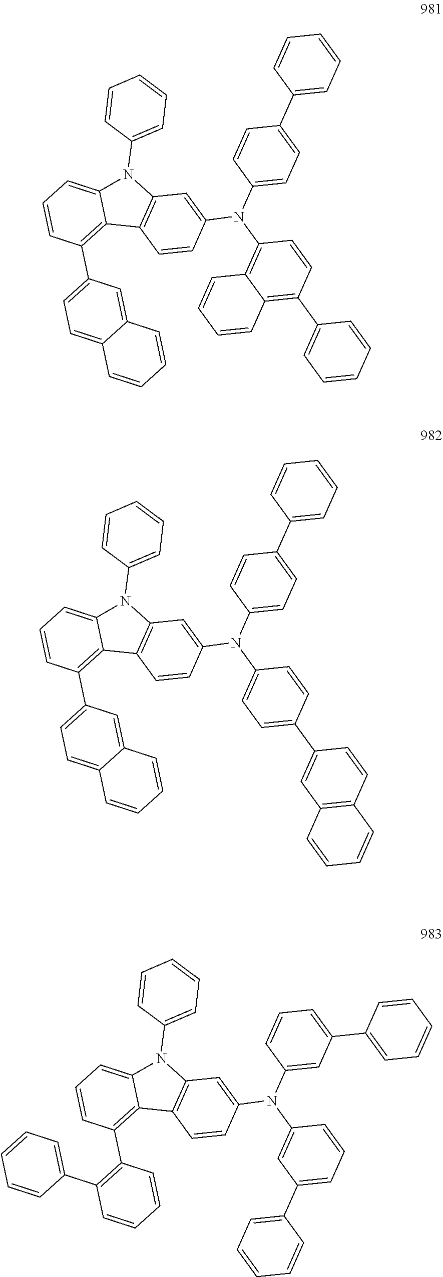 [SMILES: c1ccc(-c2ccc(N(c3ccc4c5c(-c6ccc7ccccc7c6)cccc5n(-c5ccccc5)c4c3)c3ccc(-c4ccccc4)c4ccccc34)cc2)cc1 ; c1ccc(-c2ccc(N(c3ccc(-c4ccc5ccccc5c4)cc3)c3ccc4c5c(-c6ccc7ccccc7c6)cccc5n(-c5ccccc5)c4c3)cc2)cc1 ; c1ccc(-c2cccc(N(c3cccc(-c4ccccc4)c3)c3ccc4c5c(-c6ccccc6-c6ccccc6)cccc5n(-c5ccccc5)c4c3)c2)cc1]

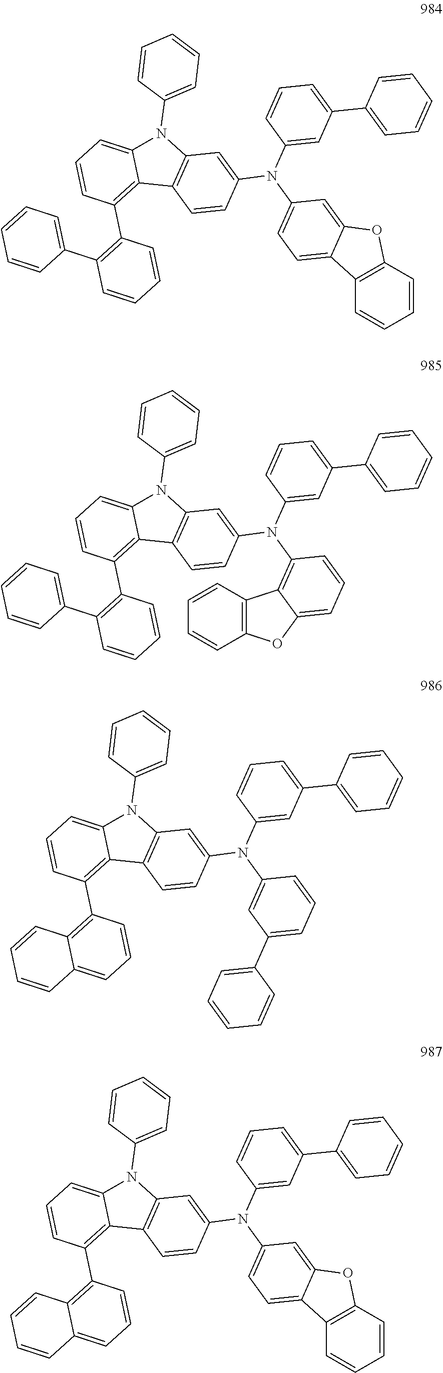 [SMILES: c1ccc(-c2cccc(N(c3ccc4c(c3)oc3ccccc34)c3ccc4c5c(-c6ccccc6-c6ccccc6)cccc5n(-c5ccccc5)c4c3)c2)cc1 ; c1ccc(-c2cccc(N(c3ccc4c5c(-c6ccccc6-c6ccccc6)cccc5n(-c5ccccc5)c4c3)c3cccc4oc5ccccc5c34)c2)cc1 ; c1ccc(-c2cccc(N(c3cccc(-c4ccccc4)c3)c3ccc4c5c(-c6cccc7ccccc67)cccc5n(-c5ccccc5)c4c3)c2)cc1 ; c1ccc(-c2cccc(N(c3ccc4c(c3)oc3ccccc34)c3ccc4c5c(-c6cccc7ccccc67)cccc5n(-c5ccccc5)c4c3)c2)cc1]

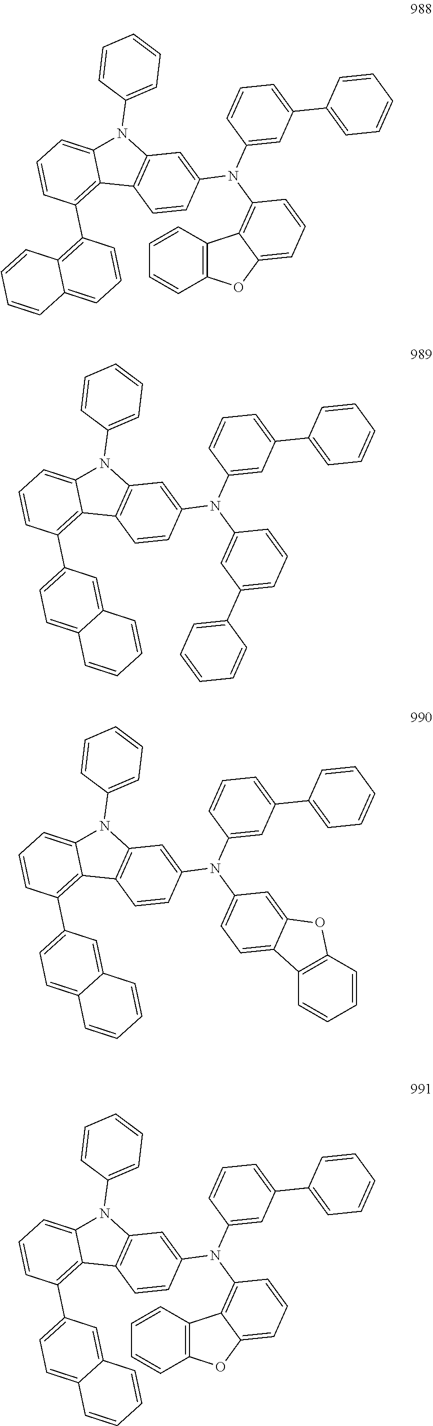 [SMILES: c1ccc(-c2cccc(N(c3ccc4c5c(-c6cccc7ccccc67)cccc5n(-c5ccccc5)c4c3)c3cccc4oc5ccccc5c34)c2)cc1 ; c1ccc(-c2cccc(N(c3cccc(-c4ccccc4)c3)c3ccc4c5c(-c6ccc7ccccc7c6)cccc5n(-c5ccccc5)c4c3)c2)cc1 ; c1ccc(-c2cccc(N(c3ccc4c(c3)oc3ccccc34)c3ccc4c5c(-c6ccc7ccccc7c6)cccc5n(-c5ccccc5)c4c3)c2)cc1 ; c1ccc(-c2cccc(N(c3ccc4c5c(-c6ccc7ccccc7c6)cccc5n(-c5ccccc5)c4c3)c3cccc4oc5ccccc5c34)c2)cc1]

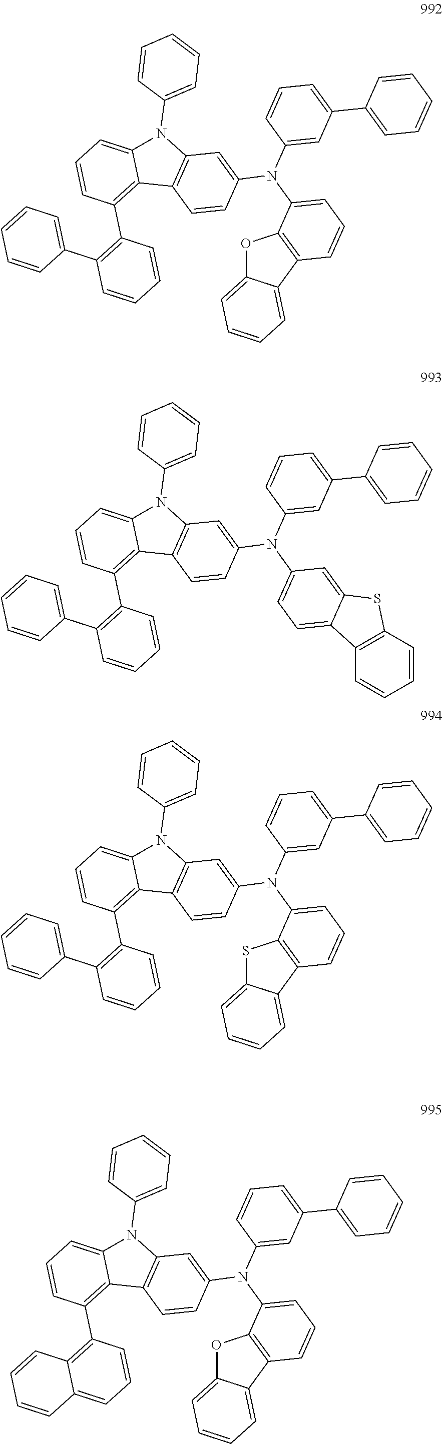 [SMILES: c1ccc(-c2cccc(N(c3ccc4c5c(-c6ccccc6-c6ccccc6)cccc5n(-c5ccccc5)c4c3)c3cccc4c3oc3ccccc34)c2)cc1 ; c1ccc(-c2cccc(N(c3ccc4c(c3)sc3ccccc34)c3ccc4c5c(-c6ccccc6-c6ccccc6)cccc5n(-c5ccccc5)c4c3)c2)cc1 ; c1ccc(-c2cccc(N(c3ccc4c5c(-c6ccccc6-c6ccccc6)cccc5n(-c5ccccc5)c4c3)c3cccc4c3sc3ccccc34)c2)cc1 ; c1ccc(-c2cccc(N(c3ccc4c5c(-c6cccc7ccccc67)cccc5n(-c5ccccc5)c4c3)c3cccc4c3oc3ccccc34)c2)cc1]

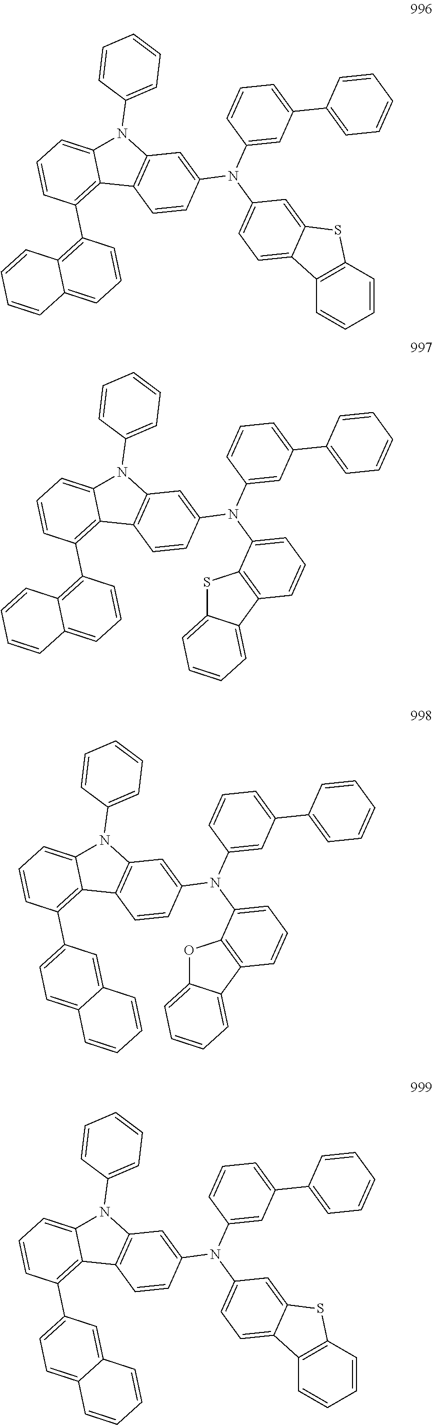 [SMILES: c1ccc(-c2cccc(N(c3ccc4c(c3)sc3ccccc34)c3ccc4c5c(-c6cccc7ccccc67)cccc5n(-c5ccccc5)c4c3)c2)cc1 ; c1ccc(-c2cccc(N(c3ccc4c5c(-c6cccc7ccccc67)cccc5n(-c5ccccc5)c4c3)c3cccc4c3sc3ccccc34)c2)cc1 ; c1ccc(-c2cccc(N(c3ccc4c5c(-c6ccc7ccccc7c6)cccc5n(-c5ccccc5)c4c3)c3cccc4c3oc3ccccc34)c2)cc1 ; c1ccc(-c2cccc(N(c3ccc4c(c3)sc3ccccc34)c3ccc4c5c(-c6ccc7ccccc7c6)cccc5n(-c5ccccc5)c4c3)c2)cc1]

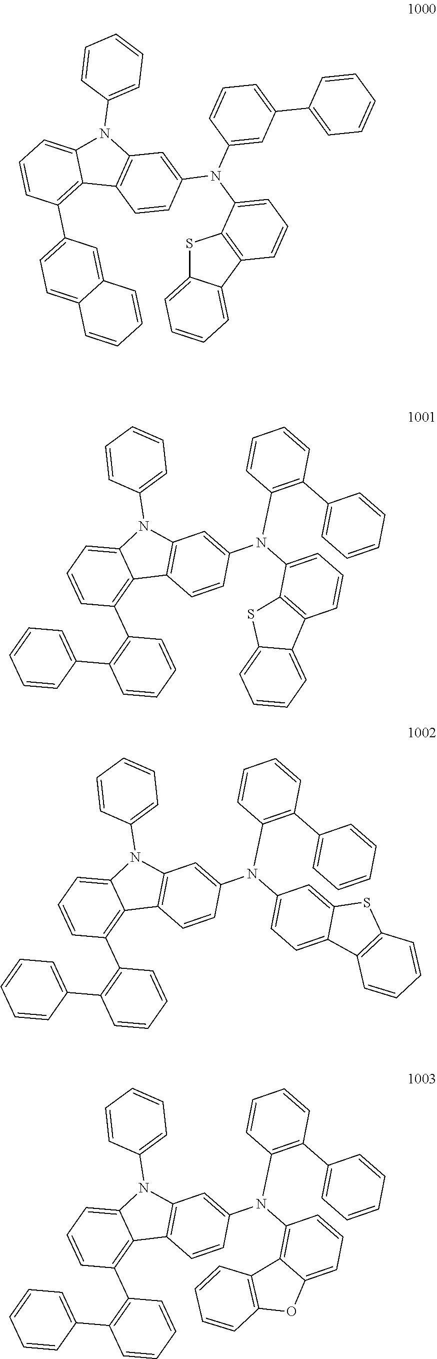 [SMILES: c1ccc(-c2cccc(N(c3ccc4c5c(-c6ccc7ccccc7c6)cccc5n(-c5ccccc5)c4c3)c3cccc4c3sc3ccccc34)c2)cc1 ; c1ccc(-c2ccccc2-c2cccc3c2c2ccc(N(c4ccccc4-c4ccccc4)c4cccc5c4sc4ccccc45)cc2n3-c2ccccc2)cc1 ; c1ccc(-c2ccccc2-c2cccc3c2c2ccc(N(c4ccc5c(c4)sc4ccccc45)c4ccccc4-c4ccccc4)cc2n3-c2ccccc2)cc1 ; c1ccc(-c2ccccc2-c2cccc3c2c2ccc(N(c4ccccc4-c4ccccc4)c4cccc5oc6ccccc6c45)cc2n3-c2ccccc2)cc1]

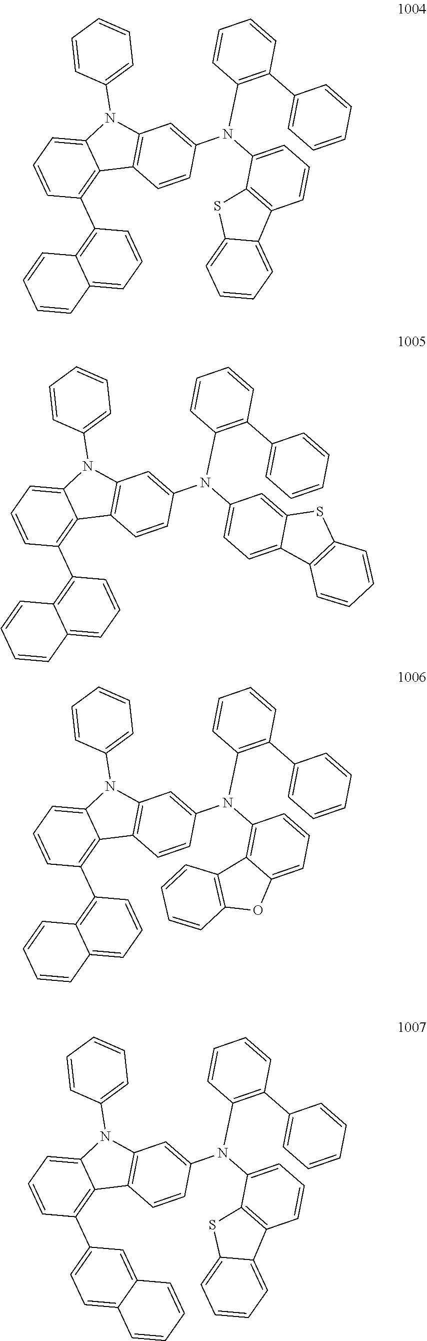 [SMILES: c1ccc(-c2ccccc2N(c2ccc3c4c(-c5cccc6ccccc56)cccc4n(-c4ccccc4)c3c2)c2cccc3c2sc2ccccc23)cc1 ; c1ccc(-c2ccccc2N(c2ccc3c(c2)sc2ccccc23)c2ccc3c4c(-c5cccc6ccccc56)cccc4n(-c4ccccc4)c3c2)cc1 ; c1ccc(-c2ccccc2N(c2ccc3c4c(-c5cccc6ccccc56)cccc4n(-c4ccccc4)c3c2)c2cccc3oc4ccccc4c23)cc1 ; c1ccc(-c2ccccc2N(c2ccc3c4c(-c5ccc6ccccc6c5)cccc4n(-c4ccccc4)c3c2)c2cccc3c2sc2ccccc23)cc1]

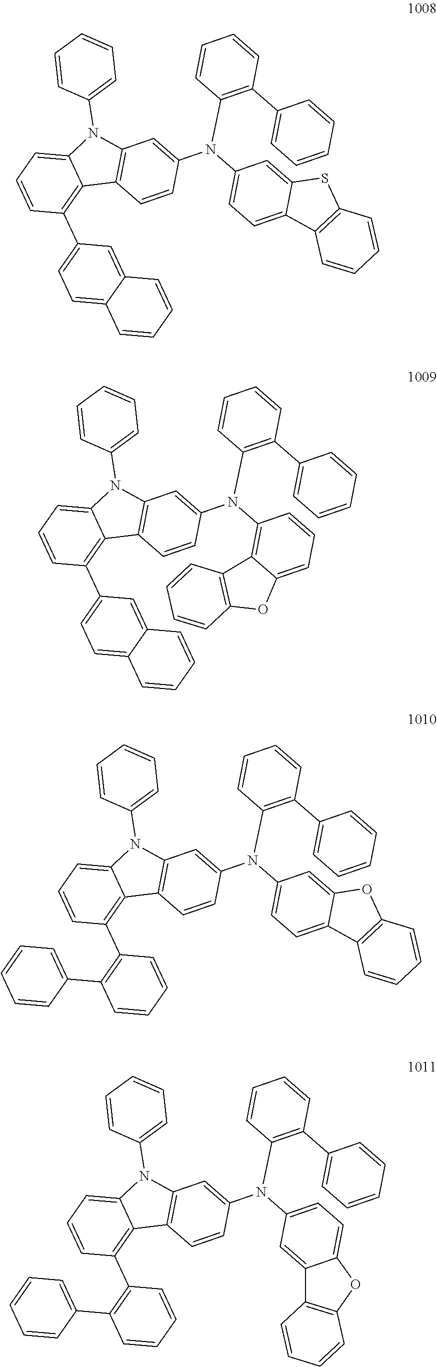 [SMILES: c1ccc(-c2ccccc2N(c2ccc3c(c2)sc2ccccc23)c2ccc3c4c(-c5ccc6ccccc6c5)cccc4n(-c4ccccc4)c3c2)cc1 ; c1ccc(-c2ccccc2N(c2ccc3c4c(-c5ccc6ccccc6c5)cccc4n(-c4ccccc4)c3c2)c2cccc3oc4ccccc4c23)cc1 ; c1ccc(-c2ccccc2-c2cccc3c2c2ccc(N(c4ccc5c(c4)oc4ccccc45)c4ccccc4-c4ccccc4)cc2n3-c2ccccc2)cc1 ; c1ccc(-c2ccccc2-c2cccc3c2c2ccc(N(c4ccc5oc6ccccc6c5c4)c4ccccc4-c4ccccc4)cc2n3-c2ccccc2)cc1]

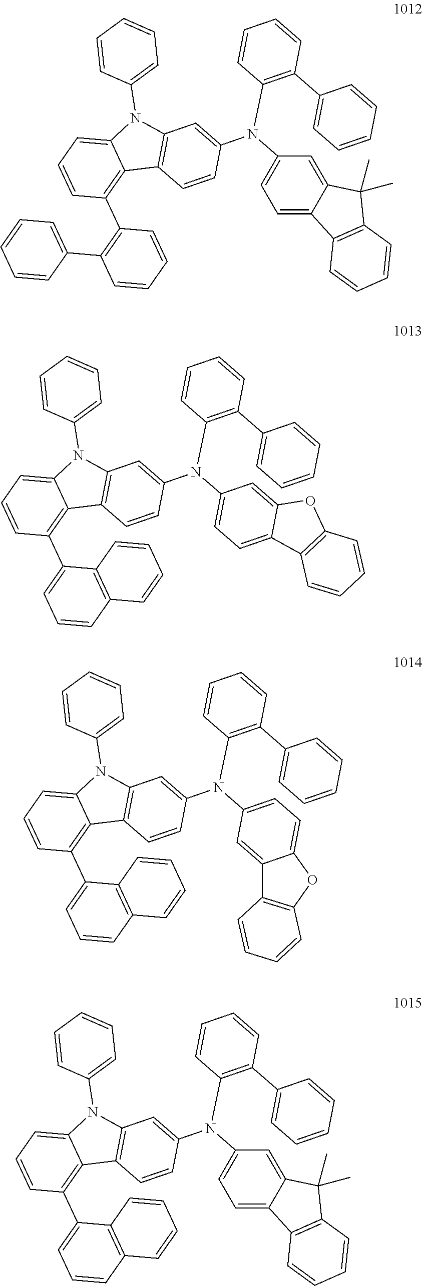

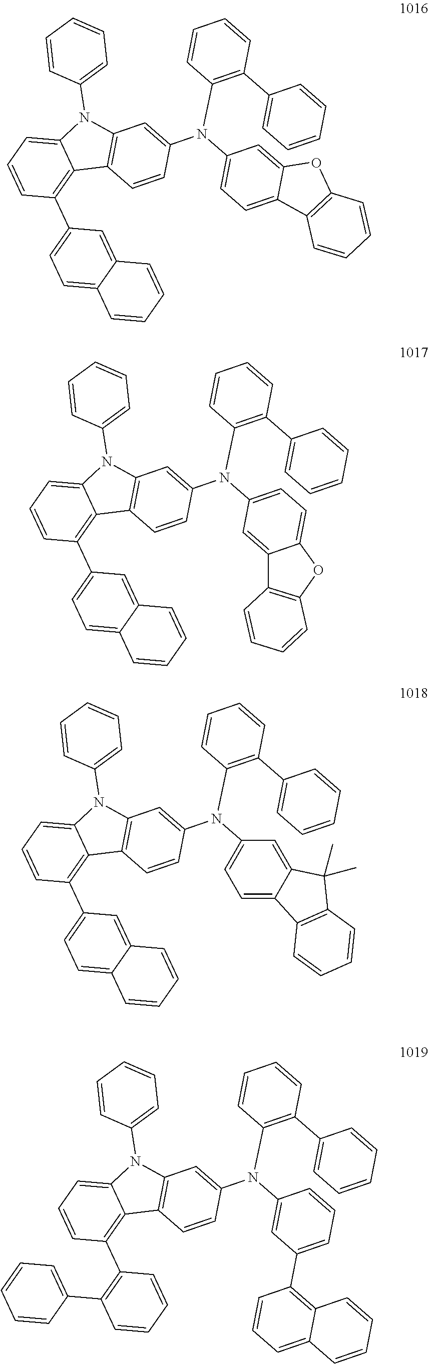

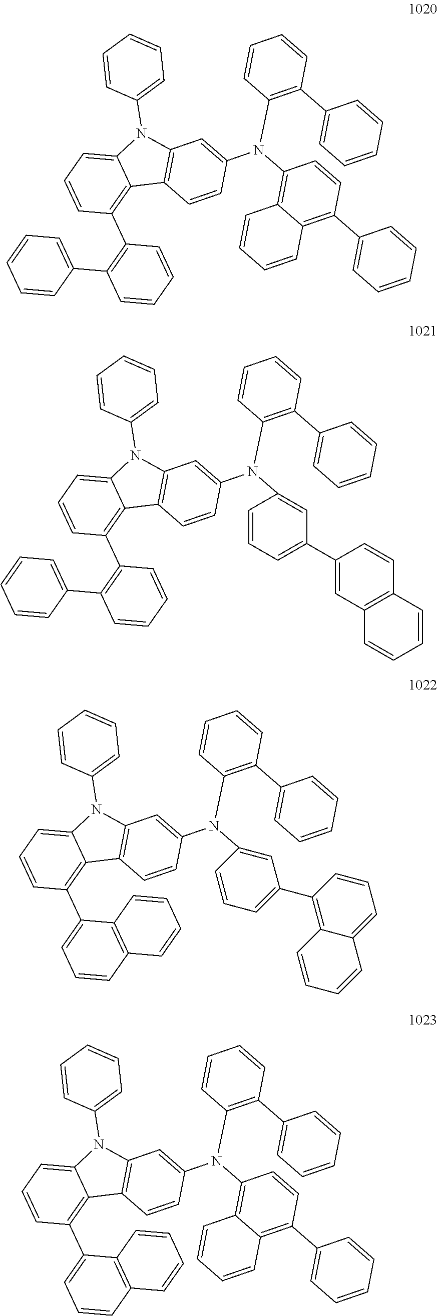 [SMILES: c1ccc(-c2ccccc2-c2cccc3c2c2ccc(N(c4ccccc4-c4ccccc4)c4ccc(-c5ccccc5)c5ccccc45)cc2n3-c2ccccc2)cc1 ; c1ccc(-c2ccccc2-c2cccc3c2c2ccc(N(c4cccc(-c5ccc6ccccc6c5)c4)c4ccccc4-c4ccccc4)cc2n3-c2ccccc2)cc1 ; c1ccc(-c2ccccc2N(c2cccc(-c3cccc4ccccc34)c2)c2ccc3c4c(-c5cccc6ccccc56)cccc4n(-c4ccccc4)c3c2)cc1 ; c1ccc(-c2ccccc2N(c2ccc3c4c(-c5cccc6ccccc56)cccc4n(-c4ccccc4)c3c2)c2ccc(-c3ccccc3)c3ccccc23)cc1]

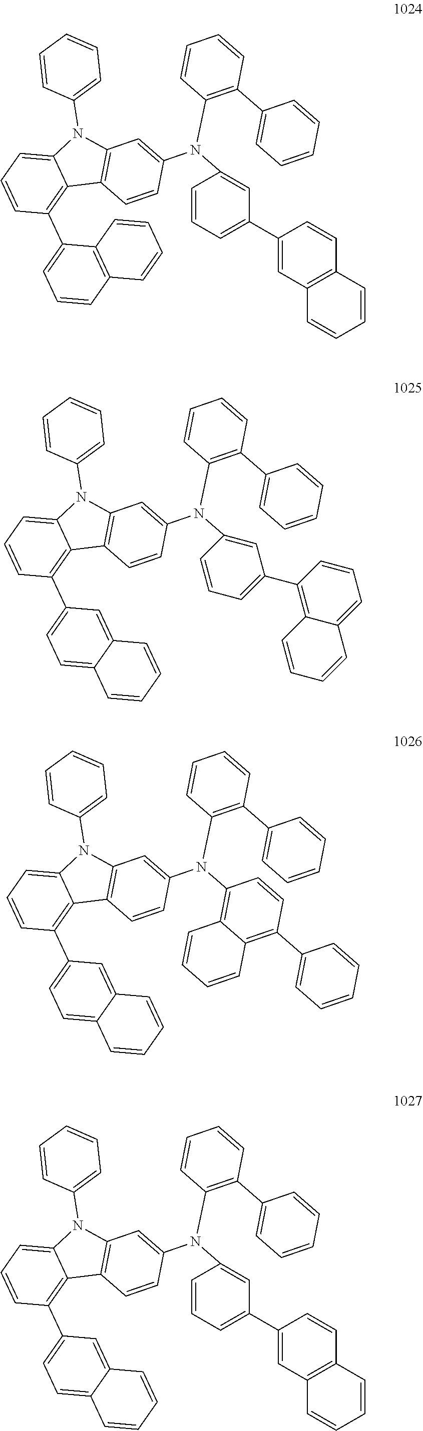 [SMILES: c1ccc(-c2ccccc2N(c2cccc(-c3ccc4ccccc4c3)c2)c2ccc3c4c(-c5cccc6ccccc56)cccc4n(-c4ccccc4)c3c2)cc1 ; c1ccc(-c2ccccc2N(c2cccc(-c3cccc4ccccc34)c2)c2ccc3c4c(-c5ccc6ccccc6c5)cccc4n(-c4ccccc4)c3c2)cc1 ; c1ccc(-c2ccccc2N(c2ccc3c4c(-c5ccc6ccccc6c5)cccc4n(-c4ccccc4)c3c2)c2ccc(-c3ccccc3)c3ccccc23)cc1 ; c1ccc(-c2ccccc2N(c2cccc(-c3ccc4ccccc4c3)c2)c2ccc3c4c(-c5ccc6ccccc6c5)cccc4n(-c4ccccc4)c3c2)cc1]

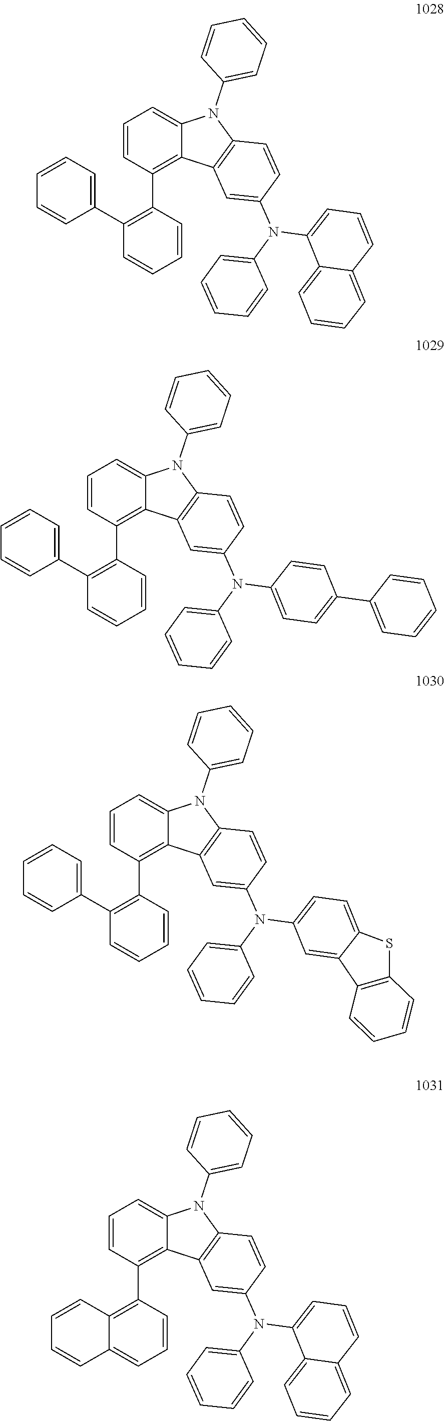

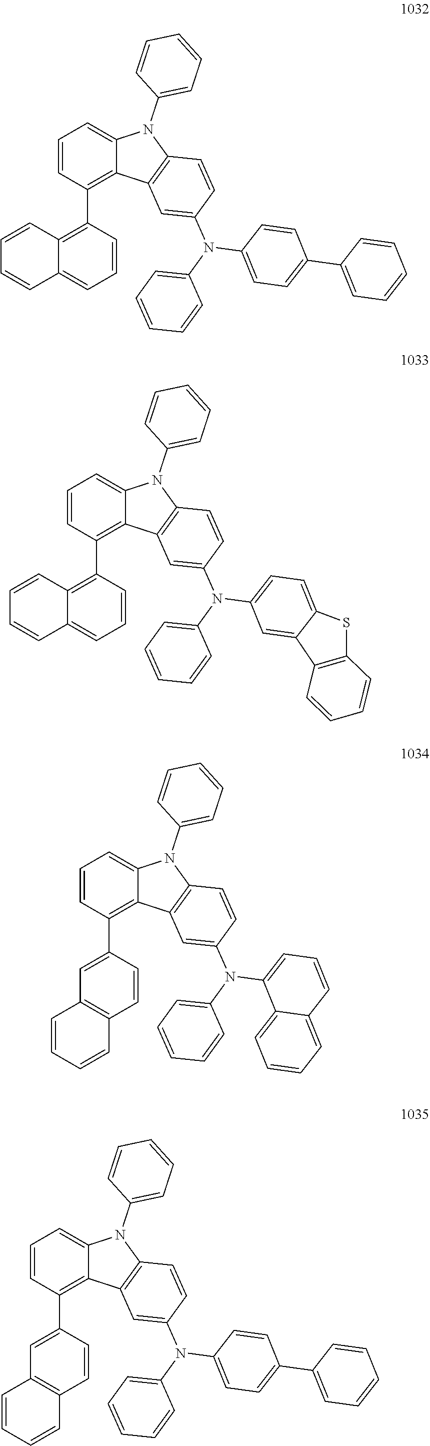 [SMILES: c1ccc(-c2ccc(N(c3ccccc3)c3ccc4c(c3)c3c(-c5cccc6ccccc56)cccc3n4-c3ccccc3)cc2)cc1 ; c1ccc(N(c2ccc3sc4ccccc4c3c2)c2ccc3c(c2)c2c(-c4cccc5ccccc45)cccc2n3-c2ccccc2)cc1 ; c1ccc(N(c2ccc3c(c2)c2c(-c4ccc5ccccc5c4)cccc2n3-c2ccccc2)c2cccc3ccccc23)cc1 ; c1ccc(-c2ccc(N(c3ccccc3)c3ccc4c(c3)c3c(-c5ccc6ccccc6c5)cccc3n4-c3ccccc3)cc2)cc1]

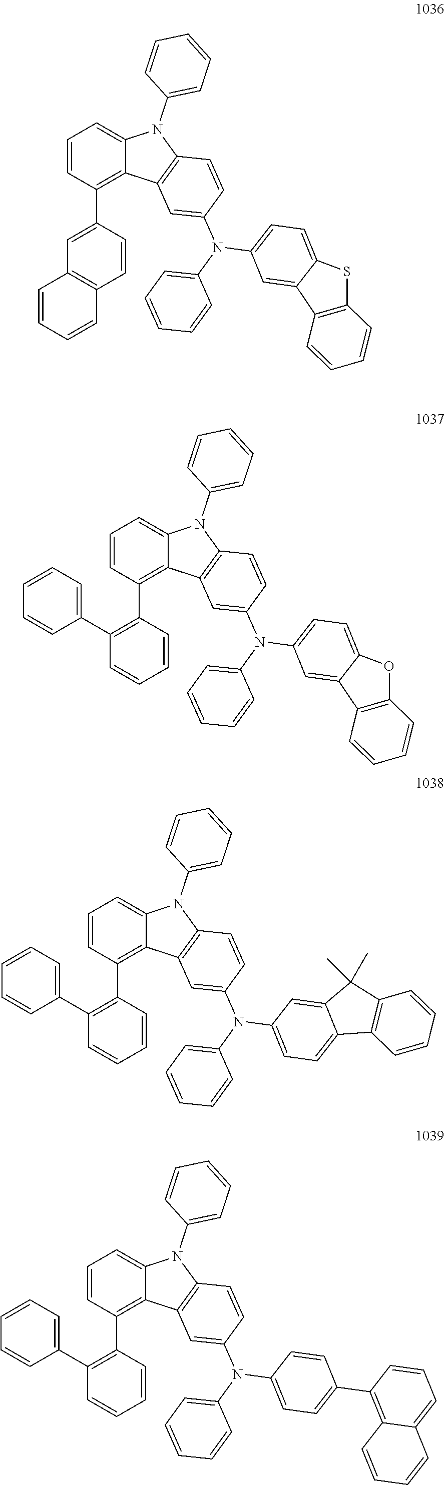 [SMILES: c1ccc(N(c2ccc3sc4ccccc4c3c2)c2ccc3c(c2)c2c(-c4ccc5ccccc5c4)cccc2n3-c2ccccc2)cc1 ; c1ccc(-c2ccccc2-c2cccc3c2c2cc(N(c4ccccc4)c4ccc5oc6ccccc6c5c4)ccc2n3-c2ccccc2)cc1 ; CC1(C)c2ccccc2-c2ccc(N(c3ccccc3)c3ccc4c(c3)c3c(-c5ccccc5-c5ccccc5)cccc3n4-c3ccccc3)cc21 ; c1ccc(-c2ccccc2-c2cccc3c2c2cc(N(c4ccccc4)c4ccc(-c5cccc6ccccc56)cc4)ccc2n3-c2ccccc2)cc1]

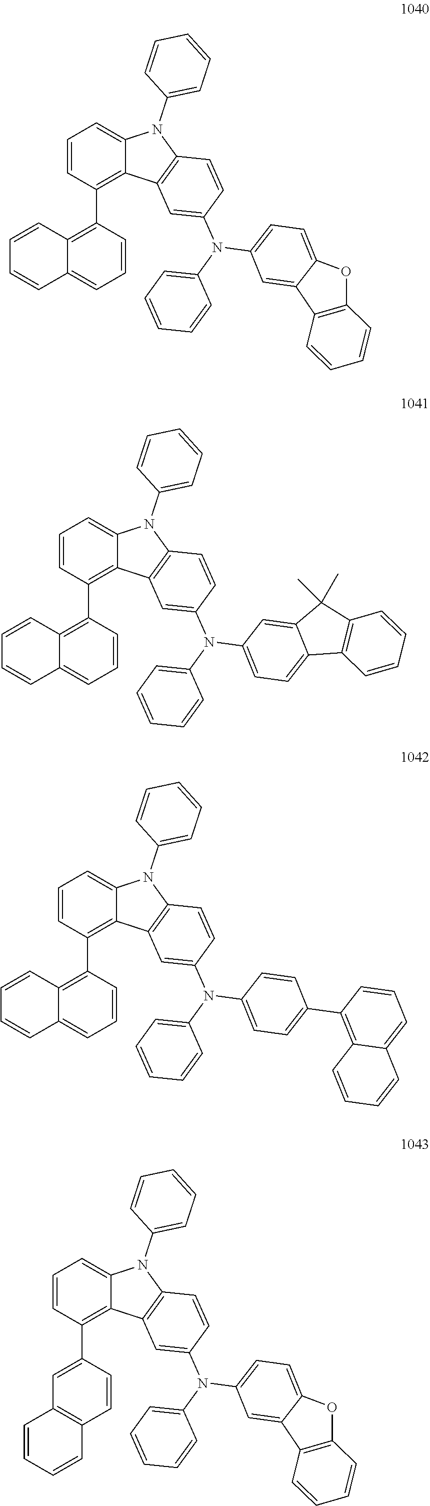 [SMILES: c1ccc(N(c2ccc3oc4ccccc4c3c2)c2ccc3c(c2)c2c(-c4cccc5ccccc45)cccc2n3-c2ccccc2)cc1 ; CC1(C)c2ccccc2-c2ccc(N(c3ccccc3)c3ccc4c(c3)c3c(-c5cccc6ccccc56)cccc3n4-c3ccccc3)cc21 ; c1ccc(N(c2ccc(-c3cccc4ccccc34)cc2)c2ccc3c(c2)c2c(-c4cccc5ccccc45)cccc2n3-c2ccccc2)cc1 ; c1ccc(N(c2ccc3oc4ccccc4c3c2)c2ccc3c(c2)c2c(-c4ccc5ccccc5c4)cccc2n3-c2ccccc2)cc1]

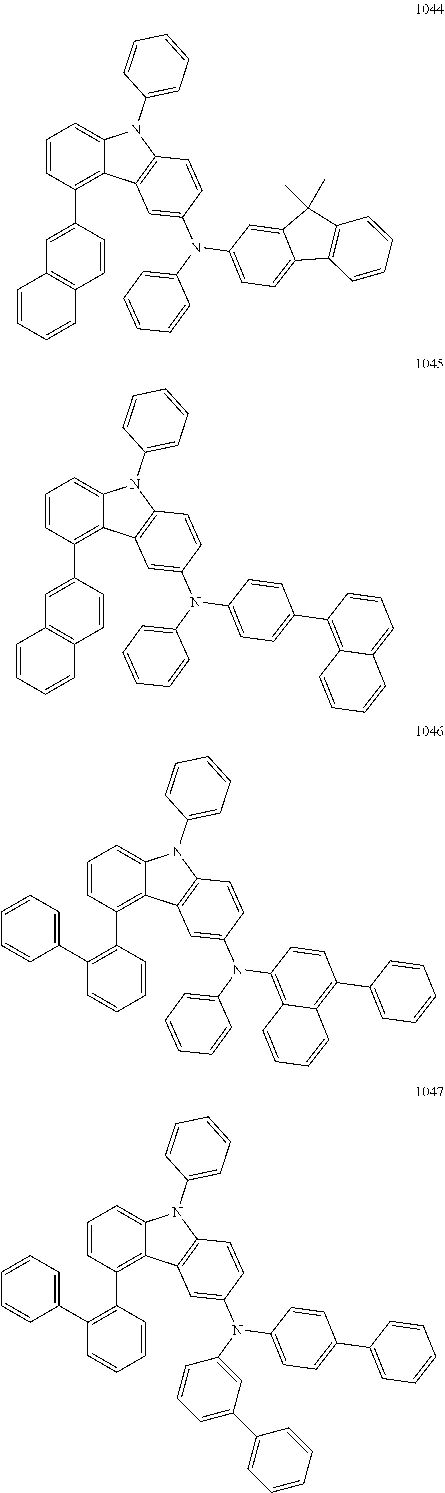 [SMILES: CC1(C)c2ccccc2-c2ccc(N(c3ccccc3)c3ccc4c(c3)c3c(-c5ccc6ccccc6c5)cccc3n4-c3ccccc3)cc21 ; c1ccc(N(c2ccc(-c3cccc4ccccc34)cc2)c2ccc3c(c2)c2c(-c4ccc5ccccc5c4)cccc2n3-c2ccccc2)cc1 ; c1ccc(-c2ccccc2-c2cccc3c2c2cc(N(c4ccccc4)c4ccc(-c5ccccc5)c5ccccc45)ccc2n3-c2ccccc2)cc1 ; c1ccc(-c2ccc(N(c3cccc(-c4ccccc4)c3)c3ccc4c(c3)c3c(-c5ccccc5-c5ccccc5)cccc3n4-c3ccccc3)cc2)cc1]

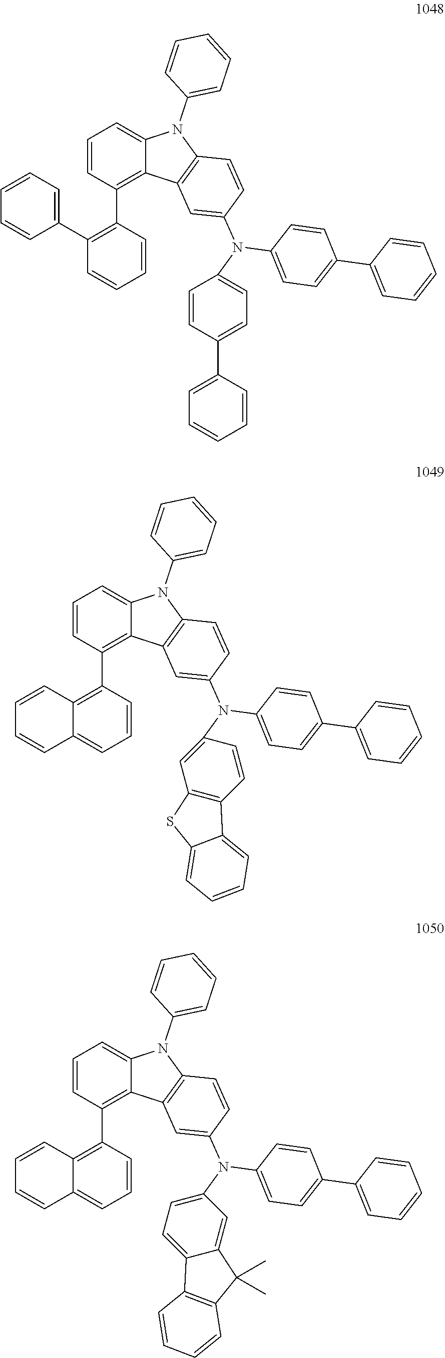 [SMILES: c1ccc(-c2ccc(N(c3ccc(-c4ccccc4)cc3)c3ccc4c(c3)c3c(-c5ccccc5-c5ccccc5)cccc3n4-c3ccccc3)cc2)cc1 ; c1ccc(-c2ccc(N(c3ccc4c(c3)sc3ccccc34)c3ccc4c(c3)c3c(-c5cccc6ccccc56)cccc3n4-c3ccccc3)cc2)cc1 ; CC1(C)c2ccccc2-c2ccc(N(c3ccc(-c4ccccc4)cc3)c3ccc4c(c3)c3c(-c5cccc6ccccc56)cccc3n4-c3ccccc3)cc21]

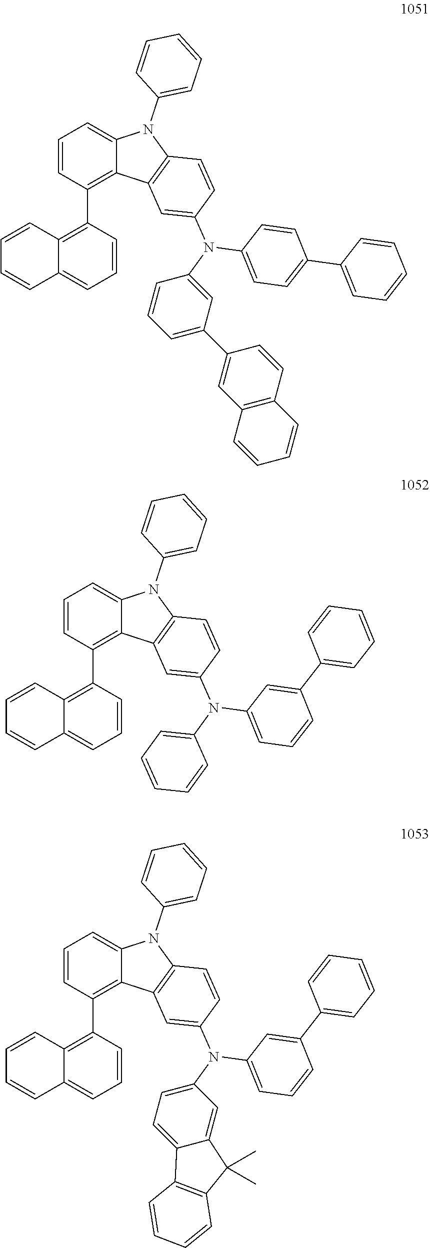 [SMILES: c1ccc(-c2ccc(N(c3cccc(-c4ccc5ccccc5c4)c3)c3ccc4c(c3)c3c(-c5cccc6ccccc56)cccc3n4-c3ccccc3)cc2)cc1 ; c1ccc(-c2cccc(N(c3ccccc3)c3ccc4c(c3)c3c(-c5cccc6ccccc56)cccc3n4-c3ccccc3)c2)cc1 ; CC1(C)c2ccccc2-c2ccc(N(c3cccc(-c4ccccc4)c3)c3ccc4c(c3)c3c(-c5cccc6ccccc56)cccc3n4-c3ccccc3)cc21]

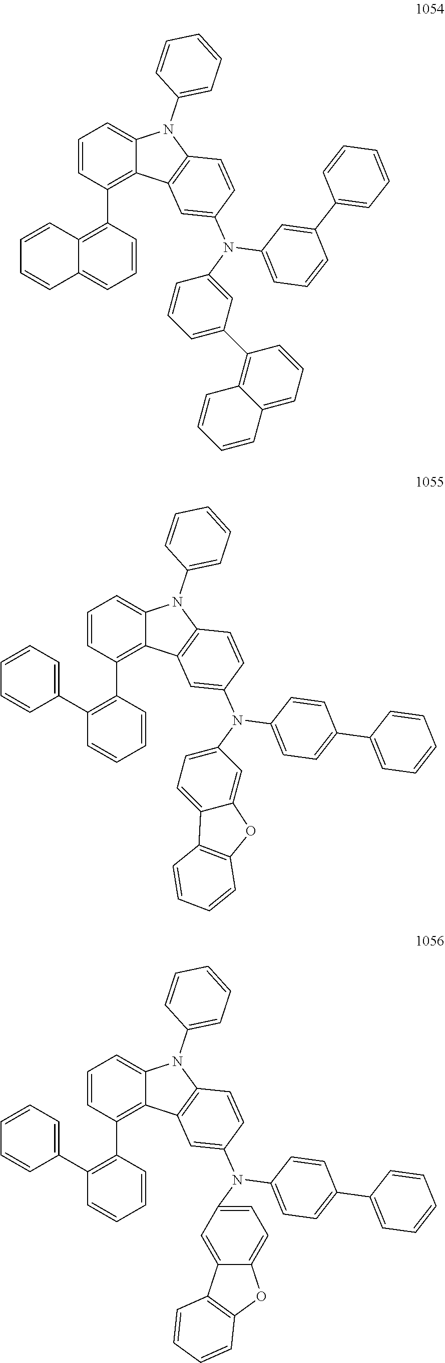 [SMILES: c1ccc(-c2cccc(N(c3cccc(-c4cccc5ccccc45)c3)c3ccc4c(c3)c3c(-c5cccc6ccccc56)cccc3n4-c3ccccc3)c2)cc1 ; c1ccc(-c2ccc(N(c3ccc4c(c3)oc3ccccc34)c3ccc4c(c3)c3c(-c5ccccc5-c5ccccc5)cccc3n4-c3ccccc3)cc2)cc1 ; c1ccc(-c2ccc(N(c3ccc4oc5ccccc5c4c3)c3ccc4c(c3)c3c(-c5ccccc5-c5ccccc5)cccc3n4-c3ccccc3)cc2)cc1]

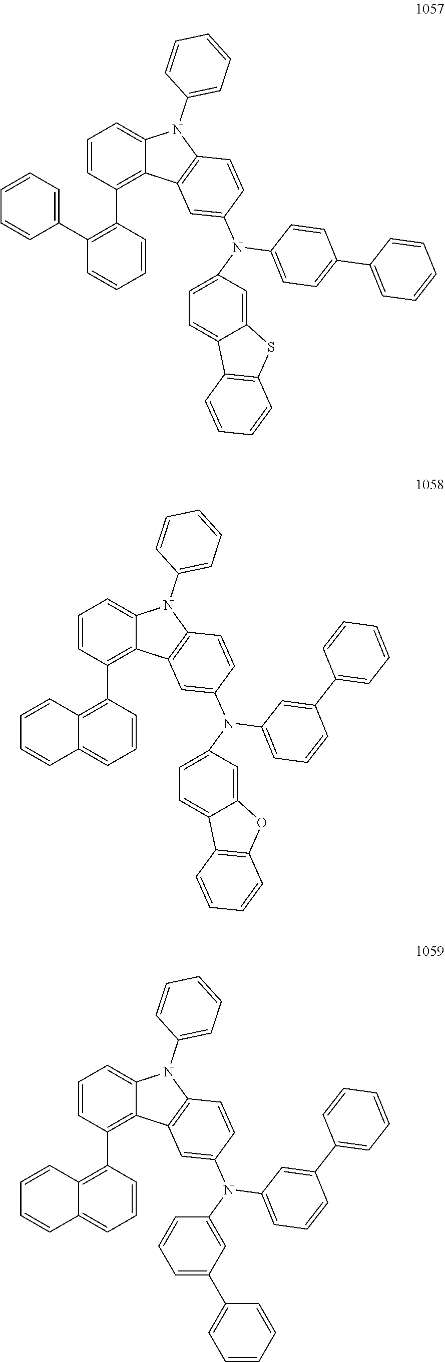 [SMILES: c1ccc(-c2ccc(N(c3ccc4c(c3)sc3ccccc34)c3ccc4c(c3)c3c(-c5ccccc5-c5ccccc5)cccc3n4-c3ccccc3)cc2)cc1 ; c1ccc(-c2cccc(N(c3ccc4c(c3)oc3ccccc34)c3ccc4c(c3)c3c(-c5cccc6ccccc56)cccc3n4-c3ccccc3)c2)cc1 ; c1ccc(-c2cccc(N(c3cccc(-c4ccccc4)c3)c3ccc4c(c3)c3c(-c5cccc6ccccc56)cccc3n4-c3ccccc3)c2)cc1]

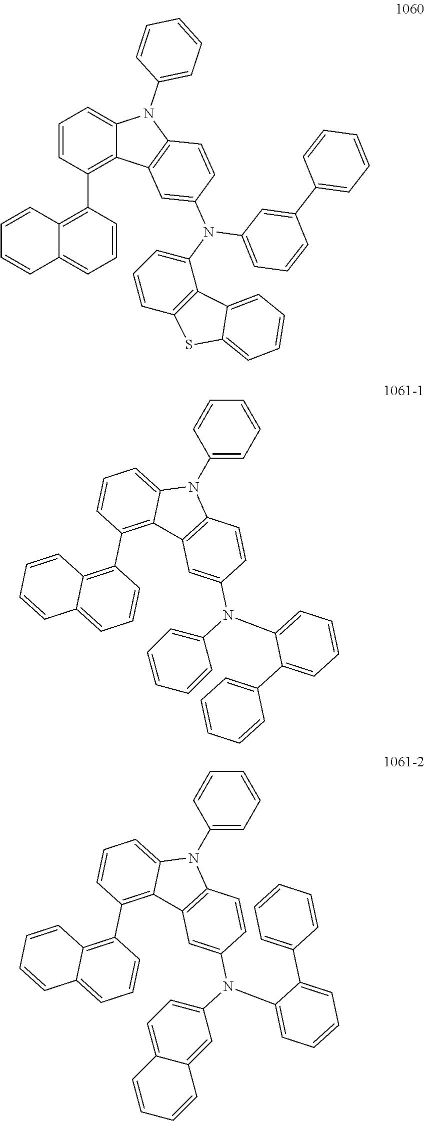

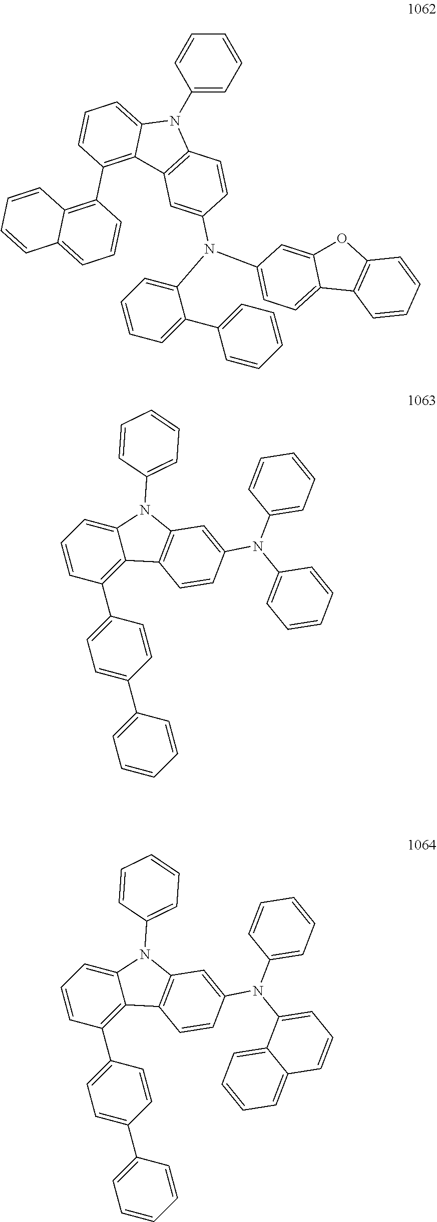 [SMILES: c1ccc(-c2ccccc2N(c2ccc3c(c2)oc2ccccc23)c2ccc3c(c2)c2c(-c4cccc5ccccc45)cccc2n3-c2ccccc2)cc1 ; c1ccc(-c2ccc(-c3cccc4c3c3ccc(N(c5ccccc5)c5ccccc5)cc3n4-c3ccccc3)cc2)cc1 ; c1ccc(-c2ccc(-c3cccc4c3c3ccc(N(c5ccccc5)c5cccc6ccccc56)cc3n4-c3ccccc3)cc2)cc1]

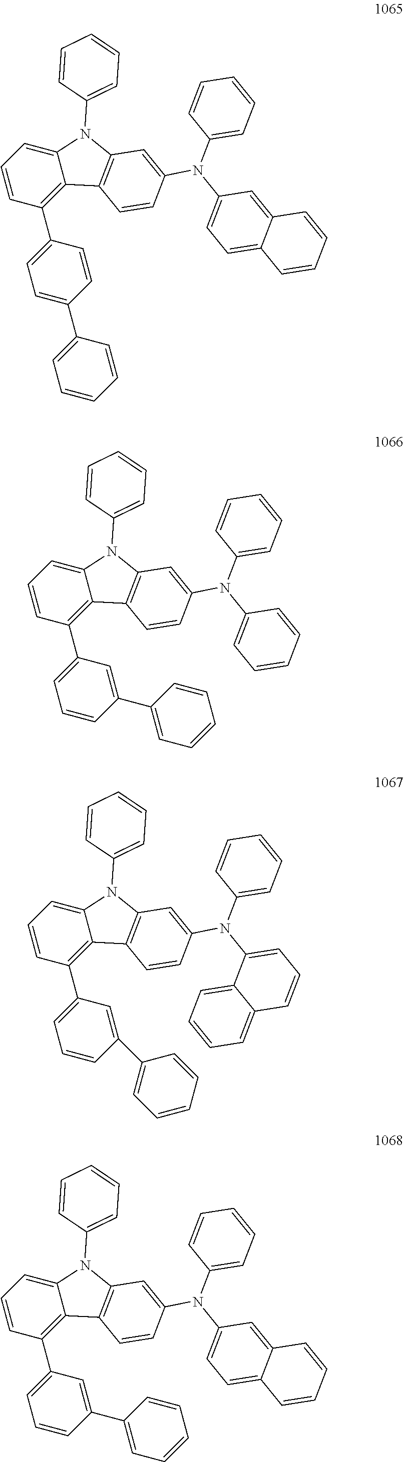 [SMILES: c1ccc(-c2ccc(-c3cccc4c3c3ccc(N(c5ccccc5)c5ccc6ccccc6c5)cc3n4-c3ccccc3)cc2)cc1 ; c1ccc(-c2cccc(-c3cccc4c3c3ccc(N(c5ccccc5)c5ccccc5)cc3n4-c3ccccc3)c2)cc1 ; c1ccc(-c2cccc(-c3cccc4c3c3ccc(N(c5ccccc5)c5cccc6ccccc56)cc3n4-c3ccccc3)c2)cc1 ; c1ccc(-c2cccc(-c3cccc4c3c3ccc(N(c5ccccc5)c5ccc6ccccc6c5)cc3n4-c3ccccc3)c2)cc1]

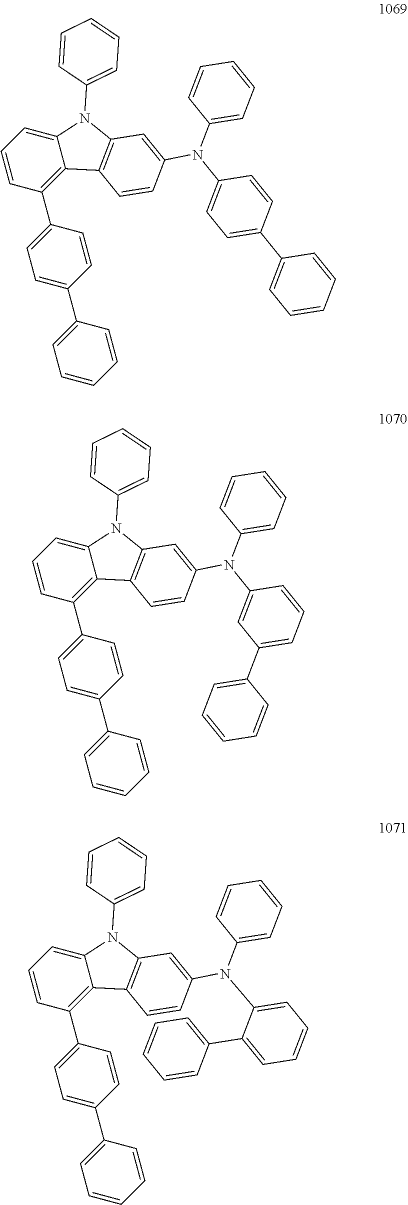

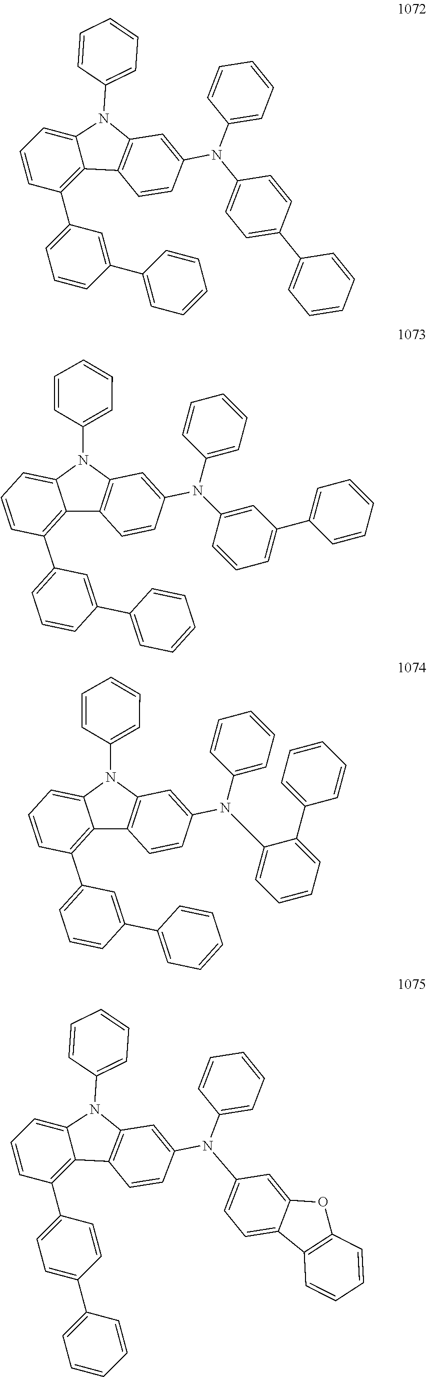

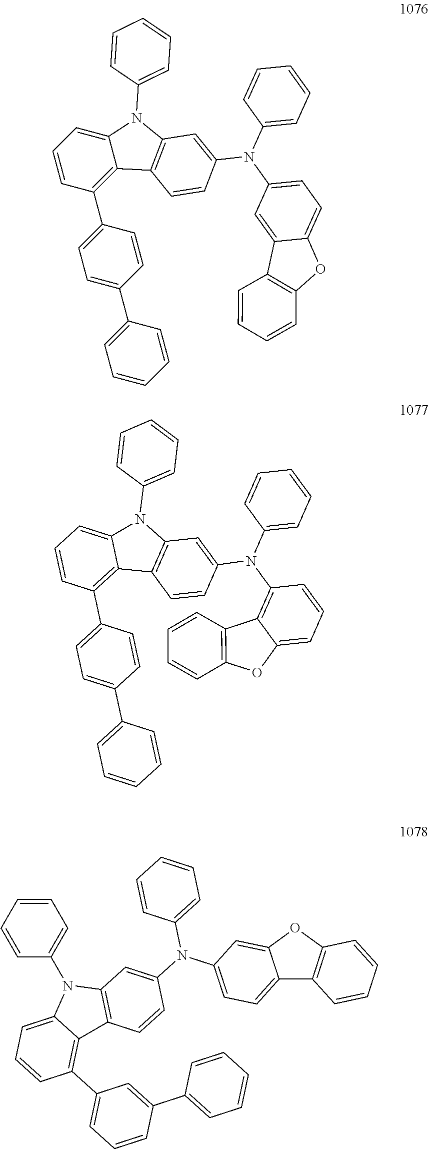

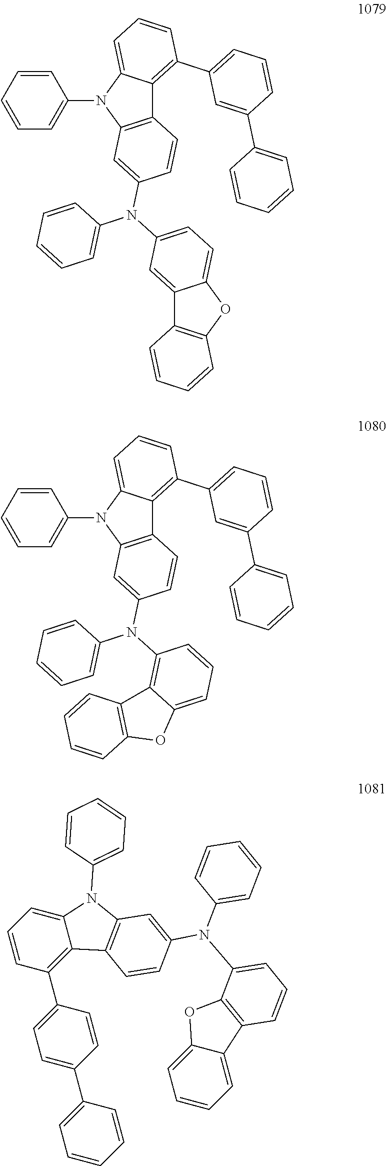

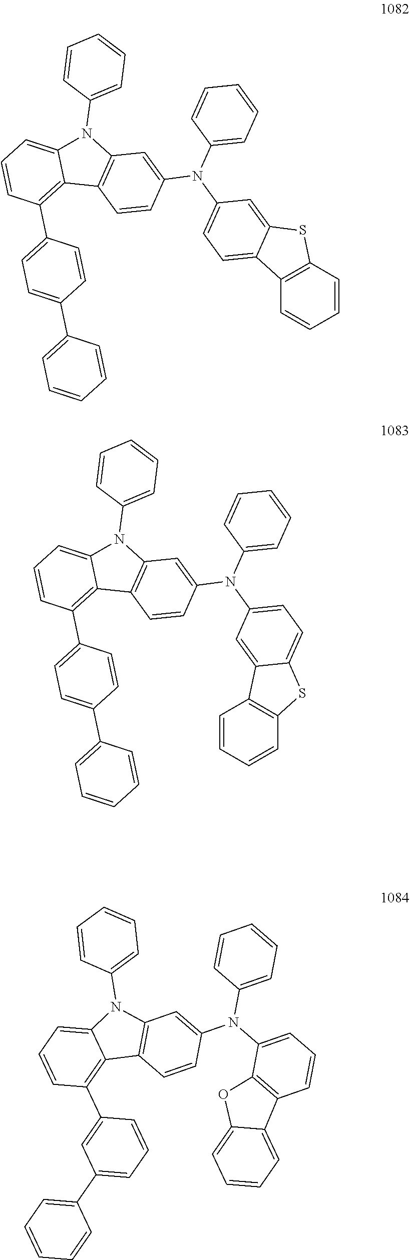 [SMILES: c1ccc(-c2ccc(-c3cccc4c3c3ccc(N(c5ccccc5)c5ccc6c(c5)sc5ccccc56)cc3n4-c3ccccc3)cc2)cc1 ; c1ccc(-c2ccc(-c3cccc4c3c3ccc(N(c5ccccc5)c5ccc6sc7ccccc7c6c5)cc3n4-c3ccccc3)cc2)cc1 ; c1ccc(-c2cccc(-c3cccc4c3c3ccc(N(c5ccccc5)c5cccc6c5oc5ccccc56)cc3n4-c3ccccc3)c2)cc1]

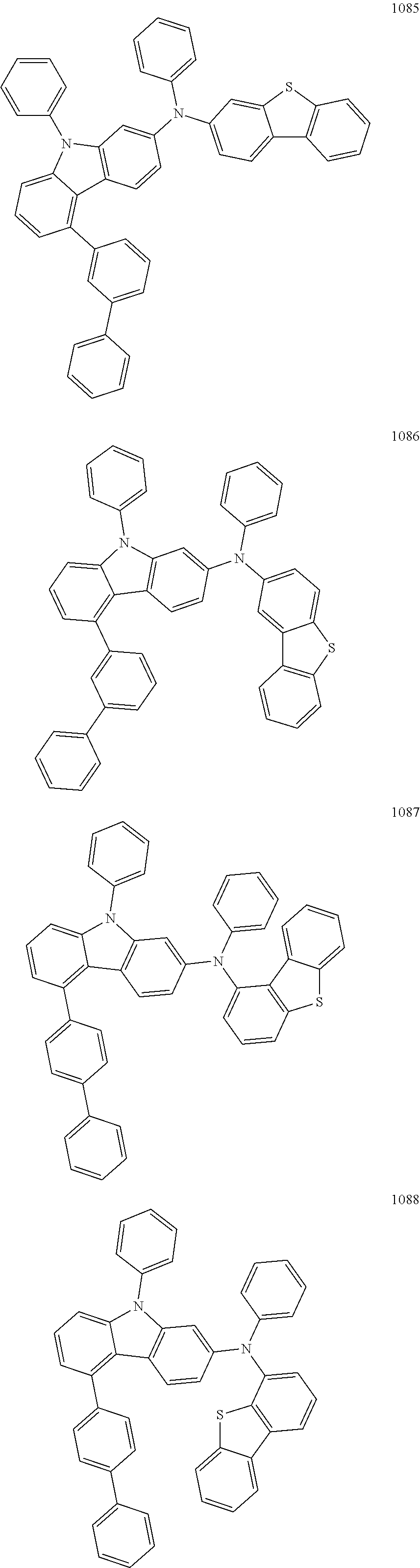

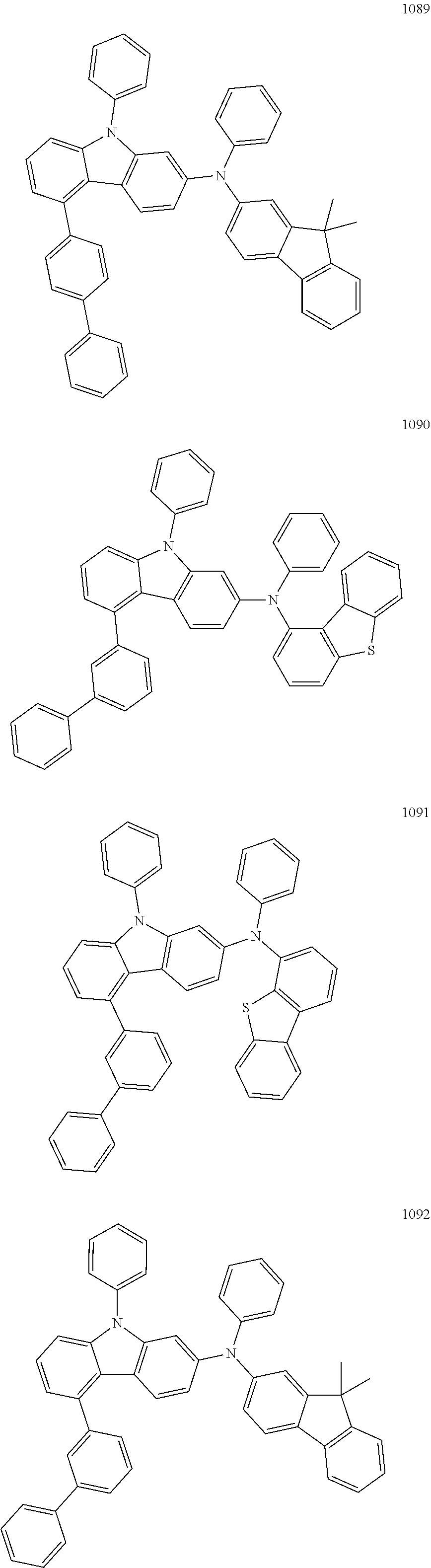 [SMILES: CC1(C)c2ccccc2-c2ccc(N(c3ccccc3)c3ccc4c5c(-c6ccc(-c7ccccc7)cc6)cccc5n(-c5ccccc5)c4c3)cc21 ; c1ccc(-c2cccc(-c3cccc4c3c3ccc(N(c5ccccc5)c5cccc6sc7ccccc7c56)cc3n4-c3ccccc3)c2)cc1 ; c1ccc(-c2cccc(-c3cccc4c3c3ccc(N(c5ccccc5)c5cccc6c5sc5ccccc56)cc3n4-c3ccccc3)c2)cc1 ; CC1(C)c2ccccc2-c2ccc(N(c3ccccc3)c3ccc4c5c(-c6cccc(-c7ccccc7)c6)cccc5n(-c5ccccc5)c4c3)cc21]

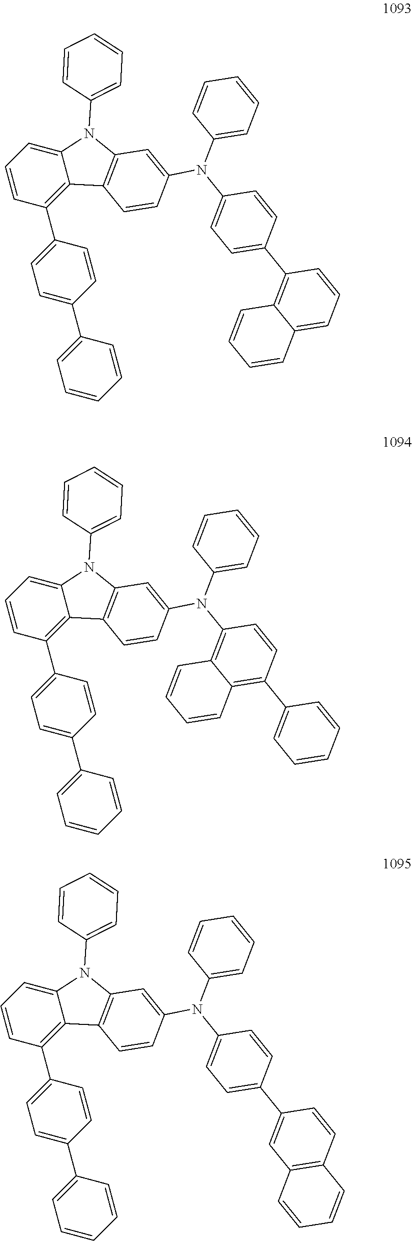

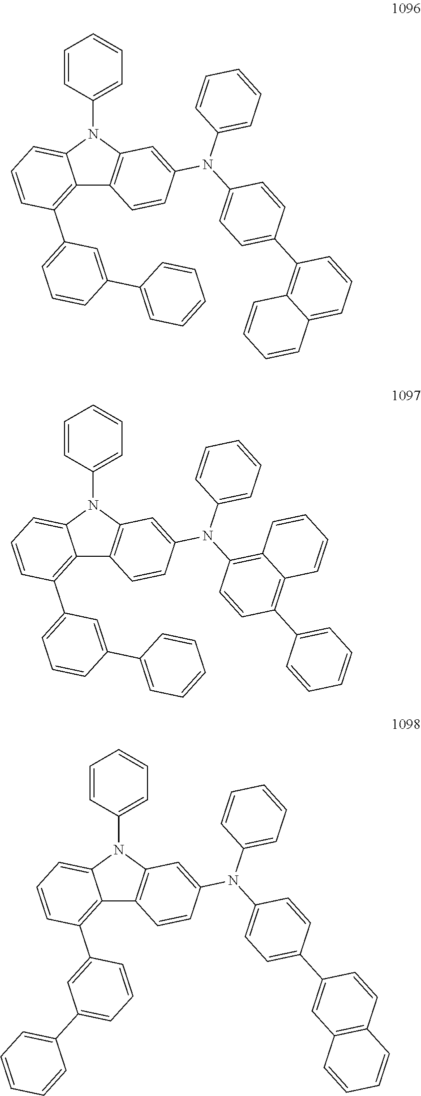 [SMILES: c1ccc(-c2cccc(-c3cccc4c3c3ccc(N(c5ccccc5)c5ccc(-c6cccc7ccccc67)cc5)cc3n4-c3ccccc3)c2)cc1 ; c1ccc(-c2cccc(-c3cccc4c3c3ccc(N(c5ccccc5)c5ccc(-c6ccccc6)c6ccccc56)cc3n4-c3ccccc3)c2)cc1 ; c1ccc(-c2cccc(-c3cccc4c3c3ccc(N(c5ccccc5)c5ccc(-c6ccc7ccccc7c6)cc5)cc3n4-c3ccccc3)c2)cc1]

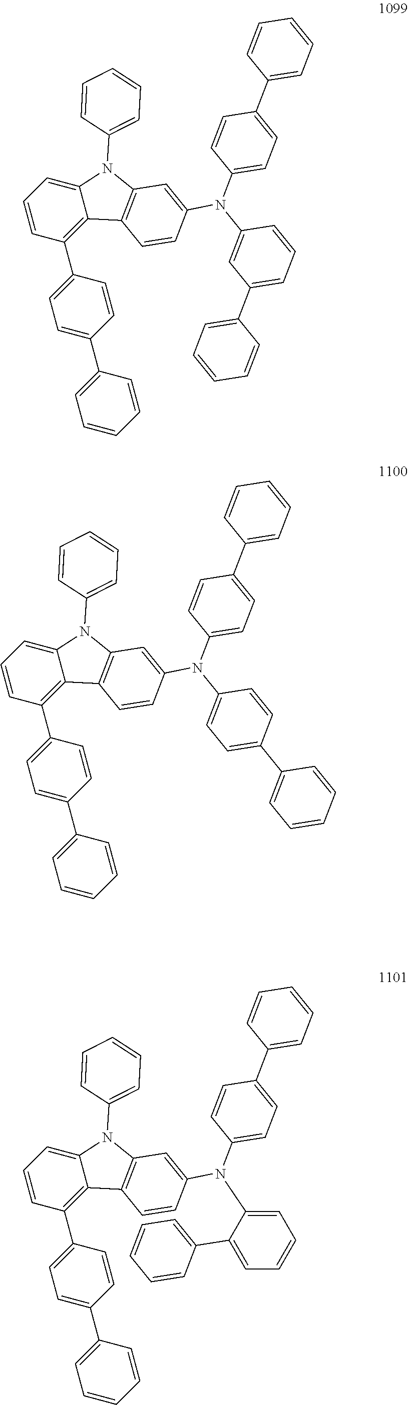 [SMILES: c1ccc(-c2ccc(-c3cccc4c3c3ccc(N(c5ccc(-c6ccccc6)cc5)c5cccc(-c6ccccc6)c5)cc3n4-c3ccccc3)cc2)cc1 ; c1ccc(-c2ccc(-c3cccc4c3c3ccc(N(c5ccc(-c6ccccc6)cc5)c5ccc(-c6ccccc6)cc5)cc3n4-c3ccccc3)cc2)cc1 ; c1ccc(-c2ccc(-c3cccc4c3c3ccc(N(c5ccc(-c6ccccc6)cc5)c5ccccc5-c5ccccc5)cc3n4-c3ccccc3)cc2)cc1]

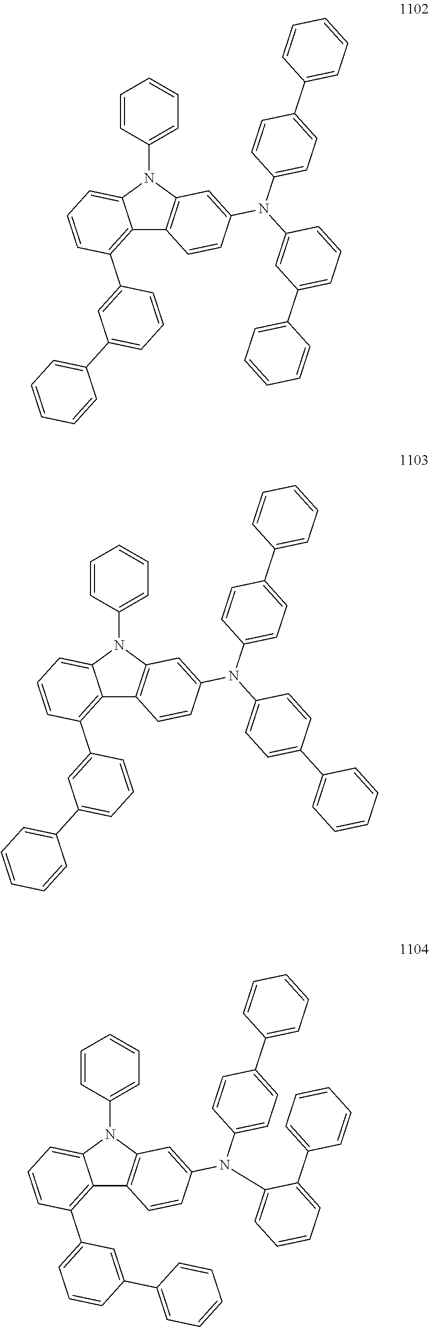 [SMILES: c1ccc(-c2ccc(N(c3cccc(-c4ccccc4)c3)c3ccc4c5c(-c6cccc(-c7ccccc7)c6)cccc5n(-c5ccccc5)c4c3)cc2)cc1 ; c1ccc(-c2ccc(N(c3ccc(-c4ccccc4)cc3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3ccccc3-c3ccccc3)cc2)cc1]

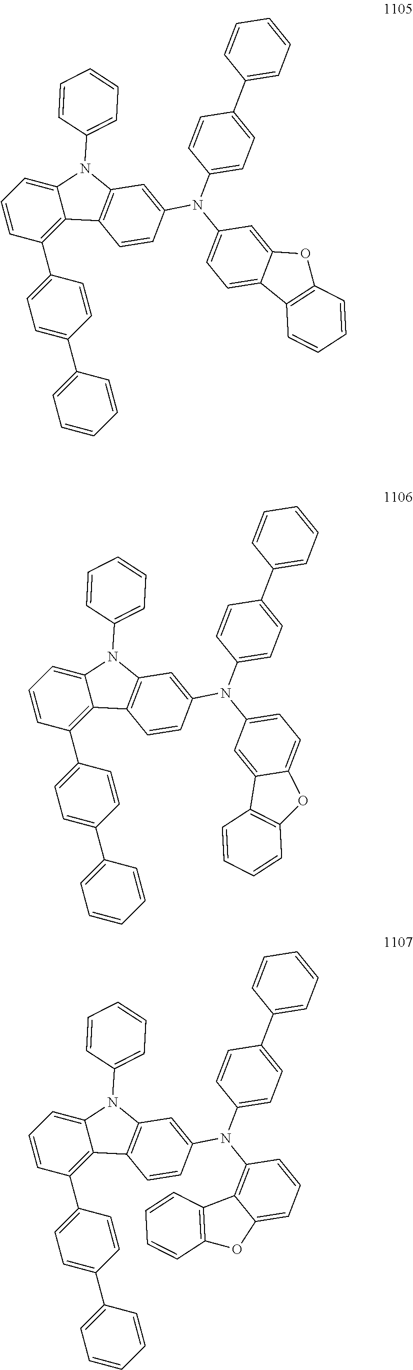 [SMILES: c1ccc(-c2ccc(-c3cccc4c3c3ccc(N(c5ccc(-c6ccccc6)cc5)c5ccc6c(c5)oc5ccccc56)cc3n4-c3ccccc3)cc2)cc1 ; c1ccc(-c2ccc(-c3cccc4c3c3ccc(N(c5ccc(-c6ccccc6)cc5)c5ccc6oc7ccccc7c6c5)cc3n4-c3ccccc3)cc2)cc1 ; c1ccc(-c2ccc(-c3cccc4c3c3ccc(N(c5ccc(-c6ccccc6)cc5)c5cccc6oc7ccccc7c56)cc3n4-c3ccccc3)cc2)cc1]

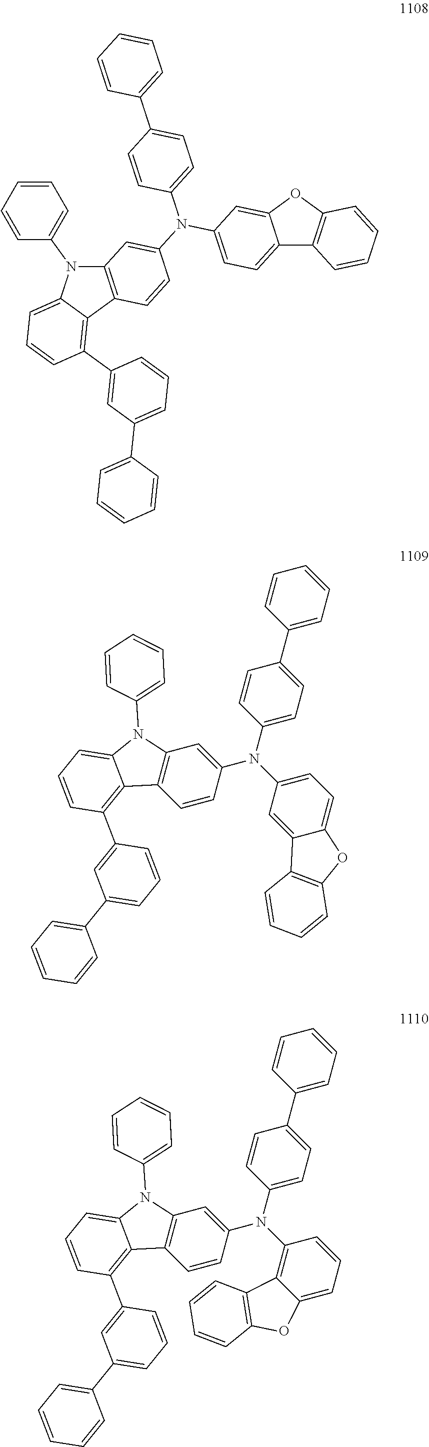 [SMILES: c1ccc(-c2ccc(N(c3ccc4c(c3)oc3ccccc34)c3ccc4c5c(-c6cccc(-c7ccccc7)c6)cccc5n(-c5ccccc5)c4c3)cc2)cc1 ; c1ccc(-c2ccc(N(c3ccc4oc5ccccc5c4c3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3cccc4oc5ccccc5c34)cc2)cc1]

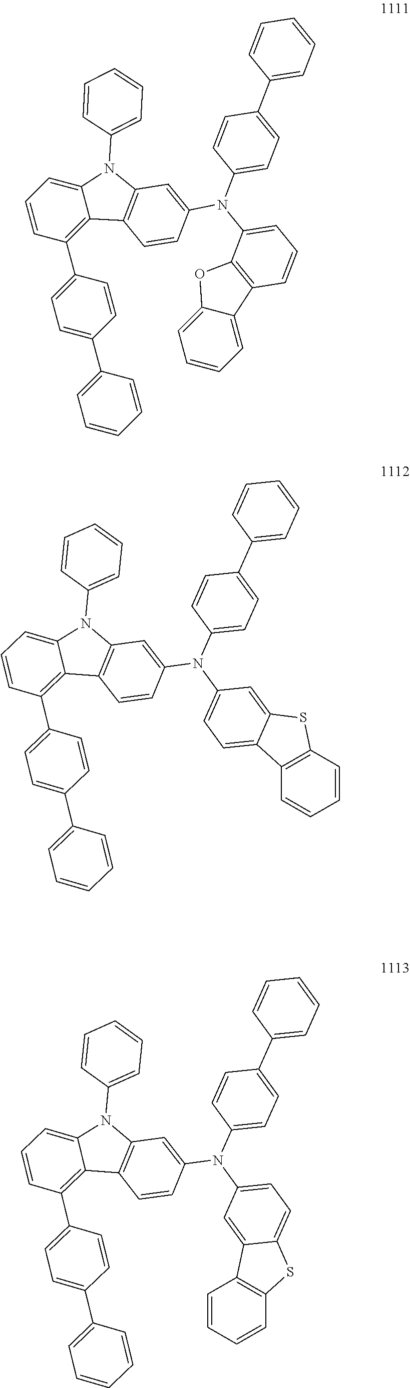 [SMILES: c1ccc(-c2ccc(-c3cccc4c3c3ccc(N(c5ccc(-c6ccccc6)cc5)c5cccc6c5oc5ccccc56)cc3n4-c3ccccc3)cc2)cc1 ; c1ccc(-c2ccc(-c3cccc4c3c3ccc(N(c5ccc(-c6ccccc6)cc5)c5ccc6c(c5)sc5ccccc56)cc3n4-c3ccccc3)cc2)cc1 ; c1ccc(-c2ccc(-c3cccc4c3c3ccc(N(c5ccc(-c6ccccc6)cc5)c5ccc6sc7ccccc7c6c5)cc3n4-c3ccccc3)cc2)cc1]

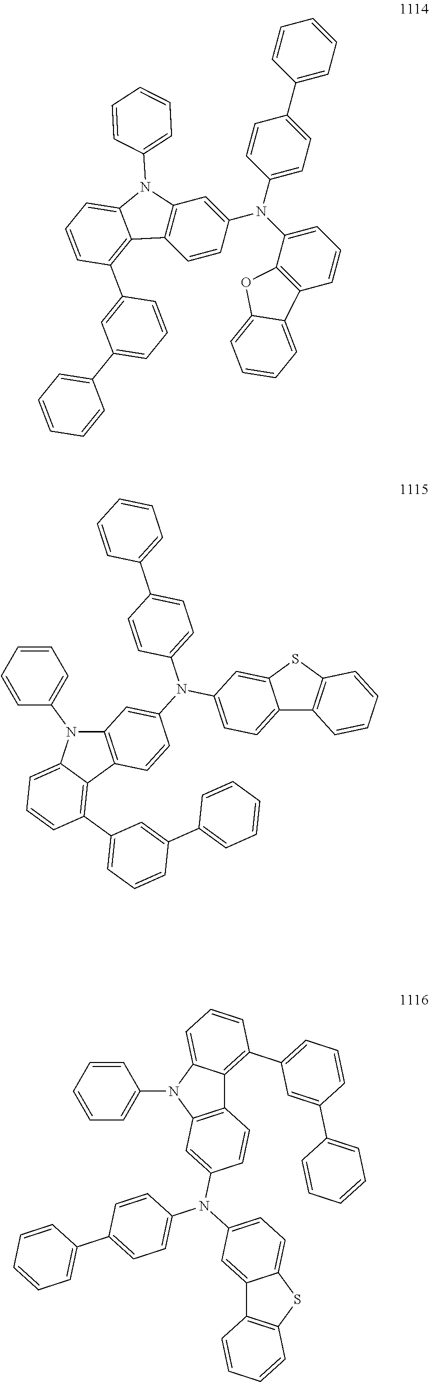 [SMILES: c1ccc(-c2ccc(N(c3ccc4c5c(-c6cccc(-c7ccccc7)c6)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6cccc(-c7ccccc7)c6)cccc5n(-c5ccccc5)c4c3)cc2)cc1 ; c1ccc(-c2ccc(N(c3ccc4sc5ccccc5c4c3)c3ccc4c5c(-c6cccc(-c7ccccc7)c6)cccc5n(-c5ccccc5)c4c3)cc2)cc1]

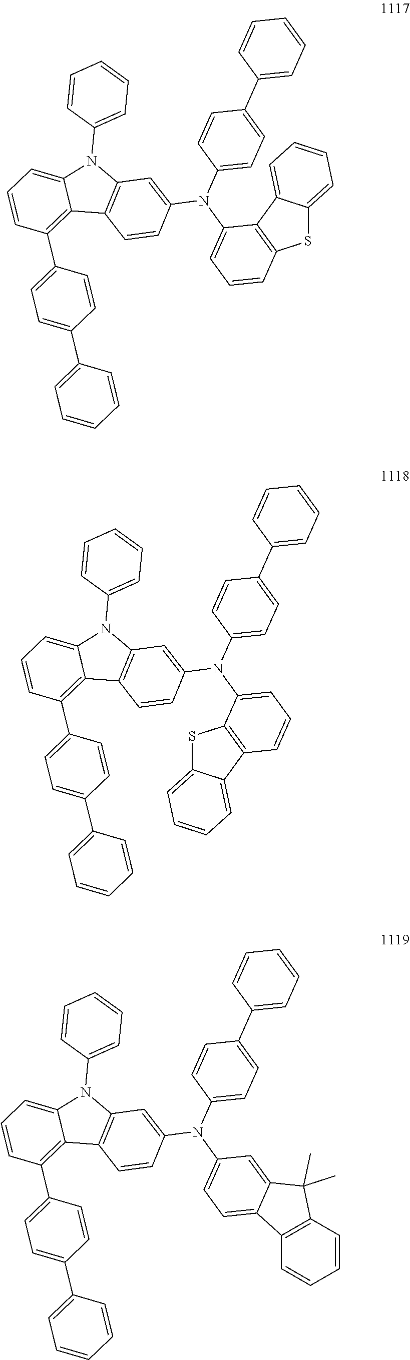 [SMILES: c1ccc(-c2ccc(-c3cccc4c3c3ccc(N(c5ccc(-c6ccccc6)cc5)c5cccc6sc7ccccc7c56)cc3n4-c3ccccc3)cc2)cc1 ; c1ccc(-c2ccc(-c3cccc4c3c3ccc(N(c5ccc(-c6ccccc6)cc5)c5cccc6c5sc5ccccc56)cc3n4-c3ccccc3)cc2)cc1 ; CC1(C)c2ccccc2-c2ccc(N(c3ccc(-c4ccccc4)cc3)c3ccc4c5c(-c6ccc(-c7ccccc7)cc6)cccc5n(-c5ccccc5)c4c3)cc21]

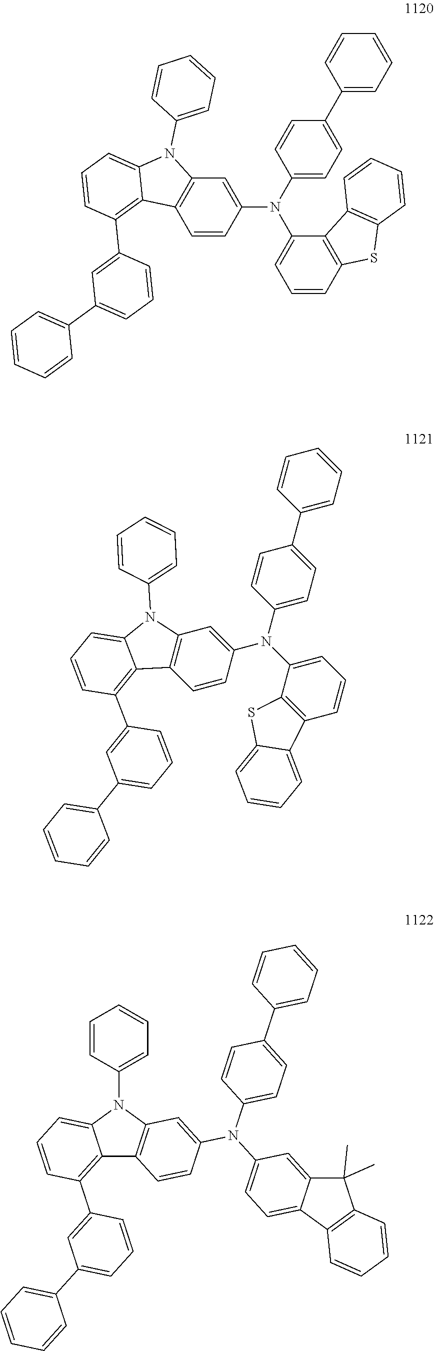 [SMILES: c1ccc(-c2ccc(N(c3ccc4c5c(-c6cccc(-c7ccccc7)c6)cccc5n(-c5ccccc5)c4c3)c3cccc4sc5ccccc5c34)cc2)cc1 ; c1ccc(-c2ccc(N(c3ccc4c5c(-c6cccc(-c7ccccc7)c6)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6cccc(-c7ccccc7)c6)cccc5n(-c5ccccc5)c4c3)cc21]

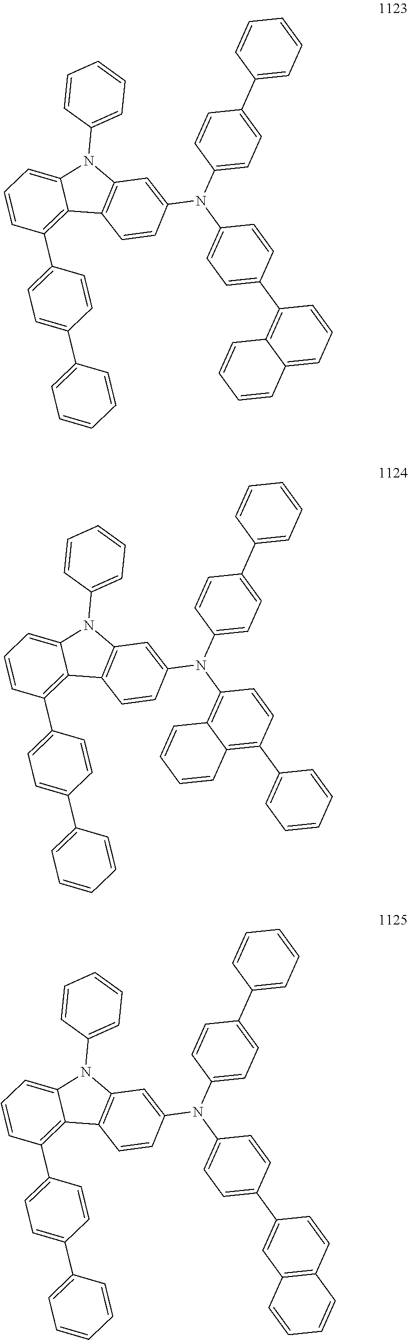 [SMILES: c1ccc(-c2ccc(-c3cccc4c3c3ccc(N(c5ccc(-c6ccccc6)cc5)c5ccc(-c6cccc7ccccc67)cc5)cc3n4-c3ccccc3)cc2)cc1 ; c1ccc(-c2ccc(-c3cccc4c3c3ccc(N(c5ccc(-c6ccccc6)cc5)c5ccc(-c6ccccc6)c6ccccc56)cc3n4-c3ccccc3)cc2)cc1 ; c1ccc(-c2ccc(-c3cccc4c3c3ccc(N(c5ccc(-c6ccccc6)cc5)c5ccc(-c6ccc7ccccc7c6)cc5)cc3n4-c3ccccc3)cc2)cc1]

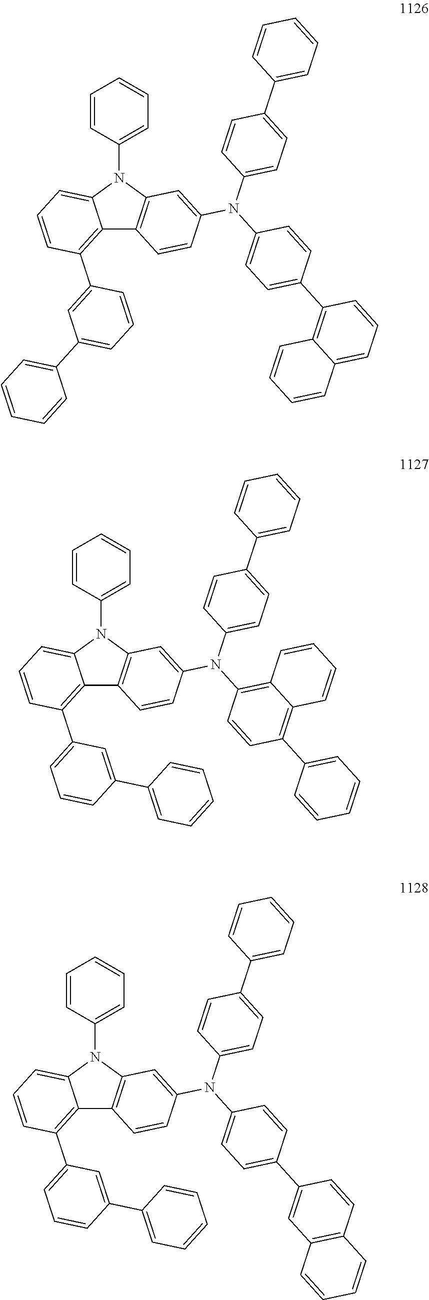 [SMILES: c1ccc(-c2ccc(N(c3ccc(-c4cccc5ccccc45)cc3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3ccc(-c4ccccc4)c4ccccc34)cc2)cc1 ; c1ccc(-c2ccc(N(c3ccc(-c4ccc5ccccc5c4)cc3)c3ccc4c5c(-c6cccc(-c7ccccc7)c6)cccc5n(-c5ccccc5)c4c3)cc2)cc1]

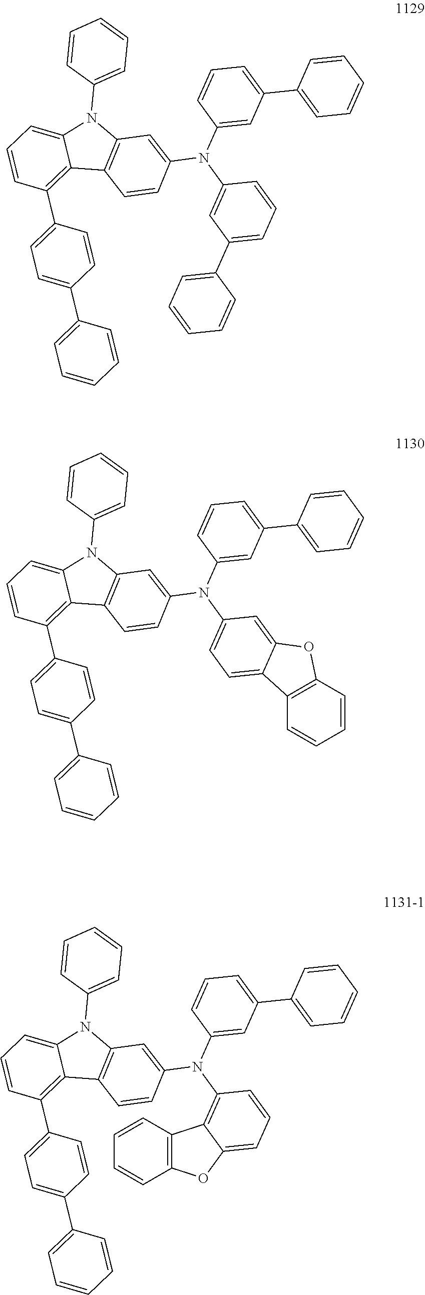 [SMILES: c1ccc(-c2ccc(-c3cccc4c3c3ccc(N(c5cccc(-c6ccccc6)c5)c5cccc(-c6ccccc6)c5)cc3n4-c3ccccc3)cc2)cc1 ; c1ccc(-c2ccc(-c3cccc4c3c3ccc(N(c5cccc(-c6ccccc6)c5)c5ccc6c(c5)oc5ccccc56)cc3n4-c3ccccc3)cc2)cc1 ; c1ccc(-c2ccc(-c3cccc4c3c3ccc(N(c5cccc(-c6ccccc6)c5)c5cccc6oc7ccccc7c56)cc3n4-c3ccccc3)cc2)cc1]

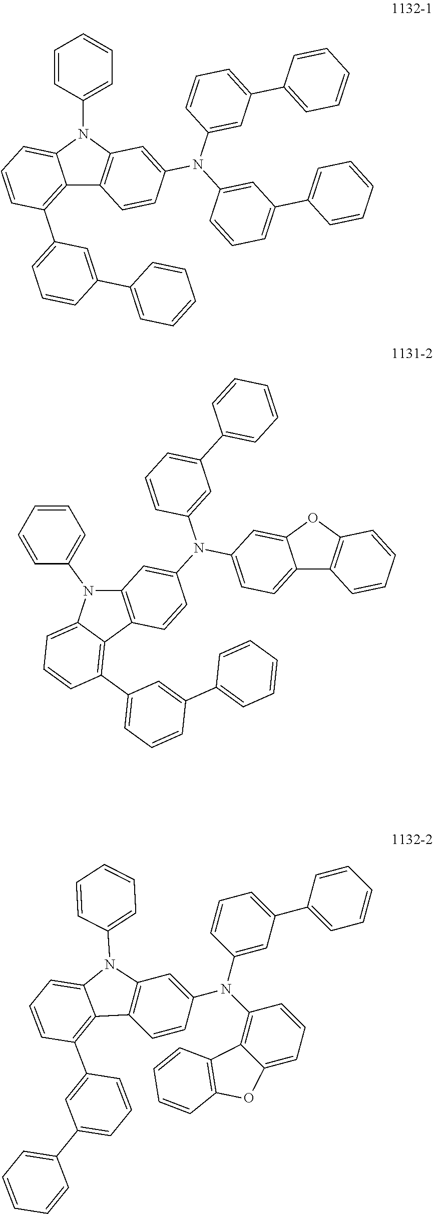 [SMILES: c1ccc(-c2cccc(-c3cccc4c3c3ccc(N(c5cccc(-c6ccccc6)c5)c5cccc(-c6ccccc6)c5)cc3n4-c3ccccc3)c2)cc1 ; c1ccc(-c2cccc(-c3cccc4c3c3ccc(N(c5cccc(-c6ccccc6)c5)c5ccc6c(c5)oc5ccccc56)cc3n4-c3ccccc3)c2)cc1 ; c1ccc(-c2cccc(-c3cccc4c3c3ccc(N(c5cccc(-c6ccccc6)c5)c5cccc6oc7ccccc7c56)cc3n4-c3ccccc3)c2)cc1]

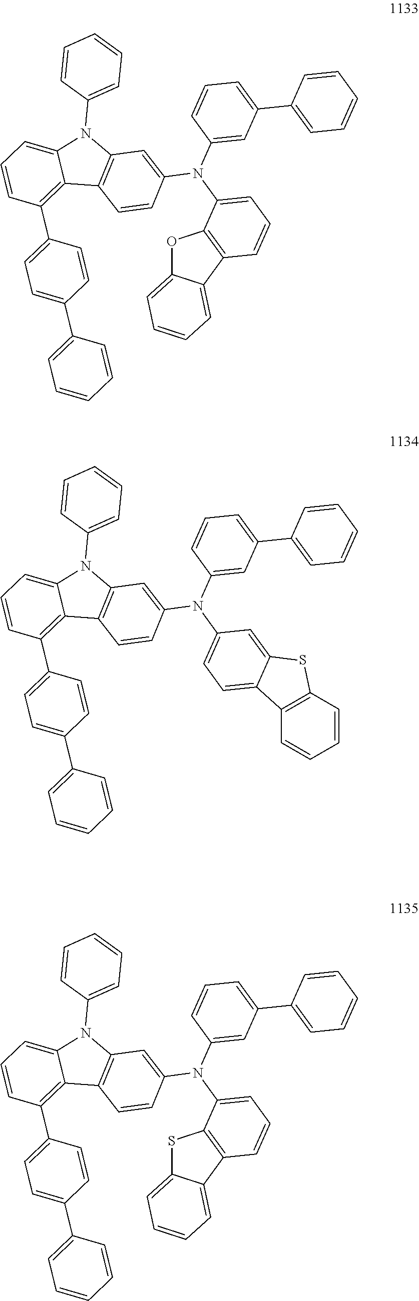 [SMILES: c1ccc(-c2ccc(-c3cccc4c3c3ccc(N(c5cccc(-c6ccccc6)c5)c5cccc6c5oc5ccccc56)cc3n4-c3ccccc3)cc2)cc1 ; c1ccc(-c2ccc(-c3cccc4c3c3ccc(N(c5cccc(-c6ccccc6)c5)c5ccc6c(c5)sc5ccccc56)cc3n4-c3ccccc3)cc2)cc1 ; c1ccc(-c2ccc(-c3cccc4c3c3ccc(N(c5cccc(-c6ccccc6)c5)c5cccc6c5sc5ccccc56)cc3n4-c3ccccc3)cc2)cc1]

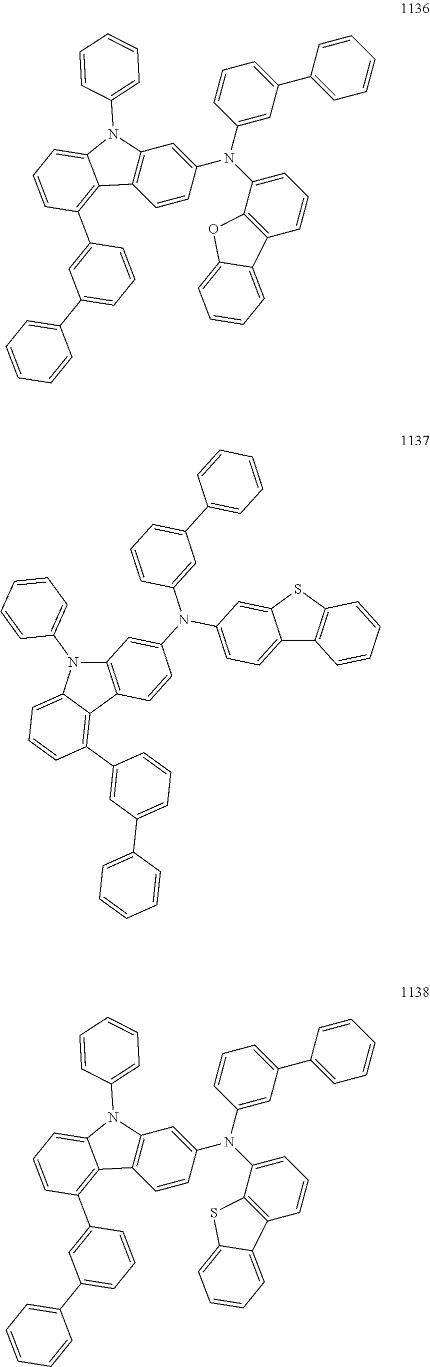 [SMILES: c1ccc(-c2cccc(-c3cccc4c3c3ccc(N(c5cccc(-c6ccccc6)c5)c5cccc6c5oc5ccccc56)cc3n4-c3ccccc3)c2)cc1 ; c1ccc(-c2cccc(-c3cccc4c3c3ccc(N(c5cccc(-c6ccccc6)c5)c5ccc6c(c5)sc5ccccc56)cc3n4-c3ccccc3)c2)cc1 ; c1ccc(-c2cccc(-c3cccc4c3c3ccc(N(c5cccc(-c6ccccc6)c5)c5cccc6c5sc5ccccc56)cc3n4-c3ccccc3)c2)cc1]

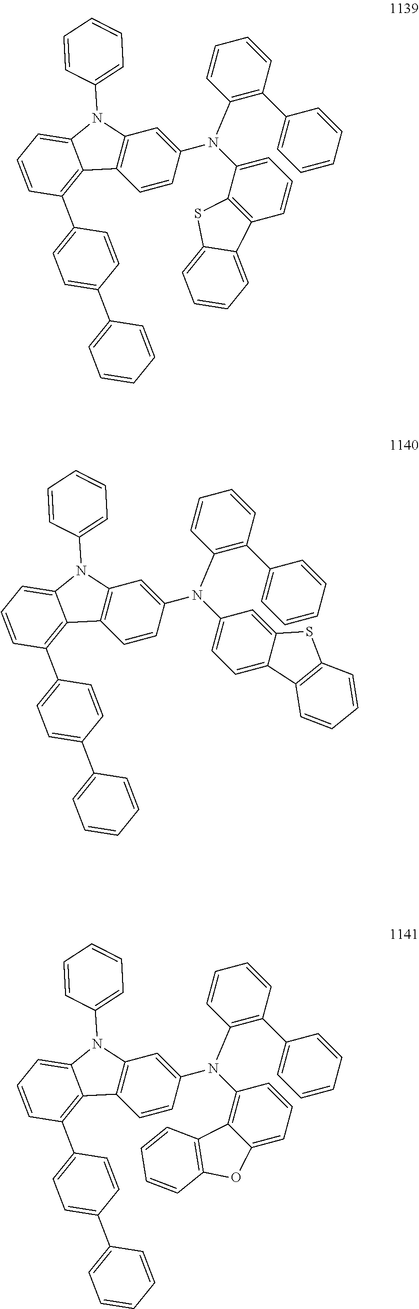 [SMILES: c1ccc(-c2ccc(-c3cccc4c3c3ccc(N(c5ccccc5-c5ccccc5)c5cccc6c5sc5ccccc56)cc3n4-c3ccccc3)cc2)cc1 ; c1ccc(-c2ccc(-c3cccc4c3c3ccc(N(c5ccc6c(c5)sc5ccccc56)c5ccccc5-c5ccccc5)cc3n4-c3ccccc3)cc2)cc1 ; c1ccc(-c2ccc(-c3cccc4c3c3ccc(N(c5ccccc5-c5ccccc5)c5cccc6oc7ccccc7c56)cc3n4-c3ccccc3)cc2)cc1]

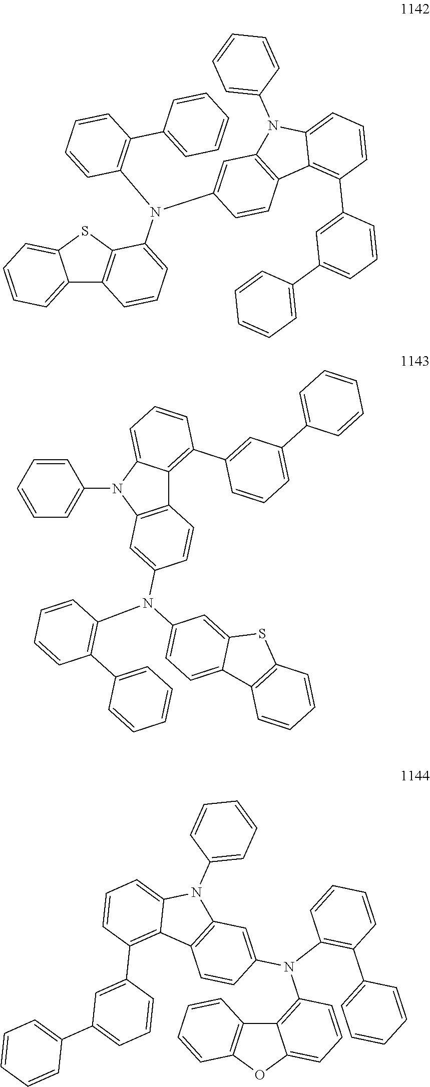 [SMILES: c1ccc(-c2cccc(-c3cccc4c3c3ccc(N(c5ccccc5-c5ccccc5)c5cccc6c5sc5ccccc56)cc3n4-c3ccccc3)c2)cc1 ; c1ccc(-c2cccc(-c3cccc4c3c3ccc(N(c5ccc6c(c5)sc5ccccc56)c5ccccc5-c5ccccc5)cc3n4-c3ccccc3)c2)cc1 ; c1ccc(-c2cccc(-c3cccc4c3c3ccc(N(c5ccccc5-c5ccccc5)c5cccc6oc7ccccc7c56)cc3n4-c3ccccc3)c2)cc1]

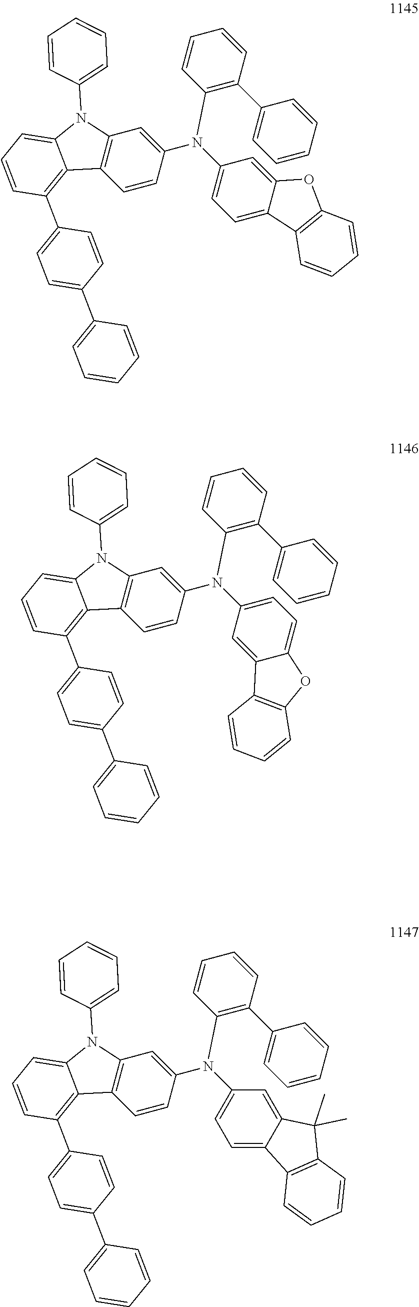 [SMILES: c1ccc(-c2ccc(-c3cccc4c3c3ccc(N(c5ccc6c(c5)oc5ccccc56)c5ccccc5-c5ccccc5)cc3n4-c3ccccc3)cc2)cc1 ; c1ccc(-c2ccc(-c3cccc4c3c3ccc(N(c5ccc6oc7ccccc7c6c5)c5ccccc5-c5ccccc5)cc3n4-c3ccccc3)cc2)cc1 ; CC1(C)c2ccccc2-c2ccc(N(c3ccc4c5c(-c6ccc(-c7ccccc7)cc6)cccc5n(-c5ccccc5)c4c3)c3ccccc3-c3ccccc3)cc21]

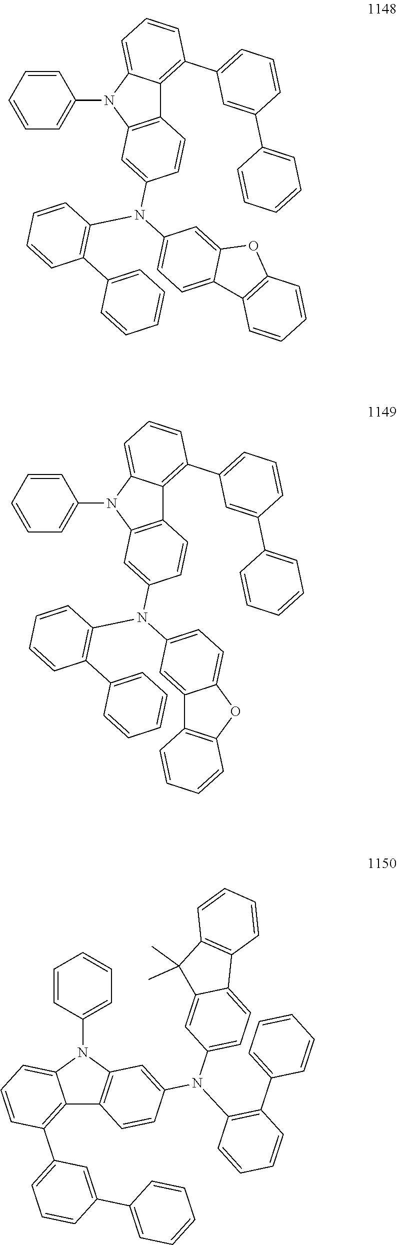 [SMILES: c1ccc(-c2cccc(-c3cccc4c3c3ccc(N(c5ccc6c(c5)oc5ccccc56)c5ccccc5-c5ccccc5)cc3n4-c3ccccc3)c2)cc1 ; c1ccc(-c2cccc(-c3cccc4c3c3ccc(N(c5ccc6oc7ccccc7c6c5)c5ccccc5-c5ccccc5)cc3n4-c3ccccc3)c2)cc1 ; CC1(C)c2ccccc2-c2ccc(N(c3ccc4c5c(-c6cccc(-c7ccccc7)c6)cccc5n(-c5ccccc5)c4c3)c3ccccc3-c3ccccc3)cc21]

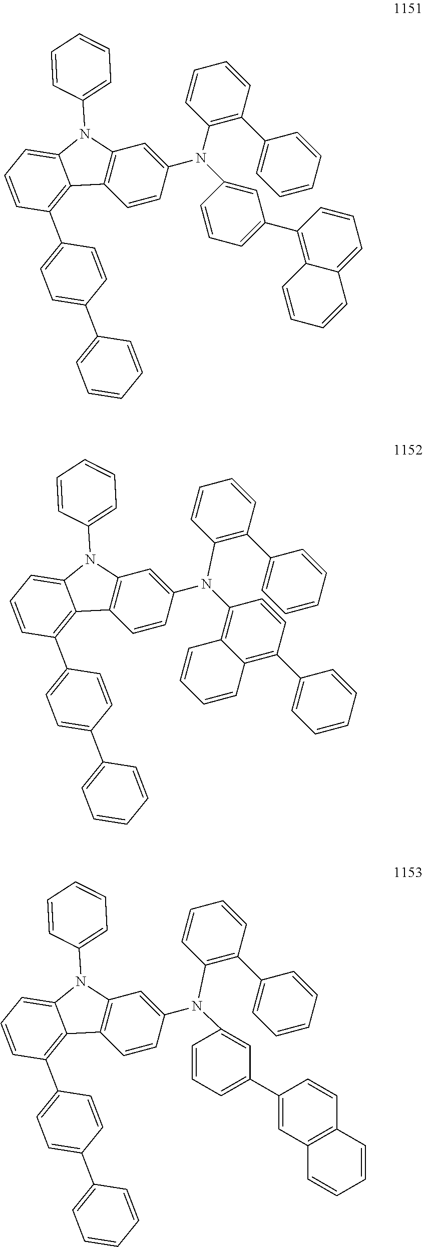 [SMILES: c1ccc(-c2ccc(-c3cccc4c3c3ccc(N(c5cccc(-c6cccc7ccccc67)c5)c5ccccc5-c5ccccc5)cc3n4-c3ccccc3)cc2)cc1 ; c1ccc(-c2ccc(-c3cccc4c3c3ccc(N(c5ccccc5-c5ccccc5)c5ccc(-c6ccccc6)c6ccccc56)cc3n4-c3ccccc3)cc2)cc1 ; c1ccc(-c2ccc(-c3cccc4c3c3ccc(N(c5cccc(-c6ccc7ccccc7c6)c5)c5ccccc5-c5ccccc5)cc3n4-c3ccccc3)cc2)cc1]

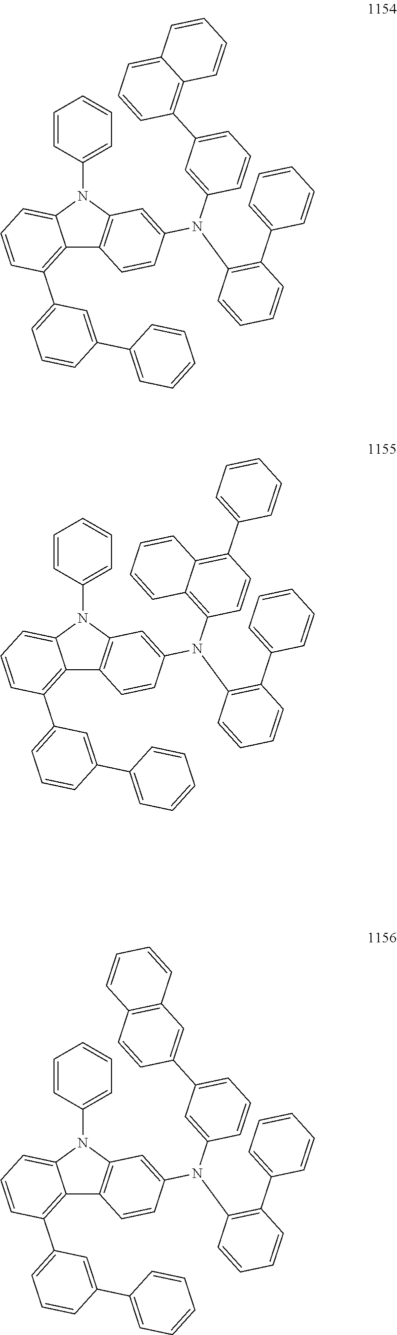 [SMILES: c1ccc(-c2cccc(-c3cccc4c3c3ccc(N(c5cccc(-c6cccc7ccccc67)c5)c5ccccc5-c5ccccc5)cc3n4-c3ccccc3)c2)cc1 ; c1ccc(-c2cccc(-c3cccc4c3c3ccc(N(c5ccccc5-c5ccccc5)c5ccc(-c6ccccc6)c6ccccc56)cc3n4-c3ccccc3)c2)cc1 ; c1ccc(-c2cccc(-c3cccc4c3c3ccc(N(c5cccc(-c6ccc7ccccc7c6)c5)c5ccccc5-c5ccccc5)cc3n4-c3ccccc3)c2)cc1]

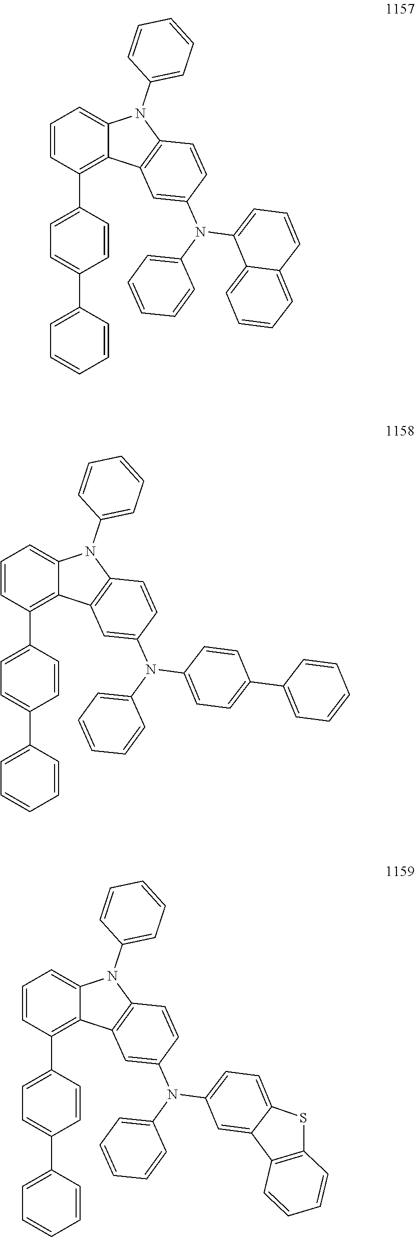 [SMILES: c1ccc(-c2ccc(-c3cccc4c3c3cc(N(c5ccccc5)c5cccc6ccccc56)ccc3n4-c3ccccc3)cc2)cc1 ; c1ccc(-c2ccc(-c3cccc4c3c3cc(N(c5ccccc5)c5ccc(-c6ccccc6)cc5)ccc3n4-c3ccccc3)cc2)cc1 ; c1ccc(-c2ccc(-c3cccc4c3c3cc(N(c5ccccc5)c5ccc6sc7ccccc7c6c5)ccc3n4-c3ccccc3)cc2)cc1]

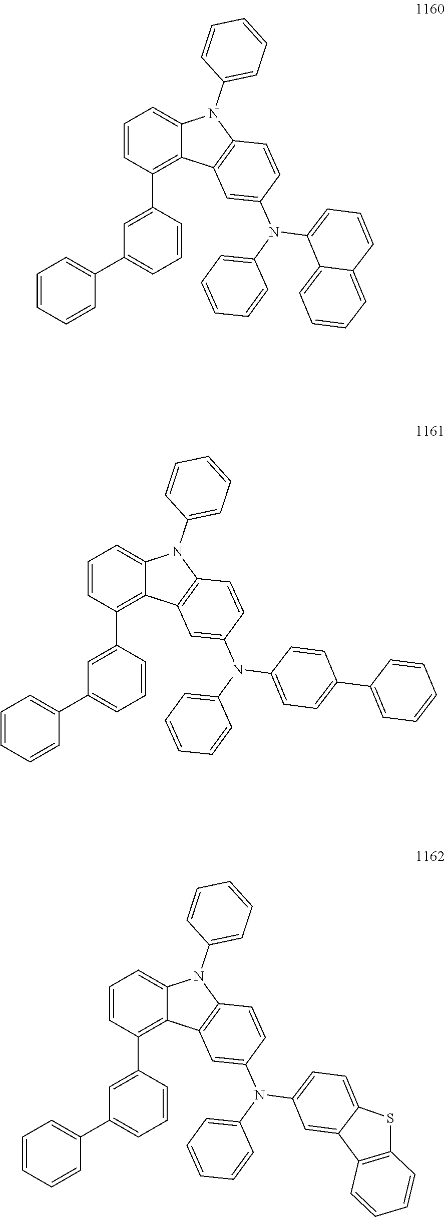 [SMILES: c1ccc(-c2cccc(-c3cccc4c3c3cc(N(c5ccccc5)c5cccc6ccccc56)ccc3n4-c3ccccc3)c2)cc1 ; c1ccc(-c2ccc(N(c3ccccc3)c3ccc4c(c3)c3c(-c5cccc(-c6ccccc6)c5)cccc3n4-c3ccccc3)cc2)cc1 ; c1ccc(-c2cccc(-c3cccc4c3c3cc(N(c5ccccc5)c5ccc6sc7ccccc7c6c5)ccc3n4-c3ccccc3)c2)cc1]

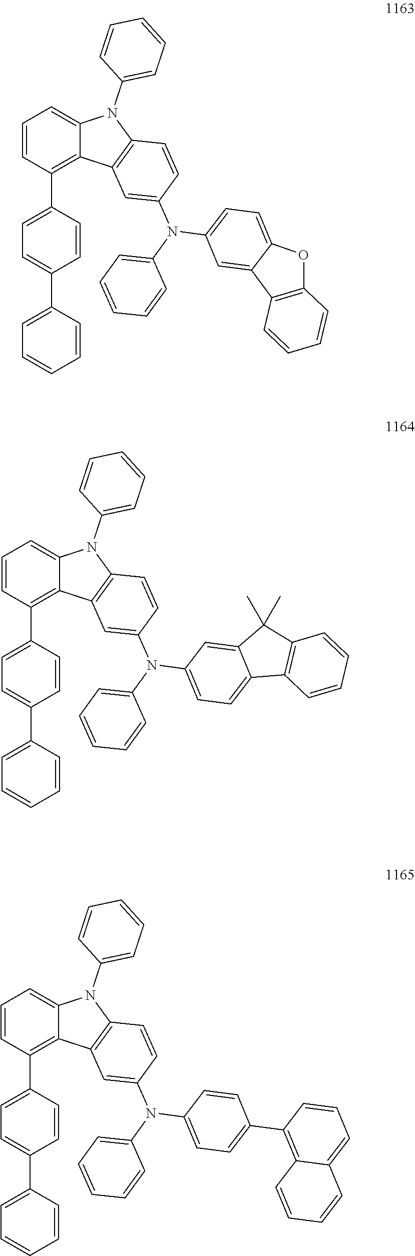 [SMILES: c1ccc(-c2ccc(-c3cccc4c3c3cc(N(c5ccccc5)c5ccc6oc7ccccc7c6c5)ccc3n4-c3ccccc3)cc2)cc1 ; CC1(C)c2ccccc2-c2ccc(N(c3ccccc3)c3ccc4c(c3)c3c(-c5ccc(-c6ccccc6)cc5)cccc3n4-c3ccccc3)cc21 ; c1ccc(-c2ccc(-c3cccc4c3c3cc(N(c5ccccc5)c5ccc(-c6cccc7ccccc67)cc5)ccc3n4-c3ccccc3)cc2)cc1]

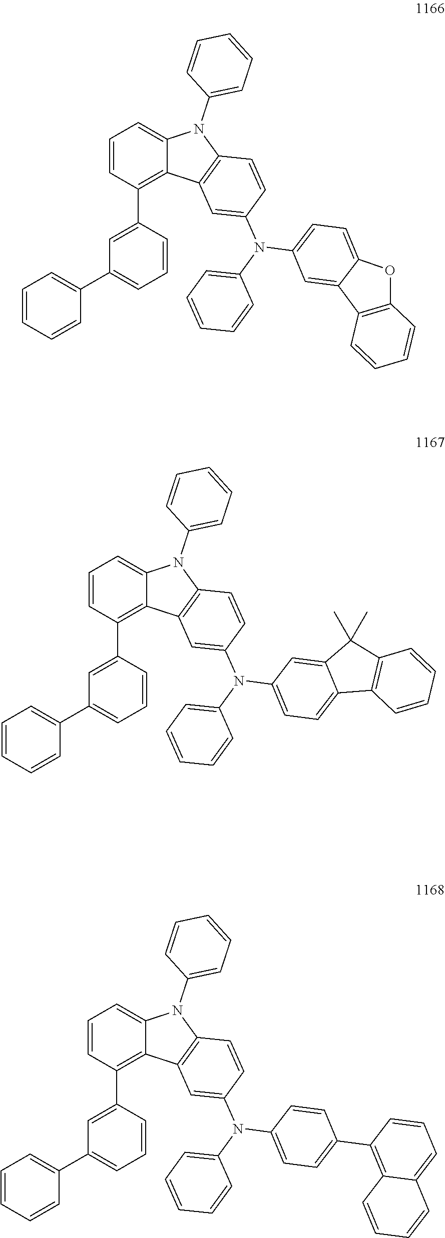 [SMILES: c1ccc(-c2cccc(-c3cccc4c3c3cc(N(c5ccccc5)c5ccc6oc7ccccc7c6c5)ccc3n4-c3ccccc3)c2)cc1 ; CC1(C)c2ccccc2-c2ccc(N(c3ccccc3)c3ccc4c(c3)c3c(-c5cccc(-c6ccccc6)c5)cccc3n4-c3ccccc3)cc21 ; c1ccc(-c2cccc(-c3cccc4c3c3cc(N(c5ccccc5)c5ccc(-c6cccc7ccccc67)cc5)ccc3n4-c3ccccc3)c2)cc1]

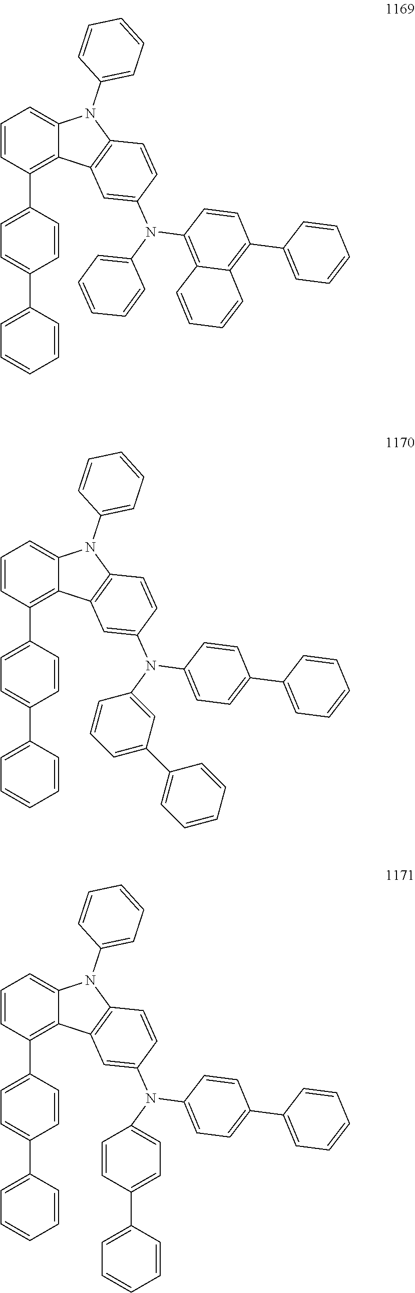 [SMILES: c1ccc(-c2ccc(-c3cccc4c3c3cc(N(c5ccccc5)c5ccc(-c6ccccc6)c6ccccc56)ccc3n4-c3ccccc3)cc2)cc1 ; c1ccc(-c2ccc(-c3cccc4c3c3cc(N(c5ccc(-c6ccccc6)cc5)c5cccc(-c6ccccc6)c5)ccc3n4-c3ccccc3)cc2)cc1 ; c1ccc(-c2ccc(-c3cccc4c3c3cc(N(c5ccc(-c6ccccc6)cc5)c5ccc(-c6ccccc6)cc5)ccc3n4-c3ccccc3)cc2)cc1]

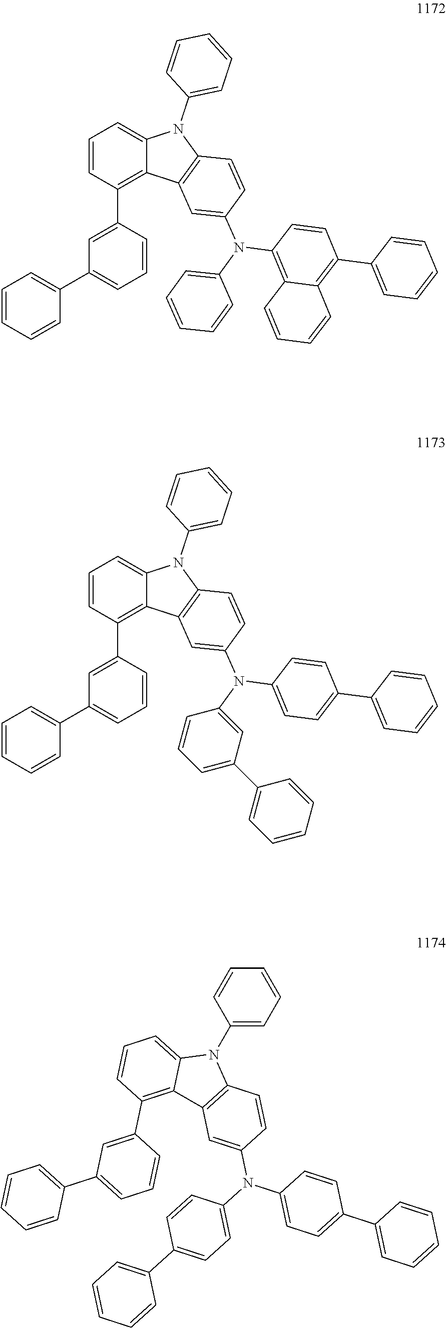 [SMILES: c1ccc(-c2cccc(-c3cccc4c3c3cc(N(c5ccccc5)c5ccc(-c6ccccc6)c6ccccc56)ccc3n4-c3ccccc3)c2)cc1 ; c1ccc(-c2ccc(N(c3cccc(-c4ccccc4)c3)c3ccc4c(c3)c3c(-c5cccc(-c6ccccc6)c5)cccc3n4-c3ccccc3)cc2)cc1 ; c1ccc(-c2ccc(N(c3ccc(-c4ccccc4)cc3)c3ccc4c(c3)c3c(-c5cccc(-c6ccccc6)c5)cccc3n4-c3ccccc3)cc2)cc1]

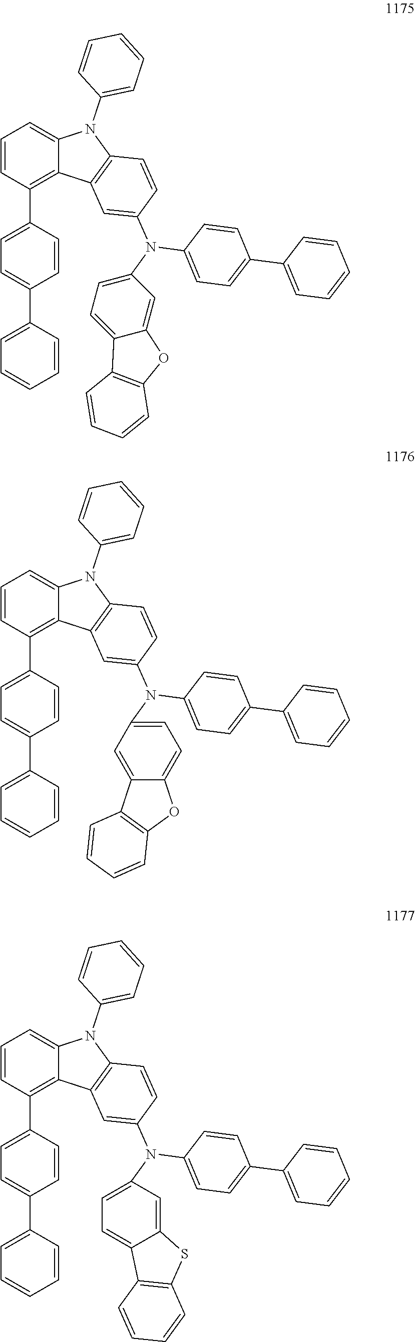

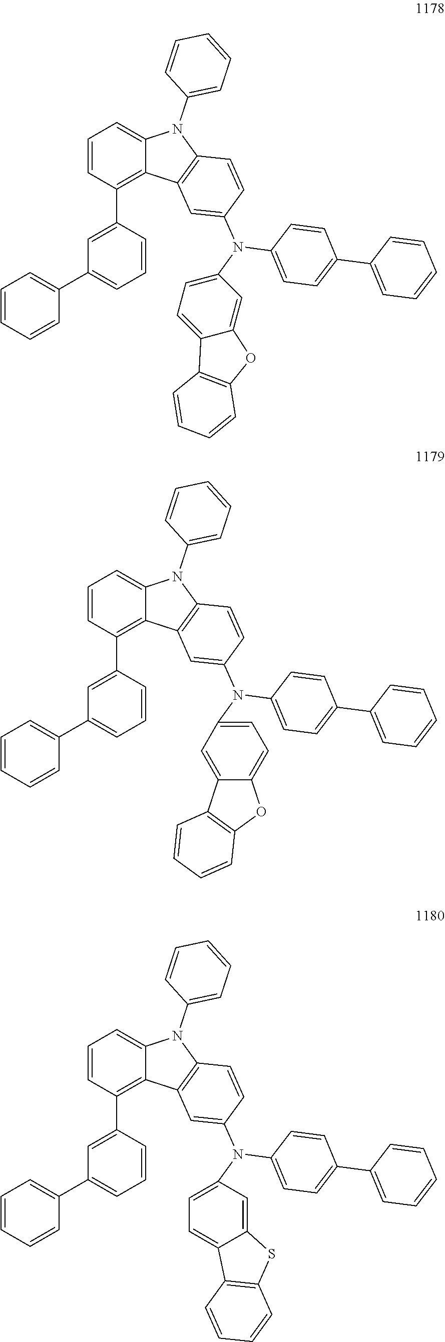

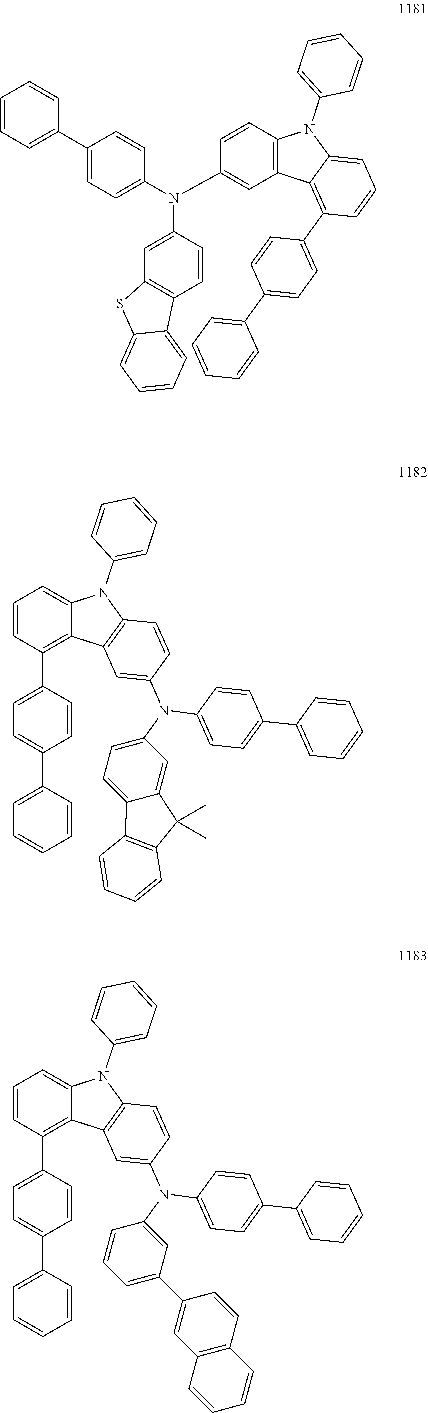 [SMILES: c1ccc(-c2ccc(-c3cccc4c3c3cc(N(c5ccc(-c6ccccc6)cc5)c5ccc6c(c5)sc5ccccc56)ccc3n4-c3ccccc3)cc2)cc1 ; CC1(C)c2ccccc2-c2ccc(N(c3ccc(-c4ccccc4)cc3)c3ccc4c(c3)c3c(-c5ccc(-c6ccccc6)cc5)cccc3n4-c3ccccc3)cc21 ; c1ccc(-c2ccc(-c3cccc4c3c3cc(N(c5ccc(-c6ccccc6)cc5)c5cccc(-c6ccc7ccccc7c6)c5)ccc3n4-c3ccccc3)cc2)cc1]

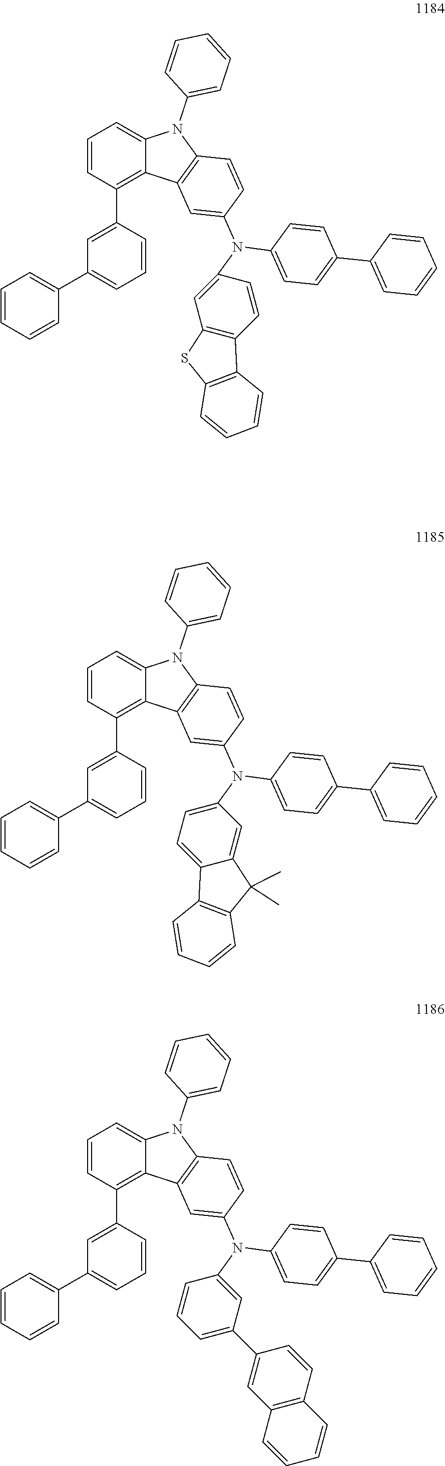 [SMILES: c1ccc(-c2ccc(N(c3ccc4c(c3)sc3ccccc34)c3ccc4c(c3)c3c(-c5cccc(-c6ccccc6)c5)cccc3n4-c3ccccc3)cc2)cc1 ; CC1(C)c2ccccc2-c2ccc(N(c3ccc(-c4ccccc4)cc3)c3ccc4c(c3)c3c(-c5cccc(-c6ccccc6)c5)cccc3n4-c3ccccc3)cc21 ; c1ccc(-c2ccc(N(c3cccc(-c4ccc5ccccc5c4)c3)c3ccc4c(c3)c3c(-c5cccc(-c6ccccc6)c5)cccc3n4-c3ccccc3)cc2)cc1]

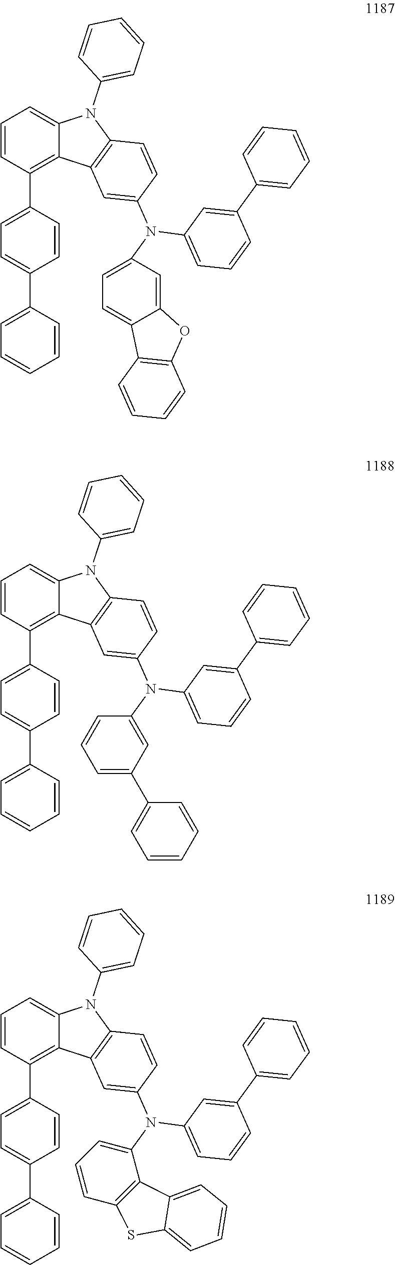 [SMILES: c1ccc(-c2ccc(-c3cccc4c3c3cc(N(c5cccc(-c6ccccc6)c5)c5ccc6c(c5)oc5ccccc56)ccc3n4-c3ccccc3)cc2)cc1 ; c1ccc(-c2ccc(-c3cccc4c3c3cc(N(c5cccc(-c6ccccc6)c5)c5cccc(-c6ccccc6)c5)ccc3n4-c3ccccc3)cc2)cc1 ; c1ccc(-c2ccc(-c3cccc4c3c3cc(N(c5cccc(-c6ccccc6)c5)c5cccc6sc7ccccc7c56)ccc3n4-c3ccccc3)cc2)cc1]

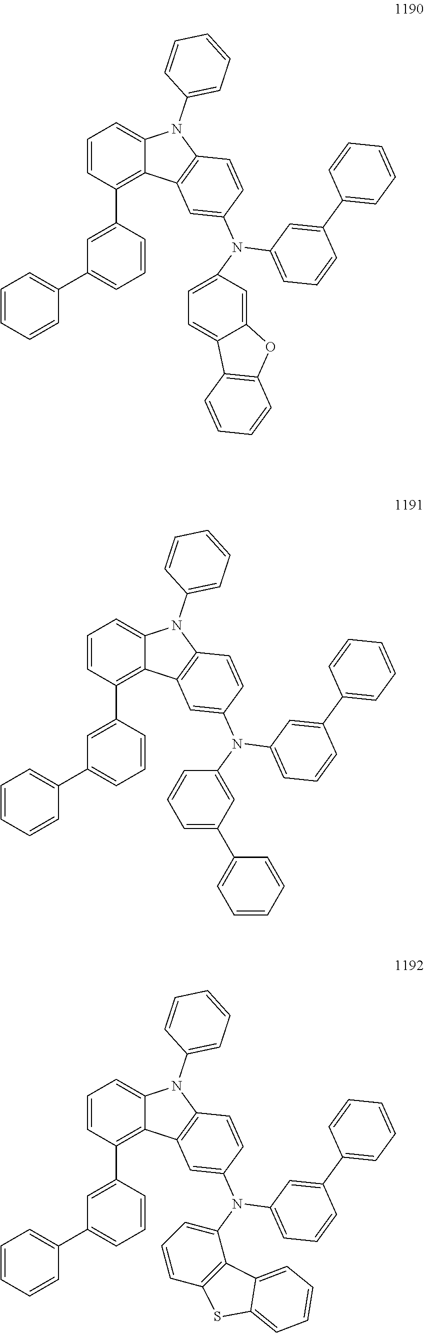 [SMILES: c1ccc(-c2cccc(-c3cccc4c3c3cc(N(c5cccc(-c6ccccc6)c5)c5ccc6c(c5)oc5ccccc56)ccc3n4-c3ccccc3)c2)cc1 ; c1ccc(-c2cccc(-c3cccc4c3c3cc(N(c5cccc(-c6ccccc6)c5)c5cccc(-c6ccccc6)c5)ccc3n4-c3ccccc3)c2)cc1 ; c1ccc(-c2cccc(-c3cccc4c3c3cc(N(c5cccc(-c6ccccc6)c5)c5cccc6sc7ccccc7c56)ccc3n4-c3ccccc3)c2)cc1]

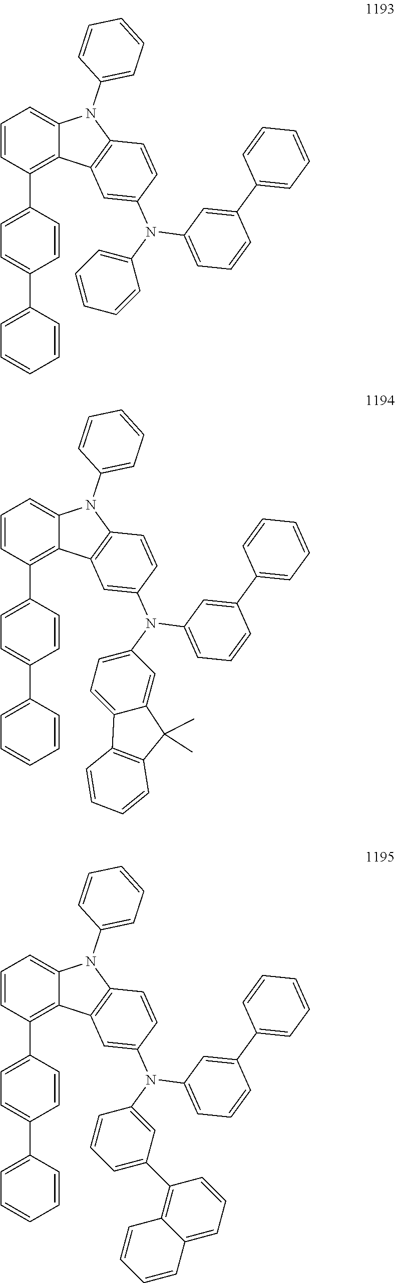 [SMILES: c1ccc(-c2ccc(-c3cccc4c3c3cc(N(c5ccccc5)c5cccc(-c6ccccc6)c5)ccc3n4-c3ccccc3)cc2)cc1 ; CC1(C)c2ccccc2-c2ccc(N(c3cccc(-c4ccccc4)c3)c3ccc4c(c3)c3c(-c5ccc(-c6ccccc6)cc5)cccc3n4-c3ccccc3)cc21 ; c1ccc(-c2ccc(-c3cccc4c3c3cc(N(c5cccc(-c6ccccc6)c5)c5cccc(-c6cccc7ccccc67)c5)ccc3n4-c3ccccc3)cc2)cc1]

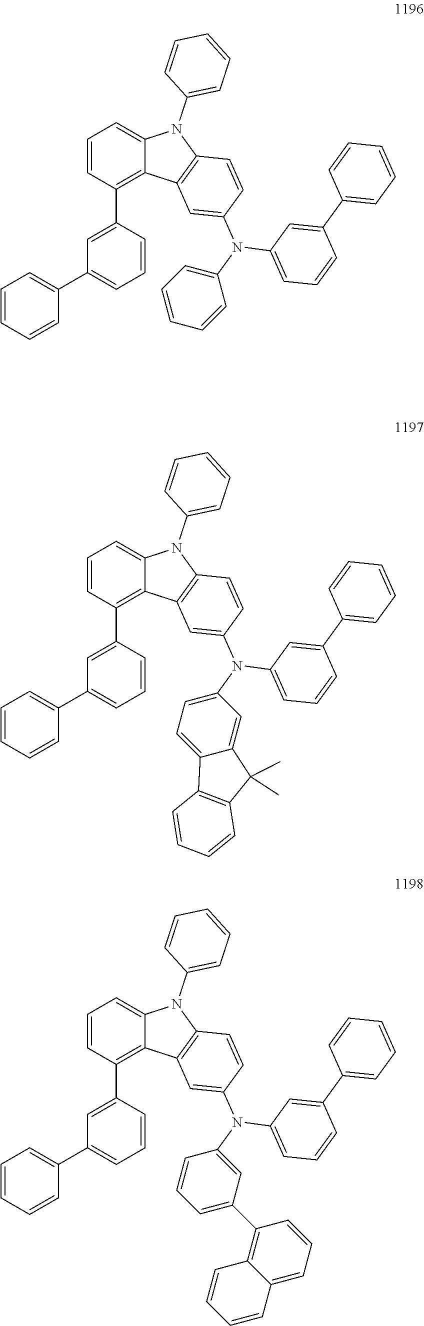 [SMILES: c1ccc(-c2cccc(-c3cccc4c3c3cc(N(c5ccccc5)c5cccc(-c6ccccc6)c5)ccc3n4-c3ccccc3)c2)cc1 ; CC1(C)c2ccccc2-c2ccc(N(c3cccc(-c4ccccc4)c3)c3ccc4c(c3)c3c(-c5cccc(-c6ccccc6)c5)cccc3n4-c3ccccc3)cc21 ; c1ccc(-c2cccc(-c3cccc4c3c3cc(N(c5cccc(-c6ccccc6)c5)c5cccc(-c6cccc7ccccc67)c5)ccc3n4-c3ccccc3)c2)cc1]

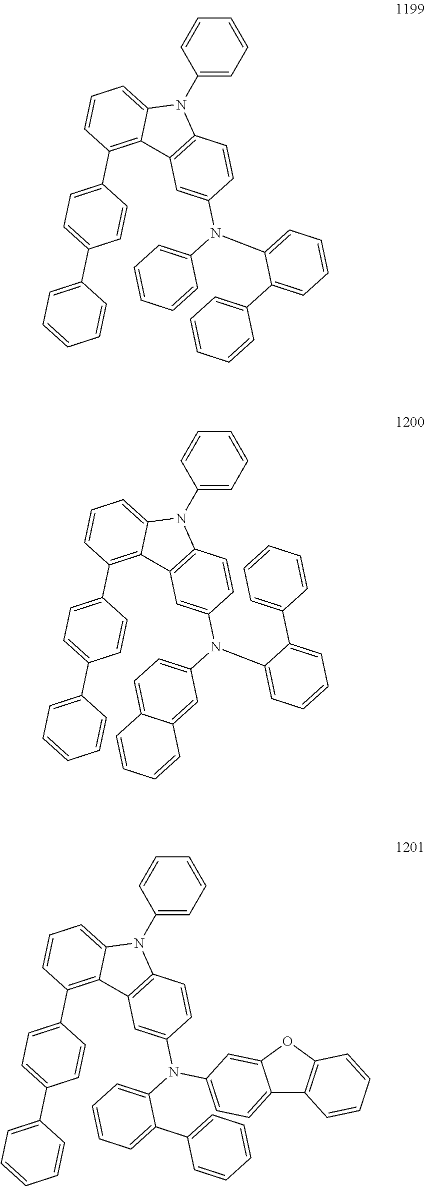 [SMILES: c1ccc(-c2ccc(-c3cccc4c3c3cc(N(c5ccccc5)c5ccccc5-c5ccccc5)ccc3n4-c3ccccc3)cc2)cc1 ; c1ccc(-c2ccc(-c3cccc4c3c3cc(N(c5ccc6ccccc6c5)c5ccccc5-c5ccccc5)ccc3n4-c3ccccc3)cc2)cc1 ; c1ccc(-c2ccc(-c3cccc4c3c3cc(N(c5ccc6c(c5)oc5ccccc56)c5ccccc5-c5ccccc5)ccc3n4-c3ccccc3)cc2)cc1]

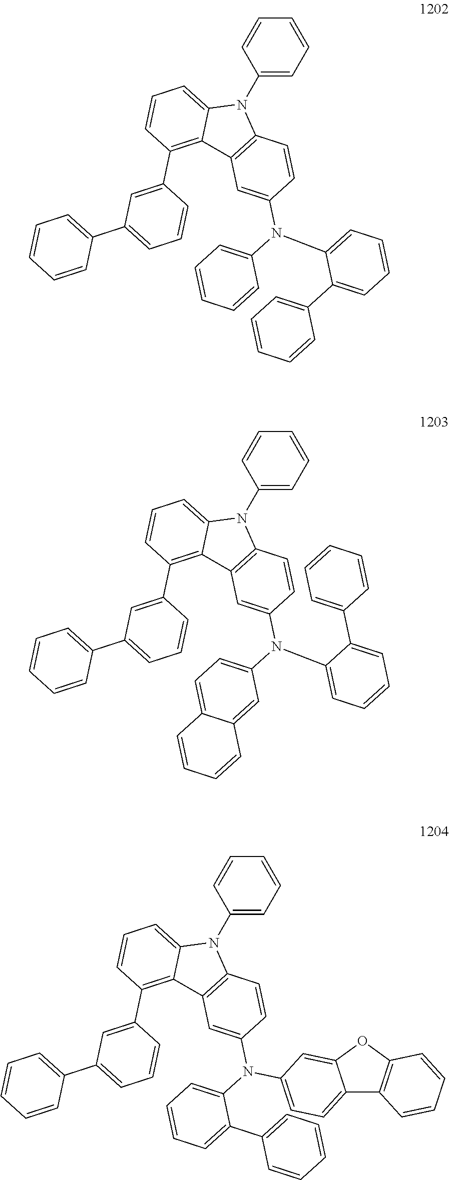 [SMILES: c1ccc(-c2cccc(-c3cccc4c3c3cc(N(c5ccccc5)c5ccccc5-c5ccccc5)ccc3n4-c3ccccc3)c2)cc1 ; c1ccc(-c2cccc(-c3cccc4c3c3cc(N(c5ccc6ccccc6c5)c5ccccc5-c5ccccc5)ccc3n4-c3ccccc3)c2)cc1 ; c1ccc(-c2cccc(-c3cccc4c3c3cc(N(c5ccc6c(c5)oc5ccccc56)c5ccccc5-c5ccccc5)ccc3n4-c3ccccc3)c2)cc1]

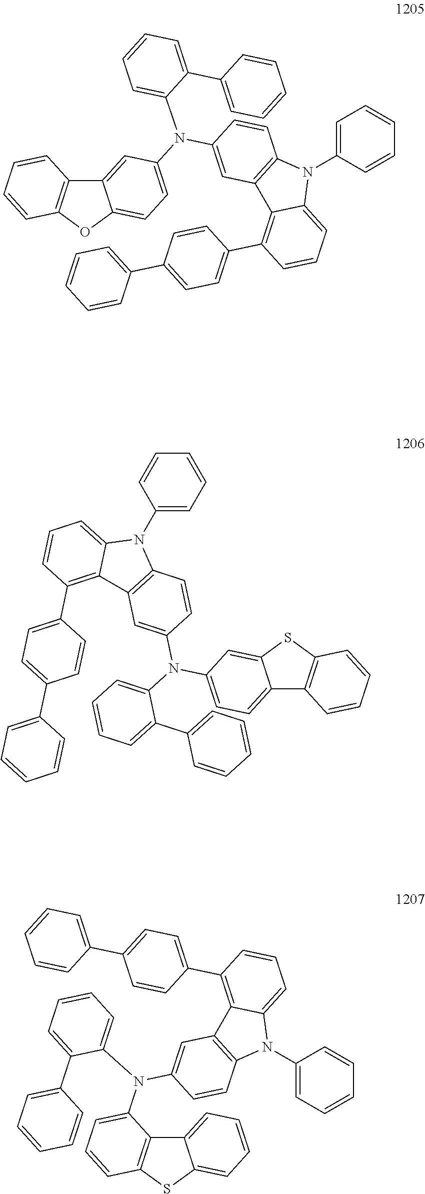 [SMILES: c1ccc(-c2ccc(-c3cccc4c3c3cc(N(c5ccc6oc7ccccc7c6c5)c5ccccc5-c5ccccc5)ccc3n4-c3ccccc3)cc2)cc1 ; c1ccc(-c2ccc(-c3cccc4c3c3cc(N(c5ccc6c(c5)sc5ccccc56)c5ccccc5-c5ccccc5)ccc3n4-c3ccccc3)cc2)cc1 ; c1ccc(-c2ccc(-c3cccc4c3c3cc(N(c5ccccc5-c5ccccc5)c5cccc6sc7ccccc7c56)ccc3n4-c3ccccc3)cc2)cc1]

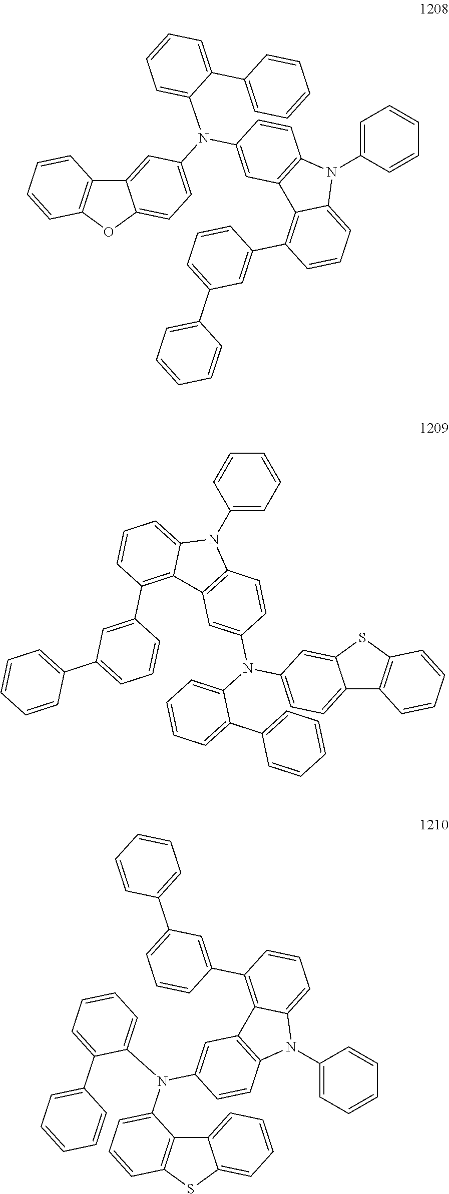 [SMILES: c1ccc(-c2cccc(-c3cccc4c3c3cc(N(c5ccc6oc7ccccc7c6c5)c5ccccc5-c5ccccc5)ccc3n4-c3ccccc3)c2)cc1 ; c1ccc(-c2cccc(-c3cccc4c3c3cc(N(c5ccc6c(c5)sc5ccccc56)c5ccccc5-c5ccccc5)ccc3n4-c3ccccc3)c2)cc1 ; c1ccc(-c2cccc(-c3cccc4c3c3cc(N(c5ccccc5-c5ccccc5)c5cccc6sc7ccccc7c56)ccc3n4-c3ccccc3)c2)cc1]

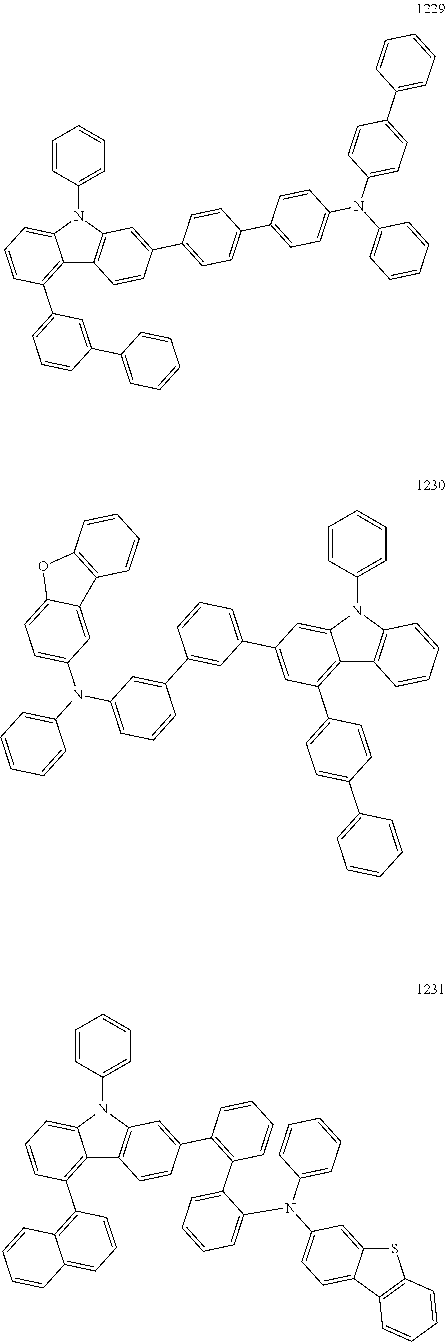 [SMILES: c1ccc(-c2ccc(N(c3ccccc3)c3ccc(-c4ccc(-c5ccc6c7c(-c8cccc(-c9ccccc9)c8)cccc7n(-c7ccccc7)c6c5)cc4)cc3)cc2)cc1 ; c1ccc(-c2ccc(-c3cc(-c4cccc(-c5cccc(N(c6ccccc6)c6ccc7oc8ccccc8c7c6)c5)c4)cc4c3c3ccccc3n4-c3ccccc3)cc2)cc1 ; c1ccc(N(c2ccc3c(c2)sc2ccccc23)c2ccccc2-c2ccccc2-c2ccc3c4c(-c5cccc6ccccc56)cccc4n(-c4ccccc4)c3c2)cc1]

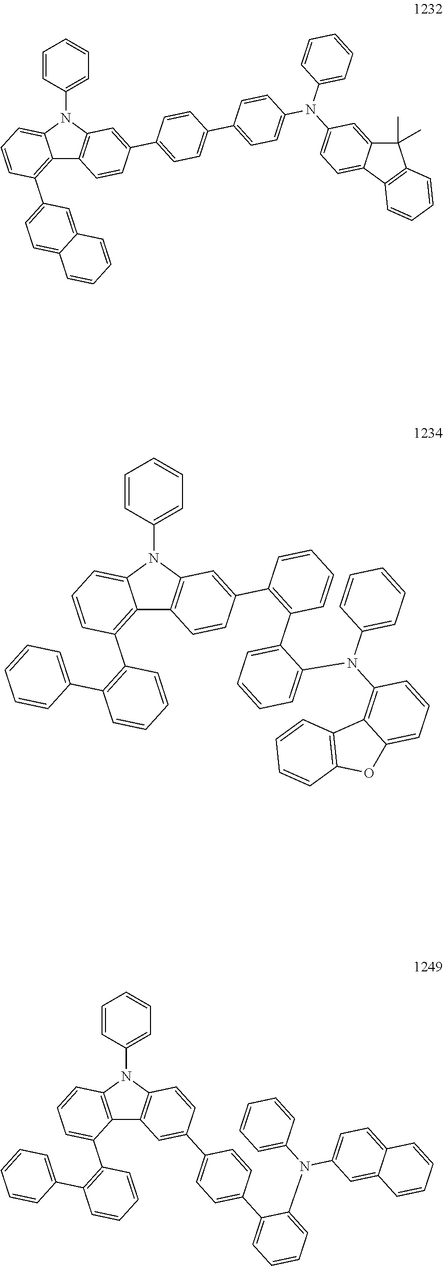 [SMILES: CC1(C)c2ccccc2-c2ccc(N(c3ccccc3)c3ccc(-c4ccc(-c5ccc6c7c(-c8ccc9ccccc9c8)cccc7n(-c7ccccc7)c6c5)cc4)cc3)cc21 ; c1ccc(-c2ccccc2-c2cccc3c2c2ccc(-c4ccccc4-c4ccccc4N(c4ccccc4)c4cccc5oc6ccccc6c45)cc2n3-c2ccccc2)cc1 ; c1ccc(-c2ccccc2-c2cccc3c2c2cc(-c4ccc(-c5ccccc5N(c5ccccc5)c5ccc6ccccc6c5)cc4)ccc2n3-c2ccccc2)cc1]

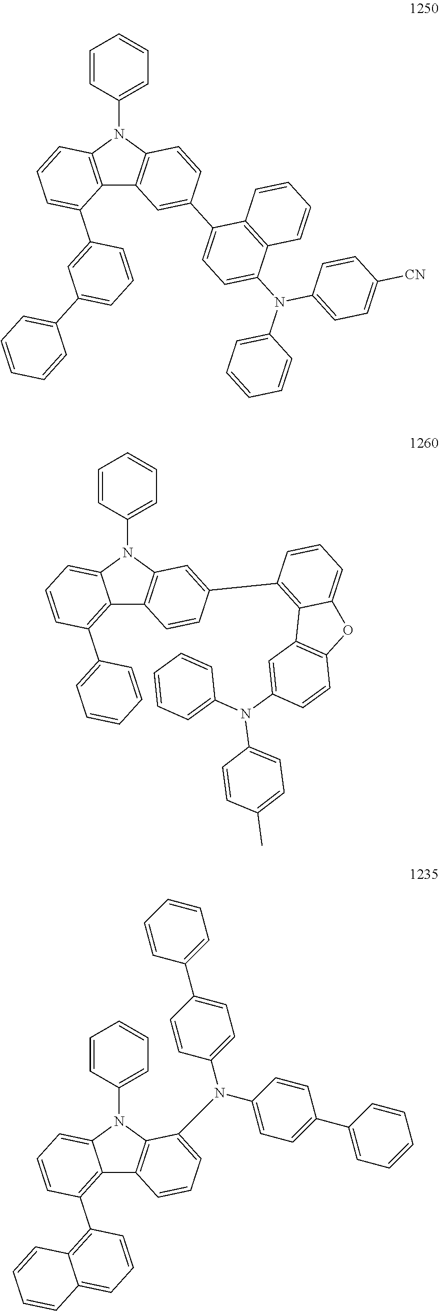 [SMILES: N#Cc1ccc(N(c2ccccc2)c2ccc(-c3ccc4c(c3)c3c(-c5cccc(-c6ccccc6)c5)cccc3n4-c3ccccc3)c3ccccc23)cc1 ; Cc1ccc(N(c2ccccc2)c2ccc3oc4cccc(-c5ccc6c7c(-c8ccccc8)cccc7n(-c7ccccc7)c6c5)c4c3c2)cc1 ; c1ccc(-c2ccc(N(c3ccc(-c4ccccc4)cc3)c3cccc4c5c(-c6cccc7ccccc67)cccc5n(-c5ccccc5)c34)cc2)cc1]

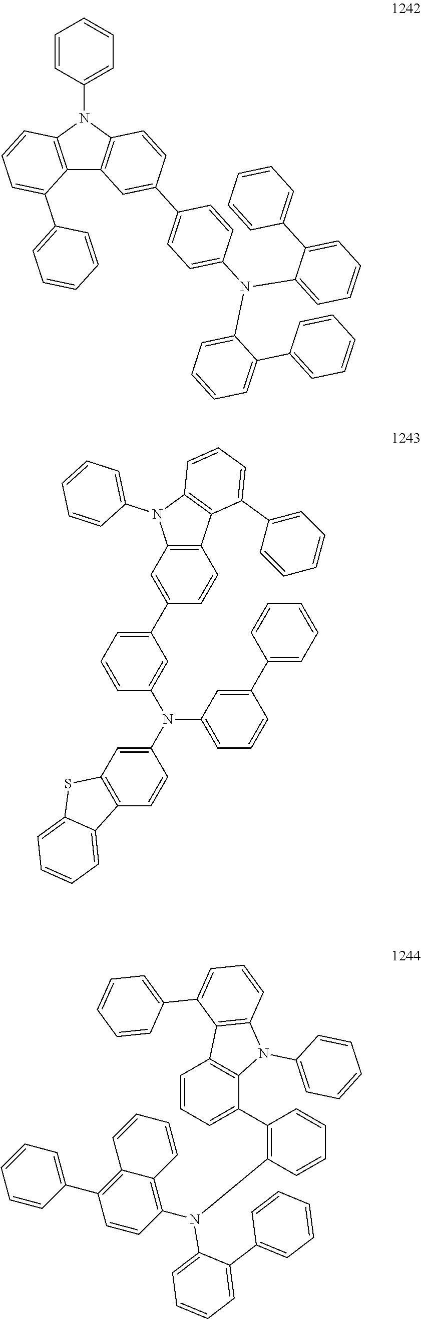 [SMILES: c1ccc(-c2ccccc2N(c2ccc(-c3ccc4c(c3)c3c(-c5ccccc5)cccc3n4-c3ccccc3)cc2)c2ccccc2-c2ccccc2)cc1 ; c1ccc(-c2cccc(N(c3cccc(-c4ccc5c6c(-c7ccccc7)cccc6n(-c6ccccc6)c5c4)c3)c3ccc4c(c3)sc3ccccc34)c2)cc1 ; c1ccc(-c2ccccc2N(c2ccccc2-c2cccc3c4c(-c5ccccc5)cccc4n(-c4ccccc4)c23)c2ccc(-c3ccccc3)c3ccccc23)cc1]

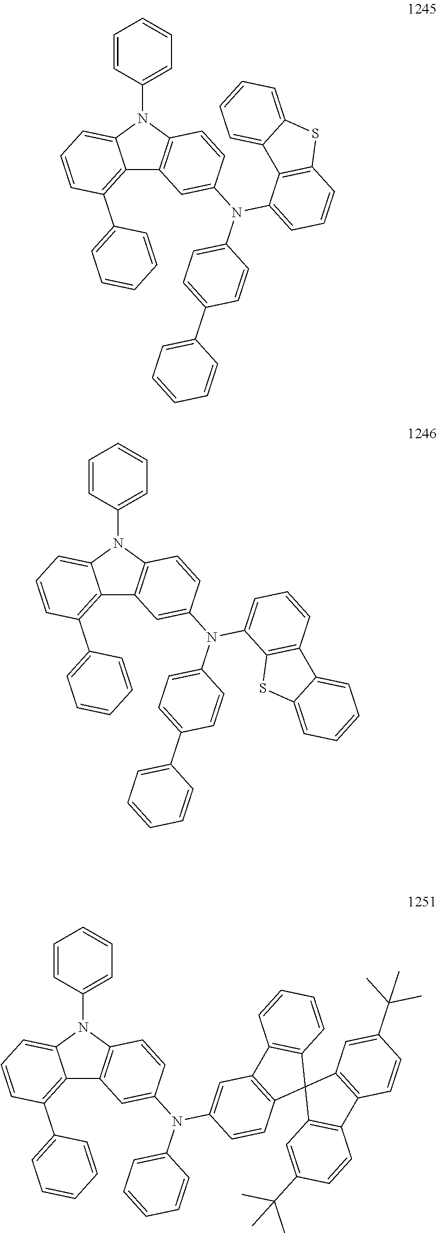 [SMILES: c1ccc(-c2ccc(N(c3ccc4c(c3)c3c(-c5ccccc5)cccc3n4-c3ccccc3)c3cccc4sc5ccccc5c34)cc2)cc1 ; c1ccc(-c2ccc(N(c3ccc4c(c3)c3c(-c5ccccc5)cccc3n4-c3ccccc3)c3cccc4c3sc3ccccc34)cc2)cc1 ; CC(C)(C)c1ccc2c(c1)C1(c3ccccc3-c3cc(N(c4ccccc4)c4ccc5c(c4)c4c(-c6ccccc6)cccc4n5-c4ccccc4)ccc31)c1cc(C(C)(C)C)ccc1-2]

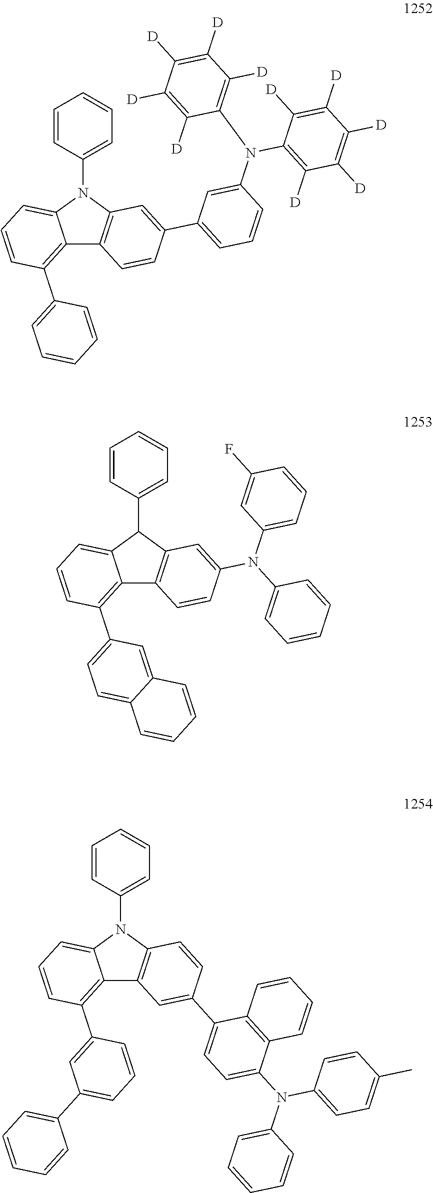 [SMILES: [2H]c1c([2H])c([2H])c(N(c2cccc(-c3ccc4c5c(-c6ccccc6)cccc5n(-c5ccccc5)c4c3)c2)c2c([2H])c([2H])c([2H])c([2H])c2[2H])c([2H])c1[2H] ; Fc1cccc(N(c2ccccc2)c2ccc3c(c2)C(c2ccccc2)c2cccc(-c4ccc5ccccc5c4)c2-3)c1 ; Cc1ccc(N(c2ccccc2)c2ccc(-c3ccc4c(c3)c3c(-c5cccc(-c6ccccc6)c5)cccc3n4-c3ccccc3)c3ccccc23)cc1]

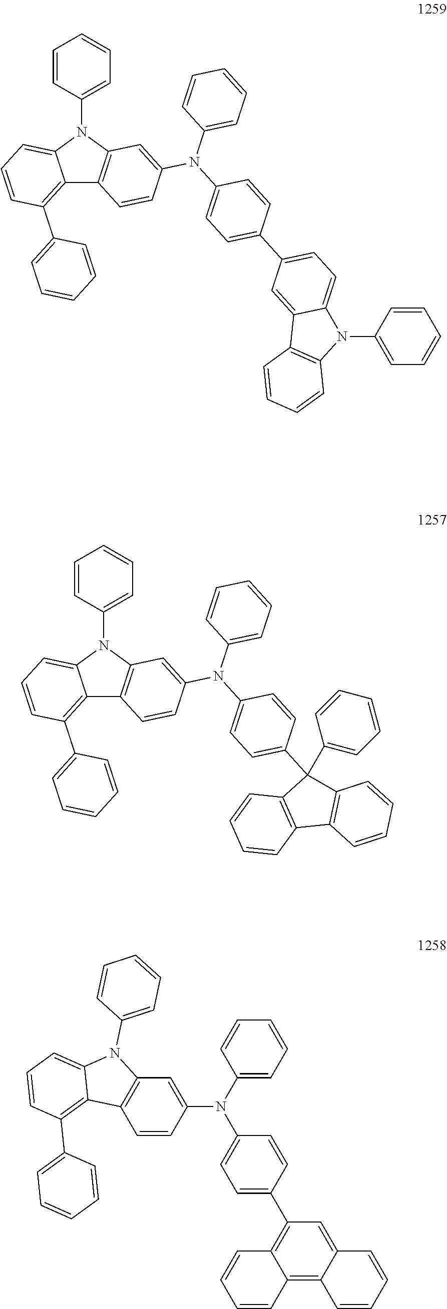 [SMILES: c1ccc(-c2cccc3c2c2ccc(N(c4ccccc4)c4ccc(-c5ccc6c(c5)c5ccccc5n6-c5ccccc5)cc4)cc2n3-c2ccccc2)cc1 ; c1ccc(-c2cccc3c2c2ccc(N(c4ccccc4)c4ccc(C5(c6ccccc6)c6ccccc6-c6ccccc65)cc4)cc2n3-c2ccccc2)cc1 ; c1ccc(-c2cccc3c2c2ccc(N(c4ccccc4)c4ccc(-c5cc6ccccc6c6ccccc56)cc4)cc2n3-c2ccccc2)cc1]

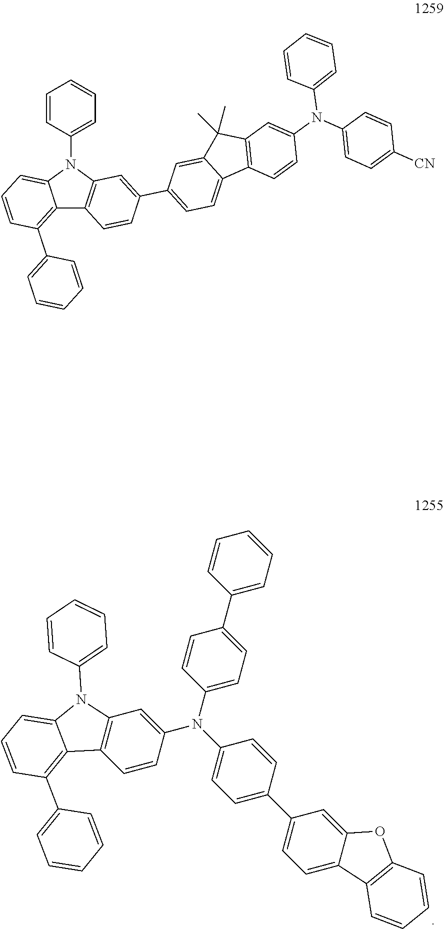 [SMILES: CC1(C)c2cc(-c3ccc4c5c(-c6ccccc6)cccc5n(-c5ccccc5)c4c3)ccc2-c2ccc(N(c3ccccc3)c3ccc(C#N)cc3)cc21 ; c1ccc(-c2ccc(N(c3ccc(-c4ccc5c(c4)oc4ccccc45)cc3)c3ccc4c5c(-c6ccccc6)cccc5n(-c5ccccc5)c4c3)cc2)cc1]

A synthetic method of the nitrogen-containing compound provided is not particularly limited in the present disclosure, and those skilled in the art can determine a suitable synthetic method according to the nitrogen-containing compound of the present disclosure in combination with preparation methods provided in Synthesis examples. In other words, the Synthesis examples of the present disclosure exemplarily provide methods for the preparation of the nitrogen-containing compounds, and the used raw materials may be commercially obtained or obtained by a method well known in the art. All nitrogen-containing compounds provided by the present disclosure can be obtained according to these exemplary preparation methods by those skilled in the art, and all specific preparation methods for the nitrogen-containing compounds are not described in detail here, which should not be understood by those skilled in the art as limiting the present disclosure.

In a second aspect, the present disclosure provides an electronic element, comprising an anode and a cathode which is arranged oppositely to the anode, and a functional layer disposed between the anode and the cathode, and the functional layer comprises the nitrogen-containing compound of the first aspect of the present disclosure.

The nitrogen-containing compound provided by the present disclosure has better hole transport properties and stability, and can be used as an electron blocking layer material of the organic electroluminescent device, and when used in an electronic element, the nitrogen-containing compound can be used for forming at least one organic film layer in the functional layer to improve the efficiency characteristics and service life characteristics of the electronic element.

Optionally, the functional layer comprises an electron blocking layer, and the electron blocking layer comprises the nitrogen-containing compound provided by the present disclosure. The electron blocking layer may be composed of the nitrogen-containing compound provided by the present disclosure or may be composed of the nitrogen-containing compound provided by the present disclosure together with other materials.

According to one embodiment, a hole transport layer is adjacent to the electron blocking layer, and is closer to the anode than the electron blocking layer.

Preferably, the electron blocking layer comprises the nitrogen-containing compound of the present disclosure and the organic electroluminescent device is a green light device.

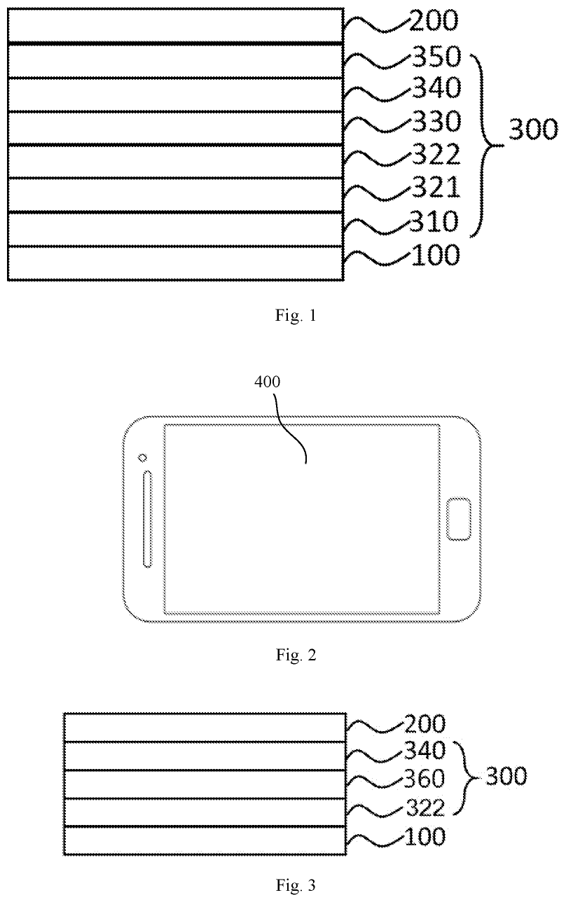

According to one embodiment, the electronic element may be an organic electroluminescent device. As shown in FIG. 1, the organic electroluminescent device may comprise an anode 100, a hole transport layer 321, an electron blocking layer 322, an organic light-emitting layer 330 as an energy conversion layer, an electron transport layer 340, and a cathode 200 which are sequentially stacked.

In the present disclosure, the anode 100 comprises an anode material, which is preferably a material having a large work function that facilitates hole injection into the functional layer. Specific examples of the anode material include, but are not limited to: metals such as nickel, platinum, vanadium, chromium, copper, zinc, and gold or their alloys; metal oxides such as zinc oxide, indium oxide, indium tin oxide (ITO), and indium zinc oxide (IZO); combined metals and oxides such as ZnO:Al or SnO₂:Sb; or conducting polymers such as poly(3-methylthiophene), poly[3,4-(ethylene-1,2-dioxy)thiophene] (PEDT), polypyrrole, and polyaniline. A transparent electrode comprising indium tin oxide (ITO) as the anode is preferably included.

According to one embodiment, the hole transport layer 321 may comprise an inorganic doping material to improve the hole transport ability of the hole transport layer 321.

Optionally, the hole transport layer 321 comprises one or more hole transport materials, and the hole transport materials may be selected from a carbazole polymer, carbazole-linked triarylamine compounds, or other types of compounds, which are not specially limited in the present disclosure. For example, the hole transport layer 321 may be comprised of a compound NPB.

According to one more specific embodiment, the organic electroluminescent device is a green light device, and the electron blocking layer 322 comprises the nitrogen-containing compound of the present disclosure.

Optionally, the organic light-emitting layer 330 may be composed of a single light-emitting material, and may also comprise a host material and a guest material. In one specific embodiment, the organic light-emitting layer 330 is composed of the host material and the guest material, and holes injected into the organic light-emitting layer 330 and electrons injected into the organic light-emitting layer 330 can be recombined in the organic light-emitting layer 330 to form excitons, the excitons transfer energy to the host material, and the host material transfers energy to the guest material, thus enabling the guest material to emit light.

The host material of the organic light-emitting layer 330 may be a carbazole derivative, a metal chelate compound, a bis-styryl derivative, an aromatic amine derivative, a dibenzofuran derivative, or other types of materials, which is not specially limited in the present disclosure. In one embodiment of the present disclosure, the host material of the organic light-emitting layer 330 may be GH-n1 and GH-n2.

The guest material of the organic light-emitting layer 330 may be a compound having a condensed aryl ring or its derivative, a compound having a heteroaryl ring or its derivative, an aromatic amine derivative, a metal complex, or other materials, which is not specially limited in the present disclosure. In one embodiment of the present disclosure, the guest material of the organic light-emitting layer 330 can be Ir(ppy)₃.

The electron transport layer 340 may be of a single-layer structure or a multi-layer structure, and may comprise one or more electron transport materials, and the electron transport materials may be selected from, but are not limited to, a benzimidazole derivative, an oxadiazole derivative, a quinoxaline derivative, or other electron transport materials. In one embodiment of the present disclosure, the electron transport layer 340 may be composed of ET-06 and LiQ.

In the present disclosure, specific structures of compounds such as GH-n1, GH-n2 and ET-06 are shown in the Examples below, which will not be repeated here.

In the present disclosure, the cathode 200 may comprise a cathode material, which is a material having a small work function that facilitates electron injection into the functional layer. Specific embodiments of the cathode material include, but are not limited to, metals such as magnesium, calcium, sodium, potassium, titanium, indium, yttrium, lithium, gadolinium, aluminum, silver, tin, and lead or their alloys; or multilayer materials such as LiF/Al, Liq/Al, LiO₂/Al, LiF/Ca, LiF/Al, and BaF₂/Ca. A metal electrode comprising magnesium and silver as the cathode is preferably included.

Optionally, as shown in FIG. 1, a hole injection layer 310 may also be arranged between the anode 100 and the hole transport layer 321 to enhance the ability to inject holes into the hole transport layer 321. The hole injection layer 310 may be selected from a benzidine derivative, a starburst arylamine compound, a phthalocyanine derivative or other materials, which is not specially limited in the present disclosure. For example, the hole injection layer 310 may be composed of F4-TCNQ.

Optionally, as shown in FIG. 1, an electron injection layer 350 may also be arranged between the cathode 200 and the electron transport layer 340 to enhance the ability to inject electrons into the electron transport layer 340. The electron injection layer 350 may include an inorganic material such as an alkali metal sulfide and an alkali metal halide, or may include a complex of an alkali metal and an organic substance. For example, the electron injection layer 350 may comprise LiQ.

According to another embodiment, the electronic element may be a photoelectric conversion device. As shown in FIG. 3, the photoelectric conversion device may comprise an anode 100 and a cathode 200 which is arranged oppositely to the anode, and a functional layer 300 disposed between the anode 100 and the cathode 200; and the functional layer 300 comprises the nitrogen-containing compound provided by the present disclosure.

According to an exemplary embodiment, as shown in FIG. 3, the functional layer 300 comprises an electron blocking layer 322 that comprises the nitrogen-containing compound of the present disclosure. The electron blocking layer 322 may be composed of the nitrogen-containing compound provided by the present disclosure, or may be composed of the nitrogen-containing compound provided by the present disclosure together with other materials.

According to one specific embodiment, as shown in FIG. 3, the photoelectric conversion device may comprise an anode 100, an electron blocking layer 322, a photoelectric conversion layer 360, an electron transport layer 340, and a cathode 200 which are sequentially stacked.

Optionally, the photoelectric conversion device can be a solar cell, in particular an organic thin-film solar cell. For example, in one embodiment of the present disclosure, the solar cell may comprise an anode, a hole transport layer, a photoelectric conversion layer, an electron transport layer, and a cathode which are sequentially stacked.

In a third aspect, the present disclosure provides an electronic apparatus, comprising the electronic element of the second aspect of the present disclosure.

According to one embodiment, as shown in FIG. 2, the electronic apparatus is a first electronic apparatus 400 including the organic electroluminescent device described above. The first electronic apparatus 400 may be, for example, a display device, a lighting device, an optical communication device, or other types of electronic devices, and may include, for example, but is not limited to, a computer screen, a mobile phone screen, a television, electronic paper, an emergency lighting lamp, an optical module, and the like.

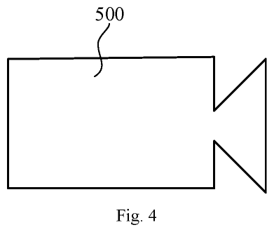

According to another embodiment, as shown in FIG. 4, the electronic apparatus is a second electronic apparatus 500 including the photoelectric conversion device described above. The second electronic apparatus 500 may be, for example, a solar power plant, a light detector, a fingerprint recognition device, an optical module, a CCD camera, or other types of electronic devices.

Compounds of which synthetic methods are not mentioned in the present disclosure are raw material products obtained by commercial routes.

In the following, a number of specific embodiments are exemplarily provided to further explain and illustrate the present disclosure. However, the following examples are merely illustrative of the present disclosure and are not intended to limit the present disclosure.

### Synthesis Examples

### Synthesis of Intermediates

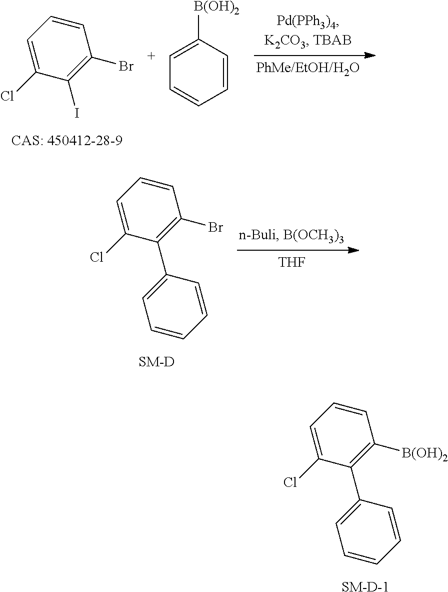

1-Bromo-2-iodo-3-chlorobenzene (50.0 g, 157.5 mmol), phenylboronic acid (19.2 g, 157.5 mmol), tetrakis(triphenylphosphine)palladium (9.1 g, 7.8 mmol), tetrabutylammonium bromide (2.5 g, 7.8 mmol), potassium carbonate (65.2 g, 472.6 mmol), toluene (400 mL), ethanol (200 mL), and deionized water (100 mL) were added into a dry round bottom flask replaced with nitrogen, and the reaction solution was heated to 75° C. under stirring for 8 h; the reaction mixture was then cooled to room temperature, deionized water (200 mL) was added, stirring was performed for 15 min, an organic phase was separated, and dried over anhydrous magnesium sulfate, and a solvent was removed under reduced pressure; and the obtained crude product was purified by silica gel column chromatography using dichloromethane/n-heptane (a volume ratio of 1:3) as a mobile phase to obtain an intermediate SM-D (35.8 g, yield: 85%).

SM-D (35.8 g, 133.8 mmol) and tetrahydrofuran (400 mL) were added to a dry round bottom flask replaced with nitrogen, after cooling to −78° C., n-butyllithium (12.8 g, 200.7 mmol) was added dropwise, after the dropwise addition was complete, kept temperature at −78° C. for 30 min, then trimethyl borate (41.7 g, 401.4 mmol) was added dropwise, and after the dropwise addition was complete, kept temperature at −78° C. for 30 min. After heating to room temperature and stirring for 12 h, an aqueous hydrochloric acid solution was added to the reaction solution to adjust a pH to be neutral. The resulting reaction solution was filtered to obtain a crude product, and the crude product was recrystallized by using n-heptane (600 mL) to give an intermediate SM-D-1 (19.6 g, yield: 63%).

With reference to the synthesis method of the intermediate SM-D, intermediates shown in Table 1 below were synthesized by using a reactant Q in the following table instead of phenylboronic acid:

TABLE 1

| Intermediate | Reactant Q | Structure | Yield (%) |
|---|---|---|---|
| SM-2 | 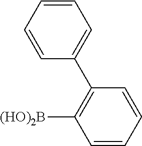 [SMILES: OB(O)c1ccccc1-c1ccccc1] | 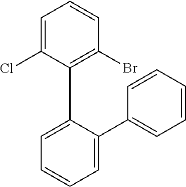 [SMILES: Clc1cccc(Br)c1-c1ccccc1-c1ccccc1] | 83 |
| SM-3 | 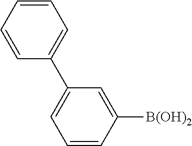 [SMILES: OB(O)c1cccc(-c2ccccc2)c1] | 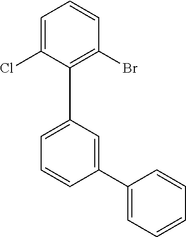 [SMILES: Clc1cccc(Br)c1-c1cccc(-c2ccccc2)c1] | 82 |
| SM-4 | 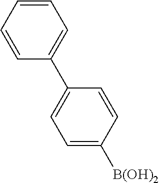 [SMILES: OB(O)c1ccc(-c2ccccc2)cc1] | 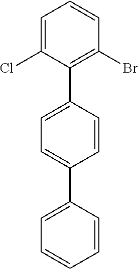 [SMILES: Clc1cccc(Br)c1-c1ccc(-c2ccccc2)cc1] | 84 |
| SM-5 | 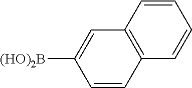 [SMILES: OB(O)c1ccc2ccccc2c1] | 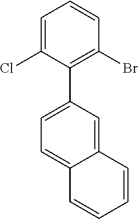 [SMILES: Clc1cccc(Br)c1-c1ccc2ccccc2c1] | 83 |
| SM-6 | 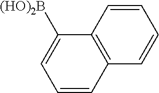 [SMILES: OB(O)c1cccc2ccccc12] | 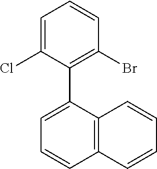 [SMILES: Clc1cccc(Br)c1-c1cccc2ccccc12] | 81 |

With reference to the synthesis method of the intermediate SM-D, intermediates shown in Table 2 below were synthesized by using a reactant N in the following table instead of 1-bromo-2-iodo-3-chlorobenzene and a reactant M in the following table instead of phenylboronic acid:

TABLE 2

| | | | | Yield |
|---|---|---|---|---|
| Intermediate | Reactant N | Reactant M | Structure | (%) |
| SM-7 | 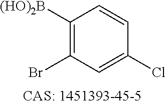 [SMILES: OB(O)c1ccc(Cl)cc1Br] | 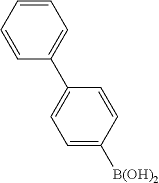 [SMILES: OB(O)c1ccc(-c2ccccc2)cc1] | 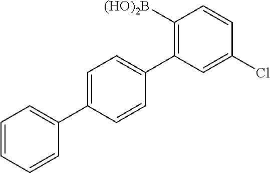 [SMILES: OB(O)c1ccc(Cl)cc1-c1ccc(-c2ccccc2)cc1] | 80 |
| SM-8 | | 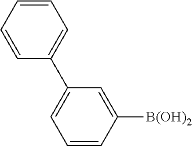 [SMILES: OB(O)c1cccc(-c2ccccc2)c1] | 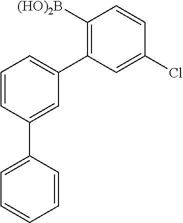 [SMILES: OB(O)c1ccc(Cl)cc1-c1cccc(-c2ccccc2)c1] | 79 |
| SM-9 | | 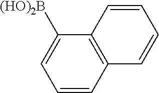 [SMILES: OB(O)c1cccc2ccccc12] | 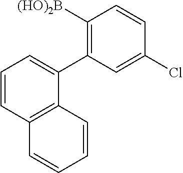 [SMILES: OB(O)c1ccc(Cl)cc1-c1cccc2ccccc12] | 81 |
| SM-10 | 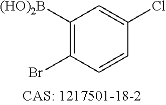 [SMILES: OB(O)c1cc(Cl)ccc1Br] | 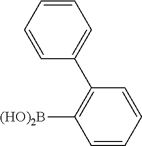 [SMILES: OB(O)c1ccccc1-c1ccccc1] | 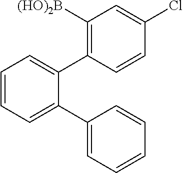 [SMILES: OB(O)c1cc(Cl)ccc1-c1ccccc1-c1ccccc1] | 80 |
| SM-11 | 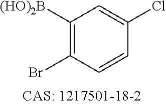 [SMILES: OB(O)c1cc(Cl)ccc1Br] | 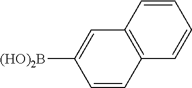 [SMILES: OB(O)c1ccc2ccccc2c1] | 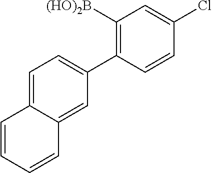 [SMILES: OB(O)c1cc(Cl)ccc1-c1ccc2ccccc2c1] | 78 |

With reference to the synthesis method of the intermediate SM-D-1, intermediates shown in Table 3 below were synthesized by using (reactants) intermediates in the following table instead of SM-D:

TABLE 3

| | (Reactant) | | Yield |
|---|---|---|---|
| Intermediate | intermediate | Structure | (%) |
| SM-D-2 | 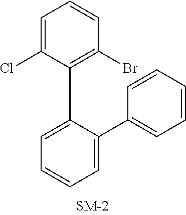 [SMILES: Clc1cccc(Br)c1-c1ccccc1-c1ccccc1] | 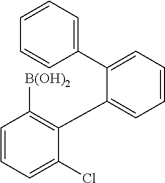 [SMILES: OB(O)c1cccc(Cl)c1-c1ccccc1-c1ccccc1] | 62 |
| SM-D-3 | 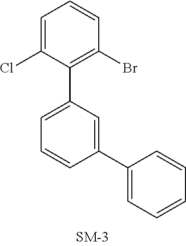 [SMILES: Clc1cccc(Br)c1-c1cccc(-c2ccccc2)c1] | 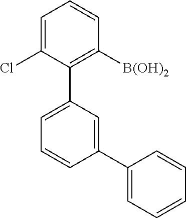 [SMILES: OB(O)c1cccc(Cl)c1-c1cccc(-c2ccccc2)c1] | 61 |
| SM-D-4 | 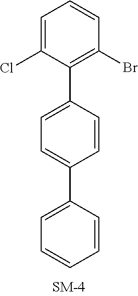 [SMILES: Clc1cccc(Br)c1-c1ccc(-c2ccccc2)cc1] | 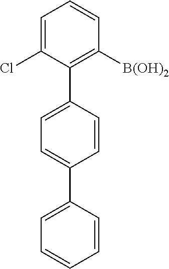 [SMILES: OB(O)c1cccc(Cl)c1-c1ccc(-c2ccccc2)cc1] | 63 |
| SM-D-5 | 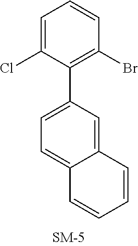 [SMILES: Clc1cccc(Br)c1-c1ccc2ccccc2c1] | 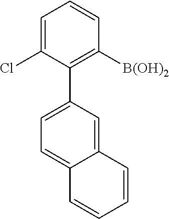 [SMILES: OB(O)c1cccc(Cl)c1-c1ccc2ccccc2c1] | 64 |
| SM-D-6 | 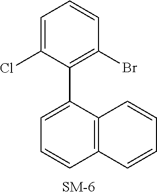 [SMILES: Clc1cccc(Br)c1-c1cccc2ccccc12] | 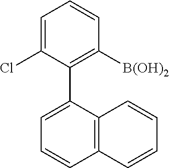 [SMILES: OB(O)c1cccc(Cl)c1-c1cccc2ccccc12] | 63 |

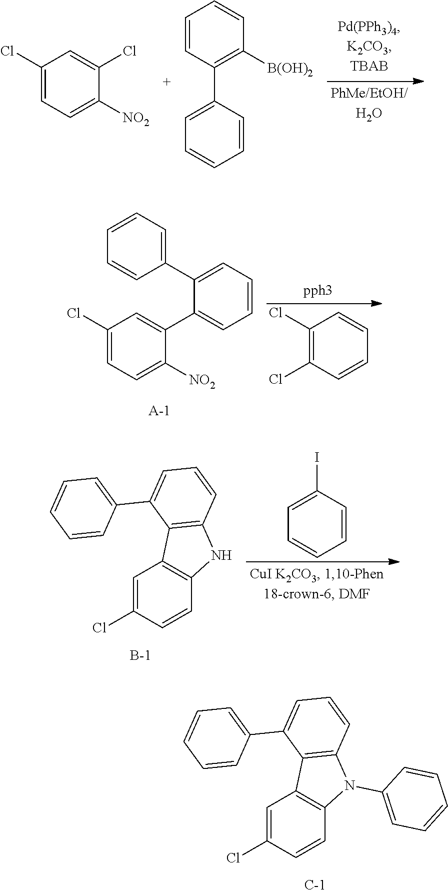

Biphenyl-2-boronic acid (51.6 g, 260.4 mmol), 2,4-dichloronitrobenzene (50.0 g, 260.4 mmol), tetrakis(triphenylphosphine)palladium (15.0 g, 13.0 mmol), tetrabutylammonium bromide (4.2 g, 13.0 mmol), potassium carbonate (107.9 g, 781.3 mmol), toluene (400 mL), ethanol (200 mL), and deionized water (100 mL) were added into a dry round bottom flask replaced with nitrogen, and the reaction solution was heated to 75° C. under stirring for 8 h; the reaction mixture was then cooled to room temperature, deionized water (200 mL) was added, stirring was performed for 15 min, an organic phase was separated, and dried over anhydrous magnesium sulfate, and a solvent was removed under reduced pressure; and the obtained crude product was purified by silica gel column chromatography using dichloromethane/n-heptane (a volume ratio of 1:3) as a mobile phase to obtain an intermediate A-1 (56.5 g; yield: 70%).

The intermediate A-1 (56.5 g, 182.4 mmol), triphenylphosphine (119.6 g, 456.0 mmol), and ortho-dichlorobenzene (400 mL) were added into a dry round bottom flask replaced with nitrogen, the reaction solution was heated to 160° C. under stirring, and subjected to a reaction for 6 h; and silica gel was then added to the reaction solution to volatilize liquid, followed by silica gel column chromatography using dichloromethane/n-heptane (a volume ratio of 1:3) as a mobile phase to obtain an intermediate B-1 (30.4 g, yield: 60%).

The intermediate B-1 (30.4 g, 109.5 mmol), iodobenzene (22.7 g, 111.6 mmol), cuprous iodide (4.2 g, 21.8 mmol), potassium carbonate (33.3 g, 240.8 mmol), 1,10-phenanthroline (7.9 g, 43.8 mmol), 18-crown-6 (2.9 g, 10.9 mmol), and N,N-dimethylformamide (300 mL) were added into a dry round bottom flask replaced with nitrogen, and heated to 160° C. under stirring for 8 h; the reaction mixture was then cooled to room temperature, deionized water (200 mL) was added, stirring was performed for 15 min, an organic phase was separated, and dried over anhydrous magnesium sulfate, and a solvent was removed under reduced pressure; and the obtained crude product was purified by silica gel column chromatography using dichloromethane/n-heptane (a volume ratio of 1:3) as a mobile phase to obtain an intermediate C-1 (30.9 g, yield: 80%).

With reference to the same method as that for the synthesis of the intermediate A-1, intermediates in Table 4 below were synthesized by using a reactant A in the following table instead of 2,4-dichloronitrobenzene and SM-D-X in the following table instead of biphenyl-2-boronic acid:

TABLE 4

| Intermediate | Reactant A | SM-D-X | Structure | Yield (%) |
|---|---|---|---|---|
| A-2 | 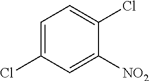 [SMILES: O=[N+]([O-])c1cc(Cl)ccc1Cl] | | 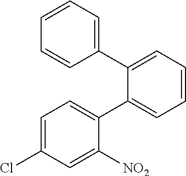 [SMILES: O=[N+]([O-])c1cc(Cl)ccc1-c1ccccc1-c1ccccc1] | 69 |
| A-3 | 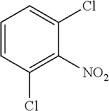 [SMILES: O=[N+]([O-])c1c(Cl)cccc1Cl] | 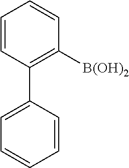 | 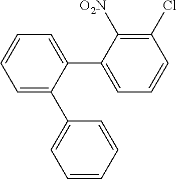 [SMILES: O=[N+]([O-])c1c(Cl)cccc1-c1ccccc1-c1ccccc1] | 65 |
| A-13 | 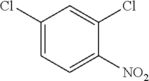 [SMILES: O=[N+]([O-])c1ccc(Cl)cc1Cl] | 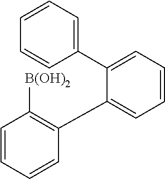 | 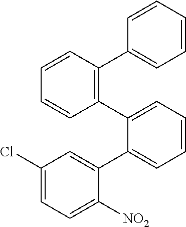 [SMILES: O=[N+]([O-])c1ccc(Cl)cc1-c1ccccc1-c1ccccc1-c1ccccc1] | 63 |
| A-14 | 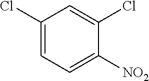 [SMILES: O=[N+]([O-])c1ccc(Cl)cc1Cl] | 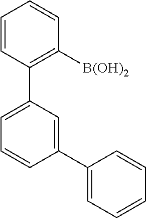 | 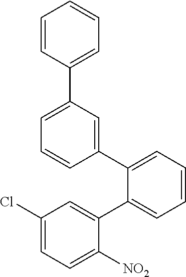 [SMILES: O=[N+]([O-])c1ccc(Cl)cc1-c1ccccc1-c1cccc(-c2ccccc2)c1] | 68 |
| A-15 | 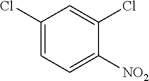 [SMILES: O=[N+]([O-])c1ccc(Cl)cc1Cl] | 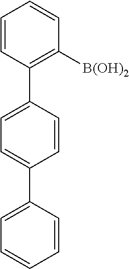 | 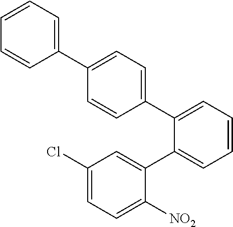 [SMILES: O=[N+]([O-])c1ccc(Cl)cc1-c1ccccc1-c1ccc(-c2ccccc2)cc1] | 66 |
| A-16 | 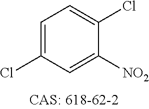 [SMILES: O=[N+]([O-])c1cc(Cl)ccc1Cl] | 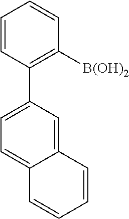 | 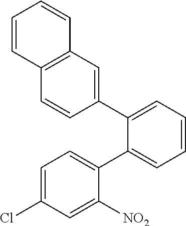 [SMILES: O=[N+]([O-])c1cc(Cl)ccc1-c1ccccc1-c1ccc2ccccc2c1] | 65 |
| A-17 | 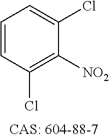 [SMILES: O=[N+]([O-])c1c(Cl)cccc1Cl] | 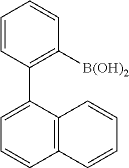 | 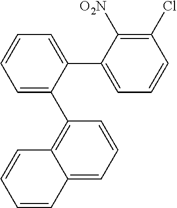 [SMILES: O=[N+]([O-])c1c(Cl)cccc1-c1ccccc1-c1cccc2ccccc12] | 64 |

With reference to the synthesis method of the intermediate B-1, intermediates in Table 5 below were synthesized by using a reactant B in the following table instead of the intermediate A-1:

TABLE 5

| Intermediate | Reactant B | Structure | Yield (%) |
|---|---|---|---|
| B-2 | 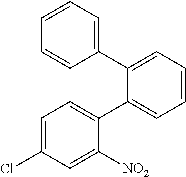 [SMILES: O=[N+]([O-])c1cc(Cl)ccc1-c1ccccc1-c1ccccc1] | 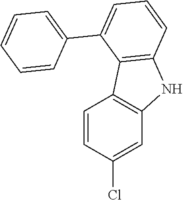 [SMILES: Clc1ccc2c(c1)[nH]c1cccc(-c3ccccc3)c12] | 59 |
| B-3 | 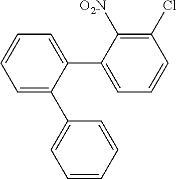 [SMILES: O=[N+]([O-])c1c(Cl)cccc1-c1ccccc1-c1ccccc1] | 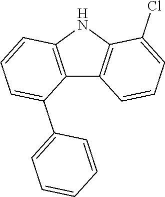 [SMILES: Clc1cccc2c1[nH]c1cccc(-c3ccccc3)c12] | 58 |
| B-13 | 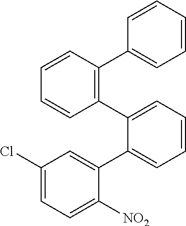 [SMILES: O=[N+]([O-])c1ccc(Cl)cc1-c1ccccc1-c1ccccc1-c1ccccc1] | 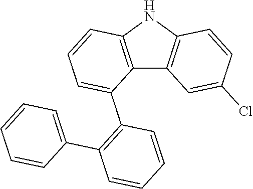 [SMILES: Clc1ccc2[nH]c3cccc(-c4ccccc4-c4ccccc4)c3c2c1] | 59 |
| B-14 | 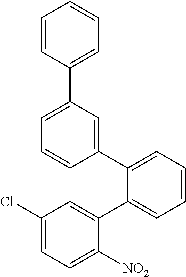 [SMILES: O=[N+]([O-])c1ccc(Cl)cc1-c1ccccc1-c1cccc(-c2ccccc2)c1] | 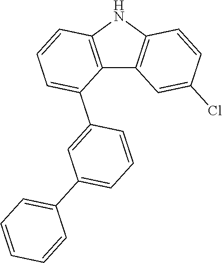 [SMILES: Clc1ccc2[nH]c3cccc(-c4cccc(-c5ccccc5)c4)c3c2c1] | 58 |
| B-15 | 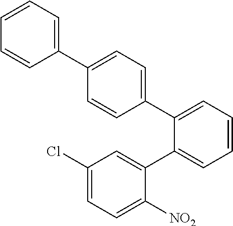 [SMILES: O=[N+]([O-])c1ccc(Cl)cc1-c1ccccc1-c1ccc(-c2ccccc2)cc1] | 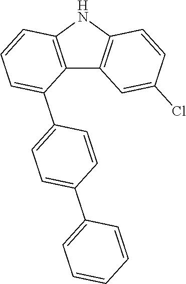 [SMILES: Clc1ccc2[nH]c3cccc(-c4ccc(-c5ccccc5)cc4)c3c2c1] | 57 |
| B-16 | 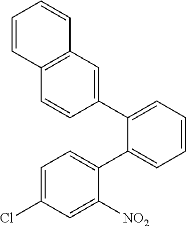 [SMILES: O=[N+]([O-])c1cc(Cl)ccc1-c1ccccc1-c1ccc2ccccc2c1] | 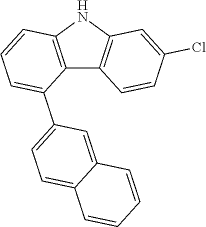 [SMILES: Clc1ccc2c(c1)[nH]c1cccc(-c3ccc4ccccc4c3)c12] | 58 |
| B-17 | 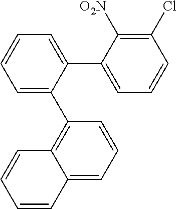 [SMILES: O=[N+]([O-])c1c(Cl)cccc1-c1ccccc1-c1cccc2ccccc12] | 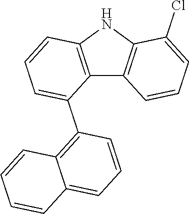 [SMILES: Clc1cccc2c1[nH]c1cccc(-c3cccc4ccccc34)c12] | 59 |

With reference to the same method as that for the synthesis of the intermediate C-1, intermediates in Table 6 below were synthesized by using a reactant C in the following table instead of the intermediate B-1:

TABLE 6

| Intermediate | Reactant C | Structure | Yield (%) |
|---|---|---|---|
| C-2 | 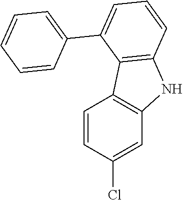 [SMILES: Clc1ccc2c(c1)[nH]c1cccc(-c3ccccc3)c12] | 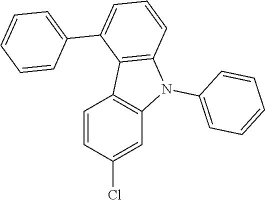 [SMILES: Clc1ccc2c3c(-c4ccccc4)cccc3n(-c3ccccc3)c2c1] | 80 |
| C-3 | 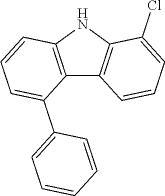 [SMILES: Clc1cccc2c1[nH]c1cccc(-c3ccccc3)c12] | 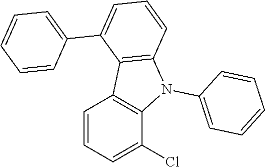 [SMILES: Clc1cccc2c3c(-c4ccccc4)cccc3n(-c3ccccc3)c12] | 81 |
| C-13 | 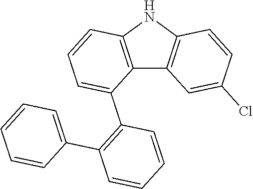 [SMILES: Clc1ccc2[nH]c3cccc(-c4ccccc4-c4ccccc4)c3c2c1] | 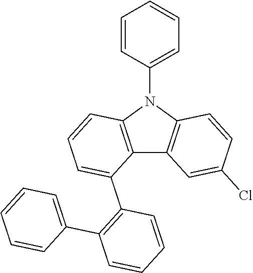 [SMILES: Clc1ccc2c(c1)c1c(-c3ccccc3-c3ccccc3)cccc1n2-c1ccccc1] | 79 |
| C-14 | 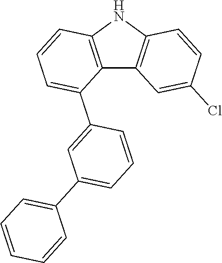 [SMILES: Clc1ccc2[nH]c3cccc(-c4cccc(-c5ccccc5)c4)c3c2c1] | 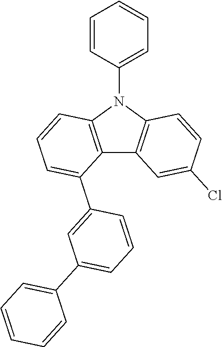 [SMILES: Clc1ccc2c(c1)c1c(-c3cccc(-c4ccccc4)c3)cccc1n2-c1ccccc1] | 80 |
| C-15 | 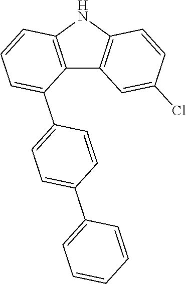 [SMILES: Clc1ccc2[nH]c3cccc(-c4ccc(-c5ccccc5)cc4)c3c2c1] | 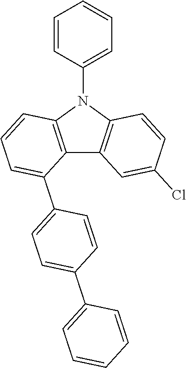 [SMILES: Clc1ccc2c(c1)c1c(-c3ccc(-c4ccccc4)cc3)cccc1n2-c1ccccc1] | 75 |
| C-16 | 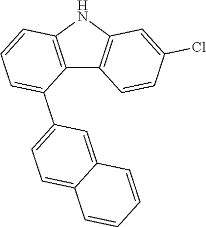 [SMILES: Clc1ccc2c(c1)[nH]c1cccc(-c3ccc4ccccc4c3)c12] | 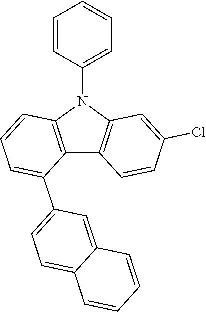 [SMILES: Clc1ccc2c3c(-c4ccc5ccccc5c4)cccc3n(-c3ccccc3)c2c1] | 76 |
| C-17 | 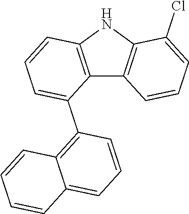 [SMILES: Clc1cccc2c1[nH]c1cccc(-c3cccc4ccccc34)c12] | 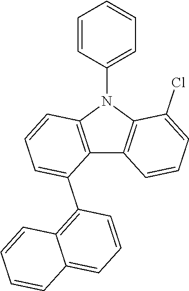 [SMILES: Clc1cccc2c3c(-c4cccc5ccccc45)cccc3n(-c3ccccc3)c12] | 75 |

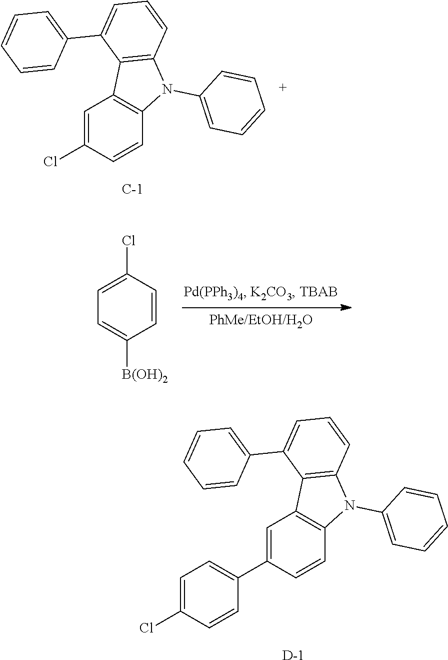

p-chlorophenylboronic acid (2.19 g, 14.13 mmol), the intermediate C-1 (5.0 g, 14.13 mmol), tetrakis(triphenylphosphine)palladium (0.82 g, 0.70 mmol), tetrabutylammonium bromide (0.23 g, 0.71 mmol), potassium carbonate (3.90 g, 28.26 mmol), toluene (40 mL), ethanol (20 mL), and deionized water (10 mL) were added into a dry round bottom flask replaced with nitrogen, and the reaction solution was heated to 75° C. under stirring for 8 h; the reaction mixture was then cooled to room temperature, deionized water (100 mL) was added, stirring was performed for 15 min, an organic phase was separated, and dried over anhydrous magnesium sulfate, and a solvent was removed under reduced pressure; and the obtained crude product was purified by silica gel column chromatography using dichloromethane/n-heptane (a volume ratio of 1:3) as a mobile phase to obtain an intermediate D-1 (4.6 g, yield: 75%).

Intermediates in Table 7 below were synthesized by the same method as that for the synthesis of the intermediate D-1 by using a reactant D in the following table instead of the intermediate C-1 and SM-D in the following table instead of p-chlorophenylboronic acid:

TABLE 7

| Inter- | | | | Yield |
|---|---|---|---|---|
| mediate | Reactant D | SM-D | Structure | (%) |
| D-2 | 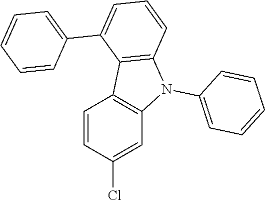 [SMILES: Clc1ccc2c3c(-c4ccccc4)cccc3n(-c3ccccc3)c2c1] | 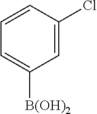 | 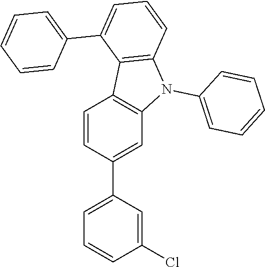 [SMILES: Clc1cccc(-c2ccc3c4c(-c5ccccc5)cccc4n(-c4ccccc4)c3c2)c1] | 71 |
| D-3 | 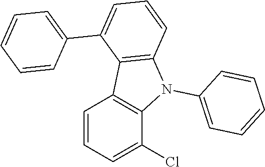 [SMILES: Clc1cccc2c3c(-c4ccccc4)cccc3n(-c3ccccc3)c12] | 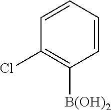 | 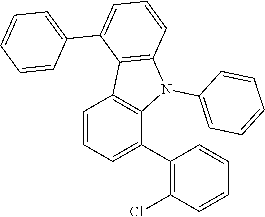 [SMILES: Clc1ccccc1-c1cccc2c3c(-c4ccccc4)cccc3n(-c3ccccc3)c12] | 70 |
| D-8 | 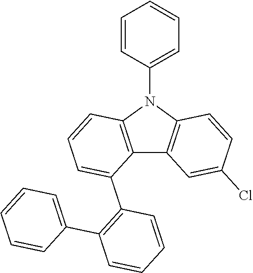 [SMILES: Clc1ccc2c(c1)c1c(-c3ccccc3-c3ccccc3)cccc1n2-c1ccccc1] | 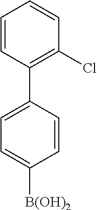 | 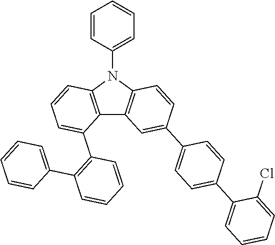 [SMILES: Clc1ccccc1-c1ccc(-c2ccc3c(c2)c2c(-c4ccccc4-c4ccccc4)cccc2n3-c2ccccc2)cc1] | 72 |
| D-9 | 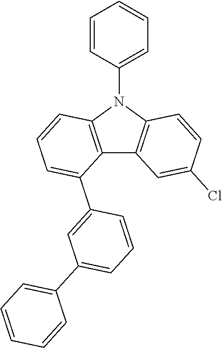 [SMILES: Clc1ccc2c(c1)c1c(-c3cccc(-c4ccccc4)c3)cccc1n2-c1ccccc1] | 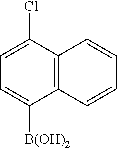 | 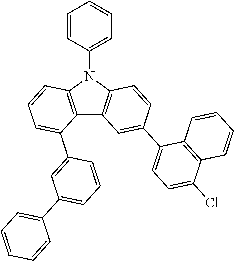 [SMILES: Clc1ccc(-c2ccc3c(c2)c2c(-c4cccc(-c5ccccc5)c4)cccc2n3-c2ccccc2)c2ccccc12] | 70 |
| D-10 | 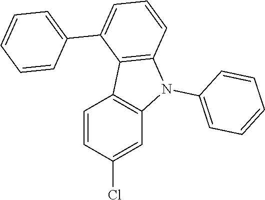 [SMILES: Clc1ccc2c3c(-c4ccccc4)cccc3n(-c3ccccc3)c2c1] | 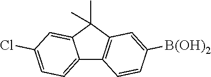 | 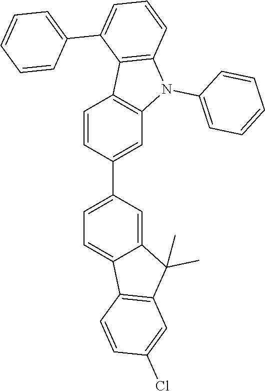 [SMILES: CC1(C)c2cc(Cl)ccc2-c2ccc(-c3ccc4c5c(-c6ccccc6)cccc5n(-c5ccccc5)c4c3)cc21] | 68 |
| D-11 | | 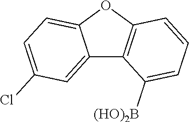 | 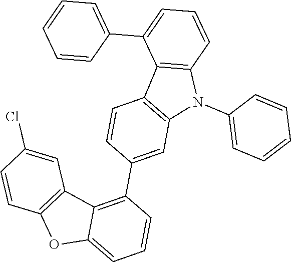 [SMILES: Clc1ccc2oc3cccc(-c4ccc5c6c(-c7ccccc7)cccc6n(-c6ccccc6)c5c4)c3c2c1] | 67 |

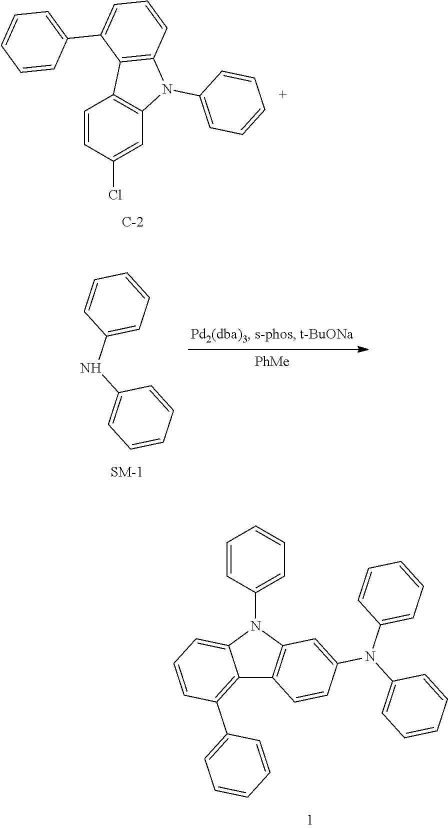

The intermediate C-2 (10 g, 14.1 mmol), SM-1 (diphenylamine) (2.4 g, 14.1 mmol), tris(dibenzylideneacetone)dipalladium (0.1 g, 0.1 mmol), 2-dicyclohexylphosphino-2′,6′-dimethoxy-biphenyl (0.1 g, 0.3 mmol), sodium tert-butoxide (2.0 g, 21.2 mmol) and a toluene solvent (100 mL) were added into a reaction flask, and the reaction solution was heated to 110° C. under nitrogen protection, and stirred under heating and refluxing for 8 h. After the reaction solution was cooled to room temperature, the reaction solution was extracted with dichloromethane (50 mL) and water (50 mL), and washed with water for 3 times, an organic layer was dried over anhydrous magnesium sulfate, and filtered, the obtained filtrate was allowed to pass through a short silica gel column, a solvent was removed under reduced pressure, and a crude product was purified by recrystallization using a dichloromethane/n-heptane system (1:3) to obtain a compound 1 (4.8 g, yield: 70%).

With reference to the synthesis method of the compound 1, compounds in Table 8 below were synthesized by using SM-Y in the following table instead of SM-1 (diphenylamine) and intermediates C-X/D-X in the following table instead of the intermediate C-1:

TABLE 8

| Intermediate C- | | | Yield |
|---|---|---|---|
| X/D-X | SM-Y | Compound No. | (%) |
| 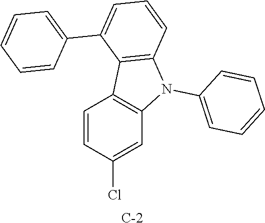 | 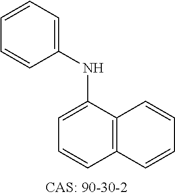 | 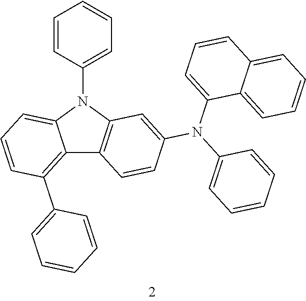 [SMILES: c1ccc(-c2cccc3c2c2ccc(N(c4ccccc4)c4cccc5ccccc45)cc2n3-c2ccccc2)cc1] | 71 |
| | 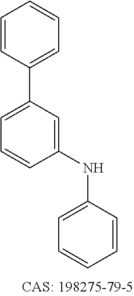 | 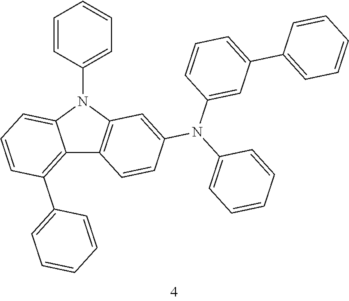 [SMILES: c1ccc(-c2cccc(N(c3ccccc3)c3ccc4c5c(-c6ccccc6)cccc5n(-c5ccccc5)c4c3)c2)cc1] | 72 |
| | 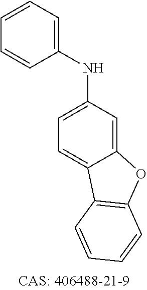 | 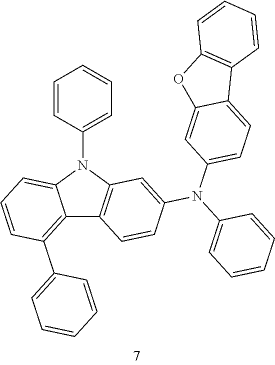 [SMILES: c1ccc(-c2cccc3c2c2ccc(N(c4ccccc4)c4ccc5c(c4)oc4ccccc45)cc2n3-c2ccccc2)cc1] | 71 |
| | 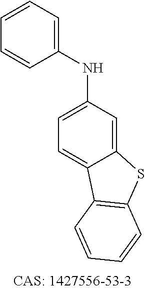 | 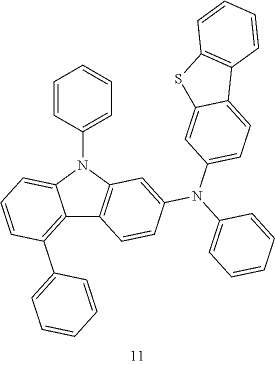 [SMILES: c1ccc(-c2cccc3c2c2ccc(N(c4ccccc4)c4ccc5c(c4)sc4ccccc45)cc2n3-c2ccccc2)cc1] | 70 |
| 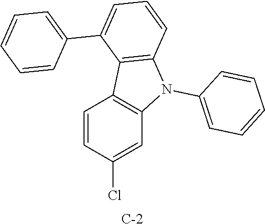 | 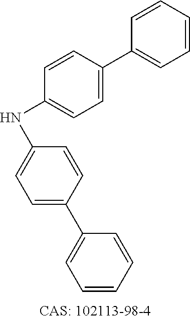 | 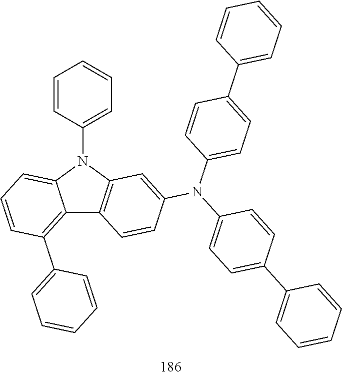 [SMILES: c1ccc(-c2ccc(N(c3ccc(-c4ccccc4)cc3)c3ccc4c5c(-c6ccccc6)cccc5n(-c5ccccc5)c4c3)cc2)cc1] | 71 |
| | 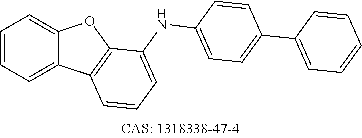 | 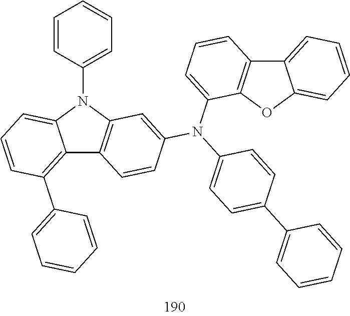 [SMILES: c1ccc(-c2ccc(N(c3ccc4c5c(-c6ccccc6)cccc5n(-c5ccccc5)c4c3)c3cccc4c3oc3ccccc34)cc2)cc1] | 72 |
| | 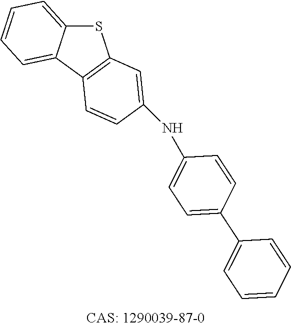 | 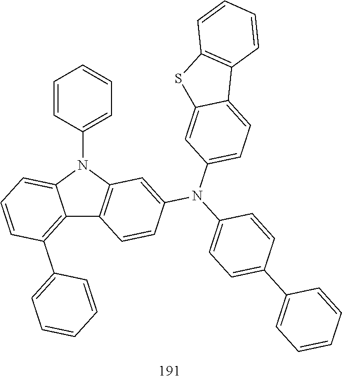 [SMILES: c1ccc(-c2ccc(N(c3ccc4c(c3)sc3ccccc34)c3ccc4c5c(-c6ccccc6)cccc5n(-c5ccccc5)c4c3)cc2)cc1] | 70 |
| | 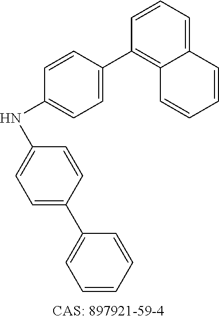 | 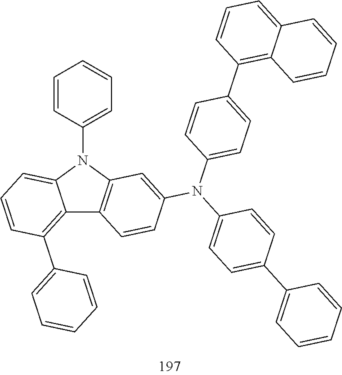 [SMILES: c1ccc(-c2ccc(N(c3ccc(-c4cccc5ccccc45)cc3)c3ccc4c5c(-c6ccccc6)cccc5n(-c5ccccc5)c4c3)cc2)cc1] | 71 |
| | 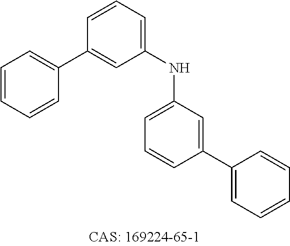 | 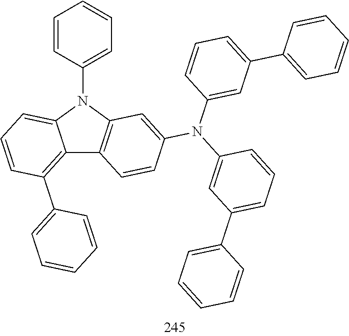 [SMILES: c1ccc(-c2cccc(N(c3cccc(-c4ccccc4)c3)c3ccc4c5c(-c6ccccc6)cccc5n(-c5ccccc5)c4c3)c2)cc1] | 70 |
| 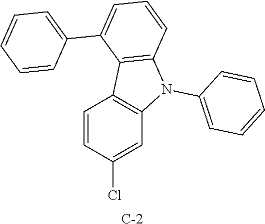 | 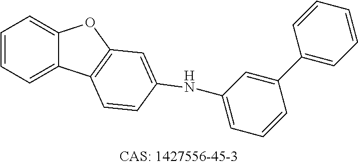 | 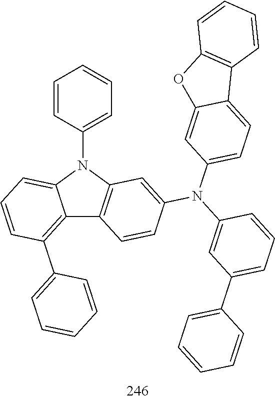 [SMILES: c1ccc(-c2cccc(N(c3ccc4c(c3)oc3ccccc34)c3ccc4c5c(-c6ccccc6)cccc5n(-c5ccccc5)c4c3)c2)cc1] | 71 |
| | 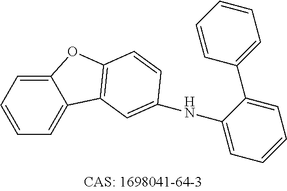 | 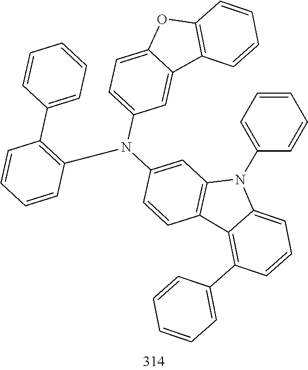 [SMILES: c1ccc(-c2ccccc2N(c2ccc3oc4ccccc4c3c2)c2ccc3c4c(-c5ccccc5)cccc4n(-c4ccccc4)c3c2)cc1] | 72 |
| 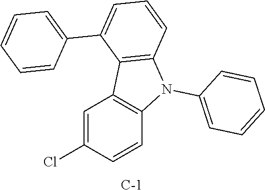 | 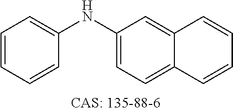 | 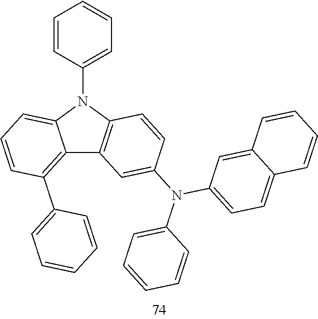 [SMILES: c1ccc(-c2cccc3c2c2cc(N(c4ccccc4)c4ccc5ccccc5c4)ccc2n3-c2ccccc2)cc1] | 70 |
| | 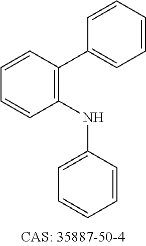 | 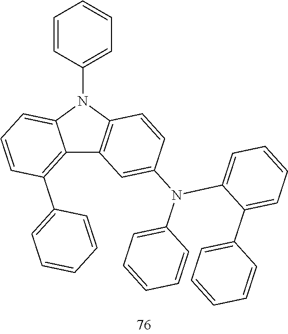 [SMILES: c1ccc(-c2ccccc2N(c2ccccc2)c2ccc3c(c2)c2c(-c4ccccc4)cccc2n3-c2ccccc2)cc1] | 70 |
| | 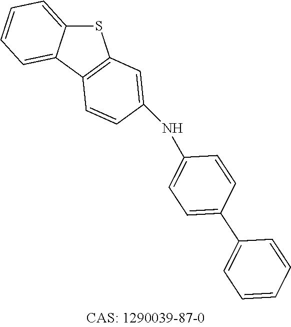 | 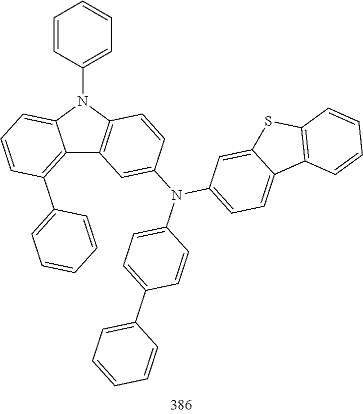 [SMILES: c1ccc(-c2ccc(N(c3ccc4c(c3)sc3ccccc34)c3ccc4c(c3)c3c(-c5ccccc5)cccc3n4-c3ccccc3)cc2)cc1] | 71 |
| | 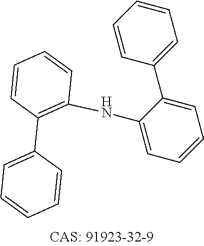 | 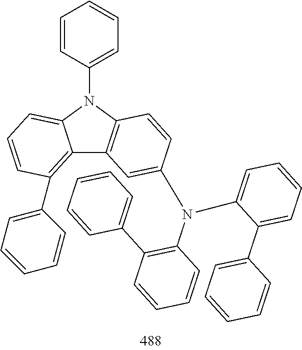 [SMILES: c1ccc(-c2ccccc2N(c2ccc3c(c2)c2c(-c4ccccc4)cccc2n3-c2ccccc2)c2ccccc2-c2ccccc2)cc1] | 71 |
| | 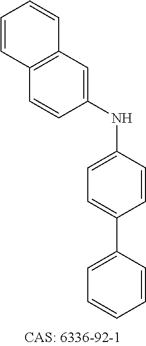 | 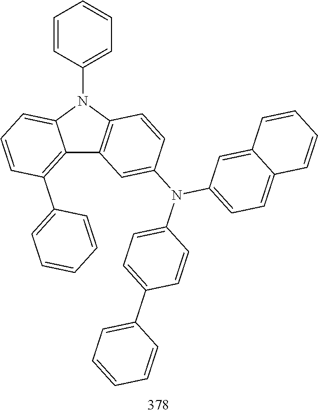 [SMILES: c1ccc(-c2ccc(N(c3ccc4ccccc4c3)c3ccc4c(c3)c3c(-c5ccccc5)cccc3n4-c3ccccc3)cc2)cc1] | 73 |
| | 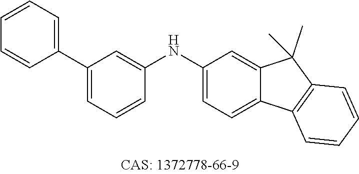 | 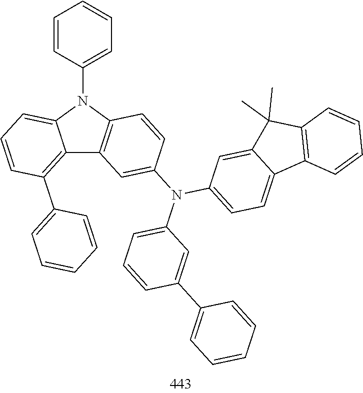 [SMILES: CC1(C)c2ccccc2-c2ccc(N(c3cccc(-c4ccccc4)c3)c3ccc4c(c3)c3c(-c5ccccc5)cccc3n4-c3ccccc3)cc21] | 71 |
| | 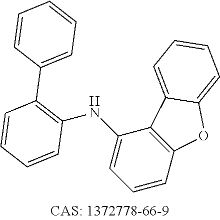 | 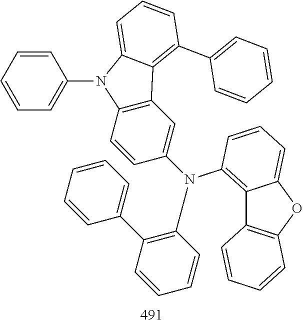 [SMILES: c1ccc(-c2ccccc2N(c2ccc3c(c2)c2c(-c4ccccc4)cccc2n3-c2ccccc2)c2cccc3oc4ccccc4c23)cc1] | 70 |
| 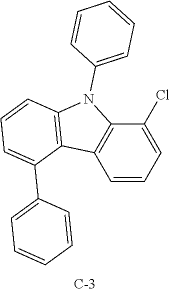 | 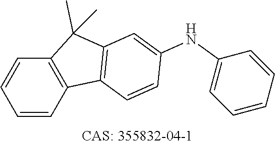 | 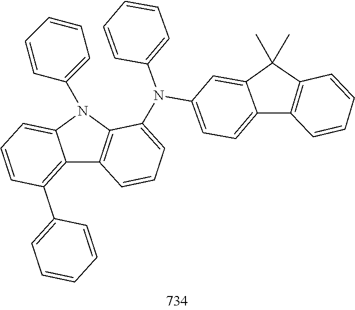 [SMILES: CC1(C)c2ccccc2-c2ccc(N(c3ccccc3)c3cccc4c5c(-c6ccccc6)cccc5n(-c5ccccc5)c34)cc21] | 71 |
| 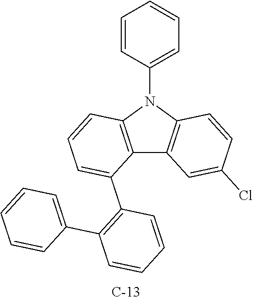 | 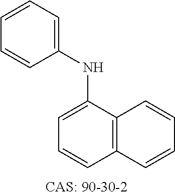 | 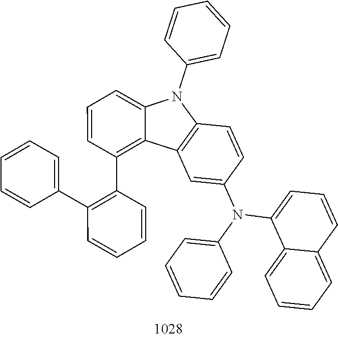 [SMILES: c1ccc(-c2ccccc2-c2cccc3c2c2cc(N(c4ccccc4)c4cccc5ccccc45)ccc2n3-c2ccccc2)cc1] | 70 |
| 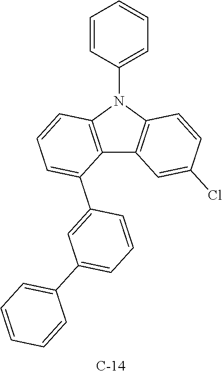 | 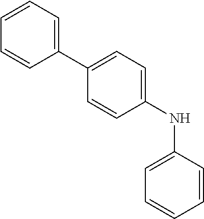 | 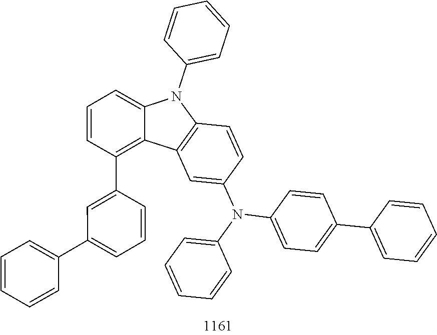 [SMILES: c1ccc(-c2ccc(N(c3ccccc3)c3ccc4c(c3)c3c(-c5cccc(-c6ccccc6)c5)cccc3n4-c3ccccc3)cc2)cc1] | 71 |
| 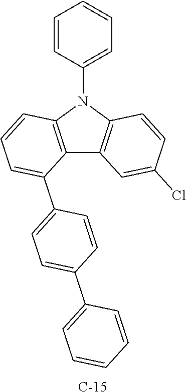 | 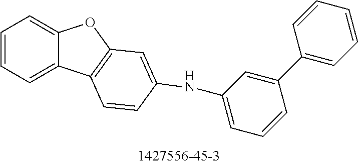 | 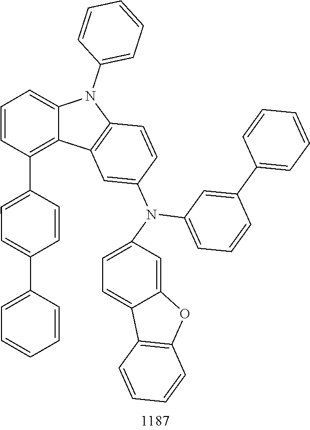 [SMILES: c1ccc(-c2ccc(-c3cccc4c3c3cc(N(c5cccc(-c6ccccc6)c5)c5ccc6c(c5)oc5ccccc56)ccc3n4-c3ccccc3)cc2)cc1] | 70 |
| 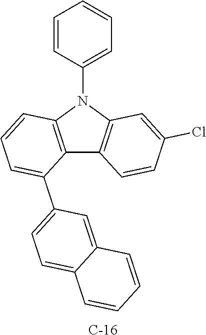 | 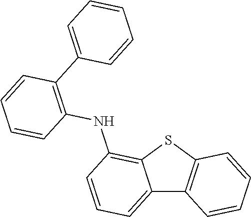 | 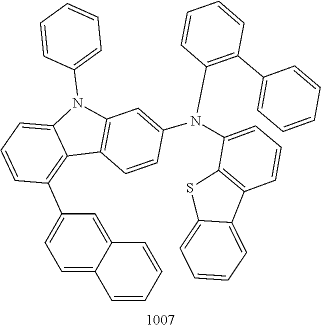 [SMILES: c1ccc(-c2ccccc2N(c2ccc3c4c(-c5ccc6ccccc6c5)cccc4n(-c4ccccc4)c3c2)c2cccc3c2sc2ccccc23)cc1] | 72 |
| 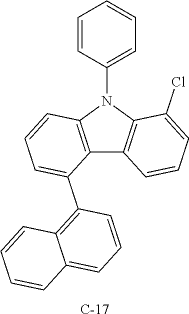 | 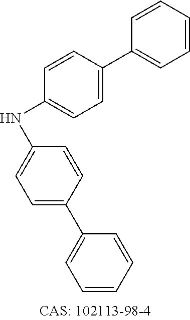 | 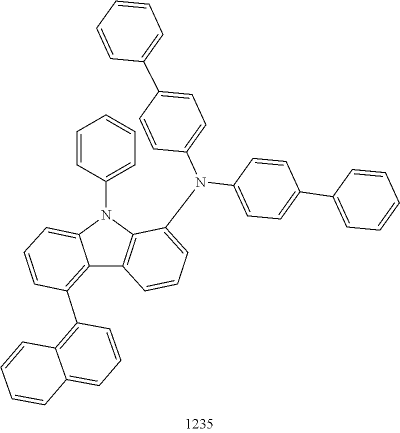 [SMILES: c1ccc(-c2ccc(N(c3ccc(-c4ccccc4)cc3)c3cccc4c5c(-c6cccc7ccccc67)cccc5n(-c5ccccc5)c34)cc2)cc1] | 73 |
| 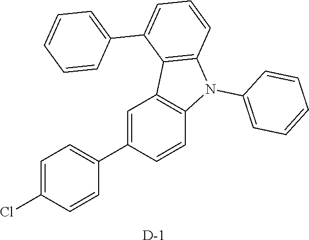 | 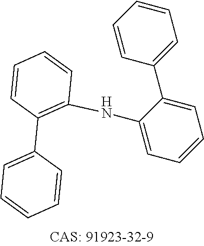 | 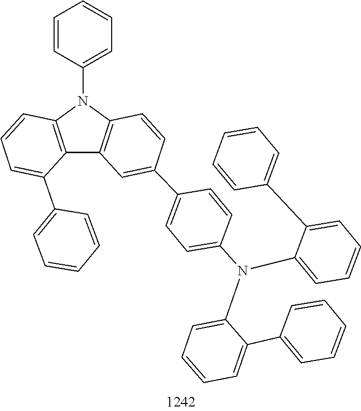 [SMILES: c1ccc(-c2ccccc2N(c2ccc(-c3ccc4c(c3)c3c(-c5ccccc5)cccc3n4-c3ccccc3)cc2)c2ccccc2-c2ccccc2)cc1] | 71 |
| 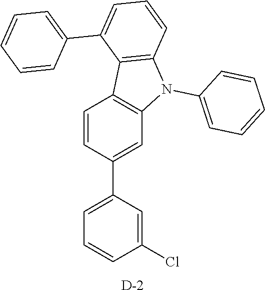 | 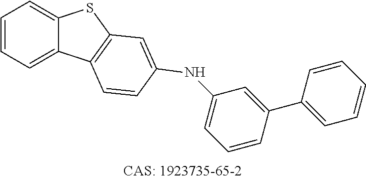 | 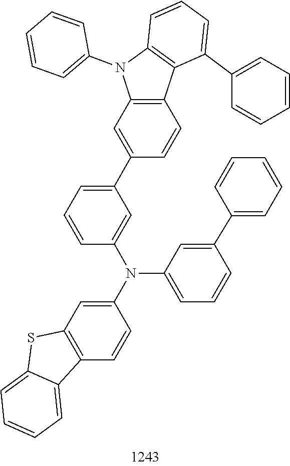 [SMILES: c1ccc(-c2cccc(N(c3cccc(-c4ccc5c6c(-c7ccccc7)cccc6n(-c6ccccc6)c5c4)c3)c3ccc4c(c3)sc3ccccc34)c2)cc1] | 70 |
| 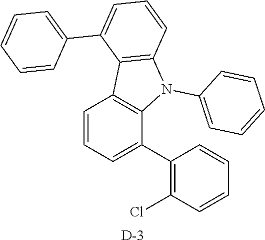 | 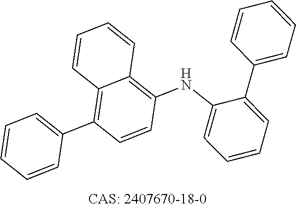 | 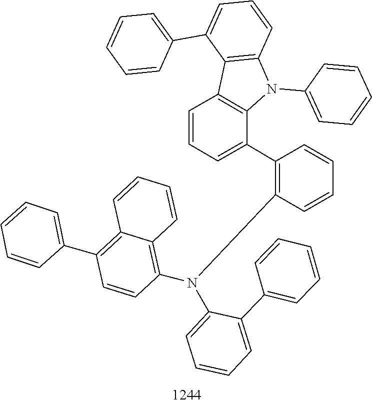 [SMILES: c1ccc(-c2ccccc2N(c2ccccc2-c2cccc3c4c(-c5ccccc5)cccc4n(-c4ccccc4)c23)c2ccc(-c3ccccc3)c3ccccc23)cc1] | 72 |
| 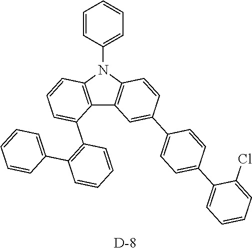 | 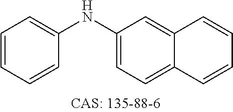 | 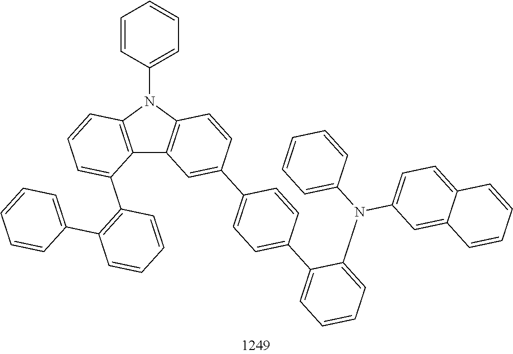 [SMILES: c1ccc(-c2ccccc2-c2cccc3c2c2cc(-c4ccc(-c5ccccc5N(c5ccccc5)c5ccc6ccccc6c5)cc4)ccc2n3-c2ccccc2)cc1] | 72 |
| 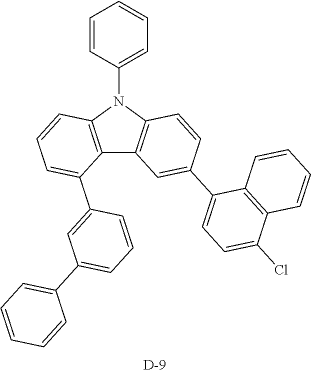 | 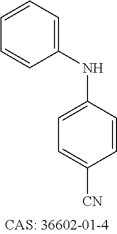 | 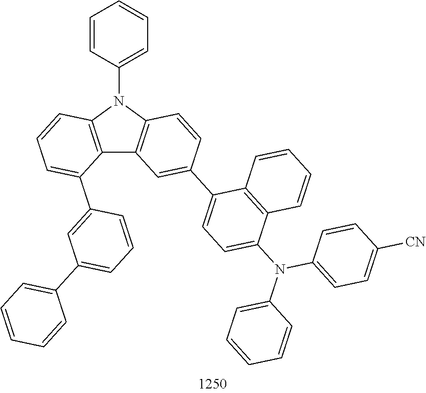 [SMILES: N#Cc1ccc(N(c2ccccc2)c2ccc(-c3ccc4c(c3)c3c(-c5cccc(-c6ccccc6)c5)cccc3n4-c3ccccc3)c3ccccc23)cc1] | 70 |
| 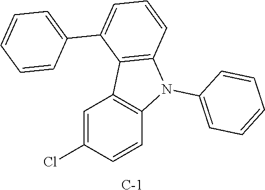 | 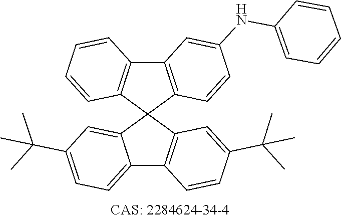 | 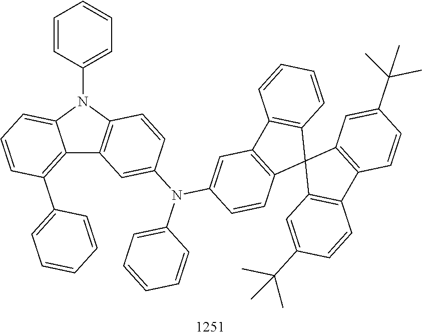 [SMILES: CC(C)(C)c1ccc2c(c1)C1(c3ccccc3-c3cc(N(c4ccccc4)c4ccc5c(c4)c4c(-c6ccccc6)cccc4n5-c4ccccc4)ccc31)c1cc(C(C)(C)C)ccc1-2] | 70 |
| 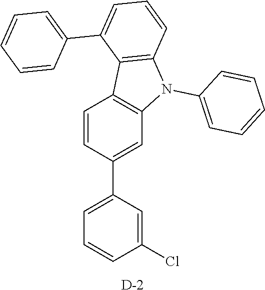 | 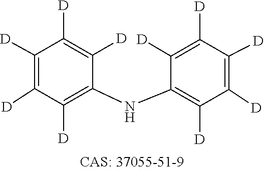 | 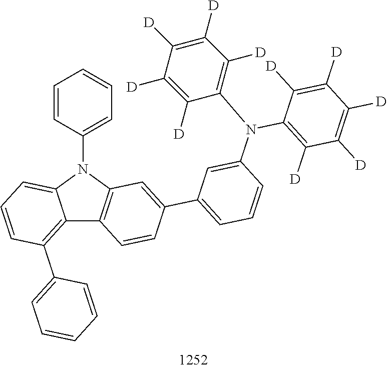 [SMILES: [2H]c1c([2H])c([2H])c(N(c2cccc(-c3ccc4c5c(-c6ccccc6)cccc5n(-c5ccccc5)c4c3)c2)c2c([2H])c([2H])c([2H])c([2H])c2[2H])c([2H])c1[2H]] | 69 |
| 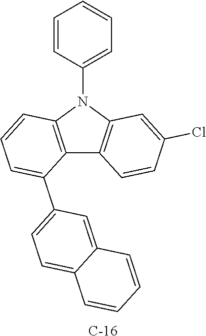 | 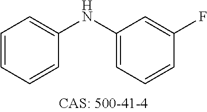 | 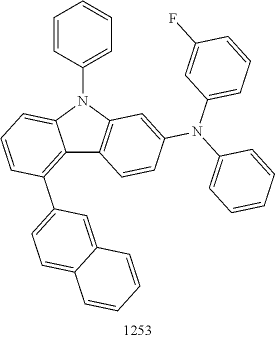 [SMILES: Fc1cccc(N(c2ccccc2)c2ccc3c4c(-c5ccc6ccccc6c5)cccc4n(-c4ccccc4)c3c2)c1] | 70 |
| 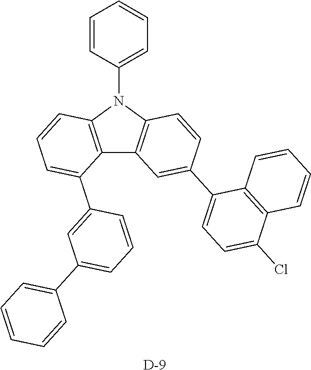 | 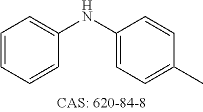 | 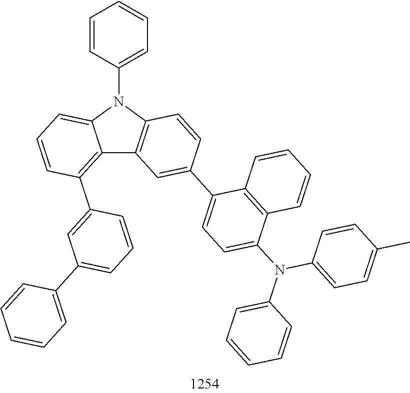 [SMILES: Cc1ccc(N(c2ccccc2)c2ccc(-c3ccc4c(c3)c3c(-c5cccc(-c6ccccc6)c5)cccc3n4-c3ccccc3)c3ccccc23)cc1] | 71 |
| 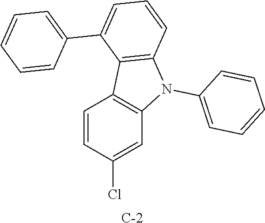 | 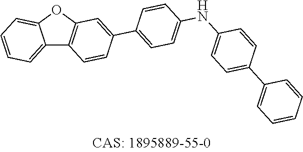 | 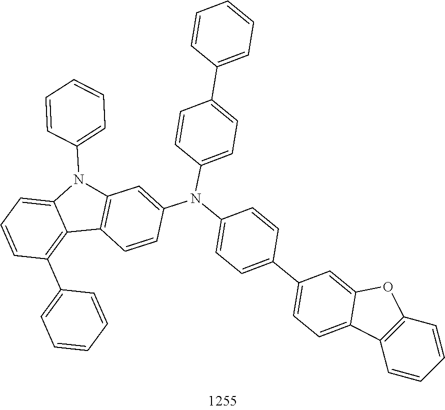 [SMILES: c1ccc(-c2ccc(N(c3ccc(-c4ccc5c(c4)oc4ccccc45)cc3)c3ccc4c5c(-c6ccccc6)cccc5n(-c5ccccc5)c4c3)cc2)cc1] | 70 |
| | 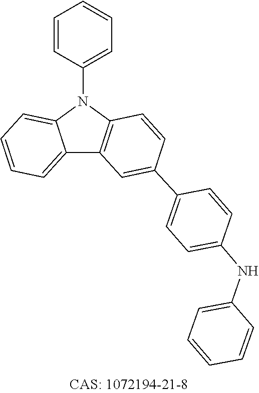 | 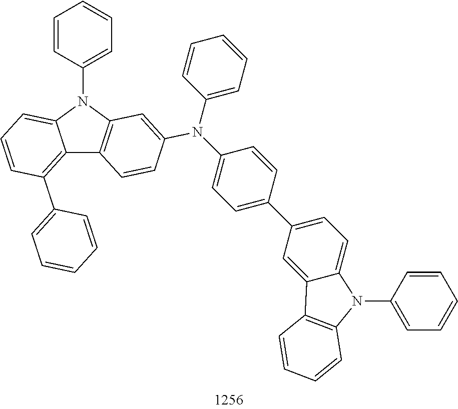 [SMILES: c1ccc(-c2cccc3c2c2ccc(N(c4ccccc4)c4ccc(-c5ccc6c(c5)c5ccccc5n6-c5ccccc5)cc4)cc2n3-c2ccccc2)cc1] | 71 |
| | 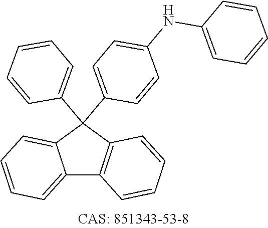 | 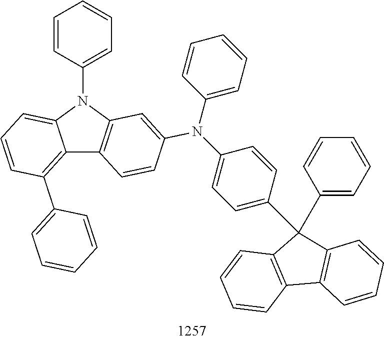 [SMILES: c1ccc(-c2cccc3c2c2ccc(N(c4ccccc4)c4ccc(C5(c6ccccc6)c6ccccc6-c6ccccc65)cc4)cc2n3-c2ccccc2)cc1] | 72 |
| | 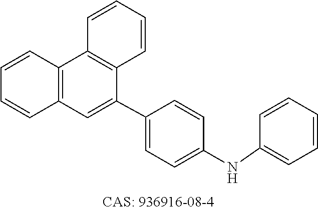 | 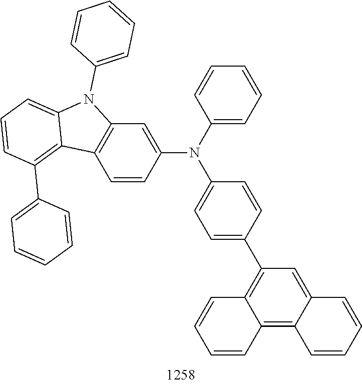 [SMILES: c1ccc(-c2cccc3c2c2ccc(N(c4ccccc4)c4ccc(-c5cc6ccccc6c6ccccc56)cc4)cc2n3-c2ccccc2)cc1] | 71 |
| 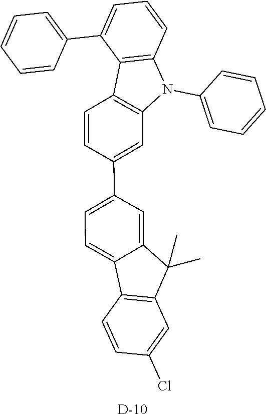 | 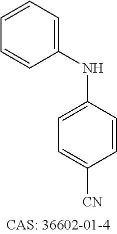 | 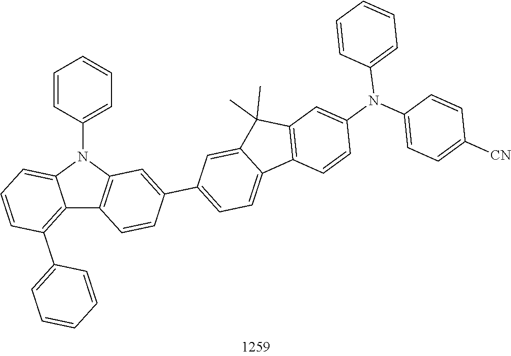 [SMILES: CC1(C)c2cc(-c3ccc4c5c(-c6ccccc6)cccc5n(-c5ccccc5)c4c3)ccc2-c2ccc(N(c3ccccc3)c3ccc(C#N)cc3)cc21] | 70 |
| 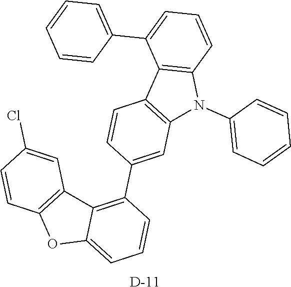 | 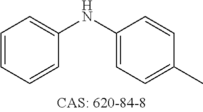 | 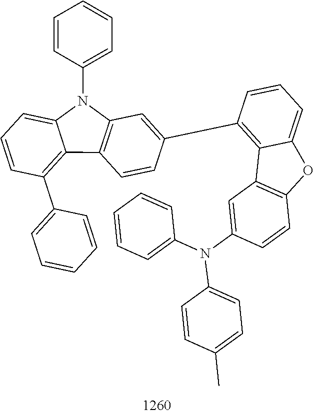 [SMILES: Cc1ccc(N(c2ccccc2)c2ccc3oc4cccc(-c5ccc6c7c(-c8ccccc8)cccc7n(-c7ccccc7)c6c5)c4c3c2)cc1] | 69 |

Compound Characterization

Mass spectrometry analysis was performed on the above synthesized compounds, and the obtained data are shown in Table 9 below.

TABLE 9

| | Mass | | Mass | | Mass |
|---|---|---|---|---|---|
| Compound | spectrum | Compound | spectrum | Compound | spectrum |
| No. | [M + H]⁺ | No. | [M + H]⁺ | No. | [M + H]⁺ |
| 1 | 487.3 | 2 | 537.3 | 4 | 563.3 |
| 7 | 577.3 | 11 | 593.3 | 74 | 537.3 |
| 76 | 563.3 | 186 | 639.3 | 190 | 653.3 |
| 191 | 669.3 | 197 | 689.3 | 245 | 639.3 |
| 246 | 653.3 | 314 | 653.3 | 378 | 613.3 |
| 386 | 669.3 | 443 | 679.3 | 488 | 639.3 |
| 491 | 653.3 | 734 | 603.3 | 1007 | 719.3 |
| 1028 | 613.3 | 1161 | 639.3 | 1187 | 729.3 |
| 1235 | 689.3 | 1242 | 715.3 | 1243 | 745.3 |
| 1244 | 765.3 | 1249 | 765.3 | 1250 | 714.3 |
| 1251 | 837.4 | 1252 | 573.3 | 1253 | 555.2 |
| 1254 | 703.3 | 1255 | 729.3 | 1256 | 728.3 |
| 1257 | 727.3 | 1258 | 663.3 | 1259 | 704.3 |
| 1260 | 667.3 | | | | |

NMR data for some compounds and intermediates are shown in Table 10 below:

TABLE 10

| Intermediate/ | |
|---|---|
| Compound | NMR data |
| Intermediate IM- | ¹H NMR (400 Hz, CD₂Cl₂): 8.34 (s, 1H), 7.61 (d, 1H), 7.56-7.52 (m, |
| B-1 | 2H), 7.50-7.42 (m, 5H), 7.32-7.36 (m, 1H), 7.10 (d, 1H), 6.98-6.94 (m, |
| | 1H). |
| Intermediate IM- | ¹H NMR (400 Hz, CD₂Cl₂): 7.66-7.63 (m, 4H), 7.58-7.53 (m, 6H), 7.48- |
| C-1 | 7.45 (m, 1H), 7.43 (s, 1H), 7.36 (d, 1H), 7.28 (d, 2H), 7.16 (d, 1H). |
| Compound 186 | ¹H NMR (400 Hz, CD₂Cl₂): 7.76 (d, 1H), 7.70-7.66 (m, 2H), 7.53-7.41 |
| | (m, 21H), 7.35-7.26 (m, 3H), 7.14 (d, 1H), 7.11 (d, 4H), 7.08 (s, 1H), |
| | 6.95 (d, 1H). |
| Compound 197 | ¹H NMR (400 Hz, CD₂Cl₂): 7.87 (d, 1H), 7.84 (d, 1H), 7.73-7.68 (m, |
| | 2H), 7.65-7.57 (m, 2H), 7.51-7.30 (m, 18H), 7.26-7.22 (m, 5H), 7.14 |
| | (d, 1H), 7.09 (d, 2H), 7.05 (d, 2H), 7.01 (s, 1H), 6.97 (d, 1H). |
| Compound 491 | ¹H NMR (400 Hz, CD₂Cl₂): 7.67 (d, 1H), 7.61-7.49 (m, 2H), 7.38-7.16 |
| | (m, 26H), 6.83 (d, 2H), 6.68 (d, 1H). |

Device Examples

### Example 1: Green Organic Electroluminescent Device

An anode was prepared by the following process: a substrate (manufactured by Corning) having an ITO thickness of 1500 Å was cut into a size of 40 mm×40 mm×0.7 mm to be prepared into an experimental substrate with a cathode, an anode and an insulating layer pattern by a photoetching process, and surface treatment was performed by ultraviolet ozone and O₂:N₂ plasma to increase the work function of the anode (the experimental substrate) and clean scum.

F4-TCNQ was vacuum evaporated on the experimental substrate (the anode) to form a hole injection layer (HIL) with a thickness of 100 Å, and NPB was evaporated on the hole injection layer to form a hole transport layer with a thickness of 980 Å.

A compound 1 was vacuum evaporated on the hole transport layer to form an electron blocking layer with a thickness of 400 Å.

GH-n1, GH-n2 and Ir(ppy)₃ were co-evaporated at a ratio of 50%:45%:5% (an evaporation rate) on the electron blocking layer to form a green organic light-emitting layer (EML) with a thickness of 400 Å.

ET-06 and LiQ were mixed at a weight ratio of 1:1 and evaporated to form an electron transport layer (ETL) with a thickness of 300 Å, LiQ was evaporated on the electron transport layer to form an electron injection layer (EIL) with a thickness of 10 Å, and then magnesium (Mg) and silver (Ag) were mixed and vacuum evaporated at an evaporation rate of 1:9 on the electron injection layer to form a cathode with a thickness of 105 Å.

CP-05 with a thickness of 650 Å was evaporated on the cathode to form an organic capping layer (CPL), thus completing the manufacture of an organic light-emitting device.

### Examples 2 to 40

An organic electroluminescent device was manufactured by the same method as that in Example 1 by using compounds shown in Table 12 instead of the compound 1 in Example 1 when the electron blocking layer was formed.

### Comparative Example 1

An organic electroluminescent device was manufactured by the same method as that in Example 1 by using a compound A shown in Table 11 instead of the compound 1 in Example 1 when the electron blocking layer was formed.

### Comparative Example 2

An organic electroluminescent device was manufactured by the same method as that in Example 1 by using a compound B shown in Table 11 instead of the compound 1 in Example 1 when the electron blocking layer was formed.

### Comparative Example 3

An organic electroluminescent device was manufactured by the same method as that in Example 1 by using a compound C shown in Table 11 instead of the compound 1 in Example 1 when the electron blocking layer was formed.

### Comparative Example 4

An organic electroluminescent device was manufactured by the same method as that in Example 1 by using a compound D shown in Table 11 instead of the compound 1 in Example 1 when the electron blocking layer was formed.

Structural formulas of materials used in Examples 1 to 40 and Comparative Examples 1 to 4 are shown in Table 11 below:

TABLE 11

| | |
|---|---|
| 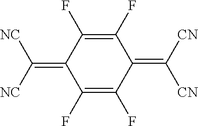 [SMILES: N#CC(C#N)=c1c(F)c(F)c(=C(C#N)C#N)c(F)c1F] | F4-TCNQ |
| 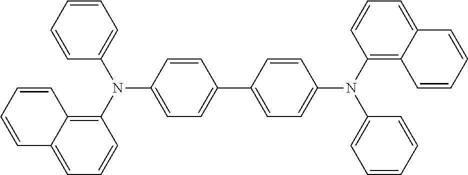 [SMILES: c1ccc(N(c2ccc(-c3ccc(N(c4ccccc4)c4cccc5ccccc45)cc3)cc2)c2cccc3ccccc23)cc1] | NPB |
| 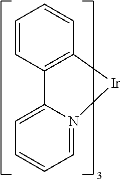 | Ir(ppy)₃ |
| 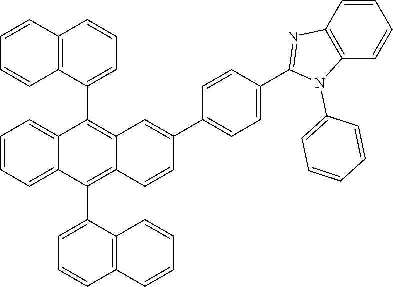 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1] | ET-06 |
| 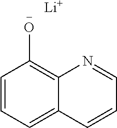 [SMILES: [Li+] ; Oc1cccc2cccnc12] | LiQ |
| 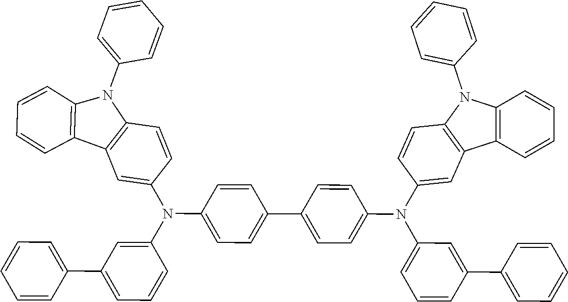 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1] | CP-05 |
| 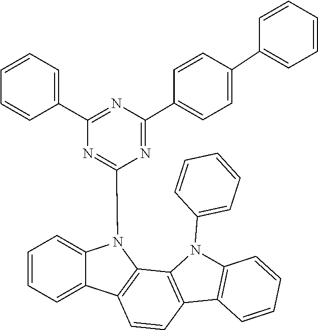 [SMILES: c1ccc(-c2ccc(-c3nc(-c4ccccc4)nc(-n4c5ccccc5c5ccc6c7ccccc7n(-c7ccccc7)c6c54)n3)cc2)cc1] | GH-n1 |
| 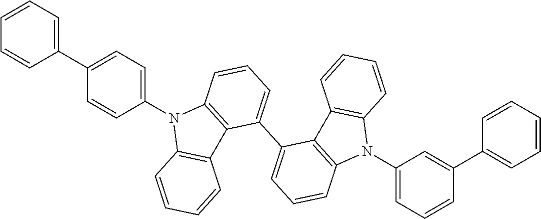 [SMILES: c1ccc(-c2ccc(-n3c4ccccc4c4c(-c5cccc6c5c5ccccc5n6-c5cccc(-c6ccccc6)c5)cccc43)cc2)cc1] | GH-n2 |
| 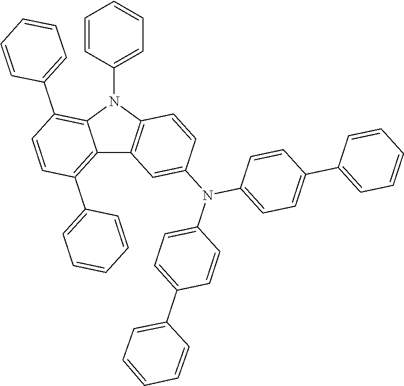 [SMILES: c1ccc(-c2ccc(N(c3ccc(-c4ccccc4)cc3)c3ccc4c(c3)c3c(-c5ccccc5)ccc(-c5ccccc5)c3n4-c3ccccc3)cc2)cc1] | Compound A |
| 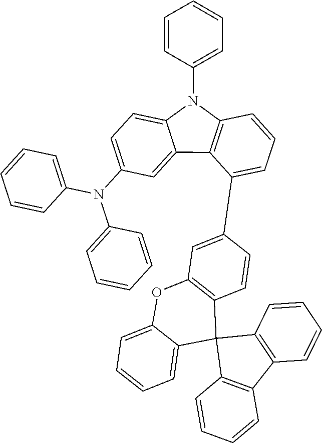 [SMILES: c1ccc(N(c2ccccc2)c2ccc3c(c2)c2c(-c4ccc5c(c4)Oc4ccccc4C54c5ccccc5-c5ccccc54)cccc2n3-c2ccccc2)cc1] | Compound B |
| 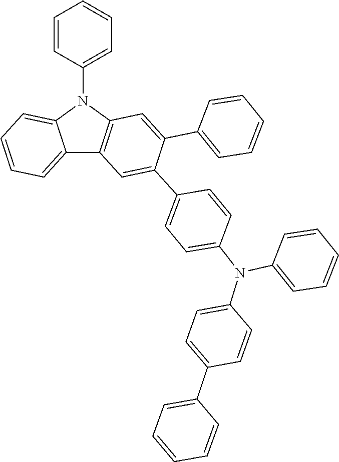 [SMILES: c1ccc(-c2ccc(N(c3ccccc3)c3ccc(-c4cc5c6ccccc6n(-c6ccccc6)c5cc4-c4ccccc4)cc3)cc2)cc1] | Compound C |
| 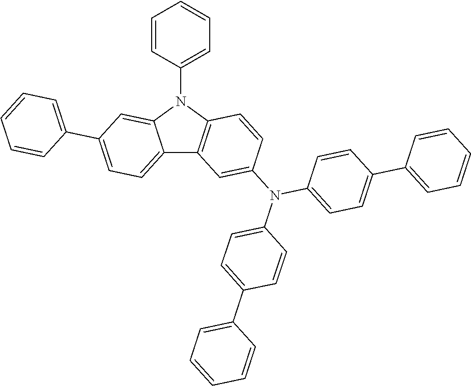 [SMILES: c1ccc(-c2ccc(N(c3ccc(-c4ccccc4)cc3)c3ccc4c(c3)c3ccc(-c5ccccc5)cc3n4-c3ccccc3)cc2)cc1] | Compound D |

For the manufactured organic electroluminescent devices, the performance of the devices was analyzed under a condition of 20 mA/cm², and the results are shown in the Table 12 below:

TABLE 12

| | | | | | | | External | T95 |
|---|---|---|---|---|---|---|---|---|
| | | | | | | | quantum | service |
| | | Driving | Current | Power | Chromaticity | Chromaticity | efficiency | life |
| | Compound | voltage | efficiency | efficiency | coordinate | coordinate | EQE | (h)@20 |
| No. | No. | (V) | (Cd/A) | (lm/W) | CIEx | CIEy | (%) | mA/cm² |
| Example 1 | 1 | 3.83 | 81.45 | 66.81 | 0.22 | 0.73 | 20.36 | 283 |
| Example 2 | 2 | 3.85 | 86.47 | 70.56 | 0.22 | 0.73 | 21.62 | 312 |
| Example 3 | 4 | 3.94 | 86.31 | 68.82 | 0.22 | 0.73 | 21.58 | 291 |
| Example 4 | 7 | 3.80 | 83.10 | 68.70 | 0.22 | 0.73 | 20.78 | 286 |
| Example 5 | 11 | 3.81 | 86.54 | 71.36 | 0.22 | 0.73 | 21.64 | 317 |
| Example 6 | 186 | 3.78 | 86.36 | 71.77 | 0.22 | 0.73 | 21.59 | 297 |
| Example 7 | 190 | 3.77 | 84.25 | 70.20 | 0.22 | 0.73 | 21.06 | 299 |
| Example 8 | 191 | 3.84 | 82.24 | 67.28 | 0.22 | 0.73 | 20.56 | 299 |
| Example 9 | 197 | 3.73 | 85.18 | 71.74 | 0.22 | 0.73 | 21.30 | 316 |
| Example 10 | 245 | 3.85 | 82.51 | 67.33 | 0.22 | 0.73 | 20.63 | 295 |
| Example 11 | 246 | 3.75 | 82.19 | 68.85 | 0.22 | 0.73 | 20.55 | 294 |
| Example 12 | 314 | 3.93 | 82.83 | 66.21 | 0.22 | 0.73 | 20.71 | 313 |
| Example 13 | 74 | 3.76 | 80.80 | 67.51 | 0.22 | 0.73 | 20.20 | 284 |
| Example 14 | 76 | 3.86 | 82.72 | 67.32 | 0.22 | 0.73 | 20.68 | 283 |
| Example 15 | 488 | 3.93 | 80.27 | 64.16 | 0.22 | 0.73 | 20.07 | 303 |
| Example 16 | 378 | 3.81 | 85.40 | 70.42 | 0.22 | 0.73 | 21.35 | 291 |
| Example 17 | 443 | 3.84 | 85.52 | 69.96 | 0.22 | 0.73 | 21.38 | 303 |
| Example 18 | 491 | 3.80 | 82.31 | 68.05 | 0.22 | 0.73 | 20.58 | 293 |
| Example 19 | 386 | 3.91 | 82.31 | 66.13 | 0.22 | 0.73 | 20.58 | 309 |
| Example 20 | 734 | 3.77 | 86.06 | 71.71 | 0.22 | 0.73 | 21.52 | 311 |
| Example 21 | 1028 | 3.79 | 87.38 | 72.43 | 0.22 | 0.73 | 21.85 | 311 |
| Example 22 | 1161 | 3.75 | 85.84 | 71.91 | 0.22 | 0.73 | 21.46 | 309 |
| Example 23 | 1187 | 3.75 | 81.65 | 68.40 | 0.22 | 0.73 | 20.41 | 311 |
| Example 24 | 1007 | 3.78 | 81.74 | 67.93 | 0.22 | 0.73 | 20.44 | 292 |
| Example 25 | 1235 | 3.89 | 86.50 | 69.86 | 0.22 | 0.73 | 21.63 | 307 |
| Example 26 | 1242 | 3.72 | 83.21 | 70.27 | 0.22 | 0.73 | 20.80 | 310 |
| Example 27 | 1243 | 3.87 | 84.89 | 68.91 | 0.22 | 0.73 | 21.22 | 293 |
| Example 28 | 1244 | 3.85 | 83.30 | 67.97 | 0.22 | 0.73 | 20.83 | 309 |
| Example 29 | 1249 | 3.93 | 82.09 | 65.62 | 0.22 | 0.73 | 20.52 | 303 |
| Example 30 | 1250 | 3.76 | 82.54 | 68.96 | 0.22 | 0.73 | 20.64 | 285 |
| Example 31 | 1251 | 3.93 | 82.32 | 65.80 | 0.22 | 0.73 | 20.58 | 309 |
| Example 32 | 1252 | 3.88 | 83.70 | 67.77 | 0.22 | 0.73 | 20.93 | 292 |
| Example 33 | 1253 | 3.79 | 80.54 | 66.76 | 0.22 | 0.73 | 20.14 | 290 |
| Example 34 | 1254 | 3.73 | 85.59 | 72.09 | 0.22 | 0.73 | 21.40 | 289 |
| Example 35 | 1255 | 3.80 | 80.93 | 66.91 | 0.22 | 0.73 | 20.23 | 296 |
| Example 36 | 1256 | 3.91 | 81.99 | 65.88 | 0.22 | 0.73 | 20.50 | 314 |
| Example 37 | 1257 | 3.78 | 80.21 | 66.66 | 0.22 | 0.73 | 20.05 | 309 |
| Example 38 | 1258 | 3.72 | 87.03 | 73.50 | 0.22 | 0.73 | 21.76 | 319 |
| Example 39 | 1259 | 3.79 | 81.05 | 67.18 | 0.22 | 0.73 | 20.26 | 295 |
| Example 40 | 1260 | 3.89 | 83.93 | 67.78 | 0.22 | 0.73 | 20.98 | 294 |
| Comparative | Compound | 4.12 | 64.55 | 49.82 | 0.22 | 0.73 | 16.14 | 256 |
| Example 1 | A | | | | | | | |
| Comparative | Compound | 4.18 | 61.11 | 47.05 | 0.22 | 0.73 | 15.28 | 247 |
| Example 2 | B | | | | | | | |
| Comparative | Compound | 4.27 | 63.56 | 48.70 | 0.22 | 0.73 | 15.89 | 255 |
| Example 3 | C | | | | | | | |
| Comparative | Compound | 4.15 | 65.39 | 50.72 | 0.22 | 0.73 | 16.35 | 258 |
| Example 4 | D | | | | | | | |

From the results of Table 12, it can be seen that Examples 1 to 40 in which the compounds were used as the electron blocking layer have the advantages that for the organic electroluminescent device manufactured by using the compounds as the electron blocking layer in the present disclosure, the driving voltage was reduced by at least 0.18 V, the current efficiency was improved by at least 22.8%, the external quantum efficiency was improved by at least 22.6%, and the service life was improved by at least 9.69% compared with Comparative Examples 1 to 4 using well-known compounds A, B, C and D.

Preferred embodiments of the present disclosure have been described above in detail with reference to the accompanying drawings, but the present disclosure is not limited to specific details in the above-described examples, and many simple modifications may be made to the technical solutions of the present disclosure within the technical idea of the present disclosure, and these simple modifications all fall within the scope of protection of the present disclosure.

In addition, the various embodiments of the present disclosure can also be combined at will, as long as they do not violate the idea of the present disclosure, they should also be regarded as the contents disclosed in the present disclosure.

1. A nitrogen-containing compound, having a structure represented by a formula F-1: 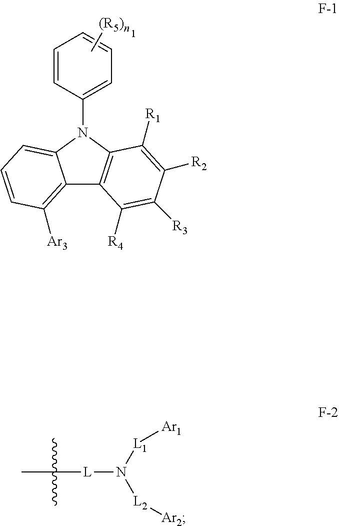
wherein [symbol] represents a chemical bond;
L, L₁ and L₂ are the same as or different from each other, and are respectively and independently selected from a single bond, substituted or unsubstituted arylene with 6 to 30 carbon atoms, and substituted or unsubstituted heteroarylene with 3 to 30 carbon atoms;
Ar₁ and Ar₂ are the same as or different from each other, and are respectively and independently selected from substituted or unsubstituted aryl with 6 to 40 carbon atoms, and substituted or unsubstituted heteroaryl with 3 to 30 carbon atoms;
Ar₃ is selected from substituted or unsubstituted aryl with 6 to 20 carbon atoms;
R₁, R₂, R₃, and R₄ are the same as or different from each other, and are respectively and independently selected from hydrogen or a group represented by a formula F-2, and one of R₁, R₂, R₃, and R₄ is the group represented by the formula F-2;
R₅ is selected from deuterium, cyano, a halogen group, substituted or unsubstituted alkyl with 1 to 10 carbon atoms, substituted or unsubstituted cycloalkyl with 3 to 20 carbon atoms, substituted or unsubstituted aryl with 6 to 30 carbon atoms, and substituted or unsubstituted heteroaryl with 3 to 30 carbon atoms;
n₁ represents the number of R₅, and n₁ is 0, 1, 2, 3, 4, or 5;
substituents in L, L₁, L₂, Ar₁, Ar₃, and R₅ are each independently selected from deuterium, a halogen group, cyano, heteroaryl with 3 to 20 carbon atoms, aryl with 6 to 20 carbon atoms optionally substituted with 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, alkenyl with 2 to 6 carbon atoms, alkynyl with 2 to 6 carbon atoms, cycloalkyl with 3 to 10 carbon atoms, heterocycloalkyl with 2 to 10 carbon atoms, cycloalkenyl with 5 to 10 carbon atoms, heterocycloalkenyl with 4 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms;
substituent in Ar₃ is selected from deuterium, a halogen group, cyano, and phenyl.

2. The nitrogen-containing compound of claim 1, wherein the nitrogen-containing compound is selected from compounds represented by any one of the following chemical formulae:

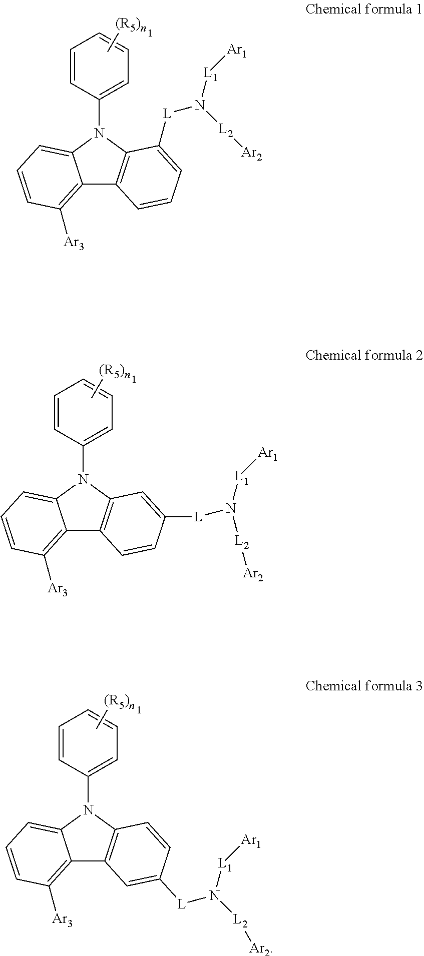

3. The nitrogen-containing compound of claim 1, wherein the L, L₁ and L₂ are the same as or different from each other, and are respectively and independently selected from a single bond, substituted or unsubstituted arylene with 6 to 20 carbon atoms, and substituted or unsubstituted heteroarylene with 12 to 20 carbon atoms.

4. The nitrogen-containing compound of claim 1, wherein the L, L₁ and L₂ are the same as or different from each other, and are respectively and independently selected from a single bond, substituted or unsubstituted phenylene, substituted or unsubstituted naphthylene, substituted or unsubstituted biphenylene, substituted or unsubstituted dimethylfluorenylene, substituted or unsubstituted dibenzofurylene, substituted or unsubstituted carbazolylene, substituted or un substituted dibenzothienylene, and substituted or unsubstituted N-phenylcarbazolylene.

5. The nitrogen-containing compound of claim 1, wherein the L, L₁ and L₂ are each independently selected from a single bond, or a substituted or unsubstituted group W, wherein the unsubstituted group W is selected from a group consisting of the following groups: 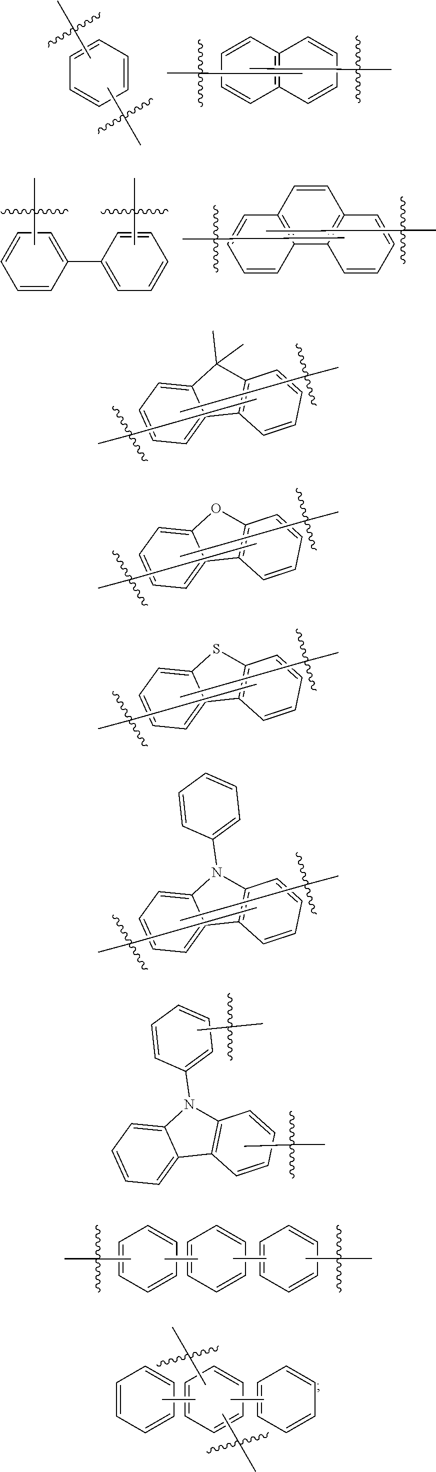
wherein [symbol] represents a chemical bond; the substituted group W has one or more substituents, and the substituents are each independently selected from deuterium, cyano, a halogen group, methyl, ethyl, n-propyl, isopropyl, tert-butyl, phenyl, naphthyl, biphenyl, phenanthryl, and anthryl; when the number of the substituents in the group W is greater than 1, the substituents are the same or different.

6. The nitrogen-containing compound of claim 1, wherein the Ar₁ and Ar₂ are the same as or different from each other, and are respectively and independently selected from substituted or unsubstituted aryl with 6 to 33 carbon atoms, and substituted or unsubstituted heteroaryl with 3 to 25 carbon atoms.

7. The nitrogen-containing compound of claim 1, wherein the Ar₃ is selected from substituted or unsubstituted aryl with 6 to 12 carbon atoms.

8. The nitrogen-containing compound of claim 1, wherein the Ar₁ and Ar₂ are each independently selected from a substituted or unsubstituted group V, wherein the unsubstituted group V is selected from a group consisting of the following groups: 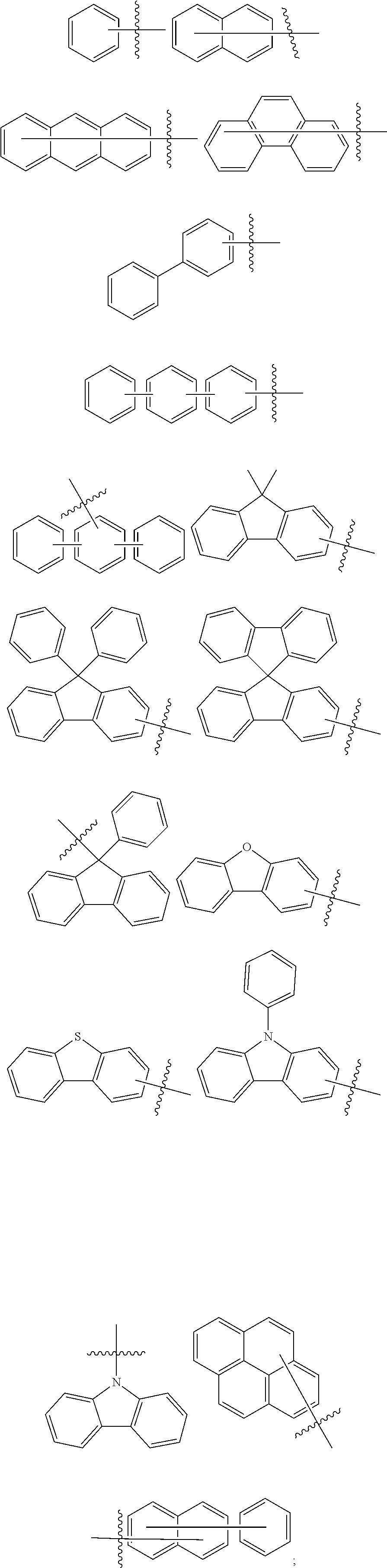
wherein [symbol] represents a chemical bond; the substituted group V has one or more substituents, and the substituents are each independently selected from deuterium, cyano, a halogen group, methyl, ethyl, n-propyl, isopropyl, tert-butyl, phenyl, naphthyl, biphenyl, phenanthryl, anthryl, dibenzofuranyl, dibenzothienyl, carbazolyl, and N-phenylcarbazolyl; when the number of the substituents in the group V is greater than 1, the substituents are the same or different.

9. The nitrogen-containing compound of claim 1, wherein the nitrogen-containing compound is selected from a group consisting of the following compounds:

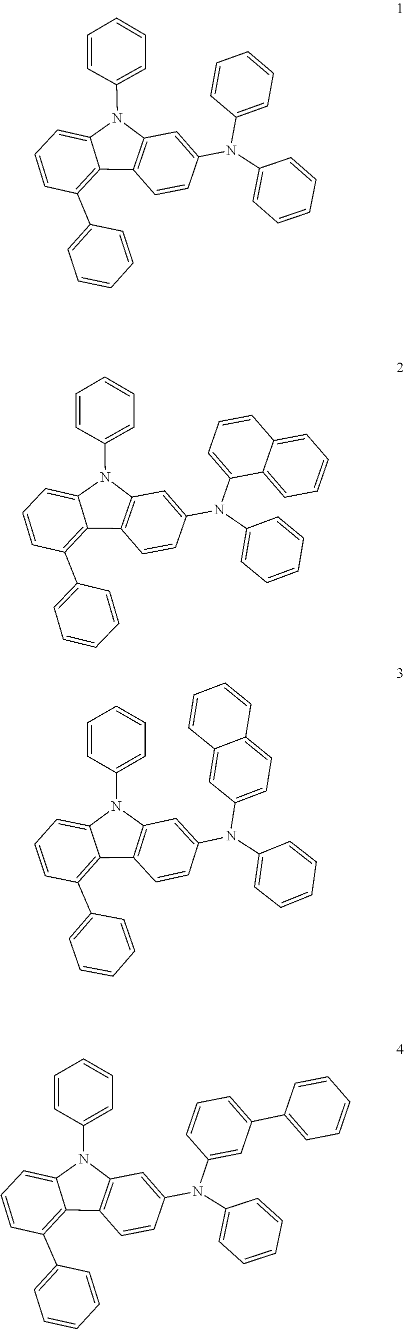 [SMILES: c1ccc(-c2cccc3c2c2ccc(N(c4ccccc4)c4ccccc4)cc2n3-c2ccccc2)cc1 ; c1ccc(-c2cccc3c2c2ccc(N(c4ccccc4)c4cccc5ccccc45)cc2n3-c2ccccc2)cc1 ; c1ccc(-c2cccc3c2c2ccc(N(c4ccccc4)c4ccc5ccccc5c4)cc2n3-c2ccccc2)cc1 ; c1ccc(-c2cccc(N(c3ccccc3)c3ccc4c5c(-c6ccccc6)cccc5n(-c5ccccc5)c4c3)c2)cc1]

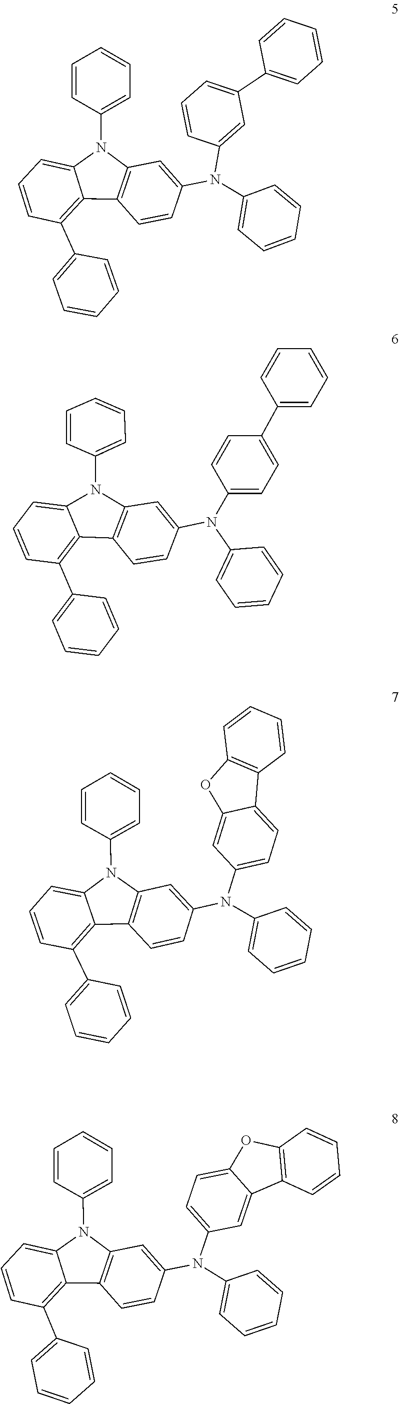

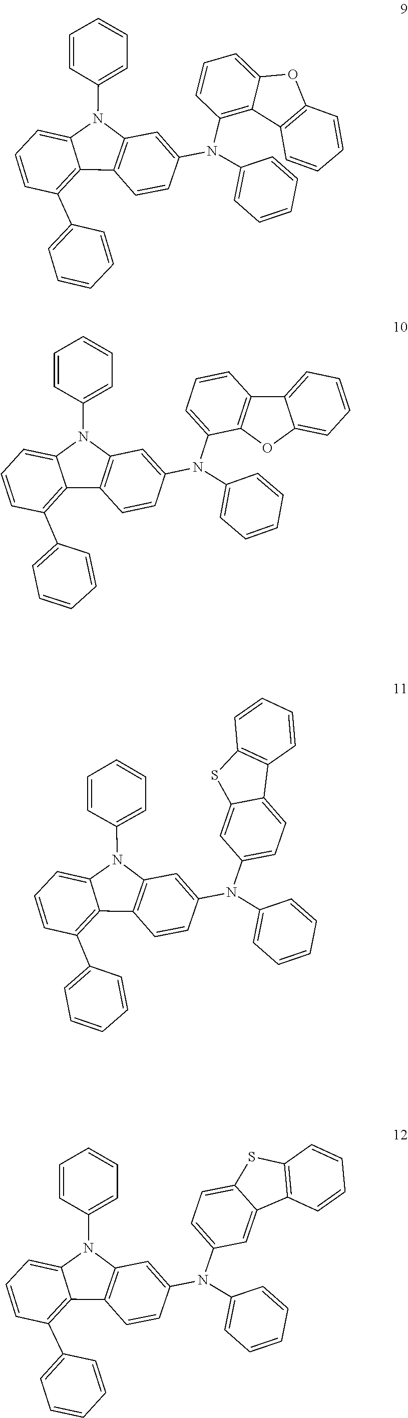 [SMILES: c1ccc(-c2cccc3c2c2ccc(N(c4ccccc4)c4cccc5oc6ccccc6c45)cc2n3-c2ccccc2)cc1 ; c1ccc(-c2cccc3c2c2ccc(N(c4ccccc4)c4cccc5c4oc4ccccc45)cc2n3-c2ccccc2)cc1 ; c1ccc(-c2cccc3c2c2ccc(N(c4ccccc4)c4ccc5c(c4)sc4ccccc45)cc2n3-c2ccccc2)cc1 ; c1ccc(-c2cccc3c2c2ccc(N(c4ccccc4)c4ccc5sc6ccccc6c5c4)cc2n3-c2ccccc2)cc1]

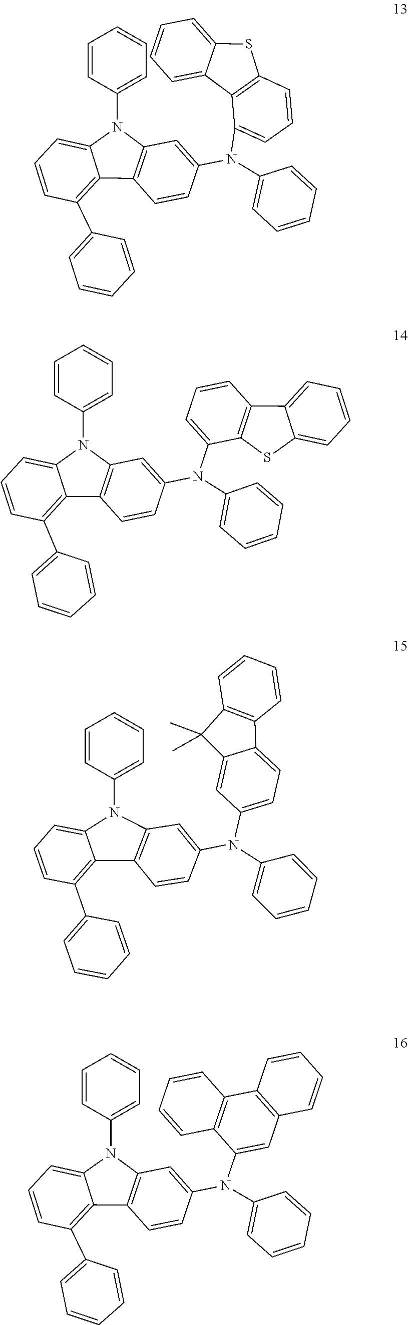 [SMILES: c1ccc(-c2cccc3c2c2ccc(N(c4ccccc4)c4cccc5sc6ccccc6c45)cc2n3-c2ccccc2)cc1 ; c1ccc(-c2cccc3c2c2ccc(N(c4ccccc4)c4cccc5c4sc4ccccc45)cc2n3-c2ccccc2)cc1 ; CC1(C)c2ccccc2-c2ccc(N(c3ccccc3)c3ccc4c5c(-c6ccccc6)cccc5n(-c5ccccc5)c4c3)cc21 ; c1ccc(-c2cccc3c2c2ccc(N(c4ccccc4)c4cc5ccccc5c5ccccc45)cc2n3-c2ccccc2)cc1]

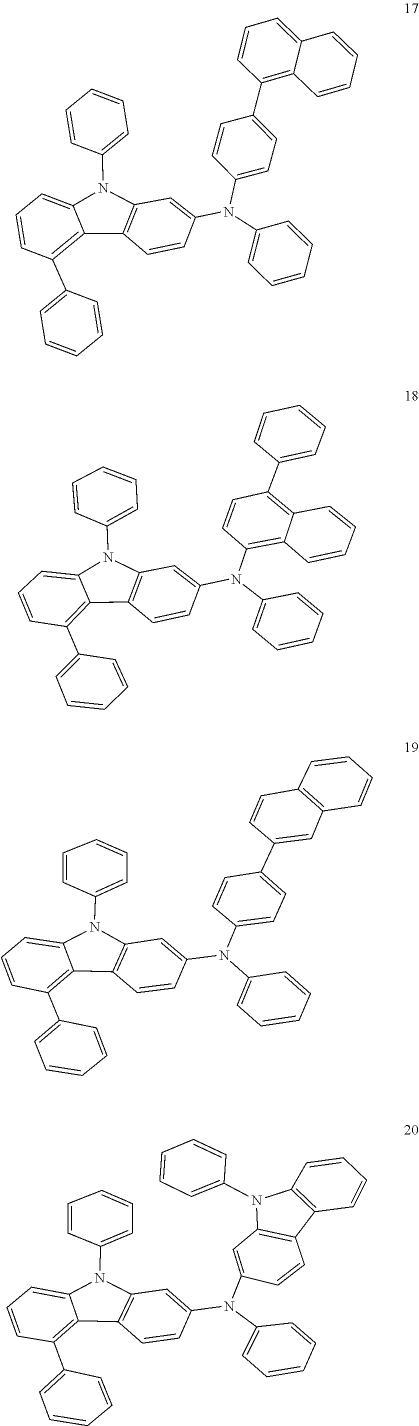 [SMILES: c1ccc(-c2cccc3c2c2ccc(N(c4ccccc4)c4ccc(-c5cccc6ccccc56)cc4)cc2n3-c2ccccc2)cc1 ; c1ccc(-c2ccc(N(c3ccccc3)c3ccc4c5c(-c6ccccc6)cccc5n(-c5ccccc5)c4c3)c3ccccc23)cc1 ; c1ccc(-c2cccc3c2c2ccc(N(c4ccccc4)c4ccc(-c5ccc6ccccc6c5)cc4)cc2n3-c2ccccc2)cc1 ; c1ccc(-c2cccc3c2c2ccc(N(c4ccccc4)c4ccc5c6ccccc6n(-c6ccccc6)c5c4)cc2n3-c2ccccc2)cc1]

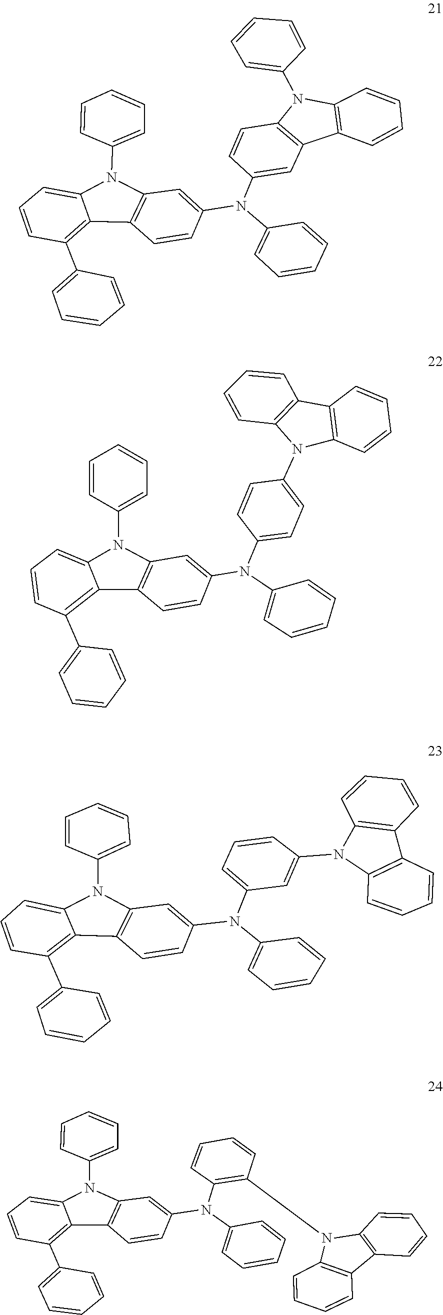 [SMILES: c1ccc(-c2cccc3c2c2ccc(N(c4ccccc4)c4ccc5c(c4)c4ccccc4n5-c4ccccc4)cc2n3-c2ccccc2)cc1 ; c1ccc(-c2cccc3c2c2ccc(N(c4ccccc4)c4ccc(-n5c6ccccc6c6ccccc65)cc4)cc2n3-c2ccccc2)cc1 ; c1ccc(-c2cccc3c2c2ccc(N(c4ccccc4)c4cccc(-n5c6ccccc6c6ccccc65)c4)cc2n3-c2ccccc2)cc1 ; c1ccc(-c2cccc3c2c2ccc(N(c4ccccc4)c4ccccc4-n4c5ccccc5c5ccccc54)cc2n3-c2ccccc2)cc1]

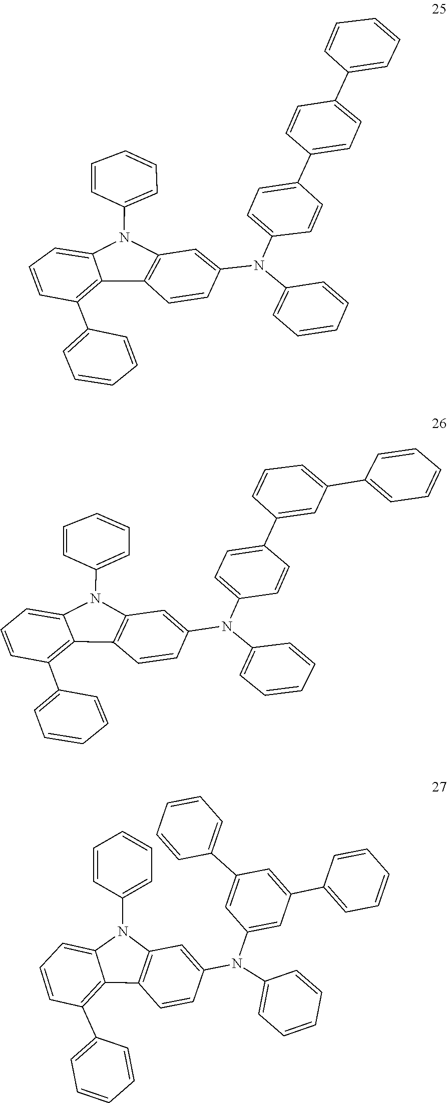 [SMILES: c1ccc(-c2ccc(-c3ccc(N(c4ccccc4)c4ccc5c6c(-c7ccccc7)cccc6n(-c6ccccc6)c5c4)cc3)cc2)cc1 ; c1ccc(-c2cccc(-c3ccc(N(c4ccccc4)c4ccc5c6c(-c7ccccc7)cccc6n(-c6ccccc6)c5c4)cc3)c2)cc1 ; c1ccc(-c2cc(-c3ccccc3)cc(N(c3ccccc3)c3ccc4c5c(-c6ccccc6)cccc5n(-c5ccccc5)c4c3)c2)cc1]

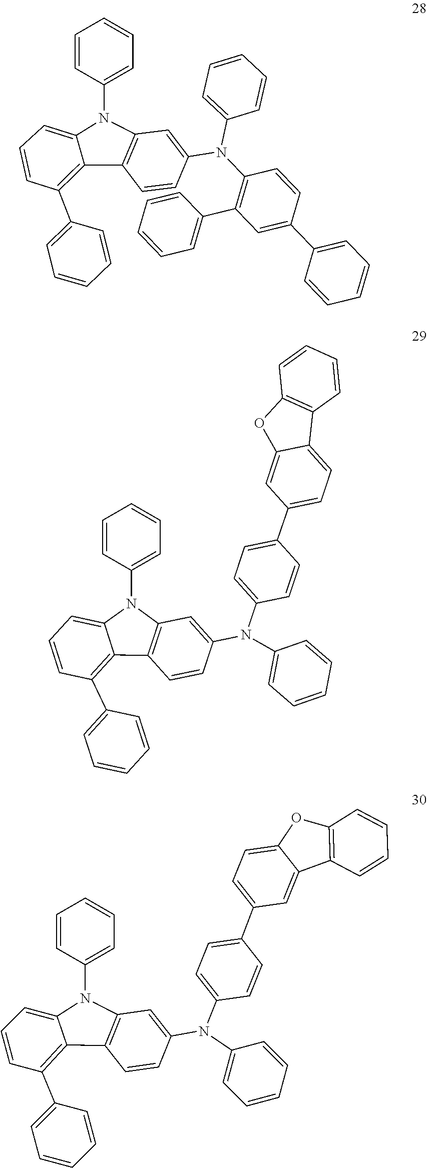

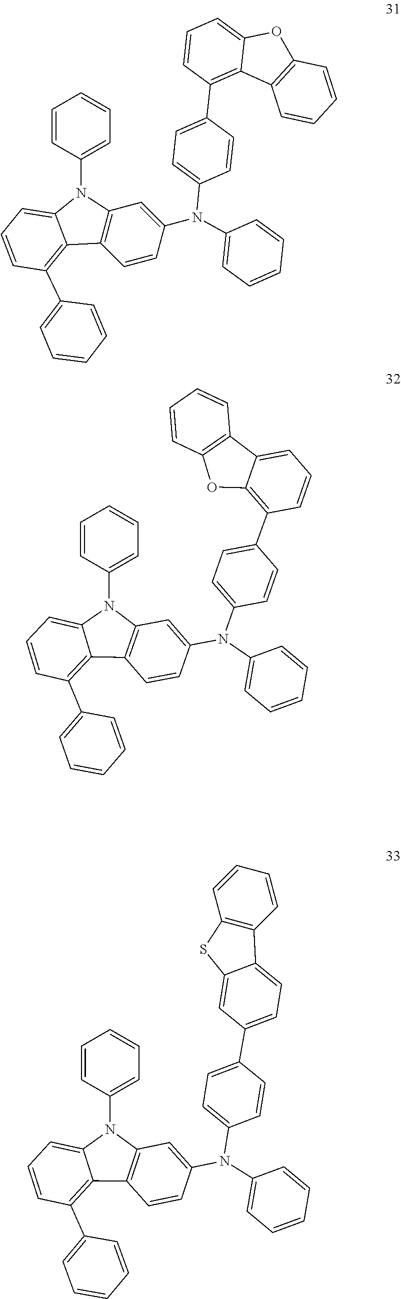 [SMILES: c1ccc(-c2cccc3c2c2ccc(N(c4ccccc4)c4ccc(-c5cccc6oc7ccccc7c56)cc4)cc2n3-c2ccccc2)cc1 ; c1ccc(-c2cccc3c2c2ccc(N(c4ccccc4)c4ccc(-c5cccc6c5oc5ccccc56)cc4)cc2n3-c2ccccc2)cc1 ; c1ccc(-c2cccc3c2c2ccc(N(c4ccccc4)c4ccc(-c5ccc6c(c5)sc5ccccc56)cc4)cc2n3-c2ccccc2)cc1]

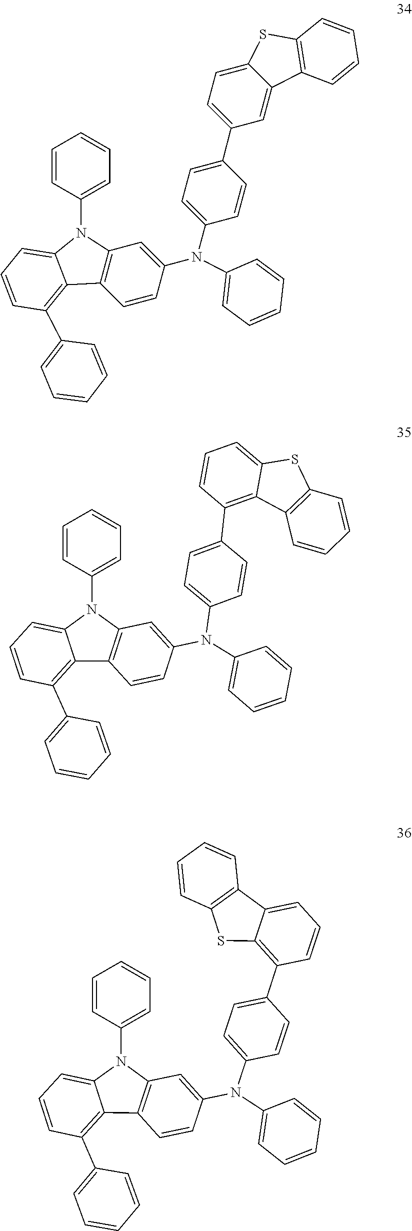 [SMILES: c1ccc(-c2cccc3c2c2ccc(N(c4ccccc4)c4ccc(-c5ccc6sc7ccccc7c6c5)cc4)cc2n3-c2ccccc2)cc1 ; c1ccc(-c2cccc3c2c2ccc(N(c4ccccc4)c4ccc(-c5cccc6sc7ccccc7c56)cc4)cc2n3-c2ccccc2)cc1 ; c1ccc(-c2cccc3c2c2ccc(N(c4ccccc4)c4ccc(-c5cccc6c5sc5ccccc56)cc4)cc2n3-c2ccccc2)cc1]

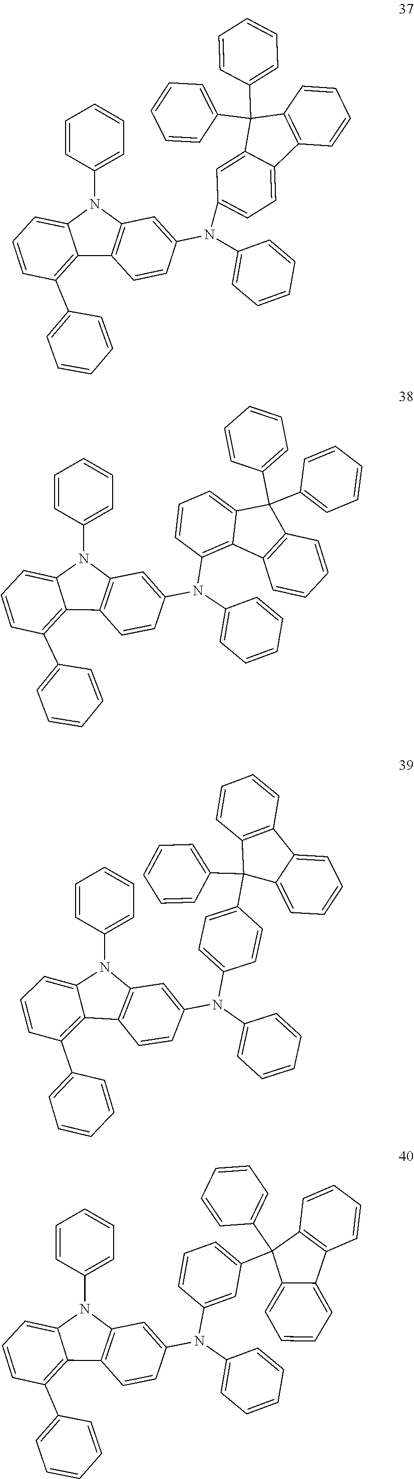 [SMILES: c1ccc(-c2cccc3c2c2ccc(N(c4ccccc4)c4ccc5c(c4)C(c4ccccc4)(c4ccccc4)c4ccccc4-5)cc2n3-c2ccccc2)cc1 ; c1ccc(-c2cccc3c2c2ccc(N(c4ccccc4)c4cccc5c4-c4ccccc4C5(c4ccccc4)c4ccccc4)cc2n3-c2ccccc2)cc1 ; c1ccc(-c2cccc3c2c2ccc(N(c4ccccc4)c4ccc(C5(c6ccccc6)c6ccccc6-c6ccccc65)cc4)cc2n3-c2ccccc2)cc1 ; c1ccc(-c2cccc3c2c2ccc(N(c4ccccc4)c4cccc(C5(c6ccccc6)c6ccccc6-c6ccccc65)c4)cc2n3-c2ccccc2)cc1]

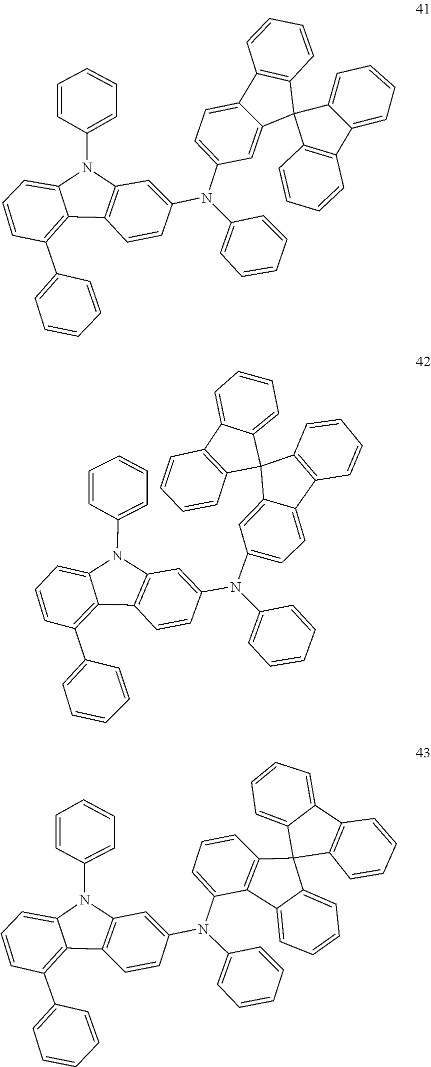 [SMILES: c1ccc(-c2cccc3c2c2ccc(N(c4ccccc4)c4ccc5c(c4)C4(c6ccccc6-c6ccccc64)c4ccccc4-5)cc2n3-c2ccccc2)cc1 ; c1ccc(-c2cccc3c2c2ccc(N(c4ccccc4)c4ccc5c(c4)C4(c6ccccc6-c6ccccc64)c4ccccc4-5)cc2n3-c2ccccc2)cc1 ; c1ccc(-c2cccc3c2c2ccc(N(c4ccccc4)c4cccc5c4-c4ccccc4C54c5ccccc5-c5ccccc54)cc2n3-c2ccccc2)cc1]

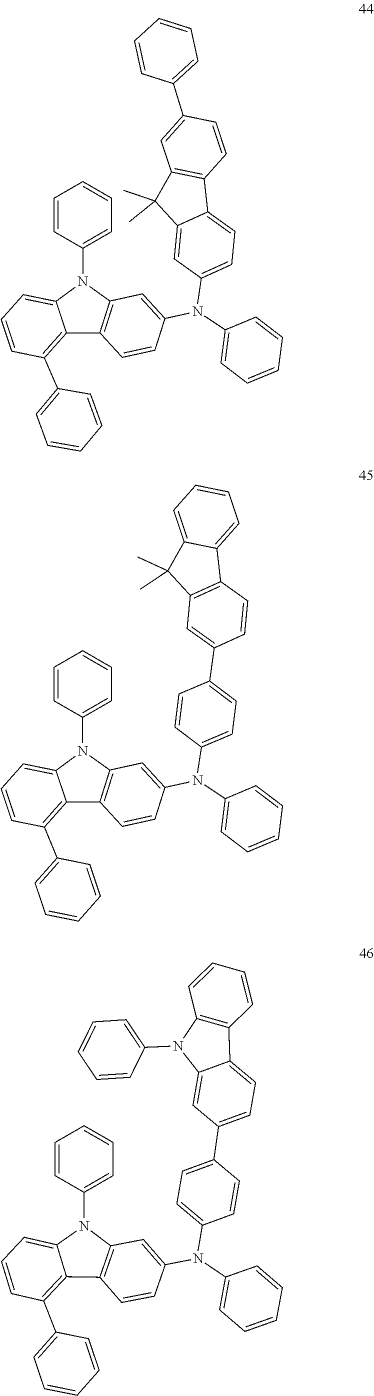 [SMILES: CC1(C)c2cc(-c3ccccc3)ccc2-c2ccc(N(c3ccccc3)c3ccc4c5c(-c6ccccc6)cccc5n(-c5ccccc5)c4c3)cc21 ; CC1(C)c2ccccc2-c2ccc(-c3ccc(N(c4ccccc4)c4ccc5c6c(-c7ccccc7)cccc6n(-c6ccccc6)c5c4)cc3)cc21 ; c1ccc(-c2cccc3c2c2ccc(N(c4ccccc4)c4ccc(-c5ccc6c7ccccc7n(-c7ccccc7)c6c5)cc4)cc2n3-c2ccccc2)cc1]

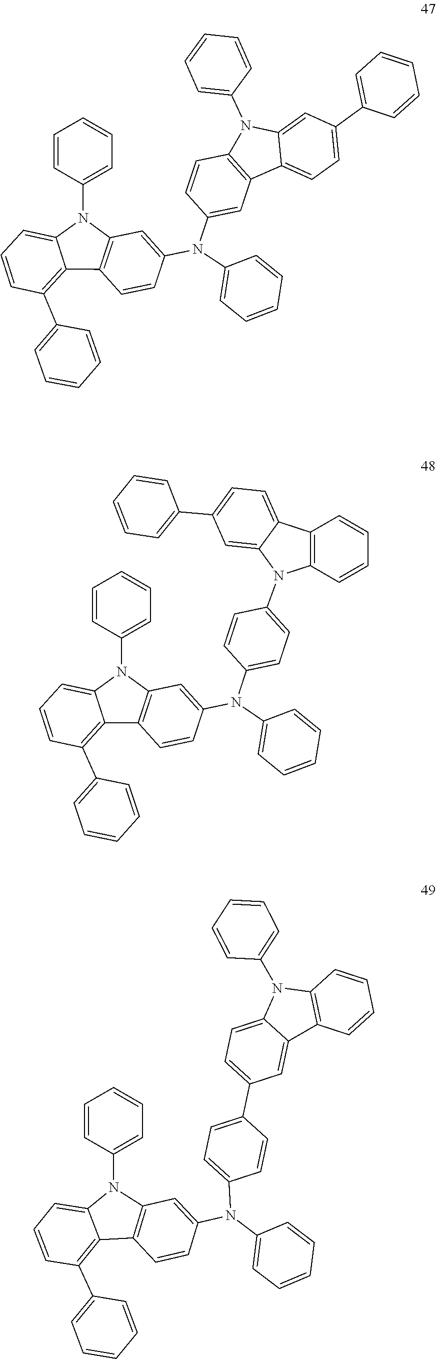

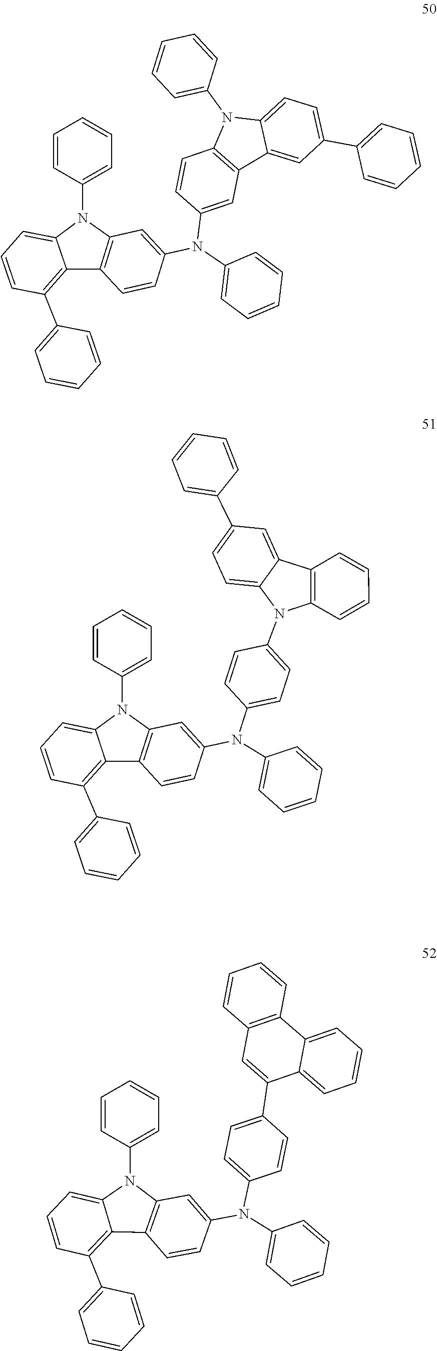 [SMILES: c1ccc(-c2ccc3c(c2)c2cc(N(c4ccccc4)c4ccc5c6c(-c7ccccc7)cccc6n(-c6ccccc6)c5c4)ccc2n3-c2ccccc2)cc1 ; c1ccc(-c2ccc3c(c2)c2ccccc2n3-c2ccc(N(c3ccccc3)c3ccc4c5c(-c6ccccc6)cccc5n(-c5ccccc5)c4c3)cc2)cc1 ; c1ccc(-c2cccc3c2c2ccc(N(c4ccccc4)c4ccc(-c5cc6ccccc6c6ccccc56)cc4)cc2n3-c2ccccc2)cc1]

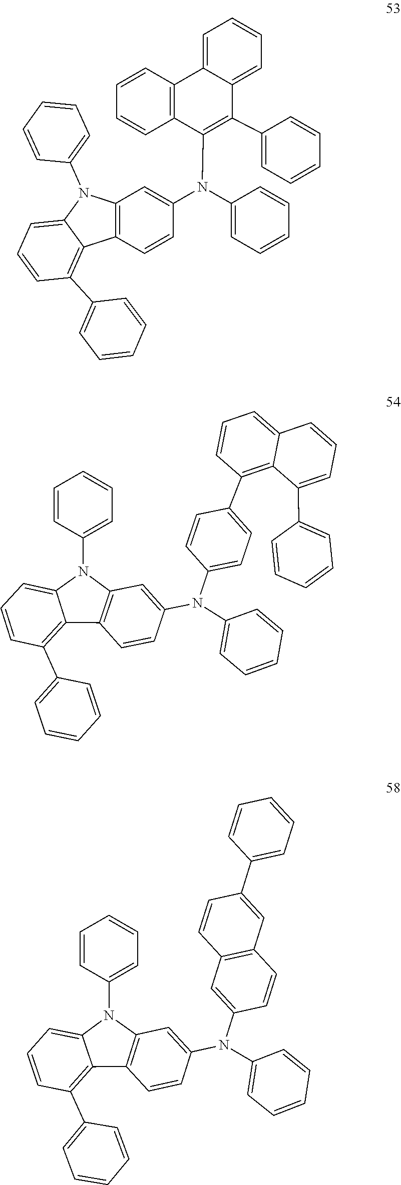 [SMILES: c1ccc(-c2c(N(c3ccccc3)c3ccc4c5c(-c6ccccc6)cccc5n(-c5ccccc5)c4c3)c3ccccc3c3ccccc23)cc1 ; c1ccc(-c2cccc3cccc(-c4ccc(N(c5ccccc5)c5ccc6c7c(-c8ccccc8)cccc7n(-c7ccccc7)c6c5)cc4)c23)cc1 ; c1ccc(-c2ccc3cc(N(c4ccccc4)c4ccc5c6c(-c7ccccc7)cccc6n(-c6ccccc6)c5c4)ccc3c2)cc1]

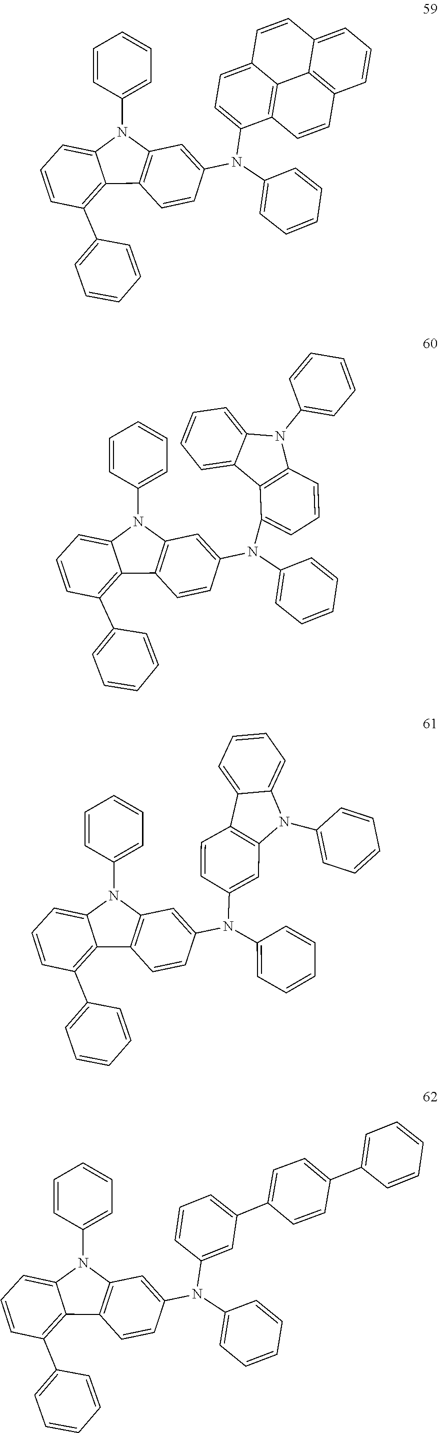 [SMILES: c1ccc(-c2cccc3c2c2ccc(N(c4ccccc4)c4ccc5ccc6cccc7ccc4c5c67)cc2n3-c2ccccc2)cc1 ; c1ccc(-c2cccc3c2c2ccc(N(c4ccccc4)c4cccc5c4c4ccccc4n5-c4ccccc4)cc2n3-c2ccccc2)cc1 ; c1ccc(-c2cccc3c2c2ccc(N(c4ccccc4)c4ccc5c6ccccc6n(-c6ccccc6)c5c4)cc2n3-c2ccccc2)cc1 ; c1ccc(-c2ccc(-c3cccc(N(c4ccccc4)c4ccc5c6c(-c7ccccc7)cccc6n(-c6ccccc6)c5c4)c3)cc2)cc1]

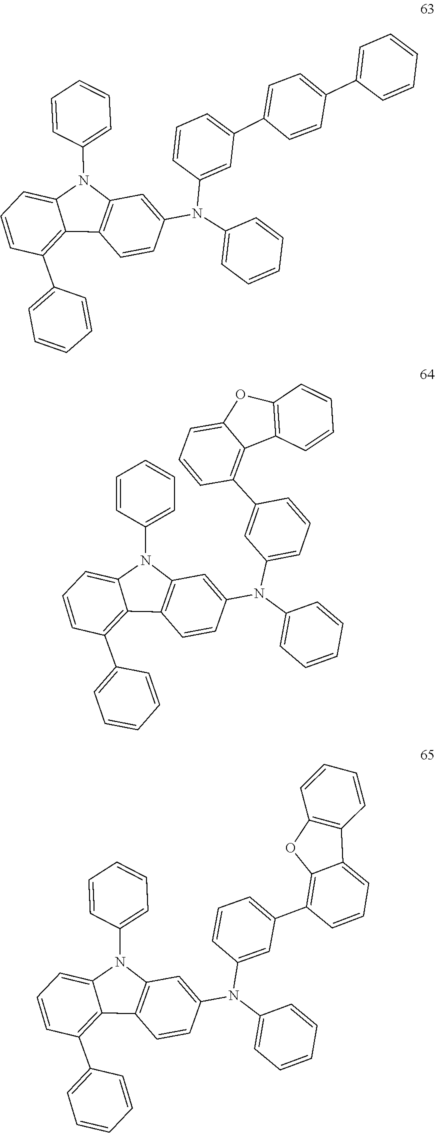 [SMILES: c1ccc(-c2ccc(-c3cccc(N(c4ccccc4)c4ccc5c6c(-c7ccccc7)cccc6n(-c6ccccc6)c5c4)c3)cc2)cc1 ; c1ccc(-c2cccc3c2c2ccc(N(c4ccccc4)c4cccc(-c5cccc6oc7ccccc7c56)c4)cc2n3-c2ccccc2)cc1 ; c1ccc(-c2cccc3c2c2ccc(N(c4ccccc4)c4cccc(-c5cccc6c5oc5ccccc56)c4)cc2n3-c2ccccc2)cc1]

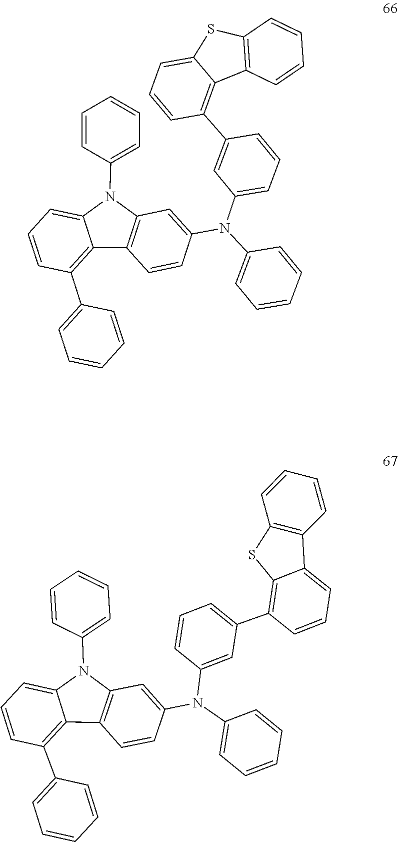 [SMILES: c1ccc(-c2cccc3c2c2ccc(N(c4ccccc4)c4cccc(-c5cccc6sc7ccccc7c56)c4)cc2n3-c2ccccc2)cc1 ; c1ccc(-c2cccc3c2c2ccc(N(c4ccccc4)c4cccc(-c5cccc6c5sc5ccccc56)c4)cc2n3-c2ccccc2)cc1]

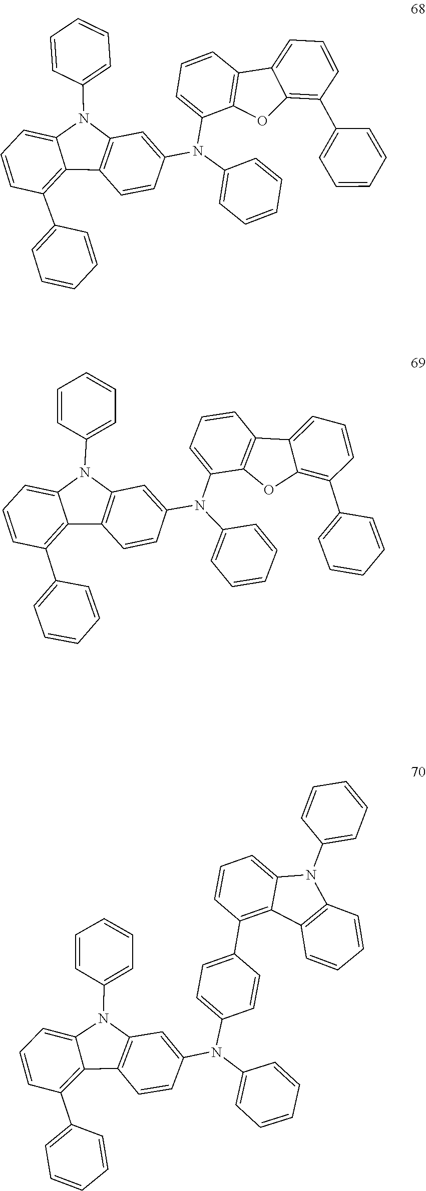

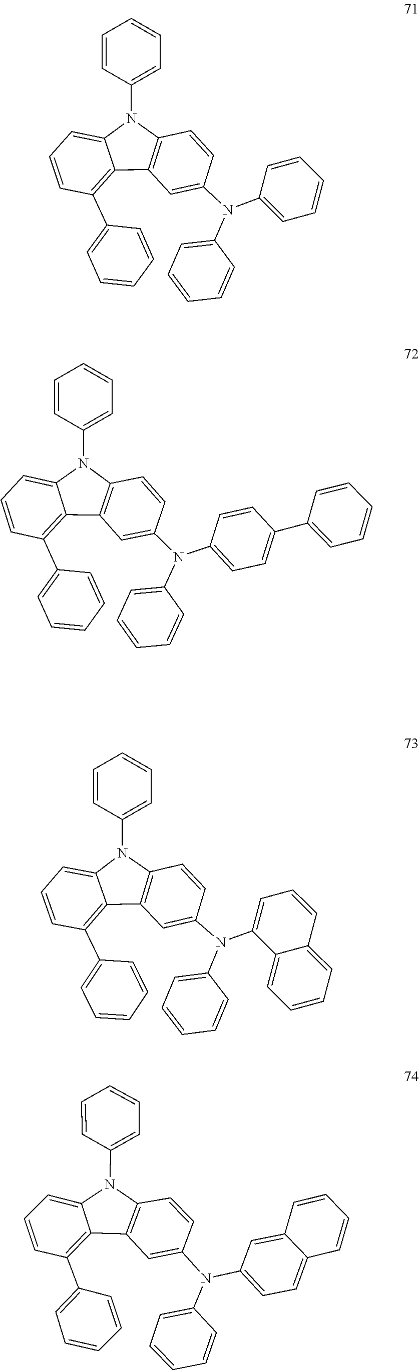 [SMILES: c1ccc(-c2cccc3c2c2cc(N(c4ccccc4)c4ccccc4)ccc2n3-c2ccccc2)cc1 ; c1ccc(-c2ccc(N(c3ccccc3)c3ccc4c(c3)c3c(-c5ccccc5)cccc3n4-c3ccccc3)cc2)cc1 ; c1ccc(-c2cccc3c2c2cc(N(c4ccccc4)c4cccc5ccccc45)ccc2n3-c2ccccc2)cc1 ; c1ccc(-c2cccc3c2c2cc(N(c4ccccc4)c4ccc5ccccc5c4)ccc2n3-c2ccccc2)cc1]

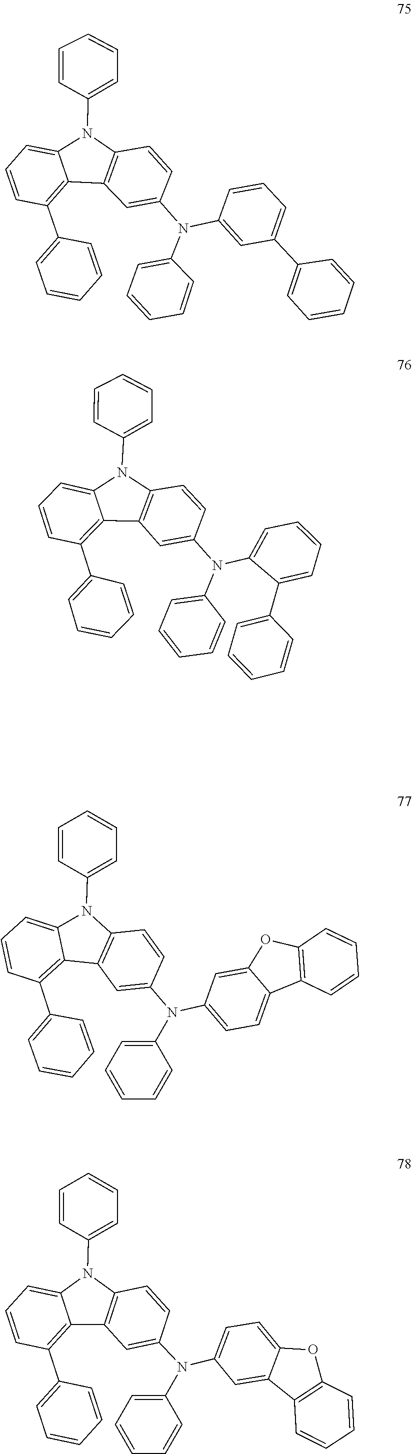 [SMILES: c1ccc(-c2cccc(N(c3ccccc3)c3ccc4c(c3)c3c(-c5ccccc5)cccc3n4-c3ccccc3)c2)cc1 ; c1ccc(-c2ccccc2N(c2ccccc2)c2ccc3c(c2)c2c(-c4ccccc4)cccc2n3-c2ccccc2)cc1 ; c1ccc(-c2cccc3c2c2cc(N(c4ccccc4)c4ccc5c(c4)oc4ccccc45)ccc2n3-c2ccccc2)cc1 ; c1ccc(-c2cccc3c2c2cc(N(c4ccccc4)c4ccc5oc6ccccc6c5c4)ccc2n3-c2ccccc2)cc1]

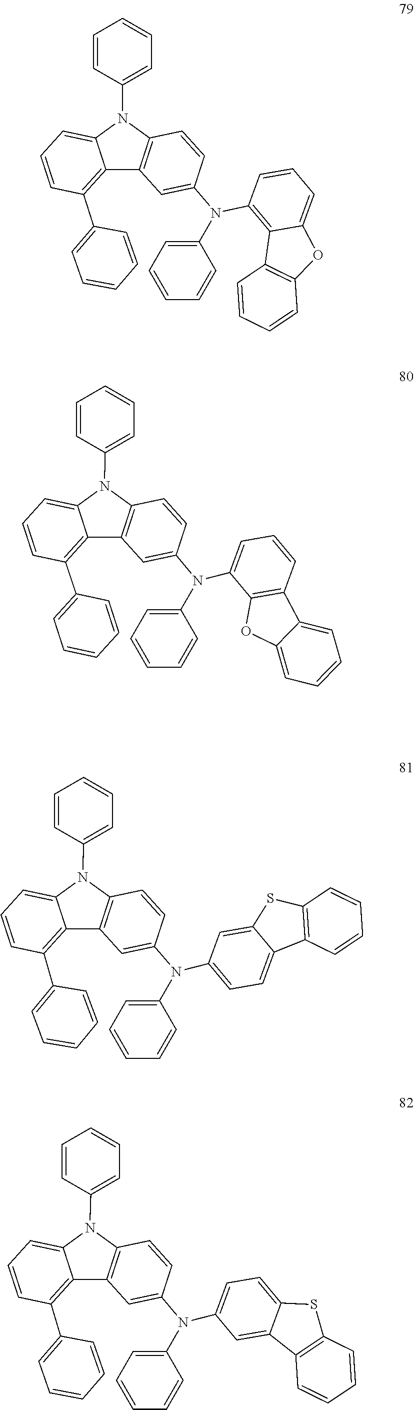 [SMILES: c1ccc(-c2cccc3c2c2cc(N(c4ccccc4)c4cccc5oc6ccccc6c45)ccc2n3-c2ccccc2)cc1 ; c1ccc(-c2cccc3c2c2cc(N(c4ccccc4)c4cccc5c4oc4ccccc45)ccc2n3-c2ccccc2)cc1 ; c1ccc(-c2cccc3c2c2cc(N(c4ccccc4)c4ccc5c(c4)sc4ccccc45)ccc2n3-c2ccccc2)cc1 ; c1ccc(-c2cccc3c2c2cc(N(c4ccccc4)c4ccc5sc6ccccc6c5c4)ccc2n3-c2ccccc2)cc1]

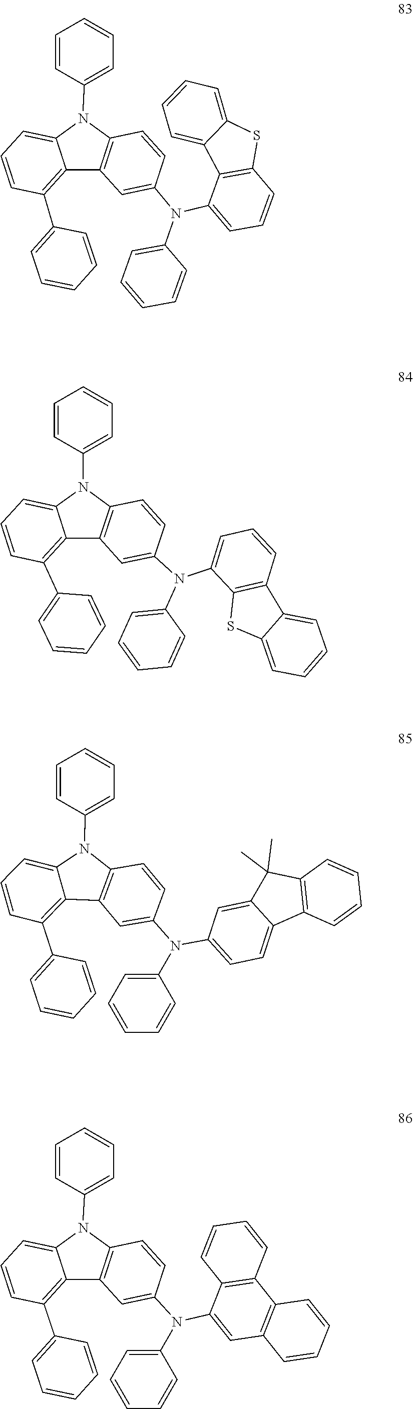 [SMILES: c1ccc(-c2cccc3c2c2cc(N(c4ccccc4)c4cccc5sc6ccccc6c45)ccc2n3-c2ccccc2)cc1 ; c1ccc(-c2cccc3c2c2cc(N(c4ccccc4)c4cccc5c4sc4ccccc45)ccc2n3-c2ccccc2)cc1 ; CC1(C)c2ccccc2-c2ccc(N(c3ccccc3)c3ccc4c(c3)c3c(-c5ccccc5)cccc3n4-c3ccccc3)cc21 ; c1ccc(-c2cccc3c2c2cc(N(c4ccccc4)c4cc5ccccc5c5ccccc45)ccc2n3-c2ccccc2)cc1]

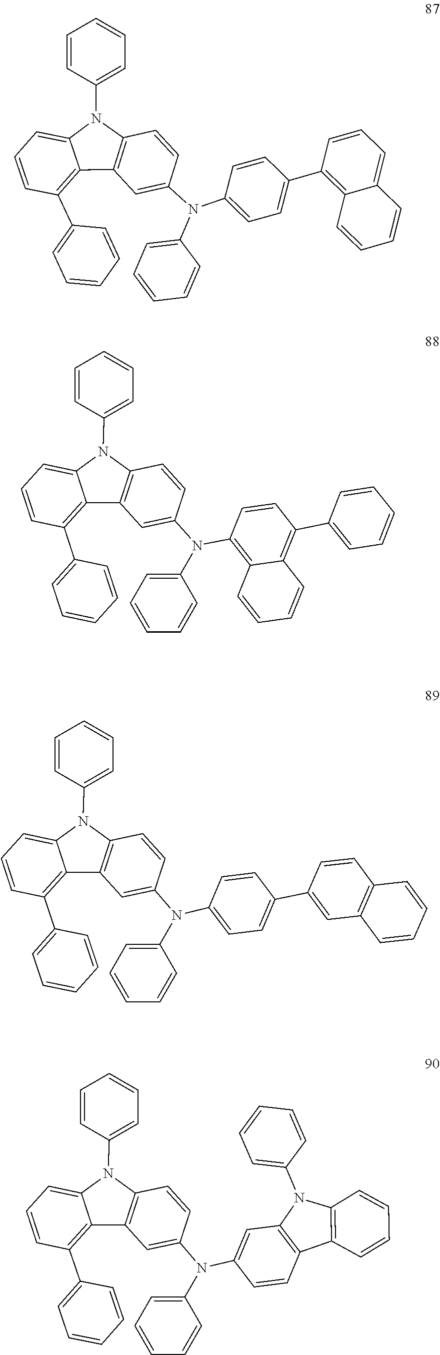 [SMILES: c1ccc(-c2cccc3c2c2cc(N(c4ccccc4)c4ccc(-c5cccc6ccccc56)cc4)ccc2n3-c2ccccc2)cc1 ; c1ccc(-c2ccc(N(c3ccccc3)c3ccc4c(c3)c3c(-c5ccccc5)cccc3n4-c3ccccc3)c3ccccc23)cc1 ; c1ccc(-c2cccc3c2c2cc(N(c4ccccc4)c4ccc(-c5ccc6ccccc6c5)cc4)ccc2n3-c2ccccc2)cc1 ; c1ccc(-c2cccc3c2c2cc(N(c4ccccc4)c4ccc5c6ccccc6n(-c6ccccc6)c5c4)ccc2n3-c2ccccc2)cc1]

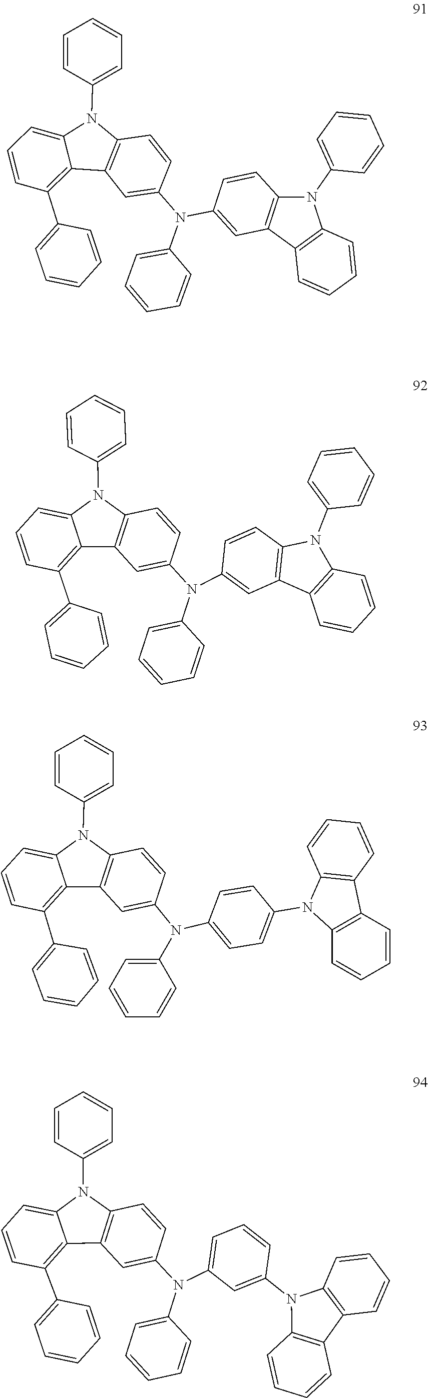 [SMILES: c1ccc(-c2cccc3c2c2cc(N(c4ccccc4)c4ccc5c(c4)c4ccccc4n5-c4ccccc4)ccc2n3-c2ccccc2)cc1 ; c1ccc(-c2cccc3c2c2cc(N(c4ccccc4)c4ccc5c(c4)c4ccccc4n5-c4ccccc4)ccc2n3-c2ccccc2)cc1 ; c1ccc(-c2cccc3c2c2cc(N(c4ccccc4)c4ccc(-n5c6ccccc6c6ccccc65)cc4)ccc2n3-c2ccccc2)cc1 ; c1ccc(-c2cccc3c2c2cc(N(c4ccccc4)c4cccc(-n5c6ccccc6c6ccccc65)c4)ccc2n3-c2ccccc2)cc1]

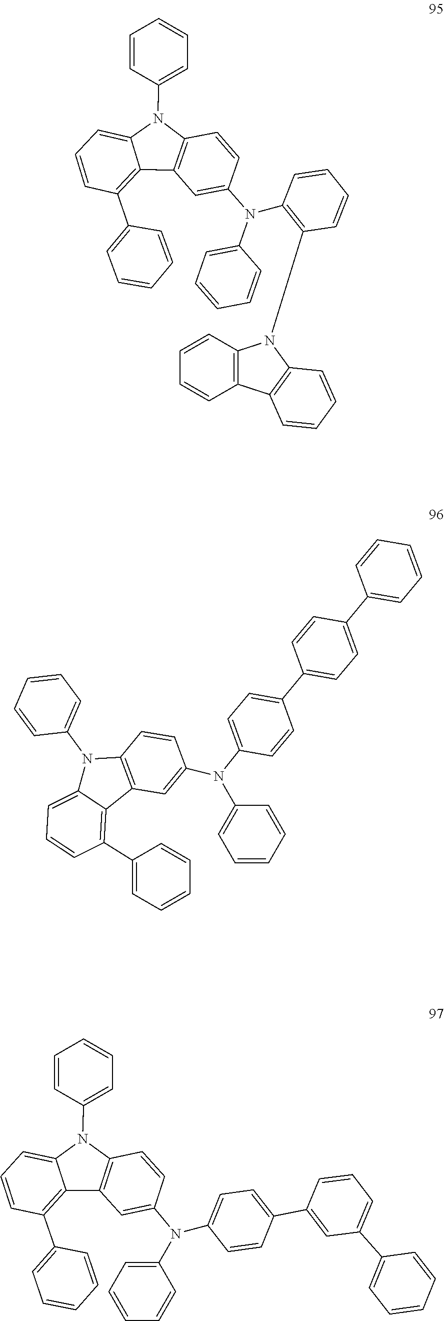 [SMILES: c1ccc(-c2cccc3c2c2cc(N(c4ccccc4)c4ccccc4-n4c5ccccc5c5ccccc54)ccc2n3-c2ccccc2)cc1 ; c1ccc(-c2ccc(-c3ccc(N(c4ccccc4)c4ccc5c(c4)c4c(-c6ccccc6)cccc4n5-c4ccccc4)cc3)cc2)cc1 ; c1ccc(-c2cccc(-c3ccc(N(c4ccccc4)c4ccc5c(c4)c4c(-c6ccccc6)cccc4n5-c4ccccc4)cc3)c2)cc1]

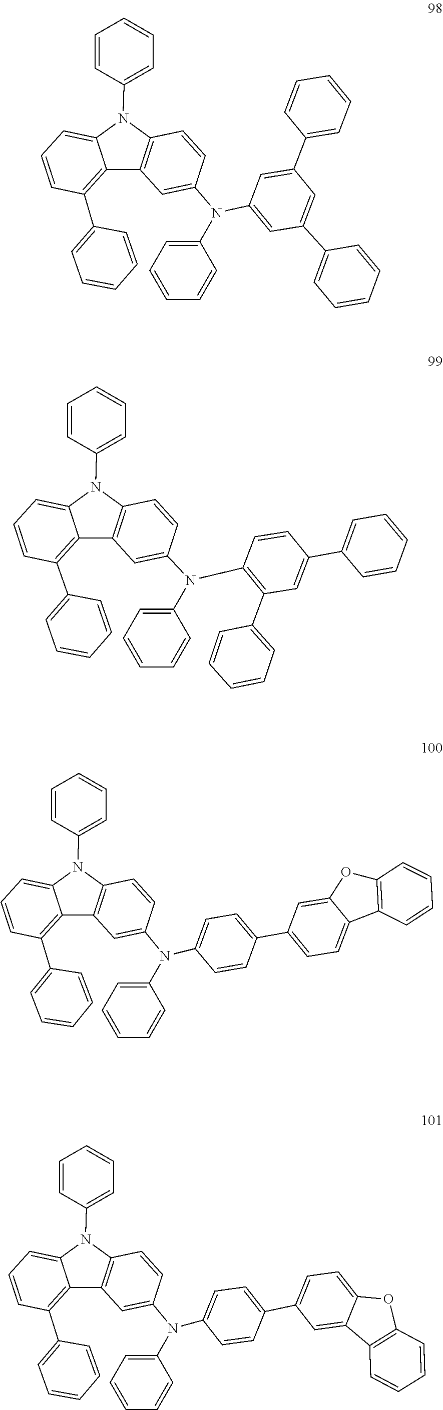 [SMILES: c1ccc(-c2cc(-c3ccccc3)cc(N(c3ccccc3)c3ccc4c(c3)c3c(-c5ccccc5)cccc3n4-c3ccccc3)c2)cc1 ; c1ccc(-c2ccc(N(c3ccccc3)c3ccc4c(c3)c3c(-c5ccccc5)cccc3n4-c3ccccc3)c(-c3ccccc3)c2)cc1 ; c1ccc(-c2cccc3c2c2cc(N(c4ccccc4)c4ccc(-c5ccc6c(c5)oc5ccccc56)cc4)ccc2n3-c2ccccc2)cc1 ; c1ccc(-c2cccc3c2c2cc(N(c4ccccc4)c4ccc(-c5ccc6oc7ccccc7c6c5)cc4)ccc2n3-c2ccccc2)cc1]

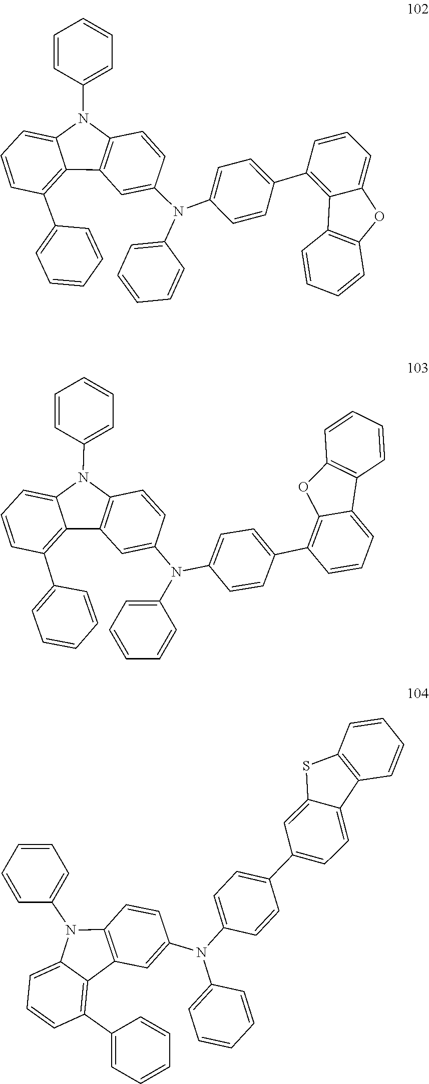 [SMILES: c1ccc(-c2cccc3c2c2cc(N(c4ccccc4)c4ccc(-c5cccc6oc7ccccc7c56)cc4)ccc2n3-c2ccccc2)cc1 ; c1ccc(-c2cccc3c2c2cc(N(c4ccccc4)c4ccc(-c5cccc6c5oc5ccccc56)cc4)ccc2n3-c2ccccc2)cc1 ; c1ccc(-c2cccc3c2c2cc(N(c4ccccc4)c4ccc(-c5ccc6c(c5)sc5ccccc56)cc4)ccc2n3-c2ccccc2)cc1]

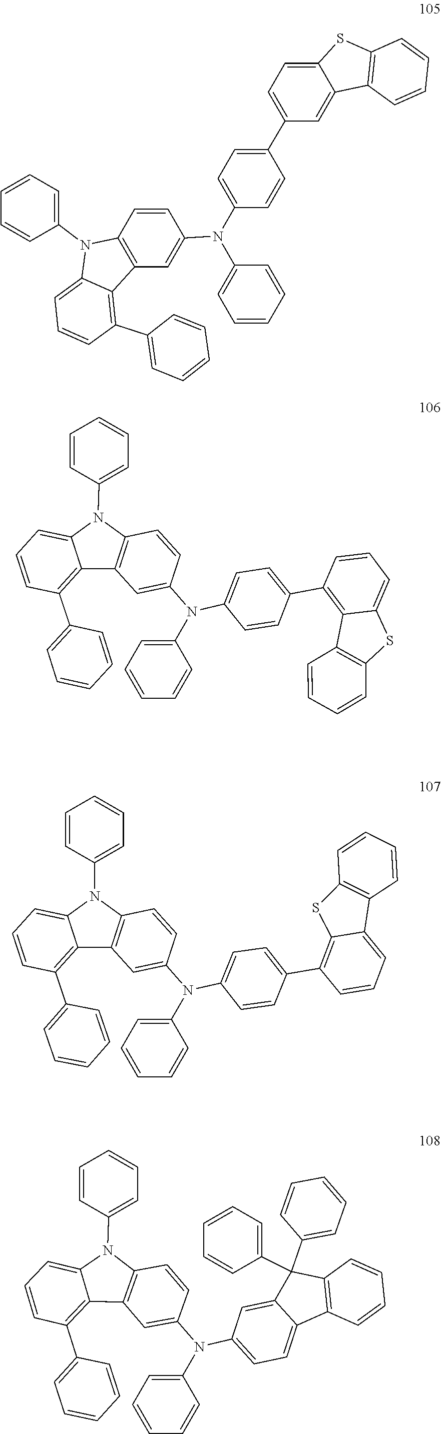 [SMILES: c1ccc(-c2cccc3c2c2cc(N(c4ccccc4)c4ccc(-c5ccc6sc7ccccc7c6c5)cc4)ccc2n3-c2ccccc2)cc1 ; c1ccc(-c2cccc3c2c2cc(N(c4ccccc4)c4ccc(-c5cccc6sc7ccccc7c56)cc4)ccc2n3-c2ccccc2)cc1 ; c1ccc(-c2cccc3c2c2cc(N(c4ccccc4)c4ccc(-c5cccc6c5sc5ccccc56)cc4)ccc2n3-c2ccccc2)cc1 ; c1ccc(-c2cccc3c2c2cc(N(c4ccccc4)c4ccc5c(c4)C(c4ccccc4)(c4ccccc4)c4ccccc4-5)ccc2n3-c2ccccc2)cc1]

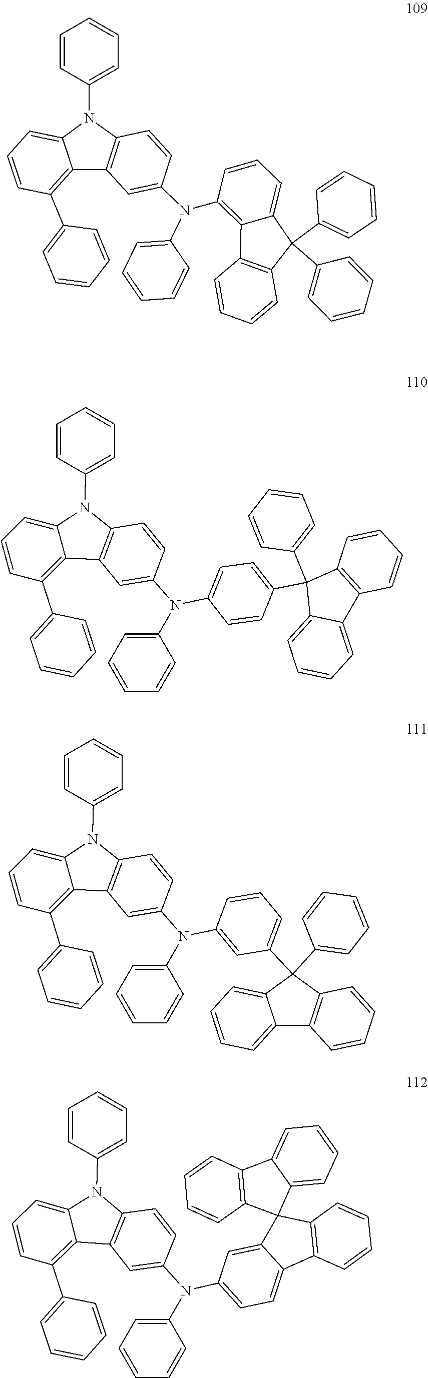 [SMILES: c1ccc(-c2cccc3c2c2cc(N(c4ccccc4)c4cccc5c4-c4ccccc4C5(c4ccccc4)c4ccccc4)ccc2n3-c2ccccc2)cc1 ; c1ccc(-c2cccc3c2c2cc(N(c4ccccc4)c4ccc(C5(c6ccccc6)c6ccccc6-c6ccccc65)cc4)ccc2n3-c2ccccc2)cc1 ; c1ccc(-c2cccc3c2c2cc(N(c4ccccc4)c4cccc(C5(c6ccccc6)c6ccccc6-c6ccccc65)c4)ccc2n3-c2ccccc2)cc1 ; c1ccc(-c2cccc3c2c2cc(N(c4ccccc4)c4ccc5c(c4)C4(c6ccccc6-c6ccccc64)c4ccccc4-5)ccc2n3-c2ccccc2)cc1]

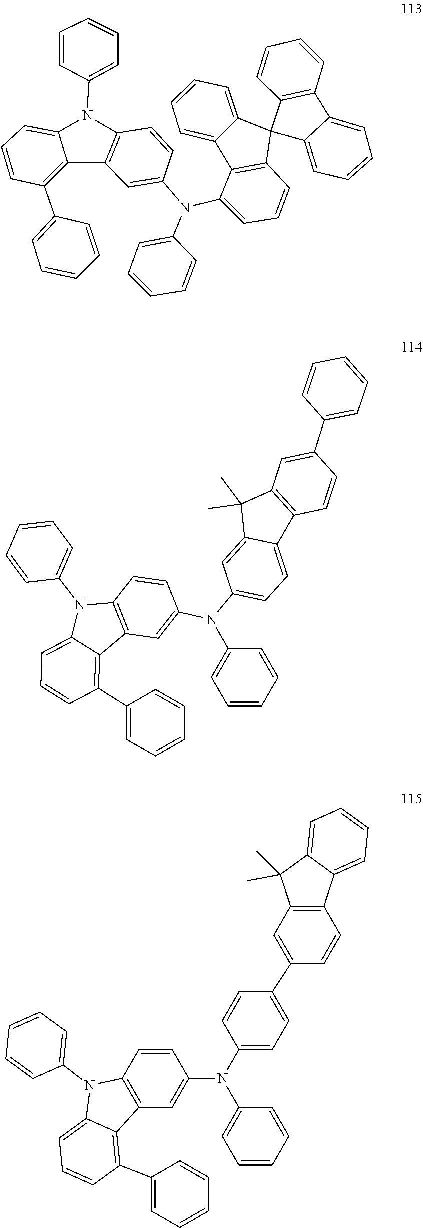 [SMILES: c1ccc(-c2cccc3c2c2cc(N(c4ccccc4)c4cccc5c4-c4ccccc4C54c5ccccc5-c5ccccc54)ccc2n3-c2ccccc2)cc1 ; CC1(C)c2cc(-c3ccccc3)ccc2-c2ccc(N(c3ccccc3)c3ccc4c(c3)c3c(-c5ccccc5)cccc3n4-c3ccccc3)cc21 ; CC1(C)c2ccccc2-c2ccc(-c3ccc(N(c4ccccc4)c4ccc5c(c4)c4c(-c6ccccc6)cccc4n5-c4ccccc4)cc3)cc21]

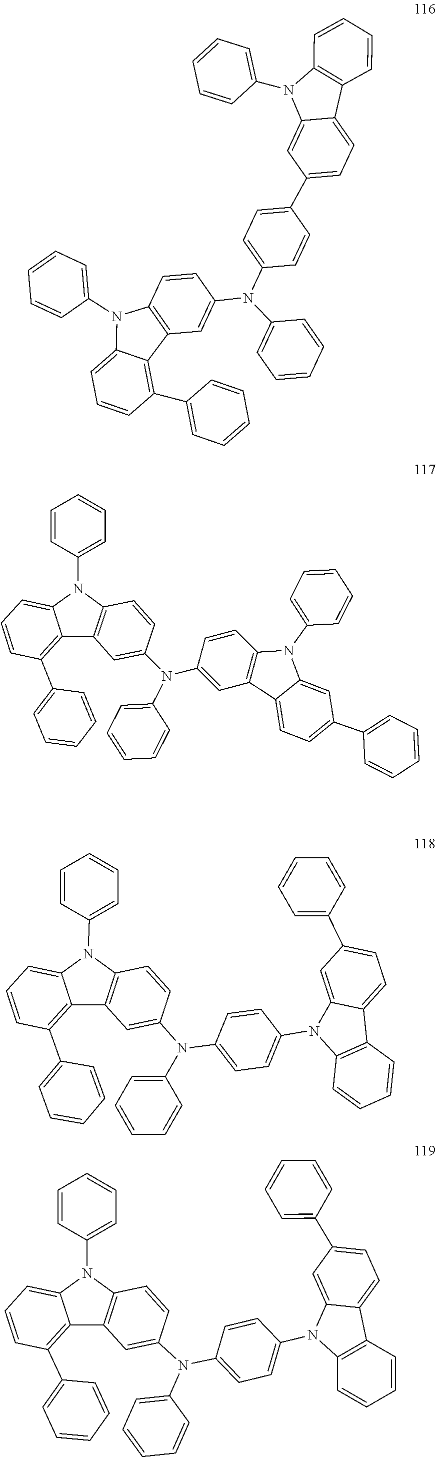 [SMILES: c1ccc(-c2cccc3c2c2cc(N(c4ccccc4)c4ccc(-c5ccc6c7ccccc7n(-c7ccccc7)c6c5)cc4)ccc2n3-c2ccccc2)cc1 ; c1ccc(-c2ccc3c4cc(N(c5ccccc5)c5ccc6c(c5)c5c(-c7ccccc7)cccc5n6-c5ccccc5)ccc4n(-c4ccccc4)c3c2)cc1 ; c1ccc(-c2ccc3c4ccccc4n(-c4ccc(N(c5ccccc5)c5ccc6c(c5)c5c(-c7ccccc7)cccc5n6-c5ccccc5)cc4)c3c2)cc1 ; c1ccc(-c2ccc3c4ccccc4n(-c4ccc(N(c5ccccc5)c5ccc6c(c5)c5c(-c7ccccc7)cccc5n6-c5ccccc5)cc4)c3c2)cc1]

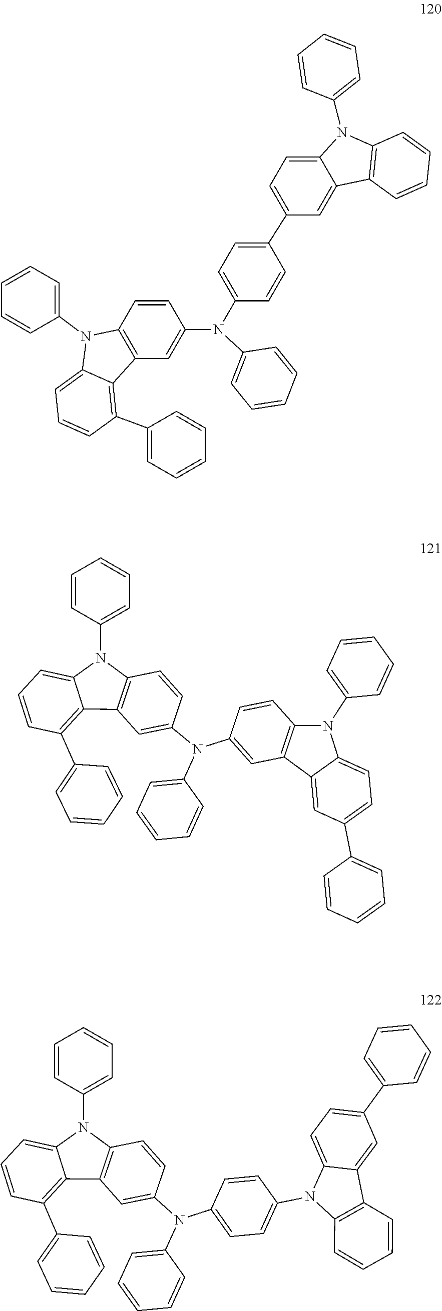 [SMILES: c1ccc(-c2cccc3c2c2cc(N(c4ccccc4)c4ccc(-c5ccc6c(c5)c5ccccc5n6-c5ccccc5)cc4)ccc2n3-c2ccccc2)cc1 ; c1ccc(-c2ccc3c(c2)c2cc(N(c4ccccc4)c4ccc5c(c4)c4c(-c6ccccc6)cccc4n5-c4ccccc4)ccc2n3-c2ccccc2)cc1 ; c1ccc(-c2ccc3c(c2)c2ccccc2n3-c2ccc(N(c3ccccc3)c3ccc4c(c3)c3c(-c5ccccc5)cccc3n4-c3ccccc3)cc2)cc1]

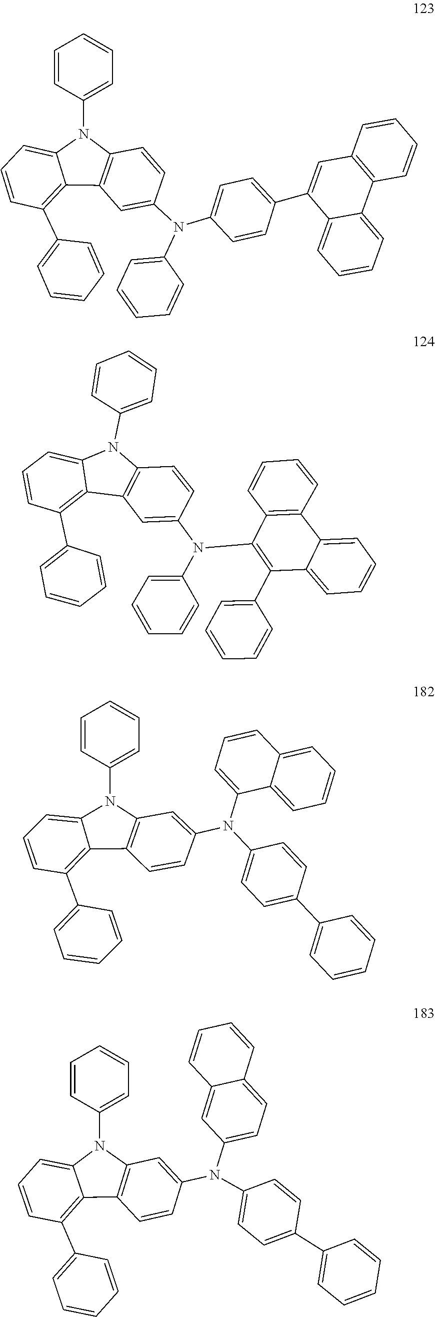

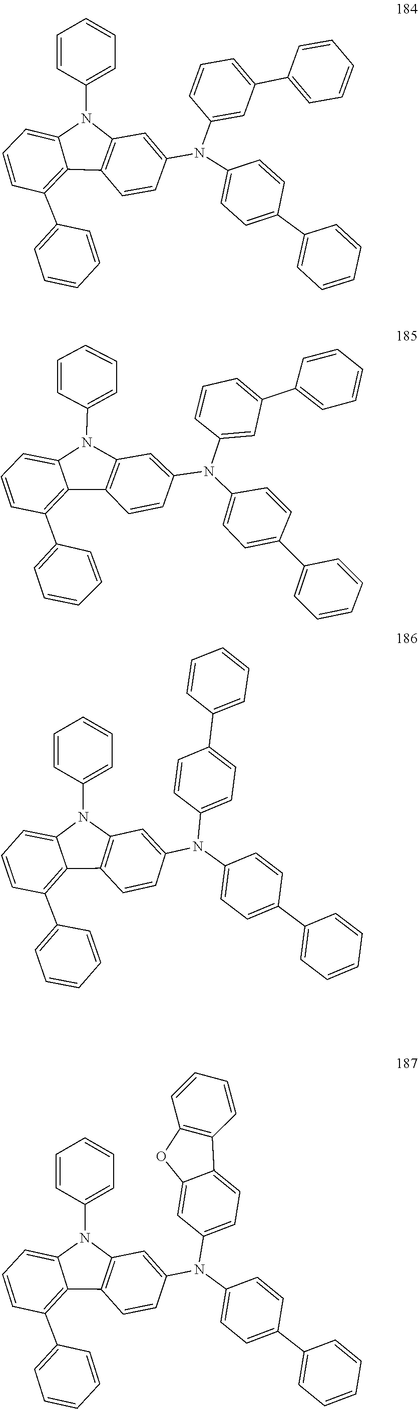 [SMILES: c1ccc(-c2ccc(N(c3cccc(-c4ccccc4)c3)c3ccc4c5c(-c6ccccc6)cccc5n(-c5ccccc5)c4c3)cc2)cc1 ; c1ccc(-c2ccc(N(c3cccc(-c4ccccc4)c3)c3ccc4c5c(-c6ccccc6)cccc5n(-c5ccccc5)c4c3)cc2)cc1 ; c1ccc(-c2ccc(N(c3ccc(-c4ccccc4)cc3)c3ccc4c5c(-c6ccccc6)cccc5n(-c5ccccc5)c4c3)cc2)cc1 ; c1ccc(-c2ccc(N(c3ccc4c(c3)oc3ccccc34)c3ccc4c5c(-c6ccccc6)cccc5n(-c5ccccc5)c4c3)cc2)cc1]

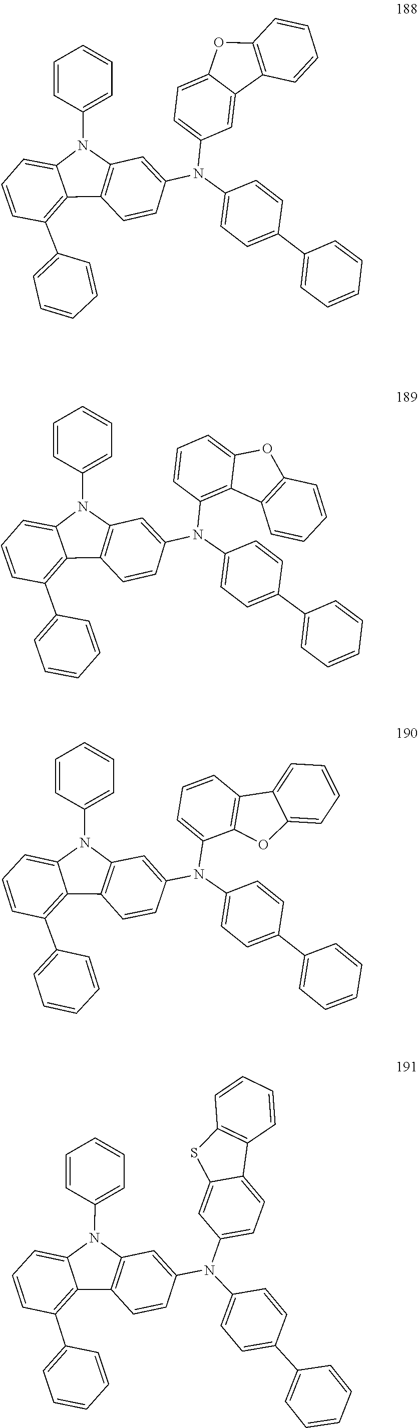 [SMILES: c1ccc(-c2ccc(N(c3ccc4oc5ccccc5c4c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3cccc4oc5ccccc5c34)cc2)cc1 ; c1ccc(-c2ccc(N(c3ccc4c5c(-c6ccccc6)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6ccccc6)cccc5n(-c5ccccc5)c4c3)cc2)cc1]

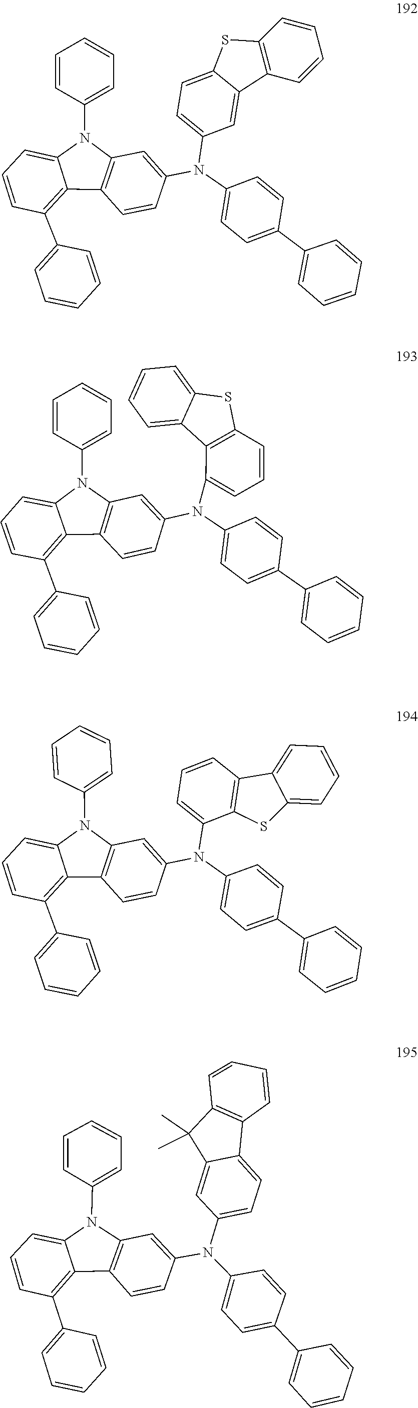 [SMILES: c1ccc(-c2ccc(N(c3ccc4sc5ccccc5c4c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3cccc4sc5ccccc5c34)cc2)cc1 ; c1ccc(-c2ccc(N(c3ccc4c5c(-c6ccccc6)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6ccccc6)cccc5n(-c5ccccc5)c4c3)cc21]

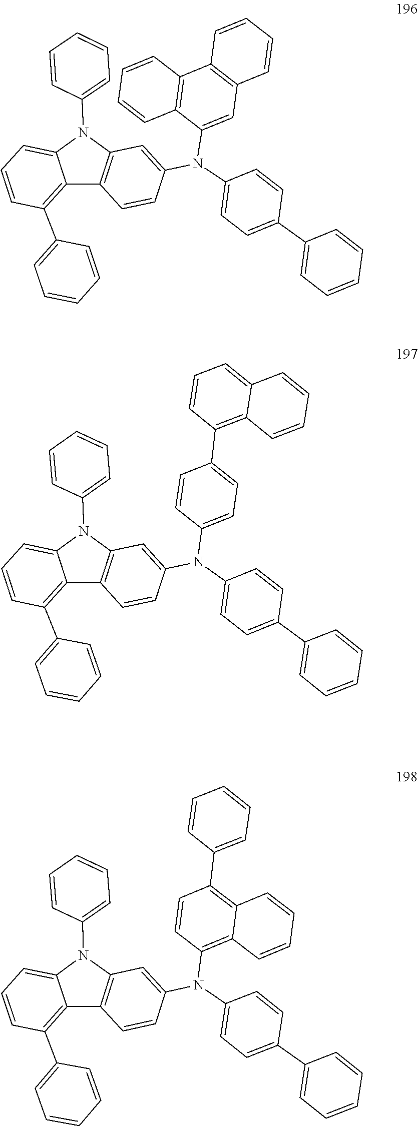 [SMILES: c1ccc(-c2ccc(N(c3ccc4c5c(-c6ccccc6)cccc5n(-c5ccccc5)c4c3)c3cc4ccccc4c4ccccc34)cc2)cc1 ; c1ccc(-c2ccc(N(c3ccc(-c4cccc5ccccc45)cc3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3ccc(-c4ccccc4)c4ccccc34)cc2)cc1]

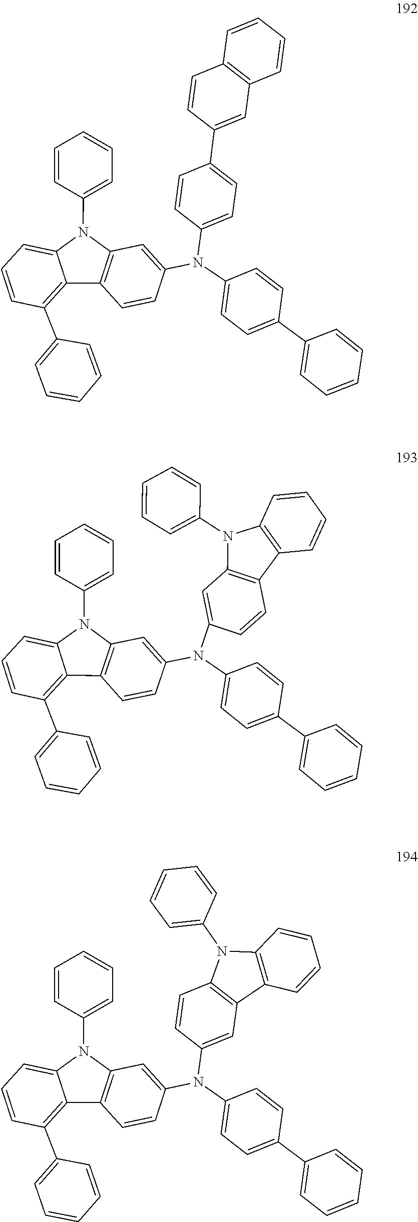 [SMILES: c1ccc(-c2ccc(N(c3ccc(-c4ccc5ccccc5c4)cc3)c3ccc4c5c(-c6ccccc6)cccc5n(-c5ccccc5)c4c3)cc2)cc1 ; c1ccc(-c2ccc(N(c3ccc4c5ccccc5n(-c5ccccc5)c4c3)c3ccc4c5c(-c6ccccc6)cccc5n(-c5ccccc5)c4c3)cc2)cc1 ; c1ccc(-c2ccc(N(c3ccc4c(c3)c3ccccc3n4-c3ccccc3)c3ccc4c5c(-c6ccccc6)cccc5n(-c5ccccc5)c4c3)cc2)cc1]

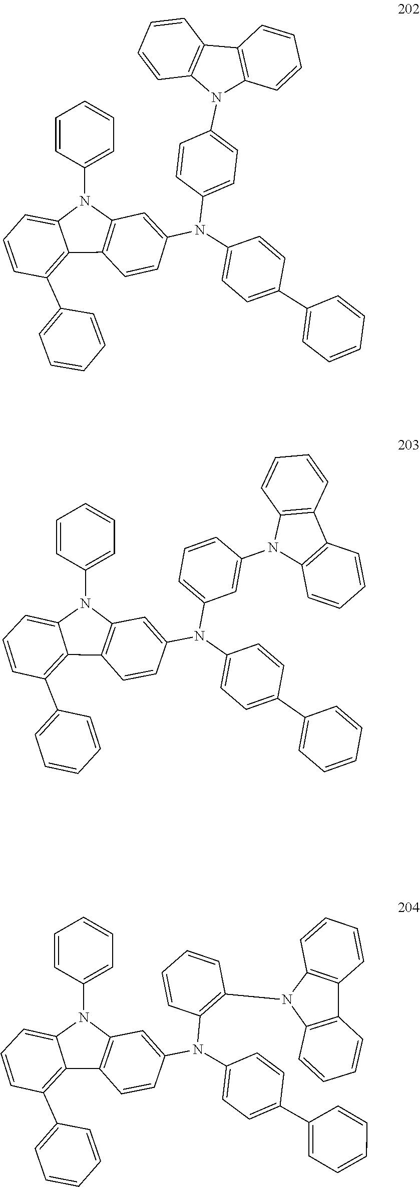 [SMILES: c1ccc(-c2ccc(N(c3ccc(-n4c5ccccc5c5ccccc54)cc3)c3ccc4c5c(-c6ccccc6)cccc5n(-c5ccccc5)c4c3)cc2)cc1 ; c1ccc(-c2ccc(N(c3cccc(-n4c5ccccc5c5ccccc54)c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3ccccc3-n3c4ccccc4c4ccccc43)cc2)cc1]

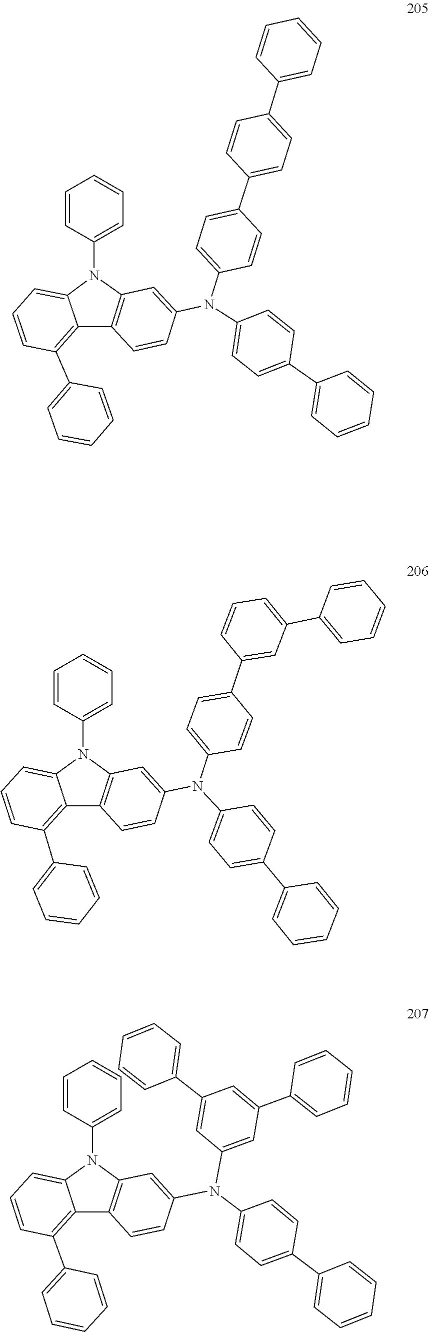 [SMILES: c1ccc(-c2ccc(-c3ccc(N(c4ccc(-c5ccccc5)cc4)c4ccc5c6c(-c7ccccc7)cccc6n(-c6ccccc6)c5c4)cc3)cc2)cc1 ; c1ccc(-c2ccc(N(c3ccc(-c4cccc(-c5ccccc5)c4)cc3)c3ccc4c5c(-c6ccccc6)cccc5n(-c5ccccc5)c4c3)cc2)cc1 ; c1ccc(-c2ccc(N(c3cc(-c4ccccc4)cc(-c4ccccc4)c3)c3ccc4c5c(-c6ccccc6)cccc5n(-c5ccccc5)c4c3)cc2)cc1]

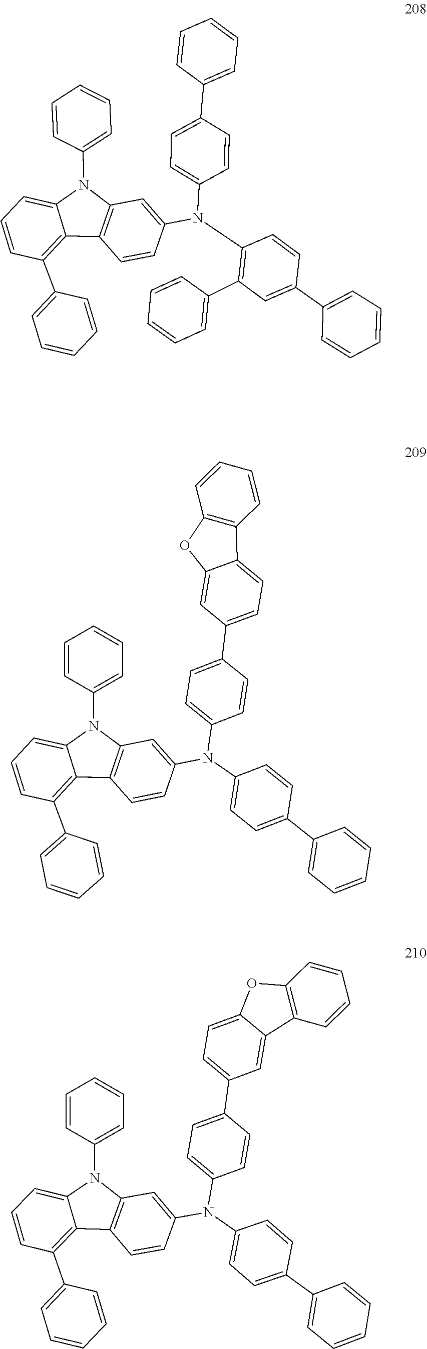 [SMILES: c1ccc(-c2ccc(N(c3ccc4c5c(-c6ccccc6)cccc5n(-c5ccccc5)c4c3)c3ccc(-c4ccccc4)cc3-c3ccccc3)cc2)cc1 ; c1ccc(-c2ccc(N(c3ccc(-c4ccc5c(c4)oc4ccccc45)cc3)c3ccc4c5c(-c6ccccc6)cccc5n(-c5ccccc5)c4c3)cc2)cc1 ; c1ccc(-c2ccc(N(c3ccc(-c4ccc5oc6ccccc6c5c4)cc3)c3ccc4c5c(-c6ccccc6)cccc5n(-c5ccccc5)c4c3)cc2)cc1]

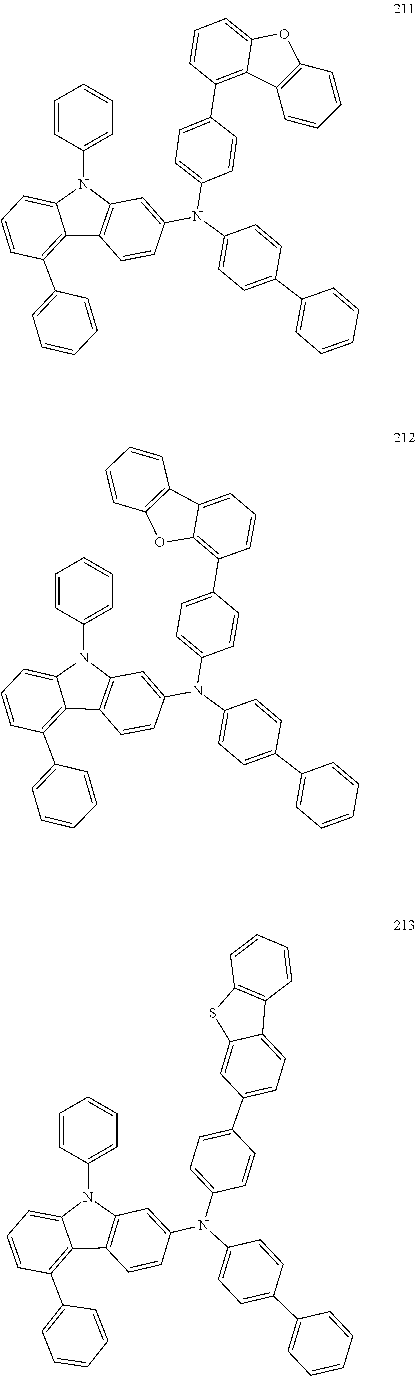 [SMILES: c1ccc(-c2ccc(N(c3ccc(-c4cccc5oc6ccccc6c45)cc3)c3ccc4c5c(-c6ccccc6)cccc5n(-c5ccccc5)c4c3)cc2)cc1 ; c1ccc(-c2ccc(N(c3ccc(-c4cccc5c4oc4ccccc45)cc3)c3ccc4c5c(-c6ccccc6)cccc5n(-c5ccccc5)c4c3)cc2)cc1 ; c1ccc(-c2ccc(N(c3ccc(-c4ccc5c(c4)sc4ccccc45)cc3)c3ccc4c5c(-c6ccccc6)cccc5n(-c5ccccc5)c4c3)cc2)cc1]

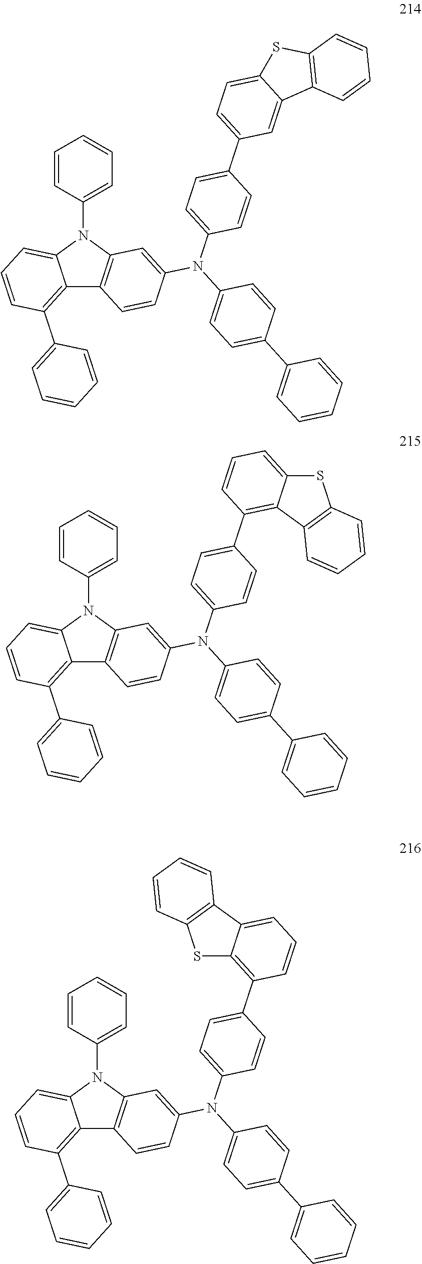

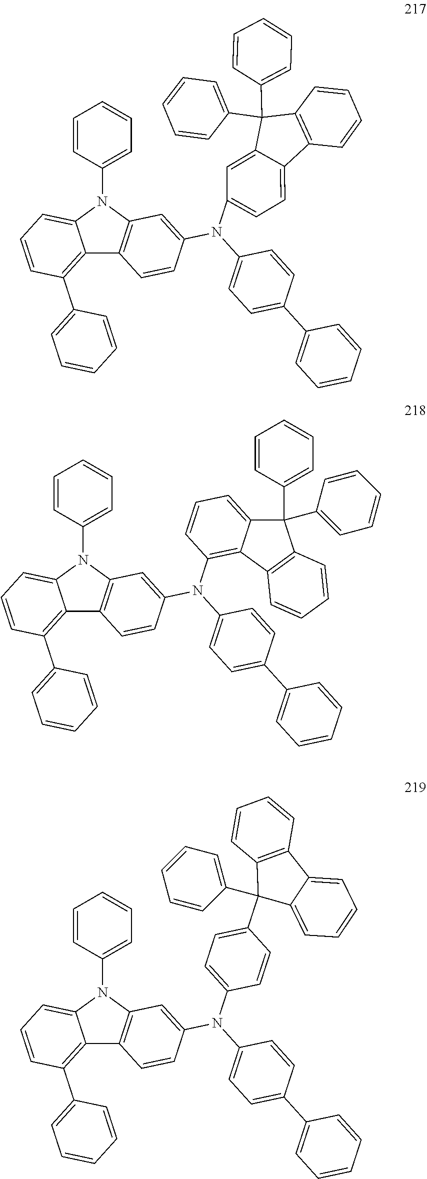 [SMILES: c1ccc(-c2ccc(N(c3ccc4c(c3)C(c3ccccc3)(c3ccccc3)c3ccccc3-4)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3cccc4c3-c3ccccc3C4(c3ccccc3)c3ccccc3)cc2)cc1 ; c1ccc(-c2ccc(N(c3ccc(C4(c5ccccc5)c5ccccc5-c5ccccc54)cc3)c3ccc4c5c(-c6ccccc6)cccc5n(-c5ccccc5)c4c3)cc2)cc1]

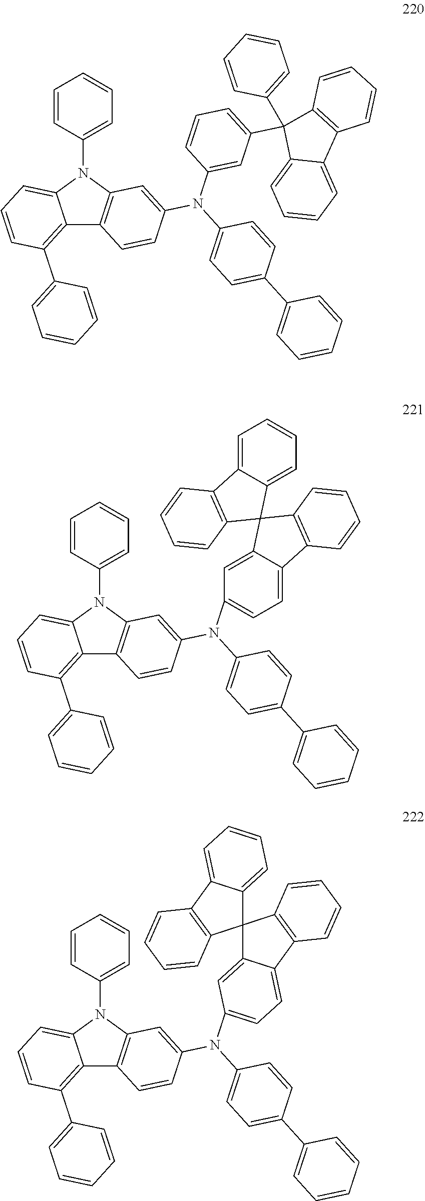 [SMILES: c1ccc(-c2ccc(N(c3cccc(C4(c5ccccc5)c5ccccc5-c5ccccc54)c3)c3ccc4c5c(-c6ccccc6)cccc5n(-c5ccccc5)c4c3)cc2)cc1 ; c1ccc(-c2ccc(N(c3ccc4c(c3)C3(c5ccccc5-c5ccccc53)c3ccccc3-4)c3ccc4c5c(-c6ccccc6)cccc5n(-c5ccccc5)c4c3)cc2)cc1 ; c1ccc(-c2ccc(N(c3ccc4c(c3)C3(c5ccccc5-c5ccccc53)c3ccccc3-4)c3ccc4c5c(-c6ccccc6)cccc5n(-c5ccccc5)c4c3)cc2)cc1]

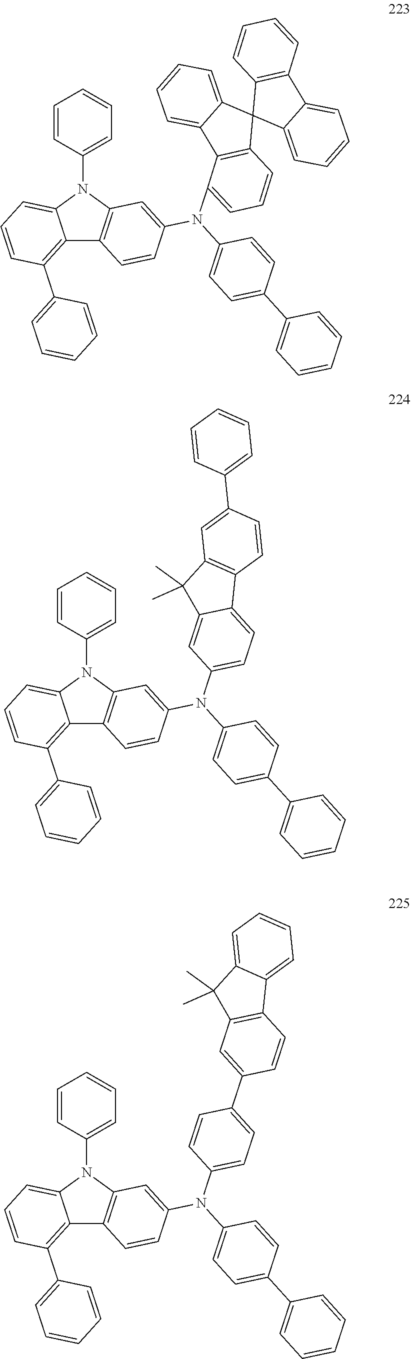 [SMILES: c1ccc(-c2ccc(N(c3ccc4c5c(-c6ccccc6)cccc5n(-c5ccccc5)c4c3)c3cccc4c3-c3ccccc3C43c4ccccc4-c4ccccc43)cc2)cc1 ; CC1(C)c2cc(-c3ccccc3)ccc2-c2ccc(N(c3ccc(-c4ccccc4)cc3)c3ccc4c5c(-c6ccccc6)cccc5n(-c5ccccc5)c4c3)cc21 ; CC1(C)c2ccccc2-c2ccc(-c3ccc(N(c4ccc(-c5ccccc5)cc4)c4ccc5c6c(-c7ccccc7)cccc6n(-c6ccccc6)c5c4)cc3)cc21]

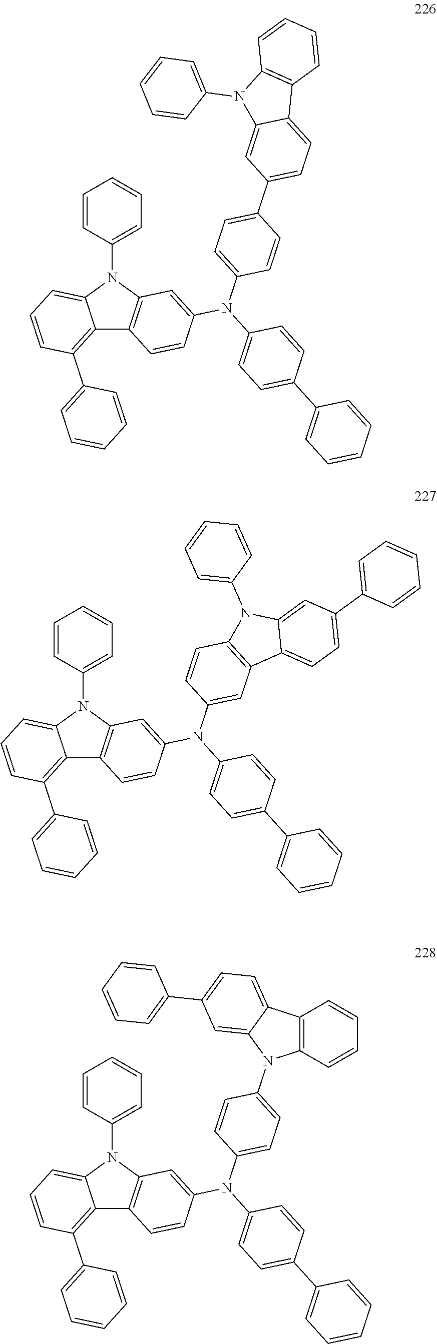 [SMILES: c1ccc(-c2ccc(N(c3ccc(-c4ccc5c6ccccc6n(-c6ccccc6)c5c4)cc3)c3ccc4c5c(-c6ccccc6)cccc5n(-c5ccccc5)c4c3)cc2)cc1 ; c1ccc(-c2ccc(N(c3ccc4c(c3)c3ccc(-c5ccccc5)cc3n4-c3ccccc3)c3ccc4c5c(-c6ccccc6)cccc5n(-c5ccccc5)c4c3)cc2)cc1 ; c1ccc(-c2ccc(N(c3ccc(-n4c5ccccc5c5ccc(-c6ccccc6)cc54)cc3)c3ccc4c5c(-c6ccccc6)cccc5n(-c5ccccc5)c4c3)cc2)cc1]

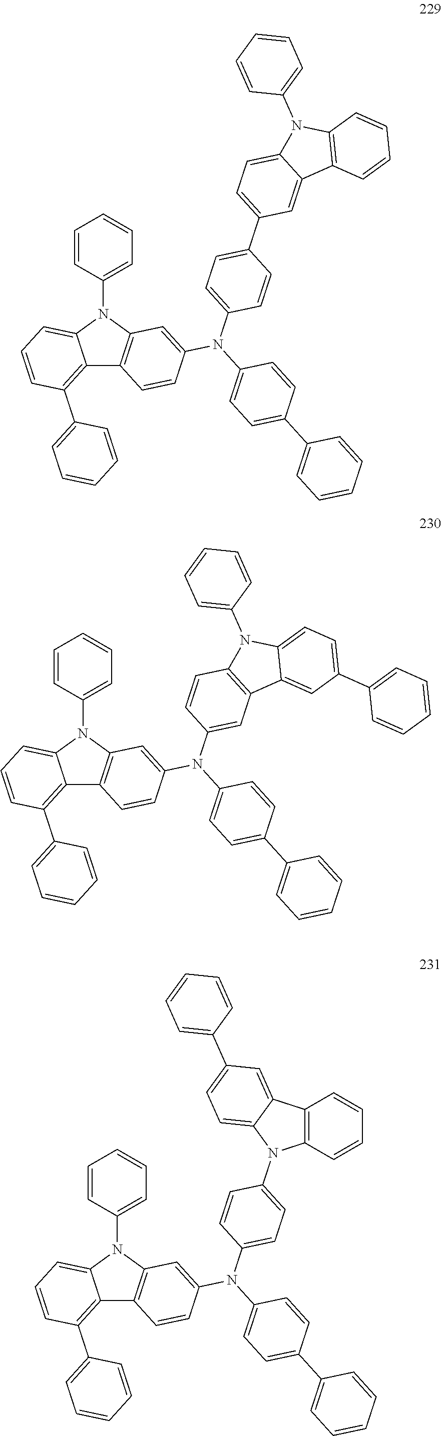 [SMILES: c1ccc(-c2ccc(N(c3ccc(-c4ccc5c(c4)c4ccccc4n5-c4ccccc4)cc3)c3ccc4c5c(-c6ccccc6)cccc5n(-c5ccccc5)c4c3)cc2)cc1 ; c1ccc(-c2ccc(N(c3ccc4c(c3)c3cc(-c5ccccc5)ccc3n4-c3ccccc3)c3ccc4c5c(-c6ccccc6)cccc5n(-c5ccccc5)c4c3)cc2)cc1 ; c1ccc(-c2ccc(N(c3ccc(-n4c5ccccc5c5cc(-c6ccccc6)ccc54)cc3)c3ccc4c5c(-c6ccccc6)cccc5n(-c5ccccc5)c4c3)cc2)cc1]

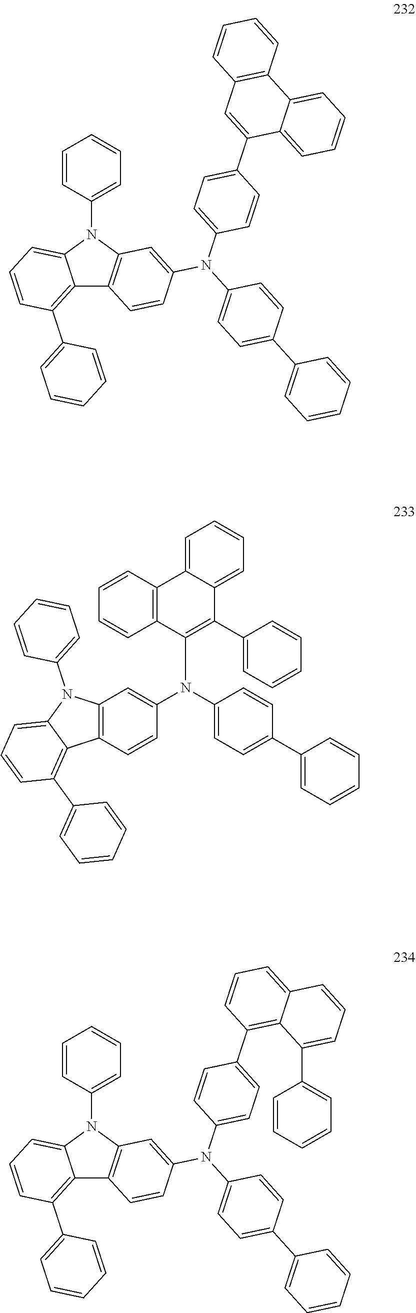 [SMILES: c1ccc(-c2ccc(N(c3ccc(-c4cc5ccccc5c5ccccc45)cc3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3c(-c4ccccc4)c4ccccc4c4ccccc34)cc2)cc1 ; c1ccc(-c2ccc(N(c3ccc(-c4cccc5cccc(-c6ccccc6)c45)cc3)c3ccc4c5c(-c6ccccc6)cccc5n(-c5ccccc5)c4c3)cc2)cc1]

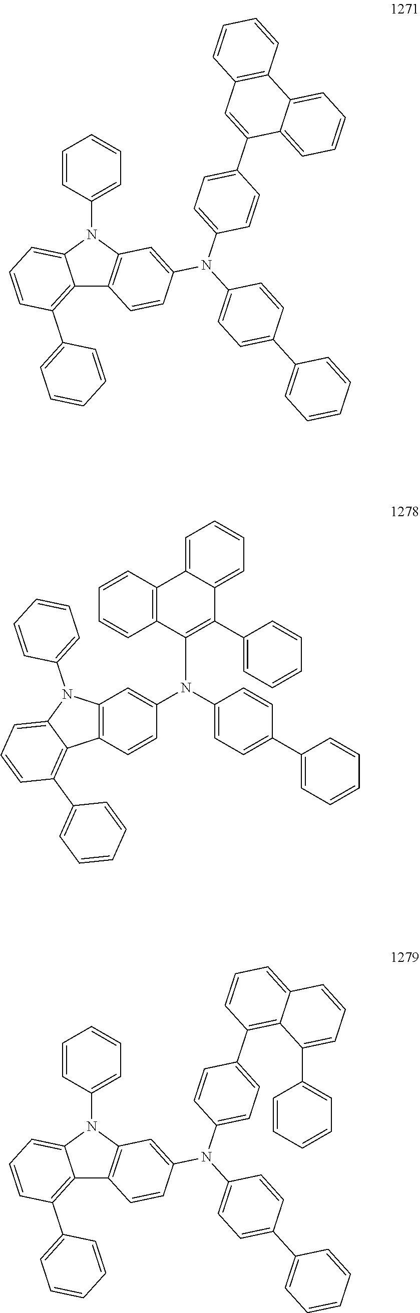 [SMILES: c1ccc(-c2ccc(N(c3ccc(-c4cc5ccccc5c5ccccc45)cc3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3c(-c4ccccc4)c4ccccc4c4ccccc34)cc2)cc1 ; c1ccc(-c2ccc(N(c3ccc(-c4cccc5cccc(-c6ccccc6)c45)cc3)c3ccc4c5c(-c6ccccc6)cccc5n(-c5ccccc5)c4c3)cc2)cc1]

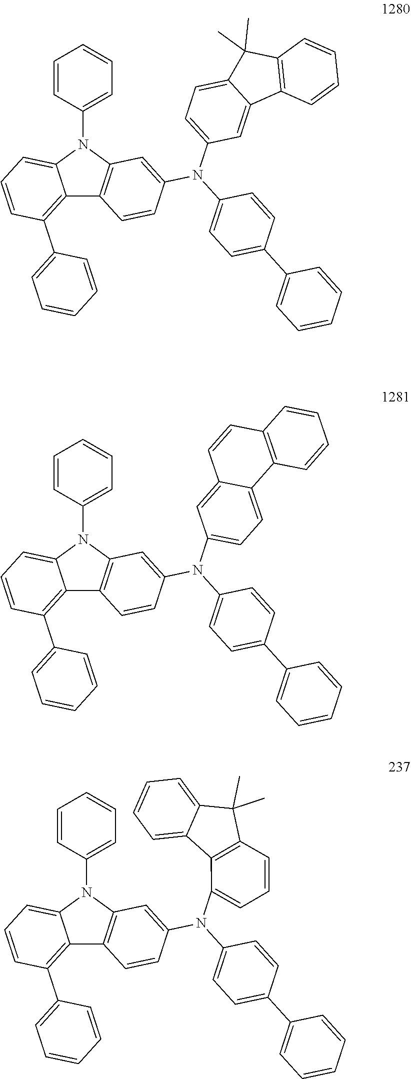 [SMILES: CC1(C)c2ccccc2-c2cc(N(c3ccc(-c4ccccc4)cc3)c3ccc4c5c(-c6ccccc6)cccc5n(-c5ccccc5)c4c3)ccc21 ; c1ccc(-c2ccc(N(c3ccc4c(ccc5ccccc54)c3)c3ccc4c5c(-c6ccccc6)cccc5n(-c5ccccc5)c4c3)cc2)cc1 ; CC1(C)c2ccccc2-c2c(N(c3ccc(-c4ccccc4)cc3)c3ccc4c5c(-c6ccccc6)cccc5n(-c5ccccc5)c4c3)cccc21]

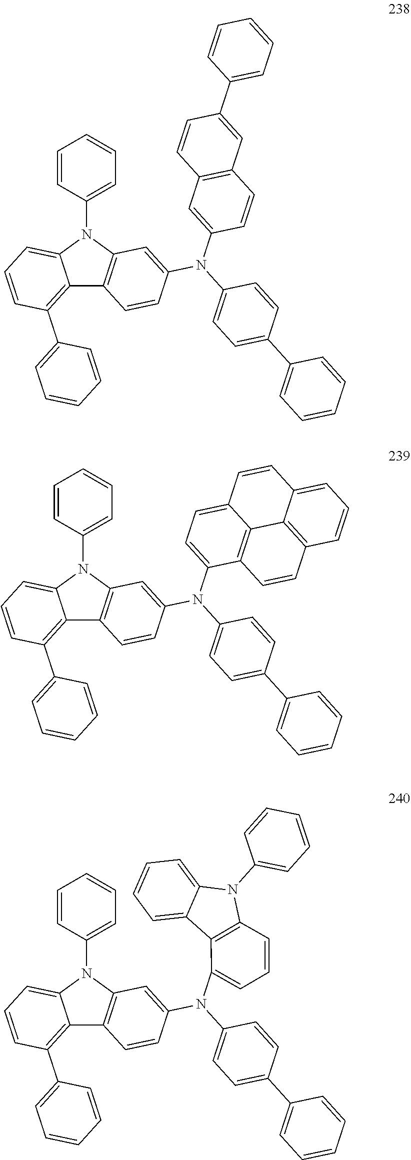 [SMILES: c1ccc(-c2ccc(N(c3ccc4cc(-c5ccccc5)ccc4c3)c3ccc4c5c(-c6ccccc6)cccc5n(-c5ccccc5)c4c3)cc2)cc1 ; c1ccc(-c2ccc(N(c3ccc4c5c(-c6ccccc6)cccc5n(-c5ccccc5)c4c3)c3ccc4ccc5cccc6ccc3c4c56)cc2)cc1 ; c1ccc(-c2ccc(N(c3ccc4c5c(-c6ccccc6)cccc5n(-c5ccccc5)c4c3)c3cccc4c3c3ccccc3n4-c3ccccc3)cc2)cc1]

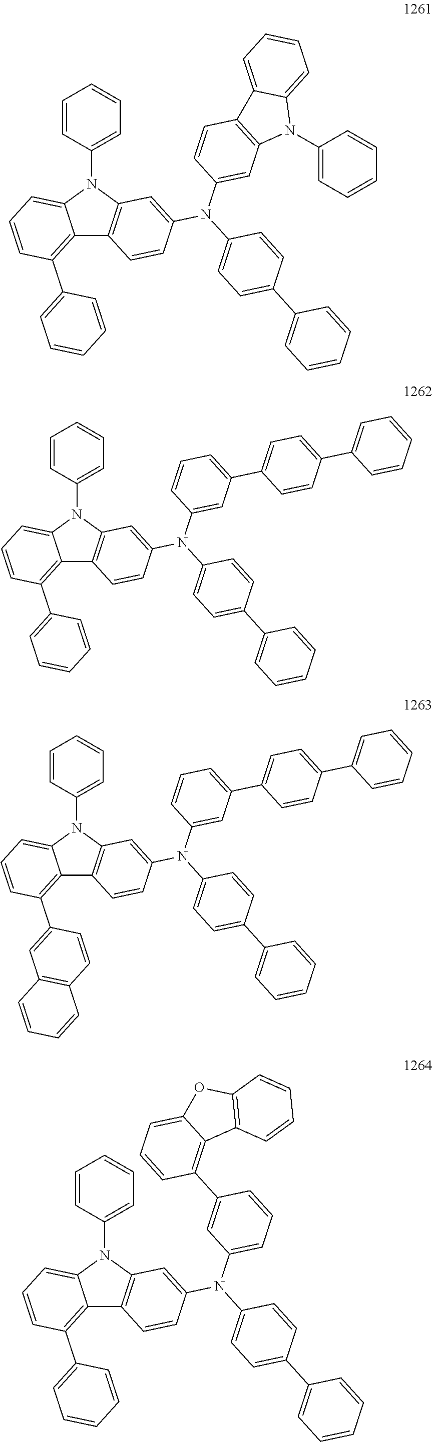 [SMILES: c1ccc(-c2ccc(N(c3ccc4c5ccccc5n(-c5ccccc5)c4c3)c3ccc4c5c(-c6ccccc6)cccc5n(-c5ccccc5)c4c3)cc2)cc1 ; c1ccc(-c2ccc(-c3cccc(N(c4ccc(-c5ccccc5)cc4)c4ccc5c6c(-c7ccccc7)cccc6n(-c6ccccc6)c5c4)c3)cc2)cc1 ; c1ccc(-c2ccc(-c3cccc(N(c4ccc(-c5ccccc5)cc4)c4ccc5c6c(-c7ccc8ccccc8c7)cccc6n(-c6ccccc6)c5c4)c3)cc2)cc1 ; c1ccc(-c2ccc(N(c3cccc(-c4cccc5oc6ccccc6c45)c3)c3ccc4c5c(-c6ccccc6)cccc5n(-c5ccccc5)c4c3)cc2)cc1]

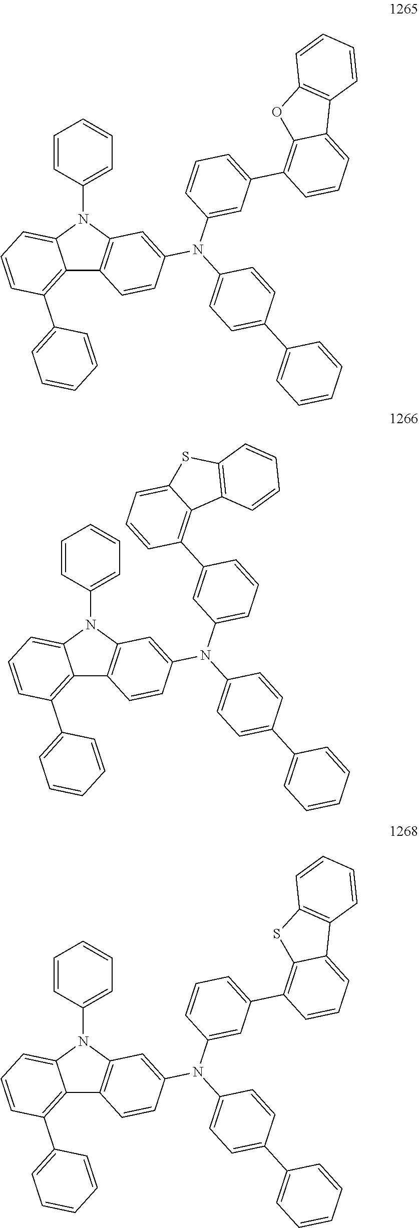 [SMILES: c1ccc(-c2ccc(N(c3cccc(-c4cccc5c4oc4ccccc45)c3)c3ccc4c5c(-c6ccccc6)cccc5n(-c5ccccc5)c4c3)cc2)cc1 ; c1ccc(-c2ccc(N(c3cccc(-c4cccc5sc6ccccc6c45)c3)c3ccc4c5c(-c6ccccc6)cccc5n(-c5ccccc5)c4c3)cc2)cc1 ; c1ccc(-c2ccc(N(c3cccc(-c4cccc5c4sc4ccccc45)c3)c3ccc4c5c(-c6ccccc6)cccc5n(-c5ccccc5)c4c3)cc2)cc1]

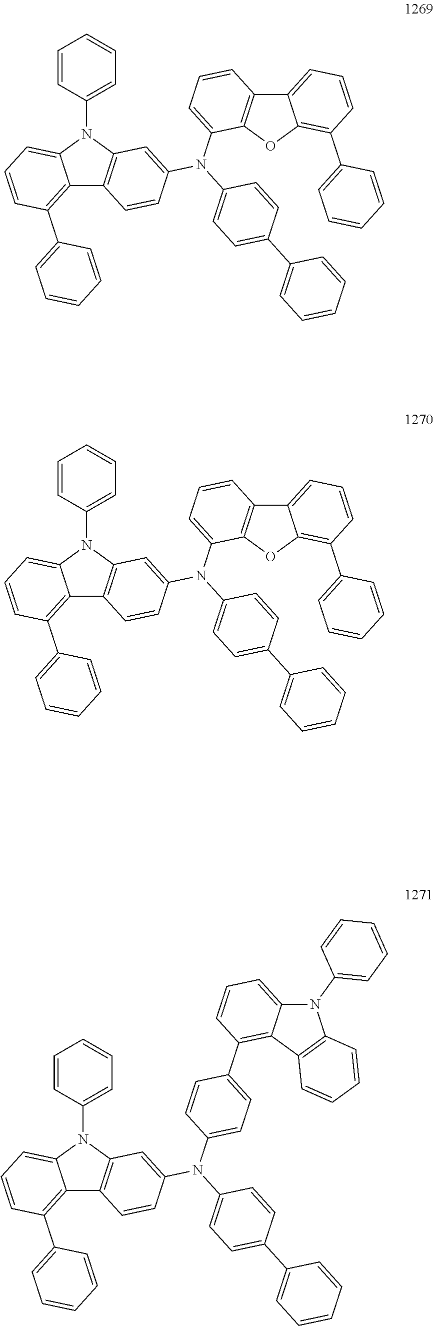 [SMILES: c1ccc(-c2ccc(N(c3ccc4c5c(-c6ccccc6)cccc5n(-c5ccccc5)c4c3)c3cccc4c3oc3c(-c5ccccc5)cccc34)cc2)cc1 ; c1ccc(-c2ccc(N(c3ccc4c5c(-c6ccccc6)cccc5n(-c5ccccc5)c4c3)c3cccc4c3oc3c(-c5ccccc5)cccc34)cc2)cc1 ; c1ccc(-c2ccc(N(c3ccc(-c4cccc5c4c4ccccc4n5-c4ccccc4)cc3)c3ccc4c5c(-c6ccccc6)cccc5n(-c5ccccc5)c4c3)cc2)cc1]

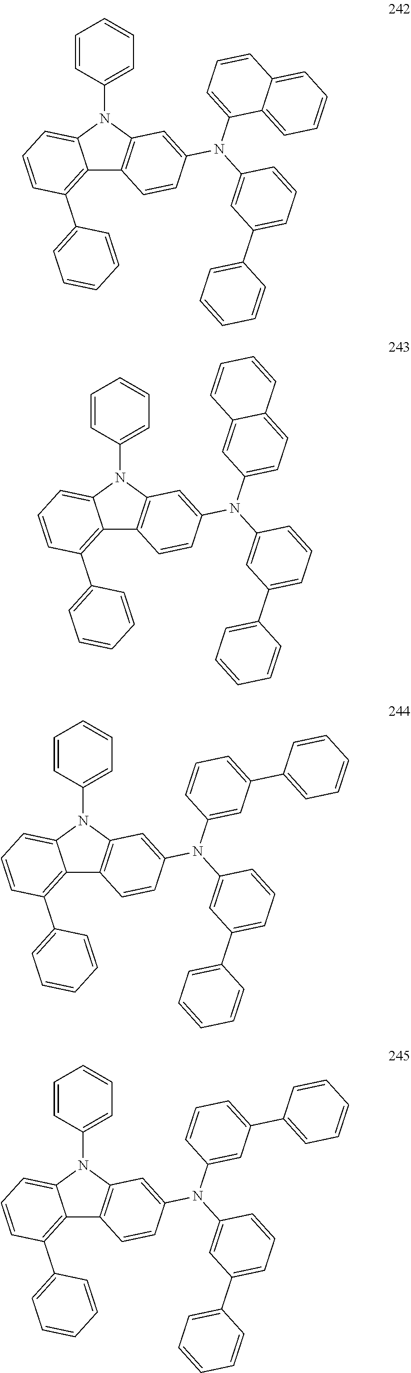

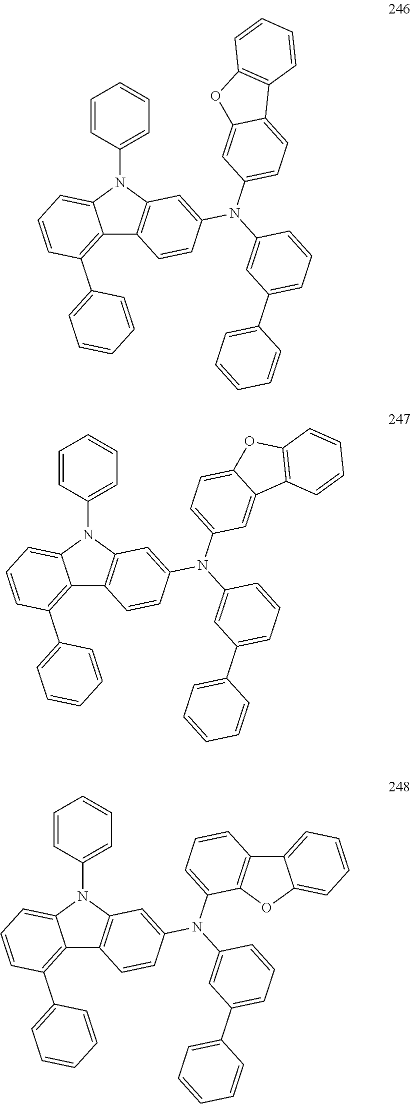 [SMILES: c1ccc(-c2cccc(N(c3ccc4c(c3)oc3ccccc34)c3ccc4c5c(-c6ccccc6)cccc5n(-c5ccccc5)c4c3)c2)cc1 ; c1ccc(-c2cccc(N(c3ccc4oc5ccccc5c4c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3oc3ccccc34)c2)cc1]

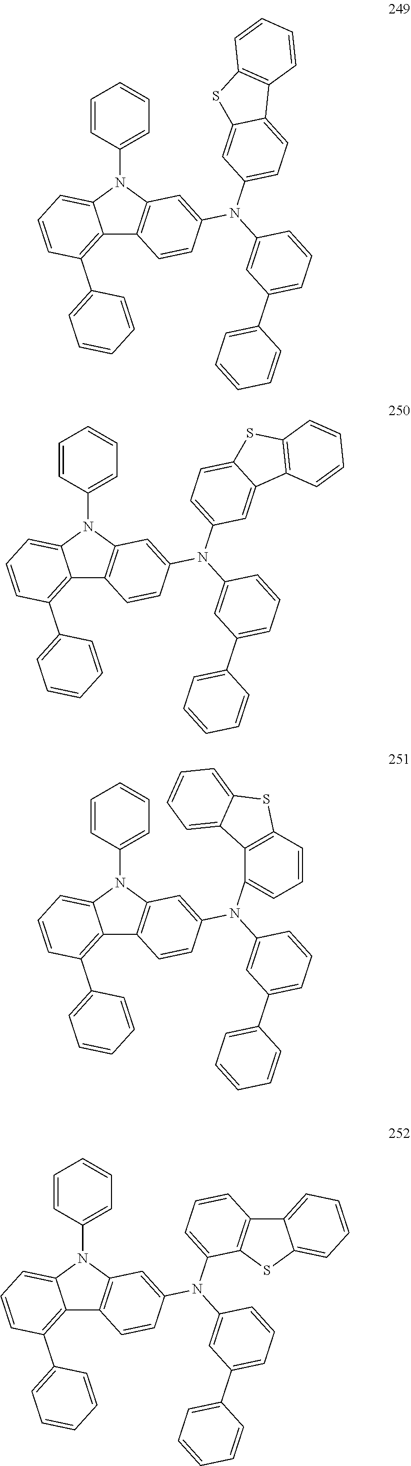

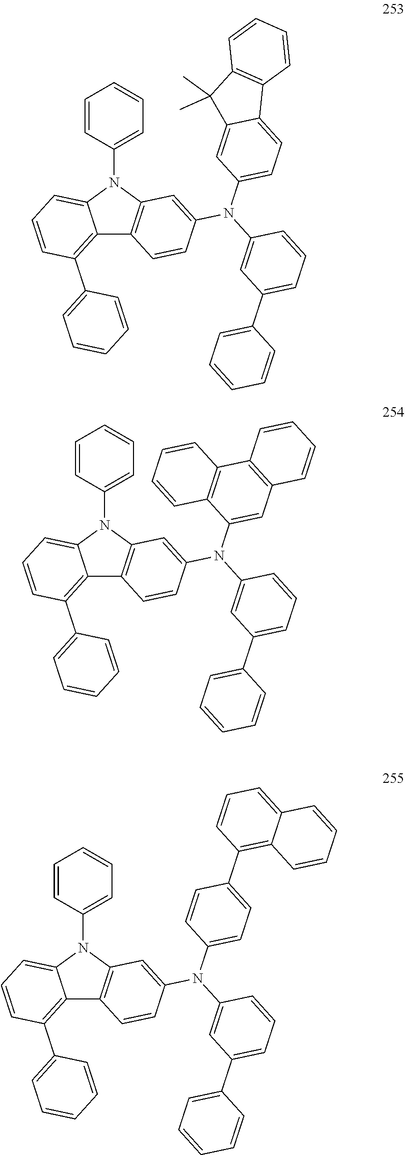

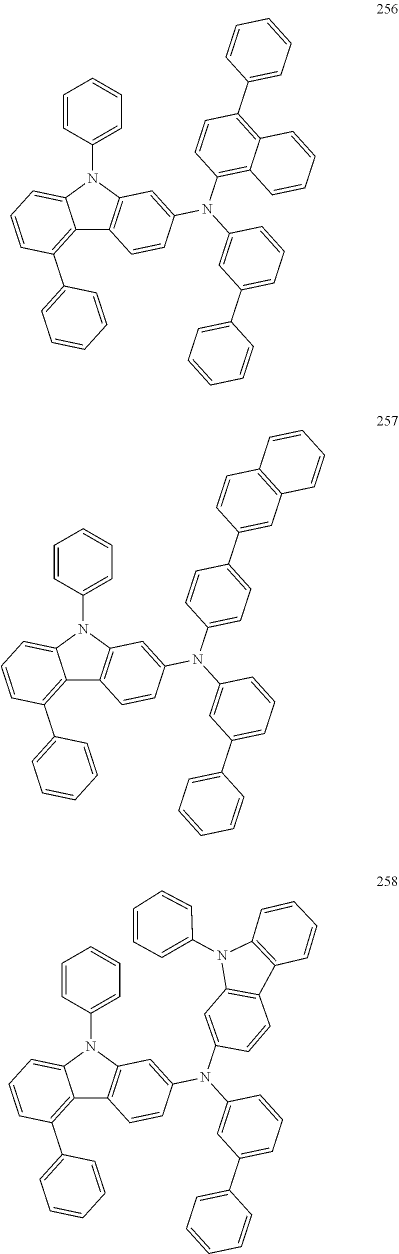 [SMILES: c1ccc(-c2cccc(N(c3ccc4c5c(-c6ccccc6)cccc5n(-c5ccccc5)c4c3)c3ccc(-c4ccccc4)c4ccccc34)c2)cc1 ; c1ccc(-c2cccc(N(c3ccc(-c4ccc5ccccc5c4)cc3)c3ccc4c5c(-c6ccccc6)cccc5n(-c5ccccc5)c4c3)c2)cc1 ; c1ccc(-c2cccc(N(c3ccc4c5ccccc5n(-c5ccccc5)c4c3)c3ccc4c5c(-c6ccccc6)cccc5n(-c5ccccc5)c4c3)c2)cc1]

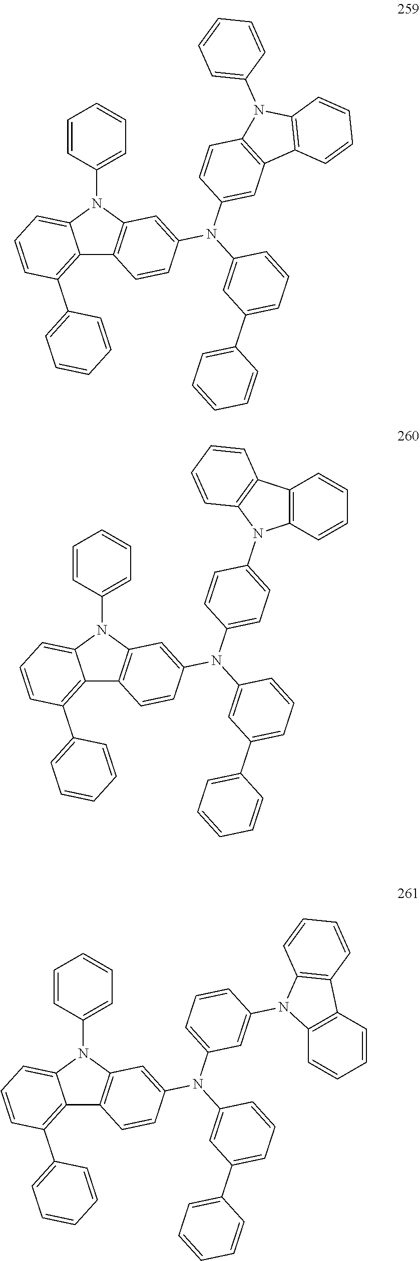

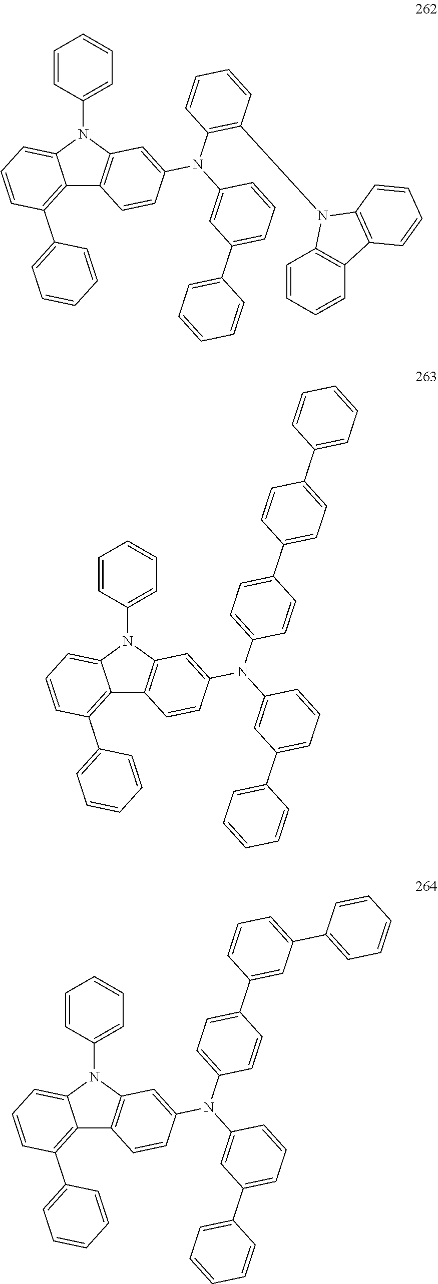 [SMILES: c1ccc(-c2cccc(N(c3ccc4c5c(-c6ccccc6)cccc5n(-c5ccccc5)c4c3)c3ccccc3-n3c4ccccc4c4ccccc43)c2)cc1 ; c1ccc(-c2ccc(-c3ccc(N(c4cccc(-c5ccccc5)c4)c4ccc5c6c(-c7ccccc7)cccc6n(-c6ccccc6)c5c4)cc3)cc2)cc1 ; c1ccc(-c2cccc(-c3ccc(N(c4cccc(-c5ccccc5)c4)c4ccc5c6c(-c7ccccc7)cccc6n(-c6ccccc6)c5c4)cc3)c2)cc1]

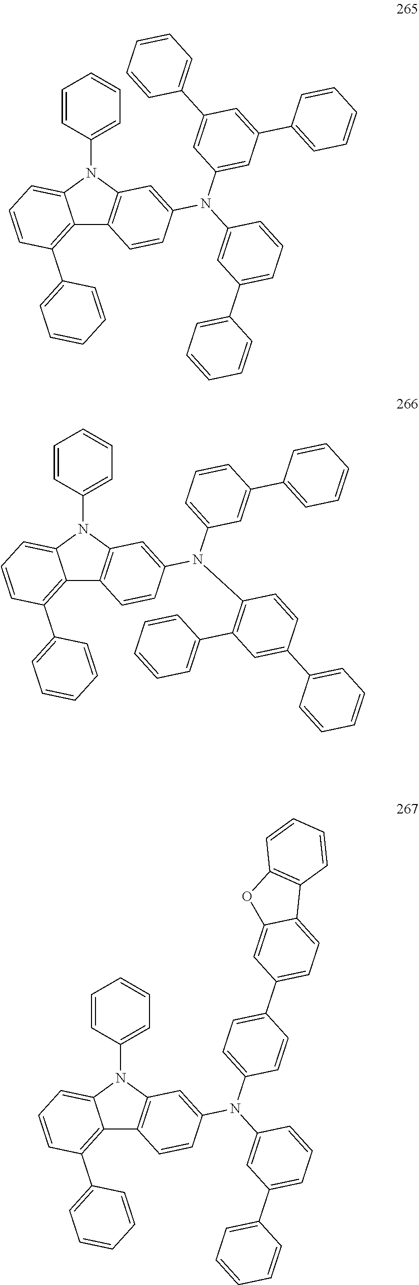 [SMILES: c1ccc(-c2cccc(N(c3cc(-c4ccccc4)cc(-c4ccccc4)c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3ccc(-c4ccccc4)cc3-c3ccccc3)c2)cc1 ; c1ccc(-c2cccc(N(c3ccc(-c4ccc5c(c4)oc4ccccc45)cc3)c3ccc4c5c(-c6ccccc6)cccc5n(-c5ccccc5)c4c3)c2)cc1]

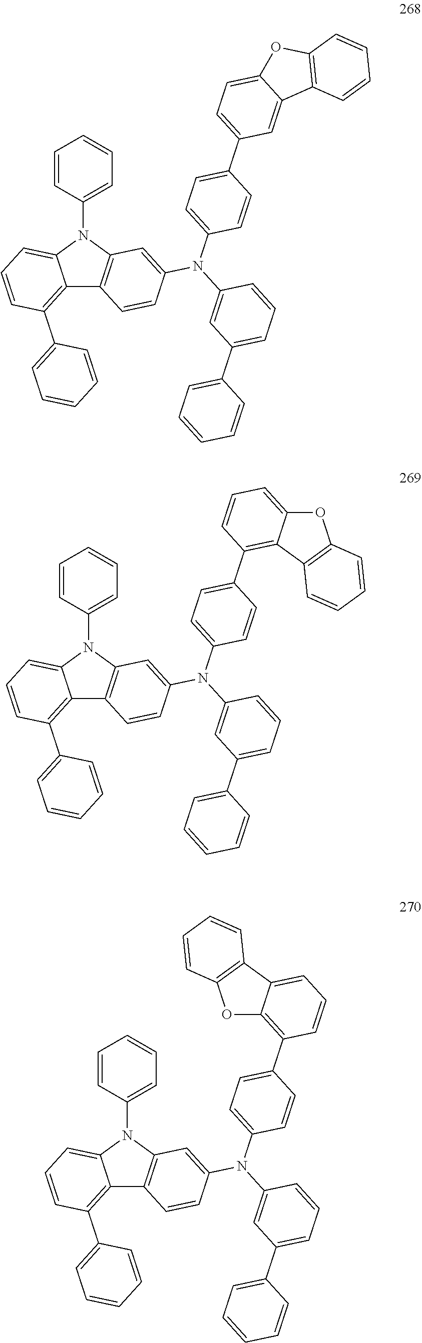 [SMILES: c1ccc(-c2cccc(N(c3ccc(-c4ccc5oc6ccccc6c5c4)cc3)c3ccc4c5c(-c6ccccc6)cccc5n(-c5ccccc5)c4c3)c2)cc1 ; c1ccc(-c2cccc(N(c3ccc(-c4cccc5oc6ccccc6c45)cc3)c3ccc4c5c(-c6ccccc6)cccc5n(-c5ccccc5)c4c3)c2)cc1 ; c1ccc(-c2cccc(N(c3ccc(-c4cccc5c4oc4ccccc45)cc3)c3ccc4c5c(-c6ccccc6)cccc5n(-c5ccccc5)c4c3)c2)cc1]

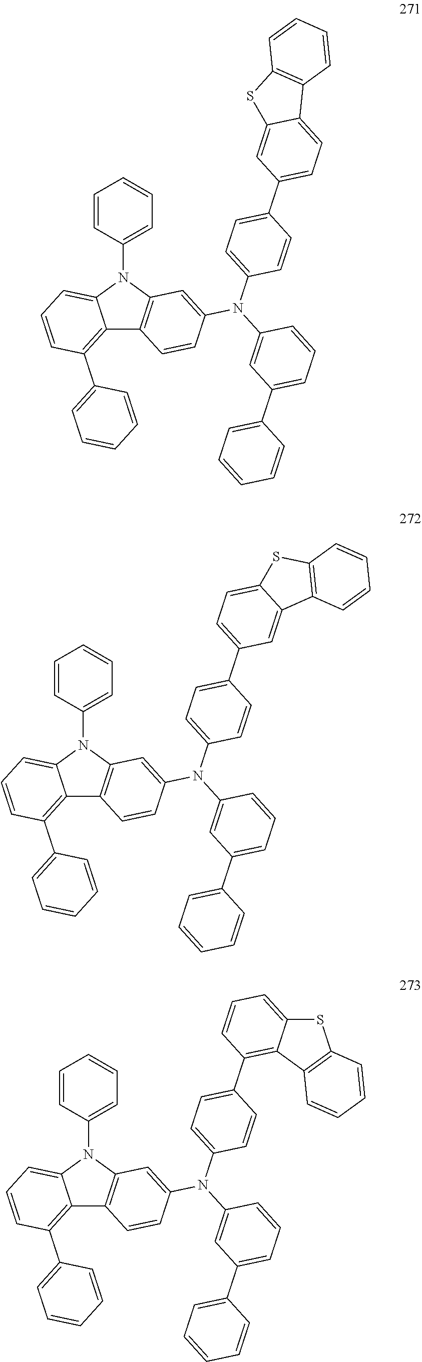 [SMILES: c1ccc(-c2cccc(N(c3ccc(-c4ccc5c(c4)sc4ccccc45)cc3)c3ccc4c5c(-c6ccccc6)cccc5n(-c5ccccc5)c4c3)c2)cc1 ; c1ccc(-c2cccc(N(c3ccc(-c4ccc5sc6ccccc6c5c4)cc3)c3ccc4c5c(-c6ccccc6)cccc5n(-c5ccccc5)c4c3)c2)cc1 ; c1ccc(-c2cccc(N(c3ccc(-c4cccc5sc6ccccc6c45)cc3)c3ccc4c5c(-c6ccccc6)cccc5n(-c5ccccc5)c4c3)c2)cc1]

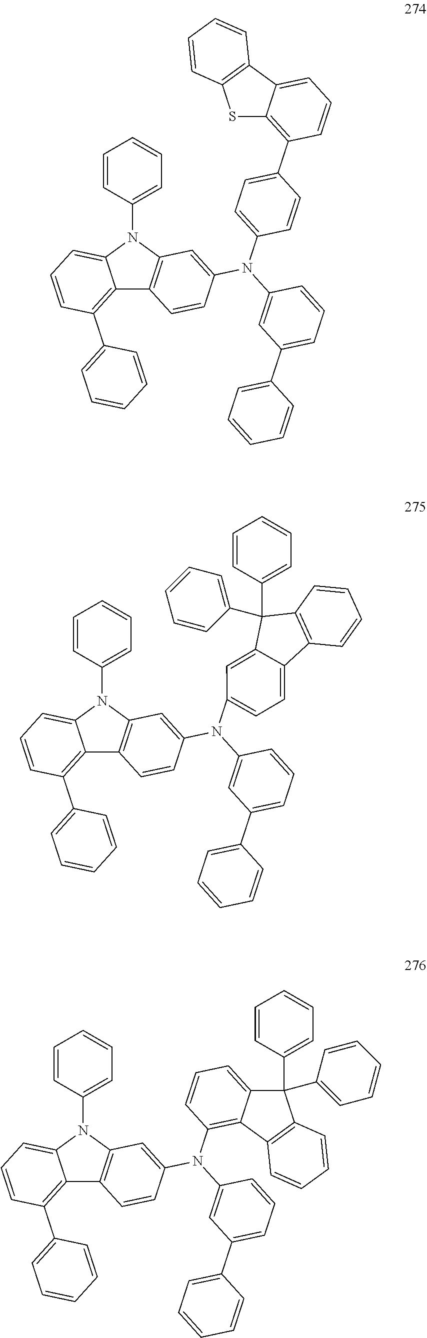 [SMILES: c1ccc(-c2cccc(N(c3ccc(-c4cccc5c4sc4ccccc45)cc3)c3ccc4c5c(-c6ccccc6)cccc5n(-c5ccccc5)c4c3)c2)cc1 ; c1ccc(-c2cccc(N(c3ccc4c(c3)C(c3ccccc3)(c3ccccc3)c3ccccc3-4)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3-c3ccccc3C4(c3ccccc3)c3ccccc3)c2)cc1]

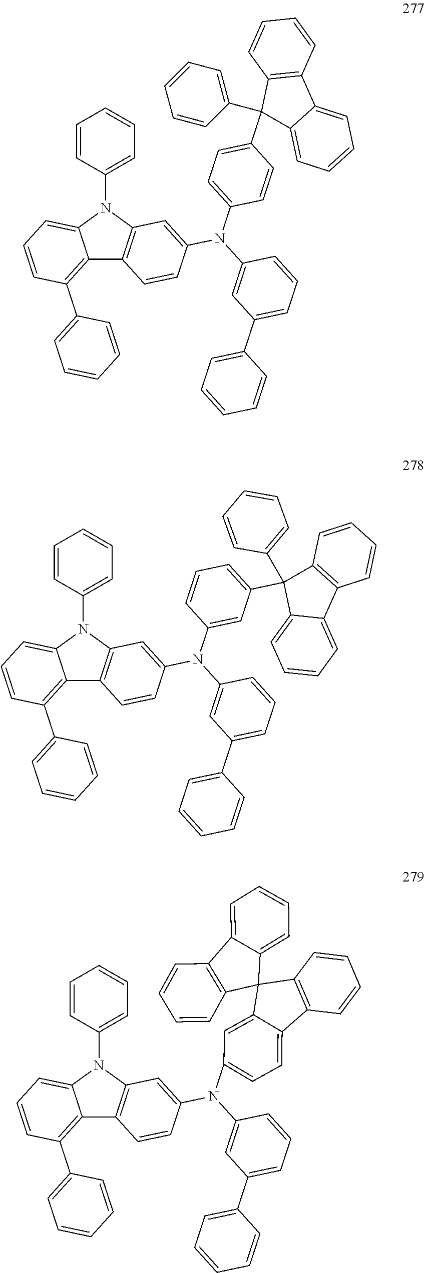 [SMILES: c1ccc(-c2cccc(N(c3ccc(C4(c5ccccc5)c5ccccc5-c5ccccc54)cc3)c3ccc4c5c(-c6ccccc6)cccc5n(-c5ccccc5)c4c3)c2)cc1 ; c1ccc(-c2cccc(N(c3cccc(C4(c5ccccc5)c5ccccc5-c5ccccc54)c3)c3ccc4c5c(-c6ccccc6)cccc5n(-c5ccccc5)c4c3)c2)cc1 ; c1ccc(-c2cccc(N(c3ccc4c(c3)C3(c5ccccc5-c5ccccc53)c3ccccc3-4)c3ccc4c5c(-c6ccccc6)cccc5n(-c5ccccc5)c4c3)c2)cc1]

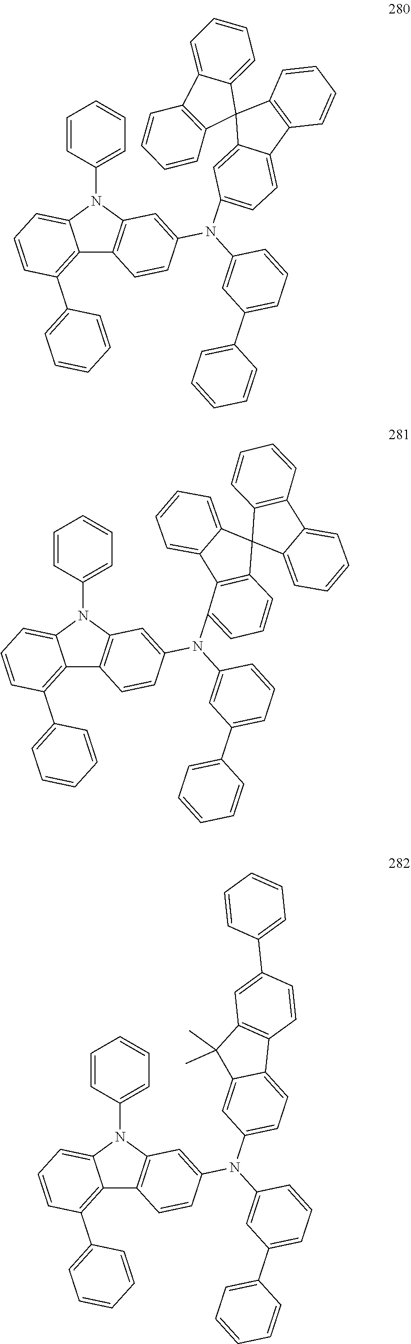 [SMILES: c1ccc(-c2cccc(N(c3ccc4c(c3)C3(c5ccccc5-c5ccccc53)c3ccccc3-4)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3-c3ccccc3C43c4ccccc4-c4ccccc43)c2)cc1 ; CC1(C)c2cc(-c3ccccc3)ccc2-c2ccc(N(c3cccc(-c4ccccc4)c3)c3ccc4c5c(-c6ccccc6)cccc5n(-c5ccccc5)c4c3)cc21]

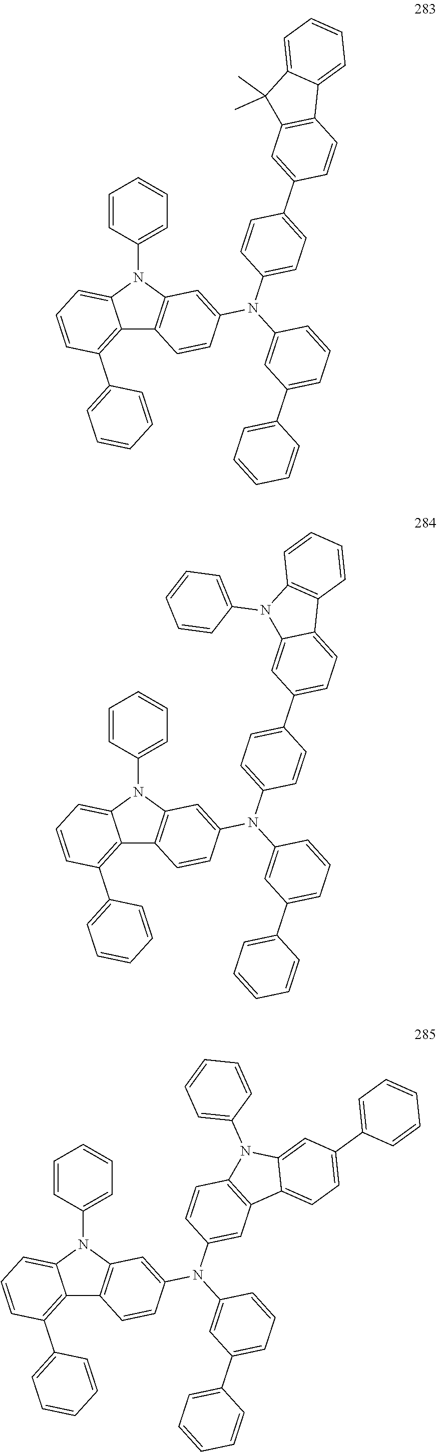 [SMILES: CC1(C)c2ccccc2-c2ccc(-c3ccc(N(c4cccc(-c5ccccc5)c4)c4ccc5c6c(-c7ccccc7)cccc6n(-c6ccccc6)c5c4)cc3)cc21 ; c1ccc(-c2cccc(N(c3ccc(-c4ccc5c6ccccc6n(-c6ccccc6)c5c4)cc3)c3ccc4c5c(-c6ccccc6)cccc5n(-c5ccccc5)c4c3)c2)cc1 ; c1ccc(-c2cccc(N(c3ccc4c(c3)c3ccc(-c5ccccc5)cc3n4-c3ccccc3)c3ccc4c5c(-c6ccccc6)cccc5n(-c5ccccc5)c4c3)c2)cc1]

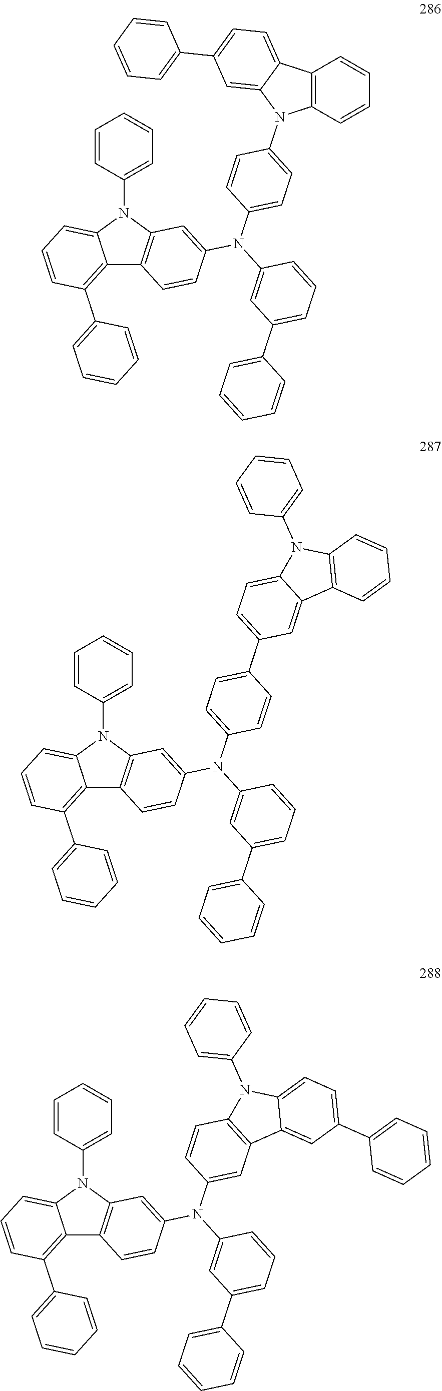

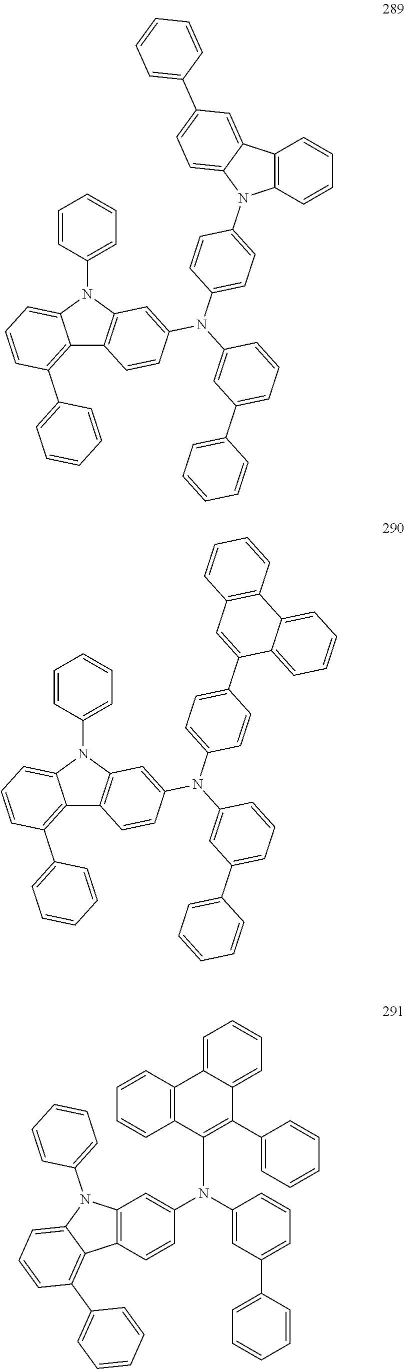

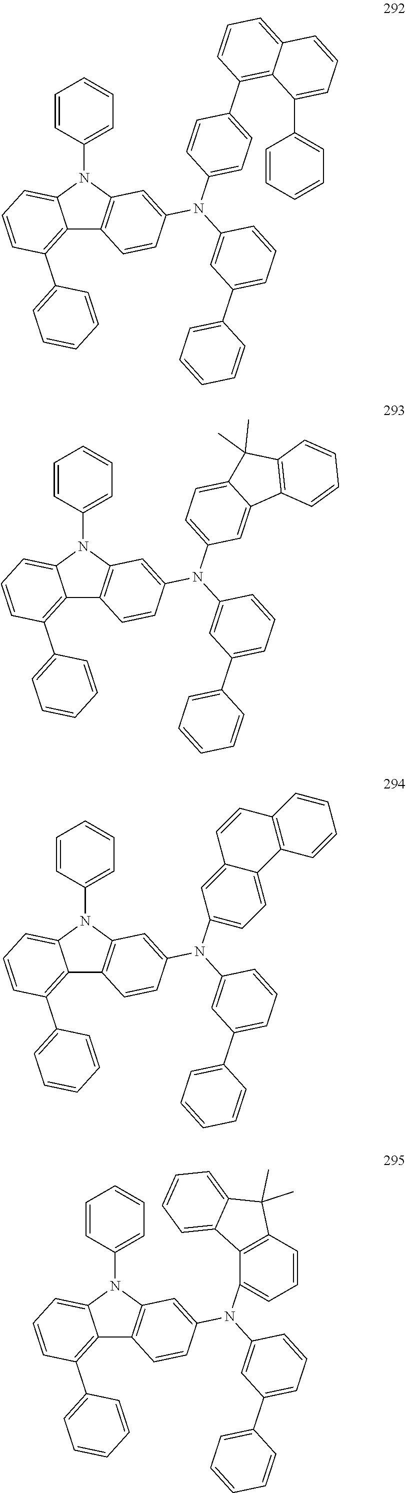

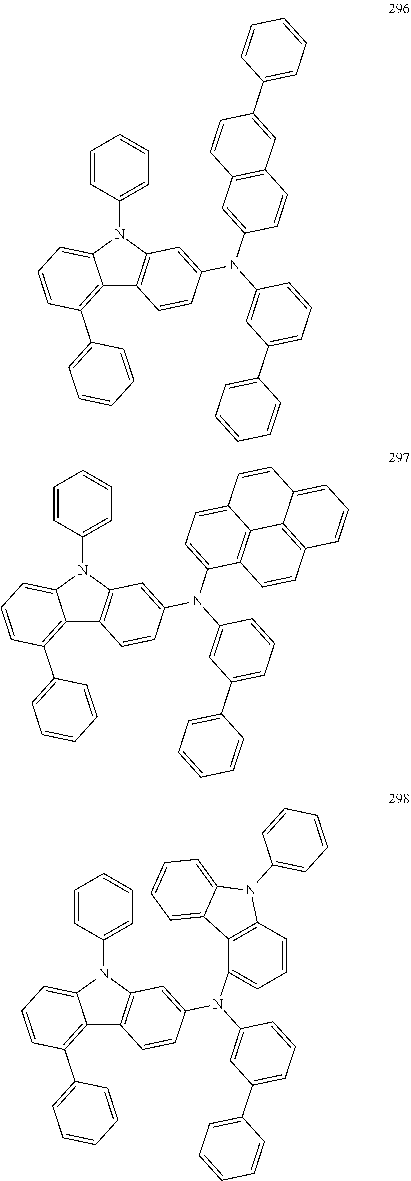

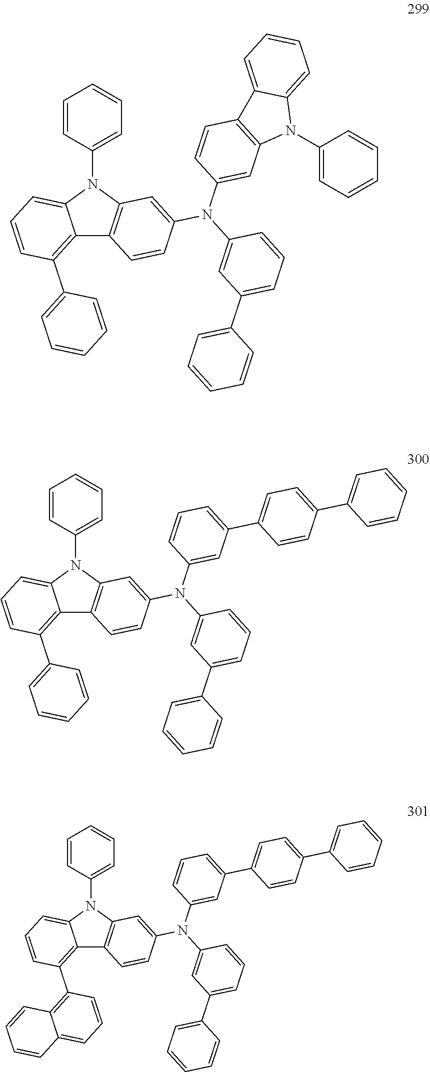 [SMILES: c1ccc(-c2cccc(N(c3ccc4c5ccccc5n(-c5ccccc5)c4c3)c3ccc4c5c(-c6ccccc6)cccc5n(-c5ccccc5)c4c3)c2)cc1 ; c1ccc(-c2ccc(-c3cccc(N(c4cccc(-c5ccccc5)c4)c4ccc5c6c(-c7ccccc7)cccc6n(-c6ccccc6)c5c4)c3)cc2)cc1 ; c1ccc(-c2ccc(-c3cccc(N(c4cccc(-c5ccccc5)c4)c4ccc5c6c(-c7cccc8ccccc78)cccc6n(-c6ccccc6)c5c4)c3)cc2)cc1]

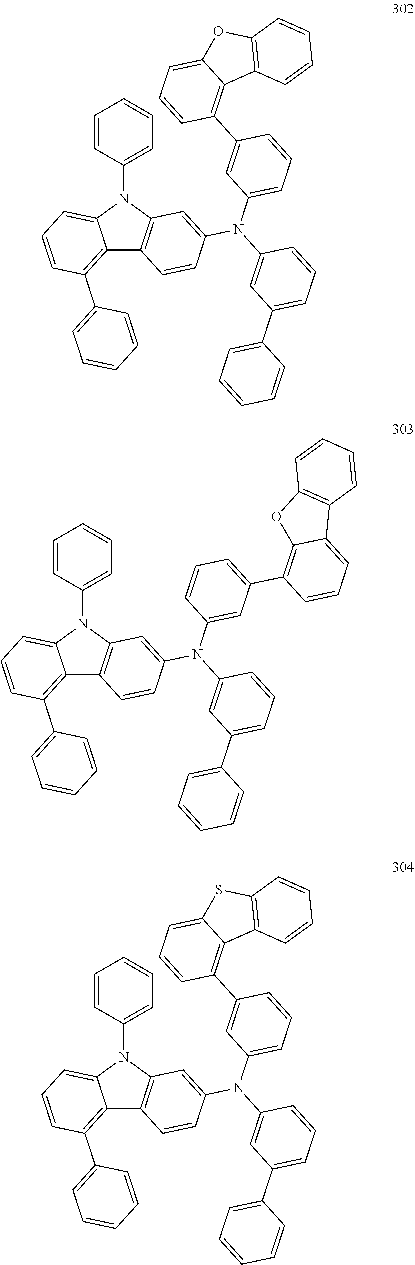 [SMILES: c1ccc(-c2cccc(N(c3cccc(-c4cccc5oc6ccccc6c45)c3)c3ccc4c5c(-c6ccccc6)cccc5n(-c5ccccc5)c4c3)c2)cc1 ; c1ccc(-c2cccc(N(c3cccc(-c4cccc5c4oc4ccccc45)c3)c3ccc4c5c(-c6ccccc6)cccc5n(-c5ccccc5)c4c3)c2)cc1 ; c1ccc(-c2cccc(N(c3cccc(-c4cccc5sc6ccccc6c45)c3)c3ccc4c5c(-c6ccccc6)cccc5n(-c5ccccc5)c4c3)c2)cc1]

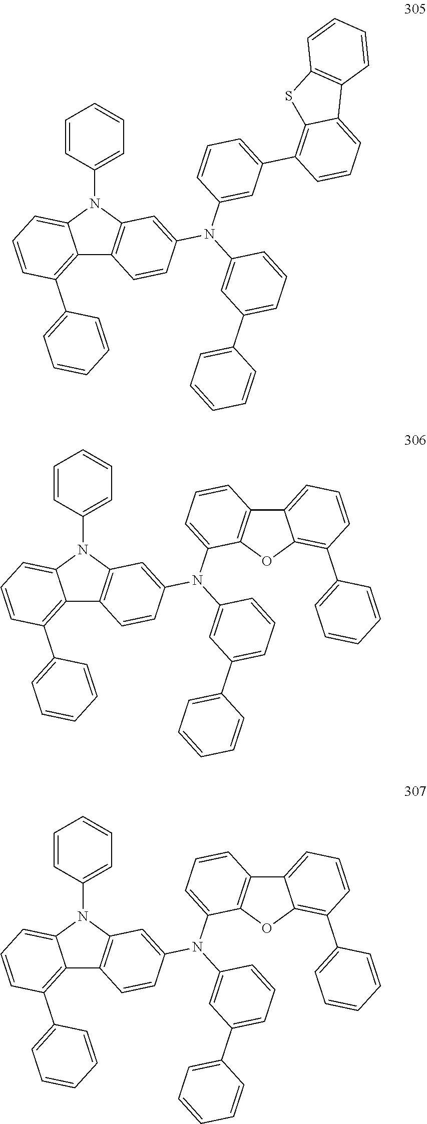 [SMILES: c1ccc(-c2cccc(N(c3cccc(-c4cccc5c4sc4ccccc45)c3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4c3oc3c(-c5ccccc5)cccc34)c2)cc1 ; c1ccc(-c2cccc(N(c3ccc4c5c(-c6ccccc6)cccc5n(-c5ccccc5)c4c3)c3cccc4c3oc3c(-c5ccccc5)cccc34)c2)cc1]

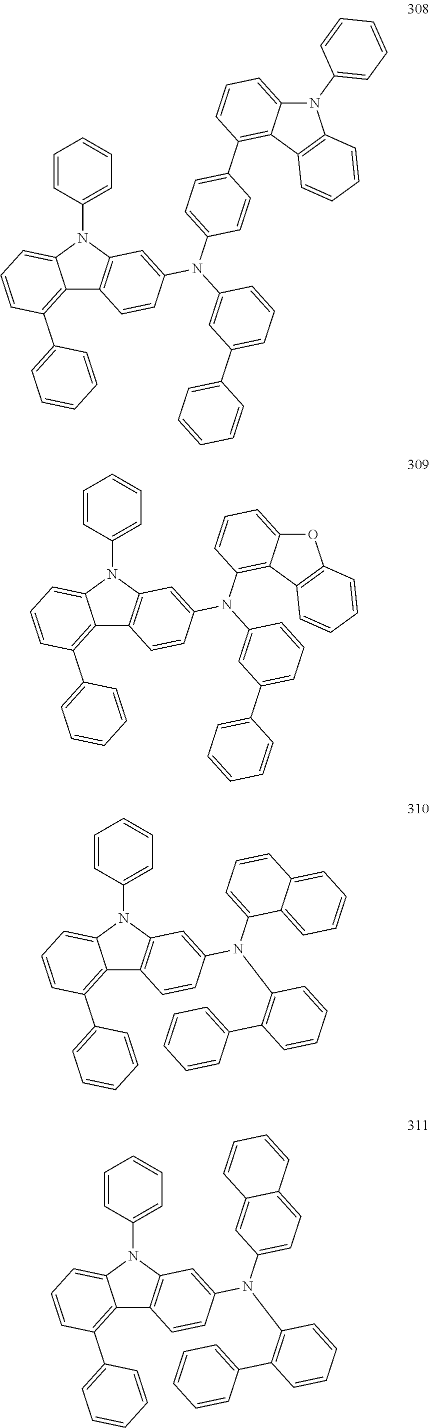 [SMILES: c1ccc(-c2cccc(N(c3ccc(-c4cccc5c4c4ccccc4n5-c4ccccc4)cc3)c3ccc4c5c(-c6ccccc6)cccc5n(-c5ccccc5)c4c3)c2)cc1 ; c1ccc(-c2cccc(N(c3ccc4c5c(-c6ccccc6)cccc5n(-c5ccccc5)c4c3)c3cccc4oc5ccccc5c34)c2)cc1 ; c1ccc(-c2ccccc2N(c2ccc3c4c(-c5ccccc5)cccc4n(-c4ccccc4)c3c2)c2cccc3ccccc23)cc1 ; c1ccc(-c2ccccc2N(c2ccc3ccccc3c2)c2ccc3c4c(-c5ccccc5)cccc4n(-c4ccccc4)c3c2)cc1]

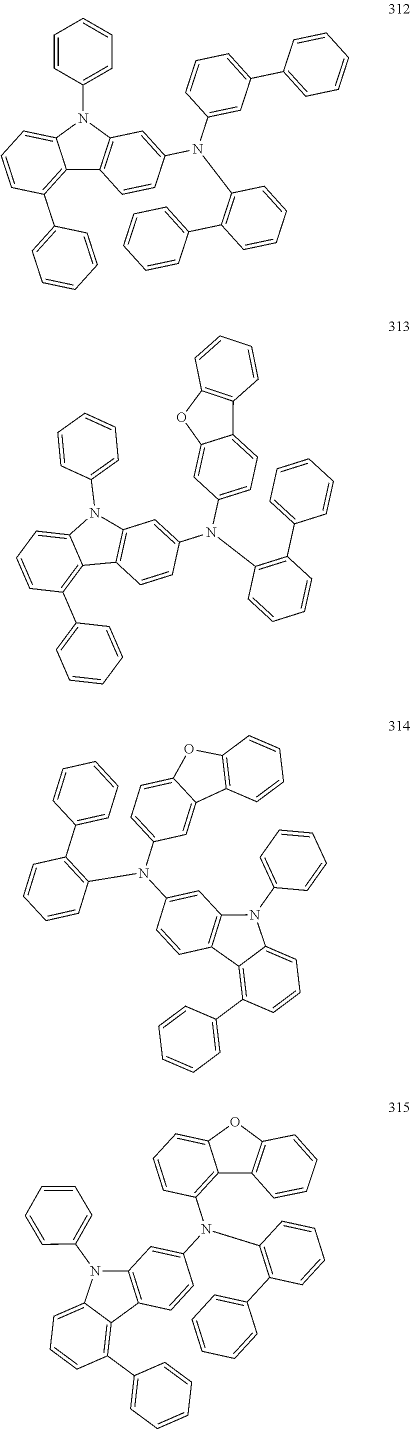 [SMILES: c1ccc(-c2cccc(N(c3ccc4c5c(-c6ccccc6)cccc5n(-c5ccccc5)c4c3)c3ccccc3-c3ccccc3)c2)cc1 ; c1ccc(-c2ccccc2N(c2ccc3c(c2)oc2ccccc23)c2ccc3c4c(-c5ccccc5)cccc4n(-c4ccccc4)c3c2)cc1 ; c1ccc(-c2ccccc2N(c2ccc3oc4ccccc4c3c2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2cccc3oc4ccccc4c23)cc1]

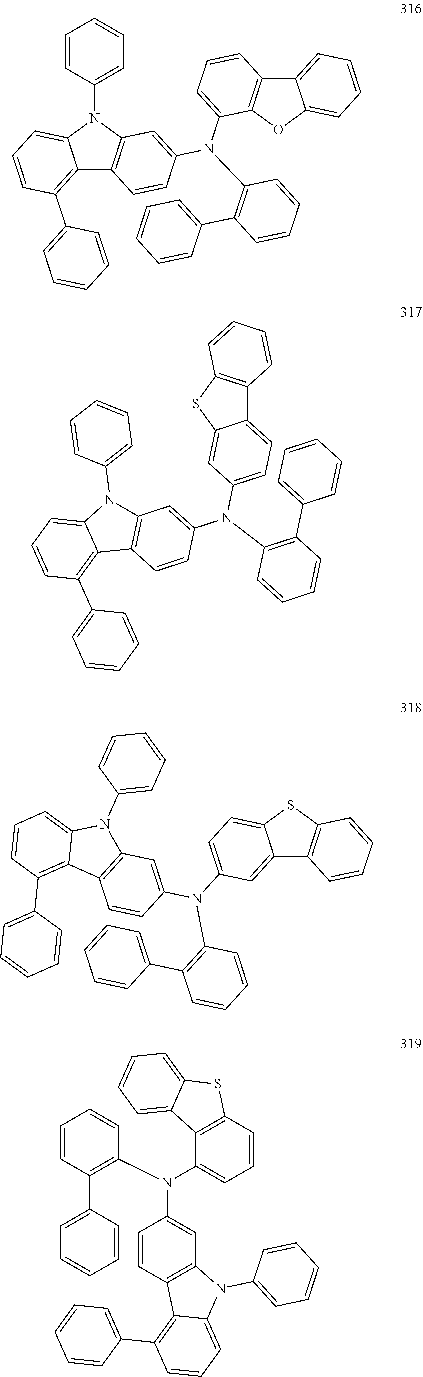 [SMILES: c1ccc(-c2ccccc2N(c2ccc3c4c(-c5ccccc5)cccc4n(-c4ccccc4)c3c2)c2cccc3c2oc2ccccc23)cc1 ; c1ccc(-c2ccccc2N(c2ccc3c(c2)sc2ccccc23)c2ccc3c4c(-c5ccccc5)cccc4n(-c4ccccc4)c3c2)cc1 ; c1ccc(-c2ccccc2N(c2ccc3sc4ccccc4c3c2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2cccc3sc4ccccc4c23)cc1]

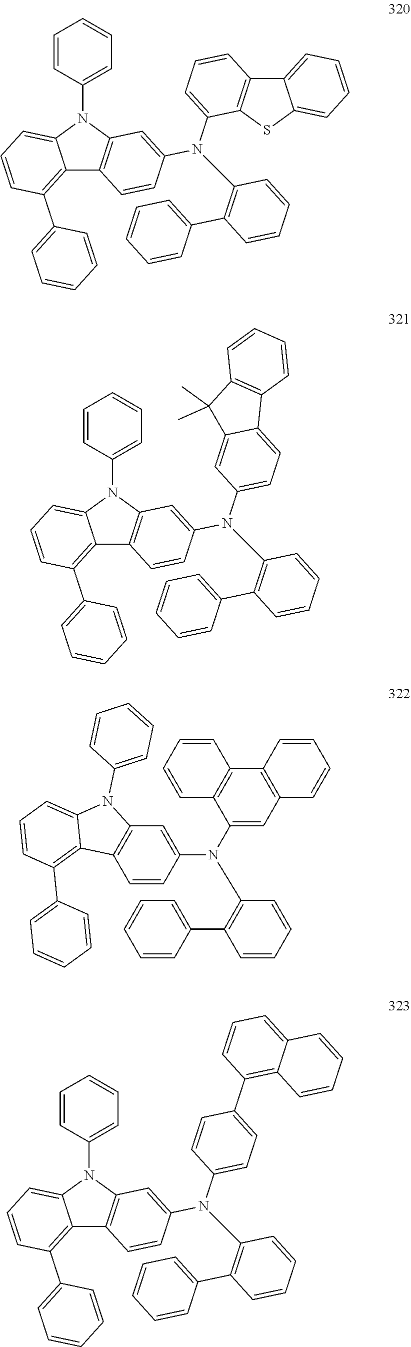

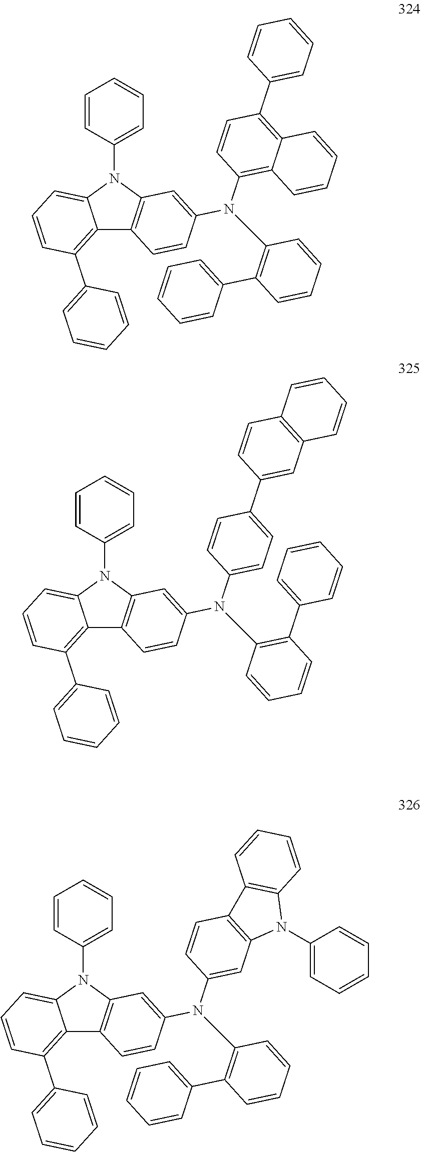 [SMILES: c1ccc(-c2ccccc2N(c2ccc3c4c(-c5ccccc5)cccc4n(-c4ccccc4)c3c2)c2ccc(-c3ccccc3)c3ccccc23)cc1 ; c1ccc(-c2ccccc2N(c2ccc(-c3ccc4ccccc4c3)cc2)c2ccc3c4c(-c5ccccc5)cccc4n(-c4ccccc4)c3c2)cc1 ; c1ccc(-c2ccccc2N(c2ccc3c4ccccc4n(-c4ccccc4)c3c2)c2ccc3c4c(-c5ccccc5)cccc4n(-c4ccccc4)c3c2)cc1]

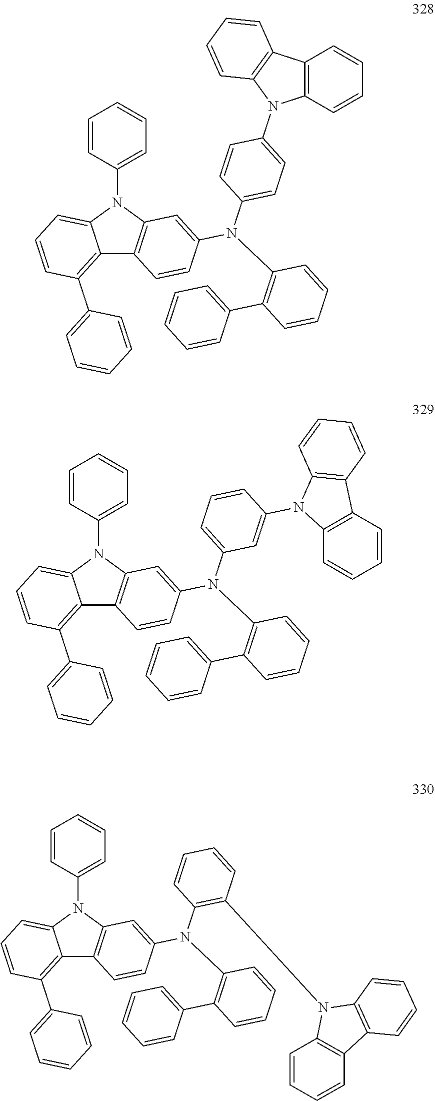 [SMILES: c1ccc(-c2ccccc2N(c2ccc(-n3c4ccccc4c4ccccc43)cc2)c2ccc3c4c(-c5ccccc5)cccc4n(-c4ccccc4)c3c2)cc1 ; c1ccc(-c2ccccc2N(c2cccc(-n3c4ccccc4c4ccccc43)c2)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2ccccc2-n2c3ccccc3c3ccccc32)cc1]

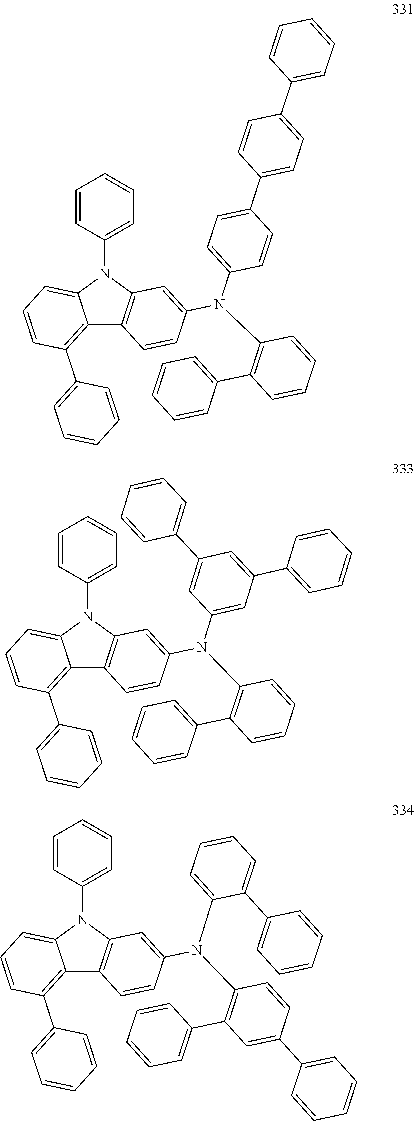

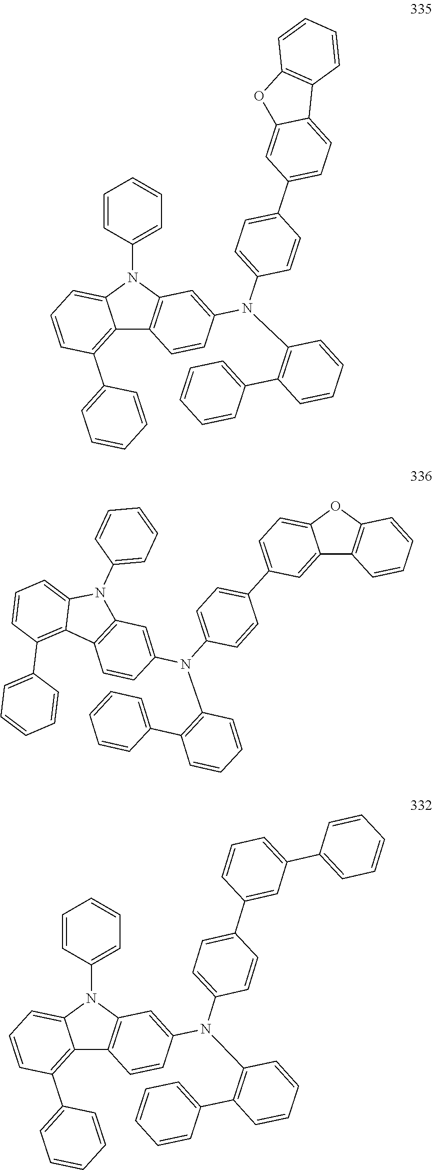 [SMILES: c1ccc(-c2ccccc2N(c2ccc(-c3ccc4c(c3)oc3ccccc34)cc2)c2ccc3c4c(-c5ccccc5)cccc4n(-c4ccccc4)c3c2)cc1 ; c1ccc(-c2ccccc2N(c2ccc(-c3ccc4oc5ccccc5c4c3)cc2)c2ccc3c4c(-c5ccccc5)cccc4n(-c4ccccc4)c3c2)cc1 ; c1ccc(-c2cccc(-c3ccc(N(c4ccc5c6c(-c7ccccc7)cccc6n(-c6ccccc6)c5c4)c4ccccc4-c4ccccc4)cc3)c2)cc1]

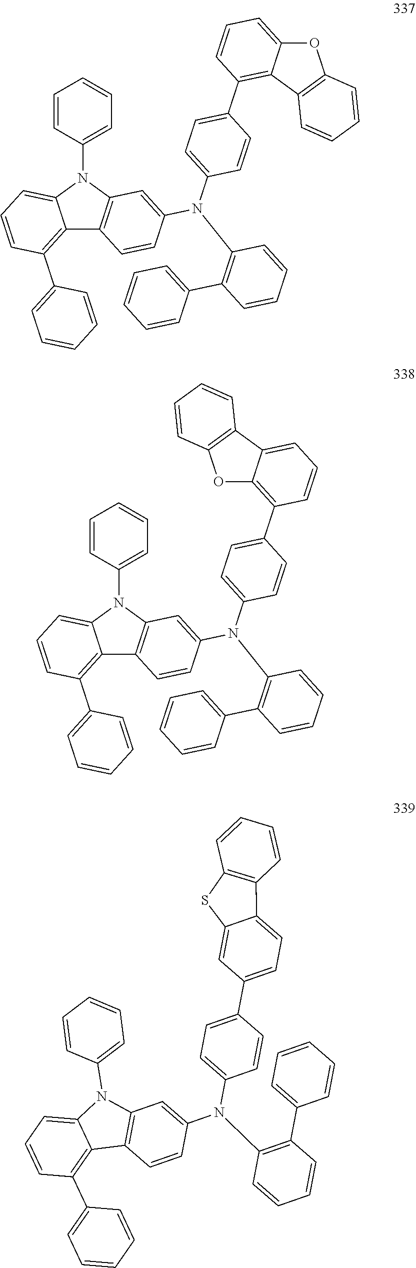 [SMILES: c1ccc(-c2ccccc2N(c2ccc(-c3cccc4oc5ccccc5c34)cc2)c2ccc3c4c(-c5ccccc5)cccc4n(-c4ccccc4)c3c2)cc1 ; c1ccc(-c2ccccc2N(c2ccc(-c3cccc4c3oc3ccccc34)cc2)c2ccc3c4c(-c5ccccc5)cccc4n(-c4ccccc4)c3c2)cc1 ; c1ccc(-c2ccccc2N(c2ccc(-c3ccc4c(c3)sc3ccccc34)cc2)c2ccc3c4c(-c5ccccc5)cccc4n(-c4ccccc4)c3c2)cc1]

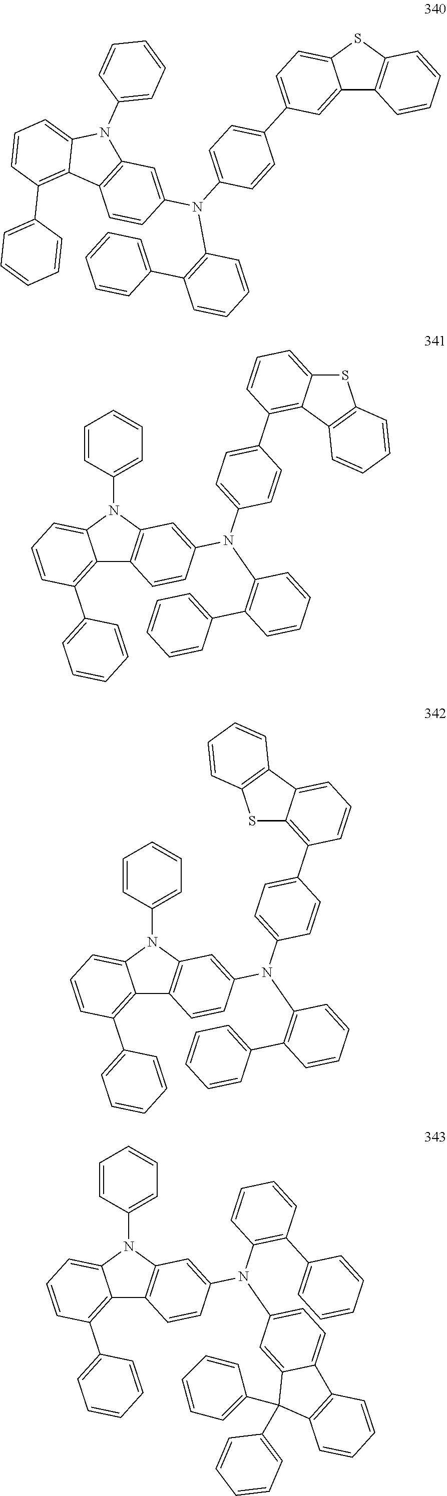

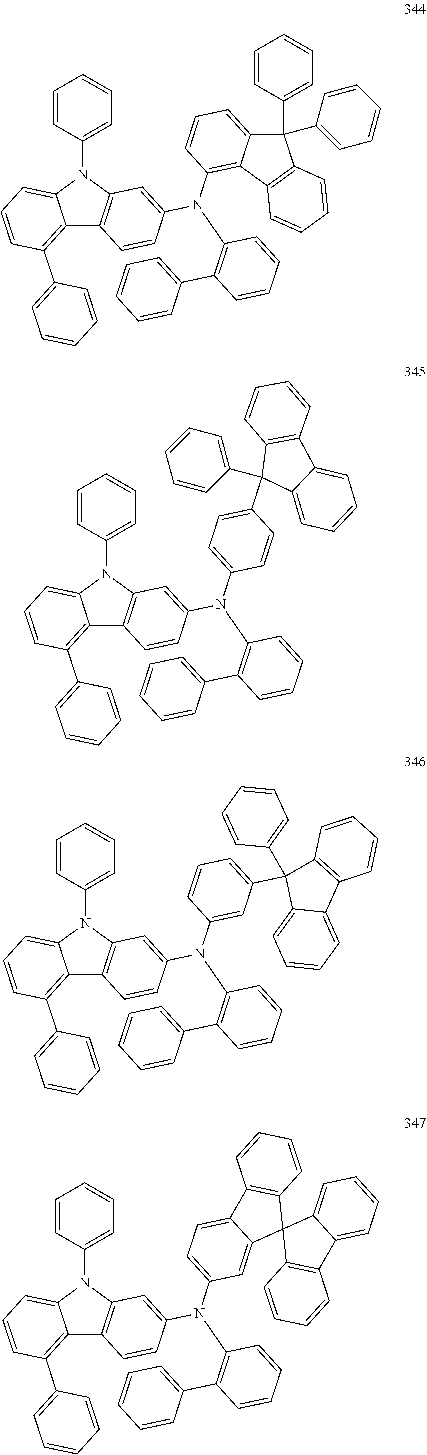 [SMILES: c1ccc(-c2ccccc2N(c2ccc3c4c(-c5ccccc5)cccc4n(-c4ccccc4)c3c2)c2cccc3c2-c2ccccc2C3(c2ccccc2)c2ccccc2)cc1 ; c1ccc(-c2ccccc2N(c2ccc(C3(c4ccccc4)c4ccccc4-c4ccccc43)cc2)c2ccc3c4c(-c5ccccc5)cccc4n(-c4ccccc4)c3c2)cc1 ; c1ccc(-c2ccccc2N(c2cccc(C3(c4ccccc4)c4ccccc4-c4ccccc43)c2)c2ccc3c4c(-c5ccccc5)cccc4n(-c4ccccc4)c3c2)cc1 ; c1ccc(-c2ccccc2N(c2ccc3c(c2)C2(c4ccccc4-c4ccccc42)c2ccccc2-3)c2ccc3c4c(-c5ccccc5)cccc4n(-c4ccccc4)c3c2)cc1]

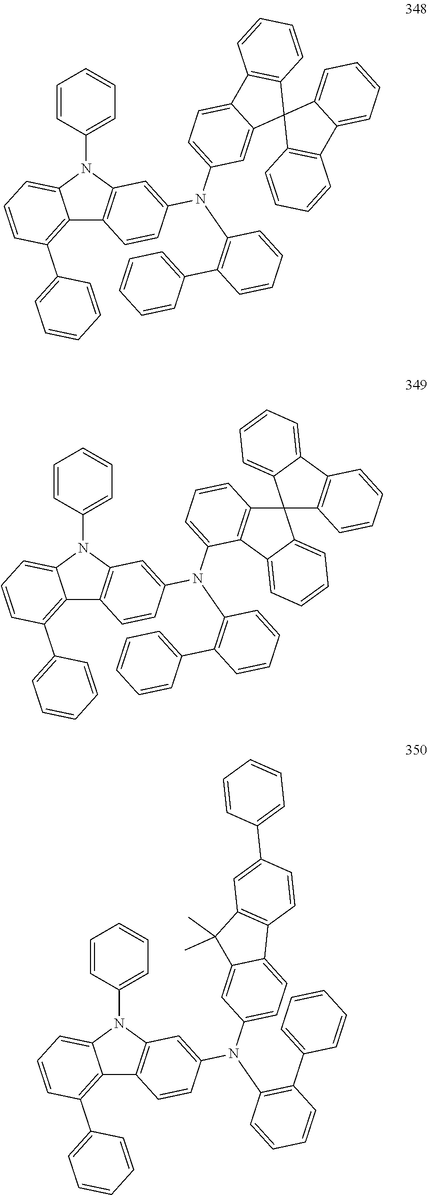 [SMILES: c1ccc(-c2ccccc2N(c2ccc3c(c2)C2(c4ccccc4-c4ccccc42)c2ccccc2-3)c2ccc3c4c(-c5ccccc5)cccc4n(-c4ccccc4)c3c2)cc1 ; c1ccc(-c2ccccc2N(c2ccc3c4c(-c5ccccc5)cccc4n(-c4ccccc4)c3c2)c2cccc3c2-c2ccccc2C32c3ccccc3-c3ccccc32)cc1 ; CC1(C)c2cc(-c3ccccc3)ccc2-c2ccc(N(c3ccc4c5c(-c6ccccc6)cccc5n(-c5ccccc5)c4c3)c3ccccc3-c3ccccc3)cc21]

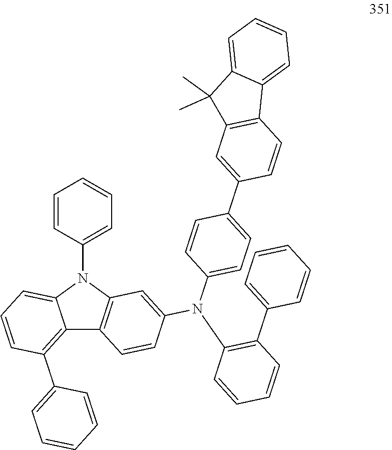 [SMILES: CC1(C)c2ccccc2-c2ccc(-c3ccc(N(c4ccc5c6c(-c7ccccc7)cccc6n(-c6ccccc6)c5c4)c4ccccc4-c4ccccc4)cc3)cc21]

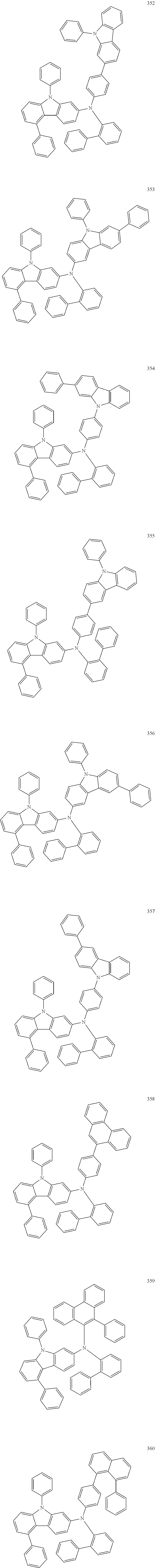

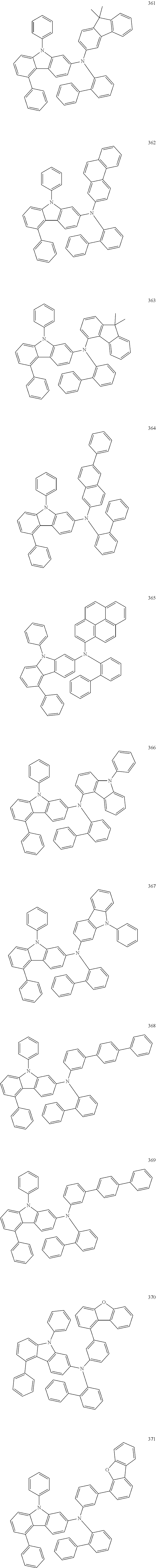

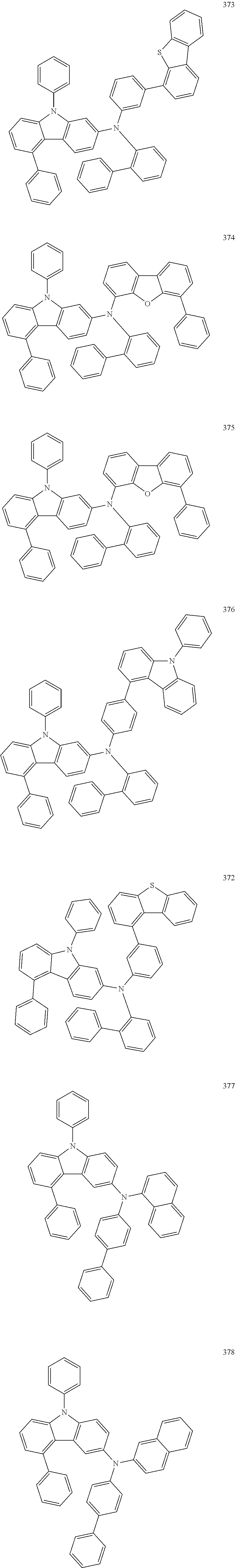

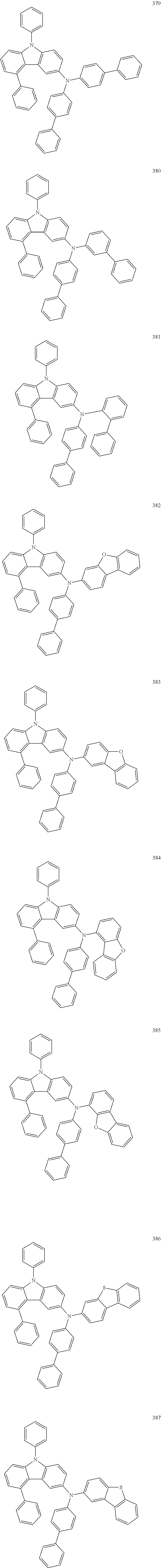

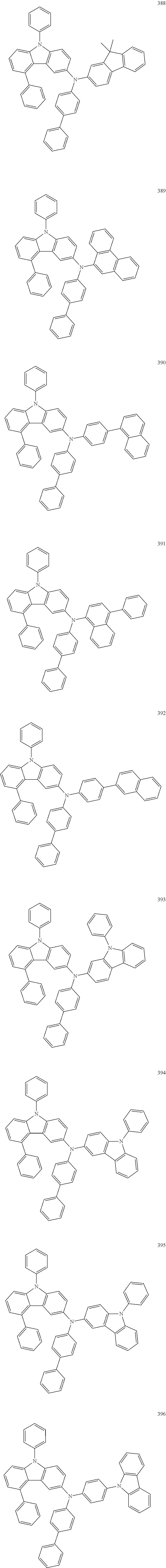

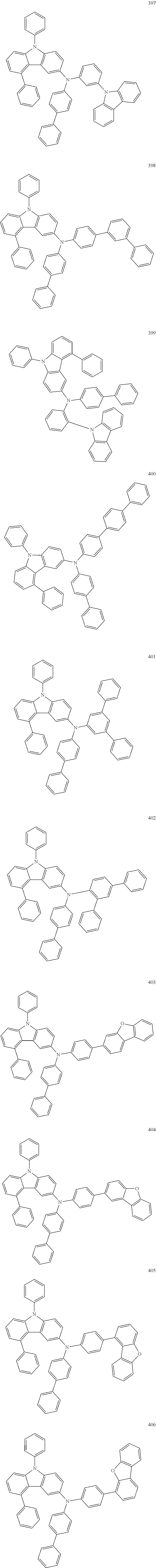

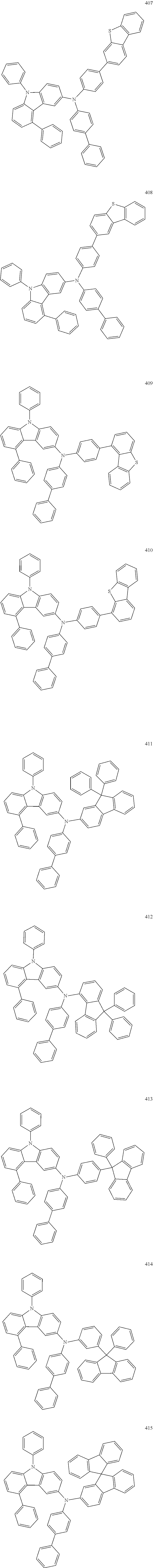

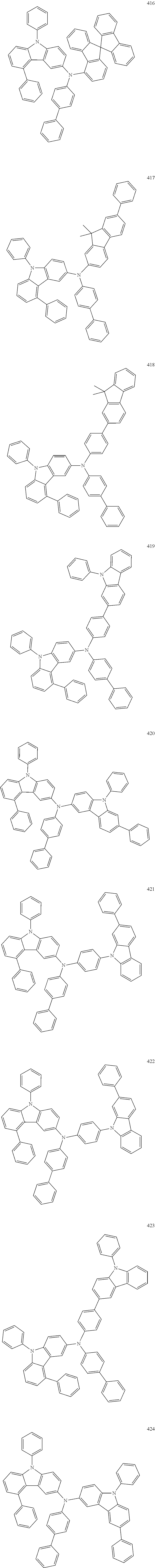

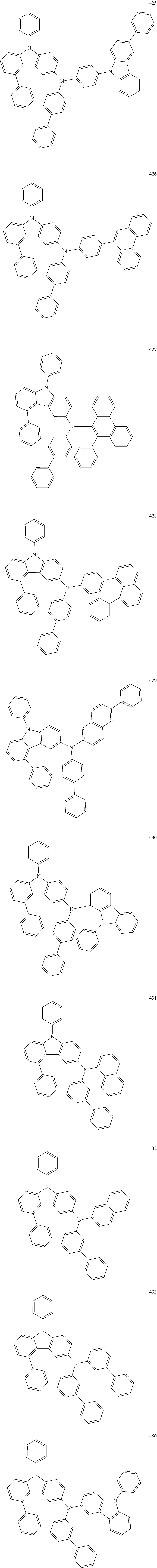

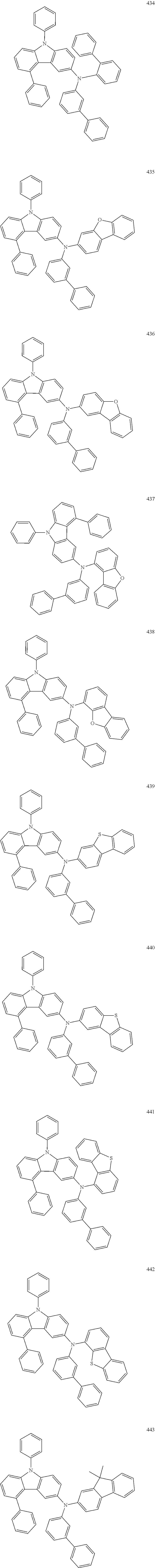

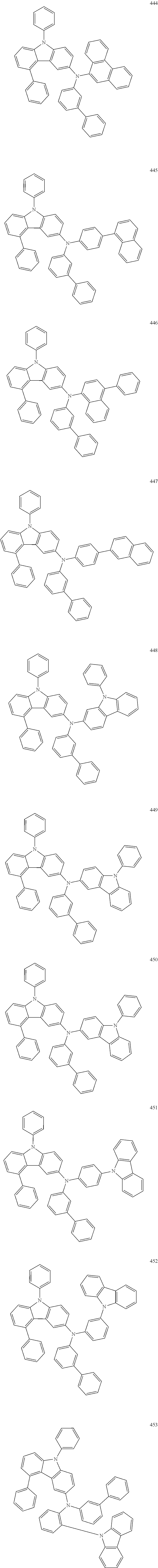

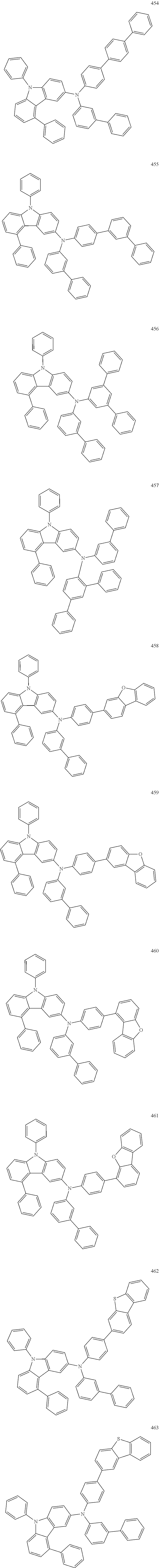

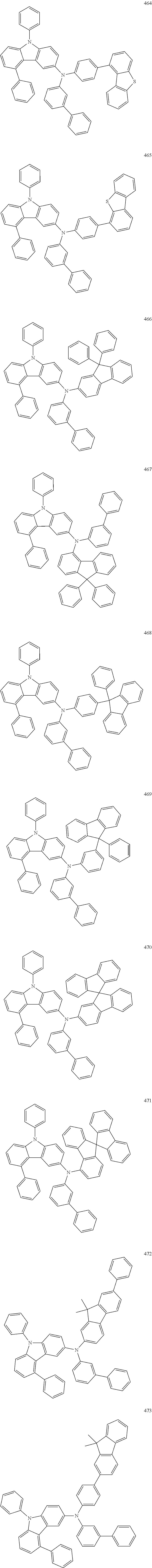

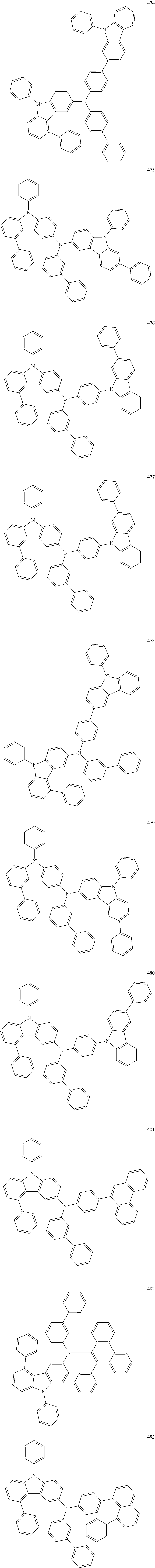

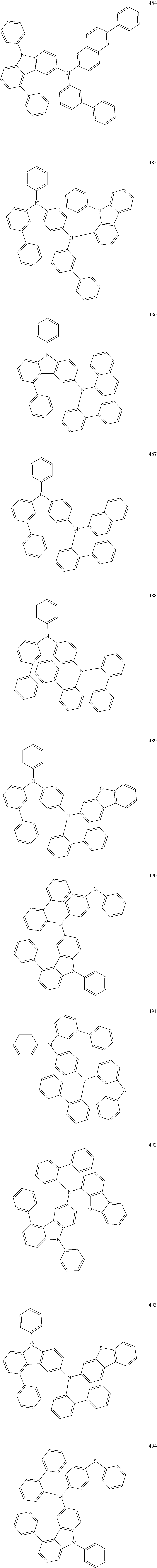

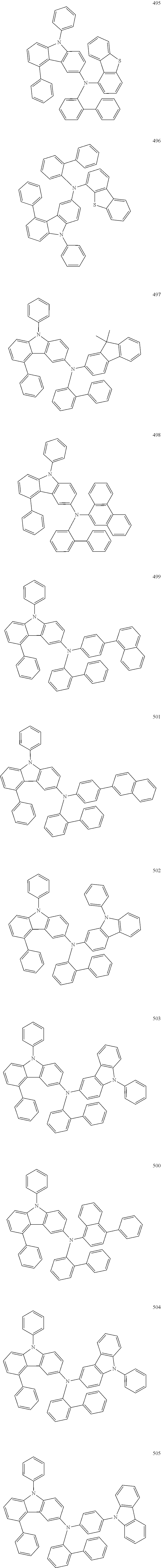

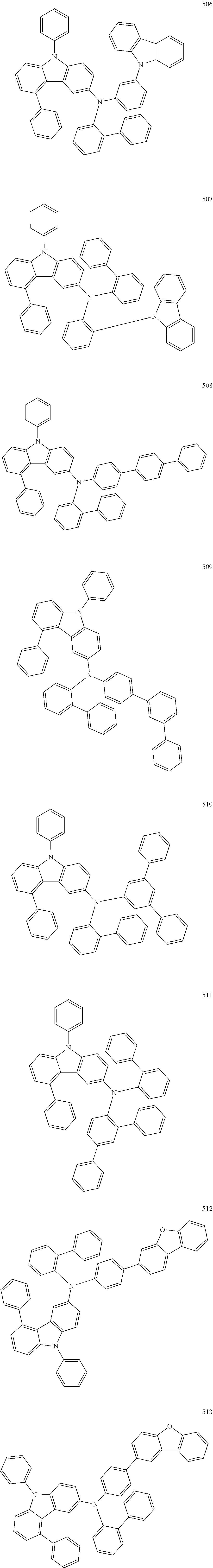

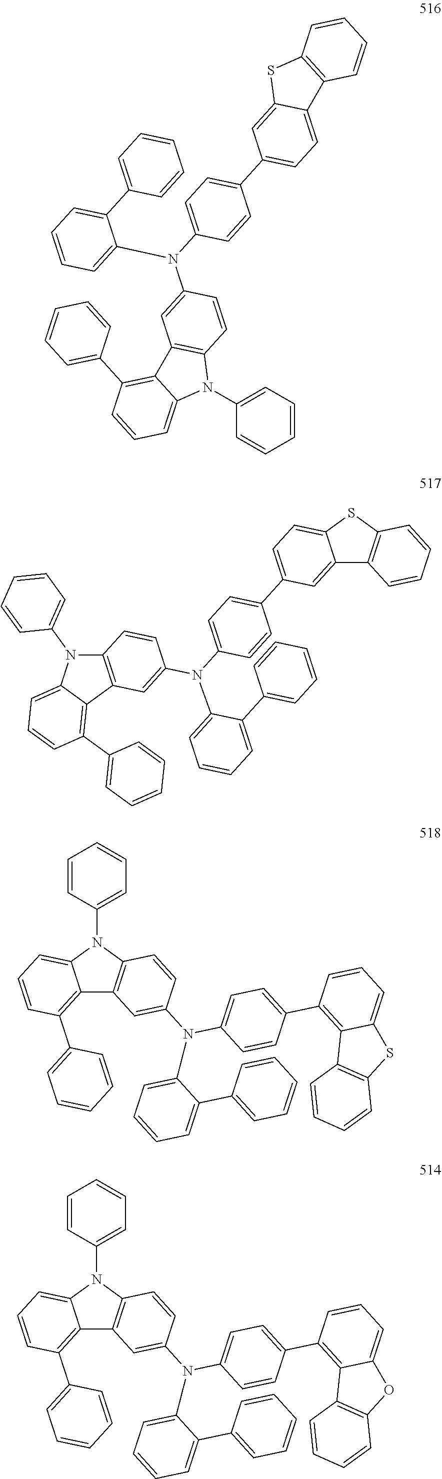 [SMILES: c1ccc(-c2ccccc2N(c2ccc(-c3ccc4c(c3)sc3ccccc34)cc2)c2ccc3c(c2)c2c(-c4ccccc4)cccc2n3-c2ccccc2)cc1 ; c1ccc(-c2ccccc2N(c2ccc(-c3ccc4sc5ccccc5c4c3)cc2)c2ccc3c(c2)c2c(-c4ccccc4)cccc2n3-c2ccccc2)cc1 ; c1ccc(-c2ccccc2N(c2ccc(-c3cccc4sc5ccccc5c34)cc2)c2ccc3c(c2)c2c(-c4ccccc4)cccc2n3-c2ccccc2)cc1 ; c1ccc(-c2ccccc2N(c2ccc(-c3cccc4oc5ccccc5c34)cc2)c2ccc3c(c2)c2c(-c4ccccc4)cccc2n3-c2ccccc2)cc1]

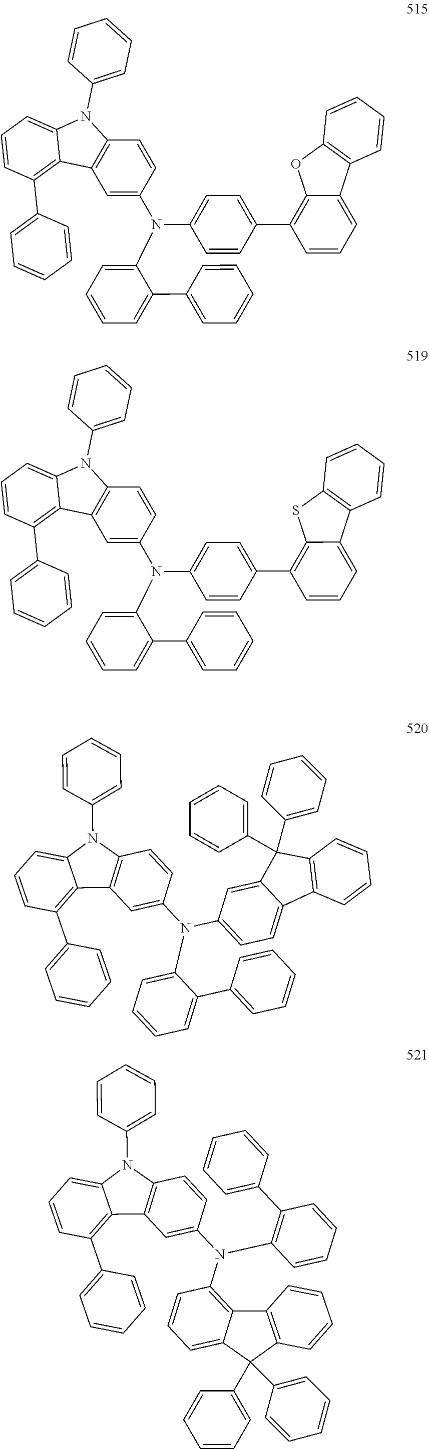

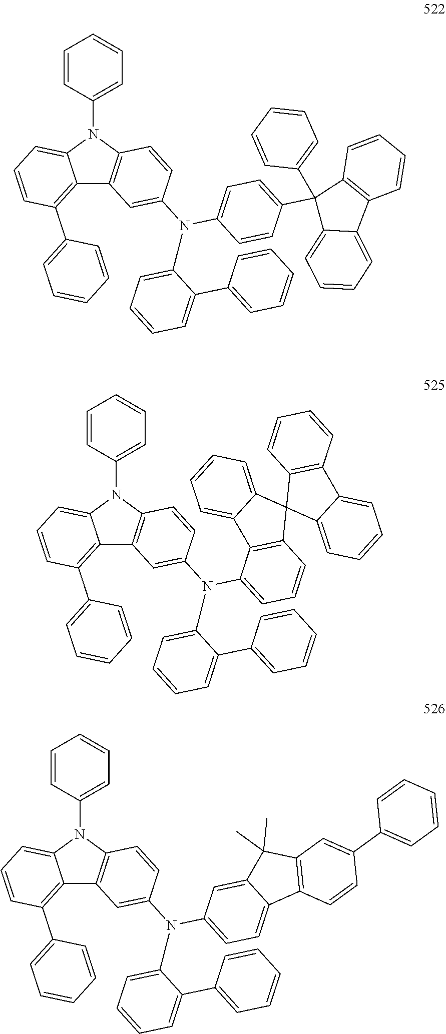 [SMILES: c1ccc(-c2ccccc2N(c2ccc(C3(c4ccccc4)c4ccccc4-c4ccccc43)cc2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2cccc3c2-c2ccccc2C32c3ccccc3-c3ccccc32)cc1 ; CC1(C)c2cc(-c3ccccc3)ccc2-c2ccc(N(c3ccc4c(c3)c3c(-c5ccccc5)cccc3n4-c3ccccc3)c3ccccc3-c3ccccc3)cc21]

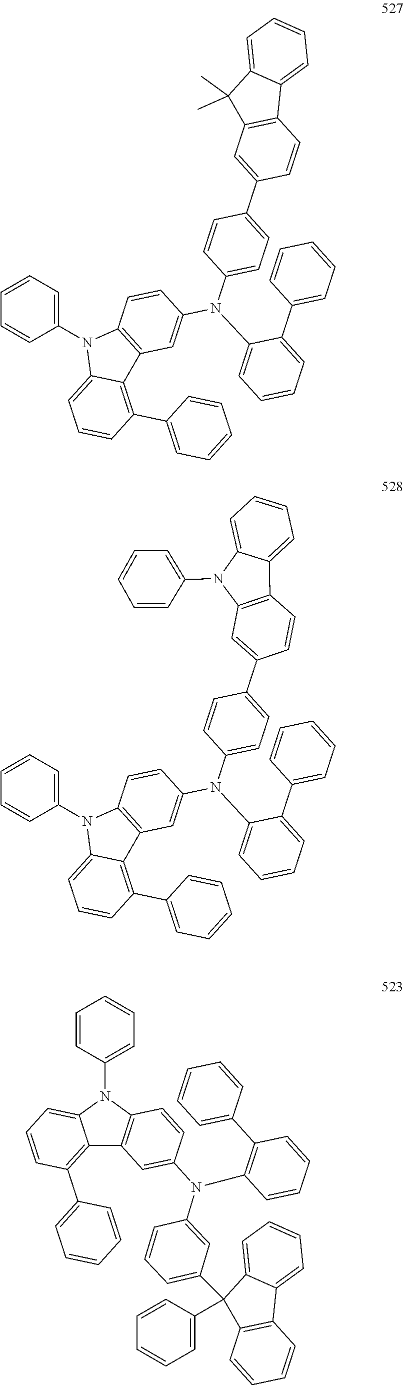 [SMILES: CC1(C)c2ccccc2-c2ccc(-c3ccc(N(c4ccc5c(c4)c4c(-c6ccccc6)cccc4n5-c4ccccc4)c4ccccc4-c4ccccc4)cc3)cc21 ; c1ccc(-c2ccccc2N(c2ccc(-c3ccc4c5ccccc5n(-c5ccccc5)c4c3)cc2)c2ccc3c(c2)c2c(-c4ccccc4)cccc2n3-c2ccccc2)cc1 ; c1ccc(-c2ccccc2N(c2cccc(C3(c4ccccc4)c4ccccc4-c4ccccc43)c2)c2ccc3c(c2)c2c(-c4ccccc4)cccc2n3-c2ccccc2)cc1]

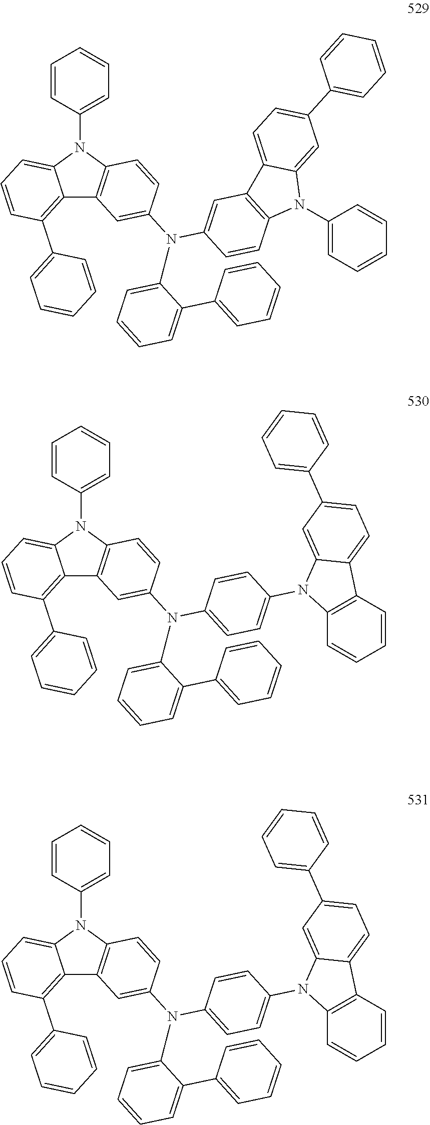 [SMILES: c1ccc(-c2ccc3c4cc(N(c5ccc6c(c5)c5c(-c7ccccc7)cccc5n6-c5ccccc5)c5ccccc5-c5ccccc5)ccc4n(-c4ccccc4)c3c2)cc1 ; c1ccc(-c2ccc3c4ccccc4n(-c4ccc(N(c5ccc6c(c5)c5c(-c7ccccc7)cccc5n6-c5ccccc5)c5ccccc5-c5ccccc5)cc4)c3c2)cc1 ; c1ccc(-c2ccc3c4ccccc4n(-c4ccc(N(c5ccc6c(c5)c5c(-c7ccccc7)cccc5n6-c5ccccc5)c5ccccc5-c5ccccc5)cc4)c3c2)cc1]

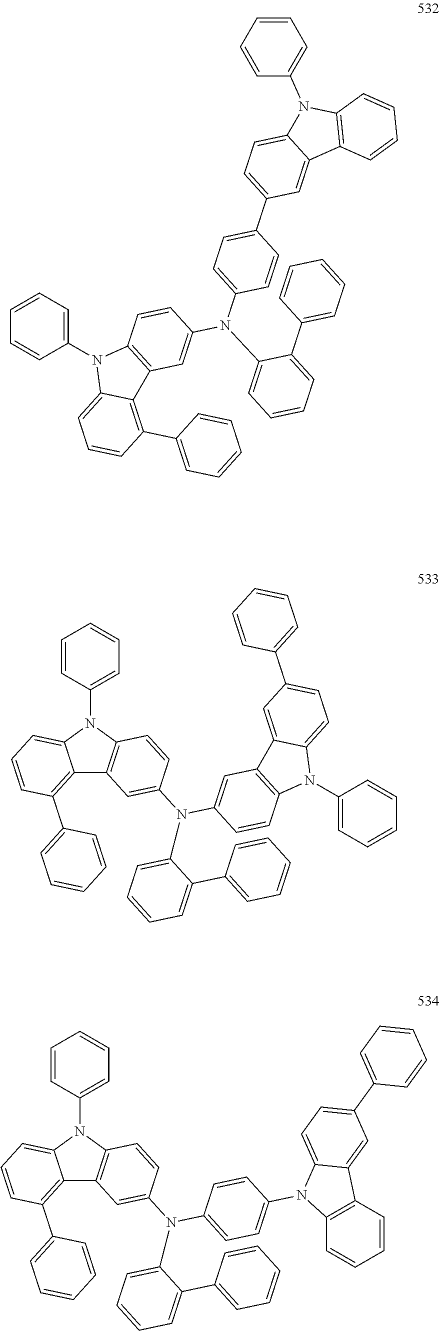 [SMILES: c1ccc(-c2ccccc2N(c2ccc(-c3ccc4c(c3)c3ccccc3n4-c3ccccc3)cc2)c2ccc3c(c2)c2c(-c4ccccc4)cccc2n3-c2ccccc2)cc1 ; c1ccc(-c2ccc3c(c2)c2cc(N(c4ccc5c(c4)c4c(-c6ccccc6)cccc4n5-c4ccccc4)c4ccccc4-c4ccccc4)ccc2n3-c2ccccc2)cc1 ; c1ccc(-c2ccc3c(c2)c2ccccc2n3-c2ccc(N(c3ccc4c(c3)c3c(-c5ccccc5)cccc3n4-c3ccccc3)c3ccccc3-c3ccccc3)cc2)cc1]

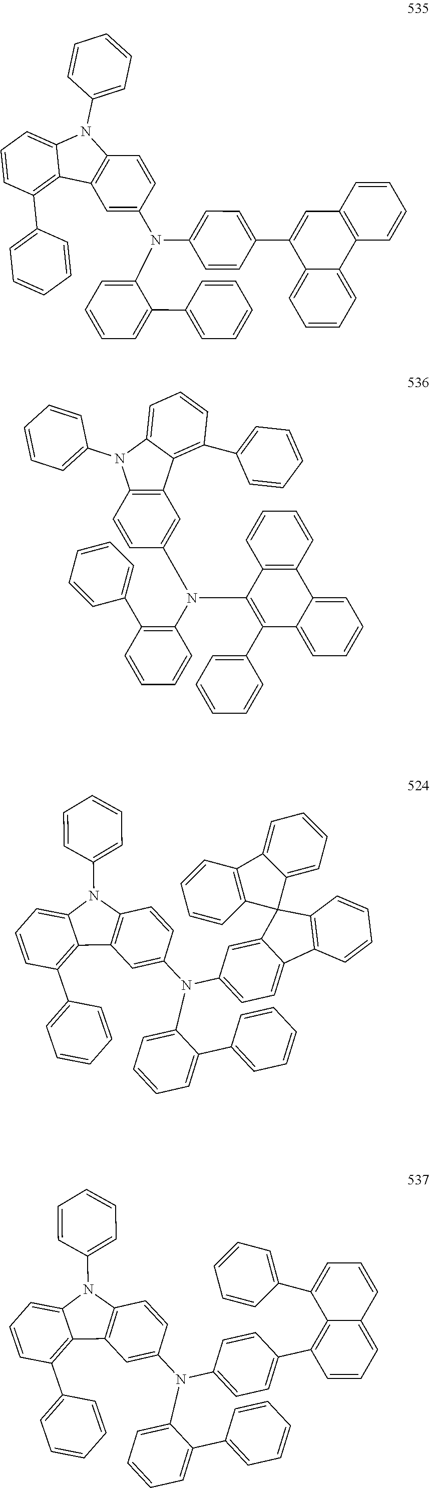 [SMILES: c1ccc(-c2ccccc2N(c2ccc(-c3cc4ccccc4c4ccccc34)cc2)c2ccc3c(c2)c2c(-c4ccccc4)cccc2n3-c2ccccc2)cc1 ; c1ccc(-c2ccccc2N(c2ccc3c(c2)c2c(-c4ccccc4)cccc2n3-c2ccccc2)c2c(-c3ccccc3)c3ccccc3c3ccccc23)cc1 ; c1ccc(-c2ccccc2N(c2ccc3c(c2)C2(c4ccccc4-c4ccccc42)c2ccccc2-3)c2ccc3c(c2)c2c(-c4ccccc4)cccc2n3-c2ccccc2)cc1 ; c1ccc(-c2ccccc2N(c2ccc(-c3cccc4cccc(-c5ccccc5)c34)cc2)c2ccc3c(c2)c2c(-c4ccccc4)cccc2n3-c2ccccc2)cc1]

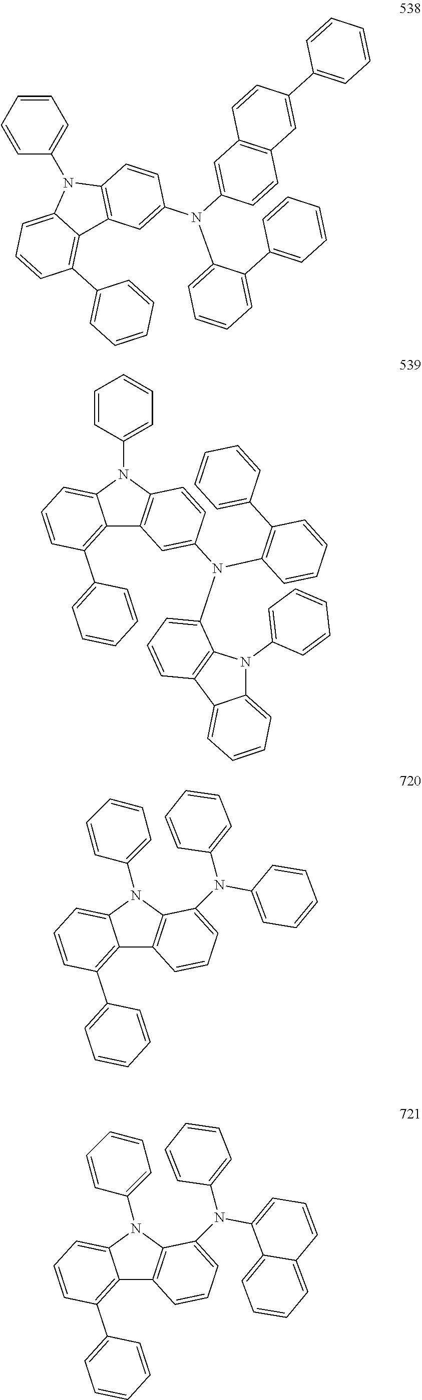

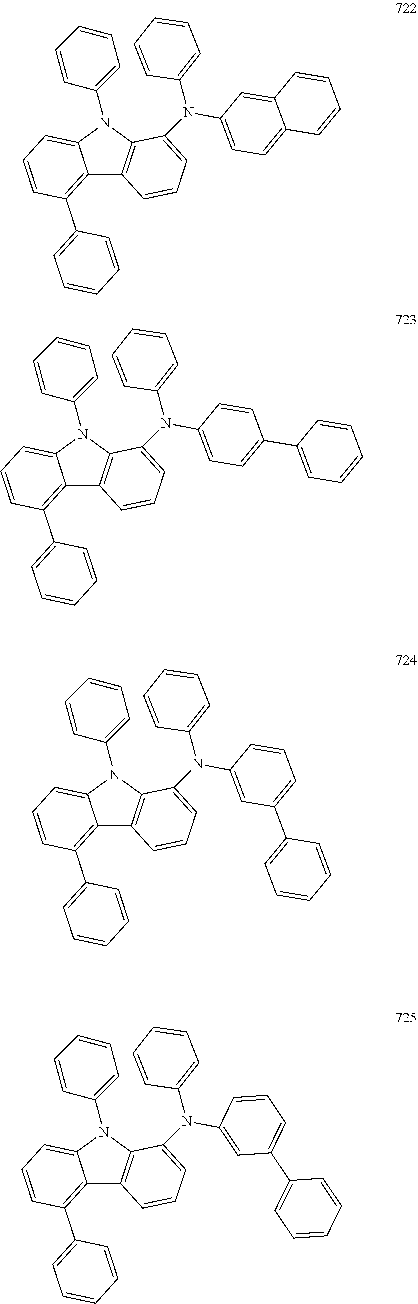 [SMILES: c1ccc(-c2cccc3c2c2cccc(N(c4ccccc4)c4ccc5ccccc5c4)c2n3-c2ccccc2)cc1 ; c1ccc(-c2ccc(N(c3ccccc3)c3cccc4c5c(-c6ccccc6)cccc5n(-c5ccccc5)c34)cc2)cc1 ; c1ccc(-c2cccc(N(c3ccccc3)c3cccc4c5c(-c6ccccc6)cccc5n(-c5ccccc5)c34)c2)cc1 ; c1ccc(-c2cccc(N(c3ccccc3)c3cccc4c5c(-c6ccccc6)cccc5n(-c5ccccc5)c34)c2)cc1]

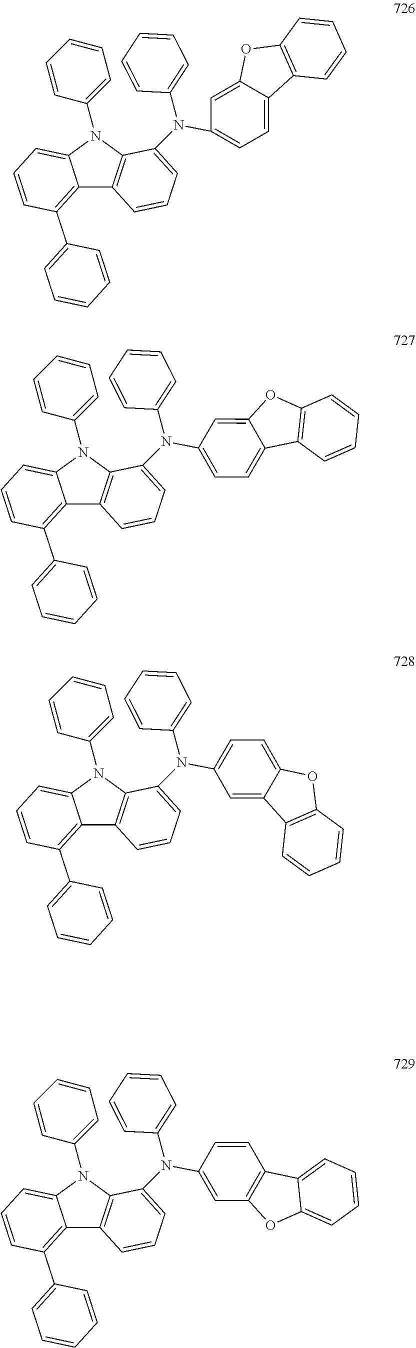 [SMILES: c1ccc(-c2cccc3c2c2cccc(N(c4ccccc4)c4ccc5c(c4)oc4ccccc45)c2n3-c2ccccc2)cc1 ; c1ccc(-c2cccc3c2c2cccc(N(c4ccccc4)c4ccc5c(c4)oc4ccccc45)c2n3-c2ccccc2)cc1 ; c1ccc(-c2cccc3c2c2cccc(N(c4ccccc4)c4ccc5oc6ccccc6c5c4)c2n3-c2ccccc2)cc1 ; c1ccc(-c2cccc3c2c2cccc(N(c4ccccc4)c4ccc5c(c4)oc4ccccc45)c2n3-c2ccccc2)cc1]

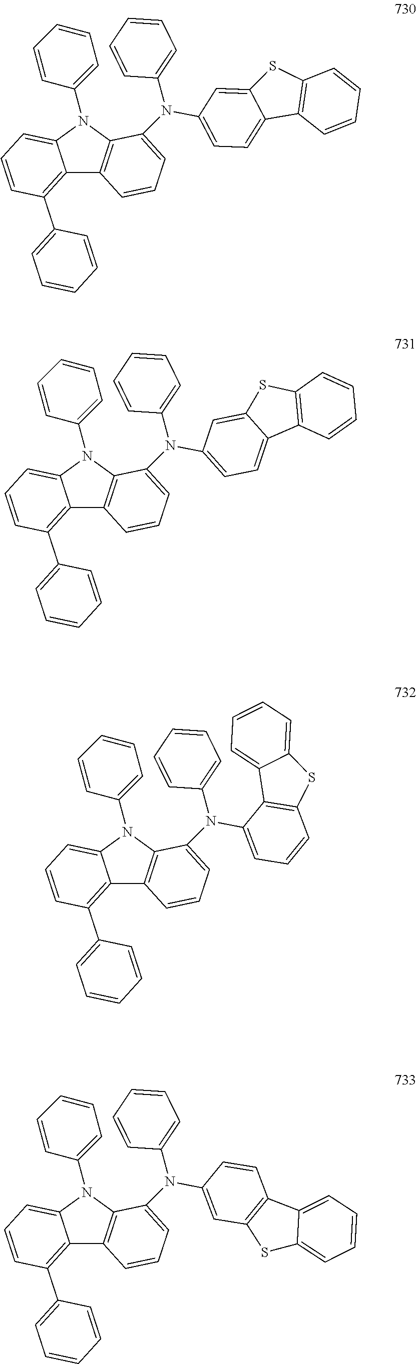 [SMILES: c1ccc(-c2cccc3c2c2cccc(N(c4ccccc4)c4ccc5c(c4)sc4ccccc45)c2n3-c2ccccc2)cc1 ; c1ccc(-c2cccc3c2c2cccc(N(c4ccccc4)c4ccc5c(c4)sc4ccccc45)c2n3-c2ccccc2)cc1 ; c1ccc(-c2cccc3c2c2cccc(N(c4ccccc4)c4cccc5sc6ccccc6c45)c2n3-c2ccccc2)cc1 ; c1ccc(-c2cccc3c2c2cccc(N(c4ccccc4)c4ccc5c(c4)sc4ccccc45)c2n3-c2ccccc2)cc1]

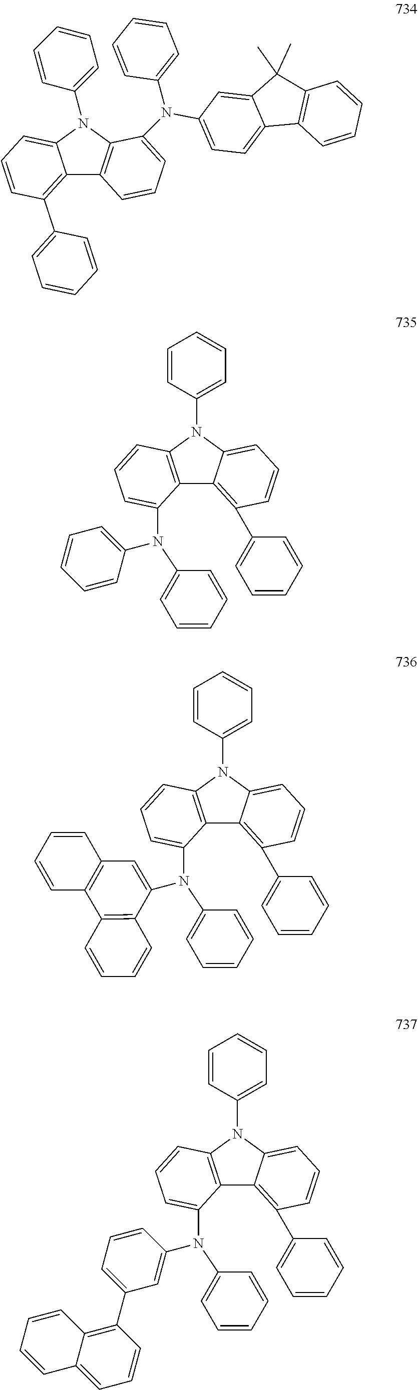

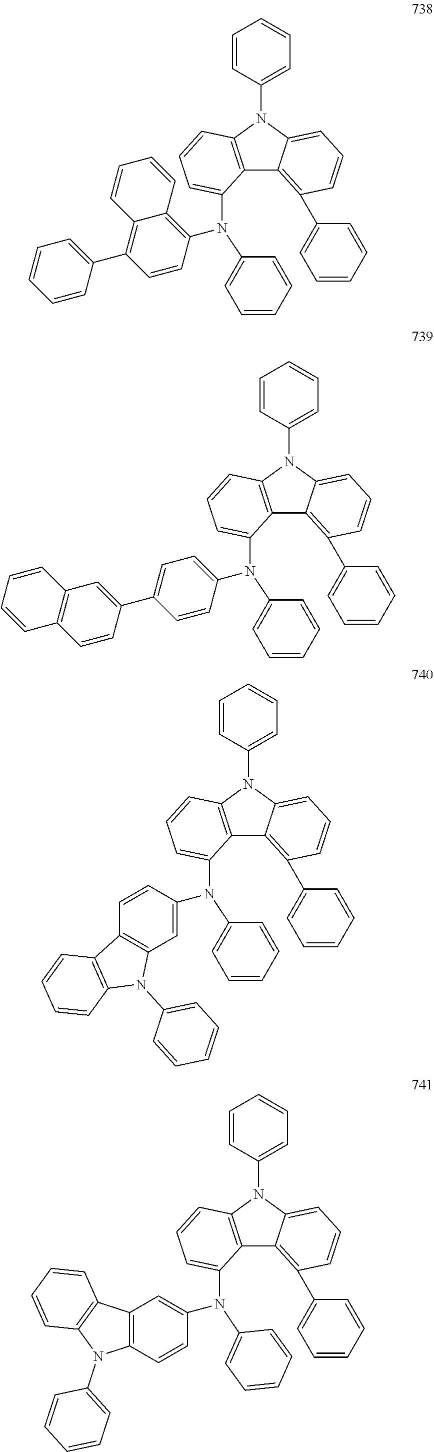

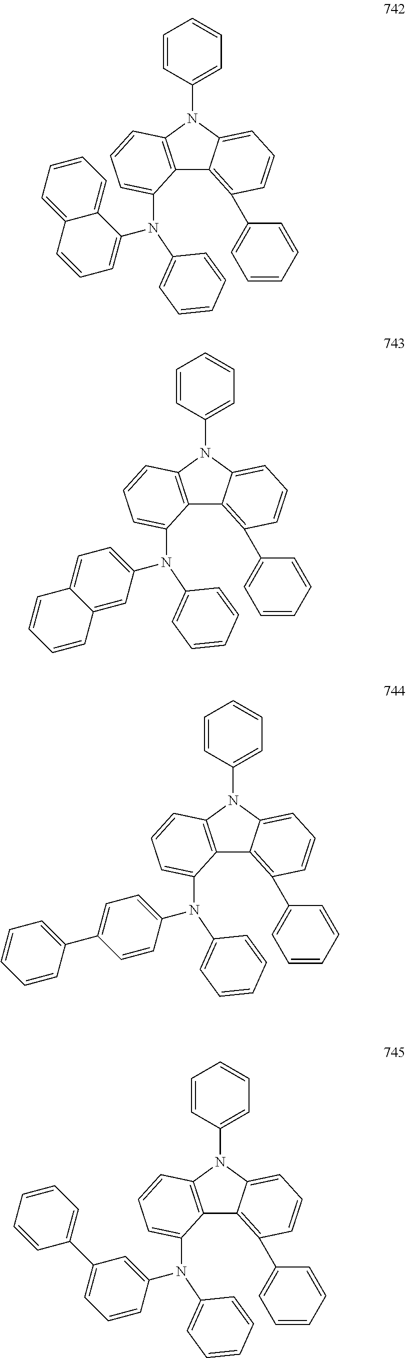

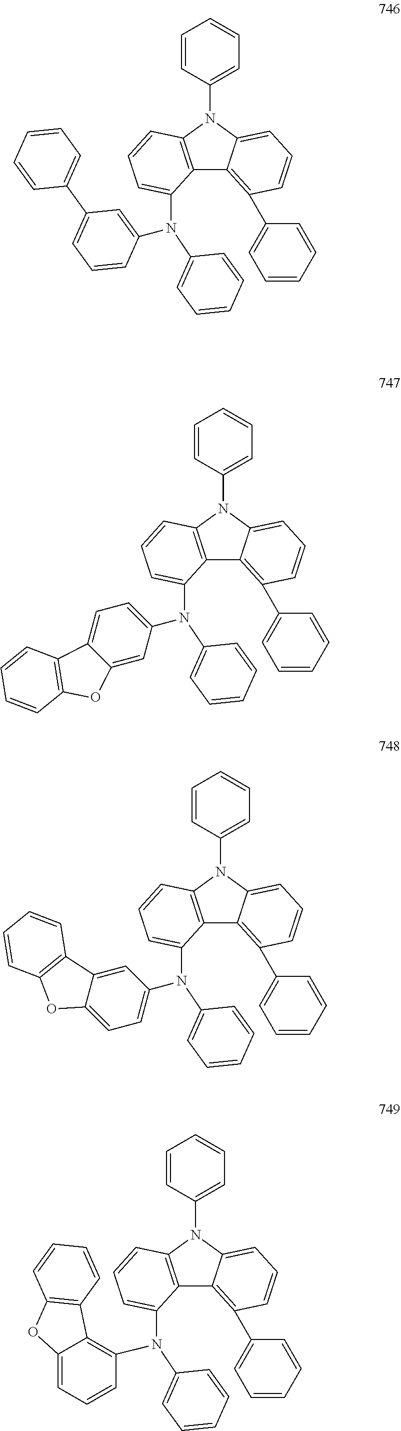 [SMILES: c1ccc(-c2cccc(N(c3ccccc3)c3cccc4c3c3c(-c5ccccc5)cccc3n4-c3ccccc3)c2)cc1 ; c1ccc(-c2cccc3c2c2c(N(c4ccccc4)c4ccc5c(c4)oc4ccccc45)cccc2n3-c2ccccc2)cc1 ; c1ccc(-c2cccc3c2c2c(N(c4ccccc4)c4ccc5oc6ccccc6c5c4)cccc2n3-c2ccccc2)cc1 ; c1ccc(-c2cccc3c2c2c(N(c4ccccc4)c4cccc5oc6ccccc6c45)cccc2n3-c2ccccc2)cc1]

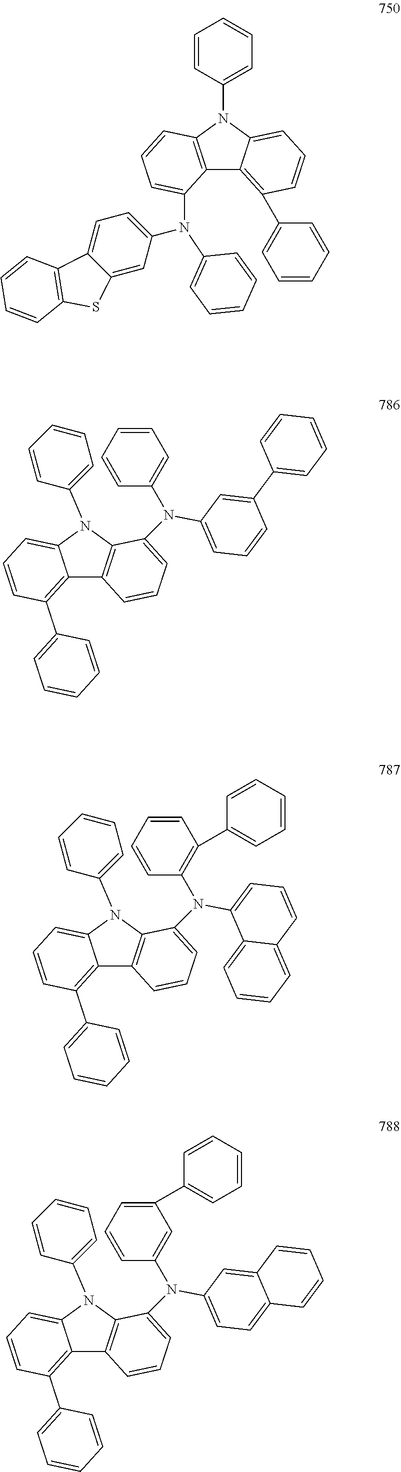 [SMILES: c1ccc(-c2cccc3c2c2c(N(c4ccccc4)c4ccc5c(c4)sc4ccccc45)cccc2n3-c2ccccc2)cc1 ; c1ccc(-c2cccc(N(c3ccccc3)c3cccc4c5c(-c6ccccc6)cccc5n(-c5ccccc5)c34)c2)cc1 ; c1ccc(-c2ccccc2N(c2cccc3ccccc23)c2cccc3c4c(-c5ccccc5)cccc4n(-c4ccccc4)c23)cc1 ; c1ccc(-c2cccc(N(c3ccc4ccccc4c3)c3cccc4c5c(-c6ccccc6)cccc5n(-c5ccccc5)c34)c2)cc1]

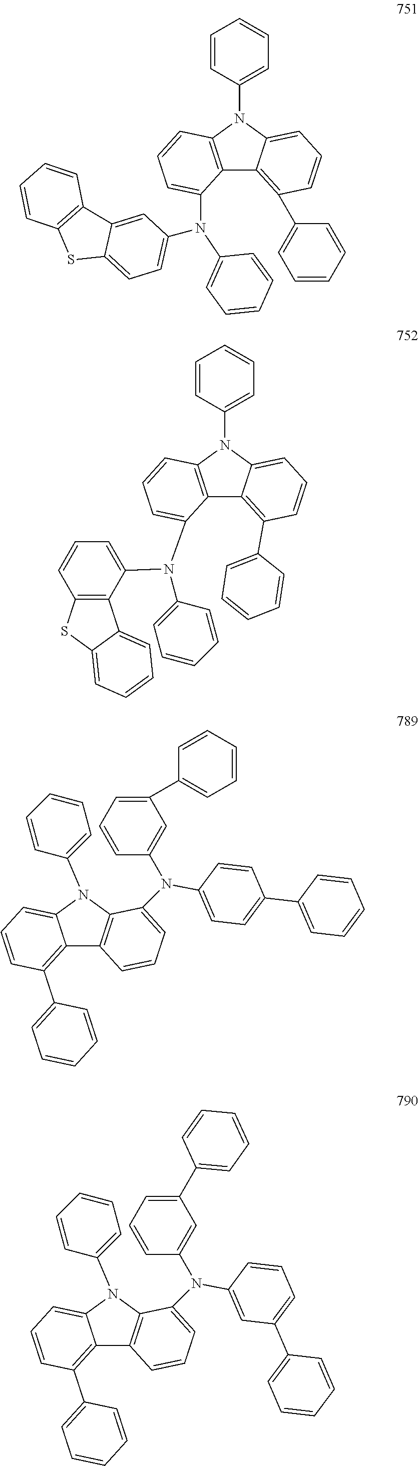

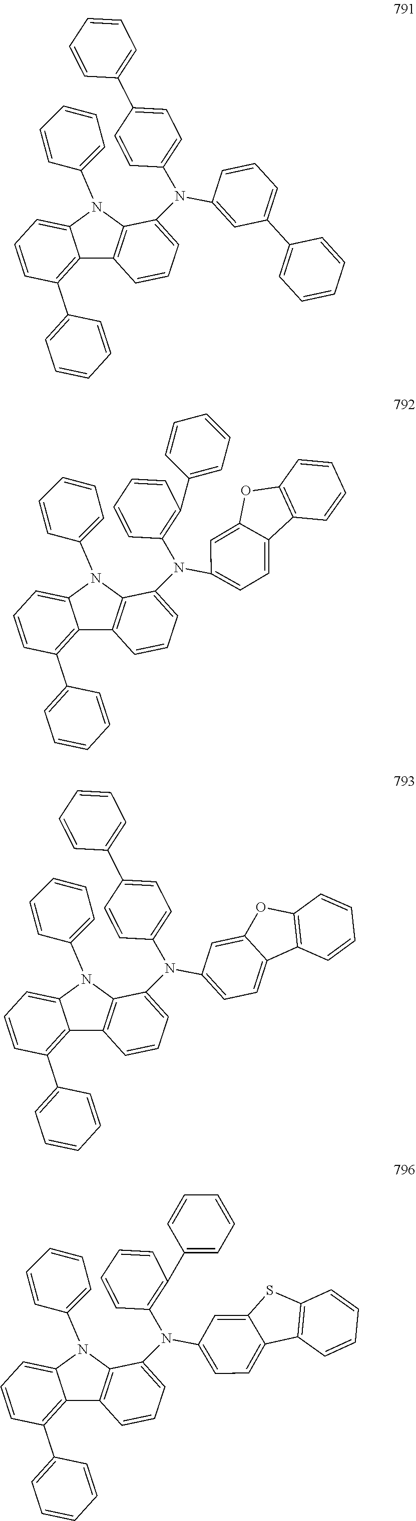

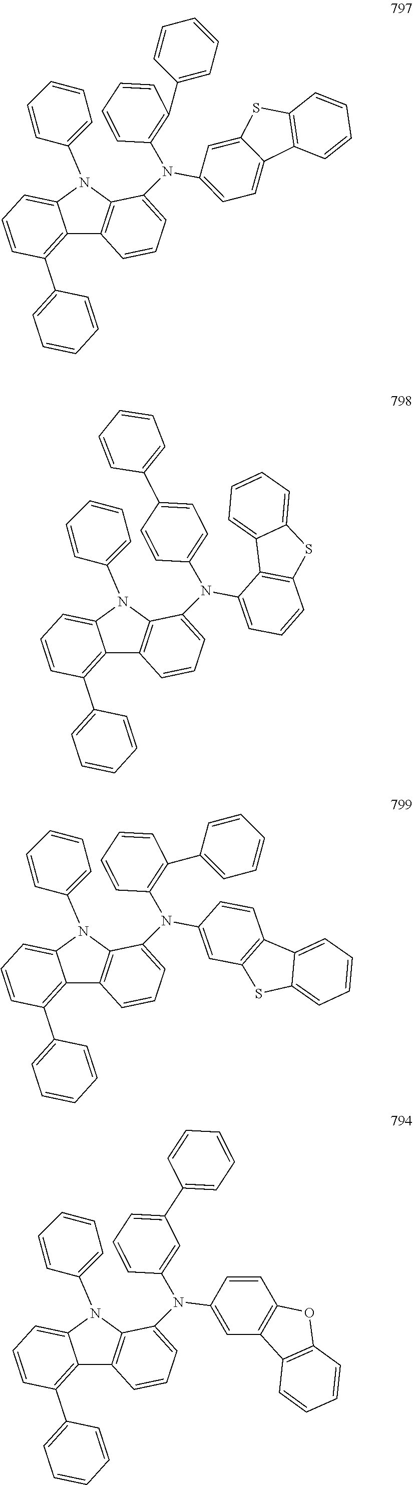 [SMILES: c1ccc(-c2ccccc2N(c2ccc3c(c2)sc2ccccc23)c2cccc3c4c(-c5ccccc5)cccc4n(-c4ccccc4)c23)cc1 ; c1ccc(-c2ccc(N(c3cccc4sc5ccccc5c34)c3cccc4c5c(-c6ccccc6)cccc5n(-c5ccccc5)c34)cc2)cc1 ; c1ccc(-c2ccccc2N(c2ccc3c(c2)sc2ccccc23)c2cccc3c4c(-c5ccccc5)cccc4n(-c4ccccc4)c23)cc1 ; c1ccc(-c2cccc(N(c3ccc4oc5ccccc5c4c3)c3cccc4c5c(-c6ccccc6)cccc5n(-c5ccccc5)c34)c2)cc1]

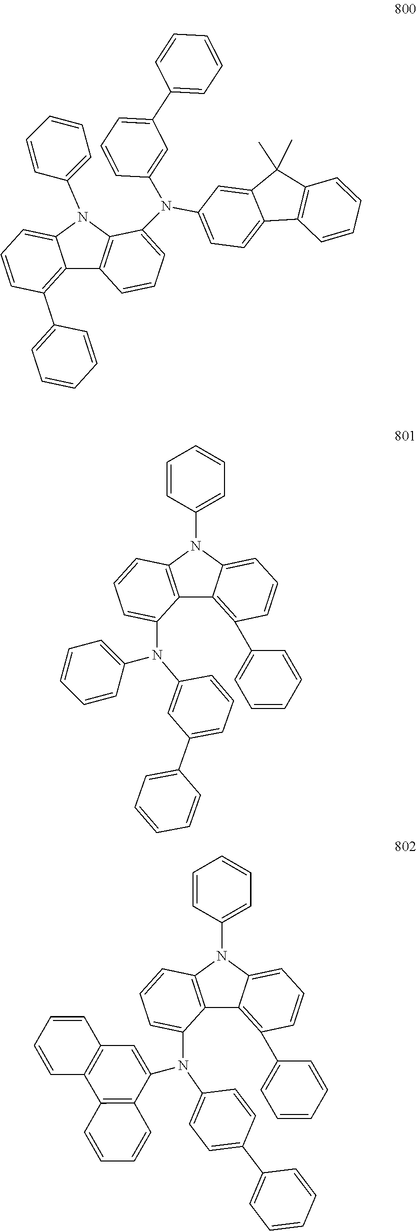

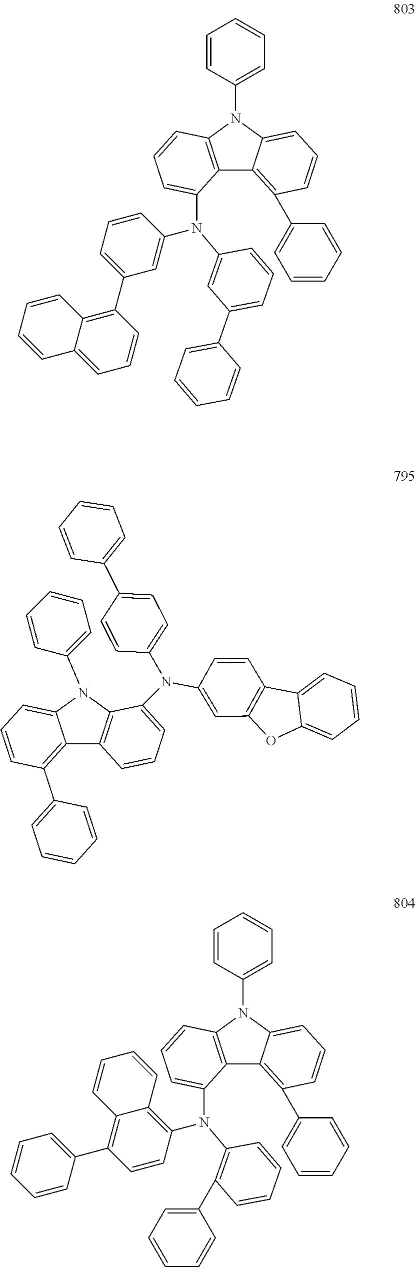 [SMILES: c1ccc(-c2cccc(N(c3cccc(-c4cccc5ccccc45)c3)c3cccc4c3c3c(-c5ccccc5)cccc3n4-c3ccccc3)c2)cc1 ; c1ccc(-c2ccc(N(c3ccc4c(c3)oc3ccccc34)c3cccc4c5c(-c6ccccc6)cccc5n(-c5ccccc5)c34)cc2)cc1 ; c1ccc(-c2ccccc2N(c2ccc(-c3ccccc3)c3ccccc23)c2cccc3c2c2c(-c4ccccc4)cccc2n3-c2ccccc2)cc1]

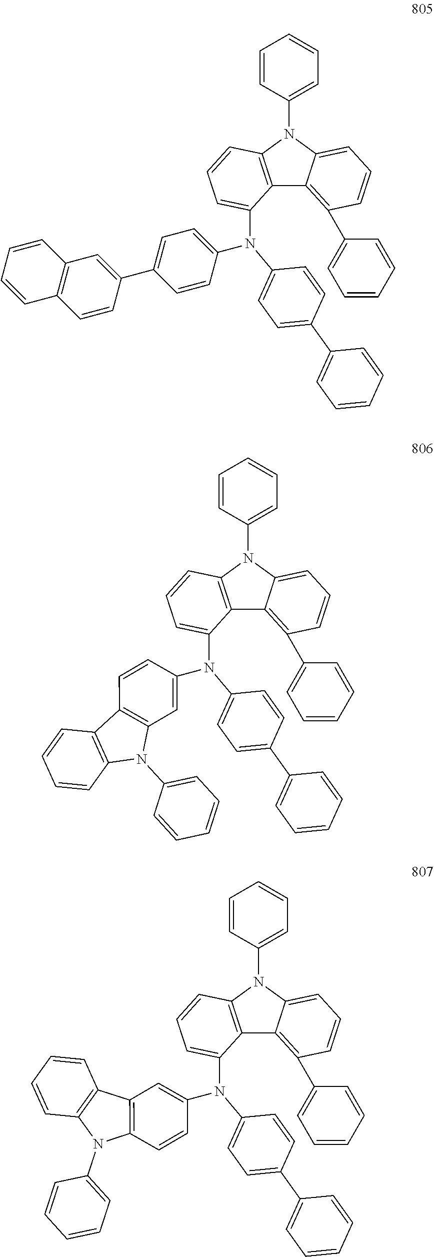 [SMILES: c1ccc(-c2ccc(N(c3ccc(-c4ccc5ccccc5c4)cc3)c3cccc4c3c3c(-c5ccccc5)cccc3n4-c3ccccc3)cc2)cc1 ; c1ccc(-c2ccc(N(c3ccc4c5ccccc5n(-c5ccccc5)c4c3)c3cccc4c3c3c(-c5ccccc5)cccc3n4-c3ccccc3)cc2)cc1 ; c1ccc(-c2ccc(N(c3ccc4c(c3)c3ccccc3n4-c3ccccc3)c3cccc4c3c3c(-c5ccccc5)cccc3n4-c3ccccc3)cc2)cc1]

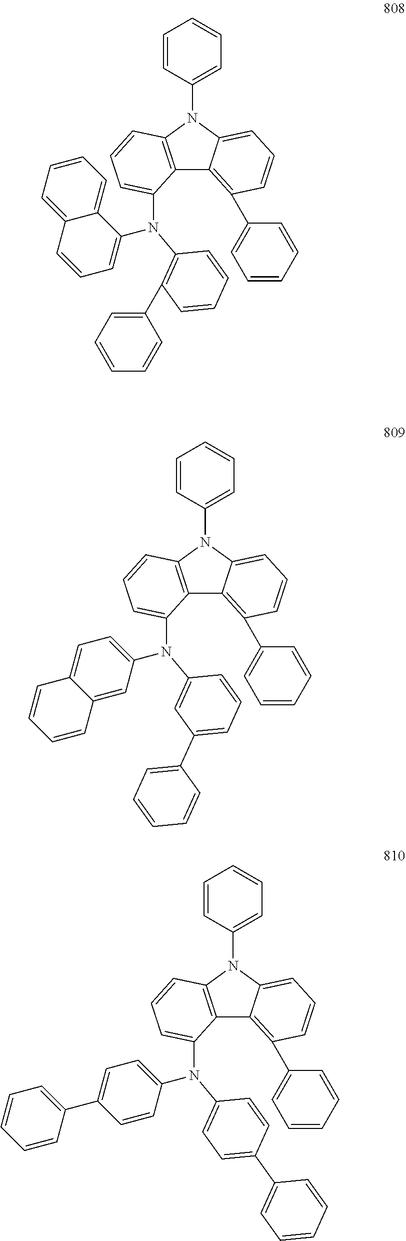 [SMILES: c1ccc(-c2ccccc2N(c2cccc3ccccc23)c2cccc3c2c2c(-c4ccccc4)cccc2n3-c2ccccc2)cc1 ; c1ccc(-c2cccc(N(c3ccc4ccccc4c3)c3cccc4c3c3c(-c5ccccc5)cccc3n4-c3ccccc3)c2)cc1 ; c1ccc(-c2ccc(N(c3ccc(-c4ccccc4)cc3)c3cccc4c3c3c(-c5ccccc5)cccc3n4-c3ccccc3)cc2)cc1]

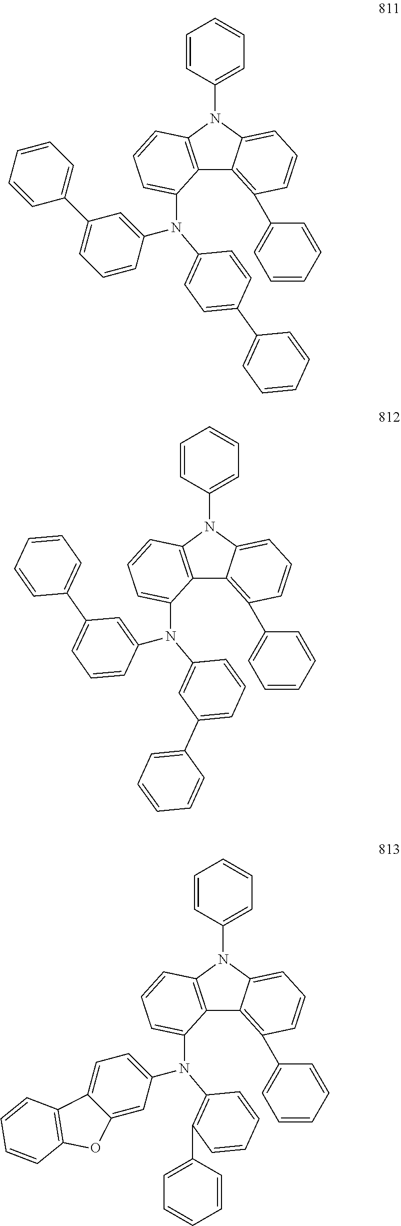 [SMILES: c1ccc(-c2ccc(N(c3cccc(-c4ccccc4)c3)c3cccc4c3c3c(-c5ccccc5)cccc3n4-c3ccccc3)cc2)cc1 ; c1ccc(-c2cccc(N(c3cccc(-c4ccccc4)c3)c3cccc4c3c3c(-c5ccccc5)cccc3n4-c3ccccc3)c2)cc1 ; c1ccc(-c2ccccc2N(c2ccc3c(c2)oc2ccccc23)c2cccc3c2c2c(-c4ccccc4)cccc2n3-c2ccccc2)cc1]

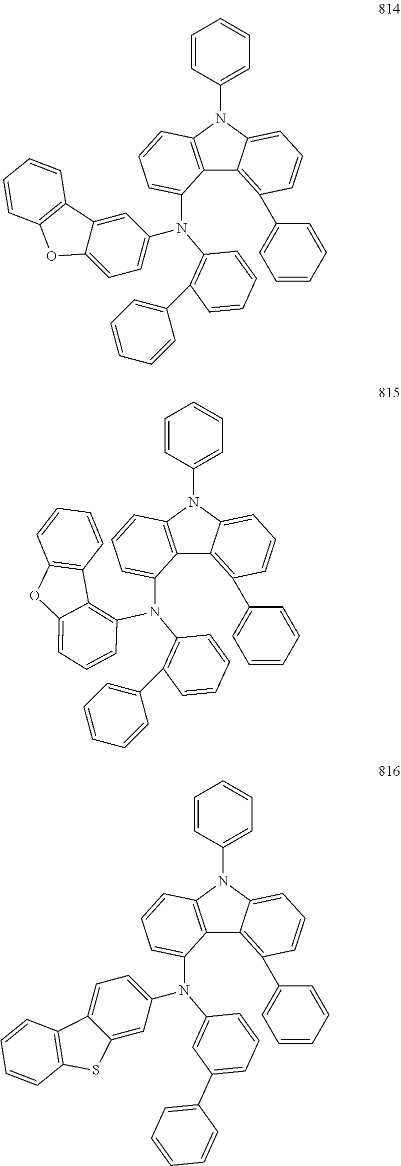 [SMILES: c1ccc(-c2ccccc2N(c2ccc3oc4ccccc4c3c2)c2cccc3c2c2c(-c4ccccc4)cccc2n3-c2ccccc2)cc1 ; c1ccc(-c2ccccc2N(c2cccc3oc4ccccc4c23)c2cccc3c2c2c(-c4ccccc4)cccc2n3-c2ccccc2)cc1 ; c1ccc(-c2cccc(N(c3ccc4c(c3)sc3ccccc34)c3cccc4c3c3c(-c5ccccc5)cccc3n4-c3ccccc3)c2)cc1]

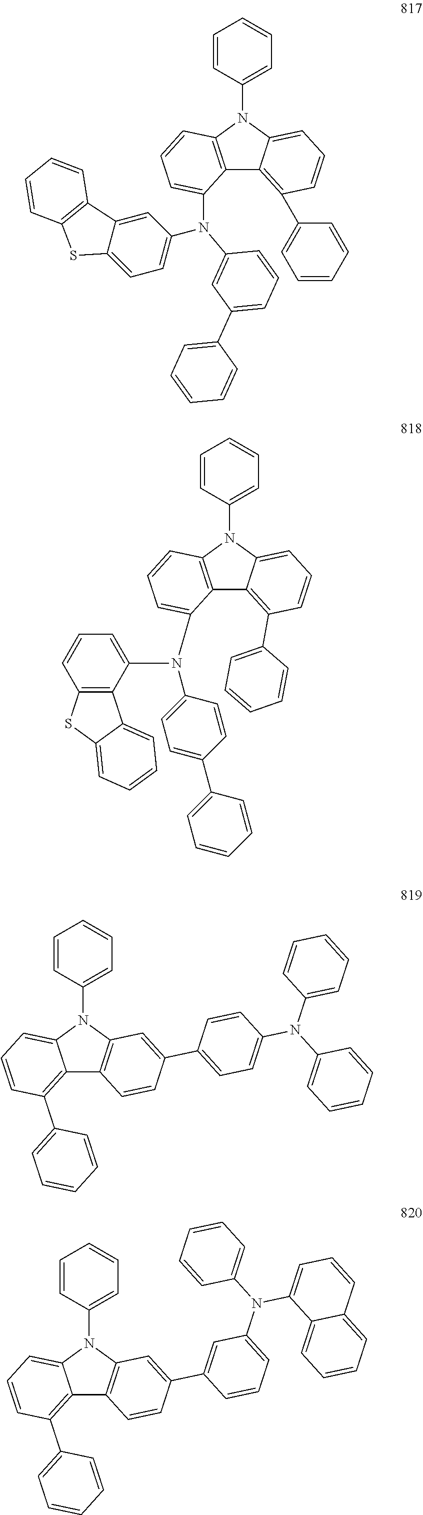

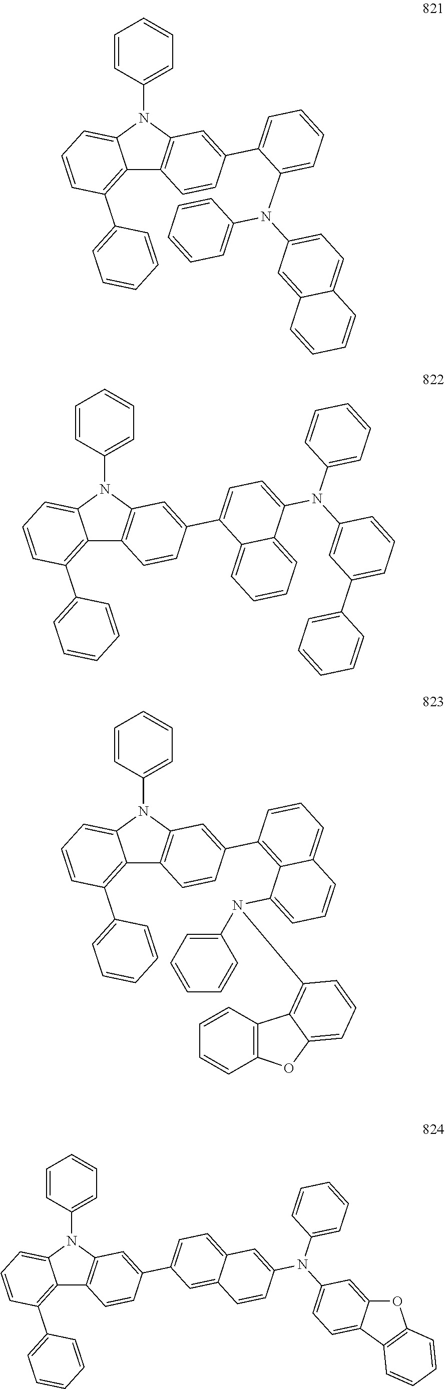 [SMILES: c1ccc(-c2cccc3c2c2ccc(-c4ccccc4N(c4ccccc4)c4ccc5ccccc5c4)cc2n3-c2ccccc2)cc1 ; c1ccc(-c2cccc(N(c3ccccc3)c3ccc(-c4ccc5c6c(-c7ccccc7)cccc6n(-c6ccccc6)c5c4)c4ccccc34)c2)cc1 ; c1ccc(-c2cccc3c2c2ccc(-c4cccc5cccc(N(c6ccccc6)c6cccc7oc8ccccc8c67)c45)cc2n3-c2ccccc2)cc1 ; c1ccc(-c2cccc3c2c2ccc(-c4ccc5cc(N(c6ccccc6)c6ccc7c(c6)oc6ccccc67)ccc5c4)cc2n3-c2ccccc2)cc1]

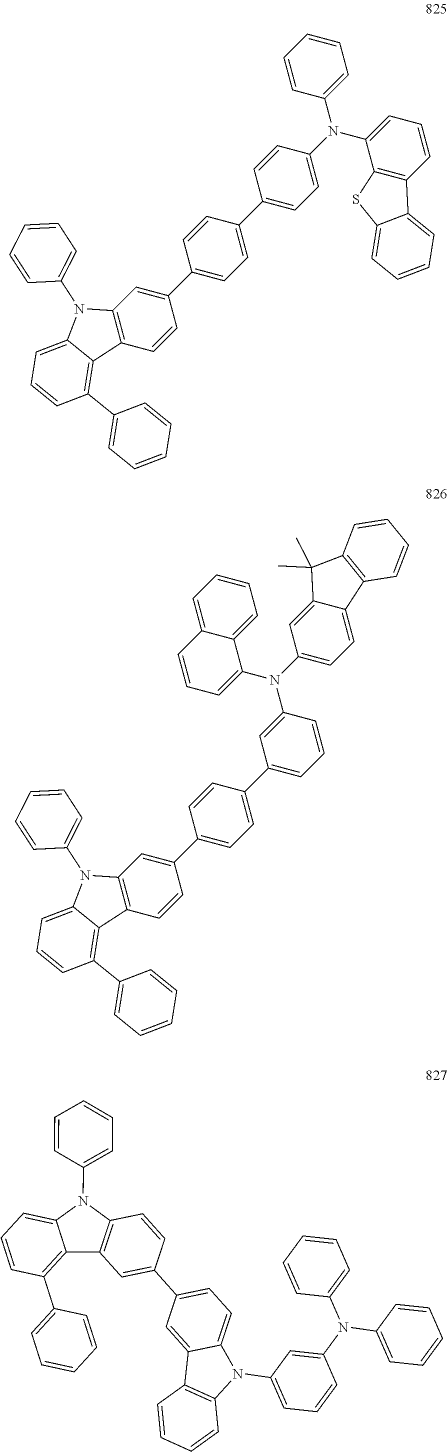 [SMILES: c1ccc(-c2cccc3c2c2ccc(-c4ccc(-c5ccc(N(c6ccccc6)c6cccc7c6sc6ccccc67)cc5)cc4)cc2n3-c2ccccc2)cc1 ; CC1(C)c2ccccc2-c2ccc(N(c3cccc(-c4ccc(-c5ccc6c7c(-c8ccccc8)cccc7n(-c7ccccc7)c6c5)cc4)c3)c3cccc4ccccc34)cc21 ; c1ccc(-c2cccc3c2c2cc(-c4ccc5c(c4)c4ccccc4n5-c4cccc(N(c5ccccc5)c5ccccc5)c4)ccc2n3-c2ccccc2)cc1]

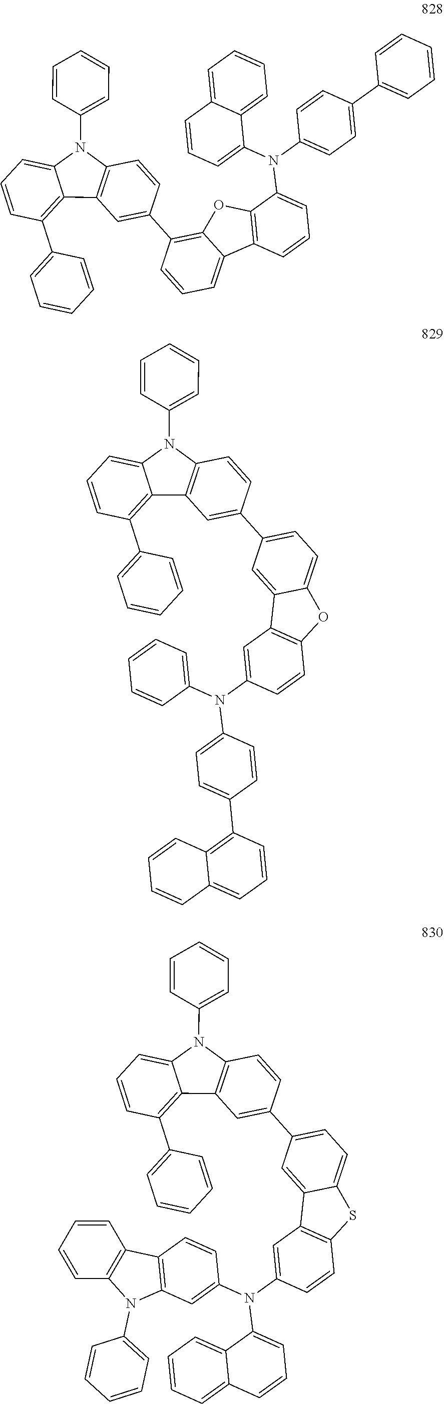

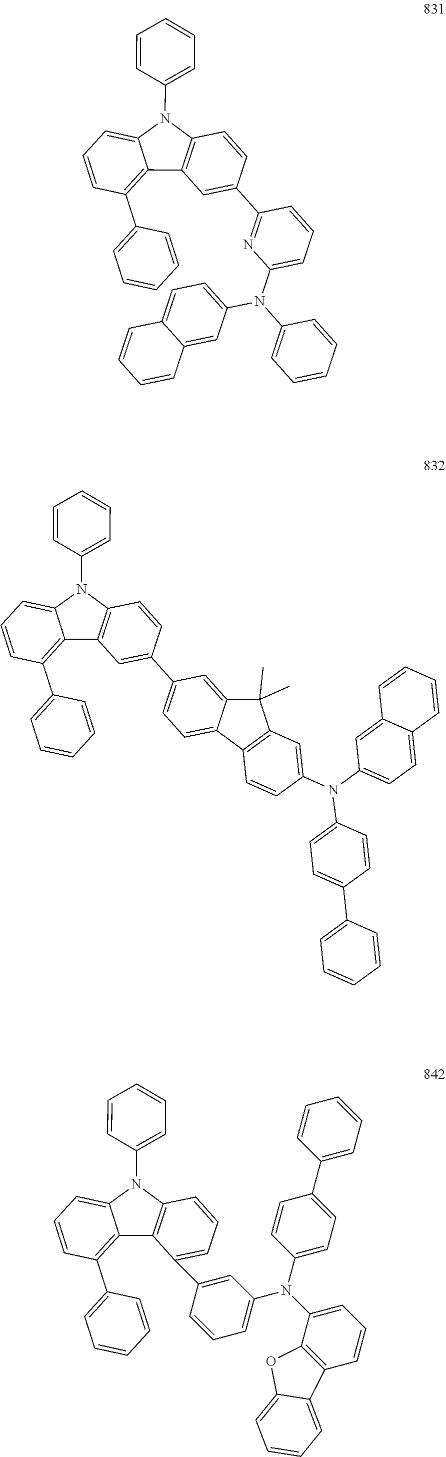 [SMILES: c1ccc(-c2cccc3c2c2cc(-c4cccc(N(c5ccccc5)c5ccc6ccccc6c5)n4)ccc2n3-c2ccccc2)cc1 ; CC1(C)c2cc(-c3ccc4c(c3)c3c(-c5ccccc5)cccc3n4-c3ccccc3)ccc2-c2ccc(N(c3ccc(-c4ccccc4)cc3)c3ccc4ccccc4c3)cc21 ; c1ccc(-c2ccc(N(c3cccc(-c4cccc5c4c4c(-c6ccccc6)cccc4n5-c4ccccc4)c3)c3cccc4c3oc3ccccc34)cc2)cc1]

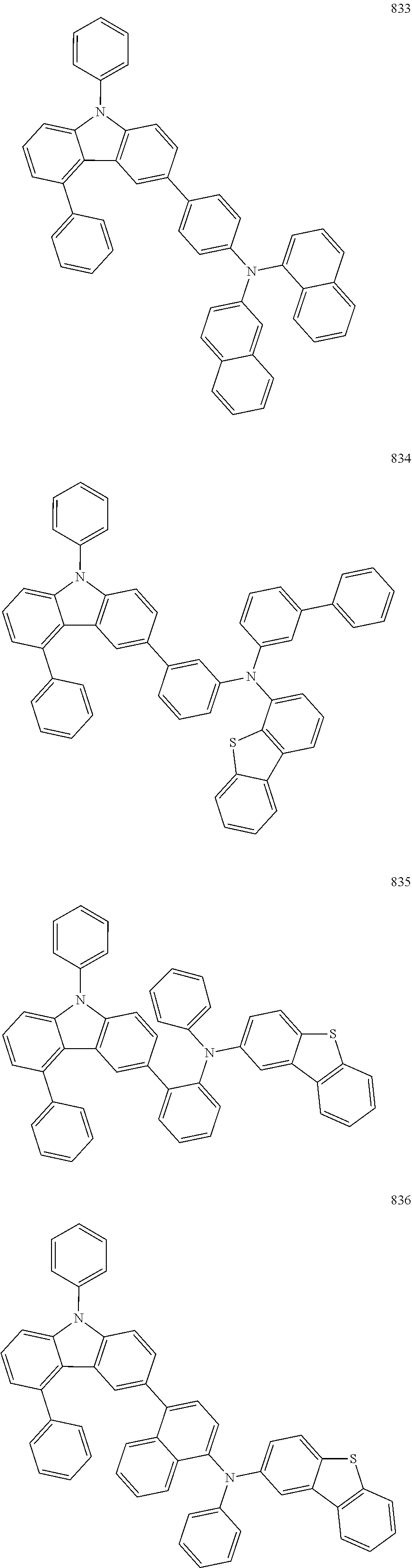 [SMILES: c1ccc(-c2cccc3c2c2cc(-c4ccc(N(c5ccc6ccccc6c5)c5cccc6ccccc56)cc4)ccc2n3-c2ccccc2)cc1 ; c1ccc(-c2cccc(N(c3cccc(-c4ccc5c(c4)c4c(-c6ccccc6)cccc4n5-c4ccccc4)c3)c3cccc4c3sc3ccccc34)c2)cc1 ; c1ccc(-c2cccc3c2c2cc(-c4ccccc4N(c4ccccc4)c4ccc5sc6ccccc6c5c4)ccc2n3-c2ccccc2)cc1 ; c1ccc(-c2cccc3c2c2cc(-c4ccc(N(c5ccccc5)c5ccc6sc7ccccc7c6c5)c5ccccc45)ccc2n3-c2ccccc2)cc1]

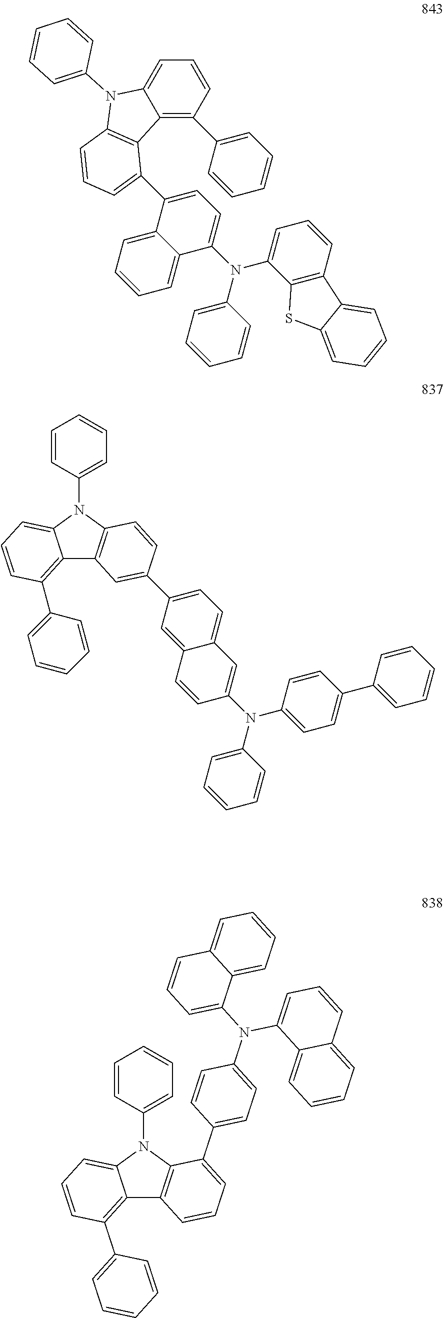 [SMILES: c1ccc(-c2cccc3c2c2c(-c4ccc(N(c5ccccc5)c5cccc6c5sc5ccccc56)c5ccccc45)cccc2n3-c2ccccc2)cc1 ; c1ccc(-c2ccc(N(c3ccccc3)c3ccc4cc(-c5ccc6c(c5)c5c(-c7ccccc7)cccc5n6-c5ccccc5)ccc4c3)cc2)cc1 ; c1ccc(-c2cccc3c2c2cccc(-c4ccc(N(c5cccc6ccccc56)c5cccc6ccccc56)cc4)c2n3-c2ccccc2)cc1]

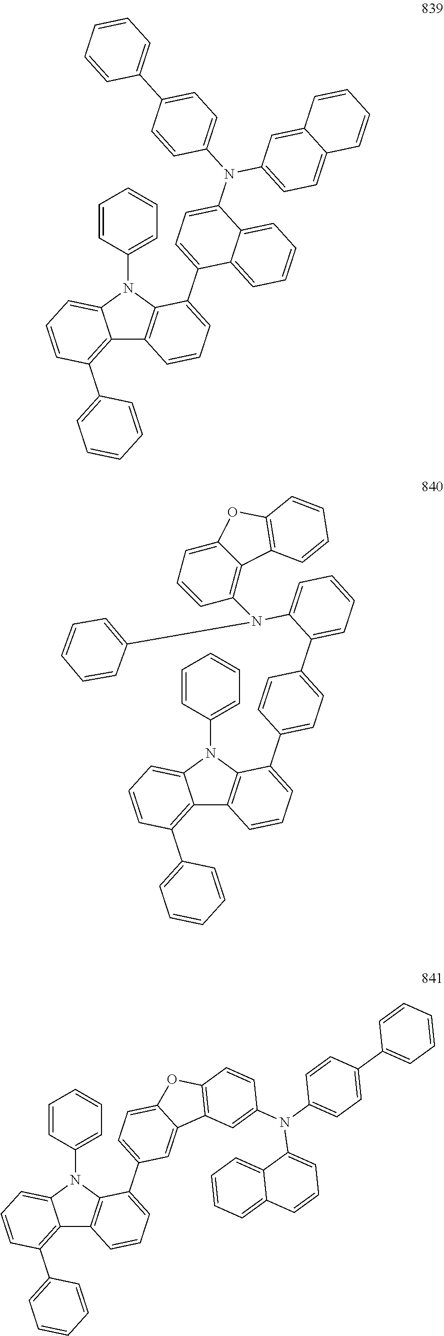 [SMILES: c1ccc(-c2ccc(N(c3ccc4ccccc4c3)c3ccc(-c4cccc5c6c(-c7ccccc7)cccc6n(-c6ccccc6)c45)c4ccccc34)cc2)cc1 ; c1ccc(-c2cccc3c2c2cccc(-c4ccc(-c5ccccc5N(c5ccccc5)c5cccc6oc7ccccc7c56)cc4)c2n3-c2ccccc2)cc1 ; c1ccc(-c2ccc(N(c3ccc4oc5ccc(-c6cccc7c8c(-c9ccccc9)cccc8n(-c8ccccc8)c67)cc5c4c3)c3cccc4ccccc34)cc2)cc1]

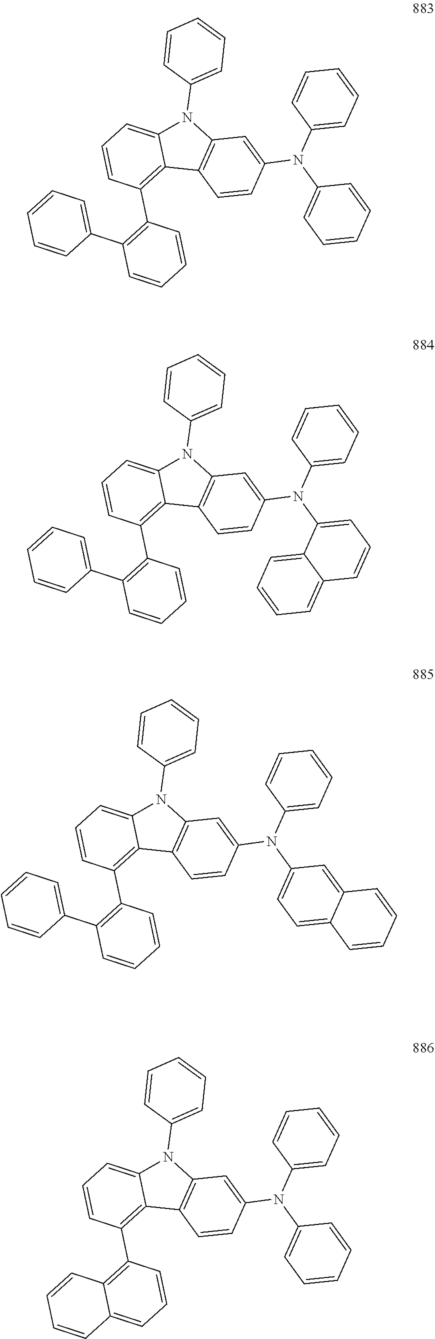 [SMILES: c1ccc(-c2ccccc2-c2cccc3c2c2ccc(N(c4ccccc4)c4ccccc4)cc2n3-c2ccccc2)cc1 ; c1ccc(-c2ccccc2-c2cccc3c2c2ccc(N(c4ccccc4)c4cccc5ccccc45)cc2n3-c2ccccc2)cc1 ; c1ccc(-c2ccccc2-c2cccc3c2c2ccc(N(c4ccccc4)c4ccc5ccccc5c4)cc2n3-c2ccccc2)cc1 ; c1ccc(N(c2ccccc2)c2ccc3c4c(-c5cccc6ccccc56)cccc4n(-c4ccccc4)c3c2)cc1]

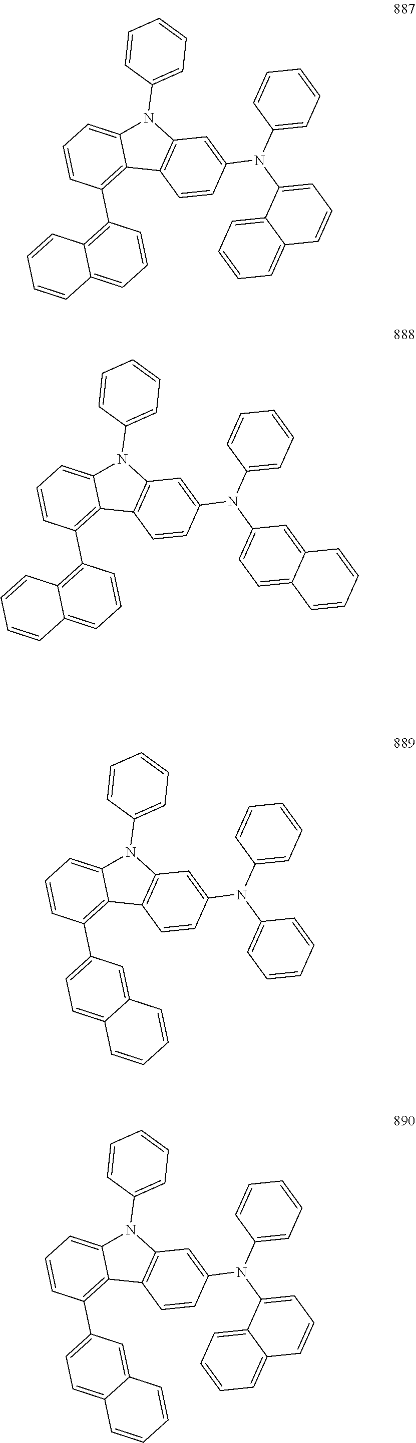 [SMILES: c1ccc(N(c2ccc3c4c(-c5cccc6ccccc56)cccc4n(-c4ccccc4)c3c2)c2cccc3ccccc23)cc1 ; c1ccc(N(c2ccc3ccccc3c2)c2ccc3c4c(-c5cccc6ccccc56)cccc4n(-c4ccccc4)c3c2)cc1 ; c1ccc(N(c2ccccc2)c2ccc3c4c(-c5ccc6ccccc6c5)cccc4n(-c4ccccc4)c3c2)cc1 ; c1ccc(N(c2ccc3c4c(-c5ccc6ccccc6c5)cccc4n(-c4ccccc4)c3c2)c2cccc3ccccc23)cc1]

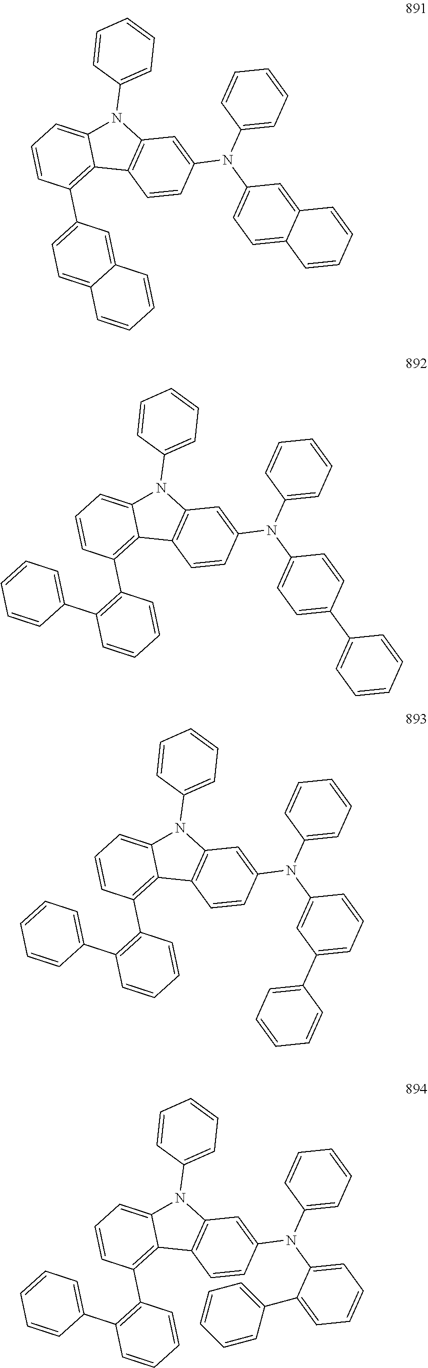 [SMILES: c1ccc(N(c2ccc3ccccc3c2)c2ccc3c4c(-c5ccc6ccccc6c5)cccc4n(-c4ccccc4)c3c2)cc1 ; c1ccc(-c2ccc(N(c3ccccc3)c3ccc4c5c(-c6ccccc6-c6ccccc6)cccc5n(-c5ccccc5)c4c3)cc2)cc1 ; c1ccc(-c2cccc(N(c3ccccc3)c3ccc4c5c(-c6ccccc6-c6ccccc6)cccc5n(-c5ccccc5)c4c3)c2)cc1 ; c1ccc(-c2ccccc2-c2cccc3c2c2ccc(N(c4ccccc4)c4ccccc4-c4ccccc4)cc2n3-c2ccccc2)cc1]

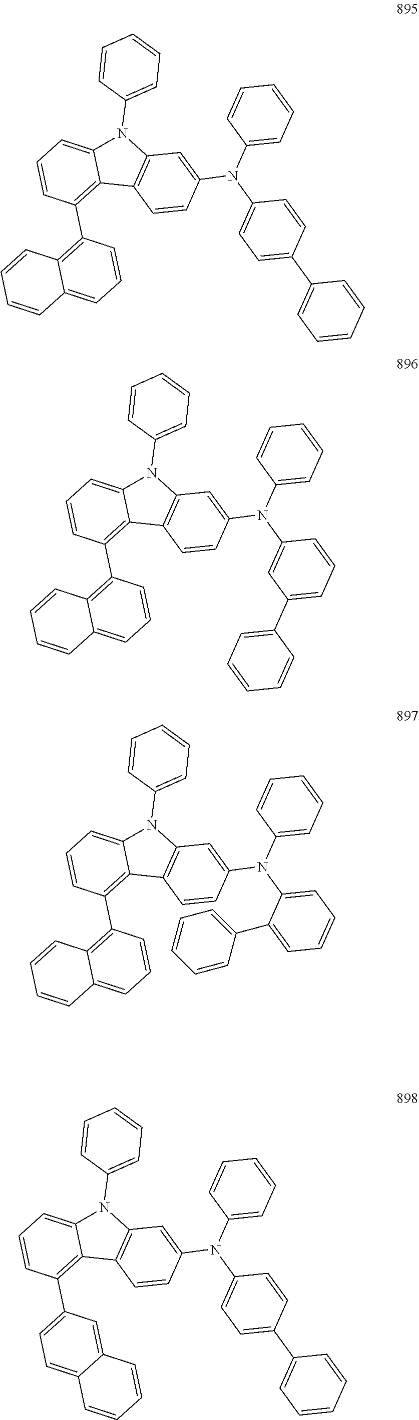 [SMILES: c1ccc(-c2ccc(N(c3ccccc3)c3ccc4c5c(-c6cccc7ccccc67)cccc5n(-c5ccccc5)c4c3)cc2)cc1 ; c1ccc(-c2cccc(N(c3ccccc3)c3ccc4c5c(-c6cccc7ccccc67)cccc5n(-c5ccccc5)c4c3)c2)cc1 ; c1ccc(-c2ccccc2N(c2ccccc2)c2ccc3c4c(-c5cccc6ccccc56)cccc4n(-c4ccccc4)c3c2)cc1 ; c1ccc(-c2ccc(N(c3ccccc3)c3ccc4c5c(-c6ccc7ccccc7c6)cccc5n(-c5ccccc5)c4c3)cc2)cc1]

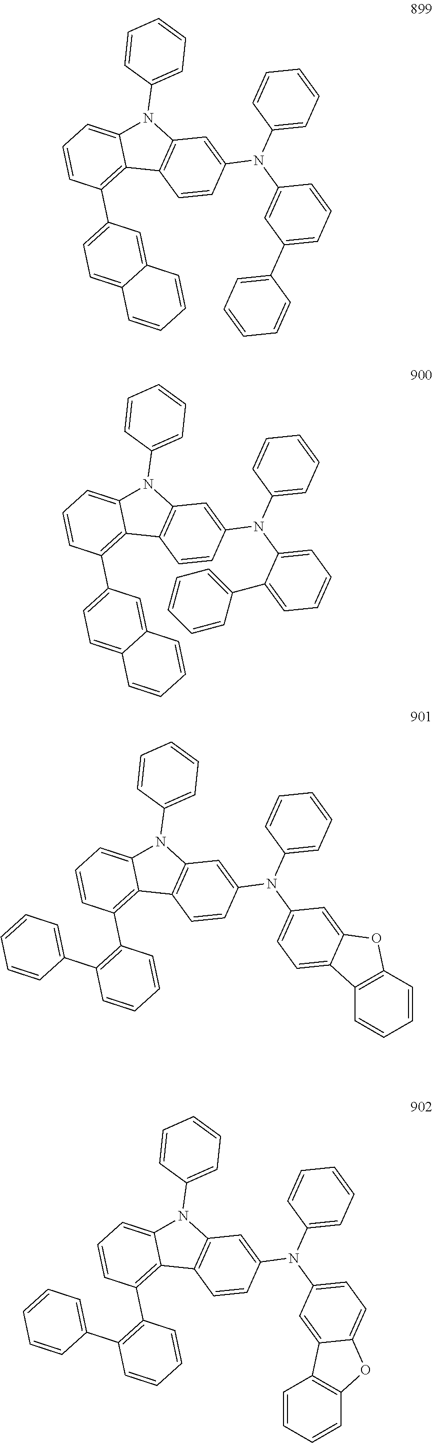 [SMILES: c1ccc(-c2cccc(N(c3ccccc3)c3ccc4c5c(-c6ccc7ccccc7c6)cccc5n(-c5ccccc5)c4c3)c2)cc1 ; c1ccc(-c2ccccc2N(c2ccccc2)c2ccc3c4c(-c5ccc6ccccc6c5)cccc4n(-c4ccccc4)c3c2)cc1 ; c1ccc(-c2ccccc2-c2cccc3c2c2ccc(N(c4ccccc4)c4ccc5c(c4)oc4ccccc45)cc2n3-c2ccccc2)cc1 ; c1ccc(-c2ccccc2-c2cccc3c2c2ccc(N(c4ccccc4)c4ccc5oc6ccccc6c5c4)cc2n3-c2ccccc2)cc1]

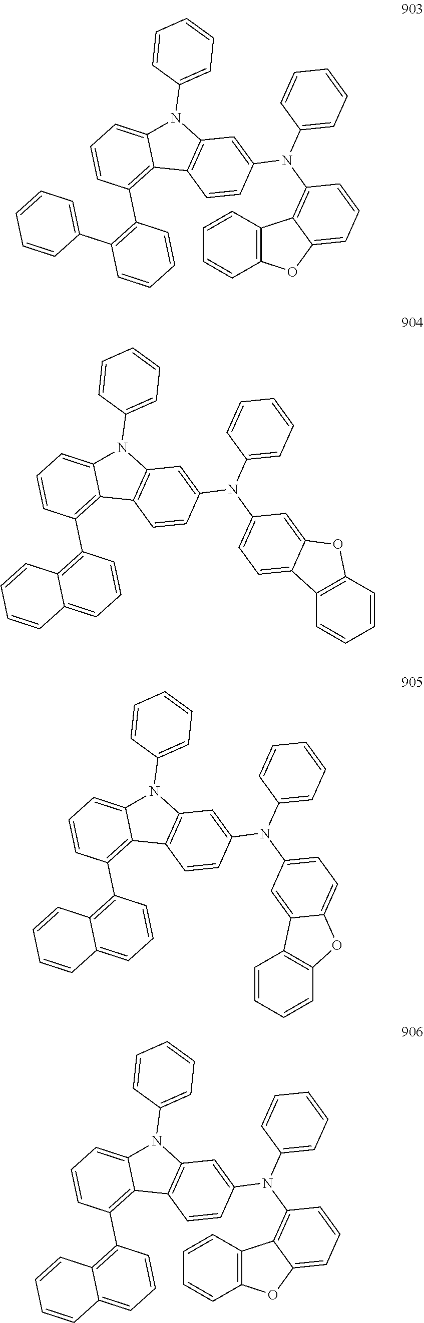 [SMILES: c1ccc(-c2ccccc2-c2cccc3c2c2ccc(N(c4ccccc4)c4cccc5oc6ccccc6c45)cc2n3-c2ccccc2)cc1 ; c1ccc(N(c2ccc3c(c2)oc2ccccc23)c2ccc3c4c(-c5cccc6ccccc56)cccc4n(-c4ccccc4)c3c2)cc1 ; c1ccc(N(c2ccc3oc4ccccc4c3c2)c2ccc3c4c(-c5cccc6ccccc56)cccc4n(-c4ccccc4)c3c2)cc1 ; c1ccc(N(c2ccc3c4c(-c5cccc6ccccc56)cccc4n(-c4ccccc4)c3c2)c2cccc3oc4ccccc4c23)cc1]

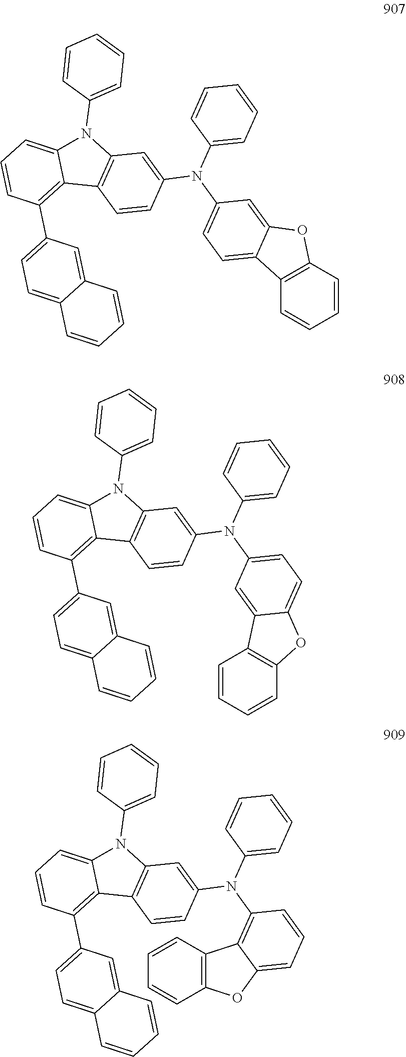

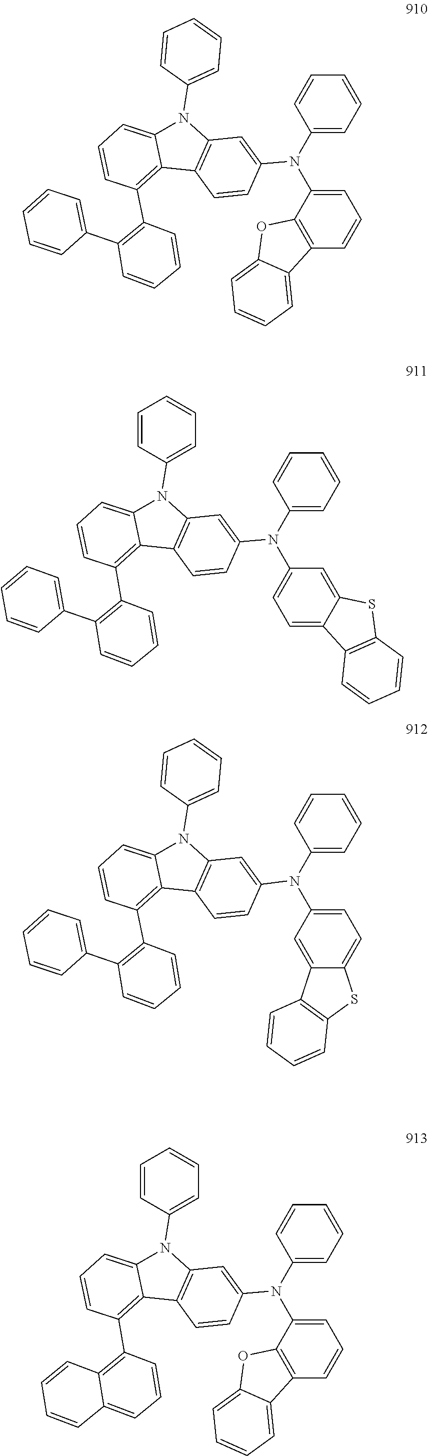 [SMILES: c1ccc(-c2ccccc2-c2cccc3c2c2ccc(N(c4ccccc4)c4cccc5c4oc4ccccc45)cc2n3-c2ccccc2)cc1 ; c1ccc(-c2ccccc2-c2cccc3c2c2ccc(N(c4ccccc4)c4ccc5c(c4)sc4ccccc45)cc2n3-c2ccccc2)cc1 ; c1ccc(-c2ccccc2-c2cccc3c2c2ccc(N(c4ccccc4)c4ccc5sc6ccccc6c5c4)cc2n3-c2ccccc2)cc1 ; c1ccc(N(c2ccc3c4c(-c5cccc6ccccc56)cccc4n(-c4ccccc4)c3c2)c2cccc3c2oc2ccccc23)cc1]

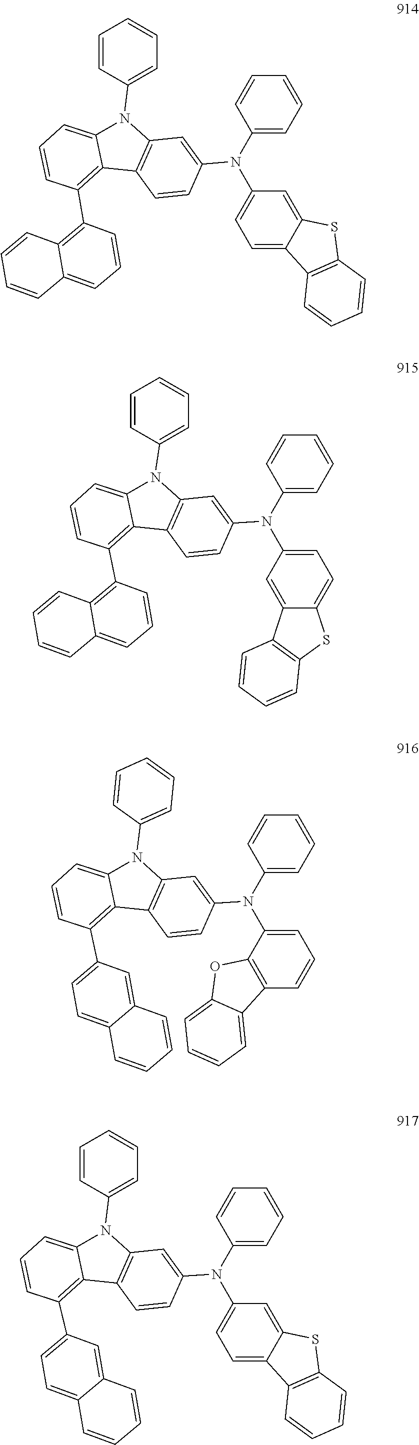 [SMILES: c1ccc(N(c2ccc3c(c2)sc2ccccc23)c2ccc3c4c(-c5cccc6ccccc56)cccc4n(-c4ccccc4)c3c2)cc1 ; c1ccc(N(c2ccc3sc4ccccc4c3c2)c2ccc3c4c(-c5cccc6ccccc56)cccc4n(-c4ccccc4)c3c2)cc1 ; c1ccc(N(c2ccc3c4c(-c5ccc6ccccc6c5)cccc4n(-c4ccccc4)c3c2)c2cccc3c2oc2ccccc23)cc1 ; c1ccc(N(c2ccc3c(c2)sc2ccccc23)c2ccc3c4c(-c5ccc6ccccc6c5)cccc4n(-c4ccccc4)c3c2)cc1]

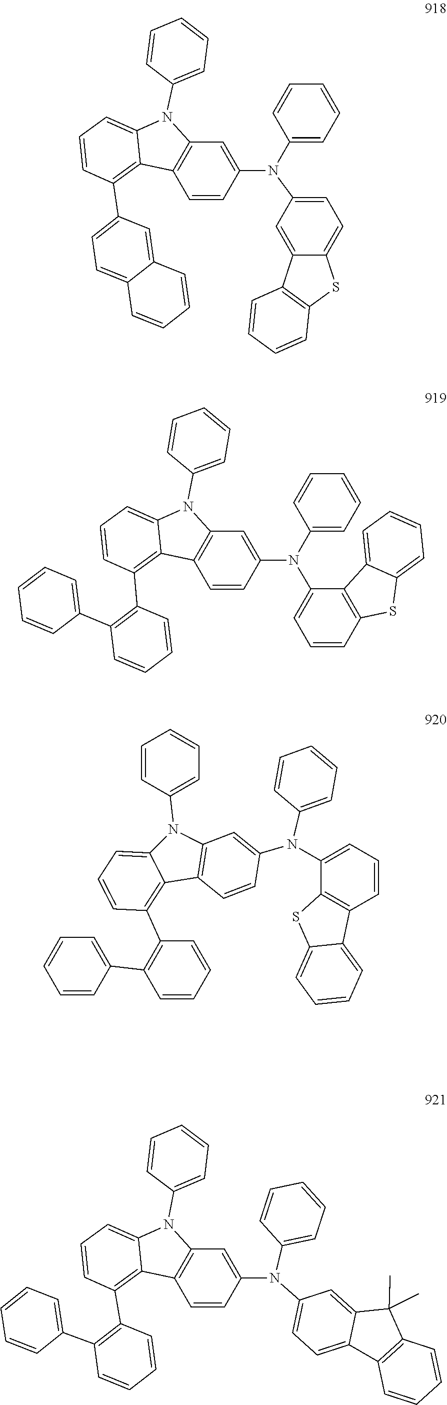 [SMILES: c1ccc(N(c2ccc3sc4ccccc4c3c2)c2ccc3c4c(-c5ccc6ccccc6c5)cccc4n(-c4ccccc4)c3c2)cc1 ; c1ccc(-c2ccccc2-c2cccc3c2c2ccc(N(c4ccccc4)c4cccc5sc6ccccc6c45)cc2n3-c2ccccc2)cc1 ; c1ccc(-c2ccccc2-c2cccc3c2c2ccc(N(c4ccccc4)c4cccc5c4sc4ccccc45)cc2n3-c2ccccc2)cc1 ; CC1(C)c2ccccc2-c2ccc(N(c3ccccc3)c3ccc4c5c(-c6ccccc6-c6ccccc6)cccc5n(-c5ccccc5)c4c3)cc21]

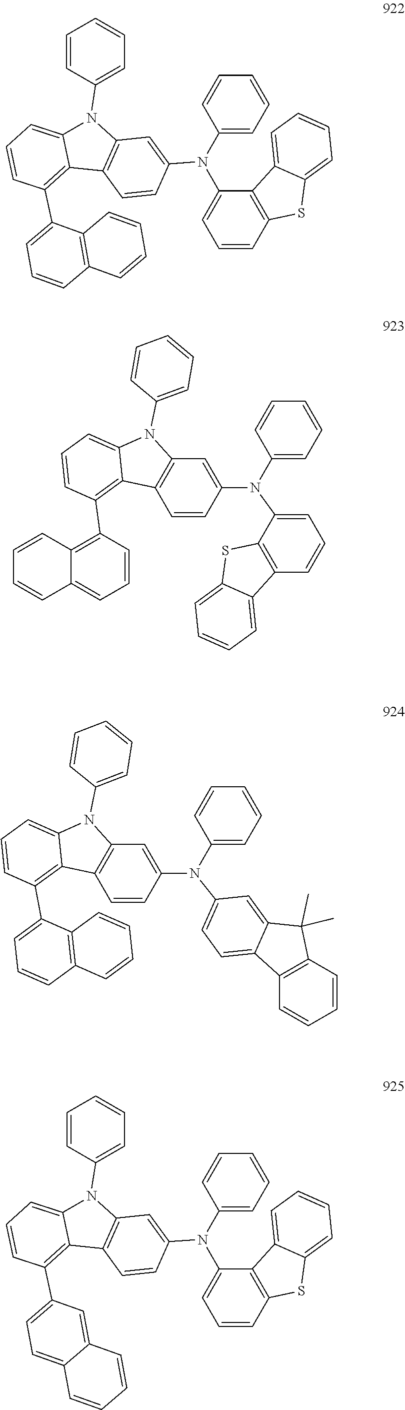 [SMILES: c1ccc(N(c2ccc3c4c(-c5cccc6ccccc56)cccc4n(-c4ccccc4)c3c2)c2cccc3sc4ccccc4c23)cc1 ; c1ccc(N(c2ccc3c4c(-c5cccc6ccccc56)cccc4n(-c4ccccc4)c3c2)c2cccc3c2sc2ccccc23)cc1 ; CC1(C)c2ccccc2-c2ccc(N(c3ccccc3)c3ccc4c5c(-c6cccc7ccccc67)cccc5n(-c5ccccc5)c4c3)cc21 ; c1ccc(N(c2ccc3c4c(-c5ccc6ccccc6c5)cccc4n(-c4ccccc4)c3c2)c2cccc3sc4ccccc4c23)cc1]

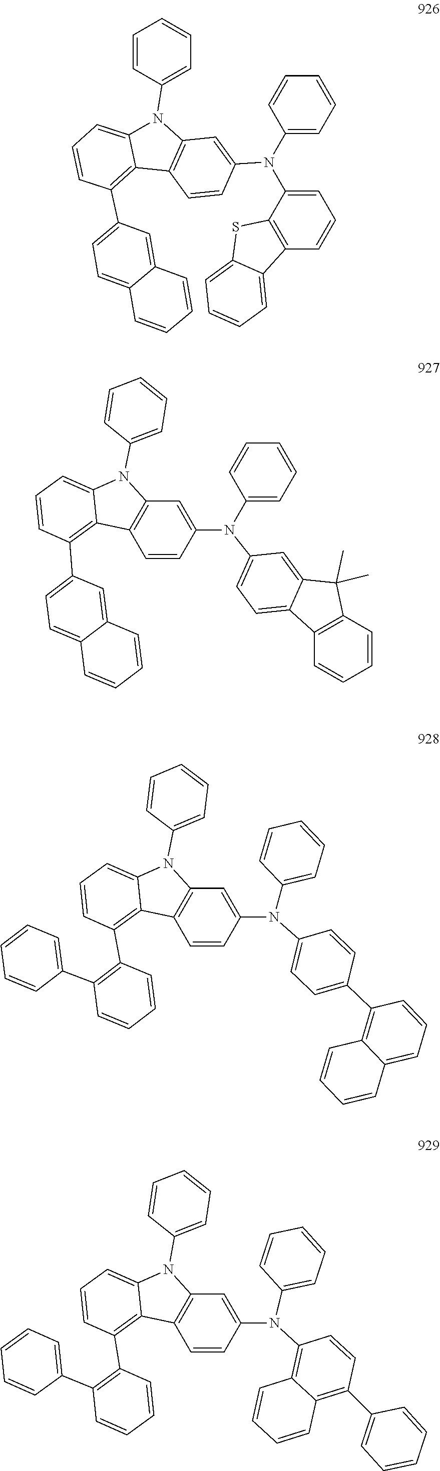 [SMILES: c1ccc(N(c2ccc3c4c(-c5ccc6ccccc6c5)cccc4n(-c4ccccc4)c3c2)c2cccc3c2sc2ccccc23)cc1 ; CC1(C)c2ccccc2-c2ccc(N(c3ccccc3)c3ccc4c5c(-c6ccc7ccccc7c6)cccc5n(-c5ccccc5)c4c3)cc21 ; c1ccc(-c2ccccc2-c2cccc3c2c2ccc(N(c4ccccc4)c4ccc(-c5cccc6ccccc56)cc4)cc2n3-c2ccccc2)cc1 ; c1ccc(-c2ccccc2-c2cccc3c2c2ccc(N(c4ccccc4)c4ccc(-c5ccccc5)c5ccccc45)cc2n3-c2ccccc2)cc1]

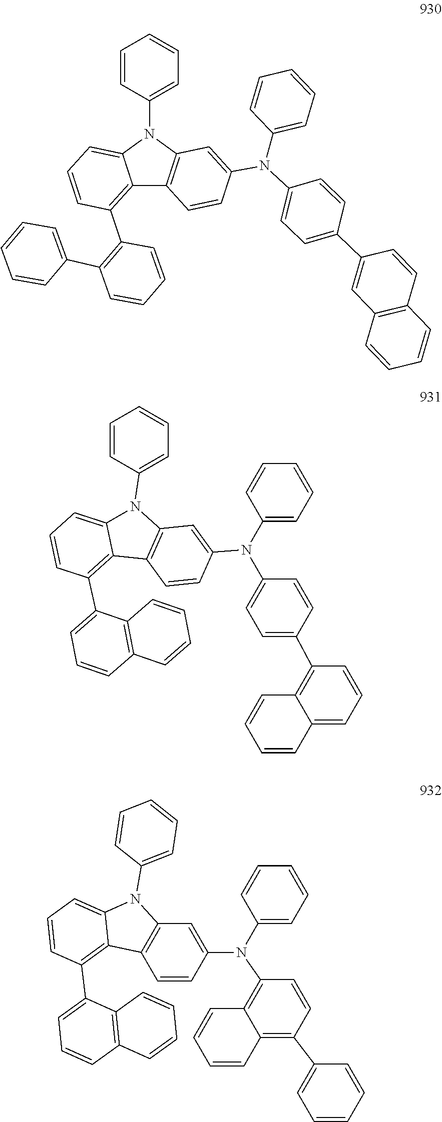 [SMILES: c1ccc(-c2ccccc2-c2cccc3c2c2ccc(N(c4ccccc4)c4ccc(-c5ccc6ccccc6c5)cc4)cc2n3-c2ccccc2)cc1 ; c1ccc(N(c2ccc(-c3cccc4ccccc34)cc2)c2ccc3c4c(-c5cccc6ccccc56)cccc4n(-c4ccccc4)c3c2)cc1 ; c1ccc(-c2ccc(N(c3ccccc3)c3ccc4c5c(-c6cccc7ccccc67)cccc5n(-c5ccccc5)c4c3)c3ccccc23)cc1]

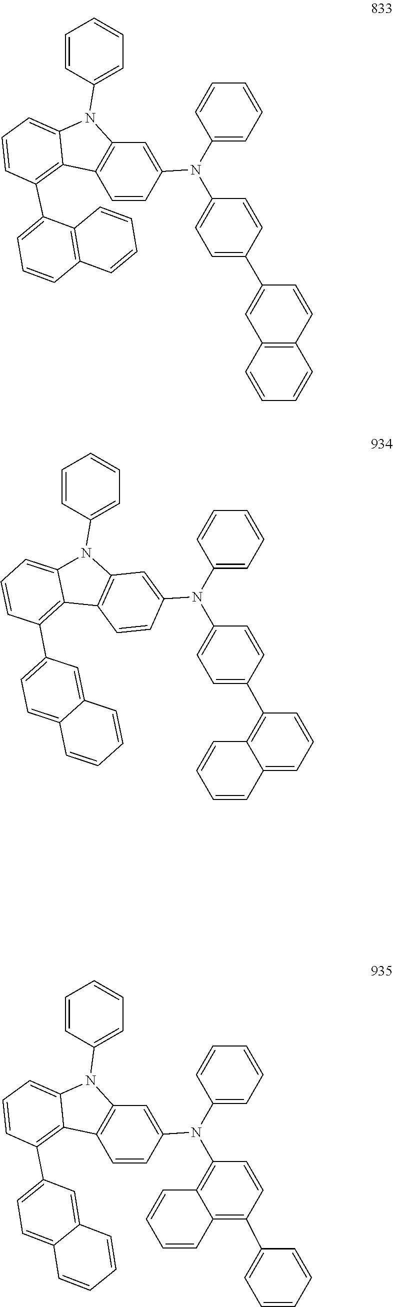 [SMILES: c1ccc(N(c2ccc(-c3ccc4ccccc4c3)cc2)c2ccc3c4c(-c5cccc6ccccc56)cccc4n(-c4ccccc4)c3c2)cc1 ; c1ccc(N(c2ccc(-c3cccc4ccccc34)cc2)c2ccc3c4c(-c5ccc6ccccc6c5)cccc4n(-c4ccccc4)c3c2)cc1 ; c1ccc(-c2ccc(N(c3ccccc3)c3ccc4c5c(-c6ccc7ccccc7c6)cccc5n(-c5ccccc5)c4c3)c3ccccc23)cc1]

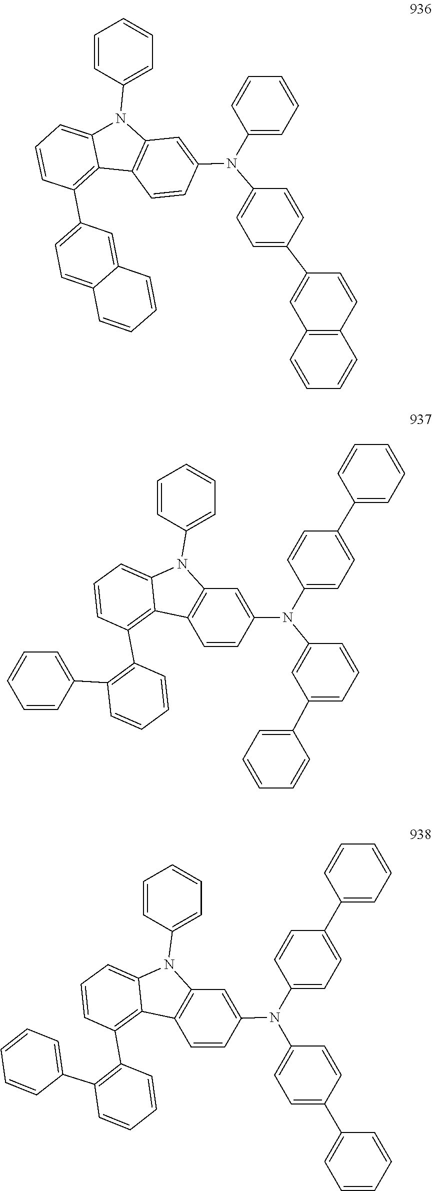 [SMILES: c1ccc(N(c2ccc(-c3ccc4ccccc4c3)cc2)c2ccc3c4c(-c5ccc6ccccc6c5)cccc4n(-c4ccccc4)c3c2)cc1 ; c1ccc(-c2ccc(N(c3cccc(-c4ccccc4)c3)c3ccc4c5c(-c6ccccc6-c6ccccc6)cccc5n(-c5ccccc5)c4c3)cc2)cc1 ; c1ccc(-c2ccc(N(c3ccc(-c4ccccc4)cc3)c3ccc4c5c(-c6ccccc6-c6ccccc6)cccc5n(-c5ccccc5)c4c3)cc2)cc1]

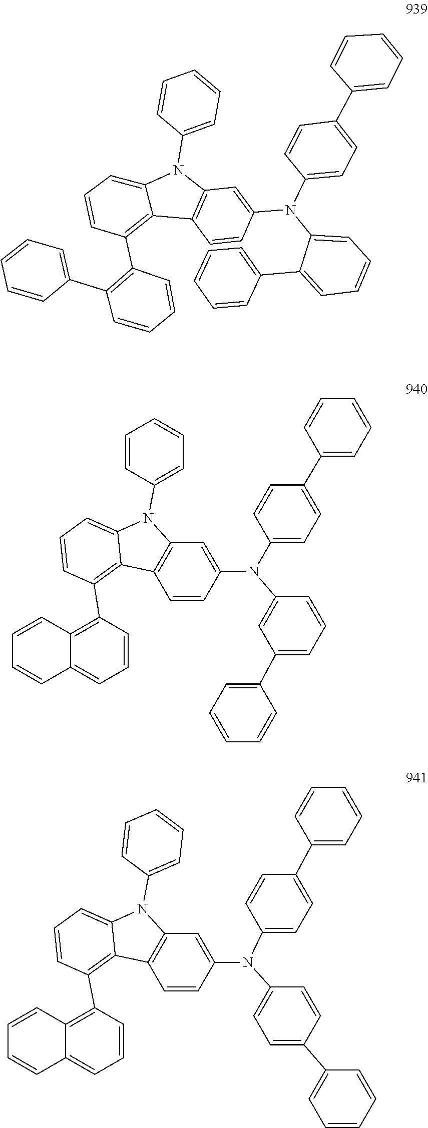 [SMILES: c1ccc(-c2ccc(N(c3ccc4c5c(-c6ccccc6-c6ccccc6)cccc5n(-c5ccccc5)c4c3)c3ccccc3-c3ccccc3)cc2)cc1 ; c1ccc(-c2ccc(N(c3cccc(-c4ccccc4)c3)c3ccc4c5c(-c6cccc7ccccc67)cccc5n(-c5ccccc5)c4c3)cc2)cc1 ; c1ccc(-c2ccc(N(c3ccc(-c4ccccc4)cc3)c3ccc4c5c(-c6cccc7ccccc67)cccc5n(-c5ccccc5)c4c3)cc2)cc1]

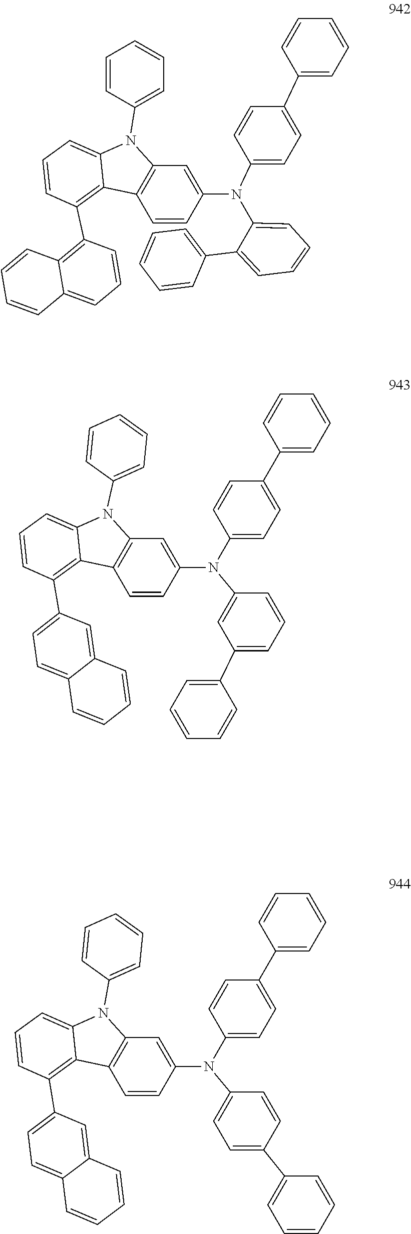 [SMILES: c1ccc(-c2ccc(N(c3ccc4c5c(-c6cccc7ccccc67)cccc5n(-c5ccccc5)c4c3)c3ccccc3-c3ccccc3)cc2)cc1 ; c1ccc(-c2ccc(N(c3cccc(-c4ccccc4)c3)c3ccc4c5c(-c6ccc7ccccc7c6)cccc5n(-c5ccccc5)c4c3)cc2)cc1 ; c1ccc(-c2ccc(N(c3ccc(-c4ccccc4)cc3)c3ccc4c5c(-c6ccc7ccccc7c6)cccc5n(-c5ccccc5)c4c3)cc2)cc1]

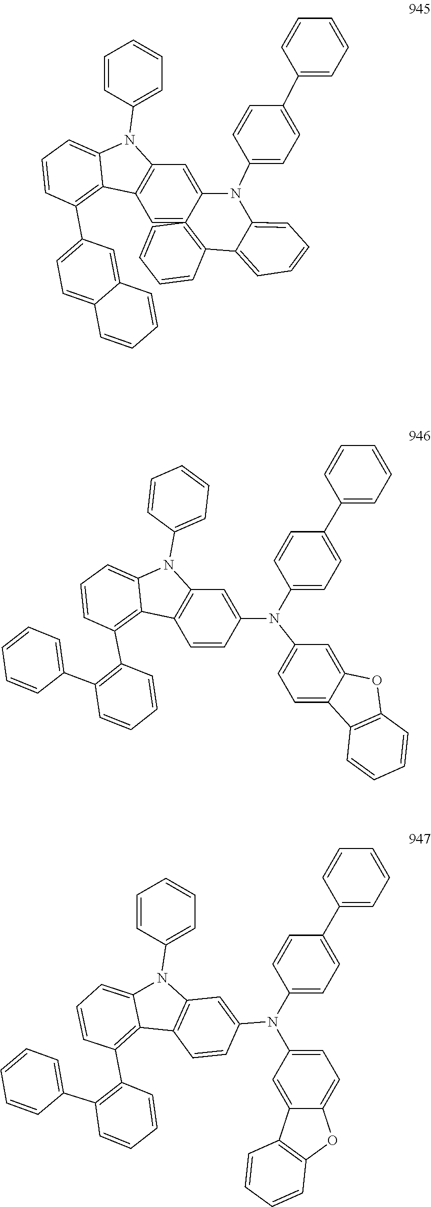

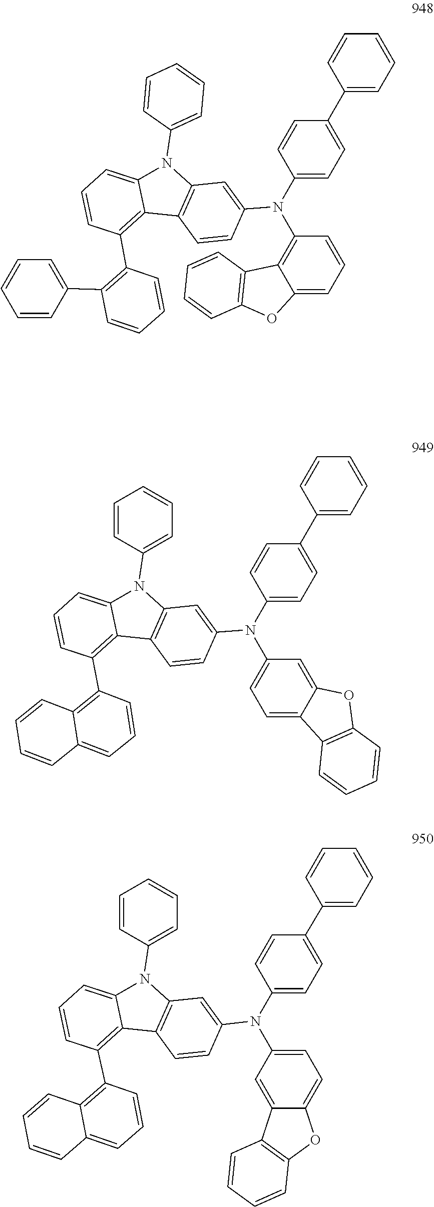 [SMILES: c1ccc(-c2ccc(N(c3ccc4c5c(-c6ccccc6-c6ccccc6)cccc5n(-c5ccccc5)c4c3)c3cccc4oc5ccccc5c34)cc2)cc1 ; c1ccc(-c2ccc(N(c3ccc4c(c3)oc3ccccc34)c3ccc4c5c(-c6cccc7ccccc67)cccc5n(-c5ccccc5)c4c3)cc2)cc1 ; c1ccc(-c2ccc(N(c3ccc4oc5ccccc5c4c3)c3ccc4c5c(-c6cccc7ccccc67)cccc5n(-c5ccccc5)c4c3)cc2)cc1]

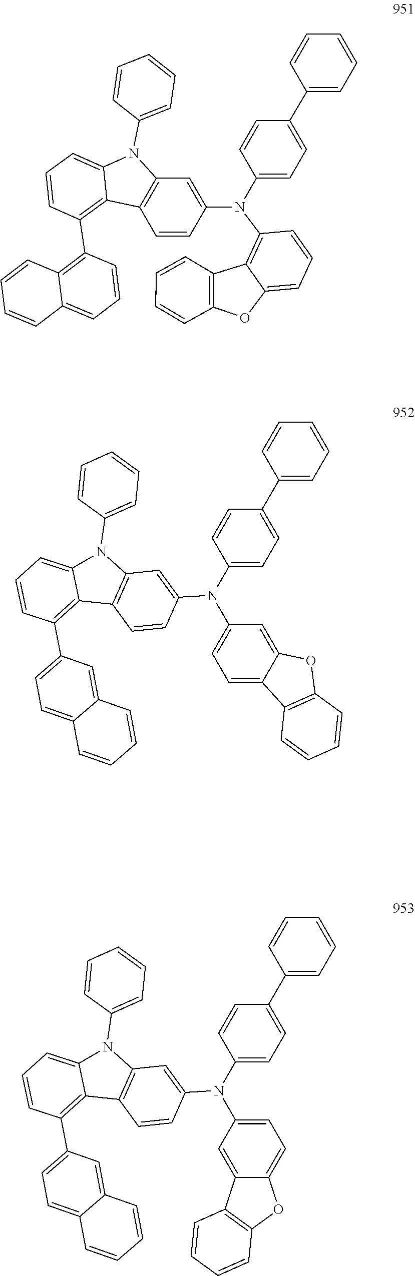 [SMILES: c1ccc(-c2ccc(N(c3ccc4c5c(-c6cccc7ccccc67)cccc5n(-c5ccccc5)c4c3)c3cccc4oc5ccccc5c34)cc2)cc1 ; c1ccc(-c2ccc(N(c3ccc4c(c3)oc3ccccc34)c3ccc4c5c(-c6ccc7ccccc7c6)cccc5n(-c5ccccc5)c4c3)cc2)cc1 ; c1ccc(-c2ccc(N(c3ccc4oc5ccccc5c4c3)c3ccc4c5c(-c6ccc7ccccc7c6)cccc5n(-c5ccccc5)c4c3)cc2)cc1]

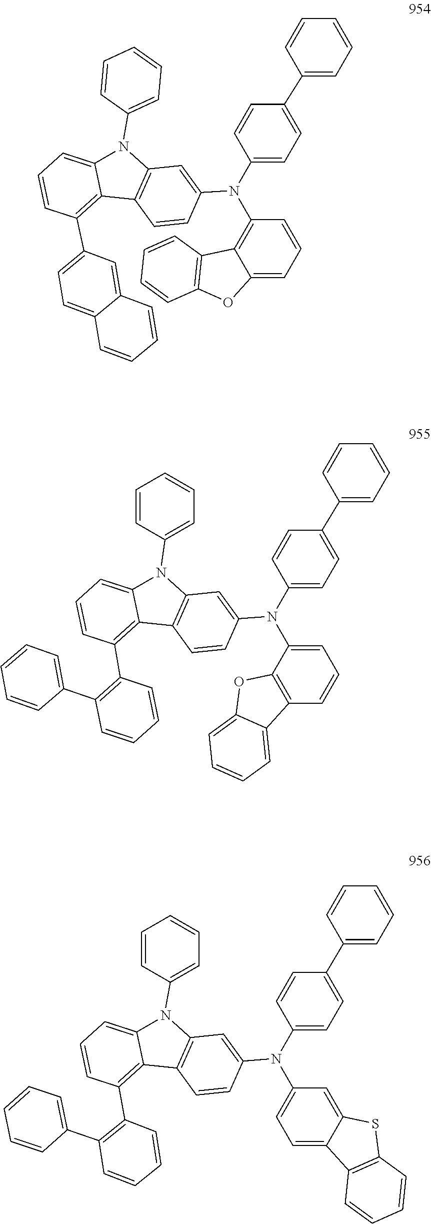 [SMILES: c1ccc(-c2ccc(N(c3ccc4c5c(-c6ccc7ccccc7c6)cccc5n(-c5ccccc5)c4c3)c3cccc4oc5ccccc5c34)cc2)cc1 ; c1ccc(-c2ccc(N(c3ccc4c5c(-c6ccccc6-c6ccccc6)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6ccccc6-c6ccccc6)cccc5n(-c5ccccc5)c4c3)cc2)cc1]

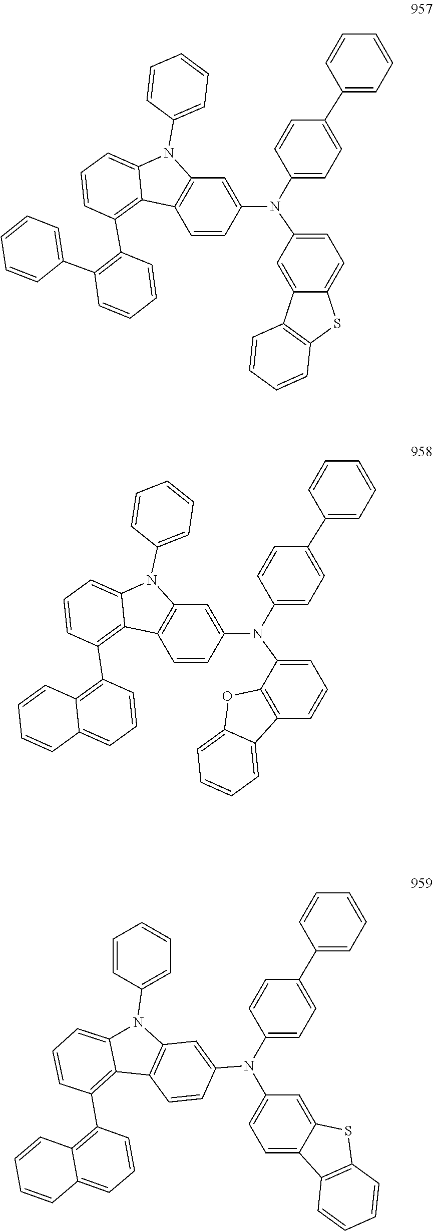 [SMILES: c1ccc(-c2ccc(N(c3ccc4sc5ccccc5c4c3)c3ccc4c5c(-c6ccccc6-c6ccccc6)cccc5n(-c5ccccc5)c4c3)cc2)cc1 ; c1ccc(-c2ccc(N(c3ccc4c5c(-c6cccc7ccccc67)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6cccc7ccccc67)cccc5n(-c5ccccc5)c4c3)cc2)cc1]

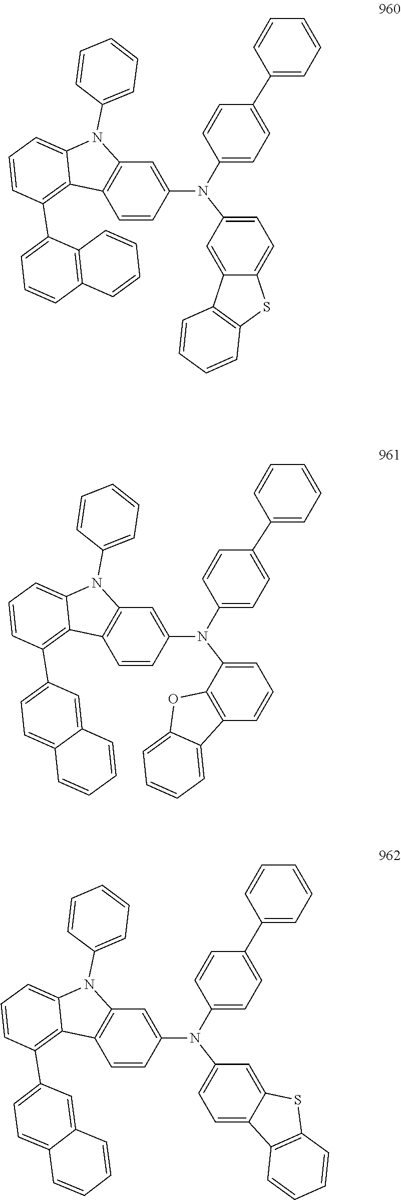 [SMILES: c1ccc(-c2ccc(N(c3ccc4sc5ccccc5c4c3)c3ccc4c5c(-c6cccc7ccccc67)cccc5n(-c5ccccc5)c4c3)cc2)cc1 ; c1ccc(-c2ccc(N(c3ccc4c5c(-c6ccc7ccccc7c6)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6ccc7ccccc7c6)cccc5n(-c5ccccc5)c4c3)cc2)cc1]

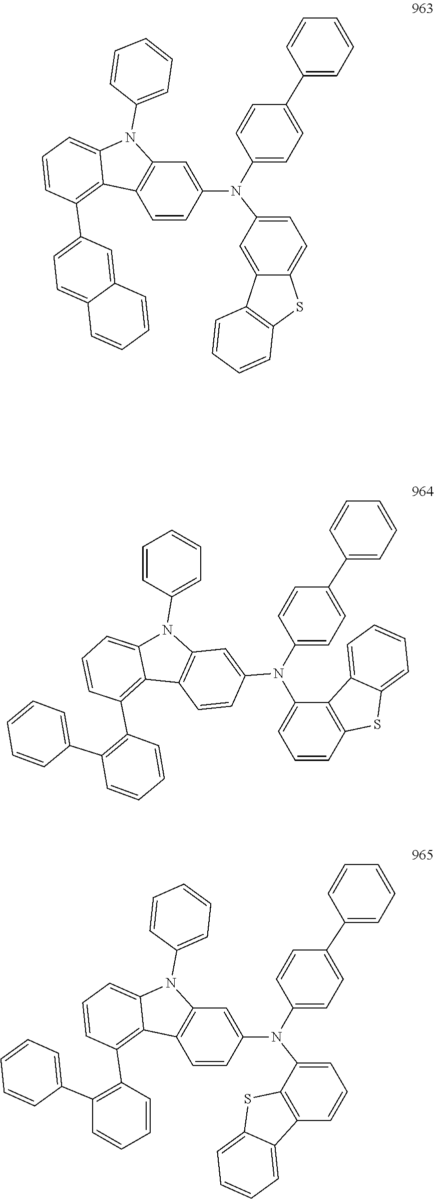 [SMILES: c1ccc(-c2ccc(N(c3ccc4sc5ccccc5c4c3)c3ccc4c5c(-c6ccc7ccccc7c6)cccc5n(-c5ccccc5)c4c3)cc2)cc1 ; c1ccc(-c2ccc(N(c3ccc4c5c(-c6ccccc6-c6ccccc6)cccc5n(-c5ccccc5)c4c3)c3cccc4sc5ccccc5c34)cc2)cc1 ; c1ccc(-c2ccc(N(c3ccc4c5c(-c6ccccc6-c6ccccc6)cccc5n(-c5ccccc5)c4c3)c3cccc4c3sc3ccccc34)cc2)cc1]

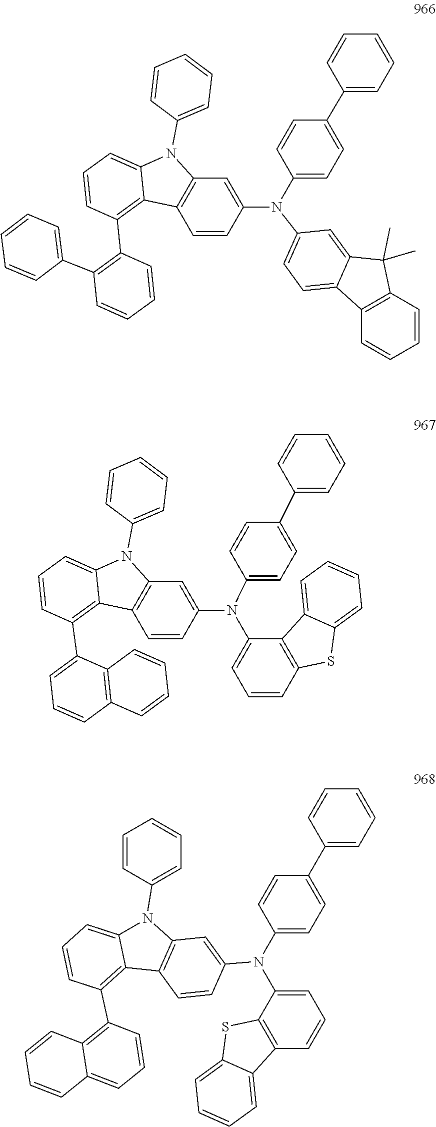 [SMILES: CC1(C)c2ccccc2-c2ccc(N(c3ccc(-c4ccccc4)cc3)c3ccc4c5c(-c6ccccc6-c6ccccc6)cccc5n(-c5ccccc5)c4c3)cc21 ; c1ccc(-c2ccc(N(c3ccc4c5c(-c6cccc7ccccc67)cccc5n(-c5ccccc5)c4c3)c3cccc4sc5ccccc5c34)cc2)cc1 ; c1ccc(-c2ccc(N(c3ccc4c5c(-c6cccc7ccccc67)cccc5n(-c5ccccc5)c4c3)c3cccc4c3sc3ccccc34)cc2)cc1]

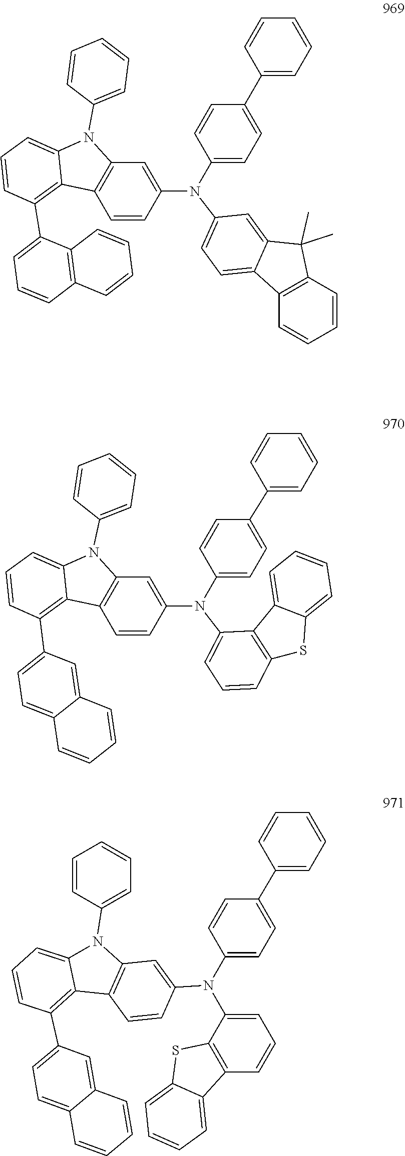 [SMILES: CC1(C)c2ccccc2-c2ccc(N(c3ccc(-c4ccccc4)cc3)c3ccc4c5c(-c6cccc7ccccc67)cccc5n(-c5ccccc5)c4c3)cc21 ; c1ccc(-c2ccc(N(c3ccc4c5c(-c6ccc7ccccc7c6)cccc5n(-c5ccccc5)c4c3)c3cccc4sc5ccccc5c34)cc2)cc1 ; c1ccc(-c2ccc(N(c3ccc4c5c(-c6ccc7ccccc7c6)cccc5n(-c5ccccc5)c4c3)c3cccc4c3sc3ccccc34)cc2)cc1]

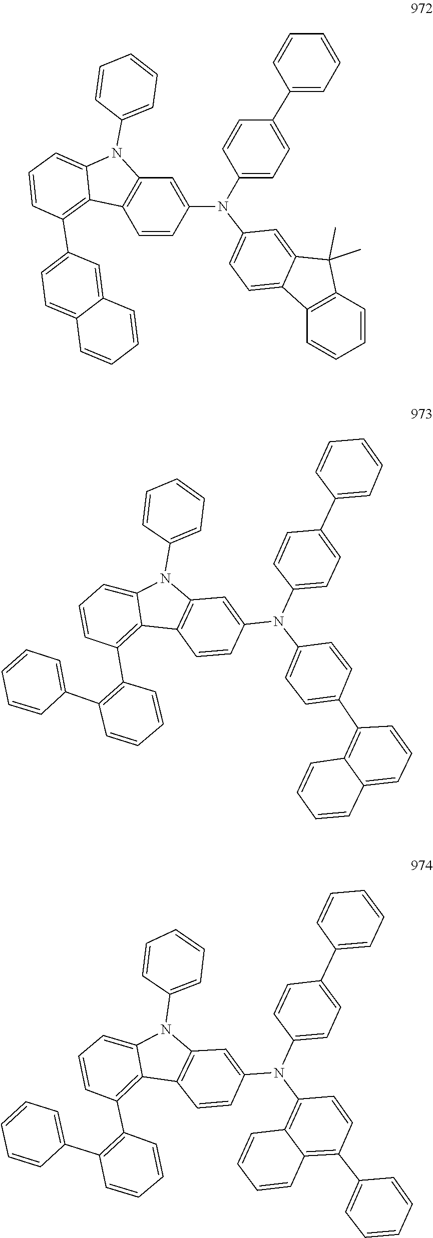 [SMILES: CC1(C)c2ccccc2-c2ccc(N(c3ccc(-c4ccccc4)cc3)c3ccc4c5c(-c6ccc7ccccc7c6)cccc5n(-c5ccccc5)c4c3)cc21 ; c1ccc(-c2ccc(N(c3ccc(-c4cccc5ccccc45)cc3)c3ccc4c5c(-c6ccccc6-c6ccccc6)cccc5n(-c5ccccc5)c4c3)cc2)cc1 ; c1ccc(-c2ccc(N(c3ccc4c5c(-c6ccccc6-c6ccccc6)cccc5n(-c5ccccc5)c4c3)c3ccc(-c4ccccc4)c4ccccc34)cc2)cc1]

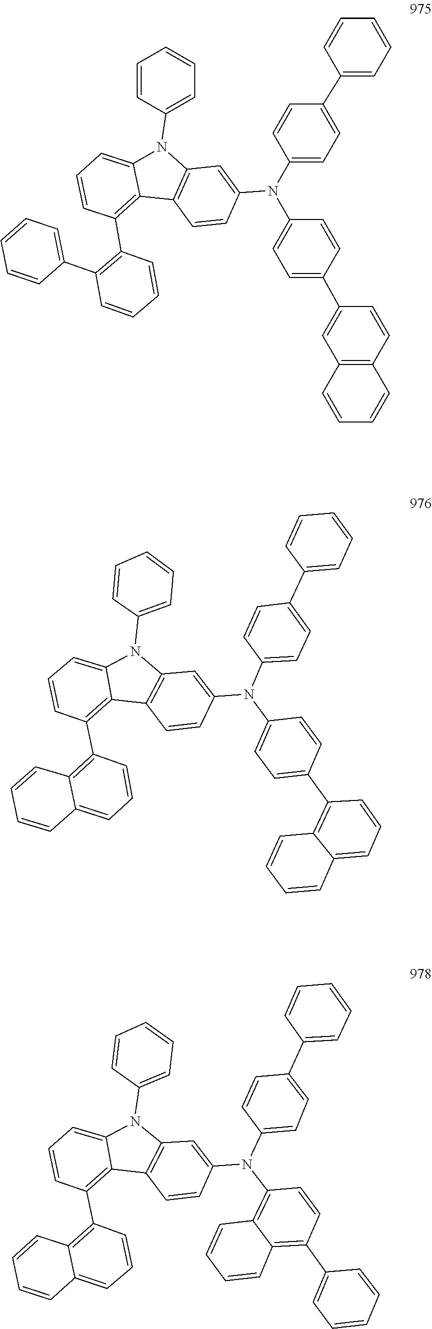 [SMILES: c1ccc(-c2ccc(N(c3ccc(-c4ccc5ccccc5c4)cc3)c3ccc4c5c(-c6ccccc6-c6ccccc6)cccc5n(-c5ccccc5)c4c3)cc2)cc1 ; c1ccc(-c2ccc(N(c3ccc(-c4cccc5ccccc45)cc3)c3ccc4c5c(-c6cccc7ccccc67)cccc5n(-c5ccccc5)c4c3)cc2)cc1 ; c1ccc(-c2ccc(N(c3ccc4c5c(-c6cccc7ccccc67)cccc5n(-c5ccccc5)c4c3)c3ccc(-c4ccccc4)c4ccccc34)cc2)cc1]

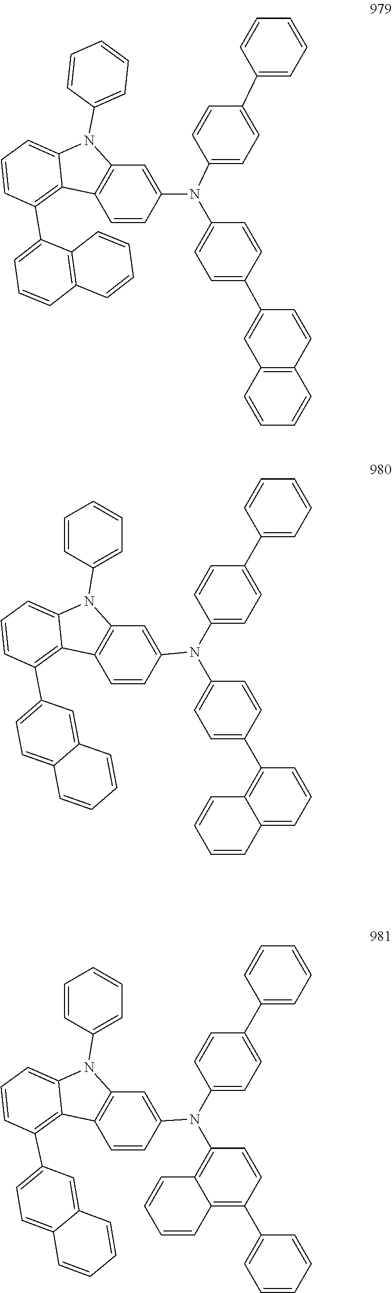 [SMILES: c1ccc(-c2ccc(N(c3ccc(-c4ccc5ccccc5c4)cc3)c3ccc4c5c(-c6cccc7ccccc67)cccc5n(-c5ccccc5)c4c3)cc2)cc1 ; c1ccc(-c2ccc(N(c3ccc(-c4cccc5ccccc45)cc3)c3ccc4c5c(-c6ccc7ccccc7c6)cccc5n(-c5ccccc5)c4c3)cc2)cc1 ; c1ccc(-c2ccc(N(c3ccc4c5c(-c6ccc7ccccc7c6)cccc5n(-c5ccccc5)c4c3)c3ccc(-c4ccccc4)c4ccccc34)cc2)cc1]

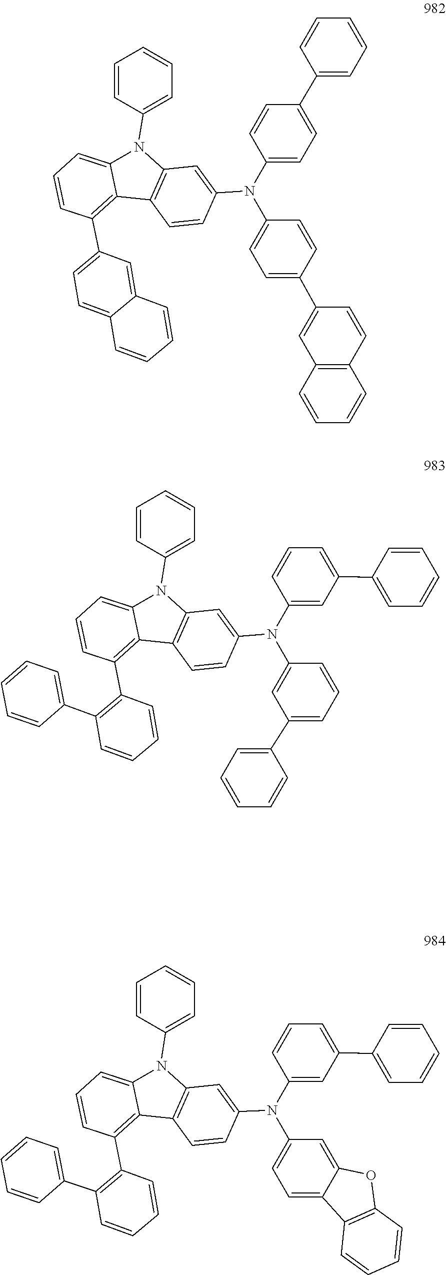 [SMILES: c1ccc(-c2ccc(N(c3ccc(-c4ccc5ccccc5c4)cc3)c3ccc4c5c(-c6ccc7ccccc7c6)cccc5n(-c5ccccc5)c4c3)cc2)cc1 ; c1ccc(-c2cccc(N(c3cccc(-c4ccccc4)c3)c3ccc4c5c(-c6ccccc6-c6ccccc6)cccc5n(-c5ccccc5)c4c3)c2)cc1 ; c1ccc(-c2cccc(N(c3ccc4c(c3)oc3ccccc34)c3ccc4c5c(-c6ccccc6-c6ccccc6)cccc5n(-c5ccccc5)c4c3)c2)cc1]

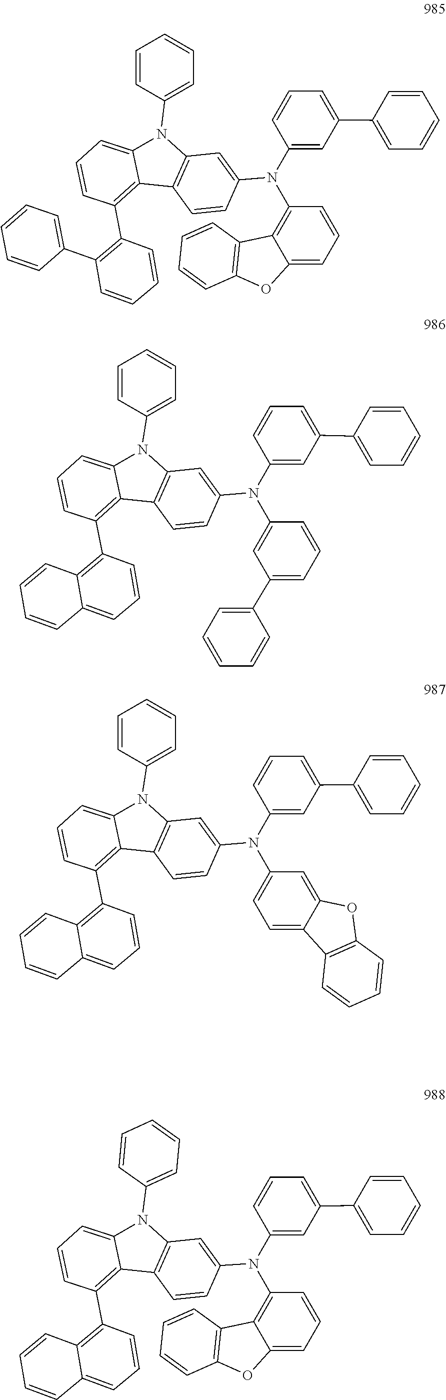 [SMILES: c1ccc(-c2cccc(N(c3ccc4c5c(-c6ccccc6-c6ccccc6)cccc5n(-c5ccccc5)c4c3)c3cccc4oc5ccccc5c34)c2)cc1 ; c1ccc(-c2cccc(N(c3cccc(-c4ccccc4)c3)c3ccc4c5c(-c6cccc7ccccc67)cccc5n(-c5ccccc5)c4c3)c2)cc1 ; c1ccc(-c2cccc(N(c3ccc4c(c3)oc3ccccc34)c3ccc4c5c(-c6cccc7ccccc67)cccc5n(-c5ccccc5)c4c3)c2)cc1 ; c1ccc(-c2cccc(N(c3ccc4c5c(-c6cccc7ccccc67)cccc5n(-c5ccccc5)c4c3)c3cccc4oc5ccccc5c34)c2)cc1]

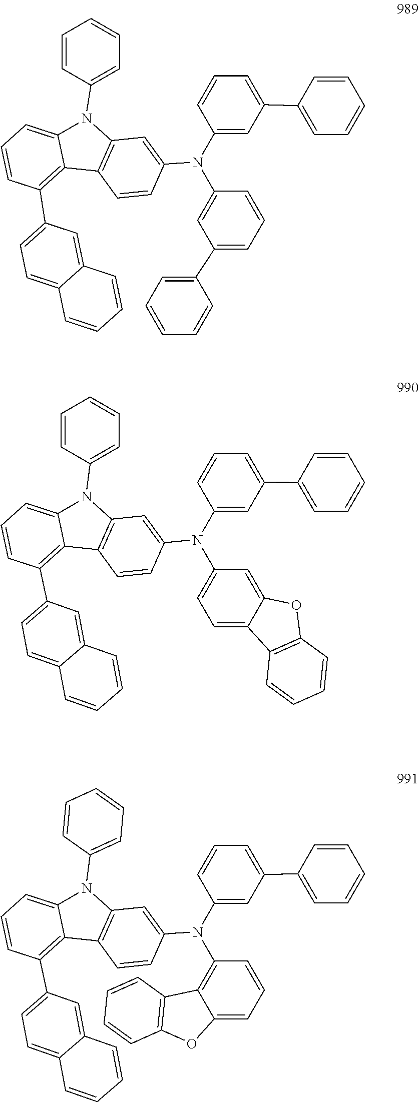 [SMILES: c1ccc(-c2cccc(N(c3cccc(-c4ccccc4)c3)c3ccc4c5c(-c6ccc7ccccc7c6)cccc5n(-c5ccccc5)c4c3)c2)cc1 ; c1ccc(-c2cccc(N(c3ccc4c(c3)oc3ccccc34)c3ccc4c5c(-c6ccc7ccccc7c6)cccc5n(-c5ccccc5)c4c3)c2)cc1 ; c1ccc(-c2cccc(N(c3ccc4c5c(-c6ccc7ccccc7c6)cccc5n(-c5ccccc5)c4c3)c3cccc4oc5ccccc5c34)c2)cc1]

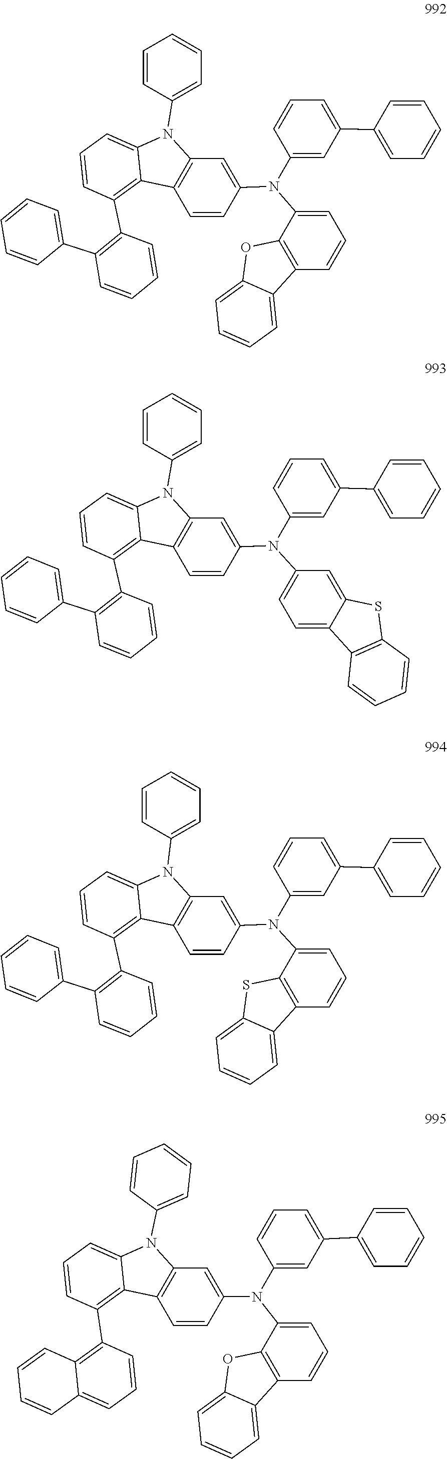 [SMILES: c1ccc(-c2cccc(N(c3ccc4c5c(-c6ccccc6-c6ccccc6)cccc5n(-c5ccccc5)c4c3)c3cccc4c3oc3ccccc34)c2)cc1 ; c1ccc(-c2cccc(N(c3ccc4c(c3)sc3ccccc34)c3ccc4c5c(-c6ccccc6-c6ccccc6)cccc5n(-c5ccccc5)c4c3)c2)cc1 ; c1ccc(-c2cccc(N(c3ccc4c5c(-c6ccccc6-c6ccccc6)cccc5n(-c5ccccc5)c4c3)c3cccc4c3sc3ccccc34)c2)cc1 ; c1ccc(-c2cccc(N(c3ccc4c5c(-c6cccc7ccccc67)cccc5n(-c5ccccc5)c4c3)c3cccc4c3oc3ccccc34)c2)cc1]

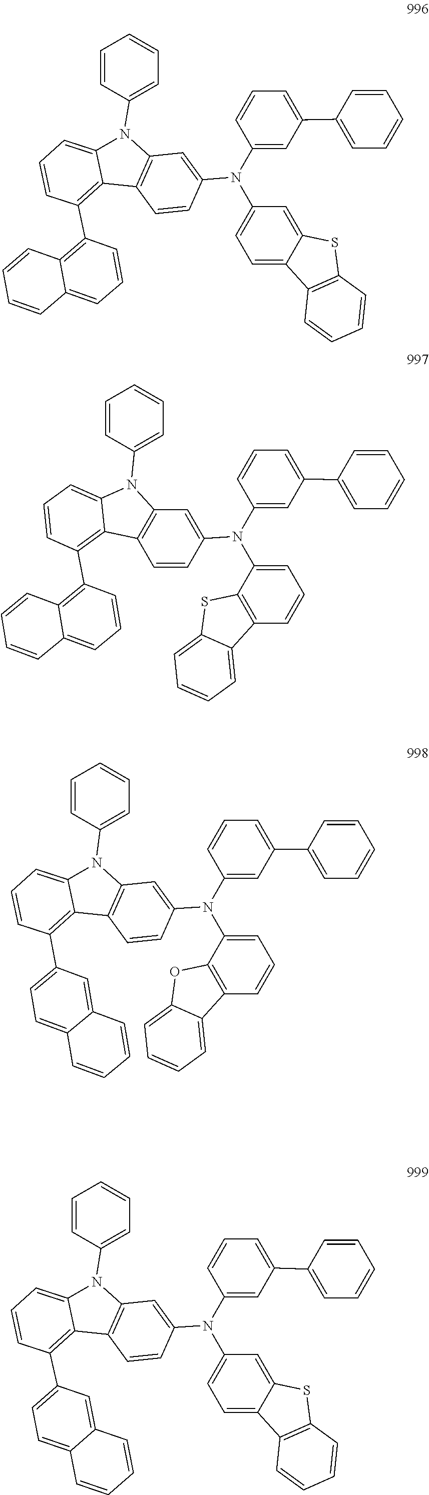 [SMILES: c1ccc(-c2cccc(N(c3ccc4c(c3)sc3ccccc34)c3ccc4c5c(-c6cccc7ccccc67)cccc5n(-c5ccccc5)c4c3)c2)cc1 ; c1ccc(-c2cccc(N(c3ccc4c5c(-c6cccc7ccccc67)cccc5n(-c5ccccc5)c4c3)c3cccc4c3sc3ccccc34)c2)cc1 ; c1ccc(-c2cccc(N(c3ccc4c5c(-c6ccc7ccccc7c6)cccc5n(-c5ccccc5)c4c3)c3cccc4c3oc3ccccc34)c2)cc1 ; c1ccc(-c2cccc(N(c3ccc4c(c3)sc3ccccc34)c3ccc4c5c(-c6ccc7ccccc7c6)cccc5n(-c5ccccc5)c4c3)c2)cc1]

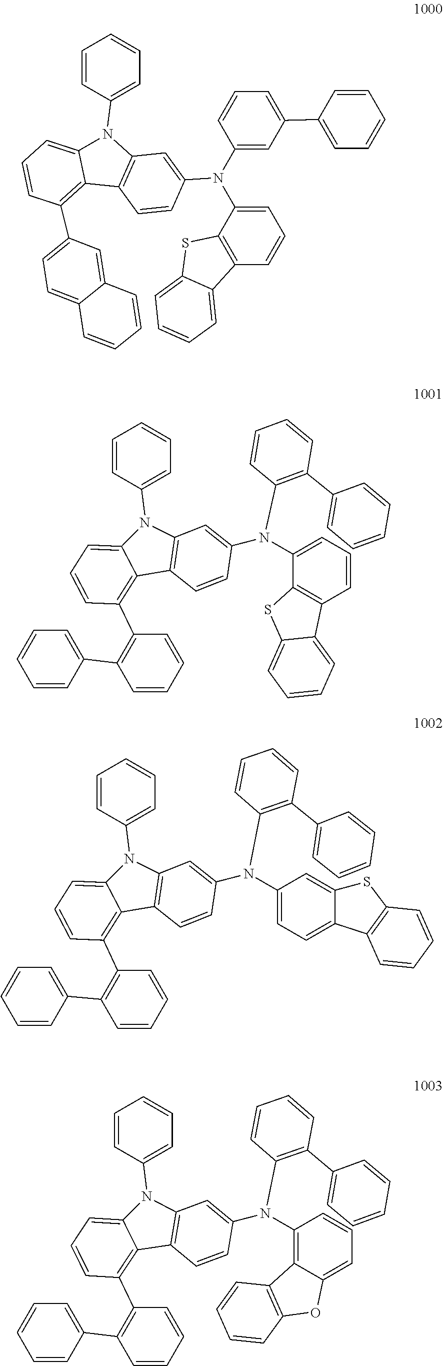 [SMILES: c1ccc(-c2cccc(N(c3ccc4c5c(-c6ccc7ccccc7c6)cccc5n(-c5ccccc5)c4c3)c3cccc4c3sc3ccccc34)c2)cc1 ; c1ccc(-c2ccccc2-c2cccc3c2c2ccc(N(c4ccccc4-c4ccccc4)c4cccc5c4sc4ccccc45)cc2n3-c2ccccc2)cc1 ; c1ccc(-c2ccccc2-c2cccc3c2c2ccc(N(c4ccc5c(c4)sc4ccccc45)c4ccccc4-c4ccccc4)cc2n3-c2ccccc2)cc1 ; c1ccc(-c2ccccc2-c2cccc3c2c2ccc(N(c4ccccc4-c4ccccc4)c4cccc5oc6ccccc6c45)cc2n3-c2ccccc2)cc1]

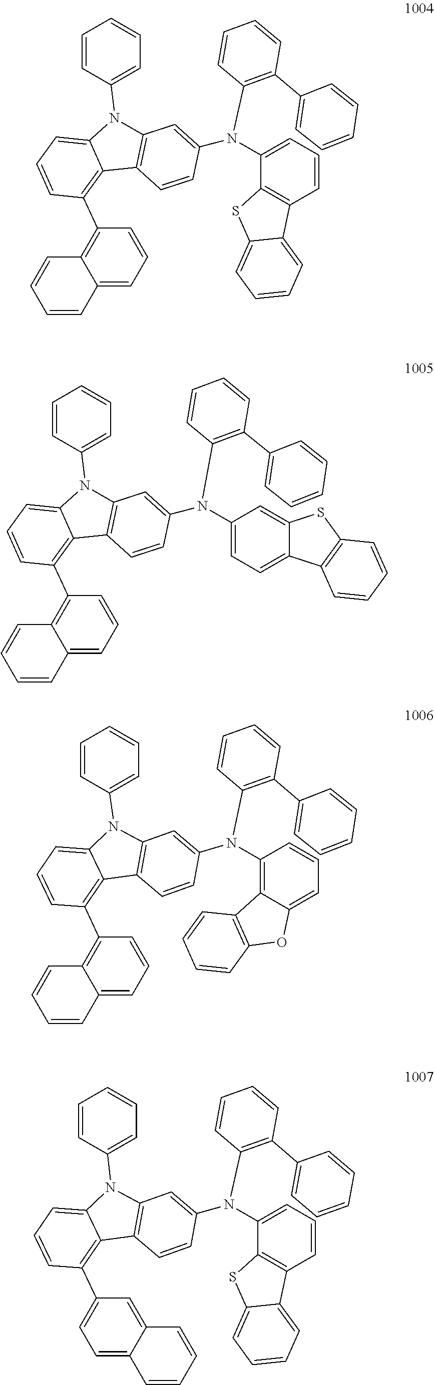 [SMILES: c1ccc(-c2ccccc2N(c2ccc3c4c(-c5cccc6ccccc56)cccc4n(-c4ccccc4)c3c2)c2cccc3c2sc2ccccc23)cc1 ; c1ccc(-c2ccccc2N(c2ccc3c(c2)sc2ccccc23)c2ccc3c4c(-c5cccc6ccccc56)cccc4n(-c4ccccc4)c3c2)cc1 ; c1ccc(-c2ccccc2N(c2ccc3c4c(-c5cccc6ccccc56)cccc4n(-c4ccccc4)c3c2)c2cccc3oc4ccccc4c23)cc1 ; c1ccc(-c2ccccc2N(c2ccc3c4c(-c5ccc6ccccc6c5)cccc4n(-c4ccccc4)c3c2)c2cccc3c2sc2ccccc23)cc1]

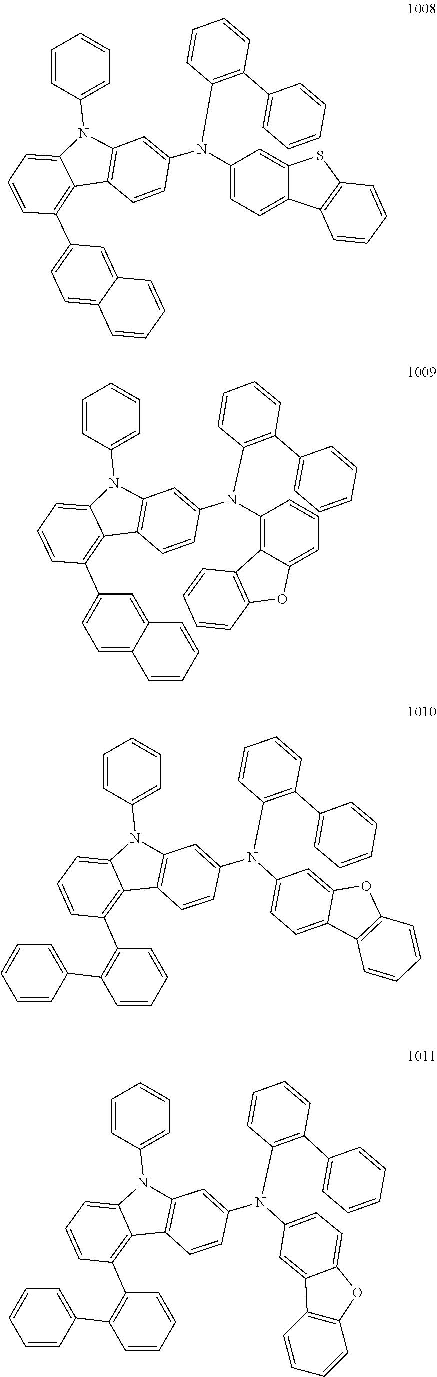 [SMILES: c1ccc(-c2ccccc2N(c2ccc3c(c2)sc2ccccc23)c2ccc3c4c(-c5ccc6ccccc6c5)cccc4n(-c4ccccc4)c3c2)cc1 ; c1ccc(-c2ccccc2N(c2ccc3c4c(-c5ccc6ccccc6c5)cccc4n(-c4ccccc4)c3c2)c2cccc3oc4ccccc4c23)cc1 ; c1ccc(-c2ccccc2-c2cccc3c2c2ccc(N(c4ccc5c(c4)oc4ccccc45)c4ccccc4-c4ccccc4)cc2n3-c2ccccc2)cc1 ; c1ccc(-c2ccccc2-c2cccc3c2c2ccc(N(c4ccc5oc6ccccc6c5c4)c4ccccc4-c4ccccc4)cc2n3-c2ccccc2)cc1]

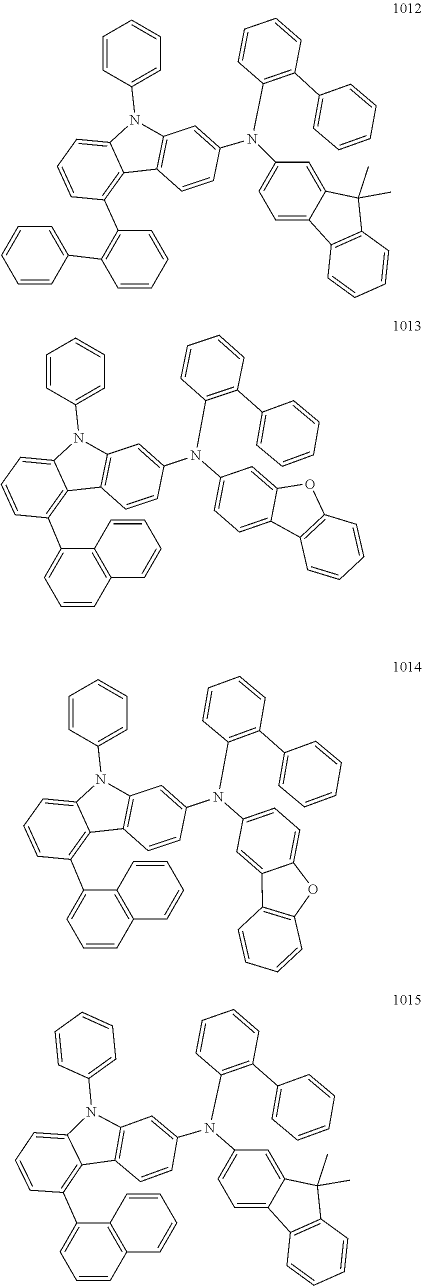 [SMILES: CC1(C)c2ccccc2-c2ccc(N(c3ccc4c5c(-c6ccccc6-c6ccccc6)cccc5n(-c5ccccc5)c4c3)c3ccccc3-c3ccccc3)cc21 ; c1ccc(-c2ccccc2N(c2ccc3c(c2)oc2ccccc23)c2ccc3c4c(-c5cccc6ccccc56)cccc4n(-c4ccccc4)c3c2)cc1 ; c1ccc(-c2ccccc2N(c2ccc3oc4ccccc4c3c2)c2ccc3c4c(-c5cccc6ccccc56)cccc4n(-c4ccccc4)c3c2)cc1 ; CC1(C)c2ccccc2-c2ccc(N(c3ccc4c5c(-c6cccc7ccccc67)cccc5n(-c5ccccc5)c4c3)c3ccccc3-c3ccccc3)cc21]

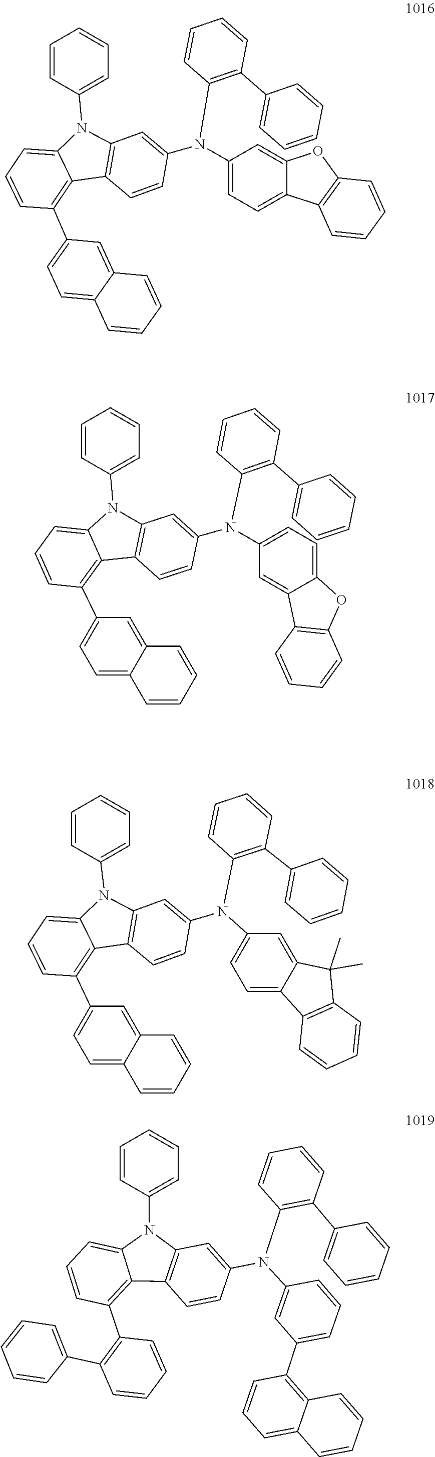

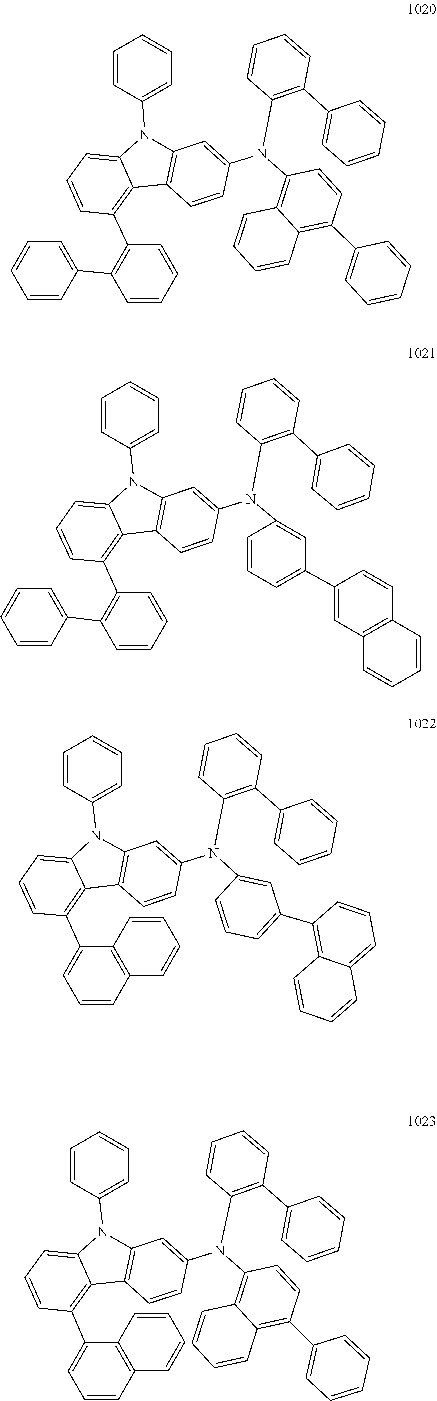 [SMILES: c1ccc(-c2ccccc2-c2cccc3c2c2ccc(N(c4ccccc4-c4ccccc4)c4ccc(-c5ccccc5)c5ccccc45)cc2n3-c2ccccc2)cc1 ; c1ccc(-c2ccccc2-c2cccc3c2c2ccc(N(c4cccc(-c5ccc6ccccc6c5)c4)c4ccccc4-c4ccccc4)cc2n3-c2ccccc2)cc1 ; c1ccc(-c2ccccc2N(c2cccc(-c3cccc4ccccc34)c2)c2ccc3c4c(-c5cccc6ccccc56)cccc4n(-c4ccccc4)c3c2)cc1 ; c1ccc(-c2ccccc2N(c2ccc3c4c(-c5cccc6ccccc56)cccc4n(-c4ccccc4)c3c2)c2ccc(-c3ccccc3)c3ccccc23)cc1]

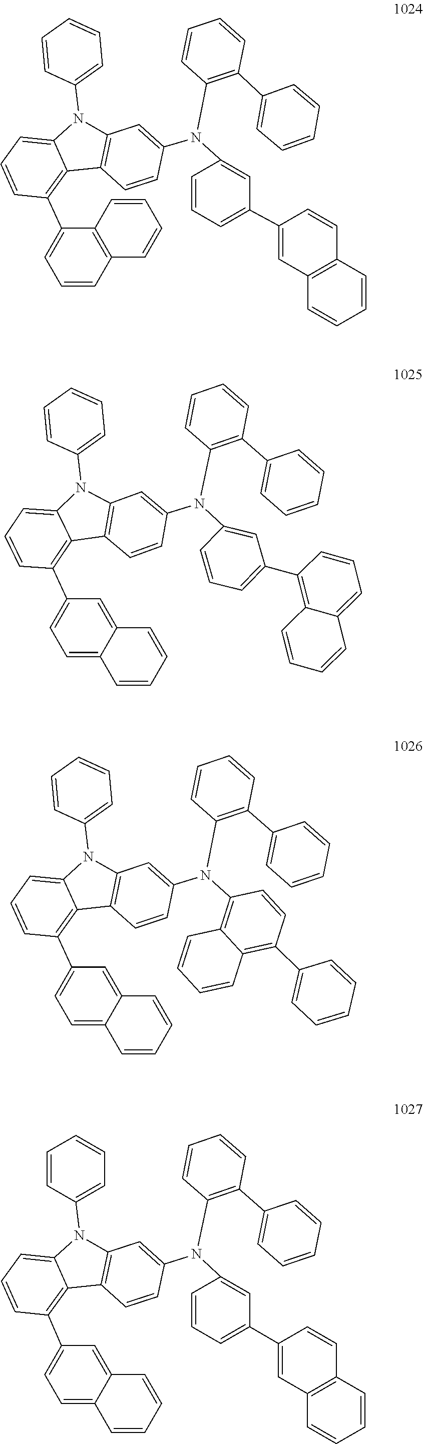 [SMILES: c1ccc(-c2ccccc2N(c2cccc(-c3ccc4ccccc4c3)c2)c2ccc3c4c(-c5cccc6ccccc56)cccc4n(-c4ccccc4)c3c2)cc1 ; c1ccc(-c2ccccc2N(c2cccc(-c3cccc4ccccc34)c2)c2ccc3c4c(-c5ccc6ccccc6c5)cccc4n(-c4ccccc4)c3c2)cc1 ; c1ccc(-c2ccccc2N(c2ccc3c4c(-c5ccc6ccccc6c5)cccc4n(-c4ccccc4)c3c2)c2ccc(-c3ccccc3)c3ccccc23)cc1 ; c1ccc(-c2ccccc2N(c2cccc(-c3ccc4ccccc4c3)c2)c2ccc3c4c(-c5ccc6ccccc6c5)cccc4n(-c4ccccc4)c3c2)cc1]

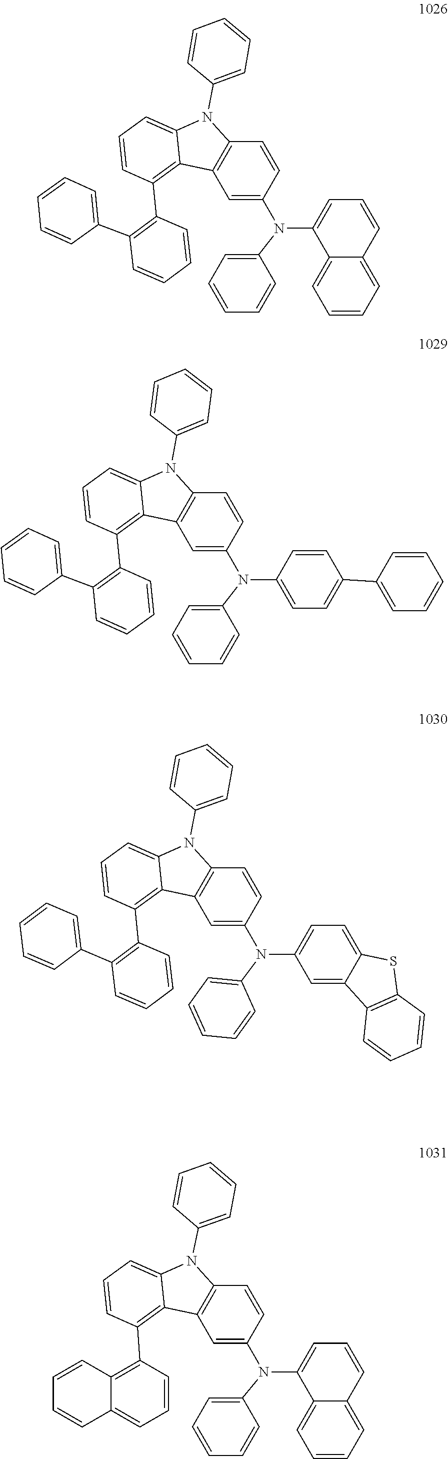 [SMILES: c1ccc(-c2ccccc2-c2cccc3c2c2cc(N(c4ccccc4)c4cccc5ccccc45)ccc2n3-c2ccccc2)cc1 ; c1ccc(-c2ccc(N(c3ccccc3)c3ccc4c(c3)c3c(-c5ccccc5-c5ccccc5)cccc3n4-c3ccccc3)cc2)cc1 ; c1ccc(-c2ccccc2-c2cccc3c2c2cc(N(c4ccccc4)c4ccc5sc6ccccc6c5c4)ccc2n3-c2ccccc2)cc1 ; c1ccc(N(c2ccc3c(c2)c2c(-c4cccc5ccccc45)cccc2n3-c2ccccc2)c2cccc3ccccc23)cc1]

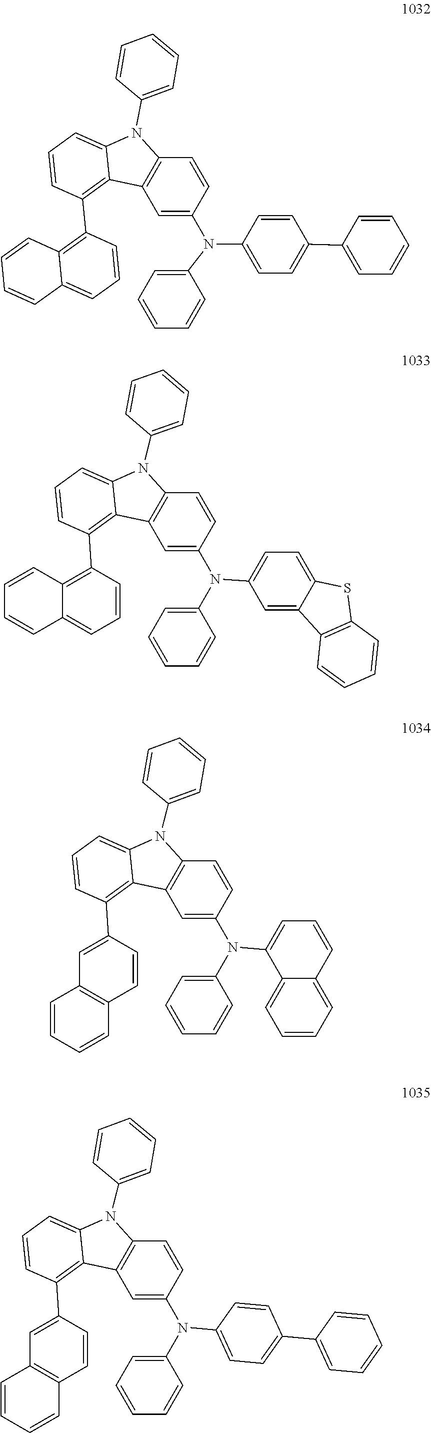

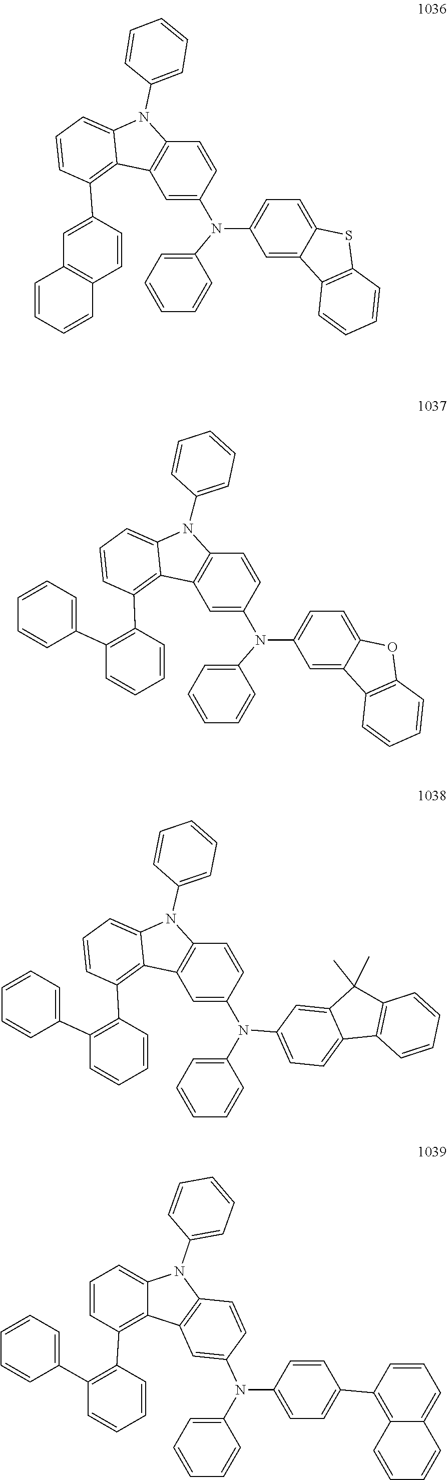 [SMILES: c1ccc(N(c2ccc3sc4ccccc4c3c2)c2ccc3c(c2)c2c(-c4ccc5ccccc5c4)cccc2n3-c2ccccc2)cc1 ; c1ccc(-c2ccccc2-c2cccc3c2c2cc(N(c4ccccc4)c4ccc5oc6ccccc6c5c4)ccc2n3-c2ccccc2)cc1 ; CC1(C)c2ccccc2-c2ccc(N(c3ccccc3)c3ccc4c(c3)c3c(-c5ccccc5-c5ccccc5)cccc3n4-c3ccccc3)cc21 ; c1ccc(-c2ccccc2-c2cccc3c2c2cc(N(c4ccccc4)c4ccc(-c5cccc6ccccc56)cc4)ccc2n3-c2ccccc2)cc1]

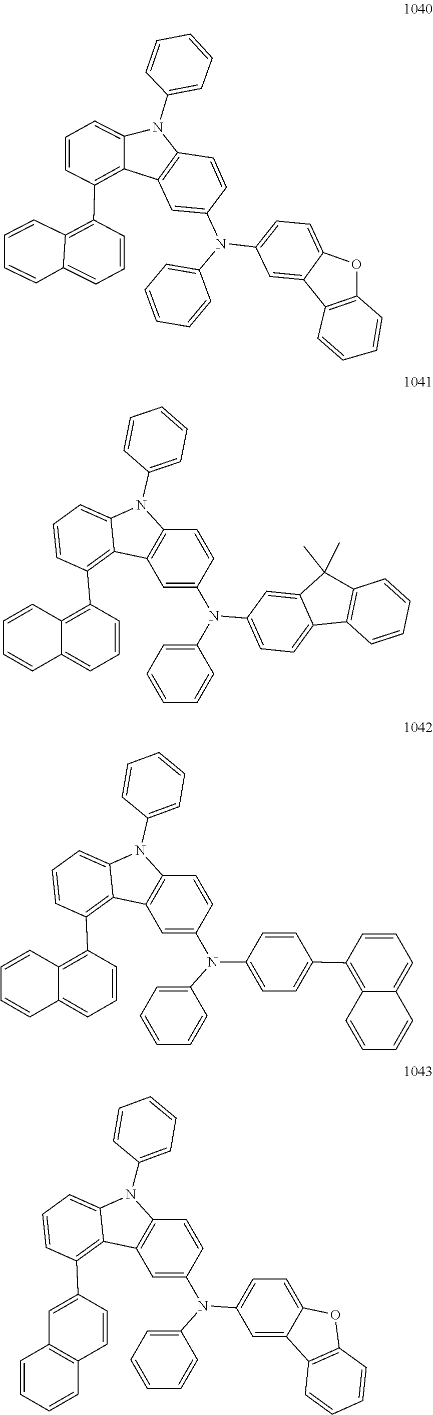 [SMILES: c1ccc(N(c2ccc3oc4ccccc4c3c2)c2ccc3c(c2)c2c(-c4cccc5ccccc45)cccc2n3-c2ccccc2)cc1 ; CC1(C)c2ccccc2-c2ccc(N(c3ccccc3)c3ccc4c(c3)c3c(-c5cccc6ccccc56)cccc3n4-c3ccccc3)cc21 ; c1ccc(N(c2ccc(-c3cccc4ccccc34)cc2)c2ccc3c(c2)c2c(-c4cccc5ccccc45)cccc2n3-c2ccccc2)cc1 ; c1ccc(N(c2ccc3oc4ccccc4c3c2)c2ccc3c(c2)c2c(-c4ccc5ccccc5c4)cccc2n3-c2ccccc2)cc1]

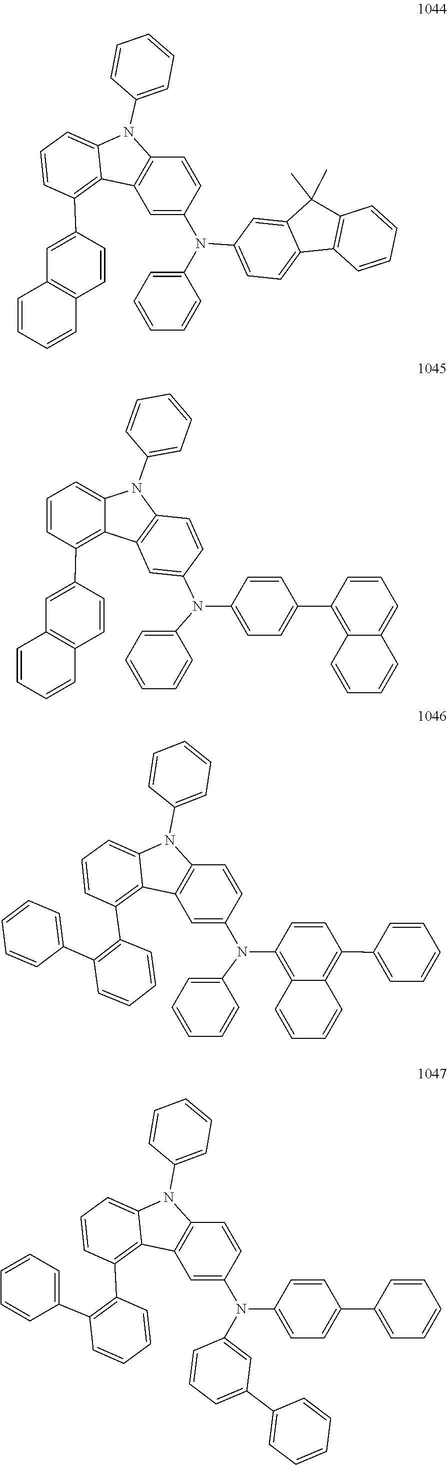 [SMILES: CC1(C)c2ccccc2-c2ccc(N(c3ccccc3)c3ccc4c(c3)c3c(-c5ccc6ccccc6c5)cccc3n4-c3ccccc3)cc21 ; c1ccc(N(c2ccc(-c3cccc4ccccc34)cc2)c2ccc3c(c2)c2c(-c4ccc5ccccc5c4)cccc2n3-c2ccccc2)cc1 ; c1ccc(-c2ccccc2-c2cccc3c2c2cc(N(c4ccccc4)c4ccc(-c5ccccc5)c5ccccc45)ccc2n3-c2ccccc2)cc1 ; c1ccc(-c2ccc(N(c3cccc(-c4ccccc4)c3)c3ccc4c(c3)c3c(-c5ccccc5-c5ccccc5)cccc3n4-c3ccccc3)cc2)cc1]

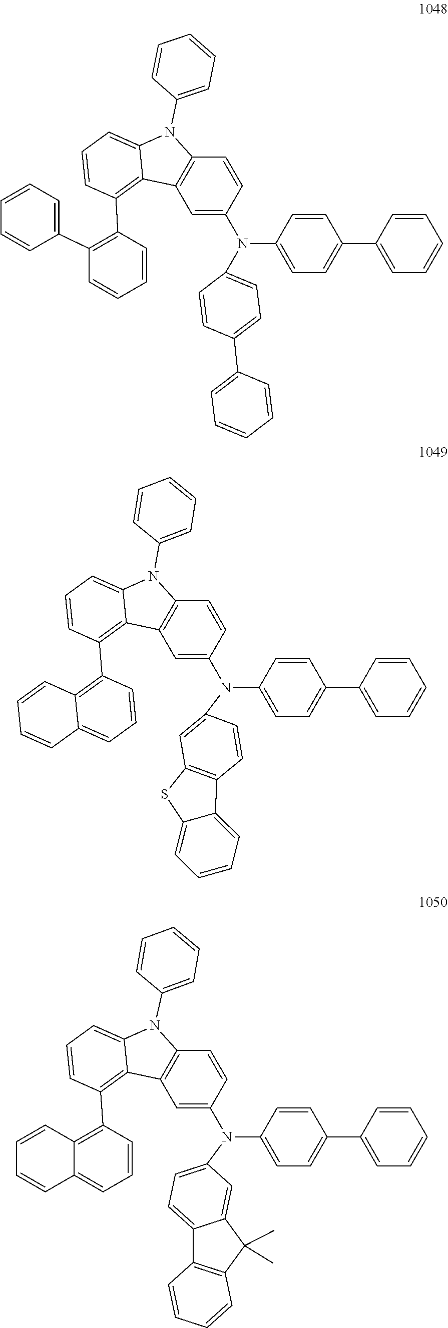 [SMILES: c1ccc(-c2ccc(N(c3ccc(-c4ccccc4)cc3)c3ccc4c(c3)c3c(-c5ccccc5-c5ccccc5)cccc3n4-c3ccccc3)cc2)cc1 ; c1ccc(-c2ccc(N(c3ccc4c(c3)sc3ccccc34)c3ccc4c(c3)c3c(-c5cccc6ccccc56)cccc3n4-c3ccccc3)cc2)cc1 ; CC1(C)c2ccccc2-c2ccc(N(c3ccc(-c4ccccc4)cc3)c3ccc4c(c3)c3c(-c5cccc6ccccc56)cccc3n4-c3ccccc3)cc21]

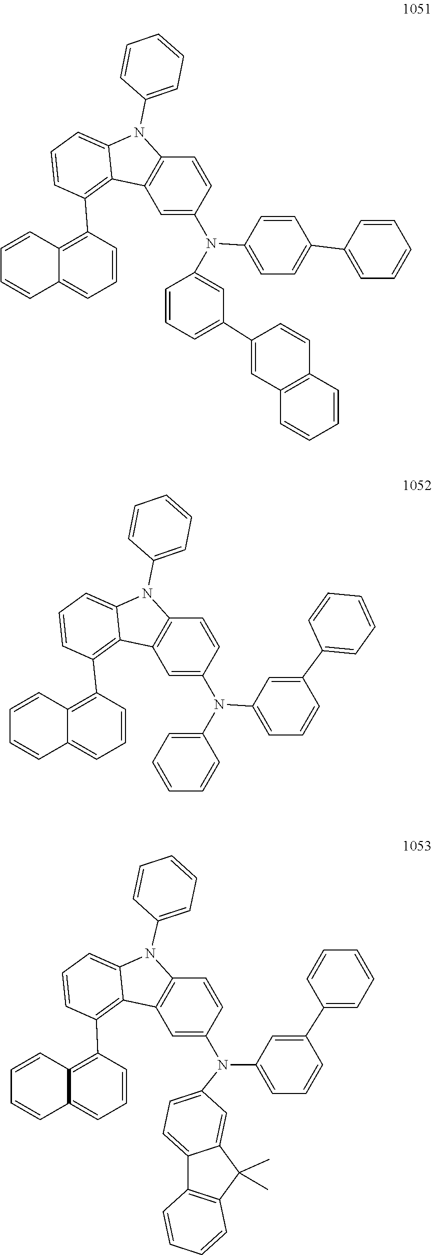

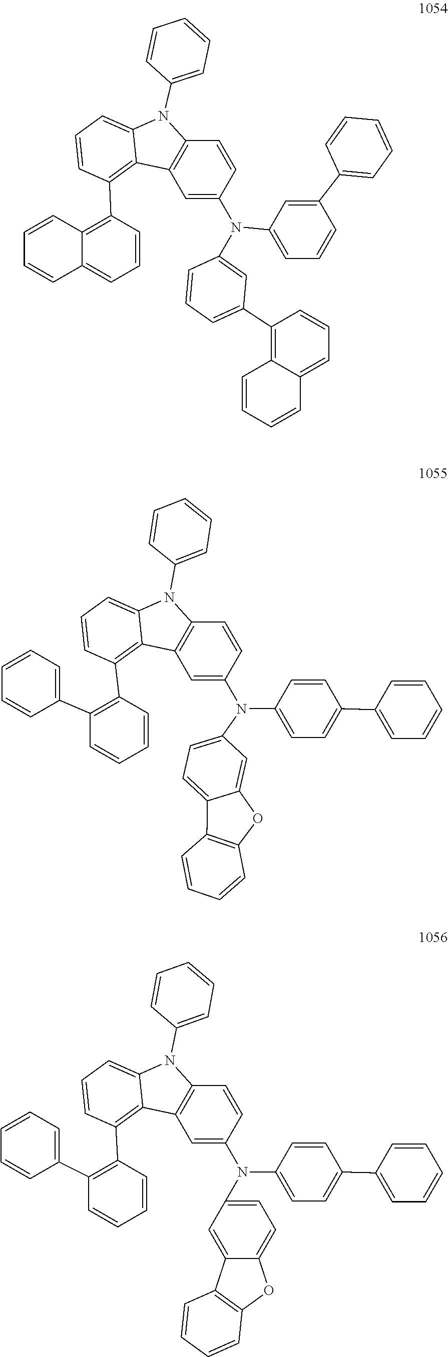 [SMILES: c1ccc(-c2cccc(N(c3cccc(-c4cccc5ccccc45)c3)c3ccc4c(c3)c3c(-c5cccc6ccccc56)cccc3n4-c3ccccc3)c2)cc1 ; c1ccc(-c2ccc(N(c3ccc4c(c3)oc3ccccc34)c3ccc4c(c3)c3c(-c5ccccc5-c5ccccc5)cccc3n4-c3ccccc3)cc2)cc1 ; c1ccc(-c2ccc(N(c3ccc4oc5ccccc5c4c3)c3ccc4c(c3)c3c(-c5ccccc5-c5ccccc5)cccc3n4-c3ccccc3)cc2)cc1]

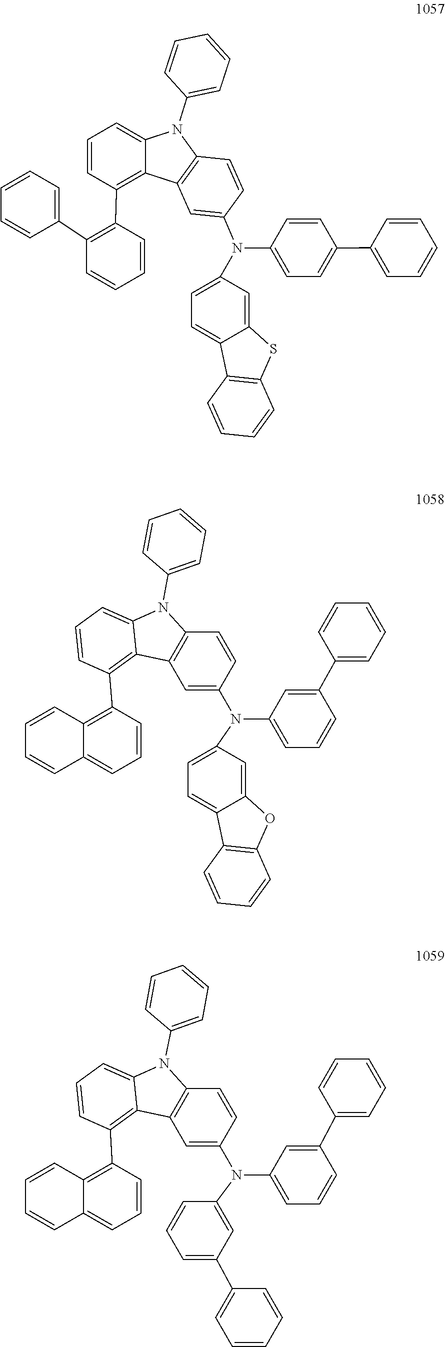 [SMILES: c1ccc(-c2ccc(N(c3ccc4c(c3)sc3ccccc34)c3ccc4c(c3)c3c(-c5ccccc5-c5ccccc5)cccc3n4-c3ccccc3)cc2)cc1 ; c1ccc(-c2cccc(N(c3ccc4c(c3)oc3ccccc34)c3ccc4c(c3)c3c(-c5cccc6ccccc56)cccc3n4-c3ccccc3)c2)cc1 ; c1ccc(-c2cccc(N(c3cccc(-c4ccccc4)c3)c3ccc4c(c3)c3c(-c5cccc6ccccc56)cccc3n4-c3ccccc3)c2)cc1]

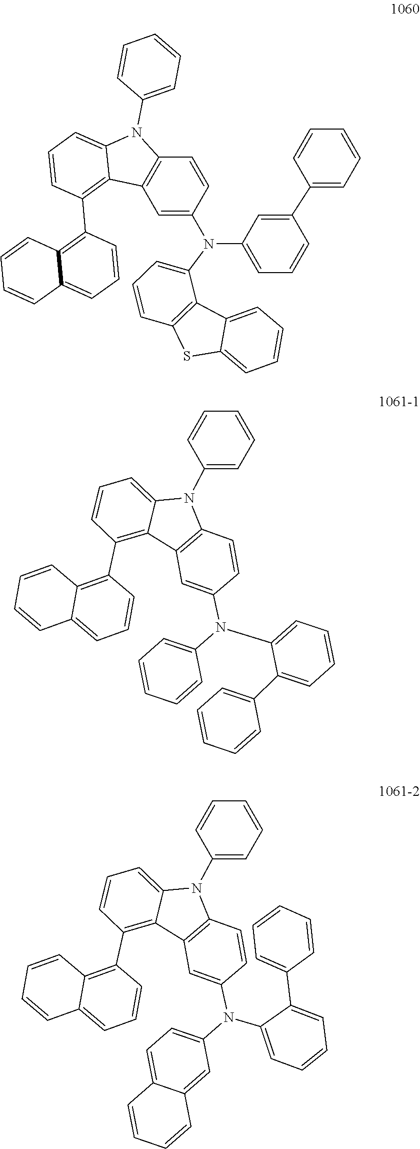

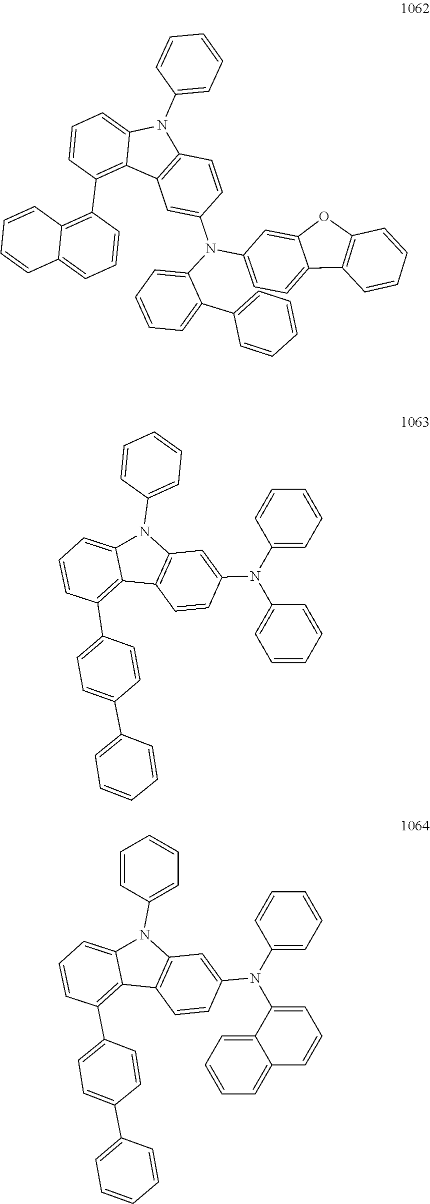

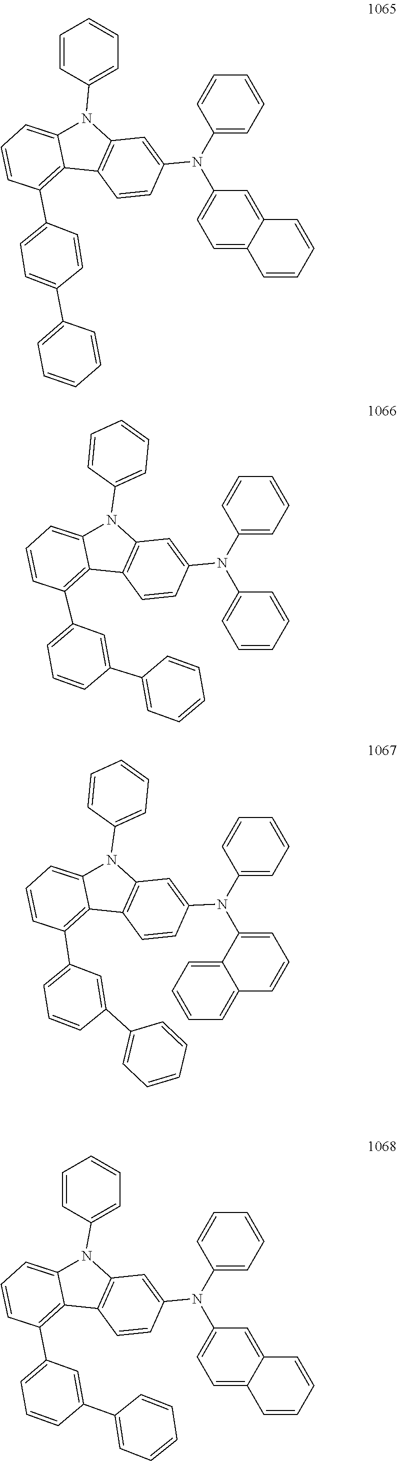 [SMILES: c1ccc(-c2ccc(-c3cccc4c3c3ccc(N(c5ccccc5)c5ccc6ccccc6c5)cc3n4-c3ccccc3)cc2)cc1 ; c1ccc(-c2cccc(-c3cccc4c3c3ccc(N(c5ccccc5)c5ccccc5)cc3n4-c3ccccc3)c2)cc1 ; c1ccc(-c2cccc(-c3cccc4c3c3ccc(N(c5ccccc5)c5cccc6ccccc56)cc3n4-c3ccccc3)c2)cc1 ; c1ccc(-c2cccc(-c3cccc4c3c3ccc(N(c5ccccc5)c5ccc6ccccc6c5)cc3n4-c3ccccc3)c2)cc1]

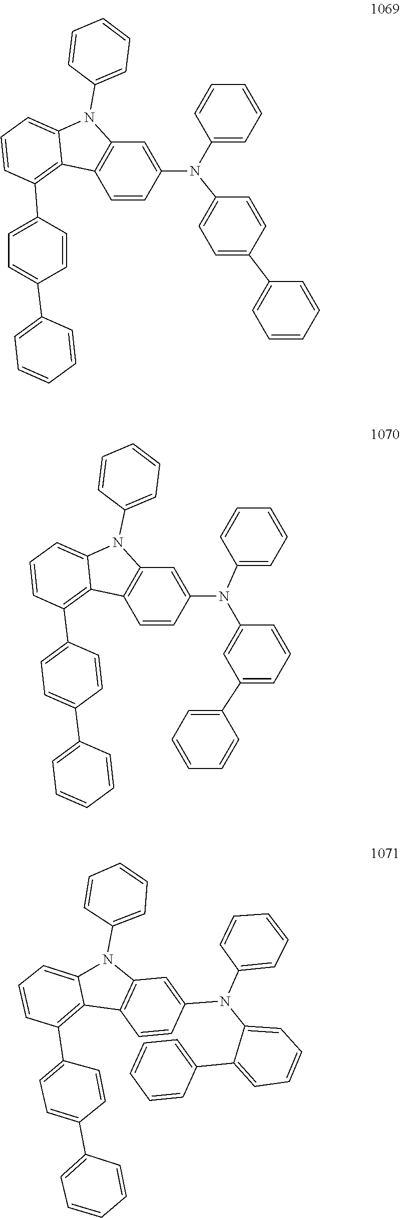 [SMILES: c1ccc(-c2ccc(-c3cccc4c3c3ccc(N(c5ccccc5)c5ccc(-c6ccccc6)cc5)cc3n4-c3ccccc3)cc2)cc1 ; c1ccc(-c2ccc(-c3cccc4c3c3ccc(N(c5ccccc5)c5cccc(-c6ccccc6)c5)cc3n4-c3ccccc3)cc2)cc1 ; c1ccc(-c2ccc(-c3cccc4c3c3ccc(N(c5ccccc5)c5ccccc5-c5ccccc5)cc3n4-c3ccccc3)cc2)cc1]

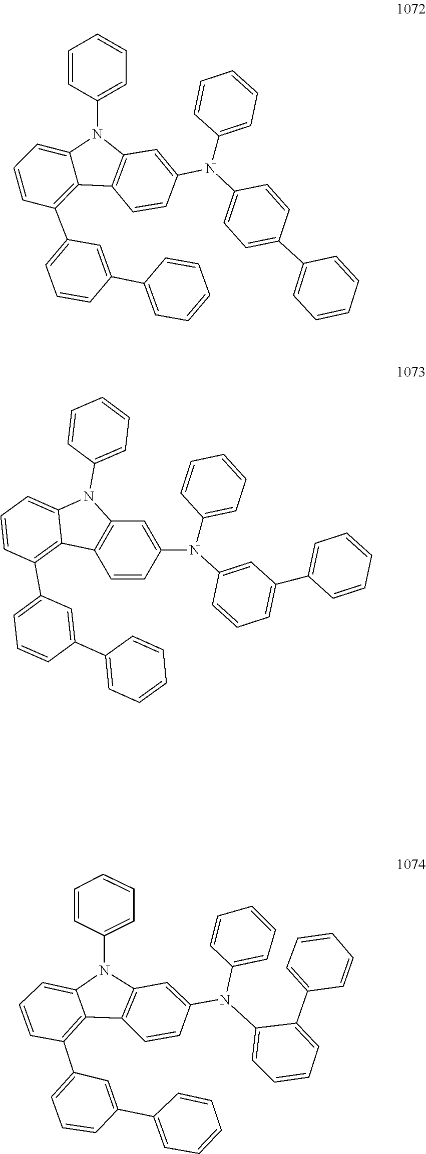 [SMILES: c1ccc(-c2ccc(N(c3ccccc3)c3ccc4c5c(-c6cccc(-c7ccccc7)c6)cccc5n(-c5ccccc5)c4c3)cc2)cc1 ; c1ccc(-c2cccc(-c3cccc4c3c3ccc(N(c5ccccc5)c5cccc(-c6ccccc6)c5)cc3n4-c3ccccc3)c2)cc1 ; c1ccc(-c2cccc(-c3cccc4c3c3ccc(N(c5ccccc5)c5ccccc5-c5ccccc5)cc3n4-c3ccccc3)c2)cc1]

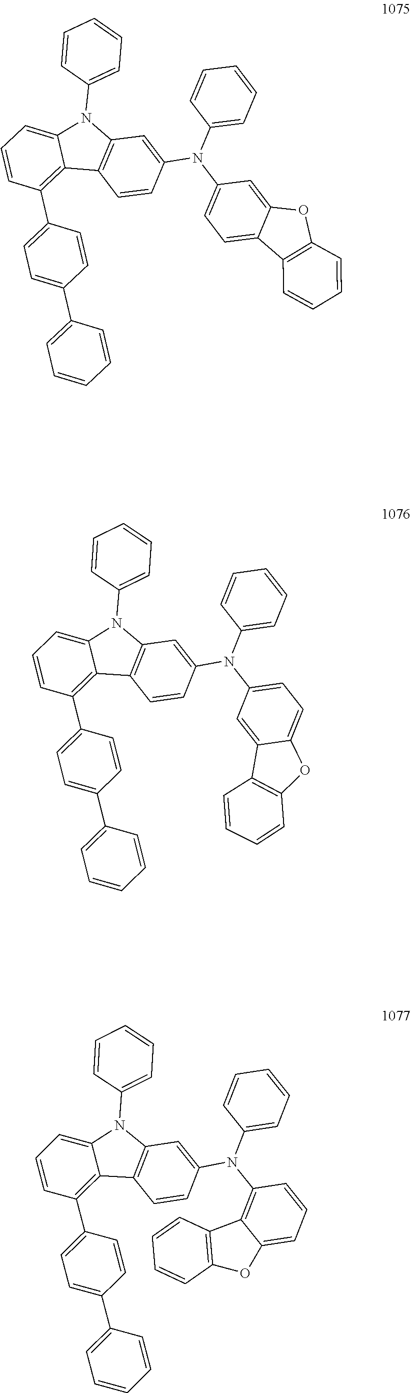 [SMILES: c1ccc(-c2ccc(-c3cccc4c3c3ccc(N(c5ccccc5)c5ccc6c(c5)oc5ccccc56)cc3n4-c3ccccc3)cc2)cc1 ; c1ccc(-c2ccc(-c3cccc4c3c3ccc(N(c5ccccc5)c5ccc6oc7ccccc7c6c5)cc3n4-c3ccccc3)cc2)cc1 ; c1ccc(-c2ccc(-c3cccc4c3c3ccc(N(c5ccccc5)c5cccc6oc7ccccc7c56)cc3n4-c3ccccc3)cc2)cc1]

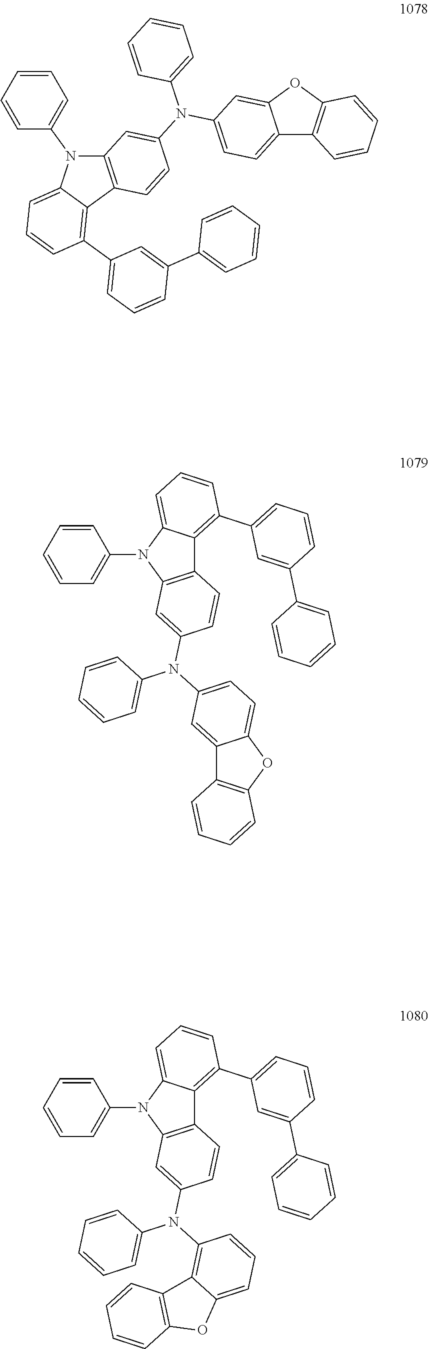 [SMILES: c1ccc(-c2cccc(-c3cccc4c3c3ccc(N(c5ccccc5)c5ccc6c(c5)oc5ccccc56)cc3n4-c3ccccc3)c2)cc1 ; c1ccc(-c2cccc(-c3cccc4c3c3ccc(N(c5ccccc5)c5ccc6oc7ccccc7c6c5)cc3n4-c3ccccc3)c2)cc1 ; c1ccc(-c2cccc(-c3cccc4c3c3ccc(N(c5ccccc5)c5cccc6oc7ccccc7c56)cc3n4-c3ccccc3)c2)cc1]

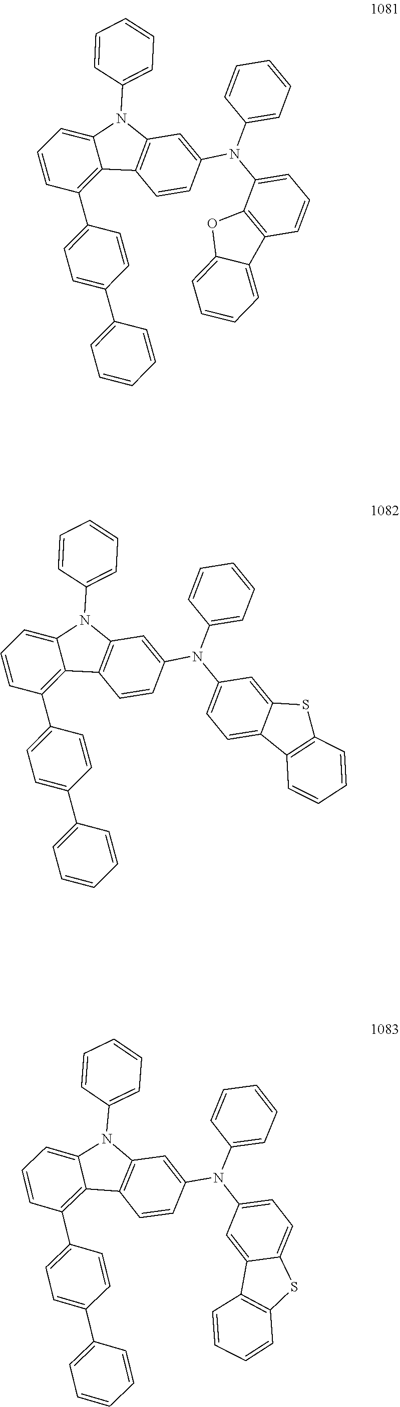 [SMILES: c1ccc(-c2ccc(-c3cccc4c3c3ccc(N(c5ccccc5)c5cccc6c5oc5ccccc56)cc3n4-c3ccccc3)cc2)cc1 ; c1ccc(-c2ccc(-c3cccc4c3c3ccc(N(c5ccccc5)c5ccc6c(c5)sc5ccccc56)cc3n4-c3ccccc3)cc2)cc1 ; c1ccc(-c2ccc(-c3cccc4c3c3ccc(N(c5ccccc5)c5ccc6sc7ccccc7c6c5)cc3n4-c3ccccc3)cc2)cc1]

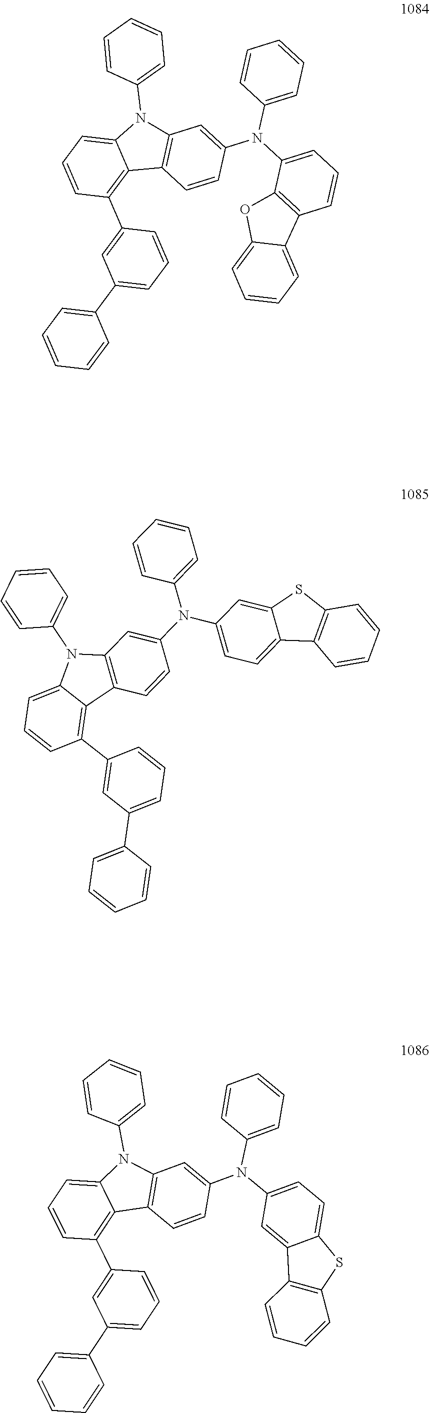 [SMILES: c1ccc(-c2cccc(-c3cccc4c3c3ccc(N(c5ccccc5)c5cccc6c5oc5ccccc56)cc3n4-c3ccccc3)c2)cc1 ; c1ccc(-c2cccc(-c3cccc4c3c3ccc(N(c5ccccc5)c5ccc6c(c5)sc5ccccc56)cc3n4-c3ccccc3)c2)cc1 ; c1ccc(-c2cccc(-c3cccc4c3c3ccc(N(c5ccccc5)c5ccc6sc7ccccc7c6c5)cc3n4-c3ccccc3)c2)cc1]

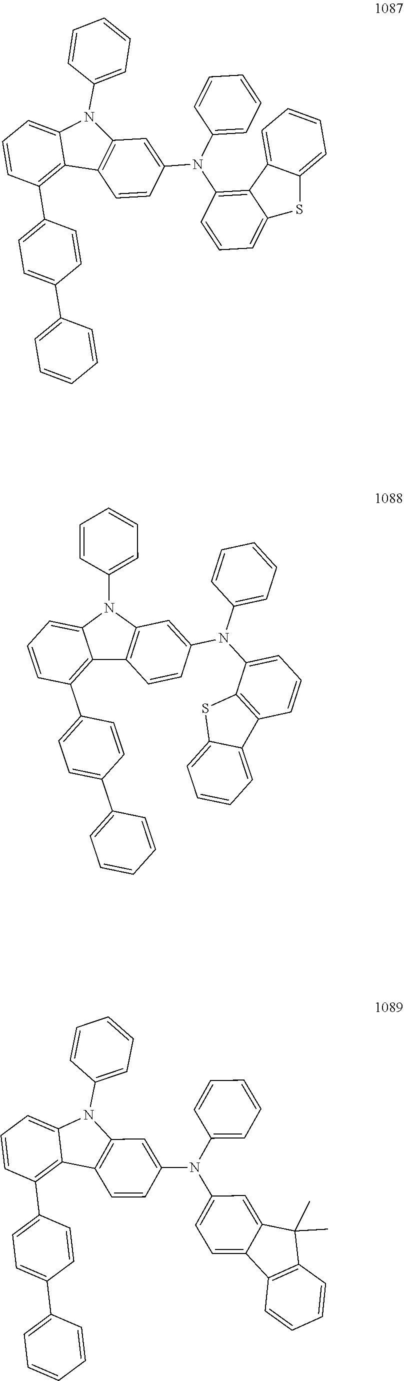 [SMILES: c1ccc(-c2ccc(-c3cccc4c3c3ccc(N(c5ccccc5)c5cccc6sc7ccccc7c56)cc3n4-c3ccccc3)cc2)cc1 ; c1ccc(-c2ccc(-c3cccc4c3c3ccc(N(c5ccccc5)c5cccc6c5sc5ccccc56)cc3n4-c3ccccc3)cc2)cc1 ; CC1(C)c2ccccc2-c2ccc(N(c3ccccc3)c3ccc4c5c(-c6ccc(-c7ccccc7)cc6)cccc5n(-c5ccccc5)c4c3)cc21]

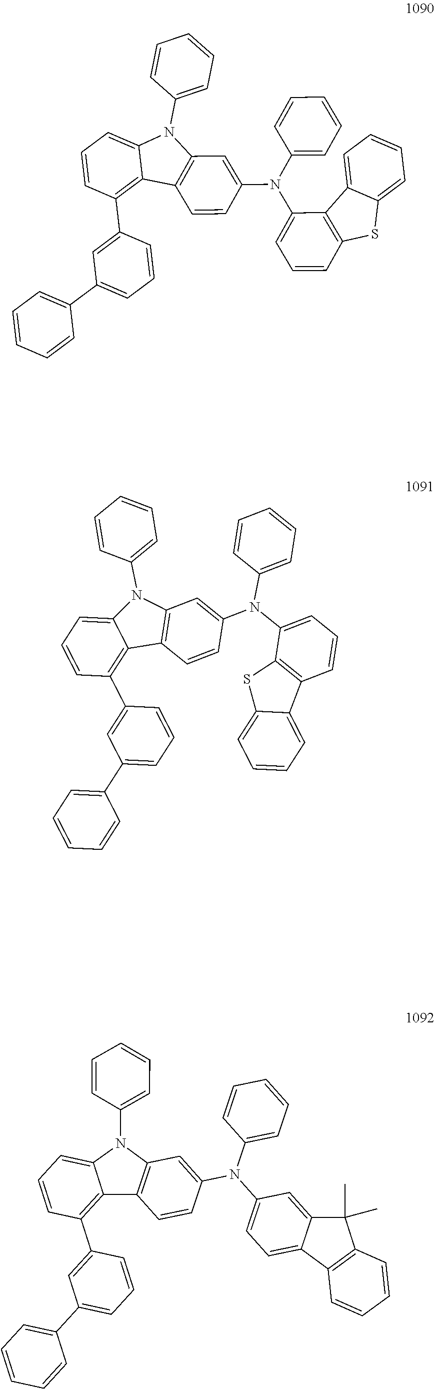 [SMILES: c1ccc(-c2cccc(-c3cccc4c3c3ccc(N(c5ccccc5)c5cccc6sc7ccccc7c56)cc3n4-c3ccccc3)c2)cc1 ; c1ccc(-c2cccc(-c3cccc4c3c3ccc(N(c5ccccc5)c5cccc6c5sc5ccccc56)cc3n4-c3ccccc3)c2)cc1 ; CC1(C)c2ccccc2-c2ccc(N(c3ccccc3)c3ccc4c5c(-c6cccc(-c7ccccc7)c6)cccc5n(-c5ccccc5)c4c3)cc21]

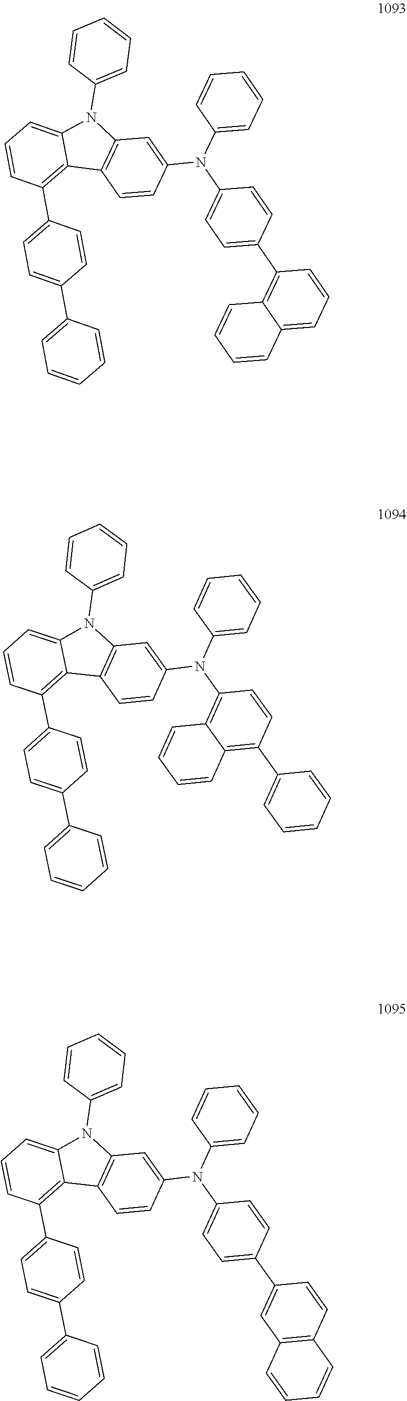 [SMILES: c1ccc(-c2ccc(-c3cccc4c3c3ccc(N(c5ccccc5)c5ccc(-c6cccc7ccccc67)cc5)cc3n4-c3ccccc3)cc2)cc1 ; c1ccc(-c2ccc(-c3cccc4c3c3ccc(N(c5ccccc5)c5ccc(-c6ccccc6)c6ccccc56)cc3n4-c3ccccc3)cc2)cc1 ; c1ccc(-c2ccc(-c3cccc4c3c3ccc(N(c5ccccc5)c5ccc(-c6ccc7ccccc7c6)cc5)cc3n4-c3ccccc3)cc2)cc1]

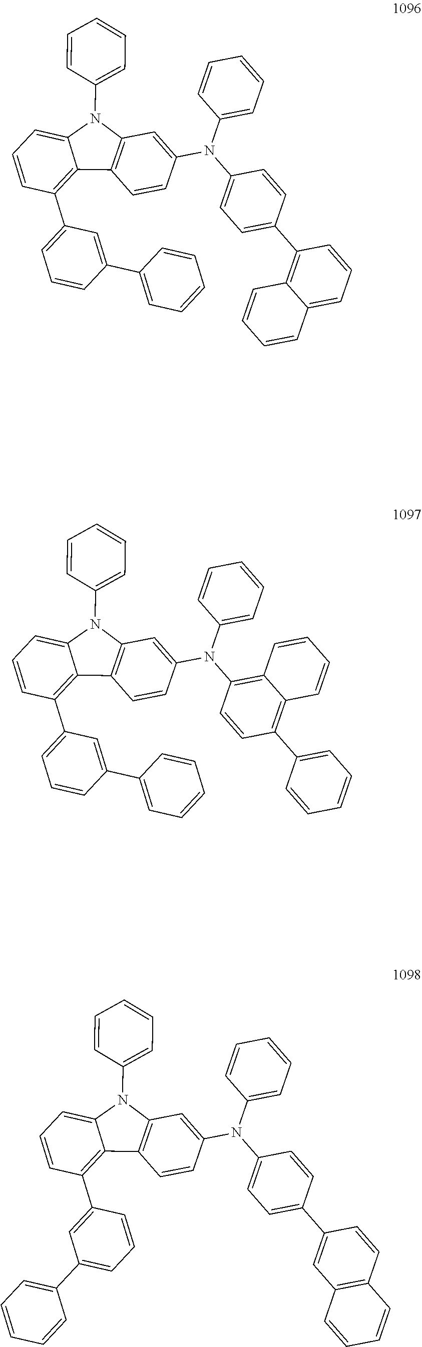 [SMILES: c1ccc(-c2cccc(-c3cccc4c3c3ccc(N(c5ccccc5)c5ccc(-c6cccc7ccccc67)cc5)cc3n4-c3ccccc3)c2)cc1 ; c1ccc(-c2cccc(-c3cccc4c3c3ccc(N(c5ccccc5)c5ccc(-c6ccccc6)c6ccccc56)cc3n4-c3ccccc3)c2)cc1 ; c1ccc(-c2cccc(-c3cccc4c3c3ccc(N(c5ccccc5)c5ccc(-c6ccc7ccccc7c6)cc5)cc3n4-c3ccccc3)c2)cc1]

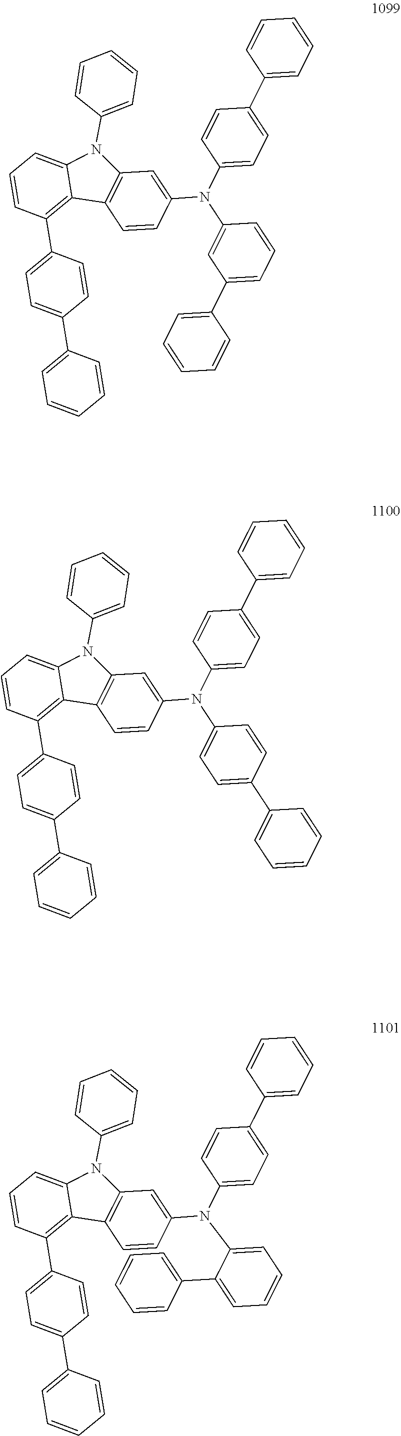 [SMILES: c1ccc(-c2ccc(-c3cccc4c3c3ccc(N(c5ccc(-c6ccccc6)cc5)c5cccc(-c6ccccc6)c5)cc3n4-c3ccccc3)cc2)cc1 ; c1ccc(-c2ccc(-c3cccc4c3c3ccc(N(c5ccc(-c6ccccc6)cc5)c5ccc(-c6ccccc6)cc5)cc3n4-c3ccccc3)cc2)cc1 ; c1ccc(-c2ccc(-c3cccc4c3c3ccc(N(c5ccc(-c6ccccc6)cc5)c5ccccc5-c5ccccc5)cc3n4-c3ccccc3)cc2)cc1]

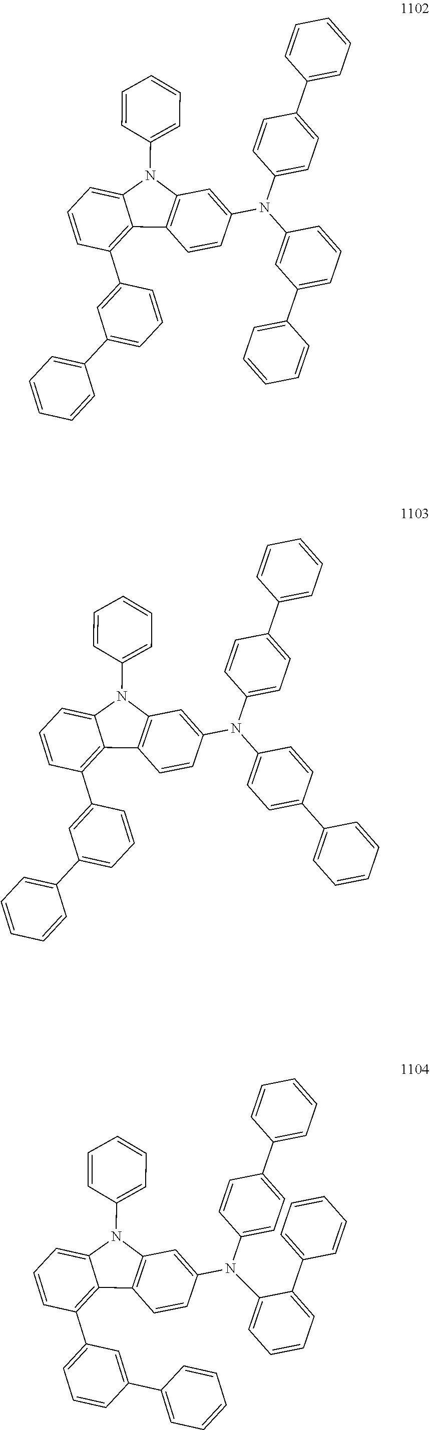 [SMILES: c1ccc(-c2ccc(N(c3cccc(-c4ccccc4)c3)c3ccc4c5c(-c6cccc(-c7ccccc7)c6)cccc5n(-c5ccccc5)c4c3)cc2)cc1 ; c1ccc(-c2ccc(N(c3ccc(-c4ccccc4)cc3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3ccccc3-c3ccccc3)cc2)cc1]

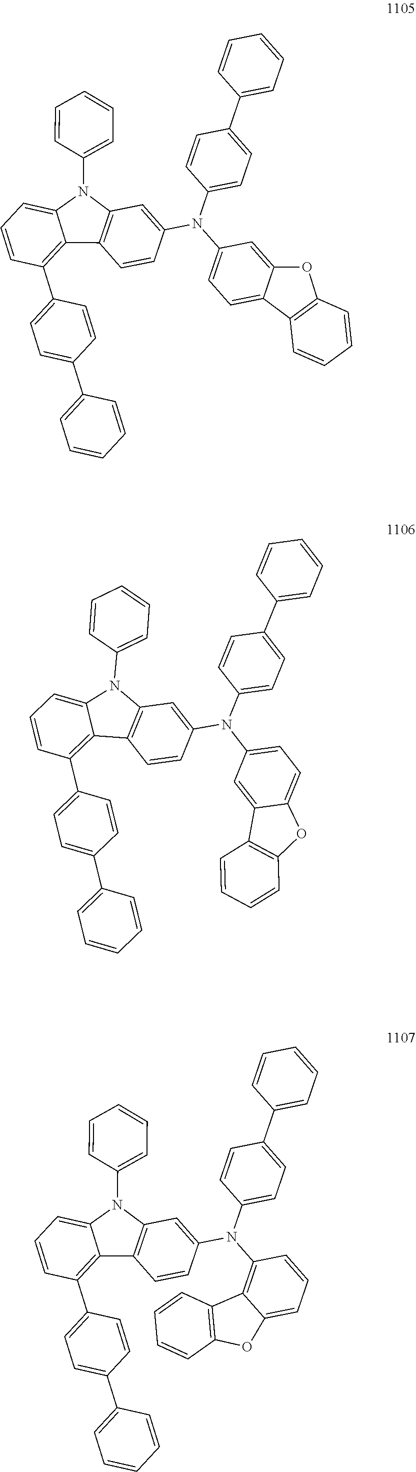 [SMILES: c1ccc(-c2ccc(-c3cccc4c3c3ccc(N(c5ccc(-c6ccccc6)cc5)c5ccc6c(c5)oc5ccccc56)cc3n4-c3ccccc3)cc2)cc1 ; c1ccc(-c2ccc(-c3cccc4c3c3ccc(N(c5ccc(-c6ccccc6)cc5)c5ccc6oc7ccccc7c6c5)cc3n4-c3ccccc3)cc2)cc1 ; c1ccc(-c2ccc(-c3cccc4c3c3ccc(N(c5ccc(-c6ccccc6)cc5)c5cccc6oc7ccccc7c56)cc3n4-c3ccccc3)cc2)cc1]

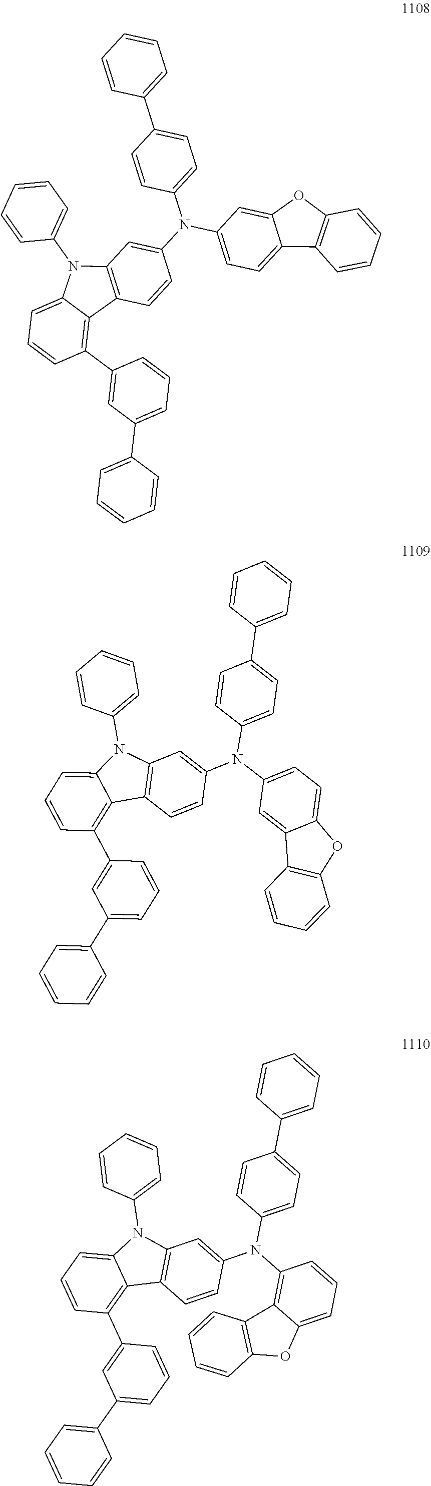 [SMILES: c1ccc(-c2ccc(N(c3ccc4c(c3)oc3ccccc34)c3ccc4c5c(-c6cccc(-c7ccccc7)c6)cccc5n(-c5ccccc5)c4c3)cc2)cc1 ; c1ccc(-c2ccc(N(c3ccc4oc5ccccc5c4c3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3cccc4oc5ccccc5c34)cc2)cc1]

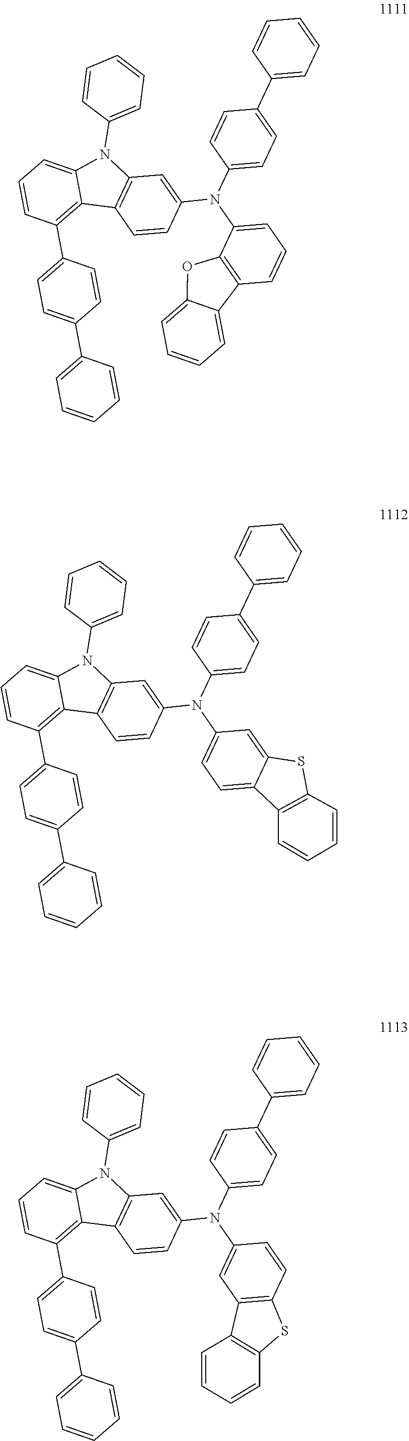 [SMILES: c1ccc(-c2ccc(-c3cccc4c3c3ccc(N(c5ccc(-c6ccccc6)cc5)c5cccc6c5oc5ccccc56)cc3n4-c3ccccc3)cc2)cc1 ; c1ccc(-c2ccc(-c3cccc4c3c3ccc(N(c5ccc(-c6ccccc6)cc5)c5ccc6c(c5)sc5ccccc56)cc3n4-c3ccccc3)cc2)cc1 ; c1ccc(-c2ccc(-c3cccc4c3c3ccc(N(c5ccc(-c6ccccc6)cc5)c5ccc6sc7ccccc7c6c5)cc3n4-c3ccccc3)cc2)cc1]

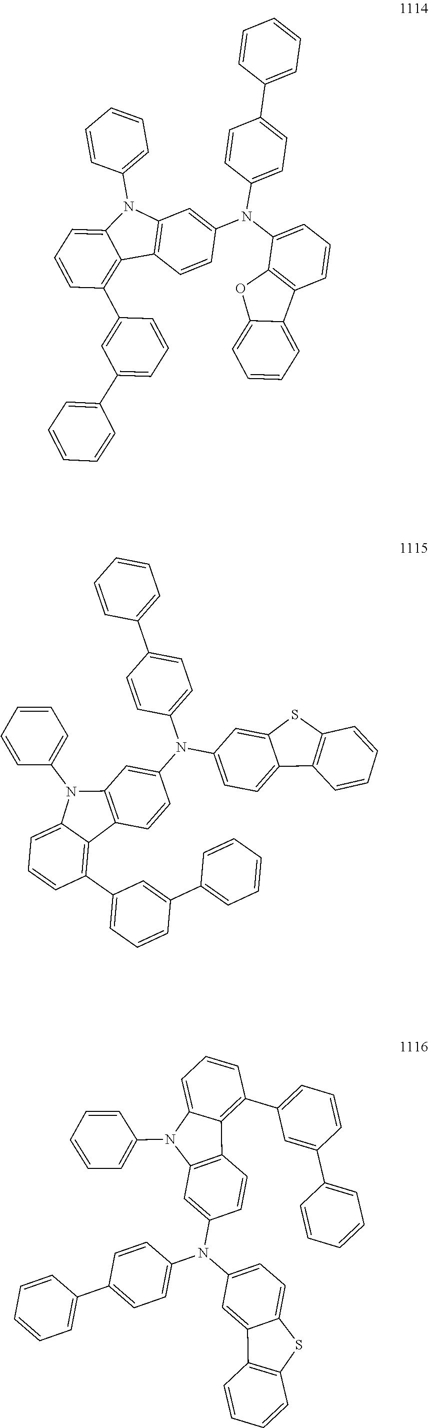 [SMILES: c1ccc(-c2ccc(N(c3ccc4c5c(-c6cccc(-c7ccccc7)c6)cccc5n(-c5ccccc5)c4c3)c3cccc4c3oc3ccccc34)cc2)cc1 ; c1ccc(-c2ccc(N(c3ccc4c(c3)sc3ccccc34)c3ccc4c5c(-c6cccc(-c7ccccc7)c6)cccc5n(-c5ccccc5)c4c3)cc2)cc1 ; c1ccc(-c2ccc(N(c3ccc4sc5ccccc5c4c3)c3ccc4c5c(-c6cccc(-c7ccccc7)c6)cccc5n(-c5ccccc5)c4c3)cc2)cc1]

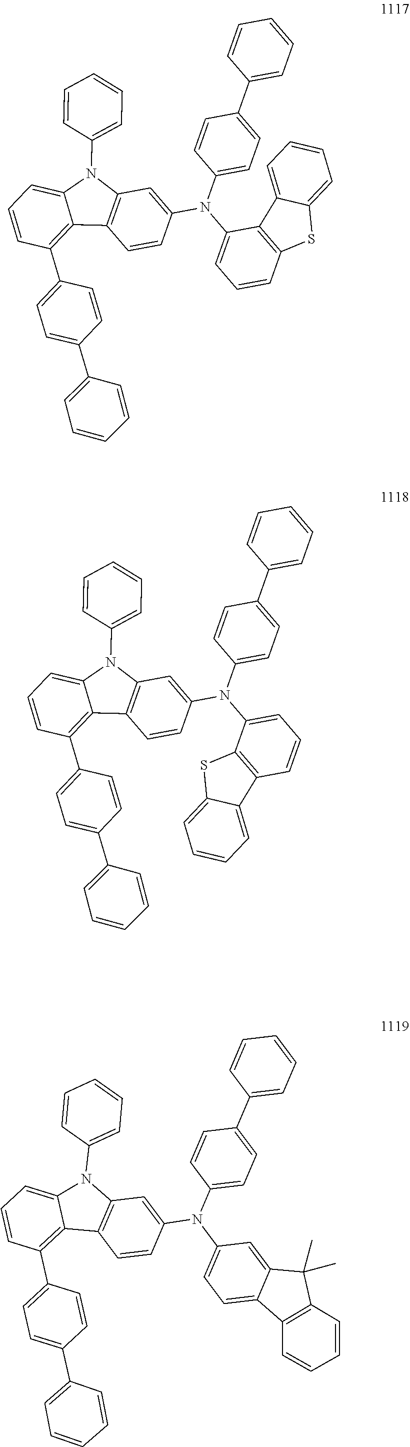 [SMILES: c1ccc(-c2ccc(-c3cccc4c3c3ccc(N(c5ccc(-c6ccccc6)cc5)c5cccc6sc7ccccc7c56)cc3n4-c3ccccc3)cc2)cc1 ; c1ccc(-c2ccc(-c3cccc4c3c3ccc(N(c5ccc(-c6ccccc6)cc5)c5cccc6c5sc5ccccc56)cc3n4-c3ccccc3)cc2)cc1 ; CC1(C)c2ccccc2-c2ccc(N(c3ccc(-c4ccccc4)cc3)c3ccc4c5c(-c6ccc(-c7ccccc7)cc6)cccc5n(-c5ccccc5)c4c3)cc21]

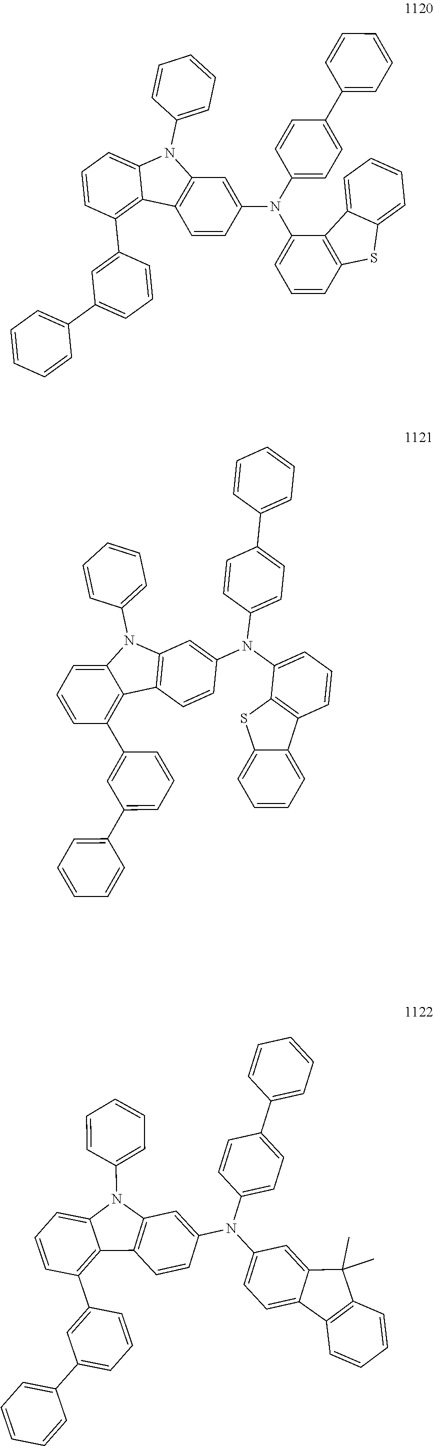 [SMILES: c1ccc(-c2ccc(N(c3ccc4c5c(-c6cccc(-c7ccccc7)c6)cccc5n(-c5ccccc5)c4c3)c3cccc4sc5ccccc5c34)cc2)cc1 ; c1ccc(-c2ccc(N(c3ccc4c5c(-c6cccc(-c7ccccc7)c6)cccc5n(-c5ccccc5)c4c3)c3cccc4c3sc3ccccc34)cc2)cc1 ; CC1(C)c2ccccc2-c2ccc(N(c3ccc(-c4ccccc4)cc3)c3ccc4c5c(-c6cccc(-c7ccccc7)c6)cccc5n(-c5ccccc5)c4c3)cc21]

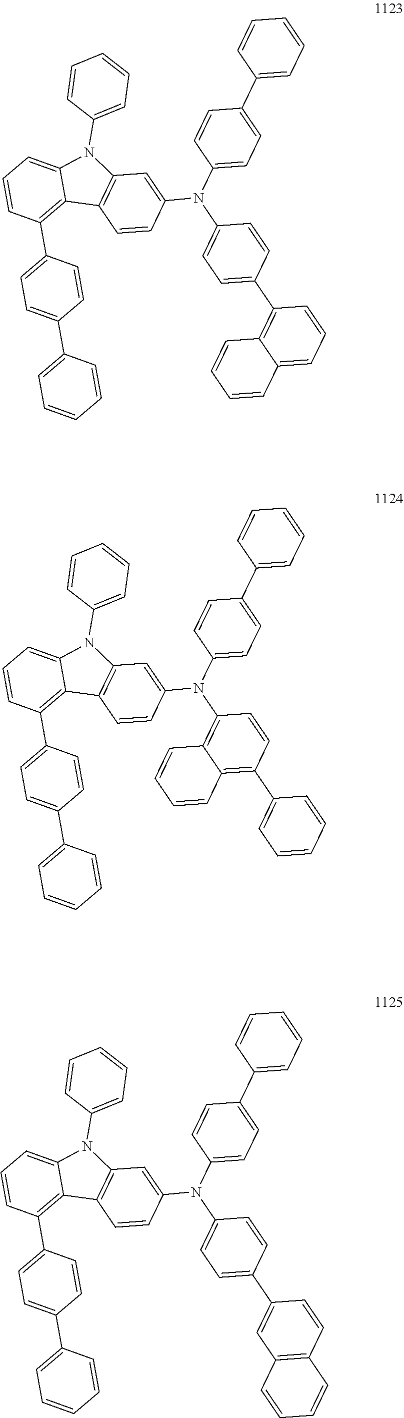 [SMILES: c1ccc(-c2ccc(-c3cccc4c3c3ccc(N(c5ccc(-c6ccccc6)cc5)c5ccc(-c6cccc7ccccc67)cc5)cc3n4-c3ccccc3)cc2)cc1 ; c1ccc(-c2ccc(-c3cccc4c3c3ccc(N(c5ccc(-c6ccccc6)cc5)c5ccc(-c6ccccc6)c6ccccc56)cc3n4-c3ccccc3)cc2)cc1 ; c1ccc(-c2ccc(-c3cccc4c3c3ccc(N(c5ccc(-c6ccccc6)cc5)c5ccc(-c6ccc7ccccc7c6)cc5)cc3n4-c3ccccc3)cc2)cc1]

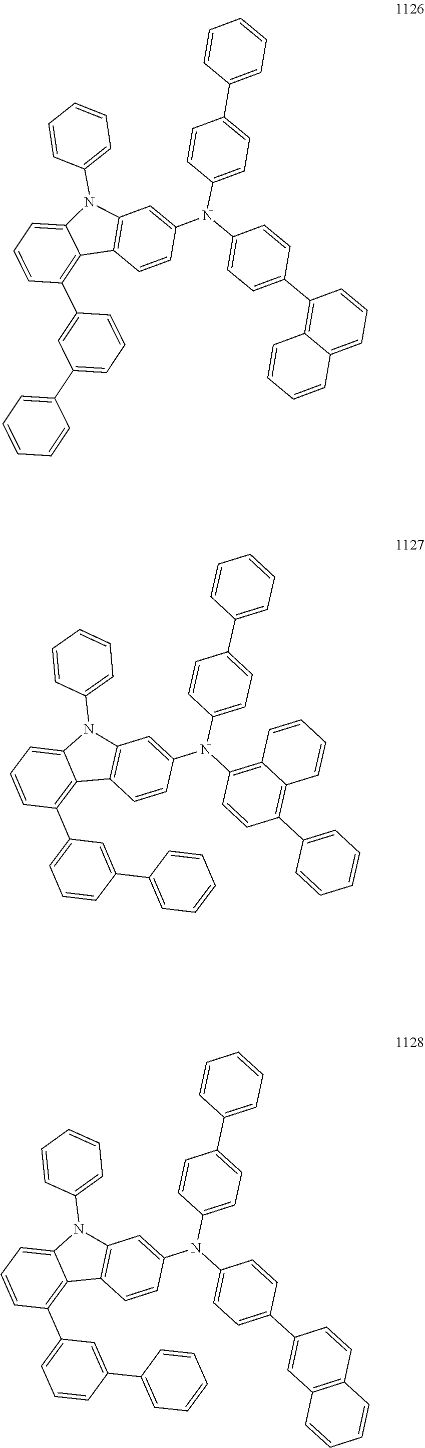 [SMILES: c1ccc(-c2ccc(N(c3ccc(-c4cccc5ccccc45)cc3)c3ccc4c5c(-c6cccc(-c7ccccc7)c6)cccc5n(-c5ccccc5)c4c3)cc2)cc1 ; c1ccc(-c2ccc(N(c3ccc4c5c(-c6cccc(-c7ccccc7)c6)cccc5n(-c5ccccc5)c4c3)c3ccc(-c4ccccc4)c4ccccc34)cc2)cc1 ; c1ccc(-c2ccc(N(c3ccc(-c4ccc5ccccc5c4)cc3)c3ccc4c5c(-c6cccc(-c7ccccc7)c6)cccc5n(-c5ccccc5)c4c3)cc2)cc1]

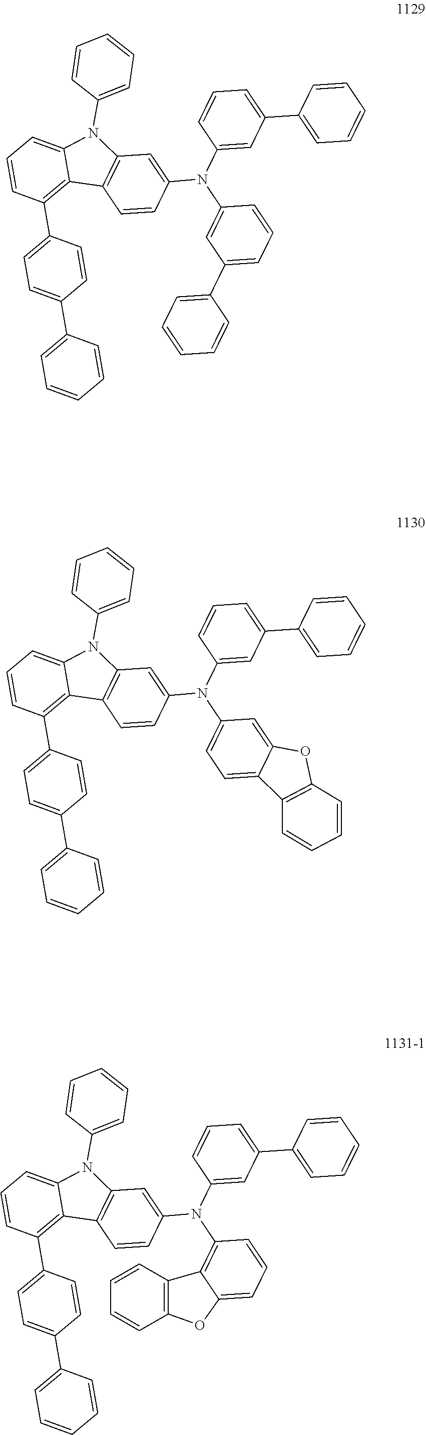 [SMILES: c1ccc(-c2ccc(-c3cccc4c3c3ccc(N(c5cccc(-c6ccccc6)c5)c5cccc(-c6ccccc6)c5)cc3n4-c3ccccc3)cc2)cc1 ; c1ccc(-c2ccc(-c3cccc4c3c3ccc(N(c5cccc(-c6ccccc6)c5)c5ccc6c(c5)oc5ccccc56)cc3n4-c3ccccc3)cc2)cc1 ; c1ccc(-c2ccc(-c3cccc4c3c3ccc(N(c5cccc(-c6ccccc6)c5)c5cccc6oc7ccccc7c56)cc3n4-c3ccccc3)cc2)cc1]

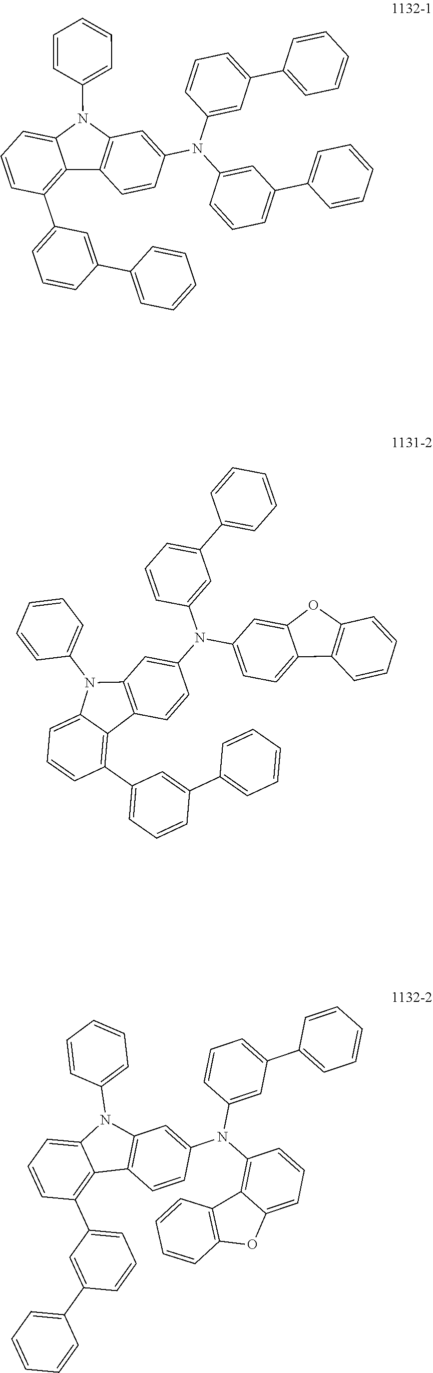 [SMILES: c1ccc(-c2cccc(-c3cccc4c3c3ccc(N(c5cccc(-c6ccccc6)c5)c5cccc(-c6ccccc6)c5)cc3n4-c3ccccc3)c2)cc1 ; c1ccc(-c2cccc(-c3cccc4c3c3ccc(N(c5cccc(-c6ccccc6)c5)c5ccc6c(c5)oc5ccccc56)cc3n4-c3ccccc3)c2)cc1 ; c1ccc(-c2cccc(-c3cccc4c3c3ccc(N(c5cccc(-c6ccccc6)c5)c5cccc6oc7ccccc7c56)cc3n4-c3ccccc3)c2)cc1]

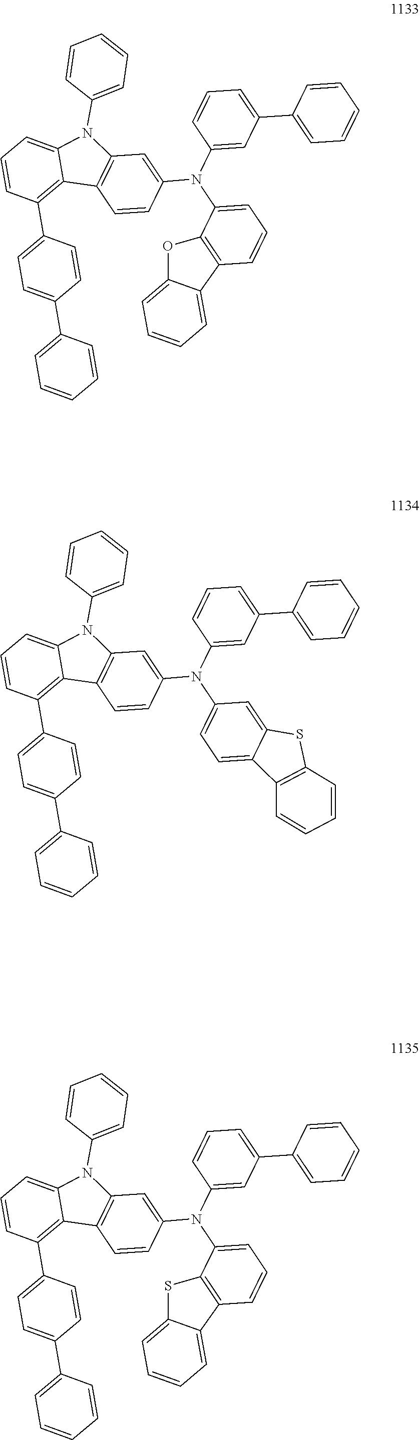 [SMILES: c1ccc(-c2ccc(-c3cccc4c3c3ccc(N(c5cccc(-c6ccccc6)c5)c5cccc6c5oc5ccccc56)cc3n4-c3ccccc3)cc2)cc1 ; c1ccc(-c2ccc(-c3cccc4c3c3ccc(N(c5cccc(-c6ccccc6)c5)c5ccc6c(c5)sc5ccccc56)cc3n4-c3ccccc3)cc2)cc1 ; c1ccc(-c2ccc(-c3cccc4c3c3ccc(N(c5cccc(-c6ccccc6)c5)c5cccc6c5sc5ccccc56)cc3n4-c3ccccc3)cc2)cc1]

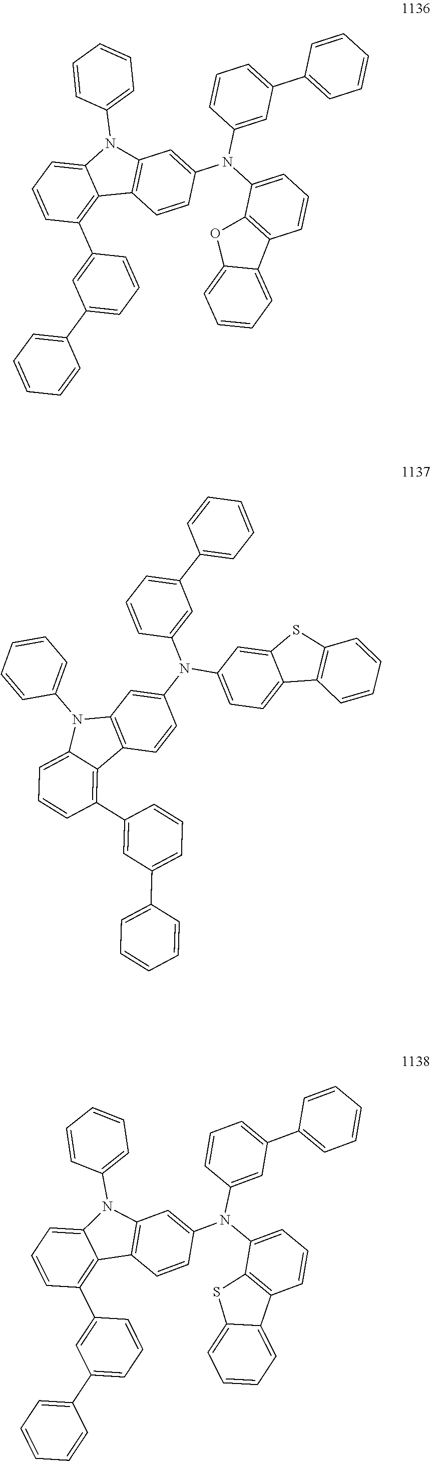 [SMILES: c1ccc(-c2cccc(-c3cccc4c3c3ccc(N(c5cccc(-c6ccccc6)c5)c5cccc6c5oc5ccccc56)cc3n4-c3ccccc3)c2)cc1 ; c1ccc(-c2cccc(-c3cccc4c3c3ccc(N(c5cccc(-c6ccccc6)c5)c5ccc6c(c5)sc5ccccc56)cc3n4-c3ccccc3)c2)cc1 ; c1ccc(-c2cccc(-c3cccc4c3c3ccc(N(c5cccc(-c6ccccc6)c5)c5cccc6c5sc5ccccc56)cc3n4-c3ccccc3)c2)cc1]

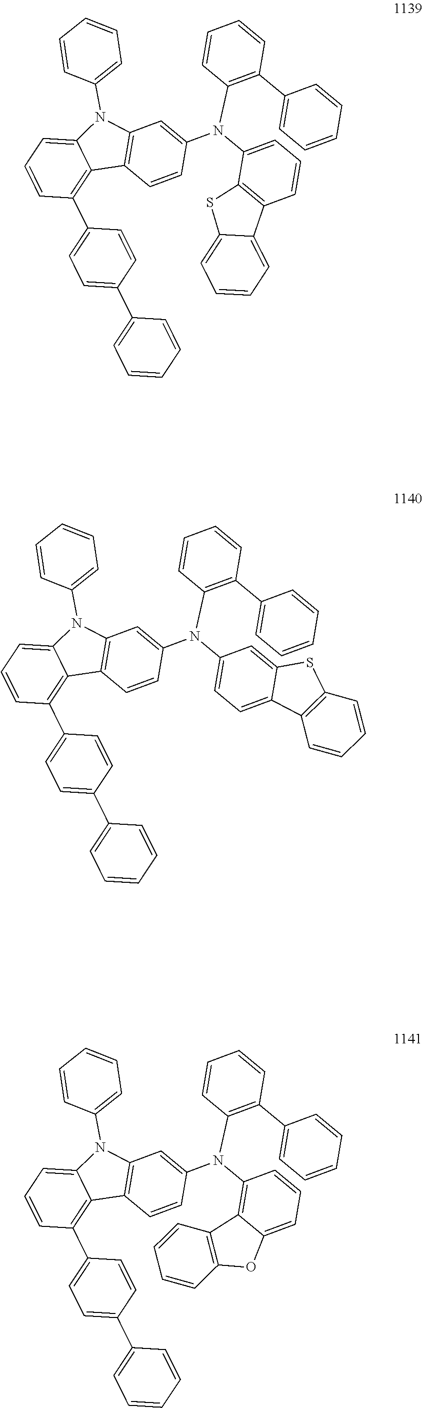 [SMILES: c1ccc(-c2ccc(-c3cccc4c3c3ccc(N(c5ccccc5-c5ccccc5)c5cccc6c5sc5ccccc56)cc3n4-c3ccccc3)cc2)cc1 ; c1ccc(-c2ccc(-c3cccc4c3c3ccc(N(c5ccc6c(c5)sc5ccccc56)c5ccccc5-c5ccccc5)cc3n4-c3ccccc3)cc2)cc1 ; c1ccc(-c2ccc(-c3cccc4c3c3ccc(N(c5ccccc5-c5ccccc5)c5cccc6oc7ccccc7c56)cc3n4-c3ccccc3)cc2)cc1]

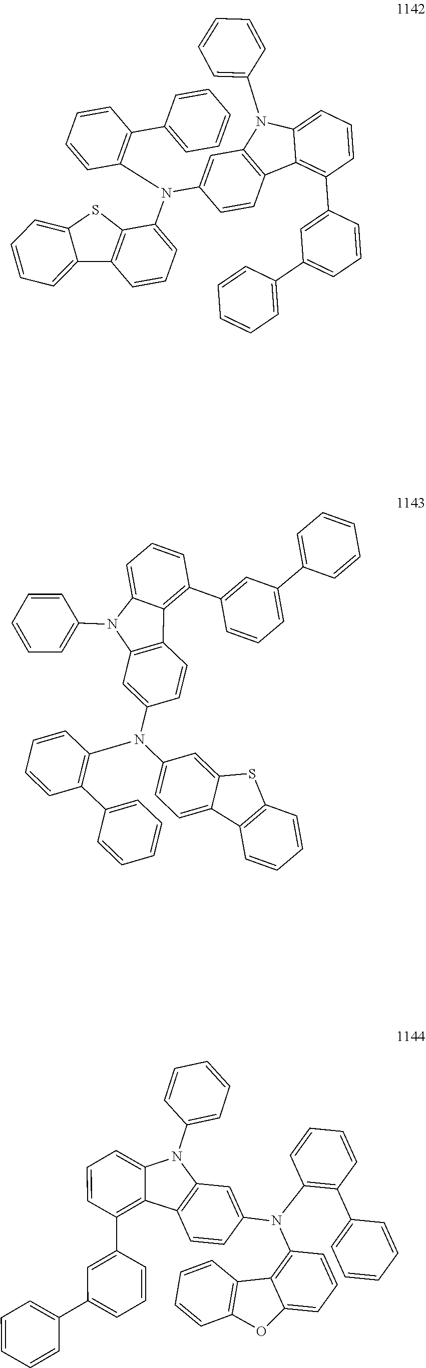 [SMILES: c1ccc(-c2cccc(-c3cccc4c3c3ccc(N(c5ccccc5-c5ccccc5)c5cccc6c5sc5ccccc56)cc3n4-c3ccccc3)c2)cc1 ; c1ccc(-c2cccc(-c3cccc4c3c3ccc(N(c5ccc6c(c5)sc5ccccc56)c5ccccc5-c5ccccc5)cc3n4-c3ccccc3)c2)cc1 ; c1ccc(-c2cccc(-c3cccc4c3c3ccc(N(c5ccccc5-c5ccccc5)c5cccc6oc7ccccc7c56)cc3n4-c3ccccc3)c2)cc1]

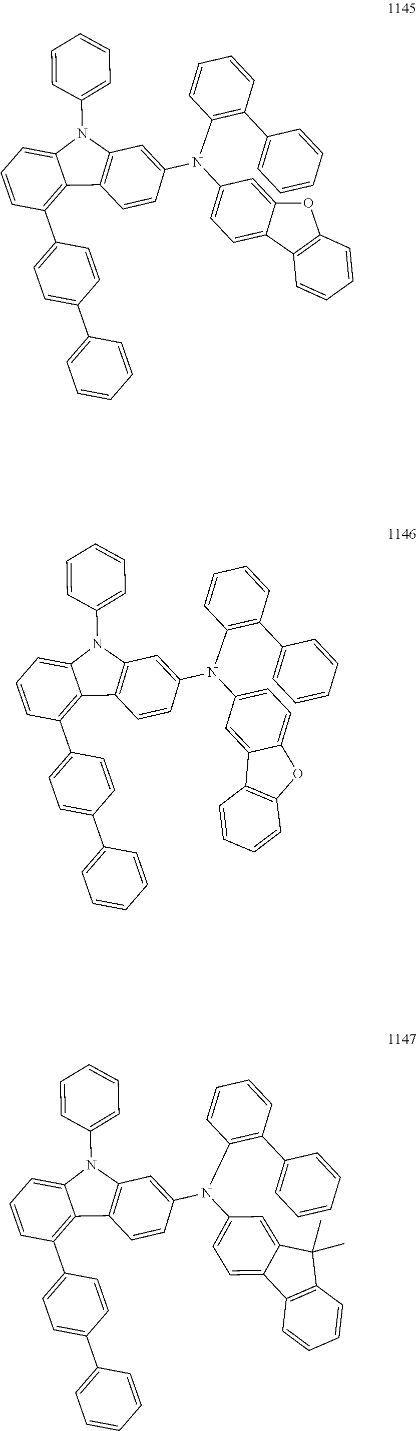 [SMILES: c1ccc(-c2ccc(-c3cccc4c3c3ccc(N(c5ccc6c(c5)oc5ccccc56)c5ccccc5-c5ccccc5)cc3n4-c3ccccc3)cc2)cc1 ; c1ccc(-c2ccc(-c3cccc4c3c3ccc(N(c5ccc6oc7ccccc7c6c5)c5ccccc5-c5ccccc5)cc3n4-c3ccccc3)cc2)cc1 ; CC1(C)c2ccccc2-c2ccc(N(c3ccc4c5c(-c6ccc(-c7ccccc7)cc6)cccc5n(-c5ccccc5)c4c3)c3ccccc3-c3ccccc3)cc21]

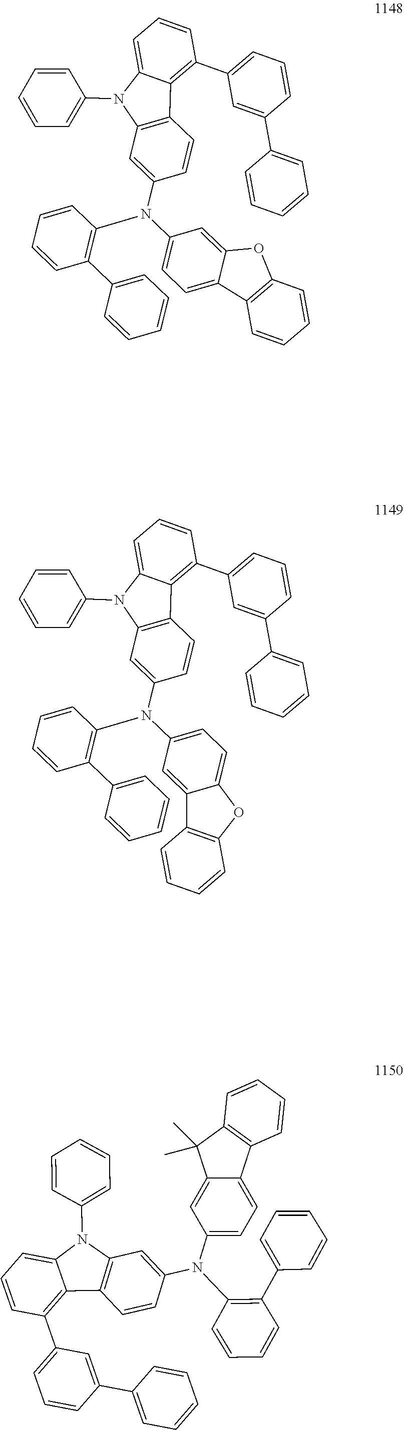 [SMILES: c1ccc(-c2cccc(-c3cccc4c3c3ccc(N(c5ccc6c(c5)oc5ccccc56)c5ccccc5-c5ccccc5)cc3n4-c3ccccc3)c2)cc1 ; c1ccc(-c2cccc(-c3cccc4c3c3ccc(N(c5ccc6oc7ccccc7c6c5)c5ccccc5-c5ccccc5)cc3n4-c3ccccc3)c2)cc1 ; CC1(C)c2ccccc2-c2ccc(N(c3ccc4c5c(-c6cccc(-c7ccccc7)c6)cccc5n(-c5ccccc5)c4c3)c3ccccc3-c3ccccc3)cc21]

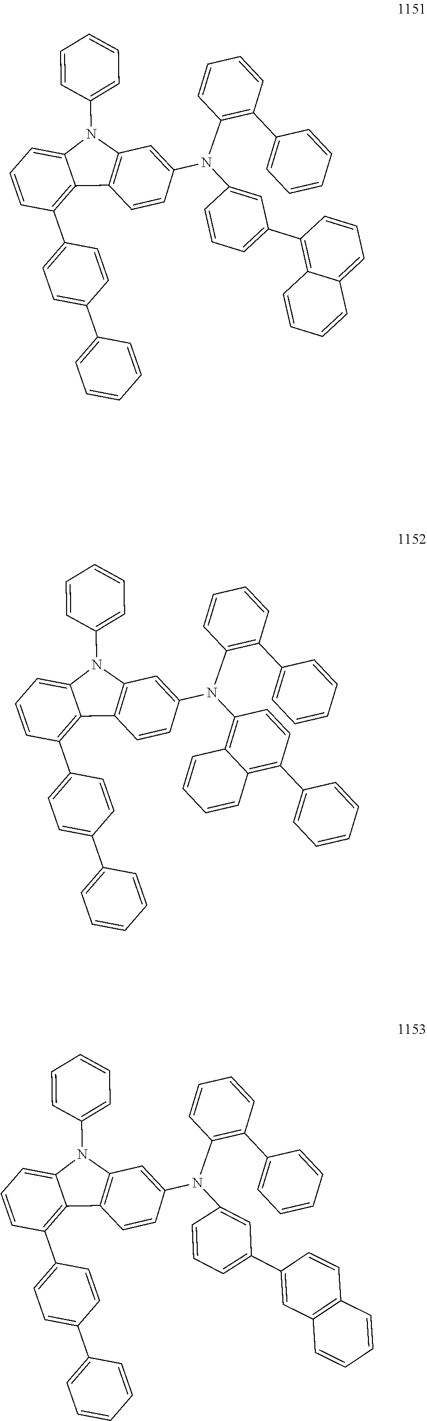 [SMILES: c1ccc(-c2ccc(-c3cccc4c3c3ccc(N(c5cccc(-c6cccc7ccccc67)c5)c5ccccc5-c5ccccc5)cc3n4-c3ccccc3)cc2)cc1 ; c1ccc(-c2ccc(-c3cccc4c3c3ccc(N(c5ccccc5-c5ccccc5)c5ccc(-c6ccccc6)c6ccccc56)cc3n4-c3ccccc3)cc2)cc1 ; c1ccc(-c2ccc(-c3cccc4c3c3ccc(N(c5cccc(-c6ccc7ccccc7c6)c5)c5ccccc5-c5ccccc5)cc3n4-c3ccccc3)cc2)cc1]

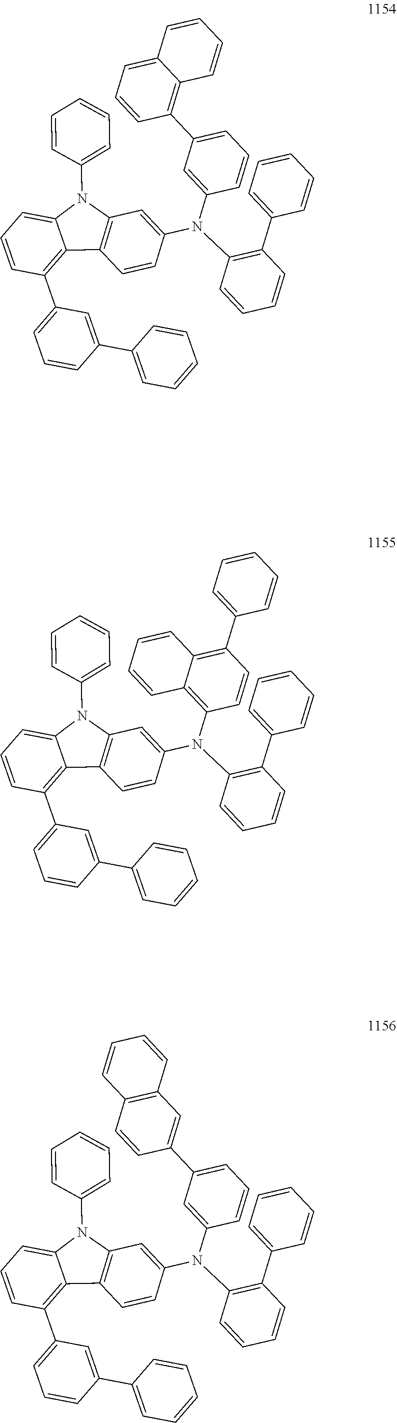 [SMILES: c1ccc(-c2cccc(-c3cccc4c3c3ccc(N(c5cccc(-c6cccc7ccccc67)c5)c5ccccc5-c5ccccc5)cc3n4-c3ccccc3)c2)cc1 ; c1ccc(-c2cccc(-c3cccc4c3c3ccc(N(c5ccccc5-c5ccccc5)c5ccc(-c6ccccc6)c6ccccc56)cc3n4-c3ccccc3)c2)cc1 ; c1ccc(-c2cccc(-c3cccc4c3c3ccc(N(c5cccc(-c6ccc7ccccc7c6)c5)c5ccccc5-c5ccccc5)cc3n4-c3ccccc3)c2)cc1]

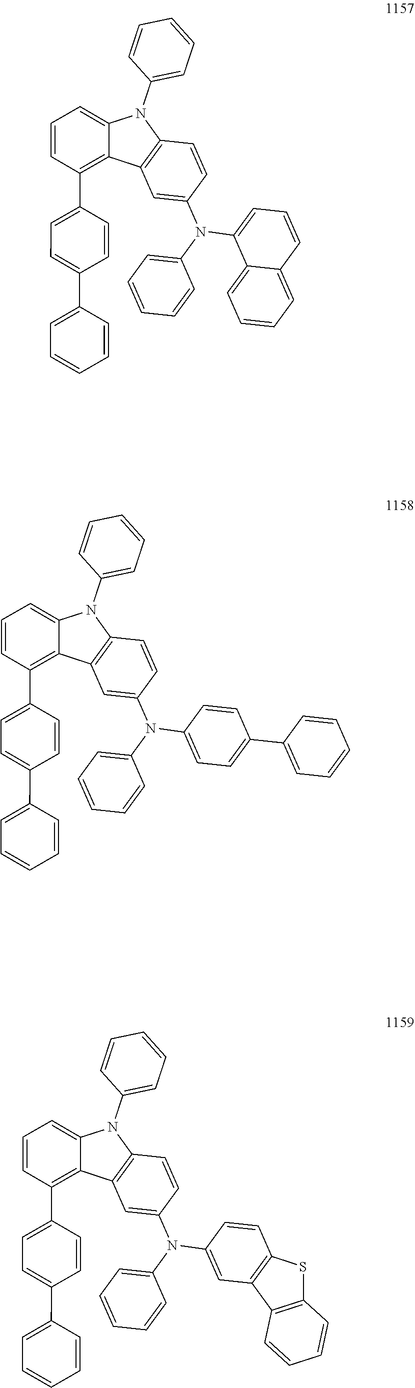 [SMILES: c1ccc(-c2ccc(-c3cccc4c3c3cc(N(c5ccccc5)c5cccc6ccccc56)ccc3n4-c3ccccc3)cc2)cc1 ; c1ccc(-c2ccc(-c3cccc4c3c3cc(N(c5ccccc5)c5ccc(-c6ccccc6)cc5)ccc3n4-c3ccccc3)cc2)cc1 ; c1ccc(-c2ccc(-c3cccc4c3c3cc(N(c5ccccc5)c5ccc6sc7ccccc7c6c5)ccc3n4-c3ccccc3)cc2)cc1]

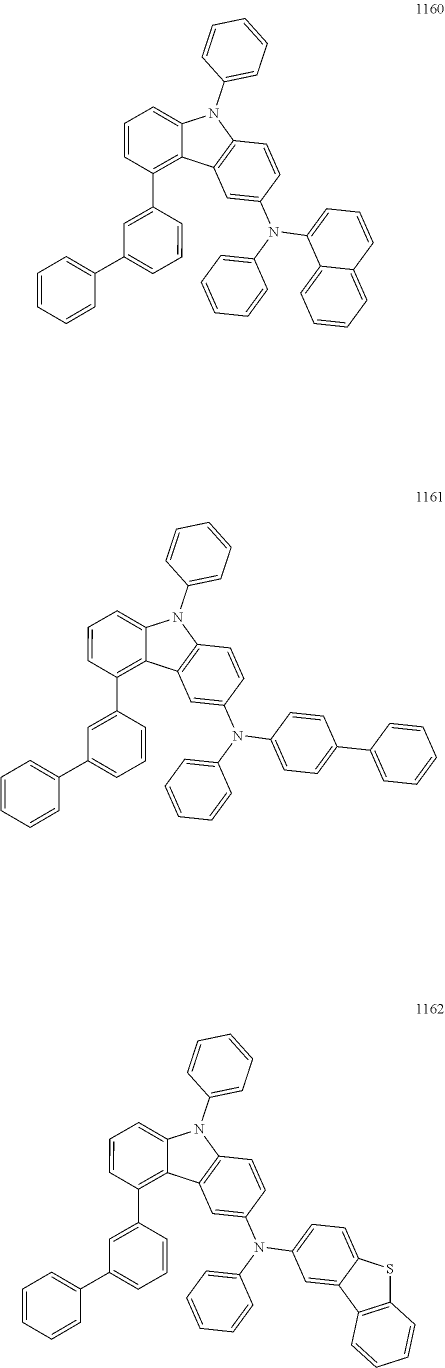 [SMILES: c1ccc(-c2cccc(-c3cccc4c3c3cc(N(c5ccccc5)c5cccc6ccccc56)ccc3n4-c3ccccc3)c2)cc1 ; c1ccc(-c2ccc(N(c3ccccc3)c3ccc4c(c3)c3c(-c5cccc(-c6ccccc6)c5)cccc3n4-c3ccccc3)cc2)cc1 ; c1ccc(-c2cccc(-c3cccc4c3c3cc(N(c5ccccc5)c5ccc6sc7ccccc7c6c5)ccc3n4-c3ccccc3)c2)cc1]

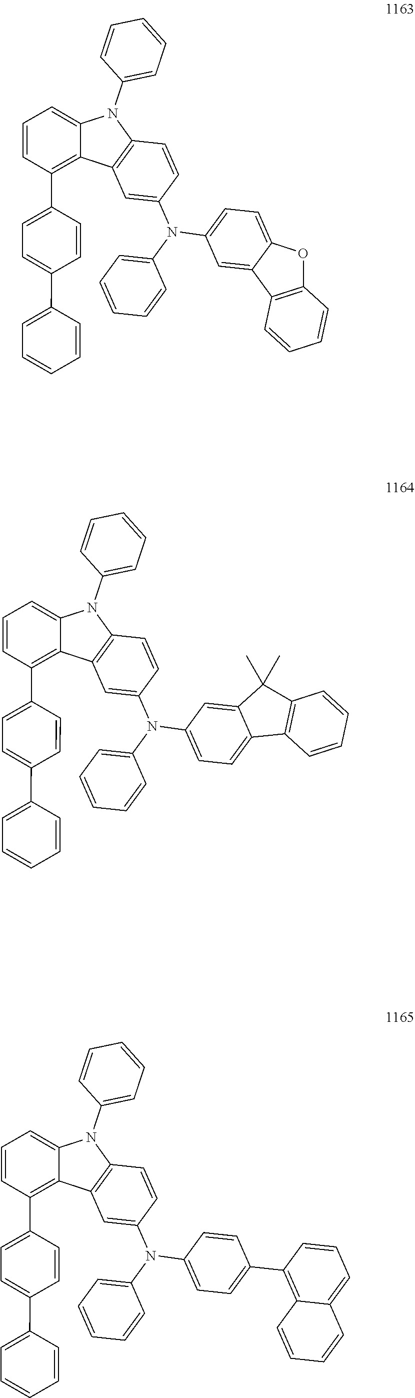 [SMILES: c1ccc(-c2ccc(-c3cccc4c3c3cc(N(c5ccccc5)c5ccc6oc7ccccc7c6c5)ccc3n4-c3ccccc3)cc2)cc1 ; CC1(C)c2ccccc2-c2ccc(N(c3ccccc3)c3ccc4c(c3)c3c(-c5ccc(-c6ccccc6)cc5)cccc3n4-c3ccccc3)cc21 ; c1ccc(-c2ccc(-c3cccc4c3c3cc(N(c5ccccc5)c5ccc(-c6cccc7ccccc67)cc5)ccc3n4-c3ccccc3)cc2)cc1]

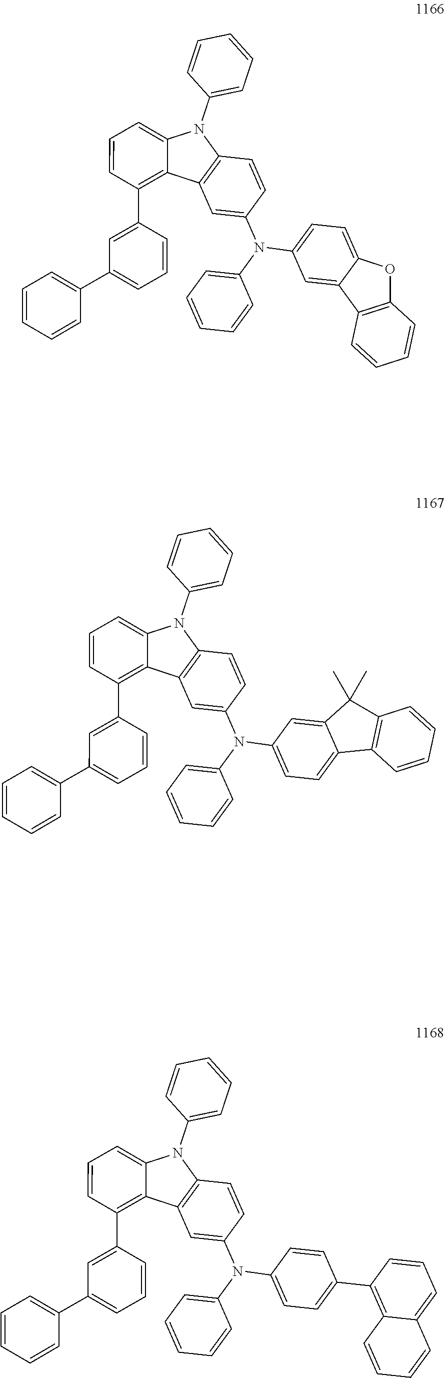 [SMILES: c1ccc(-c2cccc(-c3cccc4c3c3cc(N(c5ccccc5)c5ccc6oc7ccccc7c6c5)ccc3n4-c3ccccc3)c2)cc1 ; CC1(C)c2ccccc2-c2ccc(N(c3ccccc3)c3ccc4c(c3)c3c(-c5cccc(-c6ccccc6)c5)cccc3n4-c3ccccc3)cc21 ; c1ccc(-c2cccc(-c3cccc4c3c3cc(N(c5ccccc5)c5ccc(-c6cccc7ccccc67)cc5)ccc3n4-c3ccccc3)c2)cc1]

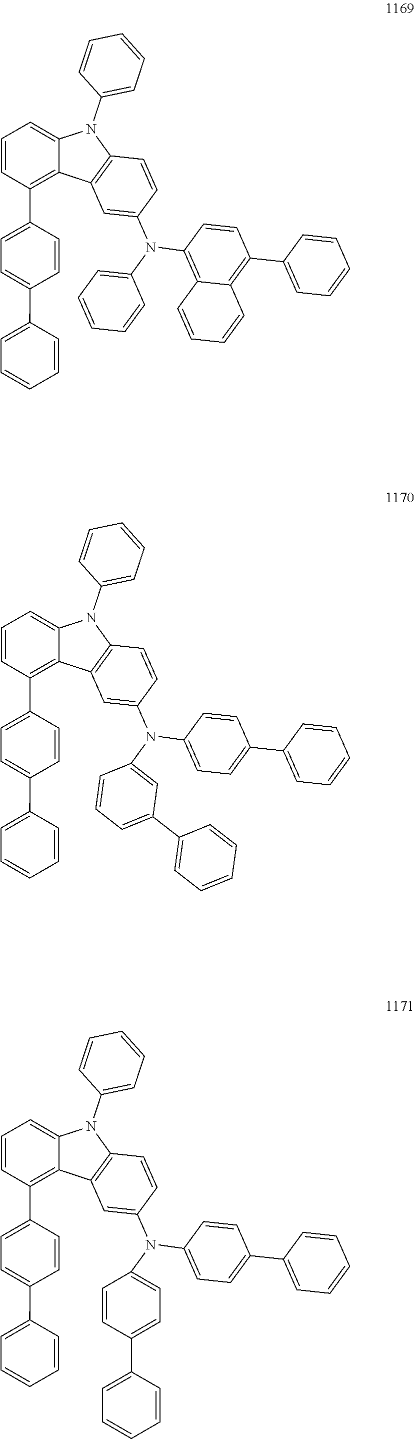 [SMILES: c1ccc(-c2ccc(-c3cccc4c3c3cc(N(c5ccccc5)c5ccc(-c6ccccc6)c6ccccc56)ccc3n4-c3ccccc3)cc2)cc1 ; c1ccc(-c2ccc(-c3cccc4c3c3cc(N(c5ccc(-c6ccccc6)cc5)c5cccc(-c6ccccc6)c5)ccc3n4-c3ccccc3)cc2)cc1 ; c1ccc(-c2ccc(-c3cccc4c3c3cc(N(c5ccc(-c6ccccc6)cc5)c5ccc(-c6ccccc6)cc5)ccc3n4-c3ccccc3)cc2)cc1]

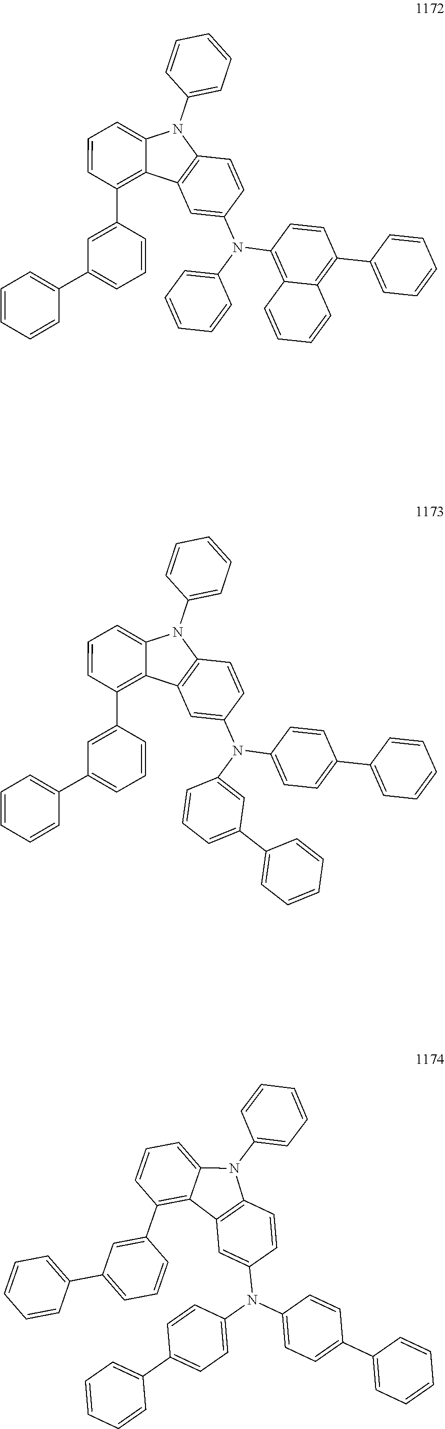 [SMILES: c1ccc(-c2cccc(-c3cccc4c3c3cc(N(c5ccccc5)c5ccc(-c6ccccc6)c6ccccc56)ccc3n4-c3ccccc3)c2)cc1 ; c1ccc(-c2ccc(N(c3cccc(-c4ccccc4)c3)c3ccc4c(c3)c3c(-c5cccc(-c6ccccc6)c5)cccc3n4-c3ccccc3)cc2)cc1 ; c1ccc(-c2ccc(N(c3ccc(-c4ccccc4)cc3)c3ccc4c(c3)c3c(-c5cccc(-c6ccccc6)c5)cccc3n4-c3ccccc3)cc2)cc1]

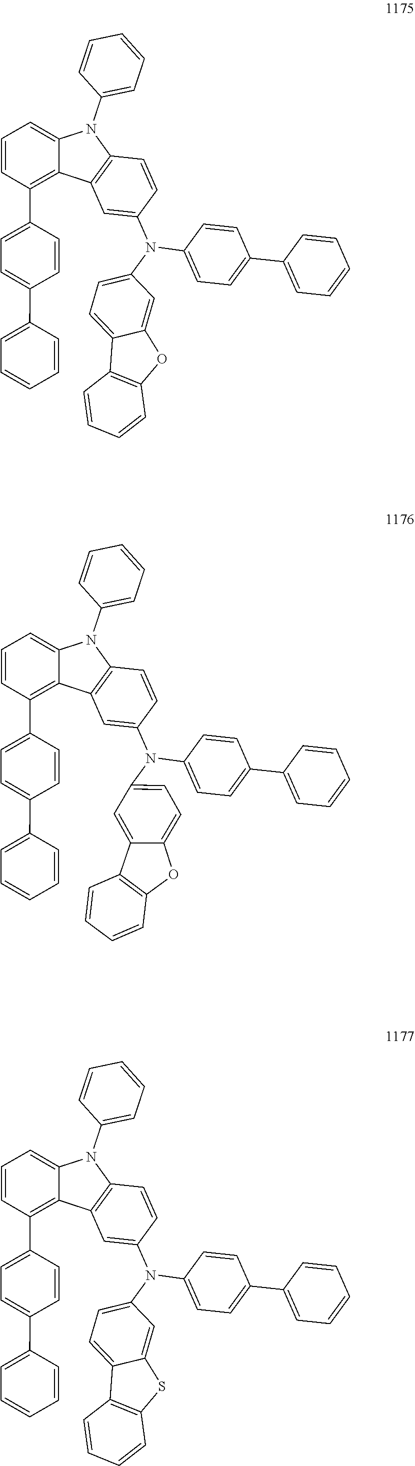 [SMILES: c1ccc(-c2ccc(-c3cccc4c3c3cc(N(c5ccc(-c6ccccc6)cc5)c5ccc6c(c5)oc5ccccc56)ccc3n4-c3ccccc3)cc2)cc1 ; c1ccc(-c2ccc(-c3cccc4c3c3cc(N(c5ccc(-c6ccccc6)cc5)c5ccc6oc7ccccc7c6c5)ccc3n4-c3ccccc3)cc2)cc1 ; c1ccc(-c2ccc(-c3cccc4c3c3cc(N(c5ccc(-c6ccccc6)cc5)c5ccc6c(c5)sc5ccccc56)ccc3n4-c3ccccc3)cc2)cc1]

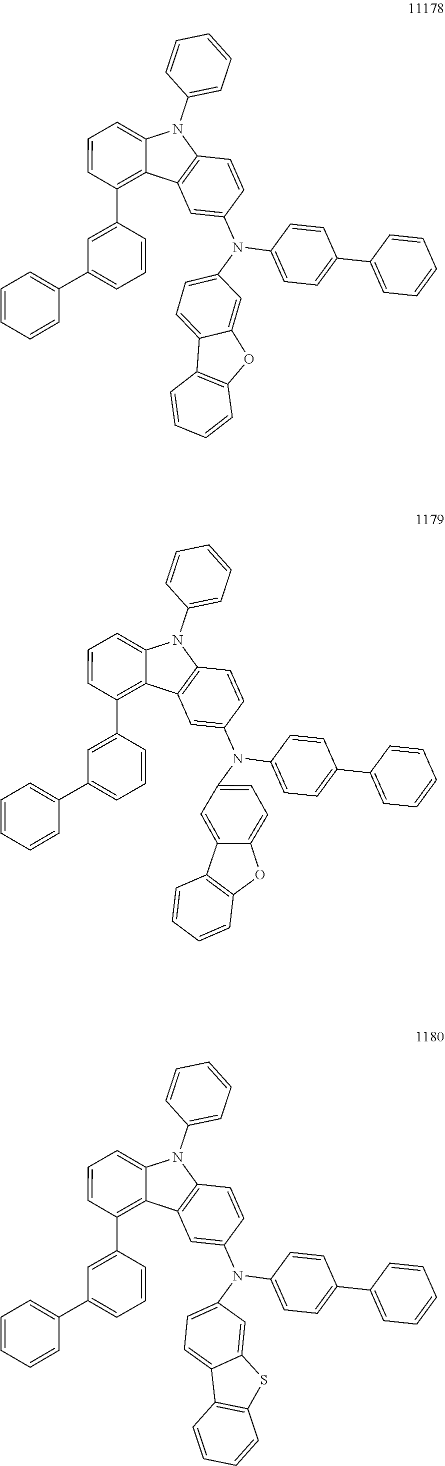 [SMILES: c1ccc(-c2ccc(N(c3ccc4c(c3)oc3ccccc34)c3ccc4c(c3)c3c(-c5cccc(-c6ccccc6)c5)cccc3n4-c3ccccc3)cc2)cc1 ; c1ccc(-c2ccc(N(c3ccc4oc5ccccc5c4c3)c3ccc4c(c3)c3c(-c5cccc(-c6ccccc6)c5)cccc3n4-c3ccccc3)cc2)cc1 ; c1ccc(-c2ccc(N(c3ccc4c(c3)sc3ccccc34)c3ccc4c(c3)c3c(-c5cccc(-c6ccccc6)c5)cccc3n4-c3ccccc3)cc2)cc1]

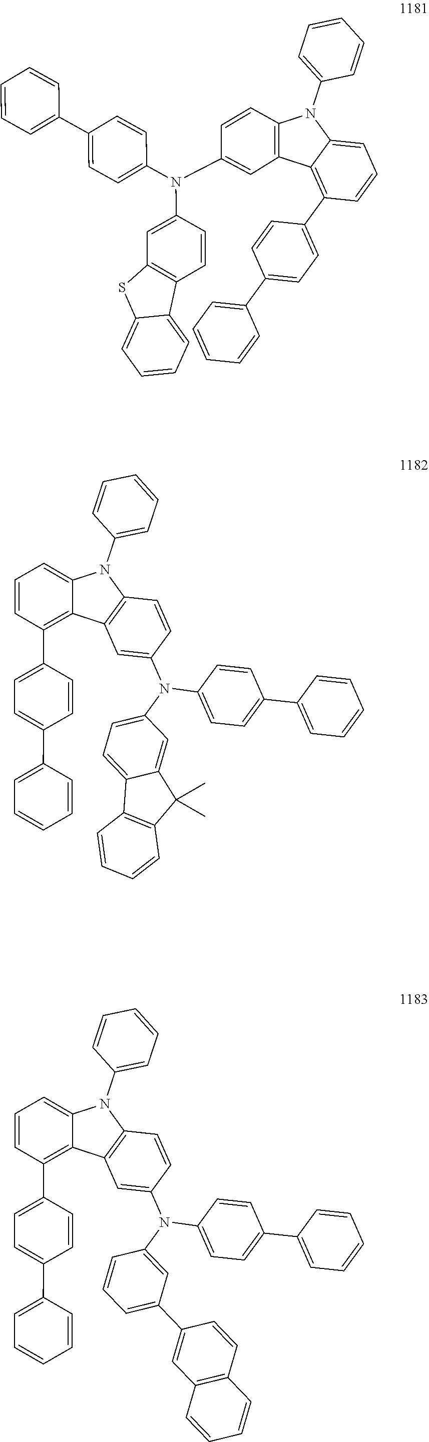 [SMILES: c1ccc(-c2ccc(-c3cccc4c3c3cc(N(c5ccc(-c6ccccc6)cc5)c5ccc6c(c5)sc5ccccc56)ccc3n4-c3ccccc3)cc2)cc1 ; CC1(C)c2ccccc2-c2ccc(N(c3ccc(-c4ccccc4)cc3)c3ccc4c(c3)c3c(-c5ccc(-c6ccccc6)cc5)cccc3n4-c3ccccc3)cc21 ; c1ccc(-c2ccc(-c3cccc4c3c3cc(N(c5ccc(-c6ccccc6)cc5)c5cccc(-c6ccc7ccccc7c6)c5)ccc3n4-c3ccccc3)cc2)cc1]

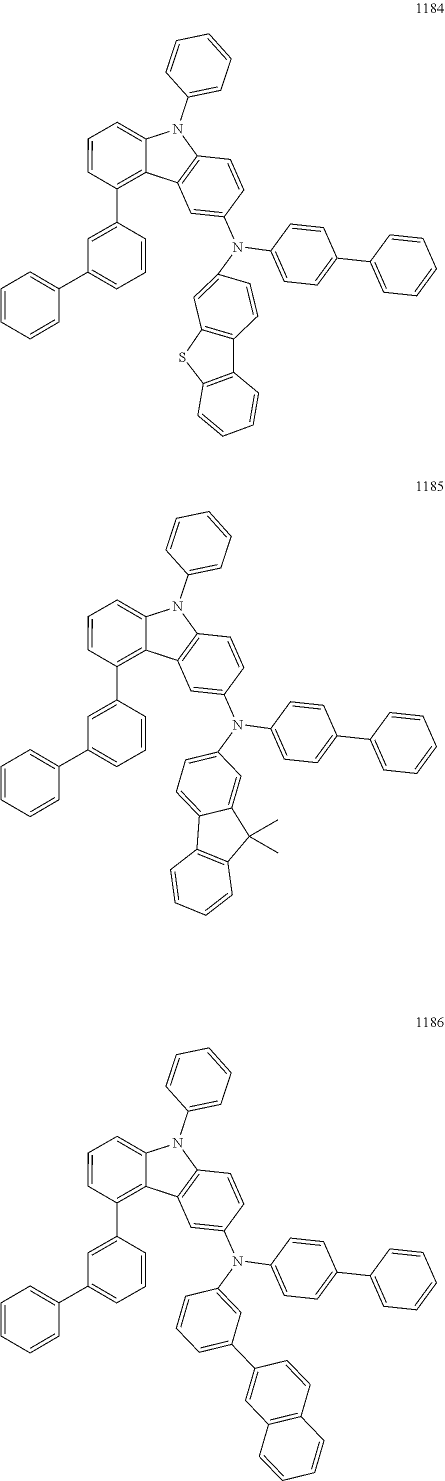 [SMILES: c1ccc(-c2ccc(N(c3ccc4c(c3)sc3ccccc34)c3ccc4c(c3)c3c(-c5cccc(-c6ccccc6)c5)cccc3n4-c3ccccc3)cc2)cc1 ; CC1(C)c2ccccc2-c2ccc(N(c3ccc(-c4ccccc4)cc3)c3ccc4c(c3)c3c(-c5cccc(-c6ccccc6)c5)cccc3n4-c3ccccc3)cc21 ; c1ccc(-c2ccc(N(c3cccc(-c4ccc5ccccc5c4)c3)c3ccc4c(c3)c3c(-c5cccc(-c6ccccc6)c5)cccc3n4-c3ccccc3)cc2)cc1]

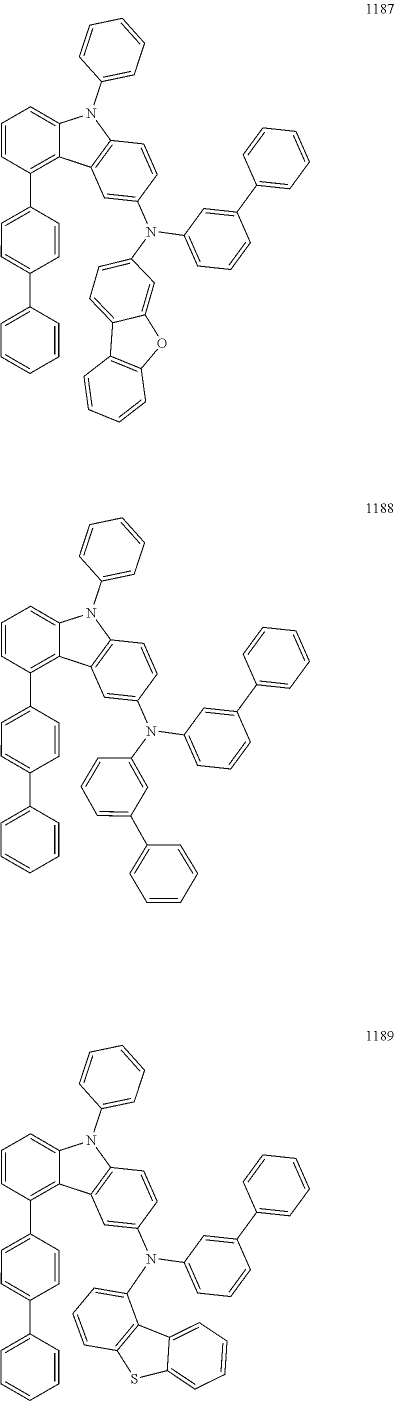 [SMILES: c1ccc(-c2ccc(-c3cccc4c3c3cc(N(c5cccc(-c6ccccc6)c5)c5ccc6c(c5)oc5ccccc56)ccc3n4-c3ccccc3)cc2)cc1 ; c1ccc(-c2ccc(-c3cccc4c3c3cc(N(c5cccc(-c6ccccc6)c5)c5cccc(-c6ccccc6)c5)ccc3n4-c3ccccc3)cc2)cc1 ; c1ccc(-c2ccc(-c3cccc4c3c3cc(N(c5cccc(-c6ccccc6)c5)c5cccc6sc7ccccc7c56)ccc3n4-c3ccccc3)cc2)cc1]

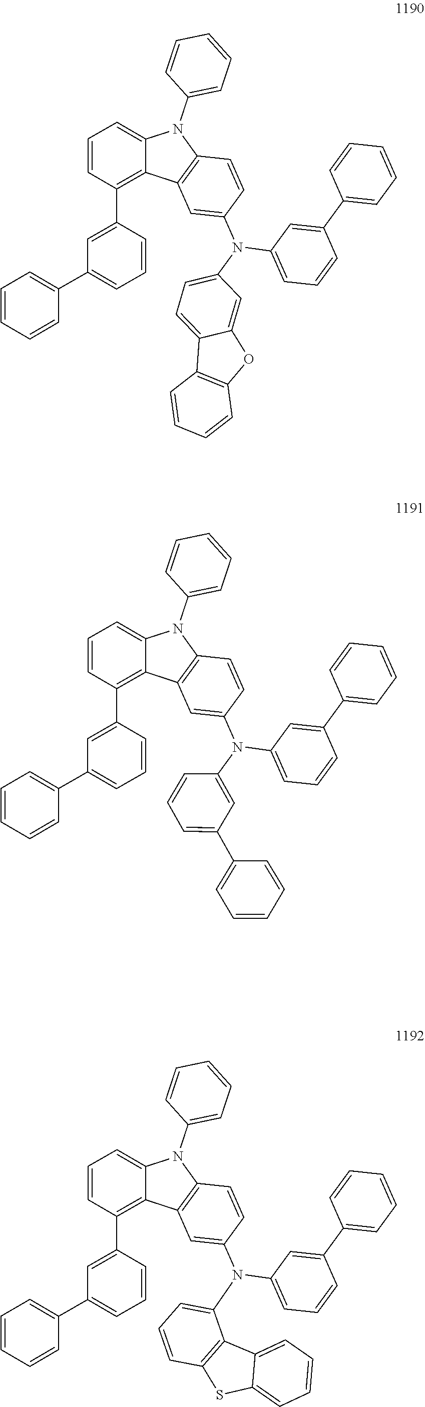 [SMILES: c1ccc(-c2cccc(-c3cccc4c3c3cc(N(c5cccc(-c6ccccc6)c5)c5ccc6c(c5)oc5ccccc56)ccc3n4-c3ccccc3)c2)cc1 ; c1ccc(-c2cccc(-c3cccc4c3c3cc(N(c5cccc(-c6ccccc6)c5)c5cccc(-c6ccccc6)c5)ccc3n4-c3ccccc3)c2)cc1 ; c1ccc(-c2cccc(-c3cccc4c3c3cc(N(c5cccc(-c6ccccc6)c5)c5cccc6sc7ccccc7c56)ccc3n4-c3ccccc3)c2)cc1]

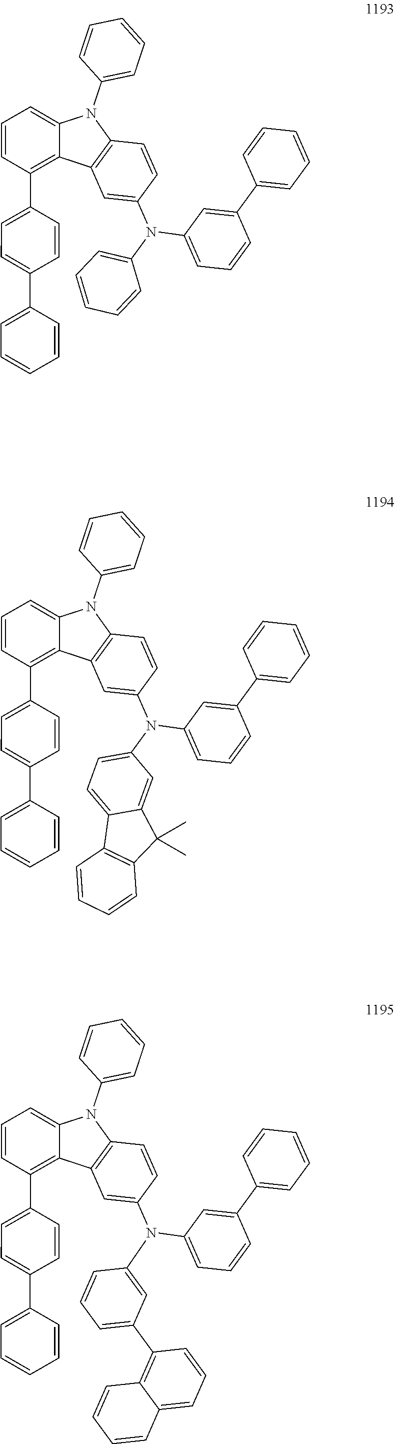 [SMILES: c1ccc(-c2ccc(-c3cccc4c3c3cc(N(c5ccccc5)c5cccc(-c6ccccc6)c5)ccc3n4-c3ccccc3)cc2)cc1 ; CC1(C)c2ccccc2-c2ccc(N(c3cccc(-c4ccccc4)c3)c3ccc4c(c3)c3c(-c5ccc(-c6ccccc6)cc5)cccc3n4-c3ccccc3)cc21 ; c1ccc(-c2ccc(-c3cccc4c3c3cc(N(c5cccc(-c6ccccc6)c5)c5cccc(-c6cccc7ccccc67)c5)ccc3n4-c3ccccc3)cc2)cc1]

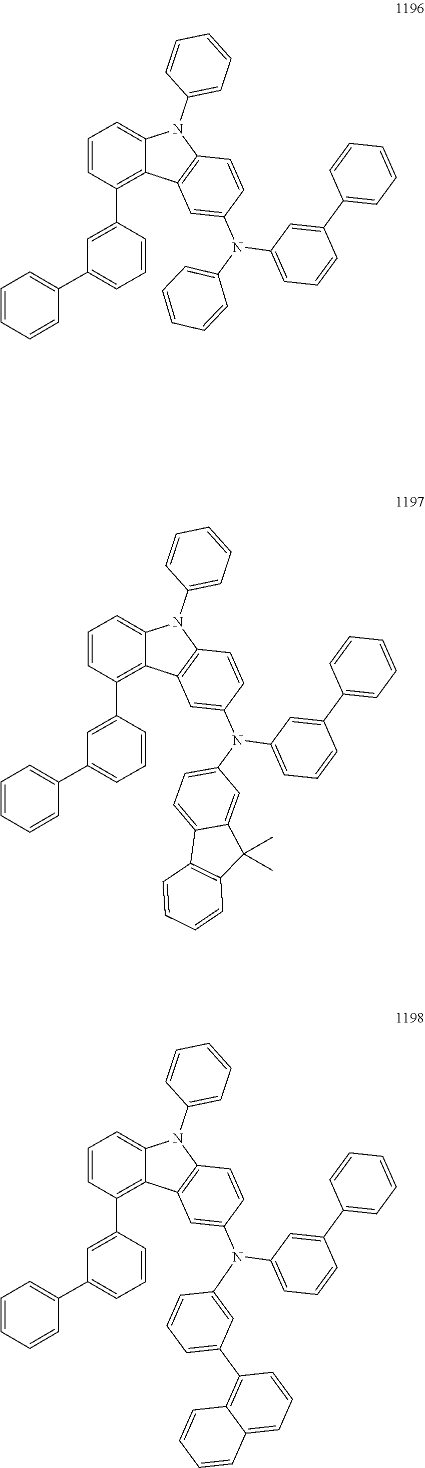 [SMILES: c1ccc(-c2cccc(-c3cccc4c3c3cc(N(c5ccccc5)c5cccc(-c6ccccc6)c5)ccc3n4-c3ccccc3)c2)cc1 ; CC1(C)c2ccccc2-c2ccc(N(c3cccc(-c4ccccc4)c3)c3ccc4c(c3)c3c(-c5cccc(-c6ccccc6)c5)cccc3n4-c3ccccc3)cc21 ; c1ccc(-c2cccc(-c3cccc4c3c3cc(N(c5cccc(-c6ccccc6)c5)c5cccc(-c6cccc7ccccc67)c5)ccc3n4-c3ccccc3)c2)cc1]

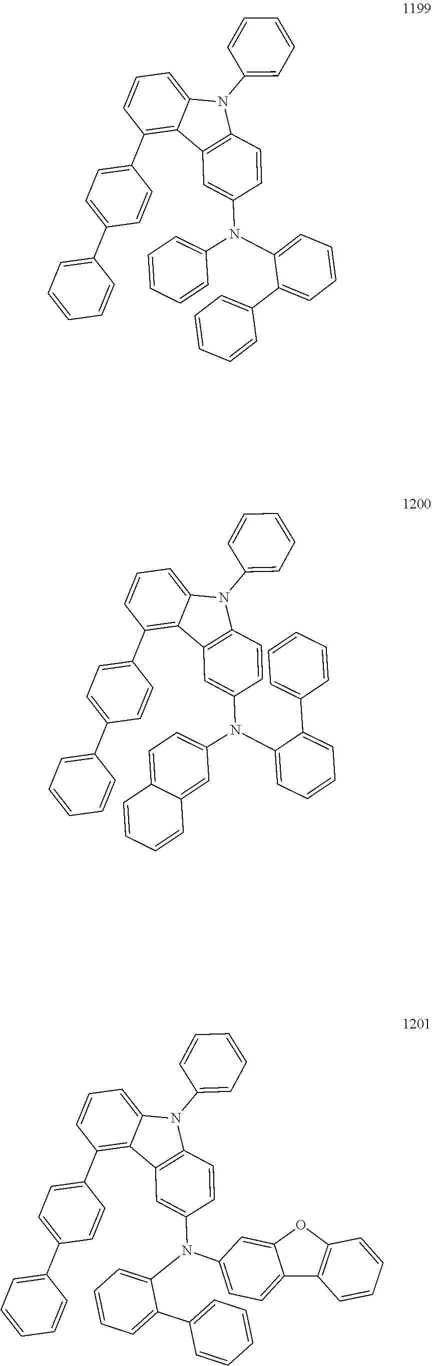 [SMILES: c1ccc(-c2ccc(-c3cccc4c3c3cc(N(c5ccccc5)c5ccccc5-c5ccccc5)ccc3n4-c3ccccc3)cc2)cc1 ; c1ccc(-c2ccc(-c3cccc4c3c3cc(N(c5ccc6ccccc6c5)c5ccccc5-c5ccccc5)ccc3n4-c3ccccc3)cc2)cc1 ; c1ccc(-c2ccc(-c3cccc4c3c3cc(N(c5ccc6c(c5)oc5ccccc56)c5ccccc5-c5ccccc5)ccc3n4-c3ccccc3)cc2)cc1]

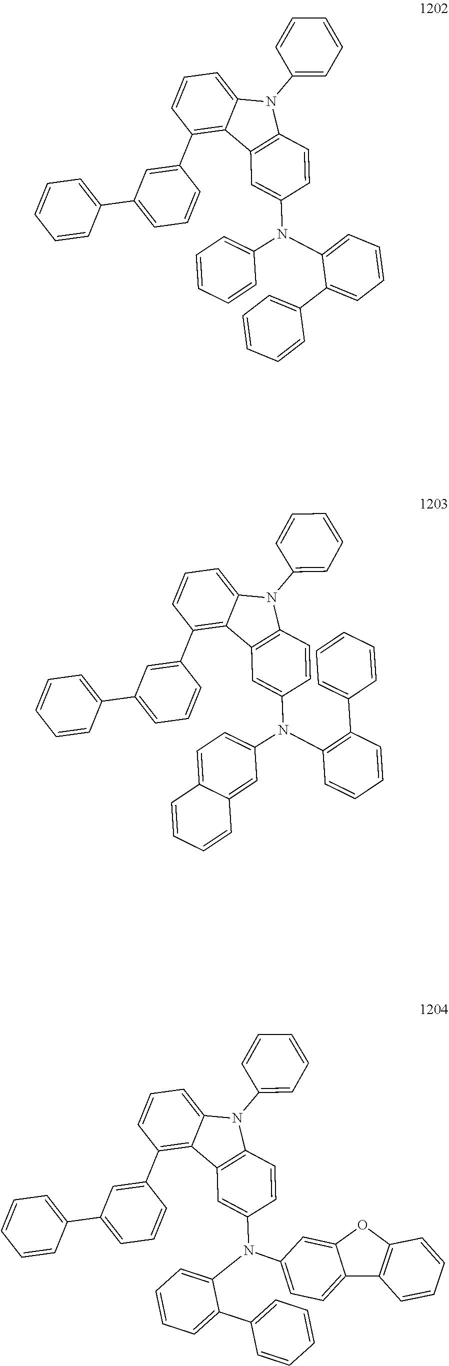 [SMILES: c1ccc(-c2cccc(-c3cccc4c3c3cc(N(c5ccccc5)c5ccccc5-c5ccccc5)ccc3n4-c3ccccc3)c2)cc1 ; c1ccc(-c2cccc(-c3cccc4c3c3cc(N(c5ccc6ccccc6c5)c5ccccc5-c5ccccc5)ccc3n4-c3ccccc3)c2)cc1 ; c1ccc(-c2cccc(-c3cccc4c3c3cc(N(c5ccc6c(c5)oc5ccccc56)c5ccccc5-c5ccccc5)ccc3n4-c3ccccc3)c2)cc1]

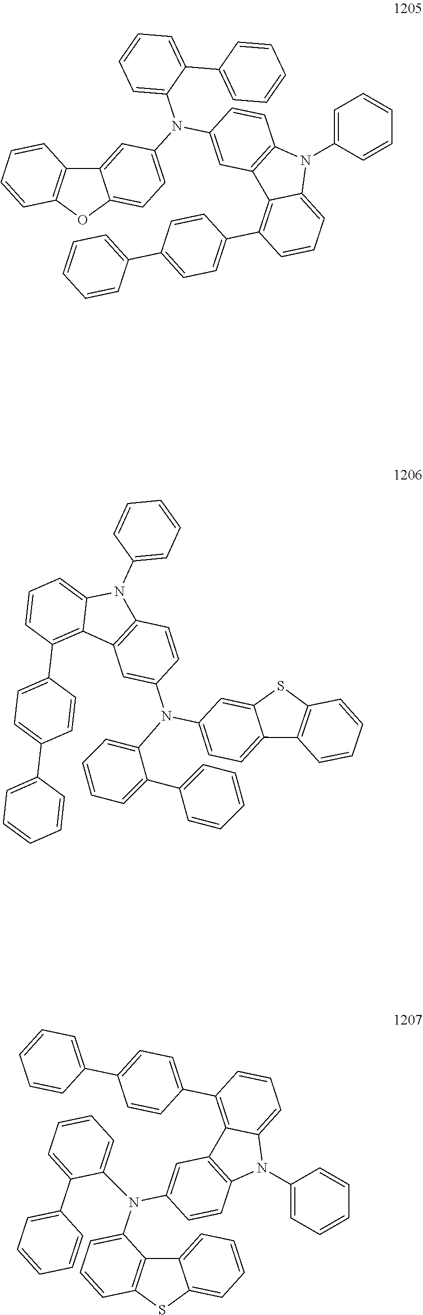 [SMILES: c1ccc(-c2ccc(-c3cccc4c3c3cc(N(c5ccc6oc7ccccc7c6c5)c5ccccc5-c5ccccc5)ccc3n4-c3ccccc3)cc2)cc1 ; c1ccc(-c2ccc(-c3cccc4c3c3cc(N(c5ccc6c(c5)sc5ccccc56)c5ccccc5-c5ccccc5)ccc3n4-c3ccccc3)cc2)cc1 ; c1ccc(-c2ccc(-c3cccc4c3c3cc(N(c5ccccc5-c5ccccc5)c5cccc6sc7ccccc7c56)ccc3n4-c3ccccc3)cc2)cc1]

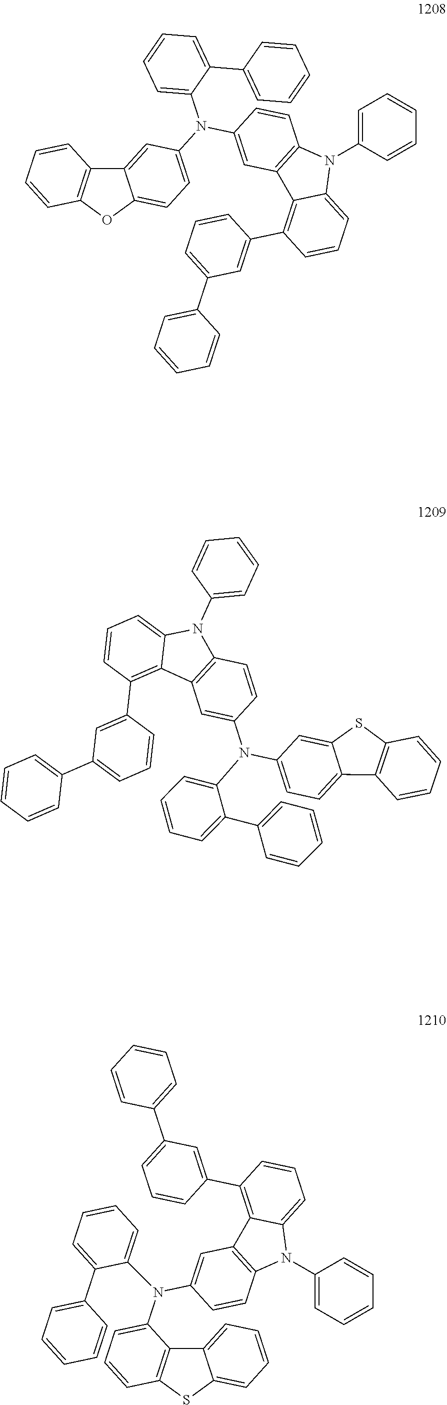 [SMILES: c1ccc(-c2cccc(-c3cccc4c3c3cc(N(c5ccc6oc7ccccc7c6c5)c5ccccc5-c5ccccc5)ccc3n4-c3ccccc3)c2)cc1 ; c1ccc(-c2cccc(-c3cccc4c3c3cc(N(c5ccc6c(c5)sc5ccccc56)c5ccccc5-c5ccccc5)ccc3n4-c3ccccc3)c2)cc1 ; c1ccc(-c2cccc(-c3cccc4c3c3cc(N(c5ccccc5-c5ccccc5)c5cccc6sc7ccccc7c56)ccc3n4-c3ccccc3)c2)cc1]

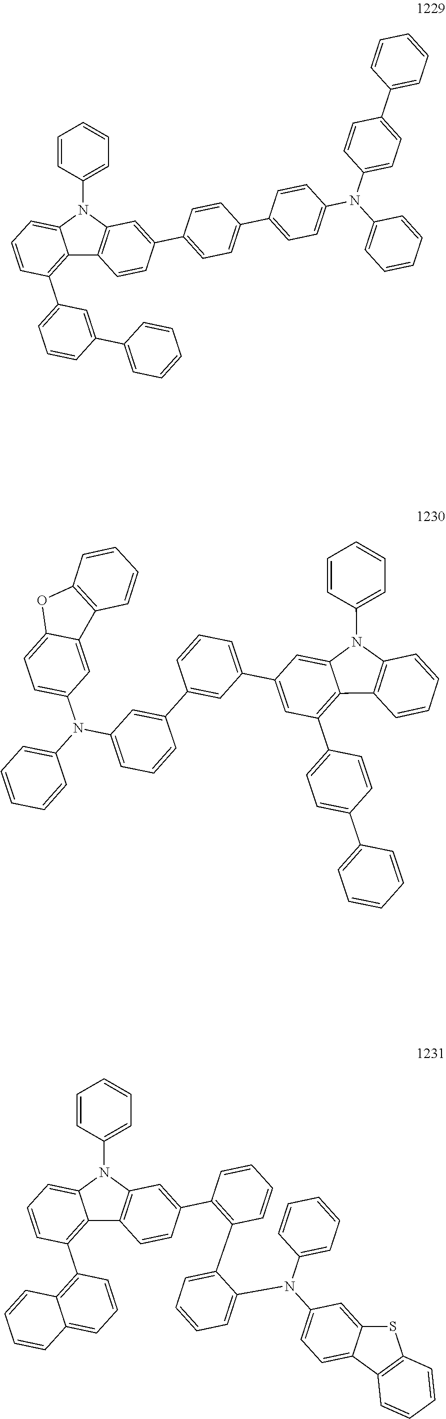 [SMILES: c1ccc(-c2ccc(N(c3ccccc3)c3ccc(-c4ccc(-c5ccc6c7c(-c8cccc(-c9ccccc9)c8)cccc7n(-c7ccccc7)c6c5)cc4)cc3)cc2)cc1 ; c1ccc(-c2ccc(-c3cc(-c4cccc(-c5cccc(N(c6ccccc6)c6ccc7oc8ccccc8c7c6)c5)c4)cc4c3c3ccccc3n4-c3ccccc3)cc2)cc1 ; c1ccc(N(c2ccc3c(c2)sc2ccccc23)c2ccccc2-c2ccccc2-c2ccc3c4c(-c5cccc6ccccc56)cccc4n(-c4ccccc4)c3c2)cc1]

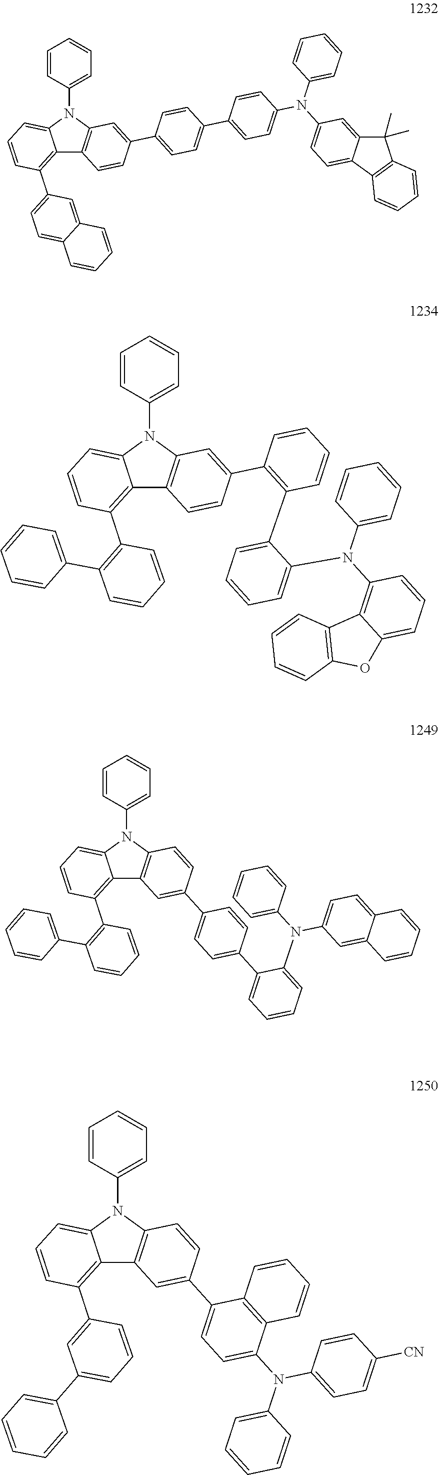 [SMILES: CC1(C)c2ccccc2-c2ccc(N(c3ccccc3)c3ccc(-c4ccc(-c5ccc6c7c(-c8ccc9ccccc9c8)cccc7n(-c7ccccc7)c6c5)cc4)cc3)cc21 ; c1ccc(-c2ccccc2-c2cccc3c2c2ccc(-c4ccccc4-c4ccccc4N(c4ccccc4)c4cccc5oc6ccccc6c45)cc2n3-c2ccccc2)cc1 ; c1ccc(-c2ccccc2-c2cccc3c2c2cc(-c4ccc(-c5ccccc5N(c5ccccc5)c5ccc6ccccc6c5)cc4)ccc2n3-c2ccccc2)cc1 ; N#Cc1ccc(N(c2ccccc2)c2ccc(-c3ccc4c(c3)c3c(-c5cccc(-c6ccccc6)c5)cccc3n4-c3ccccc3)c3ccccc23)cc1]

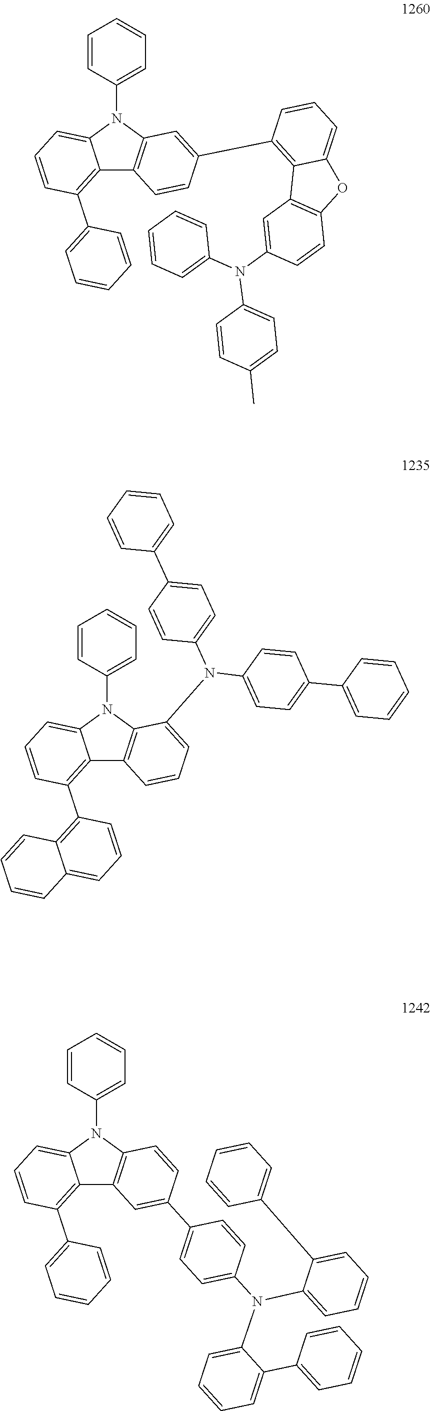 [SMILES: Cc1ccc(N(c2ccccc2)c2ccc3oc4cccc(-c5ccc6c7c(-c8ccccc8)cccc7n(-c7ccccc7)c6c5)c4c3c2)cc1 ; c1ccc(-c2ccc(N(c3ccc(-c4ccccc4)cc3)c3cccc4c5c(-c6cccc7ccccc67)cccc5n(-c5ccccc5)c34)cc2)cc1 ; c1ccc(-c2ccccc2N(c2ccc(-c3ccc4c(c3)c3c(-c5ccccc5)cccc3n4-c3ccccc3)cc2)c2ccccc2-c2ccccc2)cc1]

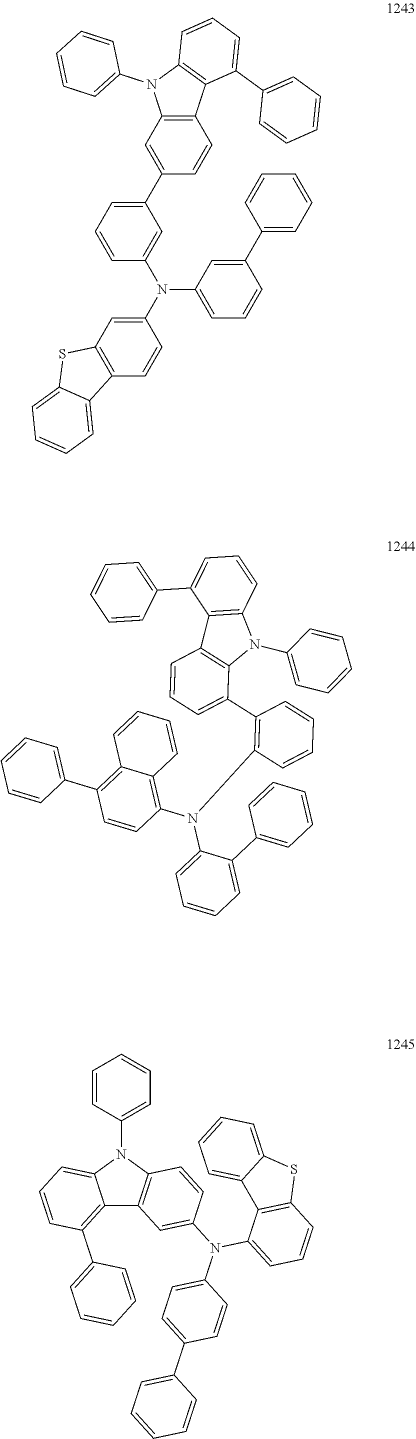 [SMILES: c1ccc(-c2cccc(N(c3cccc(-c4ccc5c6c(-c7ccccc7)cccc6n(-c6ccccc6)c5c4)c3)c3ccc4c(c3)sc3ccccc34)c2)cc1 ; c1ccc(-c2ccccc2N(c2ccccc2-c2cccc3c4c(-c5ccccc5)cccc4n(-c4ccccc4)c23)c2ccc(-c3ccccc3)c3ccccc23)cc1 ; c1ccc(-c2ccc(N(c3ccc4c(c3)c3c(-c5ccccc5)cccc3n4-c3ccccc3)c3cccc4sc5ccccc5c34)cc2)cc1]

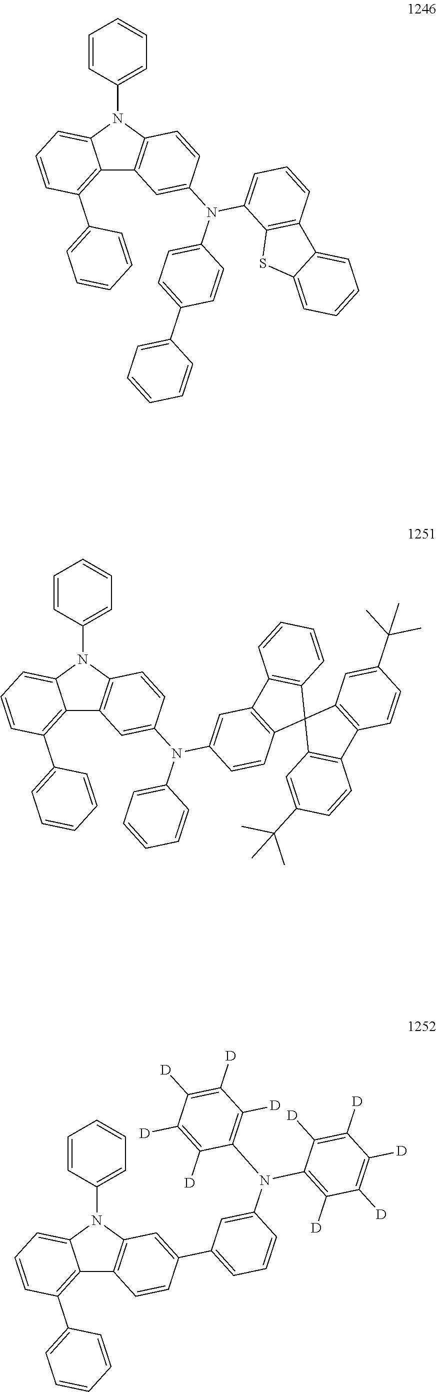 [SMILES: c1ccc(-c2ccc(N(c3ccc4c(c3)c3c(-c5ccccc5)cccc3n4-c3ccccc3)c3cccc4c3sc3ccccc34)cc2)cc1 ; CC(C)(C)c1ccc2c(c1)C1(c3ccccc3-c3cc(N(c4ccccc4)c4ccc5c(c4)c4c(-c6ccccc6)cccc4n5-c4ccccc4)ccc31)c1cc(C(C)(C)C)ccc1-2 ; [2H]c1c([2H])c([2H])c(N(c2cccc(-c3ccc4c5c(-c6ccccc6)cccc5n(-c5ccccc5)c4c3)c2)c2c([2H])c([2H])c([2H])c([2H])c2[2H])c([2H])c1[2H]]

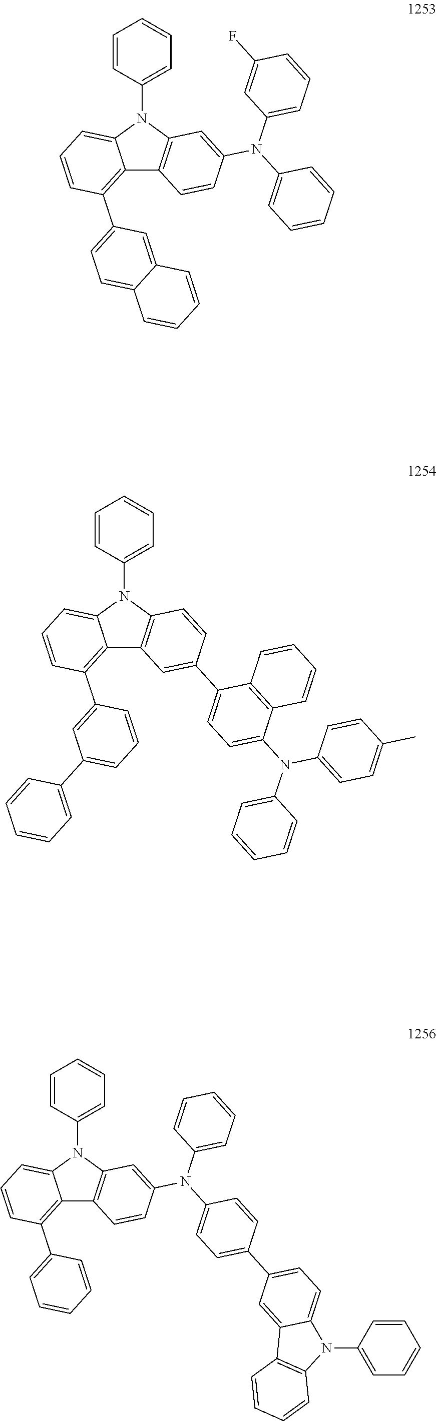 [SMILES: Fc1cccc(N(c2ccccc2)c2ccc3c4c(-c5ccc6ccccc6c5)cccc4n(-c4ccccc4)c3c2)c1 ; Cc1ccc(N(c2ccccc2)c2ccc(-c3ccc4c(c3)c3c(-c5cccc(-c6ccccc6)c5)cccc3n4-c3ccccc3)c3ccccc23)cc1 ; c1ccc(-c2cccc3c2c2ccc(N(c4ccccc4)c4ccc(-c5ccc6c(c5)c5ccccc5n6-c5ccccc5)cc4)cc2n3-c2ccccc2)cc1]

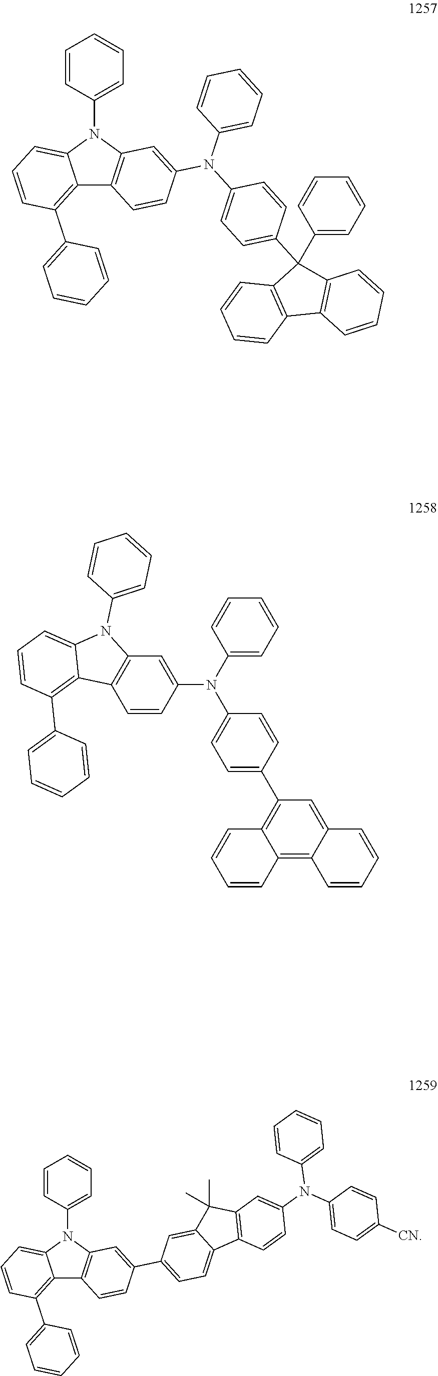 [SMILES: c1ccc(-c2cccc3c2c2ccc(N(c4ccccc4)c4ccc(C5(c6ccccc6)c6ccccc6-c6ccccc65)cc4)cc2n3-c2ccccc2)cc1 ; c1ccc(-c2cccc3c2c2ccc(N(c4ccccc4)c4ccc(-c5cc6ccccc6c6ccccc56)cc4)cc2n3-c2ccccc2)cc1 ; CC1(C)c2cc(-c3ccc4c5c(-c6ccccc6)cccc5n(-c5ccccc5)c4c3)ccc2-c2ccc(N(c3ccccc3)c3ccc(C#N)cc3)cc21]

10. The nitrogen-containing compound of claim 1, wherein the nitrogen-containing compound is selected from the following compound:

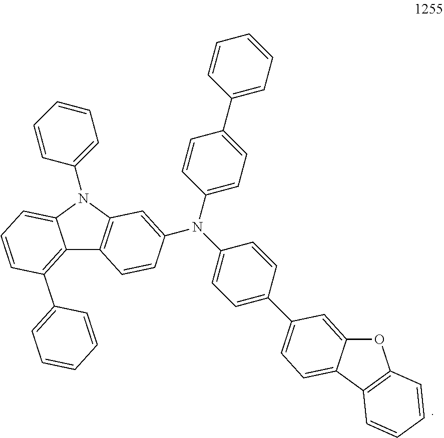 [SMILES: c1ccc(-c2ccc(N(c3ccc(-c4ccc5c(c4)oc4ccccc45)cc3)c3ccc4c5c(-c6ccccc6)cccc5n(-c5ccccc5)c4c3)cc2)cc1]

11. An electronic element, comprising an anode and a cathode which is arranged oppositely to the anode, and a functional layer disposed between the anode and the cathode; wherein the functional layer comprises the nitrogen-containing compound of claim 1.

12. The electronic element of claim 11, wherein the electronic element is an organic electroluminescent device or a photoelectric conversion device.

13. The electronic element of claim 12, wherein the electronic element is an organic electroluminescent device; and the organic electroluminescent device is a green device.

14. An electronic apparatus, comprising the electronic element of claim 11.

15. The nitrogen-containing compound of claim 3, wherein substituents in the L, L₁ and L₂ are the same or different, and are respectively and independently selected from deuterium, a halogen group, cyano, alkyl with 1 to 5 carbon atoms, aryl with 6 to 18 carbon atoms, and heteroaryl with 12 to 18 carbon atoms.

16. The nitrogen-containing compound of claim 4, wherein substituents in the L, L₁ and L₂ are the same or different, and are respectively and independently selected from deuterium, fluorine, cyano, methyl, ethyl, n-propyl, isopropyl, tert-butyl, phenyl, naphthyl, biphenyl, terphenyl, phenanthryl, dimethylfluorenyl, dibenzofuranyl, dibenzothienyl, carbazolyl, and N-phenylcarbazolyl.

17. The nitrogen-containing compound of claim 6, wherein substituents in the Ar₁ and Ar₂ are the same as or different from each other, and are respectively and independently selected from deuterium, a halogen group, cyano, alkyl with 1 to 5 carbon atoms, aryl with 6 to 20 carbon atoms, and heteroaryl with 12 to 20 carbon atoms.

18. The nitrogen-containing compound of claim 7, wherein substituent in the Ara is phenyl.

19. The electronic element of claim 11, wherein the functional layer comprises an electron blocking layer, the electron blocking layer comprises the nitrogen-containing compound.
